(19) United States
(12) Patent Application Publication
(10) Pub. No.: US 2024/0150346 A1
(43) Pub. Date: May 9, 2024

(54) CLASS OF HETEROAROMATIC COMPOUND, PREPARATION METHOD THEREFOR AND USE THEREOF

(71) Applicant: INNOVSTONE THERAPEUTICS LIMITED, Shanghai (CN)

(72) Inventors: Yunlong SONG, Shanghai (CN); Chen ZHANG, Shanghai (CN); Fang GAO, Shanghai (CN); Weimin LIU, Shanghai (CN); Qun DANG, Shanghai (CN); Pan LI, Shanghai (CN); Zhou YIN, Shanghai (CN); Xin CAI, Shanghai (CN); Xiaodan FU, Shanghai (CN); Jianbin MA, Shanghai (CN)

(21) Appl. No.: 18/263,442

(22) PCT Filed: Jan. 27, 2022
(86) PCT No.: PCT/CN2022/074172

§ 371 Date: Jul. 28, 2023

(30) Foreign Application Priority Data
Jan. 28, 2021 (CN) 202110118008.8
Dec. 1, 2021 (CN) 202111457630.8

(51) Int. Cl. C07D 471/04; C07D 471/14; C07D 487/04; C07D 519/00

(52) U.S. Cl. CPC C07D 471/04; C07D 471/14; C07D 487/04; C07D 519/00

(57) ABSTRACT

A compound, as represented by formula I, as a TLR7 agonist, a method for preparing the compound, and the use of the compound in treating diseases mediated by the TLR7 agonist are provided. Studies on the activity of a human-derived receptor, a TLR7 agonist, show that compounds have a strong agonistic effect on the human-derived receptor, TLR7, and can be used as a foreground compound for treating diseases mediated by the TLR7 agonist.

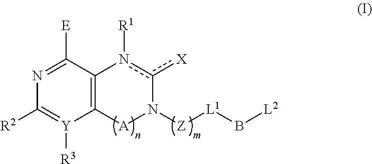

### REFERENCE TO RELATED APPLICATIONS

The present disclosure claims priority to the patent application for Invention with the application No. 202110118008.8 filled with China National Intellectual Property Administration on Jan. 28, 2021 and entitled “CLASS OF HETEROAROMATIC COMPOUND, PREPARATION METHOD THEREFOR AND USE THEREOF” and the patent application for Invention with the application No. 202111457630.8 filled with China National Intellectual Property Administration on Dec. 1, 2021 and entitled “CLASS OF HETEROAROMATIC COMPOUND, PREPARATION METHOD THEREFOR AND USE THEREOF”, which are all incorporated herein by reference in their entirety.

### TECHNICAL FIELD

The present disclosure relates to the technical field of pharmaceuticals, and particularly relates to a heteroaromatic compound, and a preparation method therefor and use thereof.

### BACKGROUND

TLRs are single transmembrane non-catalytic proteins that can identify molecules of conserved structure derived from microorganisms. TLRs can identify microorganisms and activate the body to produce an immune cell response when they break through the physical barriers of the body, such as skin and mucosa. There are 11 members of the family of human TLRs that have been identified in mammals and humans. TLR receptors for human can be divided into 5 subfamilies, i.e., TLR2, TLR3, TLR4, TLRS, and TLR9, according to chromosomal locations, genetic structures, and amino acid sequences. TLR2 subfamily includes TLR1, TLR2, TLR6, and TLR10; TLR9 subfamily includes TLR7, TLR8, and TLR9; and TLR3, TLR4, and TLR5 each form a subfamily.

TLRs play multiple roles in acquired immunity. First, TLRs play a recognition role in acquired immunity. Dendritic cells (DCs), the most potent antigen-presenting cells of the body, can express TLRs. By means of TLR recognition of PAMP-containing molecules such as LPS, GpG-DNA, peptidoglycan, lipoprotein and cell wall components of Mycobacterium, dendritic cells are activated and matured to provide co-stimulatory signals for acquired immunity. TLRs are therefore bridges for microbial components to cause the activation of dendritic cells. Second, TLRs play a regulatory role in the type of acquired immunity response. Most TLRs, when activated, can induce the antimicrobial defense system to produce IL-1β, IL-6, and TNF, as well as chemotactic cytokines, thereby regulating the balance of Th1 and Th2 in the body.

TLR7 is one of the members of the TLR family. It was previously thought that its primary role was only to identify viral single-stranded RNA (mRNA) to mediate the innate immune response against viruses. With the intensive research on TLR7, TLR7 was found to have different functions not only in the innate immune response against viruses, but also in the fields of immunodeficiency diseases, tumor resistance, immunoregulation, and the like.

TLR7 has its unique advantages over other immunomodulatory targets: 1) the activation of TLR7 is an effective mechanism for activating pDCs, which are the key immune switch linking the innate immunity and the adaptive immunity; 2) the TLR7 agonist can be safely and effectively combined with other immunotherapy drugs; 3) the expression of TLR7 is limited to professional immune cells (pDC, B); and this may be associated with relatively low toxic and side effects; 4) the oral small molecule agonist for TLR7 can reach the target, while other targets are limited to intratumoral injection or iv administration at present; 5) the mechanism of action of TLR7 has been clinically validated; and 6) because the TLR7 small molecule binding site has relatively unique structural features, the probability of off-target binding is very low.

ANA773 is an oral TLR7 agonist from Anadys. It is a prodrug, and the effective molecule is ANA122. ANA773 is converted in vivo to the effective ingredient ANA122 by hydrolysis and oxidation. In clinical phase I, ANA773 demonstrated safety in cancer patients. Enhanced immune function was demonstrated in healthy volunteers. Clinical trials were conducted in both cancer and HCV patients.

Primmune is also developing an oral agonist for TLR7, which is currently in the preclinical stage. Their primary molecule, PRX034, demonstrated good PK and PD in animal experiments.

The patent WO2019126242A1 of BMS also discloses an aminoindole compound as a TLR inhibitor, but no clinical report is available.

To sum up, the TLR7 activator can be used as a potentially powerful new anti-cancer drug. Researches on development of new drugs targeting TLR7 have a positive gap-filling effect on solving unmet clinical requirements.

### SUMMARY

The object of the present disclosure is to provide a compound with a brand-new structure as a TLR7 activator, a preparation method for the compound, and use of the compound in treating diseases mediated by a TLR7 activator.

The present disclosure provides a compound represented by the following formula (I), and a stereoisomer, an optical isomer, a pharmaceutically acceptable salt, a prodrug, and a solvate thereof,

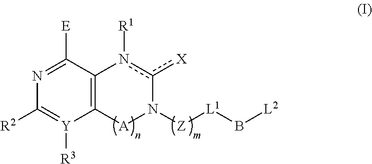

wherein,
E is selected from hydrogen, amino, and halogen;
R¹ is selected from hydrogen, hydroxyl, amino, C₁₋₆ alkyl, C₃₋₆ cycloalkyl, 4- to 6-membered heterocyclyl, aryl, and 5- to 6-membered heteroaryl, or R¹ is absent;
the compound fragment

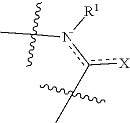

is selected from

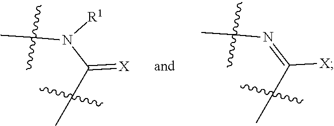

when the compound fragment is selected from

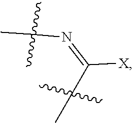

R¹ is absent;
when

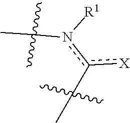

is selected from

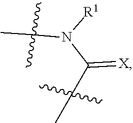

X is absent, or X is selected from O, S, C(R⁸)(R⁹), and N(R⁸); wherein, R⁸ or R⁹, at each occurrence, is independently selected from hydrogen, halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl;
when

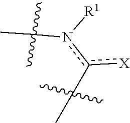

is selected from

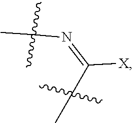

X is selected from hydrogen, acylamino, formyl, acetyl, carboxyl, cyano, —O(R²⁰), —S(O)_{f}(R²⁰), optionally substituted C₁₋₆ alkyl, optionally substituted —OC₁₋₆ alkyl, optionally substituted —SC₁₋₆ alkyl, optionally substituted —OC₃₋₆ cycloalkyl, optionally substituted —SC₃₋₆ cycloalkyl, optionally substituted —COOC₁₋₆ alkyl, optionally substituted —SC₁₋₆ alkyl-COOC₁₋₆ alkyl, optionally substituted —OC₁₋₆ alkyl-COOC₁₋₆ alkyl, and optionally substituted —SC₁₋₆ alkyl-OC₁₋₆ alkyl, the “optionally substituted” means being unsubstituted or being substituted with one or more substituents selected from hydroxyl, carboxyl, halogen, cyano, amino, acylamino, C₁₋₆ alkyl, C₂₋₆ alkenyl, C₃₋₆ cycloalkyl, methoxy, and methylthio; wherein, R²⁰, at each occurrence, is independently selected from hydrogen, halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl; f is selected from 0, 1, and 2;
R² is selected from hydrogen, halogen, hydroxyl, sulfhydryl, amino, cyano, R⁴, —O(R⁴), —S(R⁴), —N(R⁴)(R⁵),

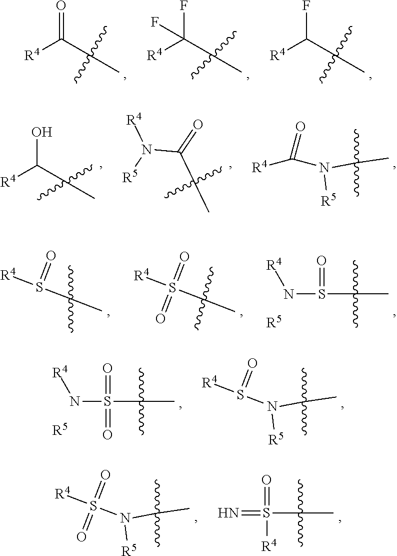

—PO(R⁴), —N(R⁵)PO(R⁴), and —PON(R⁵)(R⁴); R⁴ or R⁵, at each occurrence, is independently selected from C₁₋₆ alkyl, C₁₋₆ haloalkyl, C₂₋₆ alkenyl, C₂₋₆ alkynyl, C₃₋₆ cycloalkyl, 4- to 6-membered heterocyclyl, aryl, and 5- to 6-membered heteroaryl; A, at each occurrence, is independently selected from —CO— and —C(R⁶)(R⁷)—; wherein, R⁶ or R⁷, at each occurrence, is independently selected from hydrogen, halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl; n is selected from 0, 1, and 2;
Y is selected from C and N;
when Y is selected from N, R³ is absent;
when Y is selected from C, R³ is selected from hydrogen, halogen, hydroxyl, sulfhydryl, amino, cyano, C₁₋₆ alkyl, C₃₋₆ cycloalkyl, 4- to 6-membered heterocyclyl, aryl, and 5- to 6-membered heteroaryl; alternatively, R² and R³, together with the atom connected thereto, form an H ring, the H ring being an optionally substituted 4- to 10-membered ring selected from C₄₋₈ cycloalkene, 4- to 6-membered heterocyclyl ring, phenyl ring, and 5- to 6-membered heteroaryl ring; the “optionally substituted” refers to being unsubstituted or being substituted with one or more groups selected from halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, 5- to 6-membered heteroaryl, —NH(C₁₋₆ alkyl), —N(C₁₋₆ alkyl)(C₁₋₆ alkyl), —OC₁₋₆ alkyl, —SC₁₋₆ alkyl, —SOC₁₋₆ alkyl, and —SO(NH)(C₁₋₆ alkyl); Z, at each occurrence, is independently selected from —O—, —S—, —C(R¹⁰)(R¹¹)—, —CO—, —CS—, —CO₂—, —CON(R¹⁰)—, —SON(R¹⁰)—, —SO₂N(R¹⁰)—, —N(R¹⁰)—, —SO—, —SO₂—, and —P(O)(R¹⁰)—; wherein, R¹⁰ or R¹¹, at each occurrence, is independently selected from hydrogen, halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl; m is selected from 0, 1, 2, and 3;
B is selected from -(chemical bond), —O—, —S—, —N(R¹²)—, —CO—, —SO—, —SO₂—, —(CH₂)ₚN(R¹²)—, —N(R¹²)(CH₂)ₚ—, —S(O)N(R¹²)—, —S(O)₂N(R¹²)—, —N(R¹²)SO—, —N(R¹²)S(O)₂—, —C(O)N(R¹²)—, —N(R¹²)C(O)—, —C(R¹²)(R¹³)—, and —C(R¹²)(R¹³)—C(R¹²)(R¹³)—; wherein, p=0, 1, 2, or 3, and R¹² or R¹³, at each occurrence, is independently selected from hydrogen, deuterium, halogen, hydroxyl, sulfhydryl, amino, cyano, optionally substituted C₁₋₆ alkyl, optionally substituted C₂₋₆ alkenyl, optionally substituted C₂₋₆ alkynyl, optionally substituted C₃₋₆ cycloalkyl, optionally substituted 4- to 6-membered heterocyclyl, optionally substituted phenyl, and optionally substituted 5- to 6-membered heteroaryl; the “optionally substituted” refers to being unsubstituted or being substituted with one or more groups selected from halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl;
L¹ is selected from optionally substituted C₁₋₁₂ alkyl, optionally substituted C₂₋₁₂ alkenyl, optionally substituted C₂₋₁₂ alkynyl, optionally substituted C₆₋₁₀ aryl, optionally substituted 5- to 10-membered heteroaryl, optionally substituted C₃₋₈ cycloalkyl, and optionally substituted 4- to 10-membered heterocyclyl; the “optionally substituted” refers to being unsubstituted or being substituted with one or more R¹⁴; wherein, R¹⁴, at each occurrence, is independently selected from hydrogen, halogen, hydroxyl, sulfhydryl, amino, nitro, cyano, —R¹⁵, —OR¹⁵, —SR¹⁵, SO(R¹⁵), —SO₂(R¹⁵), —COR¹⁵, —COOR¹⁵, —N(R¹⁵)(R¹⁶), —CONHR¹⁵, —CON(R¹⁵)(R¹⁶), —SONH(R¹⁵), —SON(R¹⁵)(R¹⁶), SO₂NH(R¹⁵), and —SO₂N(R¹⁵)(R¹⁶); wherein, R¹⁵ or R¹⁶, at each occurrence, is independently selected from C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl, which may be optionally substituted with one or more of hydrogen, halogen, hydroxyl, sulfhydryl, amino, cyano, C₁₋₆ alkyl, C₃₋₆ cycloalkyl, 4- to 6-membered heterocyclyl, aryl, and 5- to 6-membered heteroaryl;
L² is absent, or L² is selected from optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted 4- to 10-membered heterocyclyl, optionally substituted C₆₋₁₀ aryl, optionally substituted 5- to 10-membered heteroaryl, optionally substituted —NHC₁₋₆ alkyl, and optionally substituted —N(C₁₋₆ alkyl)₂; the “optionally substituted” refers to being unsubstituted or being substituted with one or more R¹⁷, wherein, R¹⁷, at each occurrence, is independently selected from hydrogen, halogen, hydroxyl, sulfhydryl, nitro, amino, cyano, oxo, —R¹⁸, —OR¹⁸, —SR¹⁸, —SO(R¹⁸), —SO₂(R¹⁸), —COOR¹⁸, —COR¹⁸, —NH(R¹⁸), —N(R¹⁸)(R¹⁹), —CONHR¹⁹, —CON(R¹⁸)(R¹⁹), —SONH(R¹⁸), —SON(R¹⁸)(R¹⁹), SO₂NH(R¹⁸), —SO₂N(R¹⁸)(R¹⁹), —C₀₋₃ alkyl-N(R¹⁸)(R¹⁹), —COC₁₋₃ alkyl-O(R¹⁸), —COC₁₋₃ alkyl-NH₂, and —C₁₋₃ alkyl-N(R¹⁸)(R¹⁹); wherein, the oxo means that two H at the same substitution site are substituted with the same O to form a divalent substituent ═O, and R¹⁸ or R¹⁹, at each occurrence, is independently selected from hydrogen, C₁₋₆ alkyl, C₁₋₆ haloalkyl, C₁₋₆ alkenyl, C₁₋₆ alkynyl, C₃₋₆ cycloalkyl, 4- to 6-membered heterocyclyl, aryl, and 5- to 6-membered heteroaryl.

Unless otherwise stated, the heteroatoms in the heteroaryl and heterocyclyl described above are independently selected from O, N, and S, and the number of the heteroatoms is 1, 2, 3, or 4.

In one embodiment of the present disclosure, the compound represented by formula (I) is further represented by formula (II):

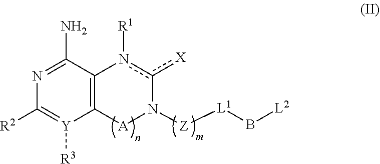

wherein,

R¹ is selected from hydrogen, hydroxyl, amino, C₁₋₆ alkyl, C₃₋₆ cycloalkyl, 4- to 6-membered heterocyclyl, aryl, and 5- to 6-membered heteroaryl;
the compound fragment

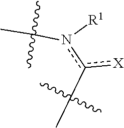

is selected from

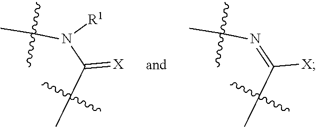

when the compound fragment is selected from

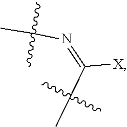

R¹ is absent;
when

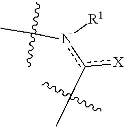

is selected from

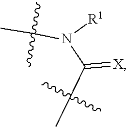

X is absent, or X is selected from O, S, C(R⁸)(R⁹), and N(R⁸); wherein, R⁸ or R⁹, at each occurrence, is independently selected from hydrogen, halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl;
when

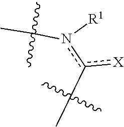

is selected from

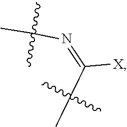

X is selected from hydrogen, cyano, —O(R²⁰), and —S(O)_{f}(R²⁰); wherein, R²⁰, at each occurrence, is independently selected from hydrogen, halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl; f is selected from 0, 1, and 2; R² is selected from hydrogen, halogen, hydroxyl, sulfhydryl, amino, cyano, R⁴, —O(R⁴), —S(R⁴), —N(R⁴)(R⁵),

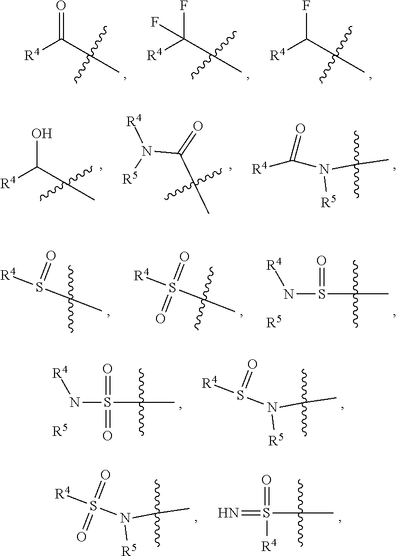

—PO(R⁴), —N(R⁵)PO(R⁴), and —PON(R⁵)(R⁴); R⁴ or R⁵, at each occurrence, is independently selected from C₁₋₆ alkyl, C₁₋₆ haloalkyl, C₂₋₆ alkenyl, C₂₋₆ alkynyl, C₃₋₆ cycloalkyl, 4- to 6-membered heterocyclyl, aryl, and 5- to 6-membered heteroaryl; A, at each occurrence, is independently selected from —CO— and —C(R⁶)(R⁷)—; wherein, R⁶ or R⁷, at each occurrence, is independently selected from hydrogen, halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl; n is selected from 0, 1, and 2;
Y is selected from C and N;
when Y is selected from N, R³ is absent;
when Y is selected from C, R³ is selected from hydrogen, halogen, hydroxyl, sulfhydryl, amino, cyano, C₁₋₆ alkyl, C₃₋₆ cycloalkyl, 4- to 6-membered heterocyclyl, aryl, and 5- to 6-membered heteroaryl; alternatively, R² and R³ may be connected to form an H ring, the H ring being an optionally substituted 4- to 10-membered ring selected from C₄₋₈ cycloalkene, 4- to 6-membered heterocyclyl ring, phenyl ring, and 5- to 6-membered heteroaryl ring; the “optionally substituted” refers to being unsubstituted or being substituted with one or more groups independently selected from halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl;
Z, at each occurrence, is independently selected from —O—, —S—, —C(R¹⁰)(R¹¹)—, —CO—, —CS—, —CO₂—, —CON(R¹⁰)—, —SON(R¹⁰)—, —SO₂N(R¹⁰)—, —N(R¹⁰)—, —SO—, —SO₂—, and —P(O)(R¹⁰)—; wherein, R¹⁰ or R¹¹, at each occurrence, is independently selected from hydrogen, halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl; m is selected from 0, 1, 2, and 3;
B is selected from -(chemical bond), —O—, —S—, —N(R¹²)—, —CO—, —SO—, —SO₂—, —(CH₂)ₚN(R¹²)—, —N(R¹²)(CH₂)ₚ—, —S(O)N(R¹²)—, —S(O)₂N(R¹²)—, —N(R¹²)SO—, —N(R¹²)S(O)₂—, —C(O)N(R¹²)—, —N(R¹²)C(O)—, and —C(R¹²)(R¹³)—; wherein, p=0, 1, 2, or 3, and R¹² or R¹³, at each occurrence, is independently selected from hydrogen, halogen, hydroxyl, sulfhydryl, amino, cyano, optionally substituted C₁₋₆ alkyl, optionally substituted C₂₋₆ alkenyl, optionally substituted C₂₋₆ alkynyl, optionally substituted C₃₋₆ cycloalkyl, optionally substituted 4- to 6-membered heterocyclyl, optionally substituted C₆ aryl, and optionally substituted 5- to 6-membered heteroaryl; the “optionally substituted” refers to being unsubstituted or being substituted with one or more groups independently selected from halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl;
L¹ is selected from optionally substituted C₁₋₁₂ alkyl, optionally substituted C₂₋₁₂ alkenyl, optionally substituted C₂₋₁₂ alkynyl, optionally substituted C₆₋₁₀ aryl, optionally substituted 5- to 10-membered heteroaryl, optionally substituted C₃₋₇ cycloalkyl, and optionally substituted 4- to 10-membered heterocyclyl; the “optionally substituted” refers to being unsubstituted or being substituted with one or more R¹⁴; wherein, R¹⁴, at each occurrence, is independently selected from hydrogen, halogen, hydroxyl, sulfhydryl, amino, nitro, cyano, —R¹⁵, —OR¹⁵, —SR¹⁵, SO(R¹⁵), —SO₂(R¹⁵), —COR¹⁵, —COOR¹⁵, —N(R¹⁵)(R¹⁶), —CONHR¹⁵, —CON(R¹⁵)(R¹⁶), —SONH(R¹⁵), —SON(R¹⁵)(R¹⁶), SO₂NH(R¹⁵), and —SO₂N(R¹⁵)(R¹⁶); wherein, R¹⁵ or R¹⁶, at each occurrence, is independently selected from C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl, which may be optionally substituted with one or more of hydrogen, halogen, hydroxyl, sulfhydryl, amino, cyano, C₁₋₆ alkyl, C₃₋₆ cycloalkyl, 4- to 6-membered heterocyclyl, aryl, and 5- to 6-membered heteroaryl;
L² is absent, or L² is selected from optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted 4- to 10-membered heterocyclyl, optionally substituted C₆₋₁₀ aryl, and optionally substituted 5- to 10-membered heteroaryl; the “optionally substituted” refers to being unsubstituted or being substituted with one or more R¹⁷, wherein, R¹⁷, at each occurrence, is independently selected from hydrogen, halogen, hydroxyl, sulfhydryl, nitro, amino, cyano, oxo, —R¹⁸, —OR¹⁸, —SR¹⁸, —SO(R¹⁸), —SO₂(R¹⁸), —COOR¹⁸, —COR¹⁸, —NH(R¹⁸), —N(R¹⁸)(R¹⁹), —CONHR¹⁹, —CON(R¹⁸)(R¹⁹), —SONH(R¹⁸), —SON(R¹⁸)(R¹⁹), SO₂NH(R¹⁸), —SO₂N(R¹⁸)(R¹⁹), and —C₀₋₃ alkyl-N(R¹⁸)(R¹⁹); wherein, the oxo means that two H at the same substitution site are substituted with the same O to form a divalent sub stituent ═O, and R¹⁸ or R¹⁹, at each occurrence, is independently selected from hydrogen, C₁₋₆ alkyl, C₁₋₆ haloalkyl, C₂₋₆ alkenyl, C₂₋₆ alkynyl, C₃₋₆ cycloalkyl, 4- to 6-membered heterocyclyl, aryl, and 5- to 6-membered heteroaryl.

In one embodiment of the present disclosure, the compound represented by formula (II) is further represented by formula (II)a:

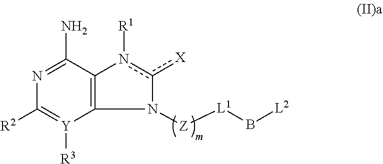

wherein, each of the substituents in formula (II)a is as defined in formula (II).

In one embodiment of the present disclosure, the compound represented by formula (II) is further represented by formula (III)a:

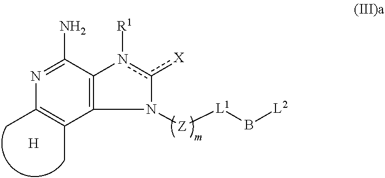

wherein, the H ring in the formula (III)a is an optionally substituted 4- to 10-membered ring selected from C₄₋₈ cycloalkene, 4- to 6-membered heterocyclyl ring, phenyl ring, and 5- to 6-membered heteroaryl ring; the “optionally substituted” refers to being unsubstituted or being substituted with one or more groups independently selected from halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl, and each of the other substituents is as defined in formula (II).

In one embodiment of the present disclosure, the compound represented by formula (II) is further represented by formula (II)b:

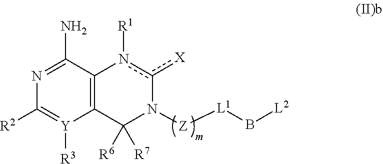

wherein, each of the substituents in formula (II)b is as defined in formula (II).

In a preferred embodiment, R¹ is selected from hydrogen, C₁₋₆ alkyl, C₃₋₆ cycloalkyl, 4- to 6-membered heterocyclyl, aryl, and 5- to 6-membered heteroaryl.

In a more preferred embodiment, R¹ is selected from hydrogen, methyl, ethyl, n-propyl, isopropyl, n-butyl, sec-butyl, isobutyl, tert-butyl, cyclopropyl, cyclobutyl, cyclopentyl, and cyclohexyl.

In a further preferred embodiment, R¹ is selected from hydrogen, methyl, and cyclopropyl.

In a preferred embodiment, R² is selected from hydrogen, halogen, hydroxyl, sulfhydryl, amino, cyano, R⁴, —O(R⁴), —S(R⁴), —N(R⁴)(R⁵),

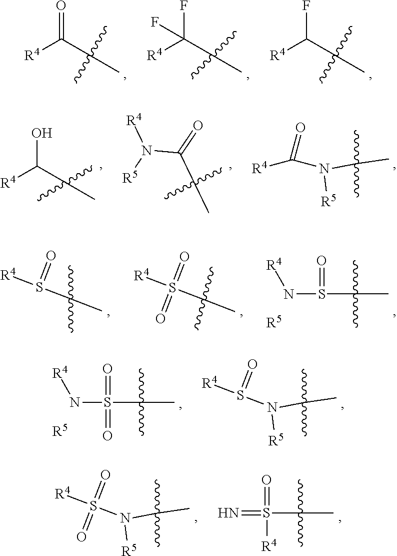

—PO(R⁴), —N(R⁵)PO(R⁴), and —PON(R⁵)(R⁴), and R⁴ or R⁵, at each occurrence, is independently selected from C₁₋₆ alkyl, C₁₋₃ haloalkyl, C₂₋₆ alkenyl, and C₂₋₆ alkynyl.

In a more preferred embodiment, R² is selected from hydrogen, halogen, hydroxyl, sulfhydryl, amino, cyano, trifluoromethyl, —CH₂CF₃, trifluoromethoxy, —OCH₂CF₃, —R⁴, —O(R⁴), —S(R⁴), —N(R⁴)(R⁵),

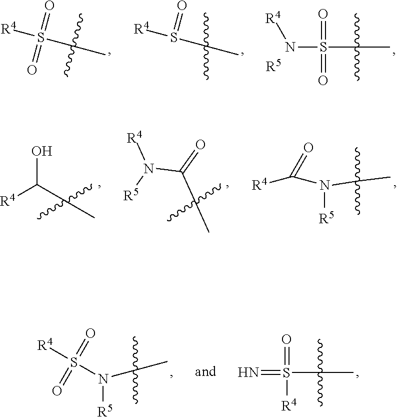

and R⁴ or R⁵, at each occurrence, is independently selected from C₁₋₆ alkyl, C₂₋₆ alkenyl, and C₂₋₆ alkynyl.

In a further preferred embodiment, R² is selected from hydrogen, halogen, hydroxyl, sulfhydryl, amino, cyano, trifluoromethyl, trifluoromethoxy, —C₁₋₆ alkyl, —C₂₋₆ alkenyl, —C₂₋₆ alkynyl, —O—C₁₋₆ alkyl, —S—C₁₋₆ alkyl, —NH—C₁₋₆ alkyl,

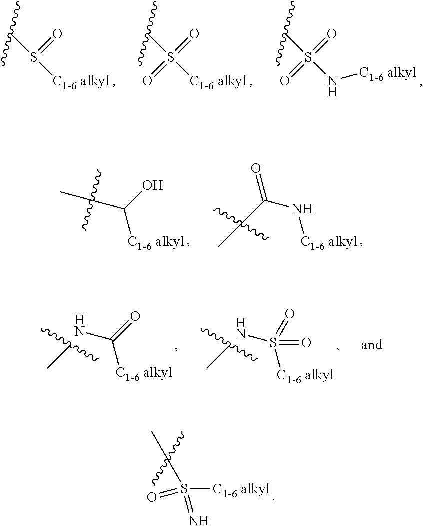

In a preferred embodiment, A, at each occurrence, is independently selected from —CO— and —C(R⁶)(R⁷)—; wherein, R⁶ or R⁷, at each occurrence, is independently selected from hydrogen, halogen, hydroxyl, sulfhydryl, amino, cyano, methyl, ethyl, propyl, tert-butyl, cyclopropyl, ethenyl, and ethynyl; n is selected from 0, 1, and 2.

In a preferred embodiment

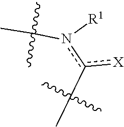

is selected from

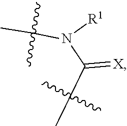

and X is selected from O, S, C(R⁸)(R⁹), and N(R⁸); wherein, R⁸ or R⁹, at each occurrence, is independently selected from hydrogen, halogen, hydroxyl, sulfhydryl, amino, cyano, C₁₋₃ alkyl, C₂₋₃ alkenyl, C₂₋₃ alkynyl, and C₃₋₆ cycloalkyl, and preferably, R⁸ or R⁹, at each occurrence, is independently selected from hydrogen, halogen, hydroxyl, methyl, and cyclopropyl.

In a more preferred embodiment,

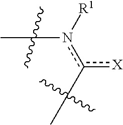

is selected from

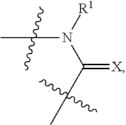

and X is selected from O, S, CH₂, CHF, CHOH, CF₂, NH, and NCH₃, preferably O, S, and CH₂, more preferably O.

In another preferred embodiment,

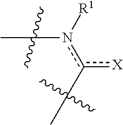

is selected from

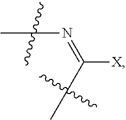

and X is selected from —O(R²⁰) and —S(O)_{f}(R²⁰); wherein, R²⁰, at each occurrence, is independently selected from hydrogen, halogen, hydroxyl, sulfhydryl, amino, cyano, C₁₋₄ alkyl, C₂₋₃ alkenyl, C₂₋₃ alkynyl, C₃₋₆ cycloalkyl, 4- to 6-membered heterocyclyl, aryl, and 5- to 6-membered heteroaryl, and preferably, R²⁰, at each occurrence, is independently selected from hydrogen, halogen, hydroxyl, methyl, ethyl, n-propyl, n-butyl, isopropyl, cyclopropyl, cyclobutyl, and cyclopentyl; f is selected from 0 and 1.

In another more preferred embodiment,

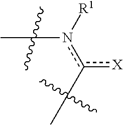

is selected from

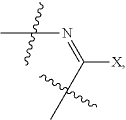

and X is selected from SCH₃, SCH₂CH₃,

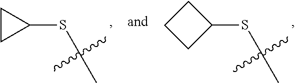 [SMILES: *SC1CC1 ; *SC1CCC1]

preferably SCH₃ and

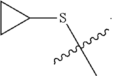 [SMILES: *SC1CC1]

In a preferred embodiment, Y is selected from N, and R³ is absent.

In another preferred embodiment, Y is selected from C, and R³ is selected from hydrogen, halogen, hydroxyl, sulfhydryl, amino, cyano, C₁₋₆ alkyl, C₃₋₆ cycloalkyl, 4- to 6-membered heterocyclyl, aryl, and 5- to 6-membered heteroaryl, preferably hydrogen, halogen, hydroxyl, sulfhydryl, amino, cyano, methyl, ethyl, n-propyl, isopropyl, cyclopropyl, C₅₋₆ cycloalkyl, 5- to 6-membered heterocycloalkyl, C₆ aryl, and 5- to 6-membered heteroaryl.

In yet another preferred embodiment, when Y is selected from C, R² and R³ may be connected to form an H ring, the H ring being an optionally substituted 4- to 10-membered ring selected from C₄₋₈ cycloalkene, 4- to 6-membered heterocyclyl ring, phenyl ring, and 5- to 6-membered heteroaryl ring; the “optionally substituted” refers to being unsubstituted or being substituted with one or more groups independently selected from halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl; preferably, the “optionally substituted” refers to being unsubstituted or being substituted with one or more groups independently selected from halogen, hydroxyl, sulfhydryl, amino, cyano, methyl, ethyl, n-propyl, isopropyl, cyclopropyl, n-butyl, isobutyl, cyclobutyl, ethenyl, ethynyl, 5- to 6-membered heterocyclyl, C₆ aryl, and 5- to 6-membered heteroaryl.

In a preferred embodiment, Z, at each occurrence, is independently selected from —O—, —S—, —C(R¹⁰)(R¹¹)—, —CO—, —CS—, —CO₂—, —CON(R¹⁰)—, —SON(R¹⁰)—, —SO₂N(R¹⁰)—, —N(R¹⁰)—, —SO—, and —SO₂—, preferably —C(R¹⁰)(R¹¹)—, —CO—, —CON(R¹⁰)—, —N(R¹⁰)—, and —SO₂—, more preferably —C(R¹⁰)(R¹¹)—, —CO—, —N(R¹⁰)—, and —SO₂—; wherein, R¹⁰ or R¹¹, at each occurrence, is independently selected from hydrogen, halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl, preferably hydrogen, halogen, hydroxyl, sulfhydryl, amino, cyano, C₁₋₄ alkyl, C₁₋₄ alkenyl, C₁₋₄ alkynyl, C₃₋₆ cycloalkyl, 4- to 6-membered heterocyclyl, C₆ aryl, and 5- to 6-membered heteroaryl, more preferably hydrogen, halogen, hydroxyl, sulfhydryl, amino, cyano, methyl, ethyl, n-propyl, isopropyl, and cyclopropyl.

In a preferred embodiment, m is 1 or 2.

In a preferred embodiment, B is selected from -(chemical bond), —O—, —S—, —N(R¹²)—, —CO—, —SO—, —SO₂—, —(CH₂)ₚN(R¹²)—, —N(R¹²)(CH₂)ₚ—, —S(O)N(R¹²)—, —S(O)₂N(R¹²)—, —N(R¹²)SO—, —N(R¹²)S(O)₂—, —C(O)N(R¹²)—, —N(R¹²)C(O)—, and —C(R¹²)(R¹³)—; wherein, p=0, 1, 2, or 3; R¹² or R¹³, at each occurrence, is independently selected from hydrogen, halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, C₆ aryl, and 5- to 6-membered heteroaryl;
preferably, B is selected from -(chemical bond), —O—, —S—, —N(R¹²)—, —CO—, —SO—, —SO₂—, —(CH₂)ₚN(R¹²)—, —N(R¹²)(CH₂)ₚ—, —S(O)N(R¹²)—, —S(O)₂N(R¹²)—, —N(R¹²)SO—, —N(R¹²)S(O)₂—, —C(O)N(R¹²)—, —N(R¹²)C(O)—, and —C(R¹²)(R¹³)—, wherein, p=0, 1, 2, or 3; R¹² or R¹³, at each occurrence, is independently selected from hydrogen, halogen, hydroxyl, C₁₋₆ alkyl, C₃₋₆ cycloalkyl, and 4- to 6-membered heterocyclyl.

In a more preferred embodiment, B is selected from -(chemical bond), —O—, —S—, —NH—, —CO—, —S(O)₂NH—, —NHS(O)₂—, —C(O)NH—, —NHC(O)—, —CH₂—, —CHF—, —CF₂—, —CH(OH)—, —CH(CH₃)—, —CH₂N(CH₃)—, —CH₂NH—, —N(CH₃)CH₂—, and —NHCH₂—, preferably -(chemical bond), —O—, —NH—, —CO—, —CH₂—, —CH(CH₃)—, —CH₂N(CH₃)—, and —CH₂NH—.

In a preferred embodiment, L¹ is selected from optionally substituted C₃₋₆ alkyl, optionally substituted C₆₋₁₀ aryl, and optionally substituted 5- to 10-membered heteroaryl; the “optionally substituted” refers to being unsubstituted or being substituted with one or more R¹⁴; wherein, R¹⁴ at each occurrence, is independently selected from hydrogen, halogen, hydroxyl, nitro, cyano, —R¹⁵, —OR¹⁵, —SR¹⁵, —SO₂(R¹⁵), —COR¹⁵, —COOR¹⁵, —N(R¹⁵)(R¹⁶), —CONHR¹⁵, —CON(R¹⁵)(R¹⁶), SO₂NH(R¹⁵), and —SO₂N(R¹⁵)(R¹⁶); wherein, R¹⁵ or R¹⁶, at each occurrence, is independently selected from C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl, which may be optionally substituted with one or more of hydrogen, halogen, hydroxyl, sulfhydryl, amino, cyano, C₁₋₆ alkyl, and C₃₋₆ cycloalkyl;
preferably, L¹ is selected from optionally substituted C₃₋₆ alkyl, optionally substituted C₆₋₁₀ aryl, and optionally substituted 5- to 10-membered heteroaryl; the “optionally substituted” refers to being unsubstituted or being substituted with one or more R¹⁴; wherein, R¹⁴, at each occurrence, is independently selected from hydrogen, halogen, hydroxyl, sulfhydryl, nitro, amino, cyano, —R¹⁵—, —OR¹⁵, —SR¹⁵, —SO₂(R¹⁵), —COR¹⁵, —COOR¹⁵, —N(R¹⁵)(R¹⁶), —CONHR¹⁵, —CON(R¹⁵)(R¹⁶), SO₂NH(R¹⁵), and —SO₂N(R¹⁵)(R¹⁶); wherein, R¹⁵ or R¹⁶, at each occurrence, is independently selected from C₁₋₆ alkyl, C₁₋₆ haloalkyl, C₂₋₆ alkenyl, C₂₋₆ alkynyl, C₃₋₆ cycloalkyl, 4- to 6-membered heterocyclyl, aryl, and 5- to 6-membered heteroaryl;
more preferably, L¹ is selected from optionally substituted C₃₋₆ alkyl, optionally substituted C₆ aryl, and optionally substituted 5- to 6-membered heteroaryl; the “optionally substituted” refers to being unsubstituted or being substituted with one or more R¹⁴; wherein, R¹⁴, at each occurrence, is independently selected from hydrogen, halogen, hydroxyl, sulfhydryl, nitro, amino, cyano, formyl, acetyl, propionyl, methoxycarbonyl, ethoxycarbonyl, C₁₋₆ alkyl, C₁₋₆ haloalkyl, C₁₋₆ alkoxy, C₁₋₆ haloalkoxy, C₁₋₆ alkylamino, C₂₋₆ alkenyl, C₂₋₆ alkynyl, C₃₋₆ cycloalkyl, 4- to 6-membered heterocyclyl, C₆ aryl, and 5- to 6-membered heteroaryl.

In a more preferred embodiment, L¹ is selected from the following groups optionally substituted with R¹⁴:

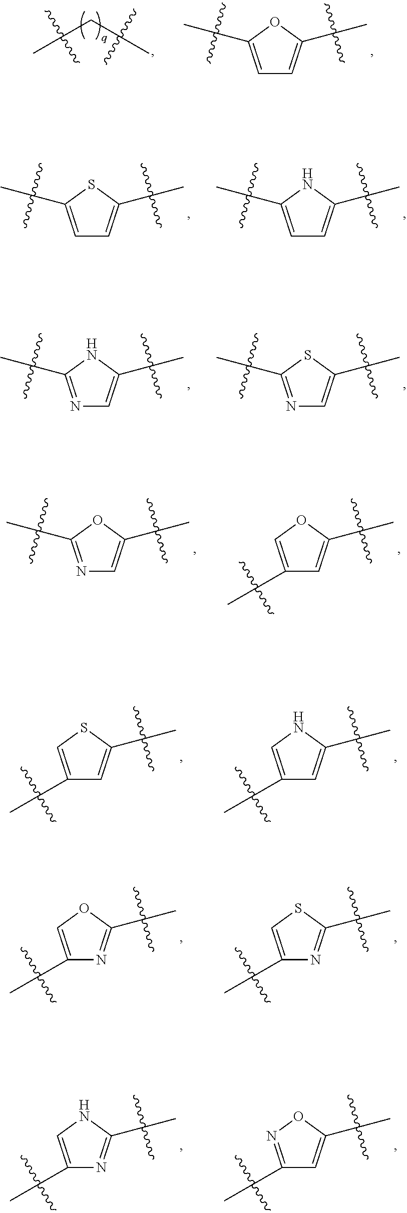

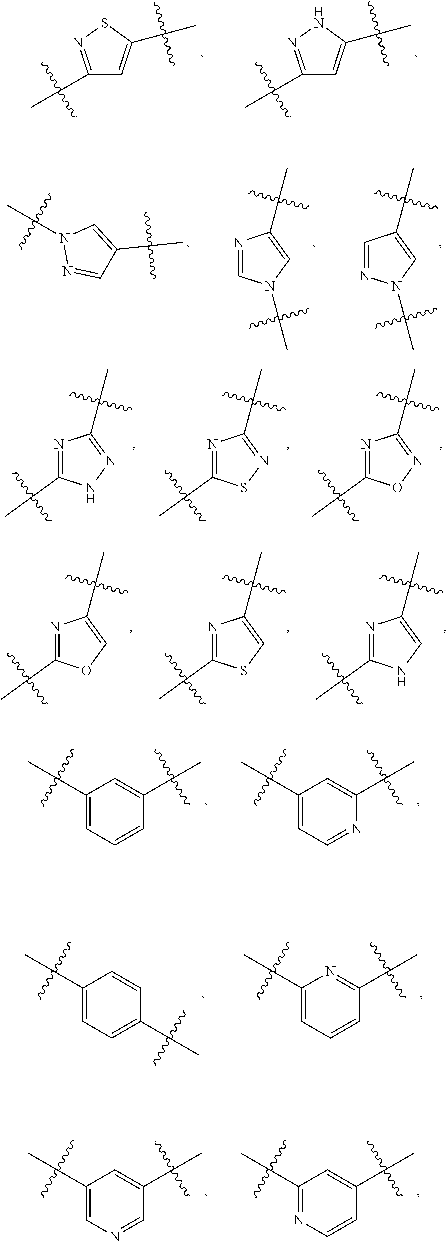

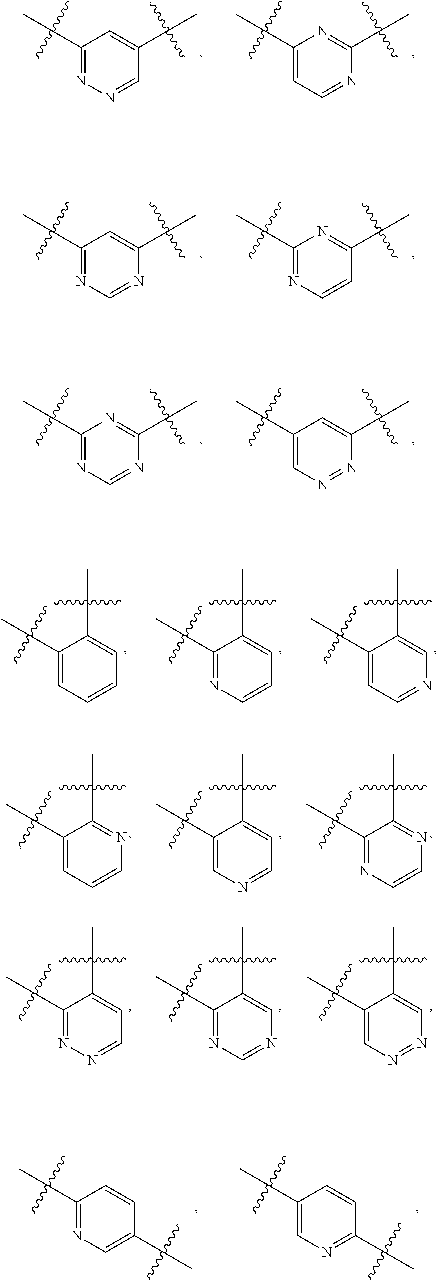

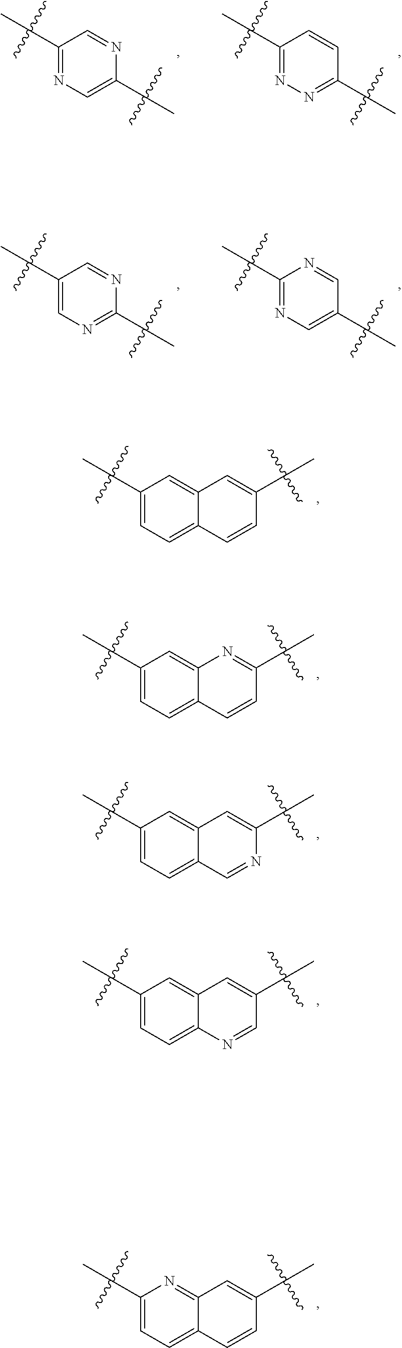

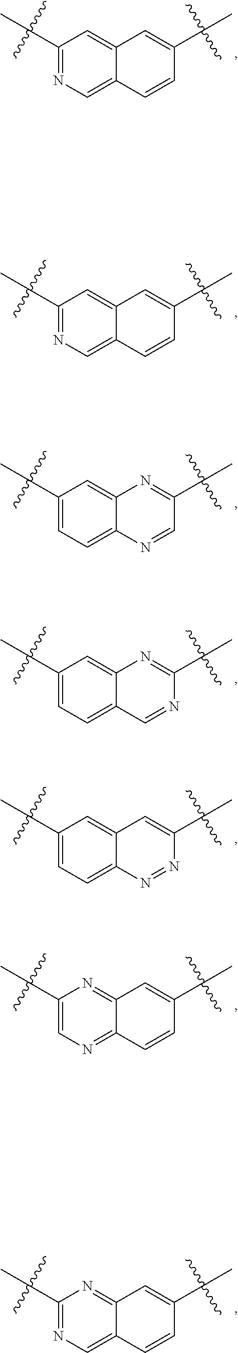

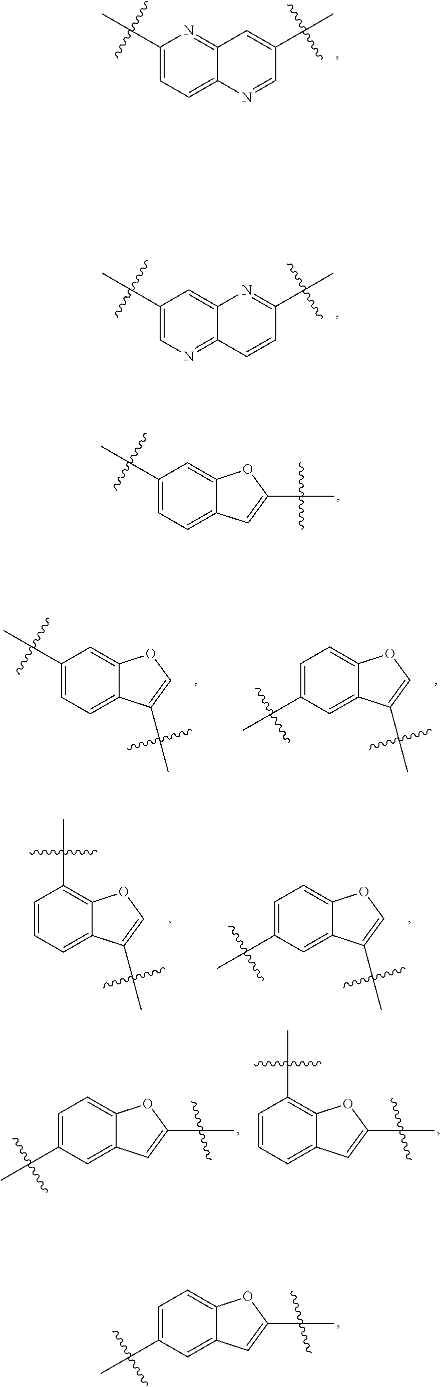

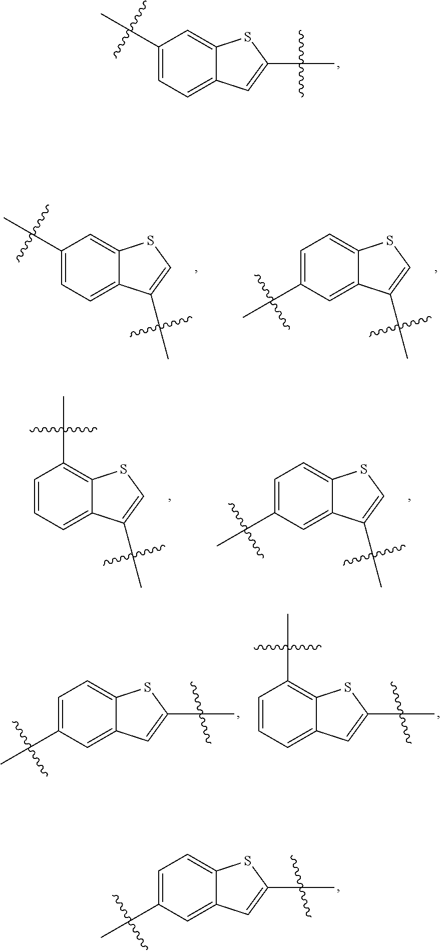

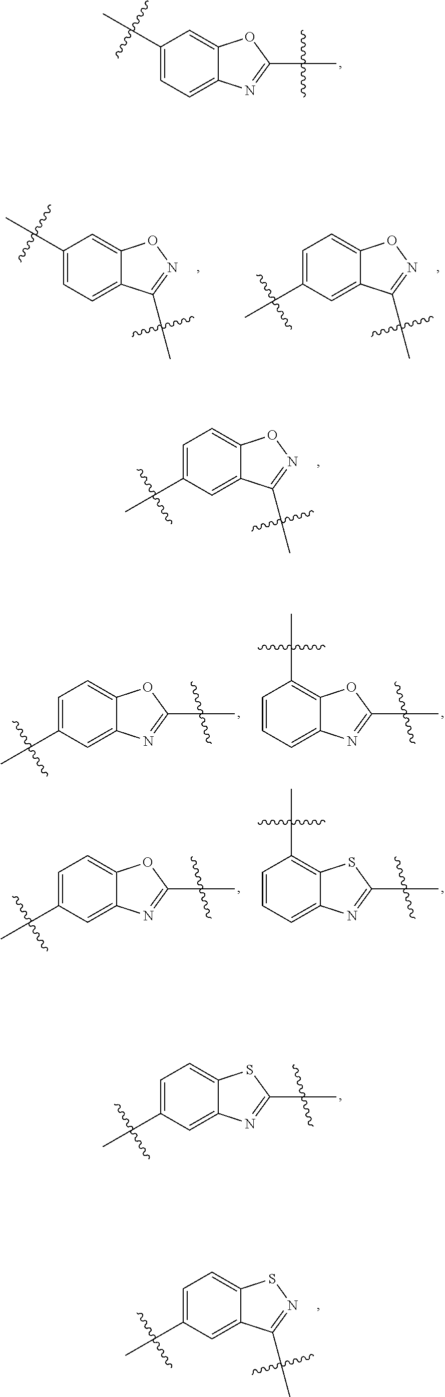

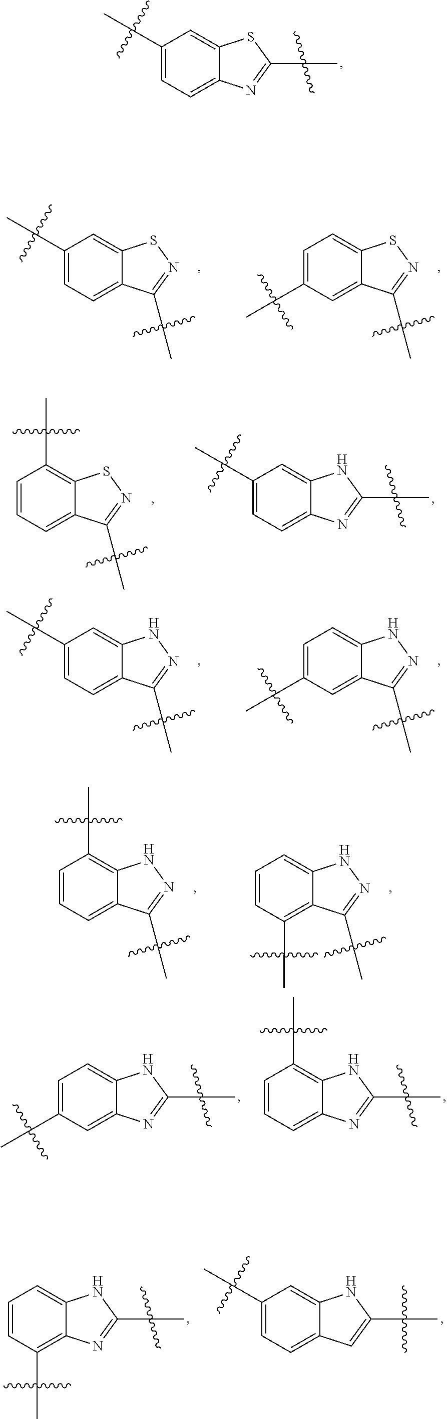

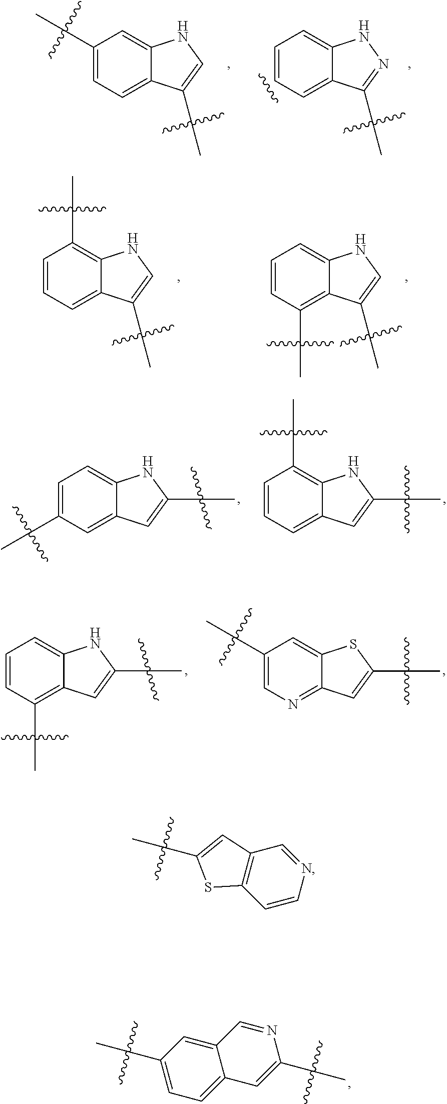

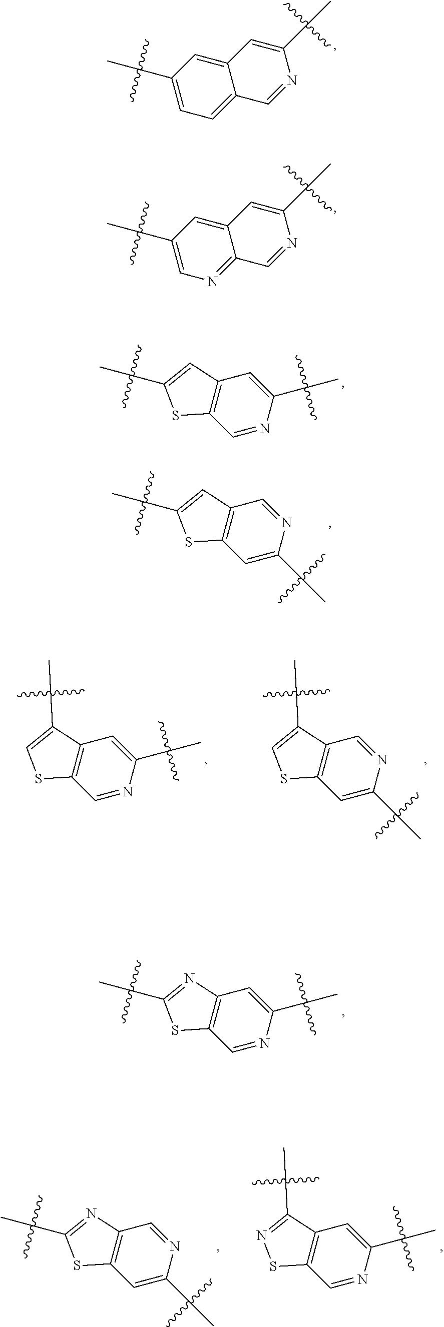

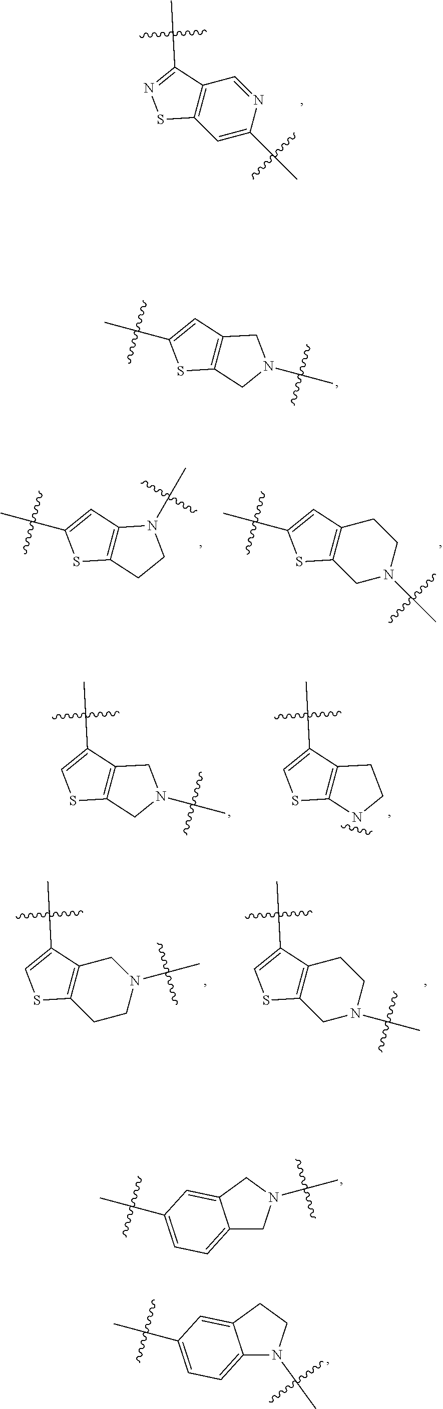

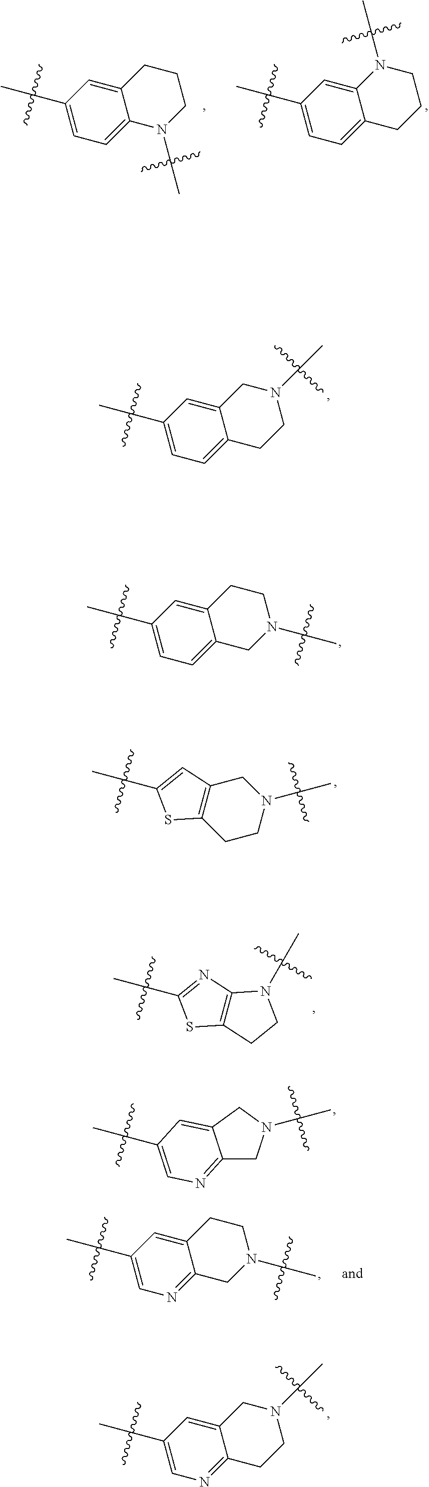

wherein, q=0, 1, 2, or 3; the groups described above are substituted with one or more R¹⁴, wherein R¹⁴ is located at any substitutable position in the groups, and R¹⁴, at each occurrence, is independently selected from hydrogen, halogen, hydroxyl, sulfhydryl, amino, cyano, formyl, acetyl, propionyl, methoxycarbonyl, ethoxycarbonyl, C₁₋₆ alkyl, C₁₋₆ haloalkyl, C₁₋₆ alkoxy, C₁₋₆ haloalkoxy, C₁₋₆ alkylamino, C₂₋₆ alkenyl, C₂₋₆ alkynyl, C₃₋₆ cycloalkyl, 4- to 6-membered heterocyclyl, C₆ aryl, and 5- to 6-membered heteroaryl;
preferably, R¹⁴, at each occurrence, is independently selected from hydrogen, F, Cl, Br, hydroxyl, amino, cyano, formyl, acetyl, propionyl, trifluoromethyl, difluoromethyl, —CH₂CF₃, —CH₂CHF₂, methyl, ethyl, n-propyl, isopropyl, cyclopropyl, n-butyl, isobutyl, tert-butyl, sec-butyl, methoxy, ethoxy, n-propoxy, isopropoxy, cyclopropoxy, n-butoxy, isobutoxy, tert-butoxy, sec-butoxy, trifluoromethoxy, —OCH₂CF₃, methylamino, ethylamino, n-propylamino, isopropylamino, cyclopropylamino, n-butylamino, isobutylamino, tert-butylamino, sec-butylamino, ethenyl, ethynyl, and phenyl.

In a preferred embodiment, L² is absent, or L² is selected from optionally substituted C₁₋₆ alkyl, optionally substituted C₃₋₁₂ cycloalkyl, optionally substituted 4- to 10-membered heterocyclyl, optionally substituted C₆₋₁₀ aryl, and optionally substituted 5- to 10-membered heteroaryl;
preferably, L² is absent, or L² is selected from optionally substituted C₁₋₄ alkyl, optionally substituted C₃₋₈ cycloalkyl, optionally substituted 4- to 10-membered heterocyclyl, optionally substituted C₆₋₈ aryl, and optionally substituted 5- to 6-membered heteroaryl;
more preferably, L² is absent, or L² is selected from optionally substituted C₁₋₄ alkyl, optionally substituted C₅₋₆ cycloalkyl, optionally substituted 5- to 6-membered heterocyclyl, optionally substituted C₆ aryl, and optionally substituted 5- to 6-membered heteroaryl;
the “optionally substituted” refers to being unsubstituted or being substituted with one or more R¹⁷; wherein, R¹⁷, at each occurrence, is independently selected from hydrogen, halogen, hydroxyl, sulfhydryl, nitro, amino, cyano, oxo, —R¹⁸, —OR¹⁸, —SR¹⁸, SO(R¹⁸), —SO₂(R¹⁸), —COOR¹⁸, —COR¹⁸, —NH(R¹⁸), —N(R¹⁸)(R¹⁹), —CONHR¹⁹, —CON(R¹⁸)(R¹⁹), —SONH(R¹⁸), —SON(R¹⁸)(R¹⁹), SO₂NH(R¹⁸), —SO₂N(R¹⁸)(R¹⁹), and —(CH₂)N(R¹⁸)(R¹⁹); wherein, R¹⁸ or R¹⁹, at each occurrence, is independently selected from hydrogen, methyl, trifluoromethyl, —CH₂CF₃, ethyl, n-propyl, isopropyl, n-butyl, isobutyl, tert-butyl, sec-butyl, cyclopropyl, cyclobutyl, 4- to 6-membered heterocyclyl, C₆ aryl, and 5- to 6-membered heteroaryl.

In a more preferred embodiment, L² is selected from the following groups optionally substituted with R¹⁷:
methyl, ethyl, n-propyl, isopropyl, n-butyl, isobutyl, tert-butyl, sec-butyl,

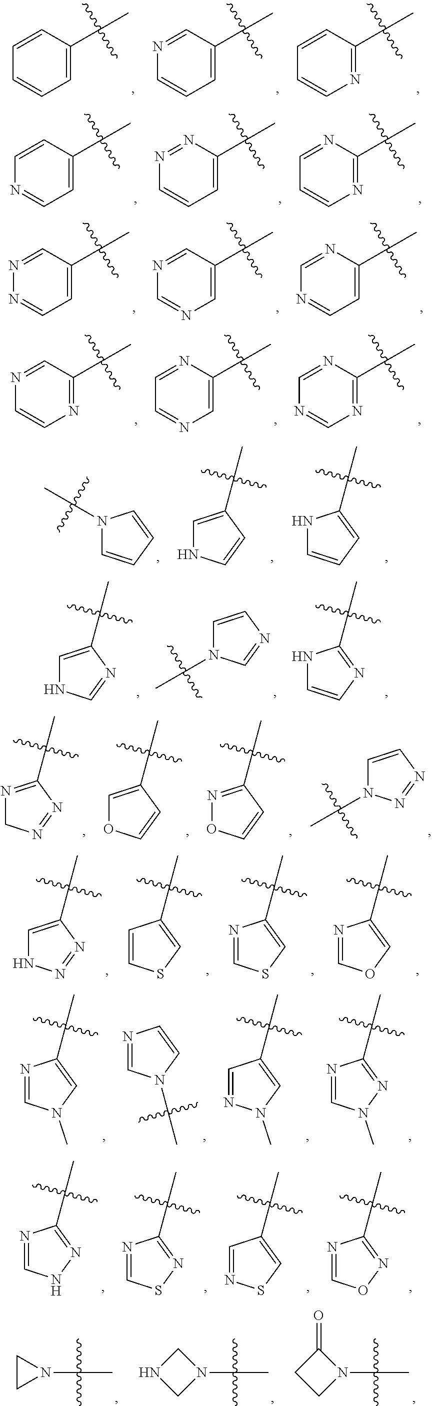

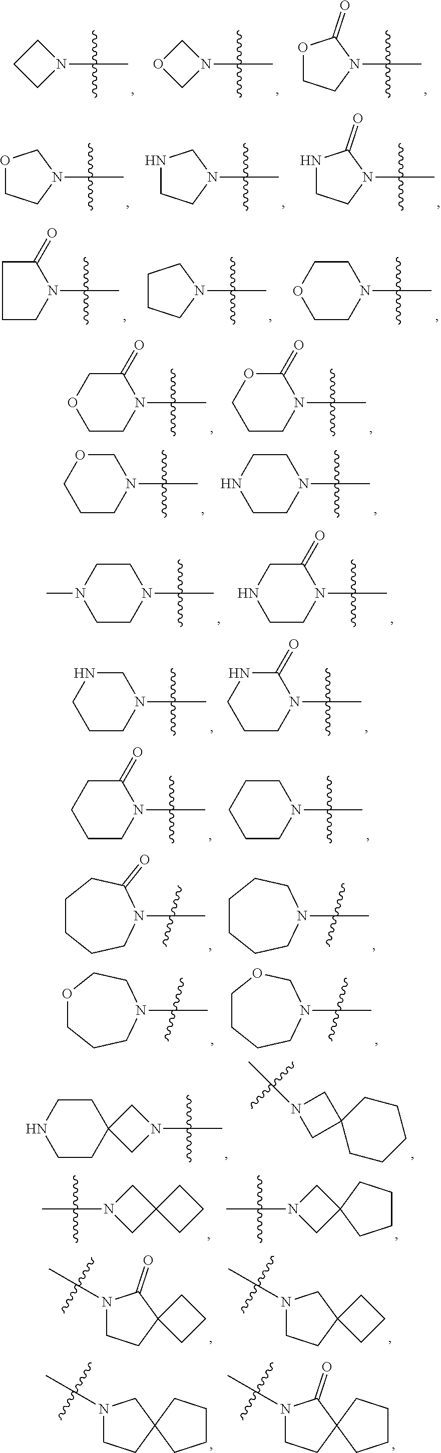

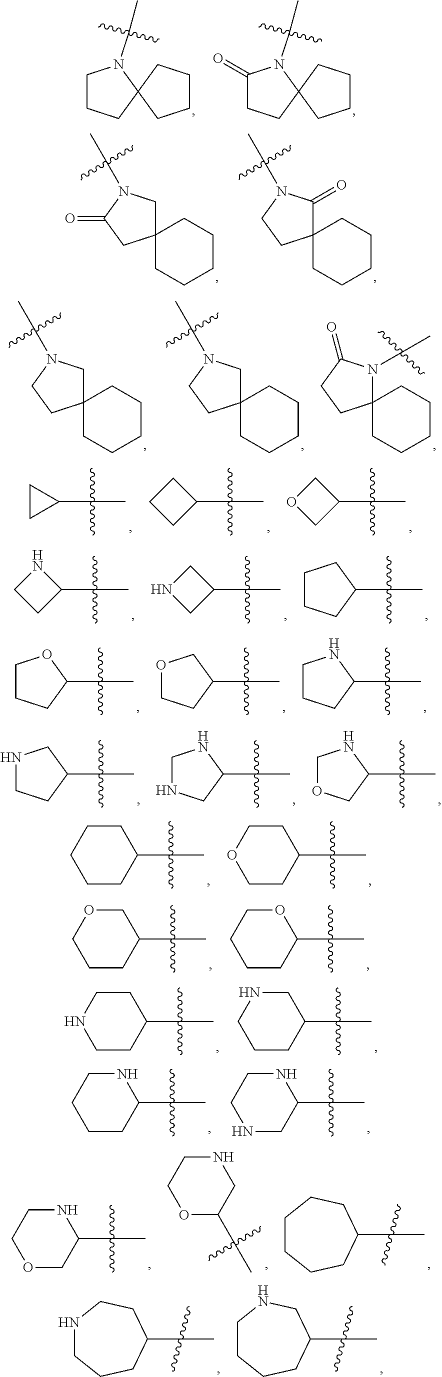

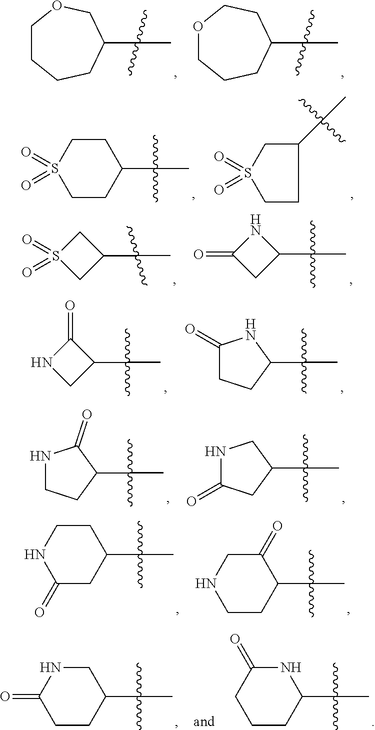

As a preferred embodiment, R¹⁷, at each occurrence, is independently selected from hydrogen, halogen, hydroxyl, sulfhydryl, amino, cyano, oxo, formyl, acetyl, propionyl, carboxyl, methoxycarbonyl, ethoxycarbonyl, C₁₋₆ alkyl, C₁₋₆ haloalkyl, C₁₋₆ alkoxy, C₁₋₆ haloalkoxy, C₁₋₆ alkylamino, —N(CH₃)₂, —N(Et)₂, —CONHCH₃, —CON(CH₃)(CH₃), —SO₂NH(CH₃), —SO₂N(CH₃)(CH₃), —(CH₂)NHCH₃, —(CH₂)NH(t-Bu), C₂₋₆ alkenyl, C₂₋₆ alkynyl, C₃₋₆ cycloalkyl, 4- to 6-membered heterocyclyl, phenyl, and 5- to 6-membered heteroaryl.

As a more preferred embodiment, R¹⁷, at each occurrence, is independently selected from hydrogen, F, Cl, Br, hydroxyl, amino, cyano, oxo, formyl, acetyl, propionyl, trifluoromethyl, —CH₂CF₃, methyl, ethyl, n-propyl, isopropyl, cyclopropyl, n-butyl, isobutyl, tert-butyl, sec-butyl, methoxy, ethoxy, n-propoxy, isopropoxy, cyclopropoxy, n-butoxy, isobutoxy, tert-butoxy, sec-butoxy, trifluoromethoxy, —OCH₂CF₃, methylamino, ethylamino, n-propylamino, isopropylamino, cyclopropylamino, n-butylamino, isobutylamino, tert-butylamino, sec-butylamino, —N(CH₃)₂, —N(Et)₂, —CONHCH₃, —CON(CH₃)(CH₃), —SO₂NH(CH₃), —SO₂N(CH₃)(CH₃), —(CH₂)NHCH₃, —(CH₂)NH(t-Bu), ethenyl, ethynyl, and phenyl.

The present disclosure further provides a compound represented by formula (IV), and a stereoisomer, an optical isomer, a pharmaceutically acceptable salt, a prodrug, and a solvate thereof:

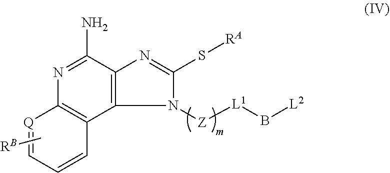

wherein, R^{A} is selected from optionally substituted C₁₋₆ alkyl, optionally substituted C₃₋₆ cycloalkyl, and optionally substituted —CH₂COOC₁₋₃ alkyl, the “optionally substituted” means being unsubstituted or being substituted with one or more substituents selected from hydroxyl, amino, nitro, carboxyl, cyano, acylamino, halogen, C₁₋₆ alkyl, C₃₋₆ cycloalkyl, C₂₋₆ alkenyl, C₂₋₆ alkynyl, and C₁₋₆ alkoxy;
R^{B} is selected from hydrogen, C₁₋₆ alkyl, hydroxyl, amino, carboxyl, cyano, nitro, halogen, C₁₋₆ alkoxy, and C₁₋₆ hydroxyalkyl;
Q is selected from C and N;
Z, at each occurrence, is independently selected from —O—, —S—, —C(R¹⁰)(R¹¹)—, —CO—, and —CS—; wherein, R¹⁰ or R¹¹, at each occurrence, is independently selected from hydrogen, halogen, hydroxyl, sulfhydryl, amino, cyano, C₁₋₆ alkyl, C₂₋₆ alkenyl, and C₂₋₆ alkynyl; m is selected from 0, 1, and 2;
B is selected from -(chemical bond), —O—, —S—, —N(R¹²)—, —CO—, and —C(R¹²)(R¹³)—; wherein, R¹² or R¹³, at each occurrence, is independently selected from hydrogen, deuterium, halogen, hydroxyl, sulfhydryl, amino, cyano, and C₁₋₆ alkyl;
L¹ is selected from optionally substituted C₁₋₁₂ alkyl, optionally substituted C₂₋₁₂ alkenyl, optionally substituted C₂₋₁₂ alkynyl, optionally substituted C₆₋₁₀ aryl, optionally substituted 5- to 10-membered heteroaryl, and optionally substituted 4- to 10-membered heterocyclyl; the “optionally substituted” refers to being unsubstituted or being substituted with one or more R¹⁴; wherein, R¹⁴ at each occurrence, is independently selected from halogen, hydroxyl, sulfhydryl, amino, nitro, cyano, C₁₋₆ alkyl, C₁₋₆ alkoxy, and C₃₋₆ cycloalkyl;
L² is selected from absence or L² is selected from optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted 4- to 10-membered heterocyclyl, optionally substituted C₆₋₁₀ aryl, optionally substituted 5- to 10-membered heteroaryl, optionally substituted —NHC₃₋₆ cycloalkyl, optionally substituted —NHC₁₋₆ alkyl, and optionally substituted —N(C₁₋₆ alkyl)₂, and preferably, L² is selected from absence or L² is selected from optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted 4- to 10-membered heterocyclyl, optionally substituted C₆₋₁₀ aryl, optionally substituted 5- to 10-membered heteroaryl, optionally substituted —NHC₁₋₆ alkyl, and optionally substituted —N(C₁₋₆ alkyl)₂; the “optionally substituted” refers to being unsubstituted or being substituted with one or more R¹⁷; wherein, R¹⁸, at each occurrence, is independently selected from hydrogen, halogen, hydroxyl, sulfhydryl, nitro, amino, cyano, oxo, —R¹⁸, —OR¹⁸, —SR¹⁸, —SO(R¹⁸), —SO₂(R¹⁸), —COOR¹⁸, —COR¹⁸, —NH(R¹⁸), —N(R¹⁸)(R¹⁹), —CONHR¹⁹, —CON(R¹⁸)(R¹⁹), —SONH(R¹⁸), —SON(R¹⁸)(R¹⁹), SO₂NH(R¹⁸), —SO₂N(R¹⁸)(R¹⁹), —C₀₋₃ alkyl-N(R¹⁸)(R¹⁹), —COC₁₋₃ alkyl-O(R¹⁸), and —C₁₋₃ alkyl-N(R¹⁸)(R¹⁹); wherein, the oxo means that two H at the same substitution site are substituted with the same O to form a divalent substituent ═O, and R¹⁸ or R¹⁹, at each occurrence, is independently selected from hydrogen, C₁₋₆ alkyl, C₁₋₆ haloalkyl, C₂₋₆ alkenyl, C₂₋₆ alkynyl, C₃₋₆ cycloalkyl, 4- to 6-membered heterocyclyl, aryl, and 5- to 6-membered heteroaryl.

The heteroatoms in the heterocyclyl and the heteroaryl are selected from N, O, and S, and the number of the heteroatoms is 1, 2, or 3.

In a preferred embodiment, R^{A} is selected from optionally substituted C₁₋₆ alkyl, optionally substituted C₃₋₆ cycloalkyl, and optionally substituted —CH₂COOC₁₋₃ alkyl, the “optionally substituted” means being unsubstituted or being substituted with one or more substituents selected from hydroxyl, amino, carboxyl, cyano, acylamino, halogen, methoxy, C₁₋₆ alkyl, C₃₋₆ cycloalkyl, and C₂₋₆ alkenyl;
preferably, R^{A} is selected from methyl, ethyl, n-propyl, isopropyl, n-butyl, isobutyl, tert-butyl, n-pentyl,

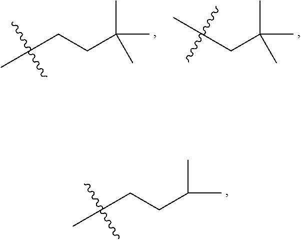

cyclopropyl, cyclobutyl, cyclopentyl, —CH₂COOCH₃, and —CH₂COOCH₂CH₃, the R^{A} being optionally substituted with one or more substituents selected from hydroxyl, amino, carboxyl, cyano, acylamino, halogen, methoxy, methyl, ethyl, ethenyl, cyclopropyl, cyclobutyl, and cyclopentyl;
more preferably, R^{A} is selected from methyl, ethyl, n-propyl, isopropyl, n-butyl, isobutyl, tert-butyl, n-pentyl,

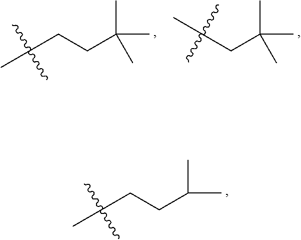

cyclopropyl, cyclobutyl, cyclopentyl, —CH₂COOCH₃, —CH₂COOCH₂CH₃, monofluoromethyl, difluoromethyl, trifluoromethyl, monofluoroethyl, difluoroethyl, trifluoroethyl, monofluoro-n-propyl, difluoro-n-propyl, trifluoro-n-propyl

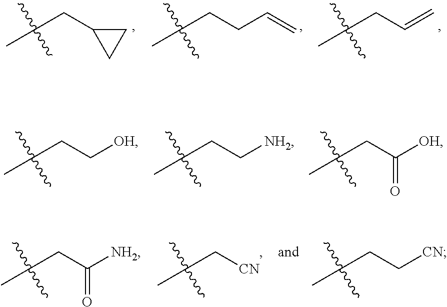

further preferably, R^{A} is selected from methyl, ethyl, n-propyl, n-butyl,

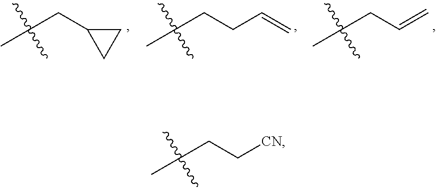

—CH₂CH₂F, —CH₂OCH₃, —CH₂CH(F)₂, and —CH₂CH₂C(F)₃;
still further preferably, R^{A} is selected from methyl.

In a preferred embodiment, R^{B} is selected from hydrogen, C₁₋₆ alkyl, halogen, and C₁₋₆ alkoxy; preferably, R^{B} is selected from hydrogen, methyl, ethyl, F, Cl, Br, methoxy, and ethoxy; more preferably, R^{B} is selected from hydrogen.

In a preferred embodiment, Q is selected from C.

In another preferred embodiment, Q is selected from N.

In a preferred embodiment, Z, at each occurrence, is independently selected from —O—, —S—, and —C(R¹⁰)(R¹¹)—; wherein, R¹⁰ or R¹¹, at each occurrence, is independently selected from hydrogen, halogen, hydroxyl, sulfhydryl, amino, cyano, methyl, and ethyl; m is selected from 0, 1, and 2;
preferably, Z, at each occurrence, is independently selected from —C(R¹⁰)(R¹¹)—; wherein, R¹⁰ or R¹¹, at each occurrence, is independently selected from hydrogen, halogen, hydroxyl, sulfhydryl, amino, cyano, methyl, and ethyl; m is selected from 1 and 2;
more preferably, Z, at each occurrence, is independently selected from —C(R¹⁰)(R¹¹)—; wherein, R¹⁰ or R¹¹, at each occurrence, is independently selected from hydrogen and methyl; m is selected from 1 and 2;
further preferably, Z, at each occurrence, is independently selected from —CH₂— and —CH(CH₃)—; m is selected from 1;
still further preferably, Z, at each occurrence, is independently selected from —CH₂—; m is selected from 1.

In a preferred embodiment, B is selected from -(chemical bond), —O—, —S—, —CO—, and —C(R¹²)(R¹³)—; wherein, R¹² or R¹³, at each occurrence, is independently selected from hydrogen, deuterium, halogen, hydroxyl, sulfhydryl, amino, cyano, and C₁₋₆ alkyl;
preferably, B is selected from -(chemical bond), —O—, and —C(R¹²)(R¹³)—; wherein, R¹² or R¹³, at each occurrence, is independently selected from hydrogen, methyl, and ethyl;
more preferably, B is selected from -(chemical bond), —O—, and —CH₂—; further preferably, B is selected from —CH₂—.

In a preferred embodiment, L¹ is selected from optionally substituted C₁₋₆ alkyl, optionally substituted C₆₋₁₀ aryl, optionally substituted 5- to 10-membered heteroaryl, and optionally substituted 4- to 10-membered heterocyclyl; the “optionally substituted” refers to being unsubstituted or being substituted with one or more R¹⁴; wherein, R¹⁴, at each occurrence, is independently selected from halogen, hydroxyl, sulfhydryl, amino, nitro, cyano, C₁₋₃ alkyl, and C₁₋₃ alkoxy;
preferably, L¹ is selected from optionally substituted C₁₋₆ alkyl, optionally substituted C₆₋₁₀ aryl, optionally substituted 5- to 10-membered heteroaryl, and optionally substituted 5- to 10-membered heterocyclyl; the “optionally substituted” refers to being unsubstituted or being substituted with one or more R¹⁴; wherein, R¹⁴, at each occurrence, is independently selected from halogen, C₁₋₃ alkyl, and C₁₋₃ alkoxy;
more preferably, L¹ is selected from optionally substituted C₁₋₆ alkyl, optionally substituted phenyl, optionally substituted 5-membered monocyclic heteroaryl, optionally substituted 6-membered monocyclic heteroaryl, optionally substituted 6-membered and 5-membered fused heteroaryl, optionally substituted 5-membered and 6-membered fused heteroaryl, optionally substituted 5-membered monocyclic heterocyclyl, optionally substituted 6-membered monocyclic heterocyclyl, optionally substituted 6-membered and 5-membered fused heterocyclyl, and optionally substituted 5-membered and 6-membered fused heterocyclyl; the “optionally substituted” refers to being unsubstituted or being substituted with one or more R¹⁴; wherein, R¹⁴, at each occurrence, is independently selected from halogen, methyl, ethyl, methoxy, and ethoxy;
further preferably, L¹ is selected from optionally substituted C₁₋₆ alkyl, optionally substituted phenyl, optionally substituted pyridinyl, and optionally substituted thienyl; the “optionally substituted” refers to being unsubstituted or being substituted with one or more R¹⁴; wherein, R¹⁴ at each occurrence, is independently selected from F, Cl, Br, methyl, and methoxy;
still further preferably, L¹ is selected from phenyl, pyridinyl, and thienyl, and is optionally substituted with F, Cl, methyl, and methoxy;
yet still further preferably, L¹ is selected from phenyl (e.g.,

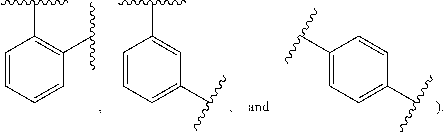

In a preferred embodiment, L² is absent, or L² is selected from optionally substituted C₁₋₆ alkyl, optionally substituted C₃₋₁₀ cycloalkyl, optionally substituted 4- to 10-membered heterocyclyl, optionally substituted C₆₋₁₀ aryl, and optionally substituted 5- to 10-membered heteroaryl; the “optionally substituted” refers to being unsubstituted or being substituted with one or more R¹⁷; wherein, R¹⁷, at each occurrence, is independently selected from hydrogen, halogen, hydroxyl, sulfhydryl, nitro, amino, cyano, oxo, —R¹⁸, —OR¹⁸, —COR¹⁸, —NH(R¹⁸), —N(R¹⁸)(R¹⁹) and —C₀₋₃ alkyl-N(R¹⁸)(R¹⁹); wherein, the oxo means that two H at the same substitution site are substituted with the same O to form a divalent substituent ═O, and R¹⁸ or R¹⁹, at each occurrence, is independently selected from hydrogen, C₁₋₆ alkyl, and C₃₋₆ cycloalkyl;
preferably, L² is selected from optionally substituted C₁₋₆ alkyl, optionally substituted cyclopropyl, optionally substituted cyclobutyl, optionally substituted cyclopentyl, optionally substituted cyclohexyl, optionally substituted 4-membered monocyclic heterocycloalkyl, optionally substituted 5-membered monocyclic heterocycloalkyl, optionally substituted 6-membered monocyclic heterocycloalkyl, optionally substituted 4-membered and 4-membered fused heterocycloalkyl, optionally substituted 4-membered and 5-membered fused heterocycloalkyl, optionally substituted 5-membered and 4-membered fused heterocycloalkyl, optionally substituted 5-membered and 5-membered fused heterocycloalkyl, optionally substituted 5-membered and 6-membered fused heterocycloalkyl, optionally substituted 6-membered and 5-membered fused heterocycloalkyl, optionally substituted 5-membered monocyclic heteroaryl, and optionally substituted 6-membered monocyclic heteroaryl; the “optionally substituted” refers to being unsubstituted or being substituted with one or more R¹⁷; wherein, R¹⁷, at each occurrence, is independently selected from hydrogen, halogen, hydroxyl, amino, —R¹⁸, —OR¹⁸, —N(R¹⁸)(R¹⁹), and —C₀₋₃ alkyl-N(R¹⁸)(R¹⁹); wherein, R¹⁸ or R¹⁹, at each occurrence, is independently selected from hydrogen, C₁₋₆ alkyl, and C₃₋₆ cycloalkyl;
more preferably, L² is selected from optionally substituted C₁₋₆ alkyl, optionally substituted 4-membered monocyclic heterocycloalkyl, optionally substituted 5-membered monocyclic heterocycloalkyl, optionally substituted 6-membered monocyclic heterocycloalkyl, optionally substituted 5-membered and 5-membered fused heterocycloalkyl, optionally substituted 5-membered monocyclic heteroaryl, and optionally substituted 6-membered monocyclic heteroaryl;
the “optionally substituted” refers to being unsubstituted or being substituted with one or more R¹⁷; wherein, R¹⁷, at each occurrence, is independently selected from hydrogen, F, Cl, Br, hydroxyl, amino, methyl, ethyl, methylamino, ethylamino, n-propylamino, isopropylamino, dimethylamino, diethylamino, cyclopropylamino, cyclobutylamino, cyclopentylamino, cyclobutylaminomethyl, and cyclobutylaminoethyl;
further preferably, L² is selected from optionally substituted methyl, optionally substituted ethyl, optionally substituted propyl, optionally substituted pyrrolidinyl, optionally substituted azetidinyl, optionally substituted pyrazolyl, optionally substituted piperazinyl, optionally substituted piperidyl, and optionally substituted

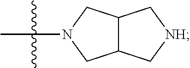 [SMILES: *N1CC2CNCC2C1]

the “optionally substituted” refers to being unsubstituted or being substituted with one or more R¹⁷; wherein, R¹⁷, at each occurrence, is independently selected from hydrogen, F, Cl, Br, hydroxyl, amino, methyl, ethyl, methylamino, ethylamino, n-propylamino, isopropylamino, dimethylamino, cyclopropylamino, cyclobutylamino, cyclopentylamino, cyclobutylaminomethyl, and cyclobutylaminoethyl;
still further preferably, L² is selected from

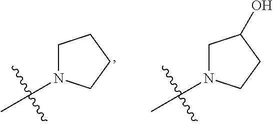 [SMILES: *N1CCCC1 ; *N1CCC(O)C1]

(including both the R configuration and the S configuration),

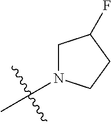 [SMILES: *N1CCC(F)C1]

(including both the R configuration and the S configuration),

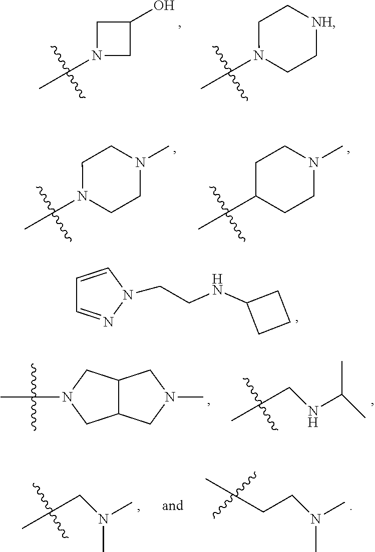

The present disclosure further provides a compound represented by formula (V), and a stereoisomer, an optical isomer, a pharmaceutically acceptable salt, a prodrug, and a solvate thereof:

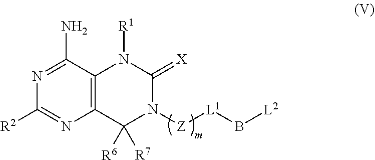

wherein, X is selected from O and S;
R¹ is selected from hydrogen, hydroxyl, amino, C₁₋₆ alkyl, C₃₋₆ cycloalkyl, 4- to 6-membered heterocyclyl, aryl, and 5- to 6-membered heteroaryl;
R² is selected from hydrogen, halogen, hydroxyl, sulfhydryl, amino, cyano, C₁₋₆ alkyl, C₁₋₆ haloalkyl, C₁₋₆ alkoxy, C₁₋₆ alkylthio, C₁₋₆ alkylsulfinyl, C₁₋₆ alkylsulfinylimino, C₂₋₆ alkenyl, C₂₋₆ alkynyl, C₃₋₆ cycloalkyl, 4- to 6-membered heterocyclyl, aryl, and 5- to 6-membered heteroaryl, and preferably, R² is selected from hydrogen, halogen, hydroxyl, sulfhydryl, amino, cyano, C₁₋₆ alkyl, C₁₋₆ haloalkyl, C₁₋₆ alkoxy, C₂₋₆ alkenyl, C₂₋₆ alkynyl, C₃₋₆ cycloalkyl, 4- to 6-membered heterocyclyl, aryl, and 5- to 6-membered heteroaryl;
R⁶ or R⁷, at each occurrence, is independently selected from hydrogen, halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl;
Z, at each occurrence, is independently selected from —O—, —S—, —C(R¹⁰)(R¹¹)—, —CO—, —CS—, —CO₂—, and —N(R¹⁰)—; wherein, R¹⁰ or R¹¹, at each occurrence, is independently selected from hydrogen, halogen, hydroxyl, sulfhydryl, amino, cyano, C₁₋₆ alkyl, C₂₋₆ alkenyl, and C₂₋₆ alkynyl; m is 1 or 2;
B is selected from -(chemical bond), —O—, —S—, —N(R¹²)—, —CO—, —C(R¹²)(R¹³)—, and —C(R¹²)(R¹³)—C(R¹²)(R¹³)—; wherein, R¹² or R¹³, at each occurrence, is independently selected from hydrogen, deuterium, halogen, hydroxyl, sulfhydryl, amino, cyano, C₁₋₆ alkyl, C₁₋₆ alkoxy, C₁₋₆ haloalkyl, C₂₋₆ alkenyl, C₂₋₆ alkynyl, and C₃₋₆ cycloalkyl;
L¹ is selected from optionally substituted C₁₋₁₂ alkyl, optionally substituted C₆₋₁₀ aryl, optionally substituted 5- to 10-membered heteroaryl, optionally substituted C₃₋₇ cycloalkyl, and optionally substituted 4- to 10-membered heterocyclyl; the “optionally substituted” refers to being unsubstituted or being substituted with one or more R¹⁴; wherein, R¹⁴, at each occurrence, is independently selected from hydrogen, halogen, hydroxyl, sulfhydryl, amino, nitro, cyano, C₁₋₆ alkyl, C₁₋₆ alkoxy, C₁₋₆ haloalkyl, C₂₋₆ alkenyl, and C₂₋₆ alkynyl;
L² is absent, or L² is selected from optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted 4- to 10-membered heterocyclyl, optionally substituted C₆₋₁₀ aryl, and optionally substituted 5- to 10-membered heteroaryl; the “optionally substituted” refers to being unsubstituted or being substituted with one or more R¹⁷; wherein, R¹⁷, at each occurrence, is independently selected from hydrogen, halogen, carboxyl, hydroxyl, sulfhydryl, nitro, amino, cyano, oxo, C₁₋₆ alkyl, C₁₋₆ alkoxy, C₁₋₆ haloalkyl, C₂₋₆ alkenyl, C₂₋₆ alkynyl, —NHC₁₋₆ alkyl, —N(C₁₋₆ alkyl)₂, —C₁₋₆ alkyl-NHC₁₋₆ alkyl, C₃₋₆ cycloalkyl, 4- to 6-membered heterocyclyl, aryl, and 5- to 6-membered heteroaryl, and preferably, R¹⁷, at each occurrence, is independently selected from hydrogen, halogen, hydroxyl, sulfhydryl, nitro, amino, cyano, oxo, C₁₋₆ alkyl, C₁₋₆ alkoxy, C₁₋₆ haloalkyl, C₂₋₆ alkenyl, C₂₋₆ alkynyl, —NHC₁₋₆ alkyl, —N(C₁₋₆ alkyl)₂, —C₁₋₆ alkyl-NHC₁₋₆ alkyl, C₃₋₆ cycloalkyl, 4- to 6-membered heterocyclyl, aryl, and 5- to 6-membered heteroaryl.

In a preferred embodiment, X is O.

In a preferred embodiment, R¹ is selected from hydrogen and C₁₋₆ alkyl;
preferably, R¹ is selected from hydrogen, methyl, and ethyl;
more preferably, R¹ is selected from hydrogen.

In a preferred embodiment, R² is selected from hydrogen, halogen, hydroxyl, sulfhydryl, amino, cyano, C₁₋₆ alkyl, C₁₋₆ haloalkyl, and C₁₋₆ alkoxy;
preferably, R² is selected from hydrogen, halogen, hydroxyl, amino, cyano, methyl, ethyl, n-propyl, isopropyl, monofluoromethyl, monofluoroethyl, difluoromethyl, difluoroethyl, trifluoromethyl, trifluoroethyl, methoxy, ethoxy, n-propoxy, isopropoxy, n-butoxy, and n-pentoxy;
more preferably, R² is selected from methoxy, ethoxy, n-propoxy, isopropoxy, and n-butoxy;
further preferably, R² is selected from n-butoxy.

In a preferred embodiment, R⁶ or R⁷, at each occurrence, is independently selected from hydrogen, halogen, hydroxyl, amino, cyano, and C₁₋₆ alkyl;
preferably, R⁶ or R⁷, at each occurrence, is independently selected from hydrogen, halogen, methyl, and ethyl;
more preferably, R⁶ or R⁷, at each occurrence, is independently selected from hydrogen and methyl;
further preferably, R⁶ or R⁷, at each occurrence, is independently selected from hydrogen.

In a preferred embodiment, Z, at each occurrence, is independently selected from —O—, —C(R¹⁰)(R¹¹)—, and —CO—; wherein, R¹⁰ or R¹¹, at each occurrence, is independently selected from hydrogen, halogen, hydroxyl, sulfhydryl, amino, cyano, C₁₋₆ alkyl, C₂₋₆ alkenyl, and C₂₋₆ alkynyl;
m is 1 or 2;
preferably, Z, at each occurrence, is independently selected from —C(R¹⁰)(R¹¹)—; wherein, R¹⁰ or R¹¹, at each occurrence, is independently selected from hydrogen, halogen, hydroxyl, and C₁₋₆ alkyl; m is 1 or 2;
more preferably, Z, at each occurrence, is independently selected from —C(R¹⁰)(R¹¹)—; wherein, R¹⁰ or R¹¹, at each occurrence, is independently selected from hydrogen, methyl, and ethyl; m is 1;
further preferably, Z, at each occurrence, is independently selected from —CH₂— and —CH(CH₃)—; m is 1;
still further preferably, Z, at each occurrence, is independently selected from —CH₂—; m is 1.

In a preferred embodiment, B is selected from -(chemical bond), —O—, —CO—, and —C(R¹²)(R¹³)—; wherein, R¹² or R¹³, at each occurrence, is independently selected from hydrogen, deuterium, halogen, hydroxyl, sulfhydryl, amino, cyano, and C₁₋₆ alkyl;
preferably, B is selected from —CO—, —O—, —CH₂—, —CH(CH₃)—, and -CD₂-;
more preferably, B is selected from —CO—, —O—, and —CH₂—.

In a preferred embodiment, L¹ is selected from optionally substituted C₆₋₁₀ aryl and optionally substituted 5- to 10-membered heteroaryl; the “optionally substituted” refers to being unsubstituted or being substituted with one or more R¹⁴; wherein, R¹⁴, at each occurrence, is independently selected from hydrogen, halogen, hydroxyl, sulfhydryl, amino, nitro, cyano, C₁₋₆ alkyl, C₁₋₆ alkoxy, and C₁₋₆ haloalkyl;
preferably, L¹ is selected from optionally substituted C₆₋₁₀ aryl and optionally substituted 5- to 10-membered heteroaryl; the “optionally substituted” refers to being unsubstituted or being substituted with one or more R¹⁴; wherein, R¹⁴, at each occurrence, is independently selected from hydrogen, halogen, C₁₋₆ alkyl, C₁₋₆ alkoxy, and C₁₋₆ haloalkyl;
more preferably, L¹ is selected from optionally substituted phenyl, optionally substituted pyridinyl, optionally substituted thienyl, and optionally substituted furanyl; the “optionally substituted” refers to being unsubstituted or being substituted with one or more R¹⁴; wherein, R¹⁴, at each occurrence, is independently selected from hydrogen, F, Cl, Br, methyl, ethyl, methoxy, and ethoxy;
further preferably, L¹ is selected from optionally substituted phenyl, optionally substituted pyridinyl, optionally substituted thienyl, and optionally substituted furanyl; the “optionally substituted” refers to being unsubstituted or being substituted with one or more R¹⁴; wherein, R¹⁴ at each occurrence, is independently selected from hydrogen and methyl;
still further preferably, L¹ is selected from phenyl, methyl-substituted phenyl, pyridinyl, methyl-substituted pyridinyl, thienyl, and methyl-substituted thienyl;
yet still further preferably, L¹ is selected from phenyl (e.g.,

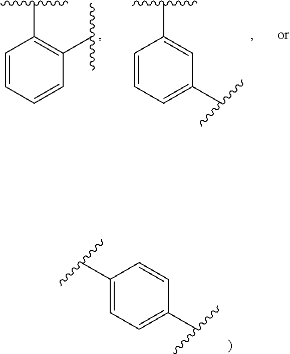

and monomethyl-substituted phenyl (e.g.,

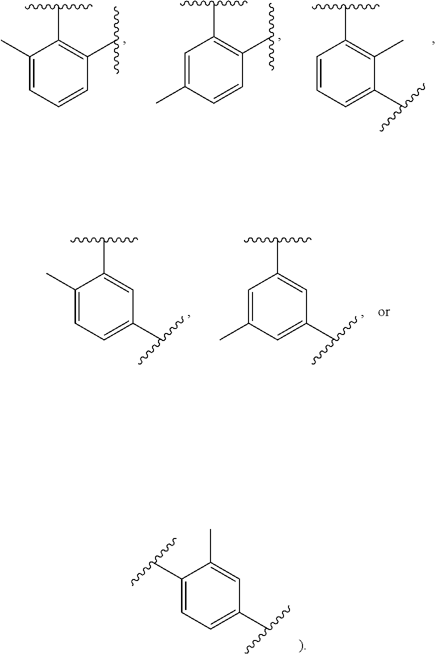

In a preferred embodiment of the present disclosure, L² is selected from optionally substituted C₃₋₁₀ cycloalkyl, optionally substituted 4- to 10-membered heterocyclyl, optionally substituted C₆₋₁₀ aryl, and optionally substituted 5- to 10-membered heteroaryl; the “optionally substituted” is being unsubstituted or being substituted with one or more R¹⁷; wherein, R¹⁷, at each occurrence, is independently selected from hydrogen, halogen, hydroxyl, sulfhydryl, nitro, amino, cyano, oxo, C₁₋₆ alkyl, —C₁₋₃ alkyl-NHC₁₋₃ alkyl, C₁₋₆ alkoxy, and C₁₋₆ haloalkyl;
preferably, L² is selected from optionally substituted 4-membered monocyclic heterocyclyl, optionally substituted 5-membered monocyclic heterocyclyl, optionally substituted 6-membered monocyclic heterocyclyl, optionally substituted phenyl, optionally substituted 5-membered monocyclic heteroaryl, and optionally substituted 6-membered monocyclic heteroaryl; the “optionally substituted” refers to being unsubstituted or being substituted with one or more R¹⁷; wherein, R¹⁷, at each occurrence, is independently selected from hydrogen, halogen, hydroxyl, nitro, amino, cyano, oxo, methyl, ethyl, n-propyl, isopropyl, methoxy, ethoxy, and —CH₂—NHCH₃; more preferably, L² is selected from optionally substituted 4-membered monocyclic heterocycloalkyl, optionally substituted 5-membered monocyclic heterocycloalkyl, optionally substituted 6-membered monocyclic heterocycloalkyl, optionally substituted 5-membered monocyclic heteroaryl, optionally substituted 6-membered monocyclic heteroaryl, and optionally substituted phenyl; the “optionally substituted” refers to being unsubstituted or being substituted with one or more R¹⁷; wherein, R¹⁷, at each occurrence, is independently selected from hydrogen, halogen, oxo, methyl, ethyl, methoxy, ethoxy, and —CH₂—NHCH₃;
further preferably, L² is selected from optionally substituted 5-membered monocyclic heterocycloalkyl, optionally substituted 5-membered monocyclic heteroaryl, and optionally substituted phenyl; the “optionally substituted” refers to being unsubstituted or being substituted with one or more R¹⁷; wherein, R¹⁷, at each occurrence, is independently selected from hydrogen, halogen, oxo, methyl, ethyl, methoxy, ethoxy, and —CH₂—NHCH₃;
still further preferably, L² is selected from optionally substituted pyrrolyl, optionally substituted phenyl, and optionally substituted imidazolyl; alternatively, L² is selected from optionally substituted pyrrolidinyl, optionally substituted phenyl, and optionally substituted imidazolyl; the “optionally substituted” refers to being unsubstituted or being substituted with one or more R¹⁷; wherein, R¹⁷, at each occurrence, is independently selected from hydrogen, halogen, oxo, methyl, ethyl, methoxy, ethoxy, and —CH₂—NHCH₃;
yet still further preferably, L² is selected from optionally substituted pyrrolyl, optionally substituted phenyl, and optionally substituted imidazolyl; alternatively, L² is selected from optionally substituted pyrrolidinyl, optionally substituted phenyl, and optionally substituted imidazolyl; the “optionally substituted” refers to being unsubstituted or being substituted with one or more R¹⁷; wherein, R¹⁷, at each occurrence, is independently selected from hydrogen, F, Cl, Br, methyl, methoxy, and —CH₂—NHCH₃;
yet still further more preferably, L² is selected from pyrrolyl, imidazolyl, and —CH₂—NHCH₃ substituted phenyl; alternatively, L² is selected from pyrrolidinyl (e.g.,

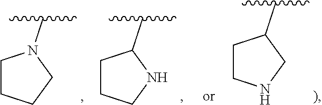 [SMILES: *N1CCCC1 ; *C1CCCN1 ; *C1CCNC1]

imidazolyl, and —CH₂—NHCH₃ substituted phenyl.

The present disclosure further provides a compound represented by formula (VI), and a stereoisomer, an optical isomer, a pharmaceutically acceptable salt, a prodrug, and a solvate thereof:

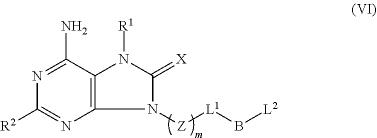

wherein, X is O or S;
R¹ is selected from hydrogen, hydroxyl, amino, C₁₋₆ alkyl, C₃₋₆ cycloalkyl, 4- to 6-membered heterocyclyl, aryl, and 5- to 6-membered heteroaryl;
R² is selected from hydrogen, halogen, hydroxyl, sulfhydryl, amino, cyano, C₁₋₆ alkyl, C₁₋₆ haloalkyl, C₁₋₆ alkoxy, C₂₋₆ alkenyl, C₂₋₆ alkynyl, C₃₋₆ cycloalkyl, 4- to 6-membered heterocyclyl, aryl, and 5- to 6-membered heteroaryl;
Z, at each occurrence, is independently selected from —O—, —S—, —C(R¹⁰)(R¹¹)—, —CO—, —CS—, —CO₂—, and —N(R¹⁰)—; wherein, R¹⁰ or R¹¹, at each occurrence, is independently selected from hydrogen, halogen, hydroxyl, sulfhydryl, amino, cyano, C₁₋₆ alkyl, C₂₋₆ alkenyl, and C₂₋₆ alkynyl; m is 1 or 2;
B is selected from -(chemical bond), —O—, —S—, —N(R¹²)—, —CO—, —C(R¹²)(R¹³)—, and —C(R¹²)(R¹³)—C(R¹²)(R¹³)—; wherein, R¹² or R¹³, at each occurrence, is independently selected from hydrogen, deuterium, halogen, hydroxyl, sulfhydryl, amino, cyano, C₁₋₆ alkyl, C₁₋₆ alkoxy, C₁₋₆ haloalkyl, C₂₋₆ alkenyl, C₂₋₆ alkynyl, and C₃₋₆ cycloalkyl;
L¹ is selected from optionally substituted C₁₋₁₂ alkyl, optionally substituted C₆₋₁₀ aryl, optionally substituted 5- to 10-membered heteroaryl, optionally substituted C₃₋₇ cycloalkyl, and optionally substituted 4- to 10-membered heterocyclyl; the “optionally substituted” refers to being unsubstituted or being substituted with one or more R¹⁴; wherein, R¹⁴, at each occurrence, is independently selected from hydrogen, halogen, hydroxyl, sulfhydryl, amino, nitro, cyano, C₁₋₆ alkyl, C₁₋₆ alkoxy, C₁₋₆ haloalkyl, C₂₋₆ alkenyl, and C₂₋₆ alkynyl;
L² is absent, or L² is selected from optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted 4- to 10-membered heterocyclyl, optionally substituted C₆₋₁₀ aryl, and optionally substituted 5- to 10-membered heteroaryl; the “optionally substituted” refers to being unsubstituted or being substituted with one or more R¹⁷; wherein, R¹⁷, at each occurrence, is independently selected from hydrogen, halogen, hydroxyl, sulfhydryl, nitro, amino, cyano, oxo, C₁₋₆ alkyl, C₁₋₆ alkoxy, C₁₋₆ haloalkyl, C₂₋₆ alkenyl, C₂₋₆ alkynyl, —NHC₁₋₆ alkyl, —N(C₁₋₆ alkyl)₂, —COC₁₋₃ alkyl-NH₂, —COC₁₋₃ alkyl-OC₁₋₃ alkyl, —COC₁₋₃ alkyl-OH, C₃₋₆ cycloalkyl, 4- to 6-membered heterocyclyl, aryl, and 5- to 6-membered heteroaryl.

In a preferred embodiment, X is O.

In a preferred embodiment of the present disclosure, R¹ is selected from hydrogen and C₁₋₆ alkyl;
preferably, R¹ is selected from hydrogen, methyl, and ethyl;
more preferably, R¹ is selected from hydrogen.

In a preferred embodiment, R² is selected from hydrogen, halogen, hydroxyl, sulfhydryl, amino, cyano, C₁₋₆ alkyl, C₁₋₆ haloalkyl, and C₁₋₆ alkoxy;
preferably, R² is selected from hydrogen, halogen, hydroxyl, amino, cyano, methyl, ethyl, n-propyl, isopropyl, monofluoromethyl, monofluoroethyl, difluoromethyl, difluoroethyl, trifluoromethyl, trifluoroethyl, methoxy, ethoxy, n-propoxy, isopropoxy, n-butoxy, and n-pentoxy;
more preferably, R² is selected from methoxy, ethoxy, n-propoxy, isopropoxy, and n-butoxy;
further preferably, R² is selected from n-butoxy.

In a preferred embodiment, Z, at each occurrence, is independently selected from —O—, —C(R¹⁰)(R¹¹)—, and —CO—; wherein, R¹⁰ or R¹¹, at each occurrence, is independently selected from hydrogen, halogen, hydroxyl, sulfhydryl, amino, cyano, C₁₋₆ alkyl, C₂₋₆ alkenyl, and C₂₋₆ alkynyl;
m is 1 or 2;
preferably, Z, at each occurrence, is independently selected from —C(R¹⁰)(R¹¹)—; wherein, R¹⁰ or R¹¹, at each occurrence, is independently selected from hydrogen, halogen, hydroxyl, and C₁₋₆ alkyl; m is 1 or 2;
more preferably, Z, at each occurrence, is independently selected from —C(R¹⁰)(R¹¹)—; wherein, R¹⁰ or R¹¹, at each occurrence, is independently selected from hydrogen, methyl, and ethyl; m is 1;
further preferably, Z, at each occurrence, is independently selected from —CH₂— and —CH(CH₃)—; m is 1;
still further preferably, Z, at each occurrence, is independently selected from —CH₂—; m is 1.

In a preferred embodiment, B is selected from -(chemical bond), —O—, —CO—, and —C(R¹²)(R¹³)—; wherein, R¹² or R¹³, at each occurrence, is independently selected from hydrogen, deuterium, halogen, hydroxyl, sulfhydryl, amino, cyano, and C₁₋₆ alkyl;
preferably, B is selected from —CO—, —CH₂—, —CH(CH₃)—, and -CD₂-;
more preferably, B is selected from —CO— and —CH₂—;
further preferably, B is selected from —CH₂—.

In a preferred embodiment, L¹ is selected from optionally substituted C₆₋₁₀ aryl and optionally substituted 5- to 10-membered heteroaryl; the “optionally substituted” refers to being unsubstituted or being substituted with one or more R¹⁴; wherein, R¹⁴, at each occurrence, is independently selected from hydrogen, halogen, hydroxyl, sulfhydryl, amino, nitro, cyano, C₁₋₆ alkyl, C₁₋₆ alkoxy, and C₁₋₆ haloalkyl;
preferably, L¹ is selected from optionally substituted C₆₋₁₀ aryl and optionally substituted 5- to 10-membered heteroaryl; the “optionally substituted” refers to being unsubstituted or being substituted with one or more R¹⁴; wherein, R¹⁴, at each occurrence, is independently selected from hydrogen, halogen, C₁₋₆ alkyl, C₁₋₆ alkoxy, and C₁₋₆ haloalkyl;
more preferably, L¹ is selected from optionally substituted phenyl, optionally substituted pyridinyl, optionally substituted thienyl, and optionally substituted furanyl; the “optionally substituted” refers to being unsubstituted or being substituted with one or more R¹⁴; wherein, R¹⁴, at each occurrence, is independently selected from hydrogen, F, Cl, Br, methyl, ethyl, methoxy, and ethoxy;
further preferably, L¹ is selected from optionally substituted phenyl, optionally substituted thienyl, and optionally substituted furanyl; the “optionally substituted” refers to being unsubstituted or being substituted with one or more R¹⁴; wherein, R¹⁴, at each occurrence, is independently selected from hydrogen and methyl;
still further preferably, L¹ is selected from phenyl, thienyl, and furanyl;
yet still further preferably, L¹ is selected from phenyl (e.g.,

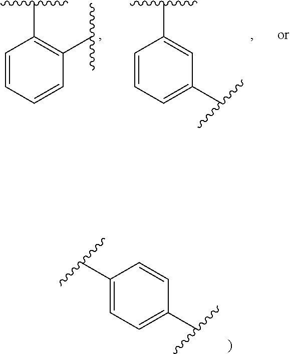

and thienyl (e.g.,

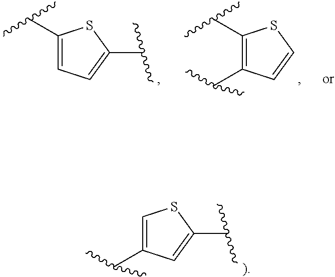 [SMILES: *c1ccc(*)s1 ; *c1ccsc1* ; *c1csc(*)c1]

In a preferred embodiment of the present disclosure, L² is selected from optionally substituted C₃₋₁₀ cycloalkyl, optionally substituted 4- to 10-membered heterocyclyl, optionally substituted C₆₋₁₀ aryl, and optionally substituted 5- to 10-membered heteroaryl; the “optionally substituted” refers to being unsubstituted or being substituted with one or more R¹⁷; wherein, R¹⁷, at each occurrence, is independently selected from hydrogen, halogen, hydroxyl, sulfhydryl, nitro, amino, cyano, oxo, C₁₋₆ alkyl, C₁₋₆ alkoxy, C₁₋₆ haloalkyl, —COC₁₋₃ alkyl-NH₂, —COC₁₋₃ alkyl-OC₁₋₃ alkyl, and —COC₁₋₃ alkyl-OH;
preferably, L² is selected from optionally substituted 4-membered monocyclic heterocyclyl, optionally substituted 5-membered monocyclic heterocyclyl, optionally substituted 6-membered monocyclic heterocyclyl, optionally substituted phenyl, optionally substituted 5-membered monocyclic heteroaryl, and optionally substituted 6-membered monocyclic heteroaryl; the “optionally substituted” refers to being unsubstituted or being substituted with one or more R¹⁷; wherein, R¹⁷, at each occurrence, is independently selected from hydrogen, halogen, hydroxyl, nitro, amino, cyano, oxo, methyl, ethyl, n-propyl, isopropyl, methoxy, ethoxy, —COCH₂NH₂, —COCH₂OCH₃, and —COCH₂OH;
more preferably, L² is selected from optionally substituted 4-membered monocyclic heterocycloalkyl, optionally substituted 5-membered monocyclic heterocycloalkyl, optionally substituted 6-membered monocyclic heterocycloalkyl, optionally substituted 5-membered monocyclic heteroaryl, and optionally substituted 6-membered monocyclic heteroaryl; the “optionally substituted” refers to being unsubstituted or being substituted with one or more R¹⁷; wherein, R¹⁷, at each occurrence, is independently selected from hydrogen, halogen, hydroxyl, amino, oxo, methyl, ethyl, methoxy, ethoxy, —COCH₂NH₂, —COCH₂OCH₃, and —COCH₂OH;
further preferably, L² is selected from optionally substituted 5-membered monoheterocycloalkyl and optionally substituted 6-membered monoheterocycloalkyl; the “optionally substituted” refers to being unsubstituted or being substituted with one or more R¹⁷; wherein, R¹⁷, at each occurrence, is independently selected from hydrogen, halogen, hydroxyl, amino, oxo, methyl, methoxy, —COCH₂NH₂, —COCH₂OCH₃, and —COCH₂OH;
still further preferably, L² is selected from optionally substituted pyrrolyl and optionally substituted piperazinyl; alternatively, L² is selected from optionally substituted pyrrolidinyl and optionally substituted piperazinyl; the “optionally substituted” refers to being unsubstituted or being substituted with one or more R¹⁷; wherein, R¹⁷, at each occurrence, is independently selected from hydrogen, hydroxyl, amino, oxo, methyl, methoxy, —COCH₂NH₂, —COCH₂OCH₃, and —COCH₂OH;
yet still further preferably, L² is selected from pyrrolyl, piperazinyl,

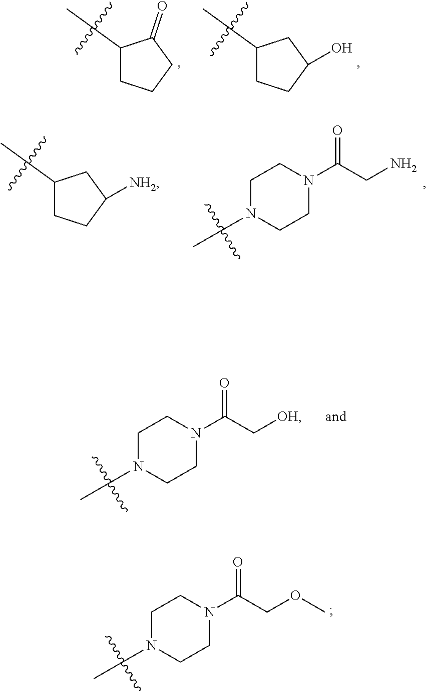

alternatively, L² is selected from pyrrolidinyl (e.g.,

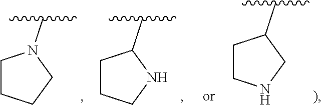 [SMILES: *N1CCCC1 ; *C1CCCN1 ; *C1CCNC1]

piperazinyl (e.g.,

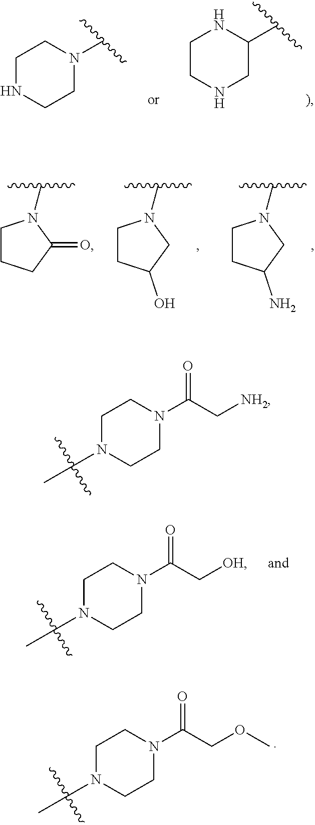

Provided is the compound, and the stereoisomer, the optical isomer, the pharmaceutically acceptable salt, the prodrug, and the solvate thereof described herein, which are preferably selected from the following compounds:

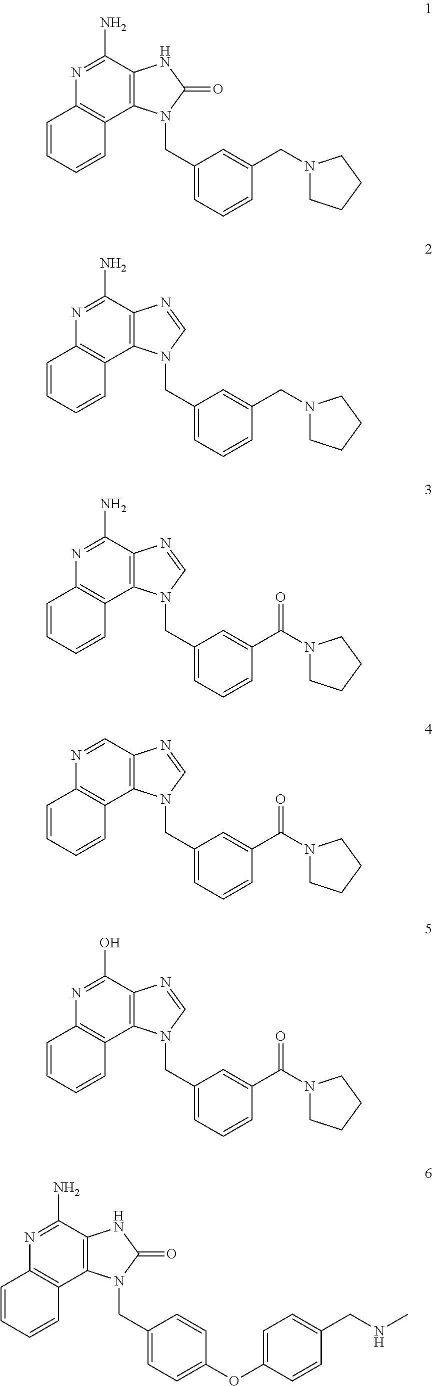 [SMILES: Nc1nc2ccccc2c2c1[nH]c(=O)n2Cc1cccc(CN2CCCC2)c1 ; Nc1nc2ccccc2c2c1ncn2Cc1cccc(CN2CCCC2)c1 ; Nc1nc2ccccc2c2c1ncn2Cc1cccc(C(=O)N2CCCC2)c1 ; O=C(c1cccc(Cn2cnc3cnc4ccccc4c32)c1)N1CCCC1 ; O=C(c1cccc(Cn2cnc3c(O)nc4ccccc4c32)c1)N1CCCC1 ; CNCc1ccc(Oc2ccc(Cn3c(=O)[nH]c4c(N)nc5ccccc5c43)cc2)cc1]

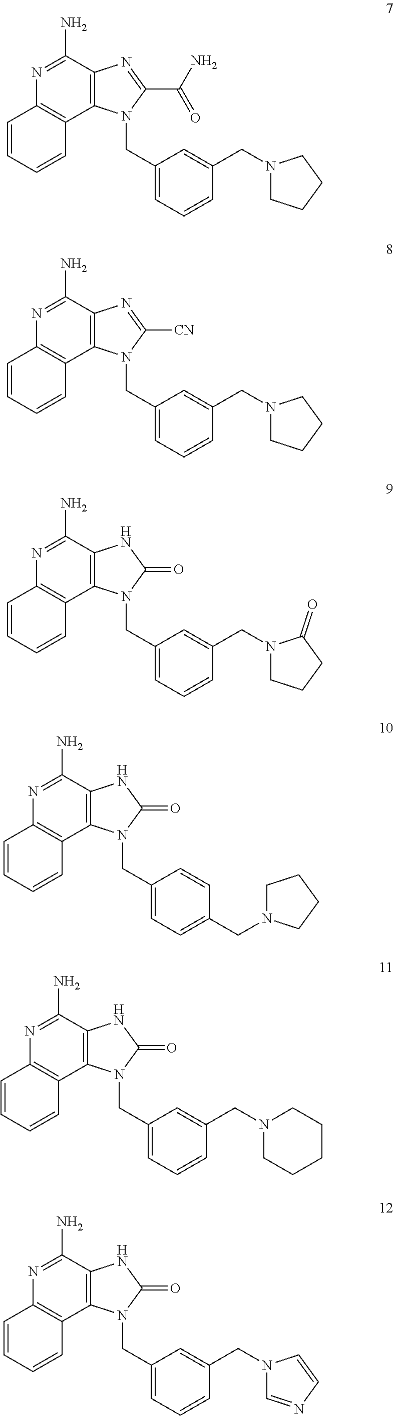 [SMILES: NC(=O)c1nc2c(N)nc3ccccc3c2n1Cc1cccc(CN2CCCC2)c1 ; N#Cc1nc2c(N)nc3ccccc3c2n1Cc1cccc(CN2CCCC2)c1 ; Nc1nc2ccccc2c2c1[nH]c(=O)n2Cc1cccc(CN2CCCC2=O)c1 ; Nc1nc2ccccc2c2c1[nH]c(=O)n2Cc1ccc(CN2CCCC2)cc1 ; Nc1nc2ccccc2c2c1[nH]c(=O)n2Cc1cccc(CN2CCCCC2)c1 ; Nc1nc2ccccc2c2c1[nH]c(=O)n2Cc1cccc(Cn2ccnc2)c1]

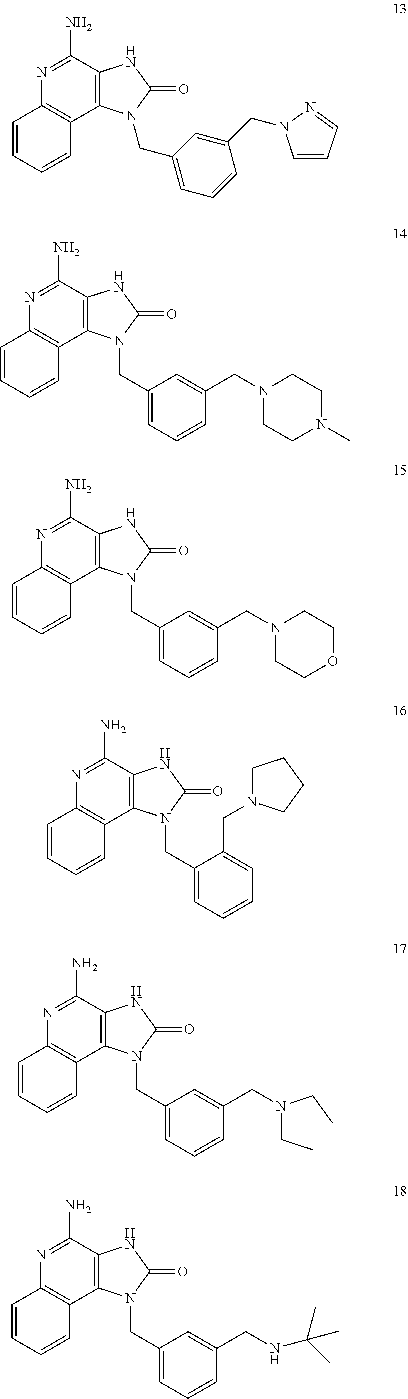 [SMILES: Nc1nc2ccccc2c2c1[nH]c(=O)n2Cc1cccc(Cn2cccn2)c1 ; CN1CCN(Cc2cccc(Cn3c(=O)[nH]c4c(N)nc5ccccc5c43)c2)CC1 ; Nc1nc2ccccc2c2c1[nH]c(=O)n2Cc1cccc(CN2CCOCC2)c1 ; Nc1nc2ccccc2c2c1[nH]c(=O)n2Cc1ccccc1CN1CCCC1 ; CCN(CC)Cc1cccc(Cn2c(=O)[nH]c3c(N)nc4ccccc4c32)c1 ; CC(C)(C)NCc1cccc(Cn2c(=O)[nH]c3c(N)nc4ccccc4c32)c1]

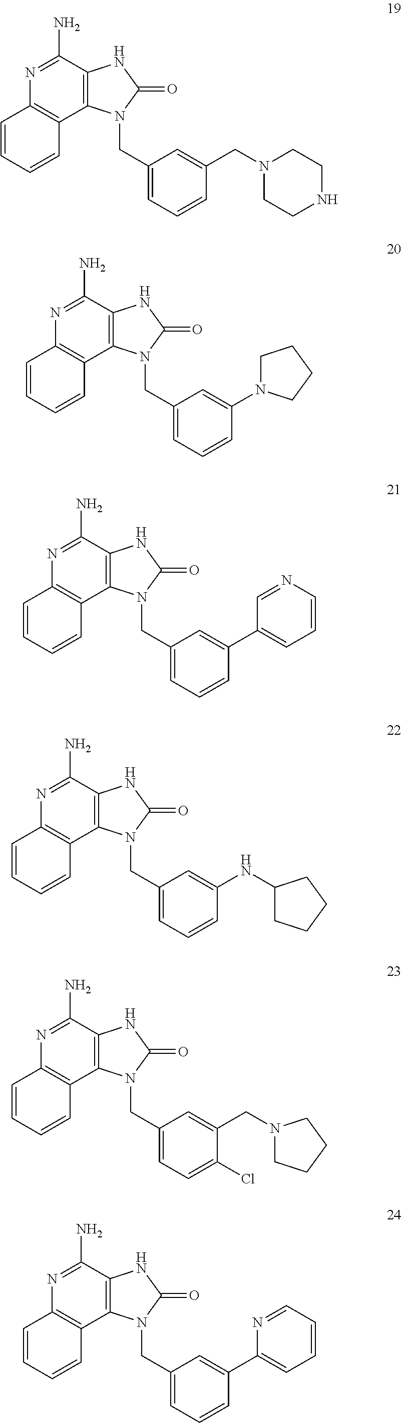 [SMILES: Nc1nc2ccccc2c2c1[nH]c(=O)n2Cc1cccc(CN2CCNCC2)c1 ; Nc1nc2ccccc2c2c1[nH]c(=O)n2Cc1cccc(N2CCCC2)c1 ; Nc1nc2ccccc2c2c1[nH]c(=O)n2Cc1cccc(-c2cccnc2)c1 ; Nc1nc2ccccc2c2c1[nH]c(=O)n2Cc1cccc(NC2CCCC2)c1 ; Nc1nc2ccccc2c2c1[nH]c(=O)n2Cc1ccc(Cl)c(CN2CCCC2)c1 ; Nc1nc2ccccc2c2c1[nH]c(=O)n2Cc1cccc(-c2ccccn2)c1]

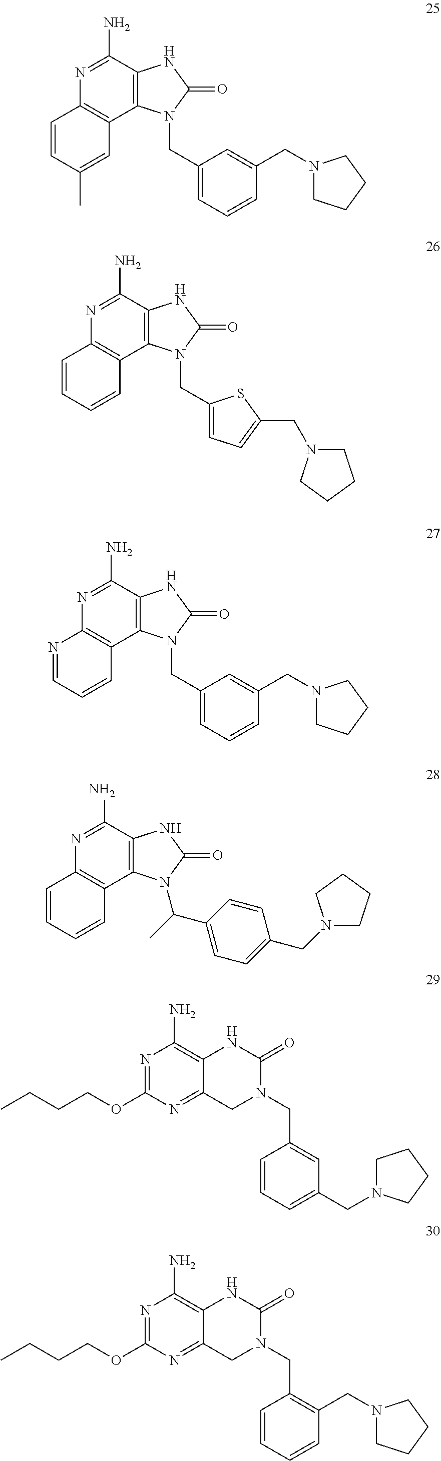 [SMILES: Cc1ccc2nc(N)c3[nH]c(=O)n(Cc4cccc(CN5CCCC5)c4)c3c2c1 ; Nc1nc2ccccc2c2c1[nH]c(=O)n2Cc1ccc(CN2CCCC2)s1 ; Nc1nc2ncccc2c2c1[nH]c(=O)n2Cc1cccc(CN2CCCC2)c1 ; CC(c1ccc(CN2CCCC2)cc1)n1c(=O)[nH]c2c(N)nc3ccccc3c21 ; CCCCOc1nc(N)c2c(n1)CN(Cc1cccc(CN3CCCC3)c1)C(=O)N2 ; CCCCOc1nc(N)c2c(n1)CN(Cc1ccccc1CN1CCCC1)C(=O)N2]

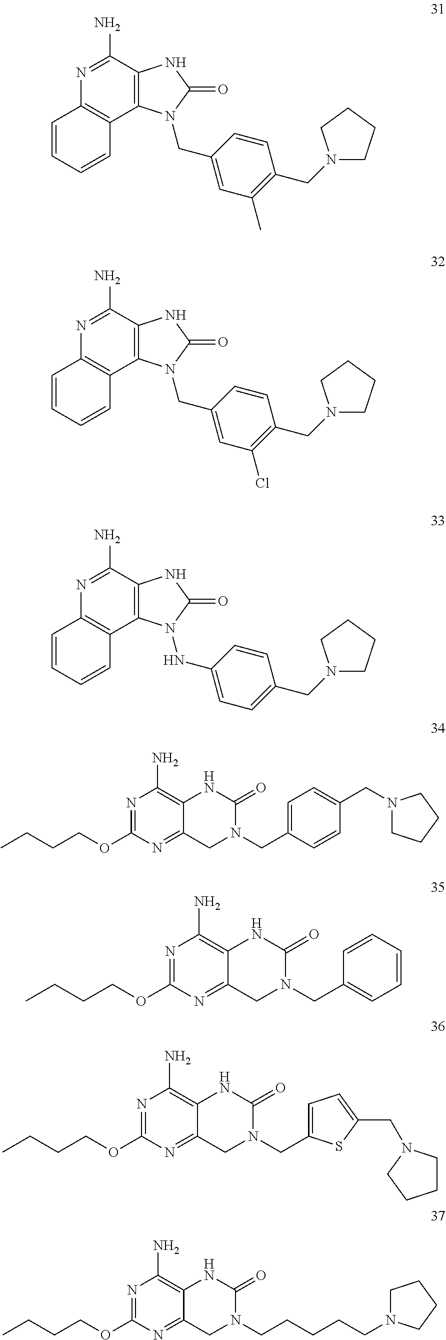

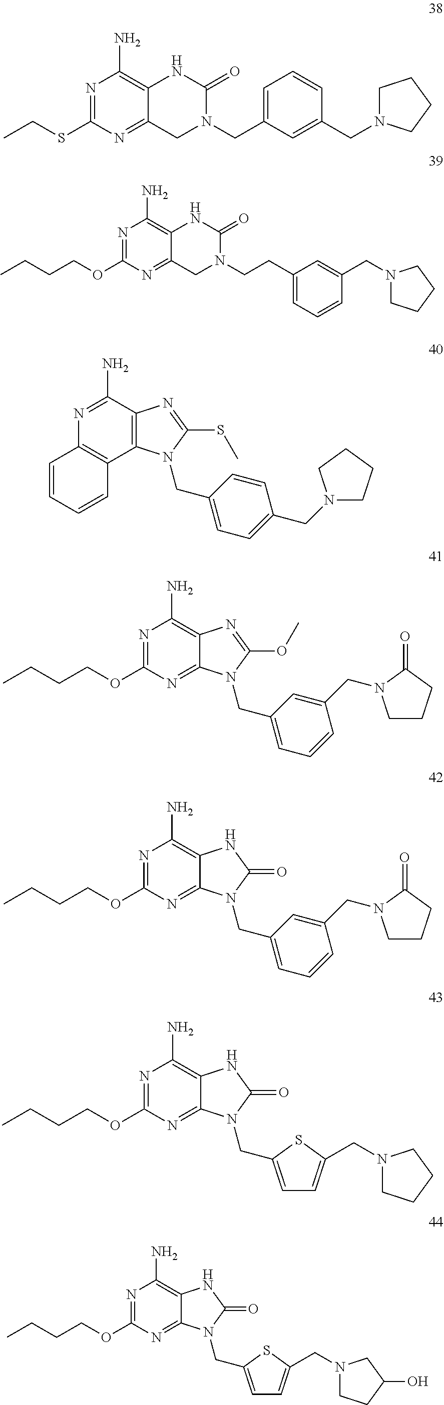

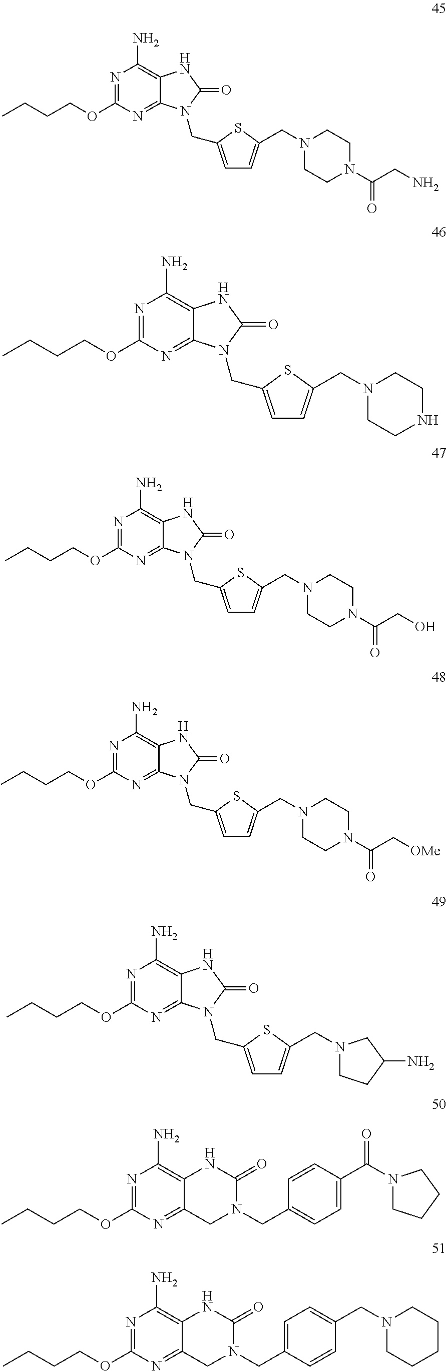

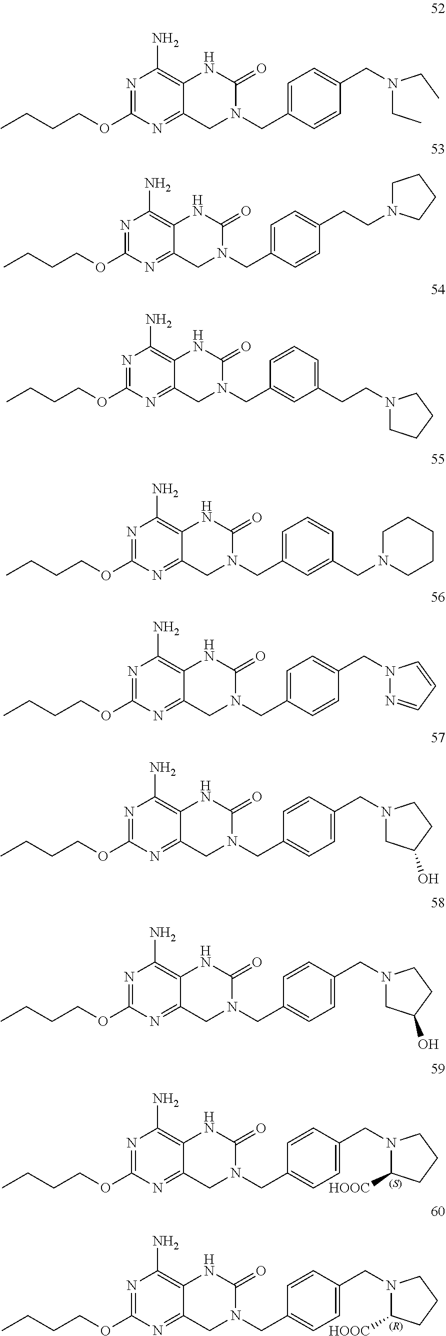

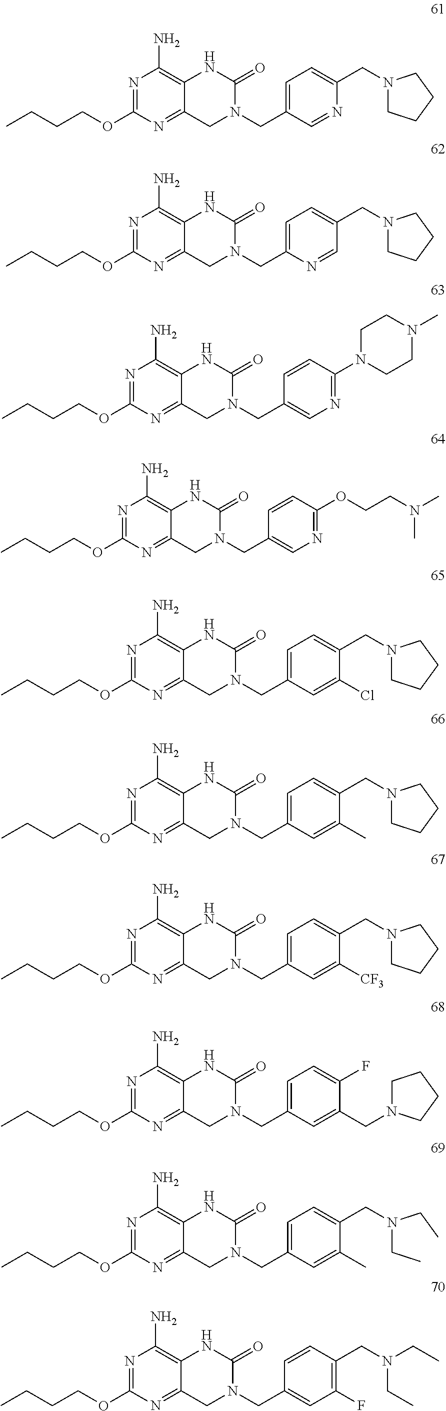

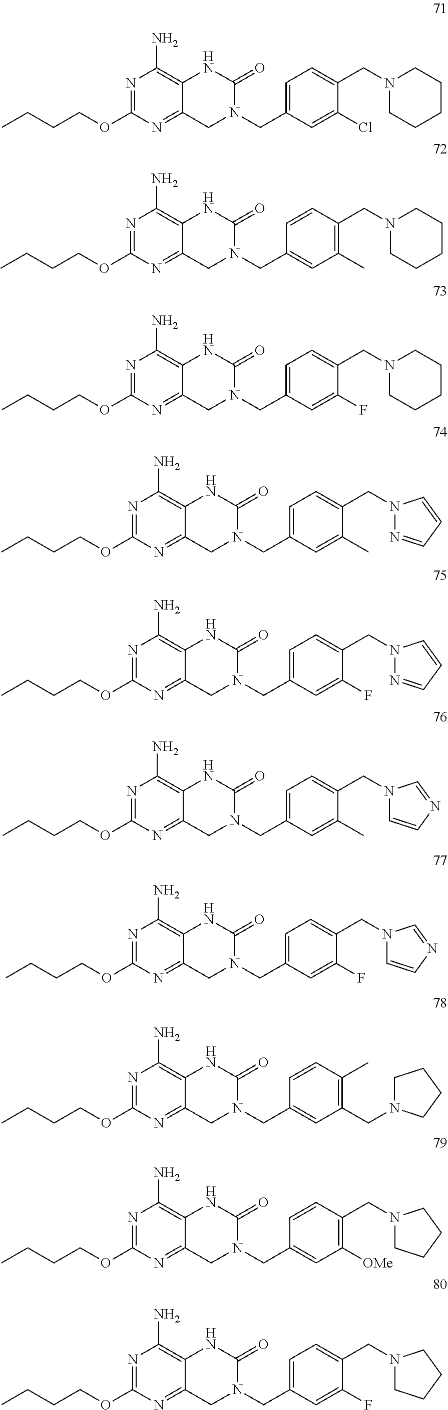

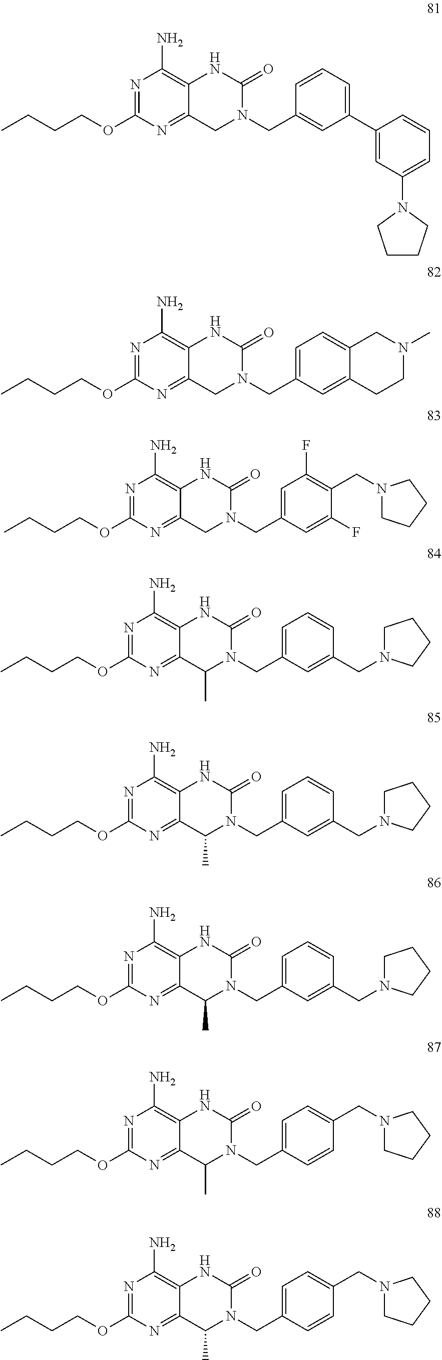

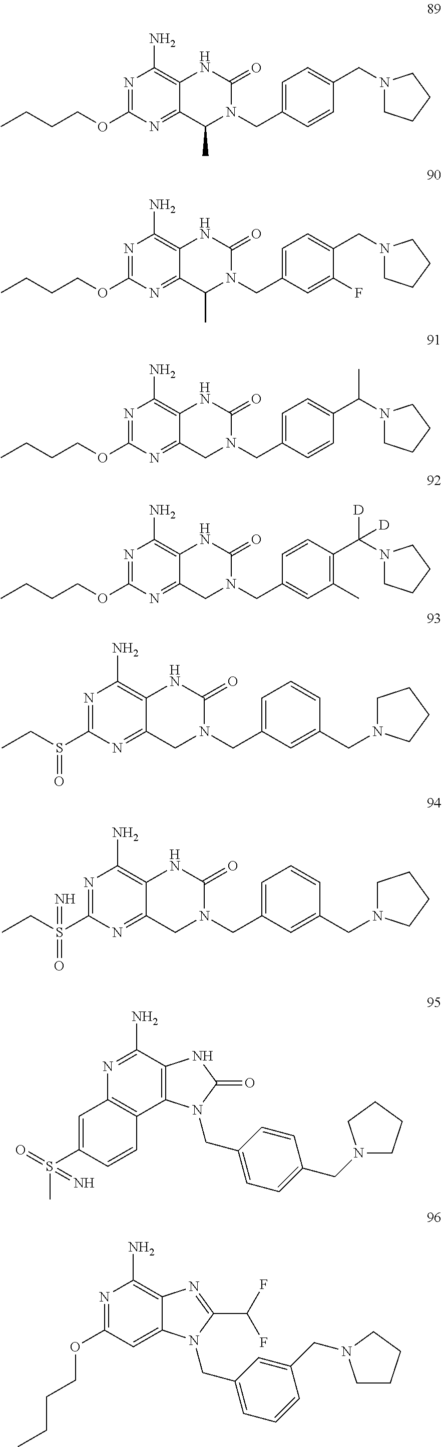

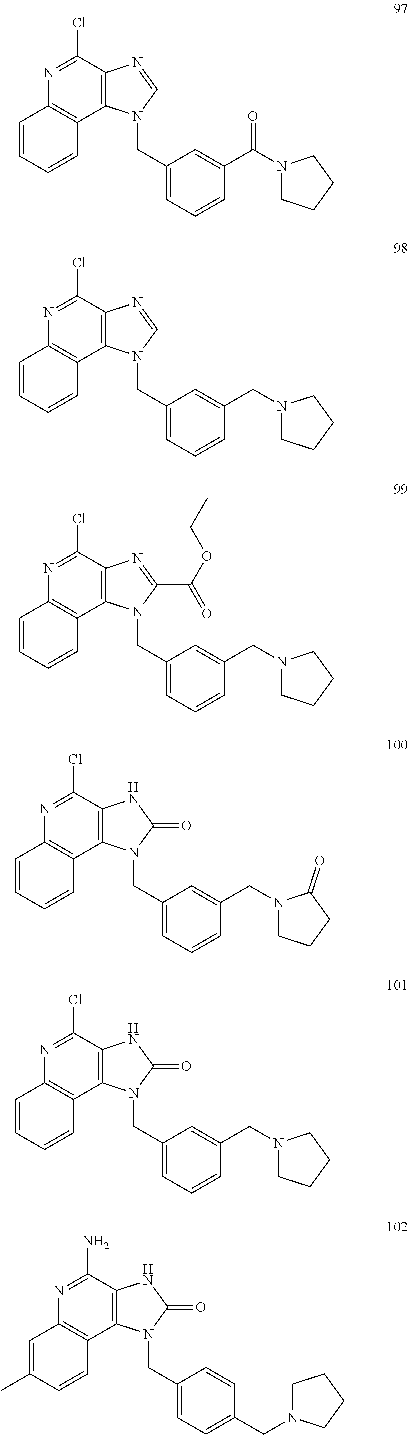

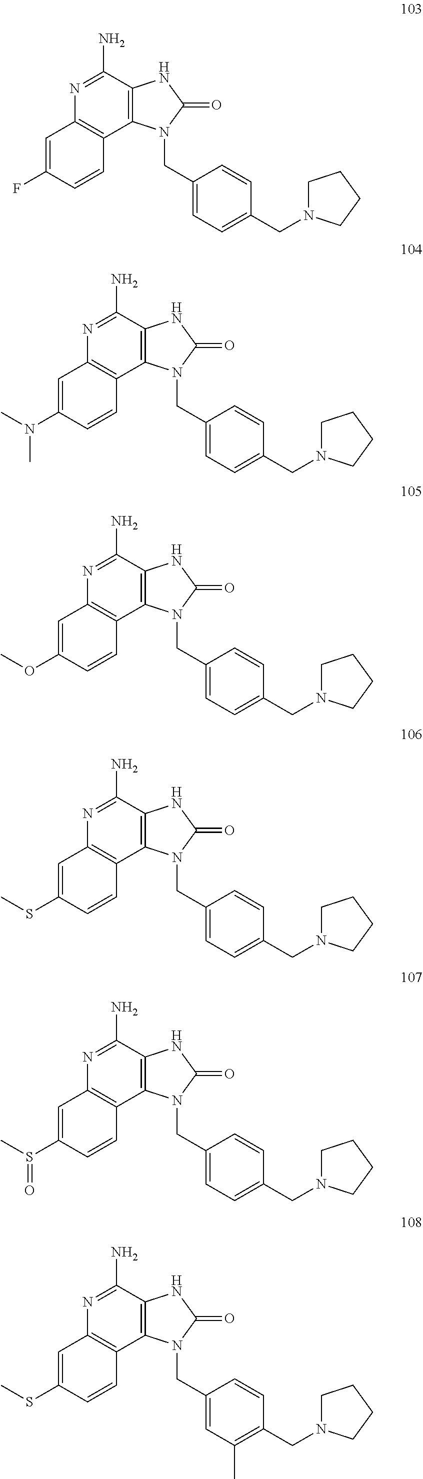 [SMILES: Nc1nc2cc(F)ccc2c2c1[nH]c(=O)n2Cc1ccc(CN2CCCC2)cc1 ; CN(C)c1ccc2c(c1)nc(N)c1[nH]c(=O)n(Cc3ccc(CN4CCCC4)cc3)c12 ; COc1ccc2c(c1)nc(N)c1[nH]c(=O)n(Cc3ccc(CN4CCCC4)cc3)c12 ; CSc1ccc2c(c1)nc(N)c1[nH]c(=O)n(Cc3ccc(CN4CCCC4)cc3)c12 ; CS(=O)c1ccc2c(c1)nc(N)c1[nH]c(=O)n(Cc3ccc(CN4CCCC4)cc3)c12 ; CSc1ccc2c(c1)nc(N)c1[nH]c(=O)n(Cc3ccc(CN4CCCC4)c(C)c3)c12]

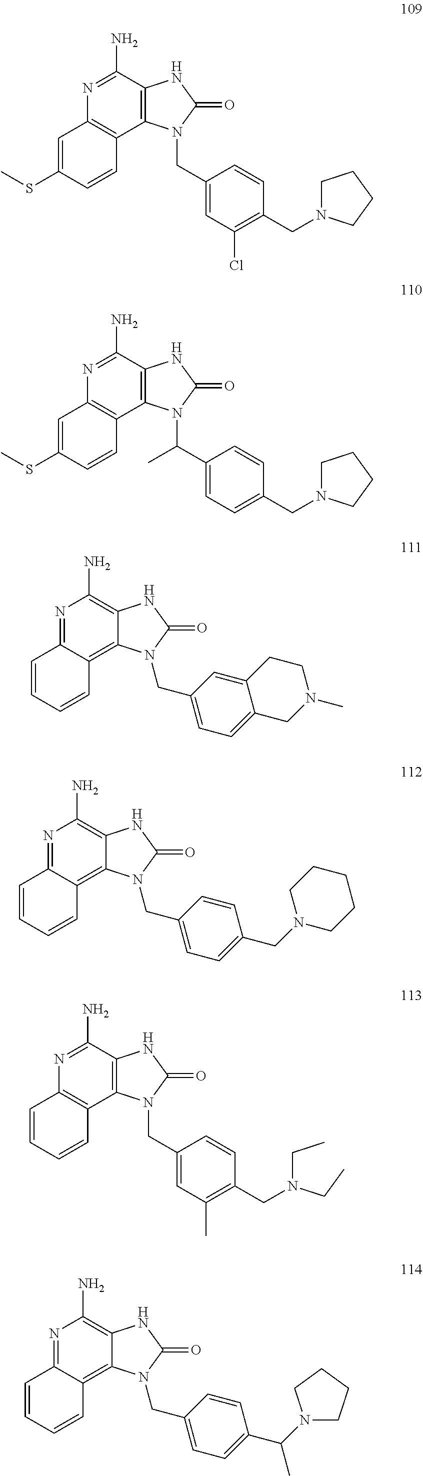 [SMILES: CSc1ccc2c(c1)nc(N)c1[nH]c(=O)n(Cc3ccc(CN4CCCC4)c(Cl)c3)c12 ; CSc1ccc2c(c1)nc(N)c1[nH]c(=O)n(C(C)c3ccc(CN4CCCC4)cc3)c12 ; CN1CCc2cc(Cn3c(=O)[nH]c4c(N)nc5ccccc5c43)ccc2C1 ; Nc1nc2ccccc2c2c1[nH]c(=O)n2Cc1ccc(CN2CCCCC2)cc1 ; CCN(CC)Cc1ccc(Cn2c(=O)[nH]c3c(N)nc4ccccc4c32)cc1C ; CC(c1ccc(Cn2c(=O)[nH]c3c(N)nc4ccccc4c32)cc1)N1CCCC1]

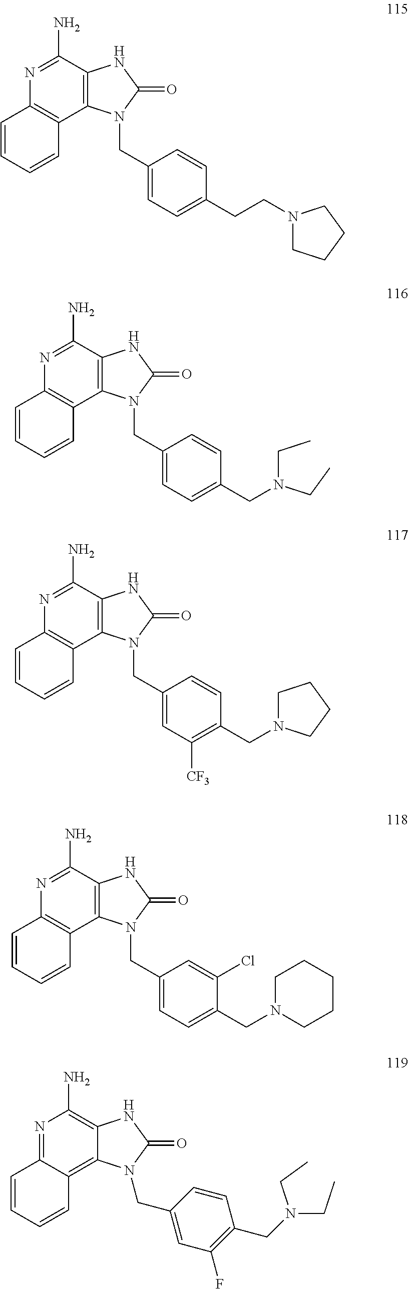 [SMILES: Nc1nc2ccccc2c2c1[nH]c(=O)n2Cc1ccc(CCN2CCCC2)cc1 ; CCN(CC)Cc1ccc(Cn2c(=O)[nH]c3c(N)nc4ccccc4c32)cc1 ; Nc1nc2ccccc2c2c1[nH]c(=O)n2Cc1ccc(CN2CCCC2)c(C(F)(F)F)c1 ; Nc1nc2ccccc2c2c1[nH]c(=O)n2Cc1ccc(CN2CCCCC2)c(Cl)c1 ; CCN(CC)Cc1ccc(Cn2c(=O)[nH]c3c(N)nc4ccccc4c32)cc1F]

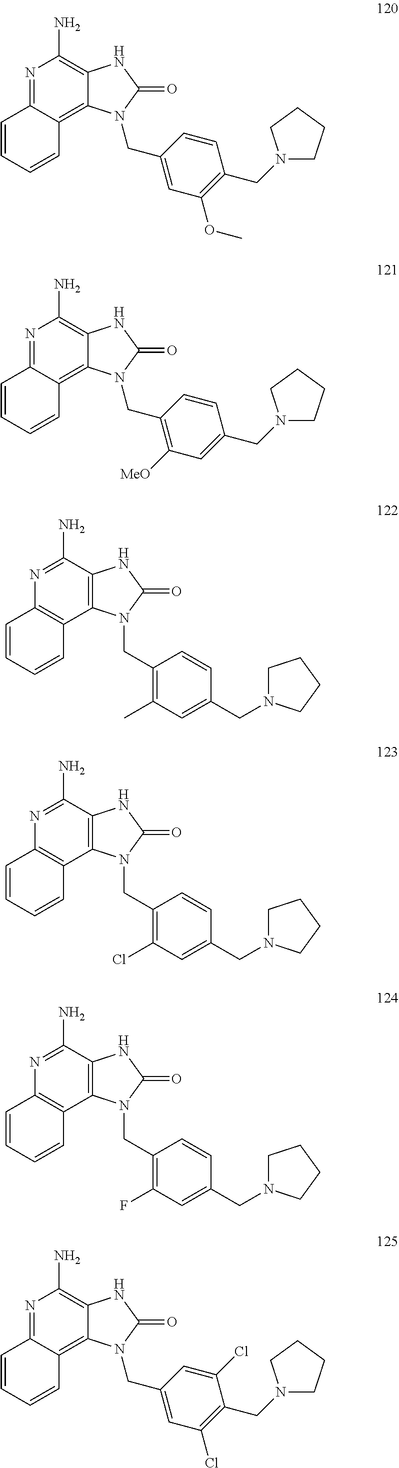 [SMILES: COc1cc(Cn2c(=O)[nH]c3c(N)nc4ccccc4c32)ccc1CN1CCCC1 ; COc1cc(CN2CCCC2)ccc1Cn1c(=O)[nH]c2c(N)nc3ccccc3c21 ; Cc1cc(CN2CCCC2)ccc1Cn1c(=O)[nH]c2c(N)nc3ccccc3c21 ; Nc1nc2ccccc2c2c1[nH]c(=O)n2Cc1ccc(CN2CCCC2)cc1Cl ; Nc1nc2ccccc2c2c1[nH]c(=O)n2Cc1ccc(CN2CCCC2)cc1F ; Nc1nc2ccccc2c2c1[nH]c(=O)n2Cc1cc(Cl)c(CN2CCCC2)c(Cl)c1]

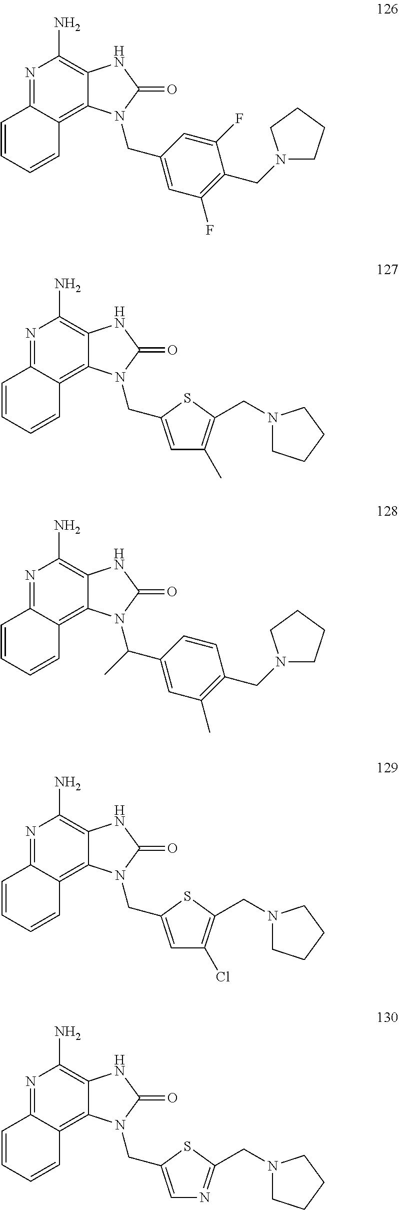 [SMILES: Nc1nc2ccccc2c2c1[nH]c(=O)n2Cc1cc(F)c(CN2CCCC2)c(F)c1 ; Cc1cc(Cn2c(=O)[nH]c3c(N)nc4ccccc4c32)sc1CN1CCCC1 ; Cc1cc(C(C)n2c(=O)[nH]c3c(N)nc4ccccc4c32)ccc1CN1CCCC1 ; Nc1nc2ccccc2c2c1[nH]c(=O)n2Cc1cc(Cl)c(CN2CCCC2)s1 ; Nc1nc2ccccc2c2c1[nH]c(=O)n2Cc1cnc(CN2CCCC2)s1]

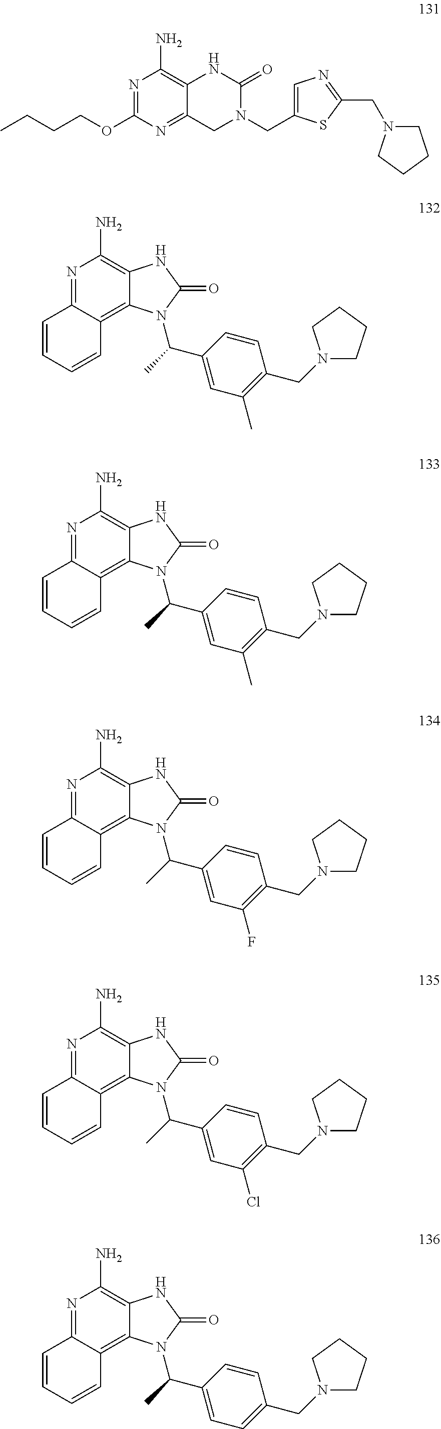 [SMILES: CCCCOc1nc(N)c2c(n1)CN(Cc1cnc(CN3CCCC3)s1)C(=O)N2 ; Cc1cc([C@H](C)n2c(=O)[nH]c3c(N)nc4ccccc4c32)ccc1CN1CCCC1 ; Cc1cc([C@@H](C)n2c(=O)[nH]c3c(N)nc4ccccc4c32)ccc1CN1CCCC1 ; CC(c1ccc(CN2CCCC2)c(F)c1)n1c(=O)[nH]c2c(N)nc3ccccc3c21 ; CC(c1ccc(CN2CCCC2)c(Cl)c1)n1c(=O)[nH]c2c(N)nc3ccccc3c21 ; C[C@H](c1ccc(CN2CCCC2)cc1)n1c(=O)[nH]c2c(N)nc3ccccc3c21]

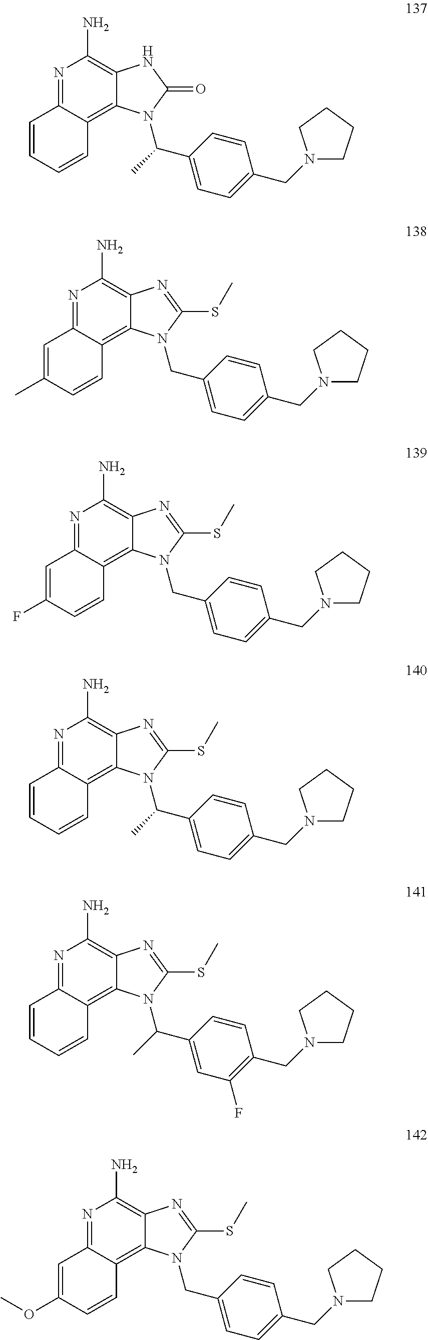 [SMILES: C[C@@H](c1ccc(CN2CCCC2)cc1)n1c(=O)[nH]c2c(N)nc3ccccc3c21 ; CSc1nc2c(N)nc3cc(C)ccc3c2n1Cc1ccc(CN2CCCC2)cc1 ; CSc1nc2c(N)nc3cc(F)ccc3c2n1Cc1ccc(CN2CCCC2)cc1 ; CSc1nc2c(N)nc3ccccc3c2n1[C@@H](C)c1ccc(CN2CCCC2)cc1 ; CSc1nc2c(N)nc3ccccc3c2n1C(C)c1ccc(CN2CCCC2)c(F)c1 ; COc1ccc2c(c1)nc(N)c1nc(SC)n(Cc3ccc(CN4CCCC4)cc3)c12]

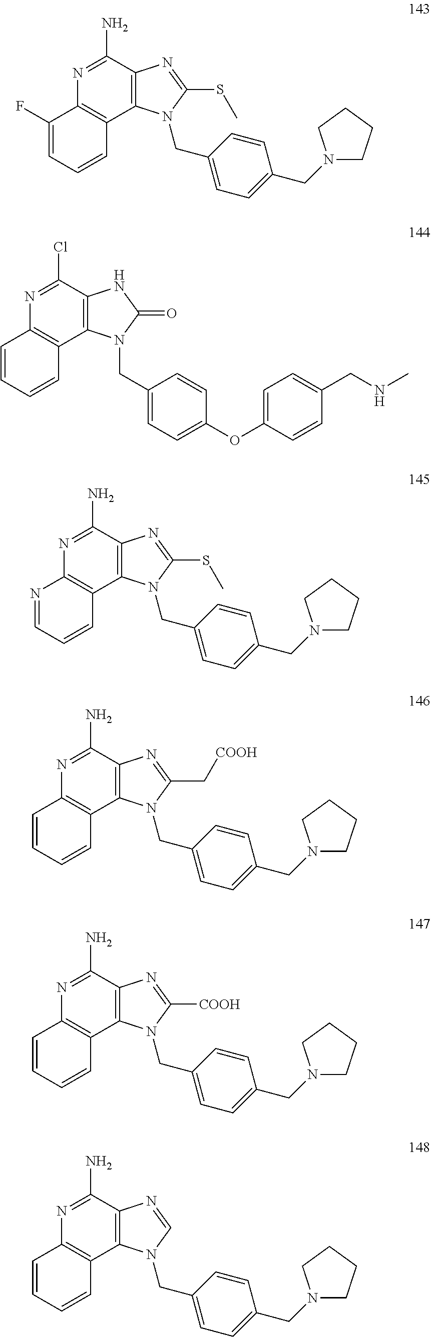 [SMILES: CSc1nc2c(N)nc3c(F)cccc3c2n1Cc1ccc(CN2CCCC2)cc1 ; CNCc1ccc(Oc2ccc(Cn3c(=O)[nH]c4c(Cl)nc5ccccc5c43)cc2)cc1 ; CSc1nc2c(N)nc3ncccc3c2n1Cc1ccc(CN2CCCC2)cc1 ; Nc1nc2ccccc2c2c1nc(CC(=O)O)n2Cc1ccc(CN2CCCC2)cc1 ; Nc1nc2ccccc2c2c1nc(C(=O)O)n2Cc1ccc(CN2CCCC2)cc1 ; Nc1nc2ccccc2c2c1ncn2Cc1ccc(CN2CCCC2)cc1]

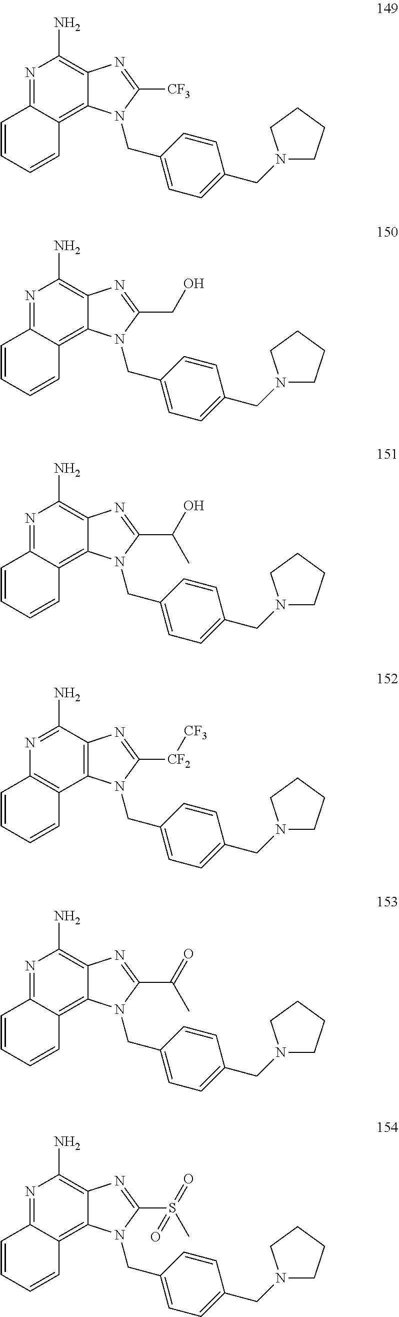 [SMILES: Nc1nc2ccccc2c2c1nc(C(F)(F)F)n2Cc1ccc(CN2CCCC2)cc1 ; Nc1nc2ccccc2c2c1nc(CO)n2Cc1ccc(CN2CCCC2)cc1 ; CC(O)c1nc2c(N)nc3ccccc3c2n1Cc1ccc(CN2CCCC2)cc1 ; Nc1nc2ccccc2c2c1nc(C(F)(F)C(F)(F)F)n2Cc1ccc(CN2CCCC2)cc1 ; CC(=O)c1nc2c(N)nc3ccccc3c2n1Cc1ccc(CN2CCCC2)cc1 ; CS(=O)(=O)c1nc2c(N)nc3ccccc3c2n1Cc1ccc(CN2CCCC2)cc1]

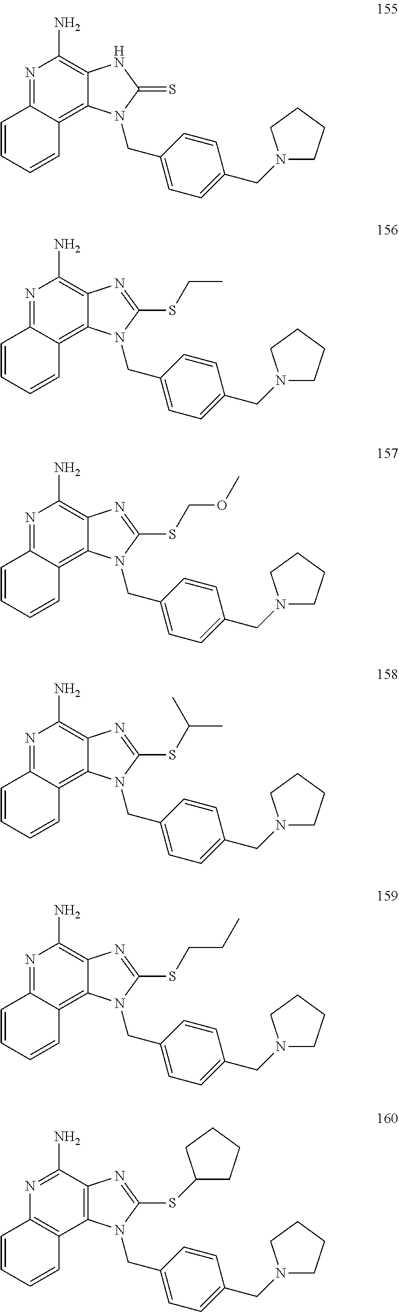

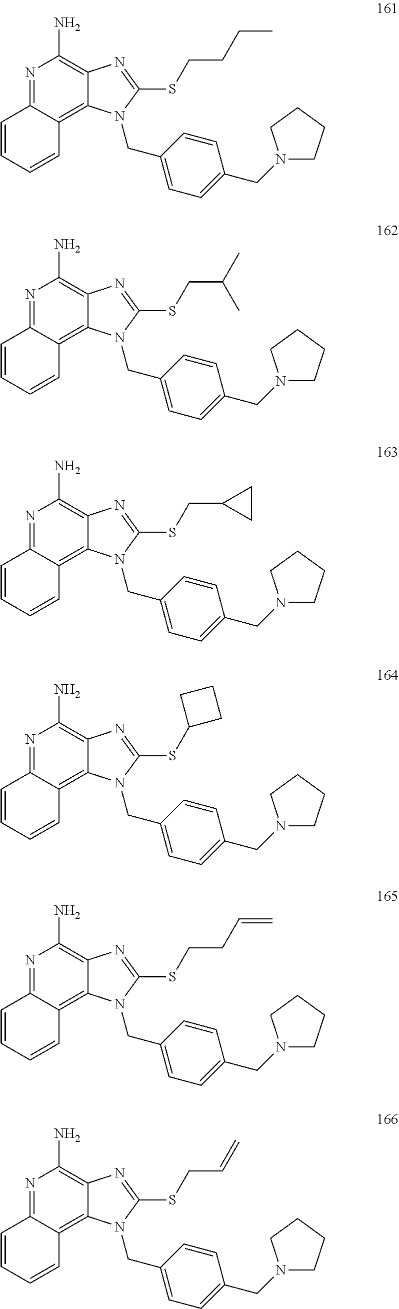

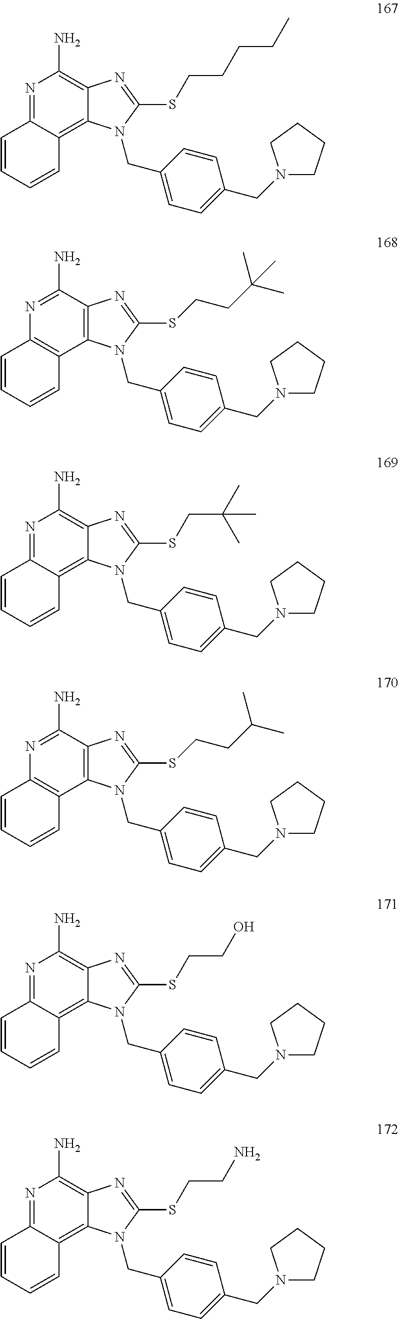 [SMILES: CCCCCSc1nc2c(N)nc3ccccc3c2n1Cc1ccc(CN2CCCC2)cc1 ; CC(C)(C)CCSc1nc2c(N)nc3ccccc3c2n1Cc1ccc(CN2CCCC2)cc1 ; CC(C)(C)CSc1nc2c(N)nc3ccccc3c2n1Cc1ccc(CN2CCCC2)cc1 ; CC(C)CCSc1nc2c(N)nc3ccccc3c2n1Cc1ccc(CN2CCCC2)cc1 ; Nc1nc2ccccc2c2c1nc(SCCO)n2Cc1ccc(CN2CCCC2)cc1 ; NCCSc1nc2c(N)nc3ccccc3c2n1Cc1ccc(CN2CCCC2)cc1]

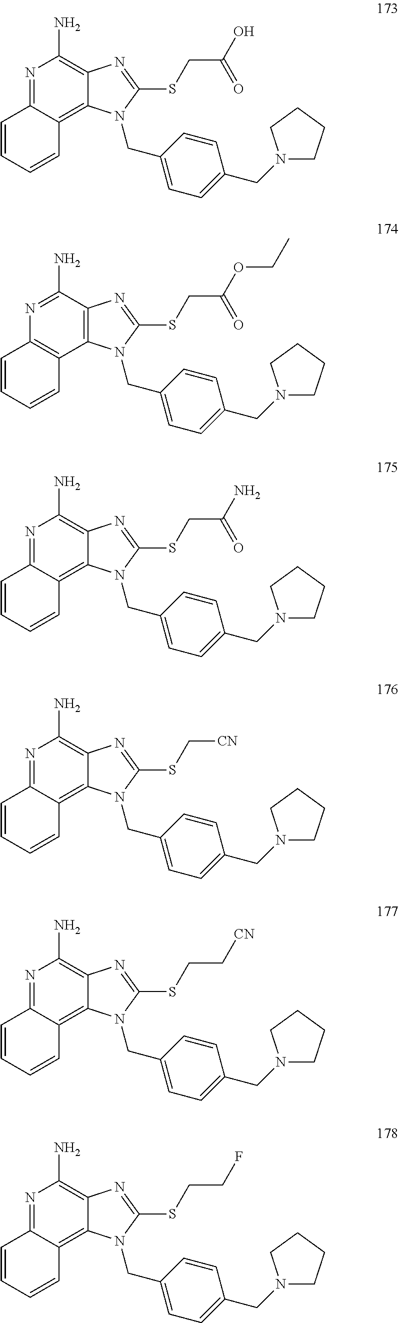 [SMILES: Nc1nc2ccccc2c2c1nc(SCC(=O)O)n2Cc1ccc(CN2CCCC2)cc1 ; CCOC(=O)CSc1nc2c(N)nc3ccccc3c2n1Cc1ccc(CN2CCCC2)cc1 ; NC(=O)CSc1nc2c(N)nc3ccccc3c2n1Cc1ccc(CN2CCCC2)cc1 ; N#CCSc1nc2c(N)nc3ccccc3c2n1Cc1ccc(CN2CCCC2)cc1 ; N#CCCSc1nc2c(N)nc3ccccc3c2n1Cc1ccc(CN2CCCC2)cc1 ; Nc1nc2ccccc2c2c1nc(SCCF)n2Cc1ccc(CN2CCCC2)cc1]

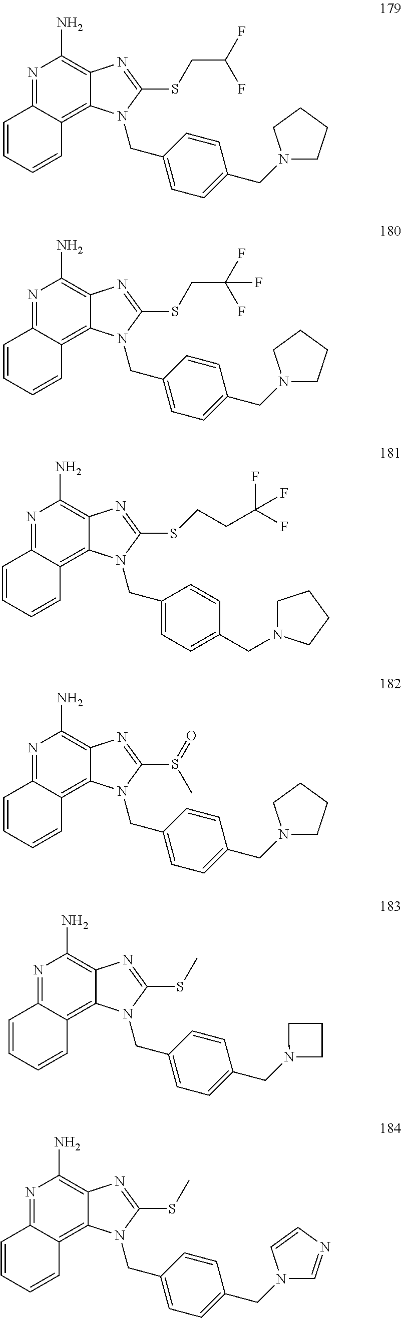 [SMILES: Nc1nc2ccccc2c2c1nc(SCC(F)F)n2Cc1ccc(CN2CCCC2)cc1 ; Nc1nc2ccccc2c2c1nc(SCC(F)(F)F)n2Cc1ccc(CN2CCCC2)cc1 ; Nc1nc2ccccc2c2c1nc(SCCC(F)(F)F)n2Cc1ccc(CN2CCCC2)cc1 ; CS(=O)c1nc2c(N)nc3ccccc3c2n1Cc1ccc(CN2CCCC2)cc1 ; CSc1nc2c(N)nc3ccccc3c2n1Cc1ccc(CN2CCC2)cc1 ; CSc1nc2c(N)nc3ccccc3c2n1Cc1ccc(Cn2ccnc2)cc1]

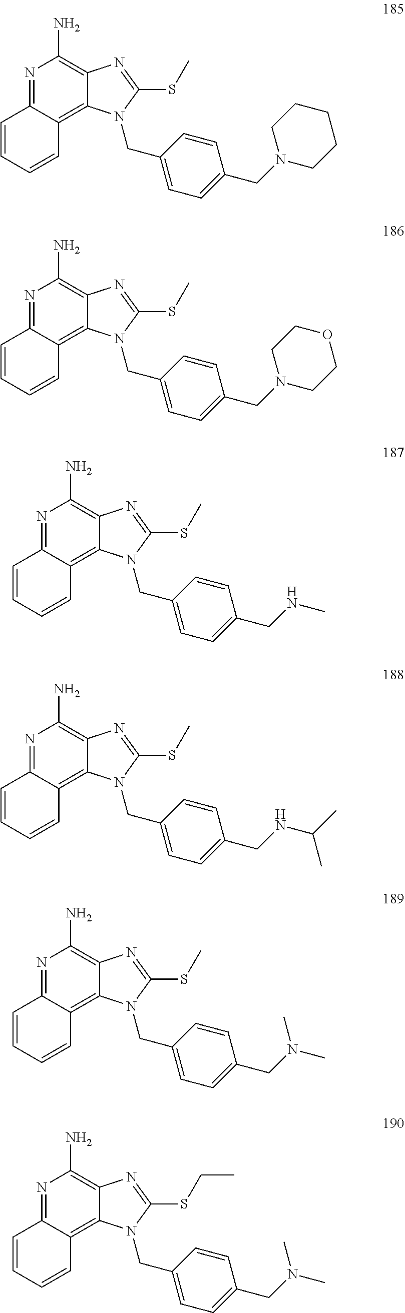 [SMILES: CSc1nc2c(N)nc3ccccc3c2n1Cc1ccc(CN2CCCCC2)cc1 ; CSc1nc2c(N)nc3ccccc3c2n1Cc1ccc(CN2CCOCC2)cc1 ; CNCc1ccc(Cn2c(SC)nc3c(N)nc4ccccc4c32)cc1 ; CSc1nc2c(N)nc3ccccc3c2n1Cc1ccc(CNC(C)C)cc1 ; CSc1nc2c(N)nc3ccccc3c2n1Cc1ccc(CN(C)C)cc1 ; CCSc1nc2c(N)nc3ccccc3c2n1Cc1ccc(CN(C)C)cc1]

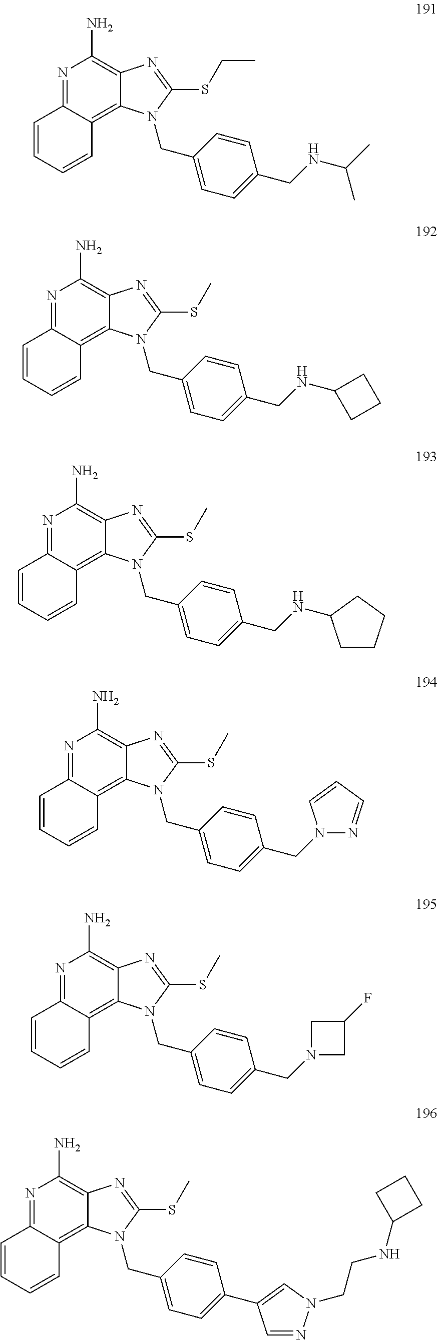 [SMILES: CCSc1nc2c(N)nc3ccccc3c2n1Cc1ccc(CNC(C)C)cc1 ; CSc1nc2c(N)nc3ccccc3c2n1Cc1ccc(CNC2CCC2)cc1 ; CSc1nc2c(N)nc3ccccc3c2n1Cc1ccc(CNC2CCCC2)cc1 ; CSc1nc2c(N)nc3ccccc3c2n1Cc1ccc(Cn2cccn2)cc1 ; CSc1nc2c(N)nc3ccccc3c2n1Cc1ccc(CN2CC(F)C2)cc1 ; CSc1nc2c(N)nc3ccccc3c2n1Cc1ccc(-c2cnn(CCNC3CCC3)c2)cc1]

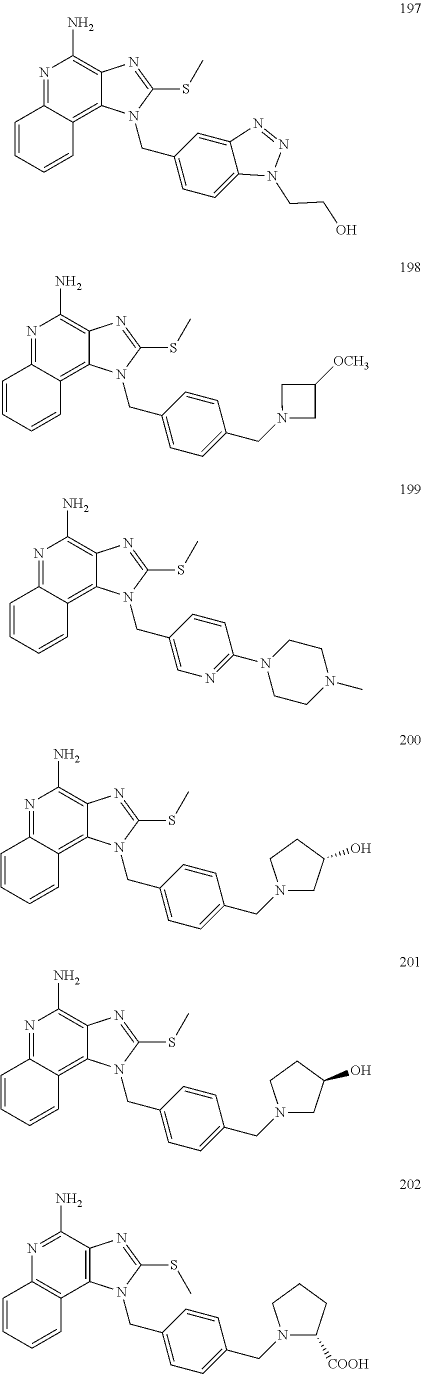 [SMILES: CSc1nc2c(N)nc3ccccc3c2n1Cc1ccc2c(c1)nnn2CCO ; COC1CN(Cc2ccc(Cn3c(SC)nc4c(N)nc5ccccc5c43)cc2)C1 ; CSc1nc2c(N)nc3ccccc3c2n1Cc1ccc(N2CCN(C)CC2)nc1 ; CSc1nc2c(N)nc3ccccc3c2n1Cc1ccc(CN2CC[C@H](O)C2)cc1 ; CSc1nc2c(N)nc3ccccc3c2n1Cc1ccc(CN2CC[C@@H](O)C2)cc1 ; CSc1nc2c(N)nc3ccccc3c2n1Cc1ccc(CN2CCC[C@@H]2C(=O)O)cc1]

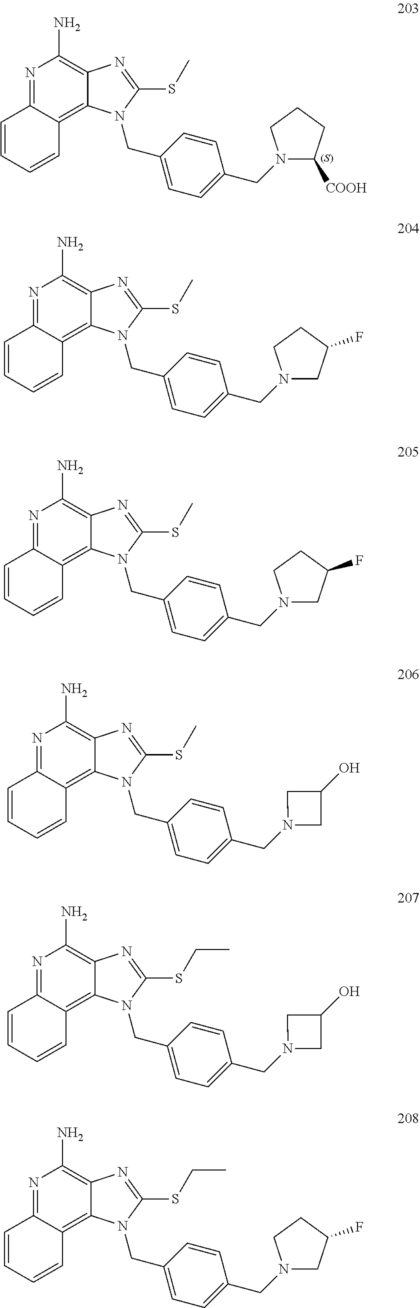

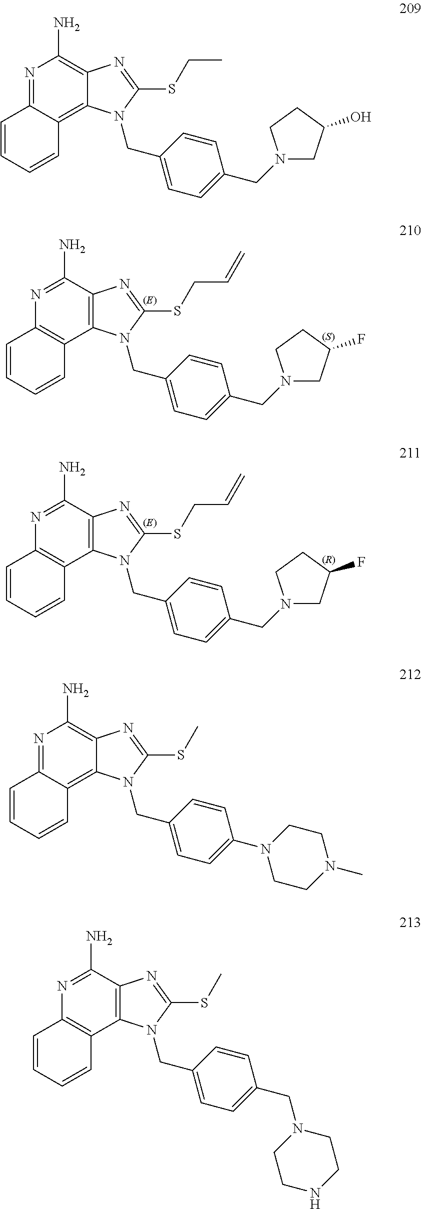

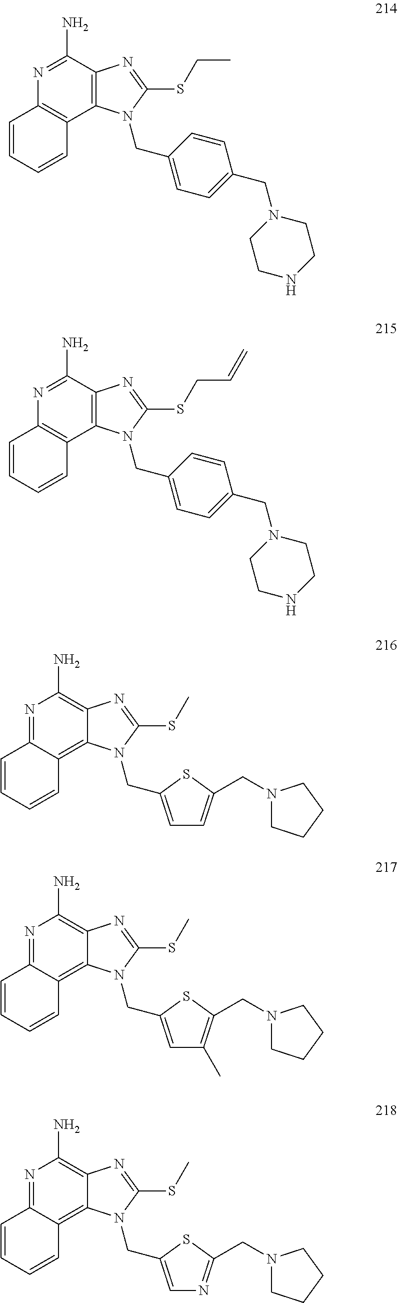 [SMILES: CCSc1nc2c(N)nc3ccccc3c2n1Cc1ccc(CN2CCNCC2)cc1 ; C=CCSc1nc2c(N)nc3ccccc3c2n1Cc1ccc(CN2CCNCC2)cc1 ; CSc1nc2c(N)nc3ccccc3c2n1Cc1ccc(CN2CCCC2)s1 ; CSc1nc2c(N)nc3ccccc3c2n1Cc1cc(C)c(CN2CCCC2)s1 ; CSc1nc2c(N)nc3ccccc3c2n1Cc1cnc(CN2CCCC2)s1]

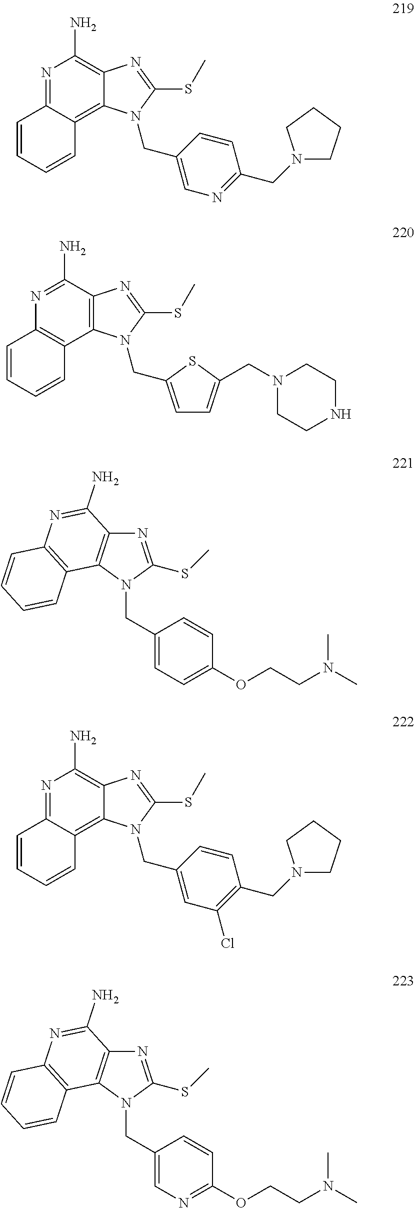 [SMILES: CSc1nc2c(N)nc3ccccc3c2n1Cc1ccc(CN2CCCC2)nc1 ; CSc1nc2c(N)nc3ccccc3c2n1Cc1ccc(CN2CCNCC2)s1 ; CSc1nc2c(N)nc3ccccc3c2n1Cc1ccc(OCCN(C)C)cc1 ; CSc1nc2c(N)nc3ccccc3c2n1Cc1ccc(CN2CCCC2)c(Cl)c1 ; CSc1nc2c(N)nc3ccccc3c2n1Cc1ccc(OCCN(C)C)nc1]

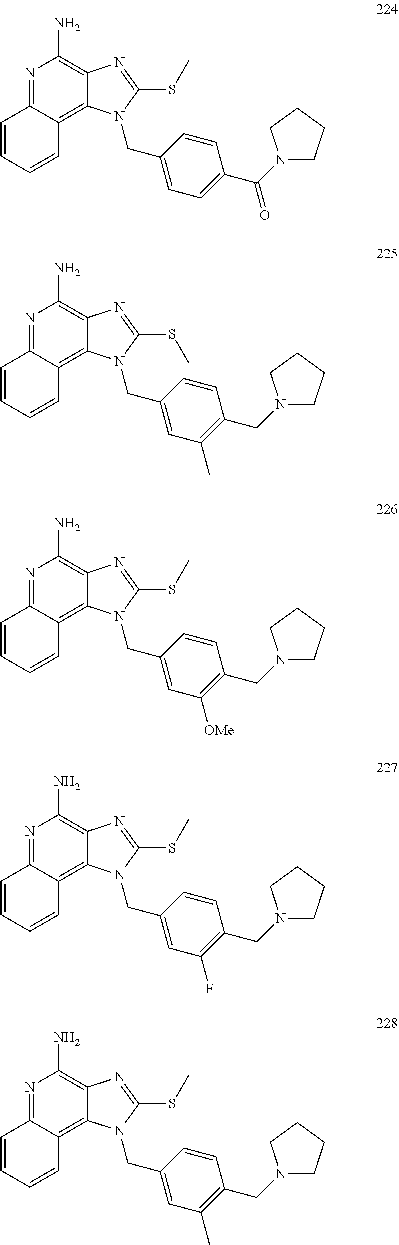 [SMILES: CSc1nc2c(N)nc3ccccc3c2n1Cc1ccc(C(=O)N2CCCC2)cc1 ; CSc1nc2c(N)nc3ccccc3c2n1Cc1ccc(CN2CCCC2)c(C)c1 ; COc1cc(Cn2c(SC)nc3c(N)nc4ccccc4c32)ccc1CN1CCCC1 ; CSc1nc2c(N)nc3ccccc3c2n1Cc1ccc(CN2CCCC2)c(F)c1 ; CSc1nc2c(N)nc3ccccc3c2n1Cc1ccc(CN2CCCC2)c(C)c1]

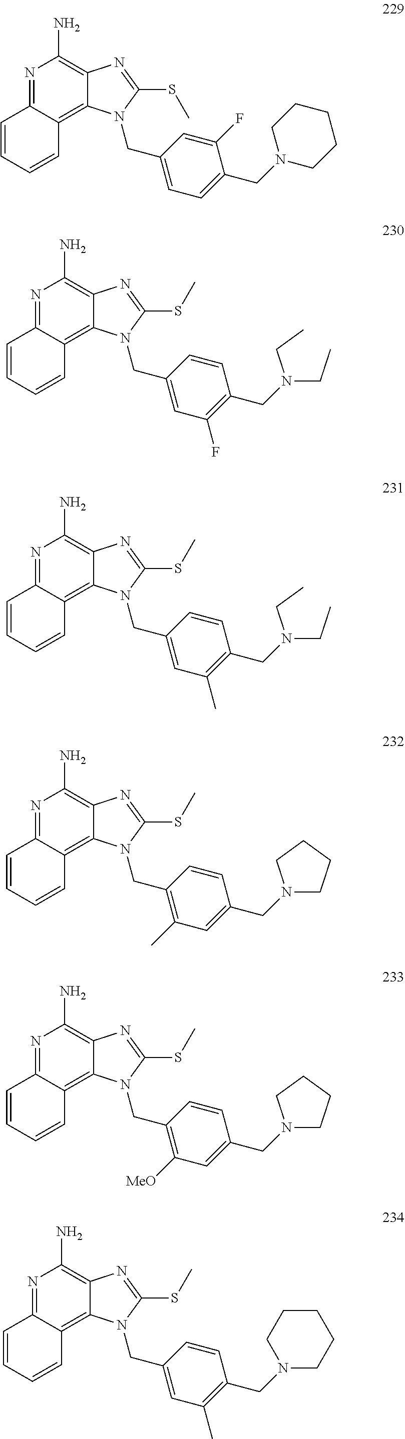 [SMILES: CSc1nc2c(N)nc3ccccc3c2n1Cc1ccc(CN2CCCCC2)c(F)c1 ; CCN(CC)Cc1ccc(Cn2c(SC)nc3c(N)nc4ccccc4c32)cc1F ; CCN(CC)Cc1ccc(Cn2c(SC)nc3c(N)nc4ccccc4c32)cc1C ; CSc1nc2c(N)nc3ccccc3c2n1Cc1ccc(CN2CCCC2)cc1C ; COc1cc(CN2CCCC2)ccc1Cn1c(SC)nc2c(N)nc3ccccc3c21 ; CSc1nc2c(N)nc3ccccc3c2n1Cc1ccc(CN2CCCCC2)c(C)c1]

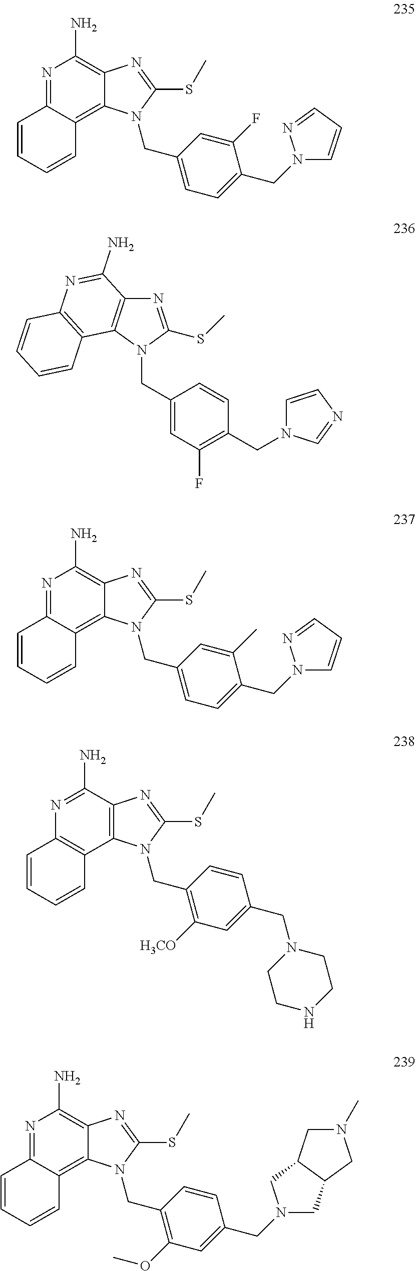 [SMILES: CSc1nc2c(N)nc3ccccc3c2n1Cc1ccc(Cn2cccn2)c(F)c1 ; CSc1nc2c(N)nc3ccccc3c2n1Cc1ccc(Cn2ccnc2)c(F)c1 ; CSc1nc2c(N)nc3ccccc3c2n1Cc1ccc(Cn2cccn2)c(C)c1 ; COc1cc(CN2CCNCC2)ccc1Cn1c(SC)nc2c(N)nc3ccccc3c21 ; COc1cc(CN2C[C@H]3CN(C)C[C@H]3C2)ccc1Cn1c(SC)nc2c(N)nc3ccccc3c21]

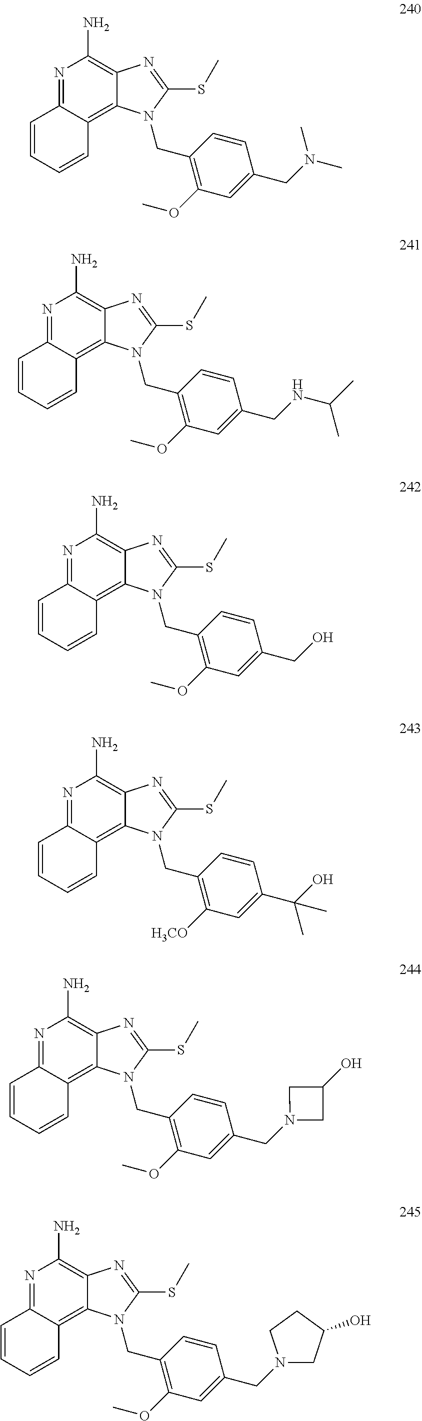 [SMILES: COc1cc(CN(C)C)ccc1Cn1c(SC)nc2c(N)nc3ccccc3c21 ; COc1cc(CNC(C)C)ccc1Cn1c(SC)nc2c(N)nc3ccccc3c21 ; COc1cc(CO)ccc1Cn1c(SC)nc2c(N)nc3ccccc3c21 ; COc1cc(C(C)(C)O)ccc1Cn1c(SC)nc2c(N)nc3ccccc3c21 ; COc1cc(CN2CC(O)C2)ccc1Cn1c(SC)nc2c(N)nc3ccccc3c21 ; COc1cc(CN2CC[C@H](O)C2)ccc1Cn1c(SC)nc2c(N)nc3ccccc3c21]

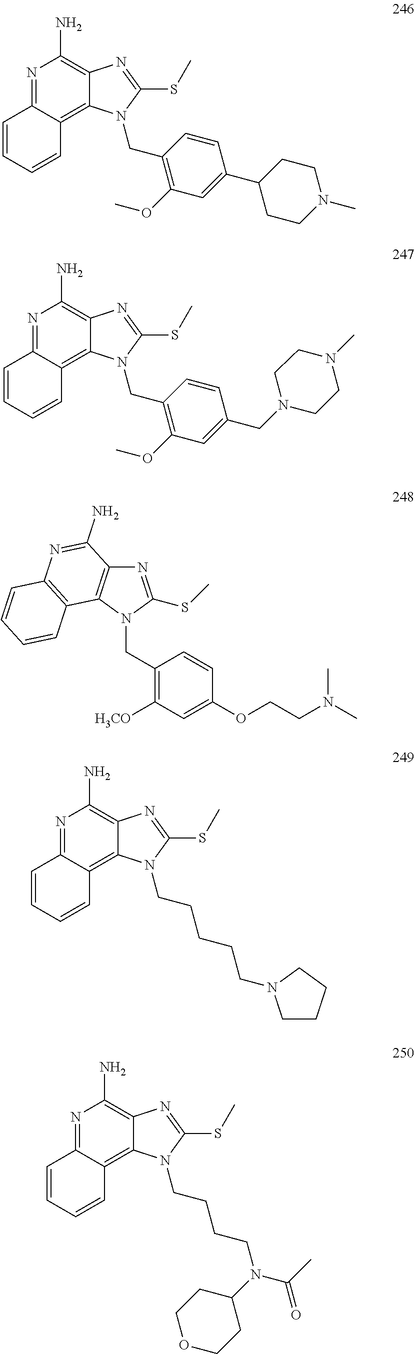 [SMILES: COc1cc(C2CCN(C)CC2)ccc1Cn1c(SC)nc2c(N)nc3ccccc3c21 ; COc1cc(CN2CCN(C)CC2)ccc1Cn1c(SC)nc2c(N)nc3ccccc3c21 ; COc1cc(OCCN(C)C)ccc1Cn1c(SC)nc2c(N)nc3ccccc3c21 ; CSc1nc2c(N)nc3ccccc3c2n1CCCCCN1CCCC1 ; CSc1nc2c(N)nc3ccccc3c2n1CCCCN(C(C)=O)C1CCOCC1]

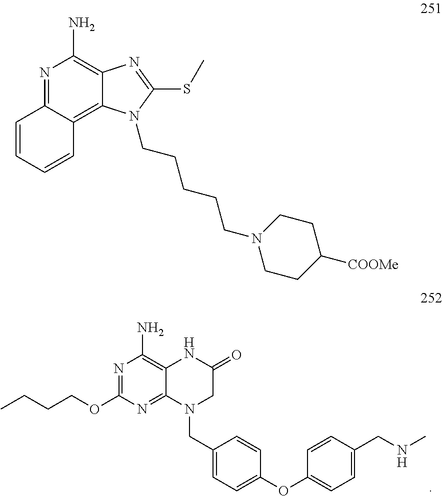 [SMILES: COC(=O)C1CCN(CCCCCn2c(SC)nc3c(N)nc4ccccc4c32)CC1 ; CCCCOc1nc(N)c2c(n1)N(Cc1ccc(Oc3ccc(CNC)cc3)cc1)CC(=O)N2]

The present disclosure further provides a pharmaceutical composition comprising the compound, and the stereoisomer, the optical isomer, the pharmaceutically acceptable salt, the prodrug, and the solvate thereof of the present disclosure.

The present disclosure further provides a pharmaceutical composition comprising the compound, and the stereoisomer, the optical isomer, the pharmaceutically acceptable salt, the prodrug, and the solvate thereof of the present disclosure, and a pharmaceutically acceptable excipient.

It is also an object of the present disclosure to provide the compound, and the stereoisomer, the optical isomer, the pharmaceutically acceptable salt, the prodrug, and the solvate thereof of the present disclosure, or the pharmaceutical composition of the present disclosure, for use in the prevention and/or treatment of diseases at least partially mediated by a TLR7 agonist, preferably for use in the prevention and/or treatment of diseases mediated by a TLR7 agonist, and more preferably for use in the treatment of diseases mediated by a TLR7 agonist.

It is also an object of the present disclosure to provide use of the compound, and the stereoisomer, the optical isomer, the pharmaceutically acceptable salt, the prodrug, and the solvate thereof of the present disclosure, or the pharmaceutical composition of the present disclosure, for manufacturing a medicament for the prevention and/or treatment of diseases at least partially mediated by a TLR7 agonist, preferably for manufacturing a medicament for the prevention and/or treatment of diseases mediated by a TLR7 agonist, and more preferably for manufacturing a medicament for the treatment of diseases mediated by a TLR7 agonist.

Further, the diseases at least partially mediated by the TLR7 agonist (preferably the diseases mediated by the TLR7 agonist) described herein are cancers or virus-infected diseases.

In certain contexts in the art, the cancers may also be referred to as tumors.

Provided is the use of the compound, and the stereoisomer, the optical isomer, the pharmaceutically acceptable salt, the prodrug, and the solvate thereof of the present disclosure, or the pharmaceutical composition of the present disclosure for manufacturing a medicament for the prevention and/or treatment of diseases at least partially mediated by a TLR7 agonist, which may be administered in combination with an additional medicament for the prevention and/or treatment of virus-infected diseases.

Provided is the use of the compound, and the stereoisomer, the optical isomer, the pharmaceutically acceptable salt, the prodrug, and the solvate thereof of the present disclosure, or the pharmaceutical composition of the present disclosure for manufacturing a medicament for the prevention and/or treatment of diseases at least partially mediated by a TLR7 agonist, which may be administered in combination with an additional anti-cancer agent or immune checkpoint inhibitor for the prevention and/or treatment of cancers or tumors.

The compound, and the stereoisomer, the optical isomer, the pharmaceutically acceptable salt, the prodrug, and the solvate thereof of the present disclosure, or the pharmaceutical composition of the present disclosure, can provide an enhanced anti-cancer effect when administered in combination with an additional anti-cancer agent or immune checkpoint inhibitor for the prevention and/or treatment of cancers or tumors.

Further, in some embodiments, the diseases at least partially mediated by the TLR7 agonist (preferably the diseases mediated by the TLR7 agonist) are virus-infected diseases, wherein the virus is selected from Dengue virus, yellow fever virus, West Nile virus, Japanese encephalitis virus, tick-borne encephalitis virus, Kunjin virus, Murray Valley encephalitis virus, Saint Louis encephalitis virus, omsk haemorrhagic fever virus, bovine viral diarrhea virus, Zika virus, HIV, HBV, HCV, HPV, RSV, SARS, and influenza virus.

It is also an object of the present disclosure to provide a method for preventing and/or treating diseases at least partially mediated by a TLR7 agonist (preferably diseases mediated by a TLR7 agonist), which comprises administering to a patient a prophylactically and/or therapeutically effective amount of the compound, and the stereoisomer, the optical isomer, the pharmaceutically acceptable salt, the prodrug, and the solvate thereof of the present disclosure, or the pharmaceutical composition of the present disclosure.

### Definitions

The term “optional” or “optionally” means that the subsequently described event or circumstance may, but not necessarily, occur, and the description includes instances where the event or circumstance occurs and instances where it does not.

Unless otherwise specified, the term “alkyl” refers to a monovalent saturated aliphatic hydrocarbon group, i.e., a linear or branched group containing 1 to 20 carbon atoms, preferably containing 1 to 10 carbon atoms (i.e., C₁₋₁₀ alkyl), further preferably containing 1 to 8 carbon atoms (C₁₋₈ alkyl), and more preferably containing 1 to 6 carbon atoms (i.e., C₁₋₆ alkyl), for example, “C₁₋₆ alkyl” means that the group is alkyl and the number of carbon atoms on the carbon chain is between 1 and 6 (specifically 1, 2, 3, 4, 5, or 6). Non-limiting examples of the alkyl include, but are not limited to, methyl, ethyl, n-propyl, isopropyl, n-butyl, isobutyl, tert-butyl, sec-butyl, n-pentyl, neopentyl, 1,1-dimethylpropyl, 1,2-dimethylpropyl, 2,2-dimethylpropyl, 1-ethylpropyl, 2-methylbutyl, 3-methylbutyl, n-hexyl, n-heptyl, n-octyl, and the like.

Unless otherwise specified, the term “alkenyl” refers to a linear or branched unsaturated aliphatic hydrocarbon group consisting of carbon and hydrogen atoms having at least one double bond. The alkenyl may contain 2 to 20 carbon atoms, preferably 2 to 10 carbon atoms (i.e., C₂₋₁₀ alkenyl), further preferably 2 to 8 carbon atoms (C₂₋₈ alkenyl), more preferably 2 to 6 carbon atoms (i.e., C₂₋₆ alkenyl), 2 to 5 carbon atoms (i.e., C₂₋₅ alkenyl), 2 to 4 carbon atoms (i.e., C₂₋₄ alkenyl), 2 to 3 carbon atoms (i.e., C₂₋₃ alkenyl), 2 carbon atoms (i.e., C₂ alkenyl). For example, “C₂₋₆ alkenyl” means that the group is alkenyl and the number of carbon atoms on the carbon chain is between 2 and 6 (specifically 2, 3, 4, 5, or 6). Non-limiting examples of the alkenyl group include, but are not limited to, ethenyl, 1-propenyl, 2-propenyl, 1-butenyl, isobutenyl, 1,3-butadienyl, and the like. Unless otherwise specified, the term “alkynyl” refers to a linear or branched unsaturated aliphatic hydrocarbon group consisting of carbon and hydrogen atoms having at least one triple bond. The alkynyl may contain 2 to 20 carbon atoms, preferably 2 to 10 carbon atoms (i.e., C₂₋₁₀ alkynyl), further preferably 2 to 8 carbon atoms (C₂₋₈ alkynyl), more preferably 2 to 6 carbon atoms (i.e., C₂₋₆ alkynyl), 2 to 5 carbon atoms (i.e., C₂₋₅ alkynyl), 2 to 4 carbon atoms (i.e., C₂₋₄ alkynyl), 2 to 3 carbon atoms (i.e., C₂₋₃ alkynyl), 2 carbon atoms (i.e., C₂ alkynyl). For example, “C₂₋₆ alkynyl” means that the group is alkynyl and the number of carbon atoms on the carbon chain is between 2 and 6 (specifically 2, 3, 4, 5, or 6). Non-limiting examples of alkynyl include, but are not limited to, ethynyl, 1-propynyl, 2-propynyl, 1-butynyl, and the like.

Unless otherwise specified, the term “cycloalkyl” refers to a monocyclic saturated aliphatic hydrocarbon group containing a specific number of carbon atoms, preferably containing 3 to 12 carbon atoms (i.e., C₃₋₁₂ cycloalkyl), more preferably containing 3 to 10 carbon atoms (C₃₋₁₀ cycloalkyl), and further preferably containing 3 to 6 carbon atoms (C₃₋₆ cycloalkyl), 4 to 6 carbon atoms (C₄₋₆ cycloalkyl), or 5 to 6 carbon atoms (C₅₋₆ cycloalkyl). Non-limiting examples of the cycloalkyl include, but are not limited to, cyclopropyl, cyclobutyl, cyclopentyl, cyclohexyl, methylcyclopropyl, 2-ethyl-cyclopentyl, dimethylcyclobutyl, and the like.

Unless otherwise specified, the term “alkoxy” refers to —O-alkyl, wherein the alkyl is defined as above, i.e., the alkyl contains 1 to 20 carbon atoms, preferably 1 to 10 carbon atoms, more preferably 1 to 8 carbon atoms, and further more preferably 1 to 6 carbon atoms (specifically 1, 2, 3, 4, 5, or 6). Examples of the alkoxy include, but are not limited to, methoxy, ethoxy, propoxy, isopropoxy, butoxy, 1-methylpropoxy, 2-methylpropoxy, tert-butoxy, pentyloxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, and the like.

Unless otherwise specified, the term “halogen” or “halo” refers to F, Cl, Br, or I. The term “haloalkyl” means that one, two, or more hydrogen atoms or all hydrogen atoms in the alkyl defined as above are substituted with halogen. Examples of the haloalkyl include, but are not limited to, CCl₃, CF₃, CHCl₂, CH₂Cl, CH₂Br, CH₂I, CH₂CF₃, CF₂CF₃, and the like.

Unless otherwise specified, the term “heterocyclyl” refers to a saturated or partially unsaturated monocyclic, bicyclic, or polycyclic ring hydrocarbon substituent (e.g., 3,7-diazabicyclo[3.3.0]octane ring, etc.), which is a non-aromatic structure, and also includes some rings in the polycyclic ring that are aromatic structures (e.g., 1,2,3,4-tetrahydroisoquinoline ring, etc.). The heterocyclyl contains 3 to 20 ring atoms, wherein 1, 2, 3, or more ring atoms are selected from N, O, and S, and the remaining ring atoms are C. The heterocyclyl preferably contains 3 to 12 ring atoms, and further preferably 3 to 10 ring atoms, or 3 to 8 ring atoms, or 3 to 6 ring atoms, or 4 to 6 ring atoms, or 5 to 6 ring atoms. The number of the heteroatoms is preferably 1 to 4, and more preferably 1 to 3 (i.e. 1, 2, or 3). Examples of the monocyclic heterocyclyl include, but are not limited to, pyrrolidinyl, imidazolidinyl, tetrahydrofuranyl, dihydropyrrolyl, piperidinyl, piperazinyl, pyranyl, and the like. Polycyclic heterocyclyl includes spiro heterocyclyl, fused heterocyclyl, and bridged heterocyclyl.

Unless otherwise specified, “heterocycloalkyl” refers to a saturated “heterocyclyl” or “heterocyclic ring” defined as above, and the ring atoms are defined as above, i.e., the heterocycloalkyl contains 3 to 20 ring atoms (“3- to 20-membered heterocycloalkyl”). The number of heteroatoms is 1 to 4 (1, 2, 3, or 4), preferably 1 to 3 (1, 2, or 3), wherein the heteroatoms are each independently selected from N, O, and S. The heterocycloalkyl preferably contains 3 to 14 ring atoms (“3- to 14-membered heterocycloalkyl”), further preferably 3 to 10 ring atoms (“3- to 10-membered heterocycloalkyl”), still further preferably 3 to 8 ring atoms (“3- to 8-membered heterocycloalkyl”), still further preferably 4 to 7 ring atoms (“4- to 7-membered heterocycloalkyl”), still further preferably 5 to 10 ring atoms (“5- to 10-membered heterocycloalkyl”), and still further preferably 5 to 6 ring atoms (“5- to 6-membered heterocycloalkyl”). In certain embodiments, each example of the heterocycloalkyl is independently and optionally substituted, e.g., unsubstituted (an “unsubstituted heterocycloalkyl”) or substituted with one or more substituents (a “substituted heterocycloalkyl”). Some exemplary “heterocycloalkyl” have been given in the section of the “heterocyclyl” or “heterocyclic ring” above, and the heterocycloalkyl also includes, but is not limited to, azacyclopropyl, ozacyclopropyl, thiocyclopropyl, azacyclobutyl, oxacyclobutyl, thiocyclobutyl, tetrahydrofuranyl, oxacyclohexyl, piperidinyl, piperazinyl, morpholinyl, thiomorpholinyl, oxathianyl, oxazolidinyl, dioxanyl, dithiacyclohexyl, thiazolidinyl, pyrrolidinyl, pyrazolidinyl, imidazolinidinyl, and the like.

Unless otherwise specified, the term “carbocyclyl” or “carbocyclic ring” refers to a non-aromatic cyclic hydrocarbon group having 3 to 14 ring carbon atoms (“C₃₋₁₄ carbocyclyl”) and having no heteroatoms in the non-aromatic ring system. In some embodiments, the carbocyclyl group has 3 to 12 ring carbon atoms (“C₃₋₁₂ carbocyclyl”), or 4 to 12 ring carbon atoms (“C₄₋₁₂ carbocyclyl”), or 3 to 10 ring carbon atoms (“C₃₋₁₀ carbocyclyl”). In some embodiments, the carbocyclyl group has 3 to 8 ring carbon atoms (“C₃₋₈ carbocyclyl”). In some embodiments, the carbocyclyl group has 3 to 7 ring carbon atoms (“C₃₋₇ carbocyclyl”). In some embodiments, the carbocyclyl group has 4 to 6 ring carbon atoms (“C₄₋₆ carbocyclyl”). In some embodiments, the carbocyclyl group has 5 to 10 ring carbon atoms (“C₅₋₁₀ carbocyclyl”), or 5 to 7 ring carbon atoms (“C₅₋₇ carbocyclyl”). Exemplary C₃₋₆ carbocyclyl groups include, but are not limited to, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, but are not limited to, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptyl (C₇), bicyclo[2.2.2]octyl (C₈), and the like. Exemplary C₃₋₁₀ carbocyclyl groups include, but are not limited to, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthyl (C₁₀), spiro[4.5]decyl (C₁₀), and the like. As illustrated by the examples described above, in certain embodiments, the carbocyclyl group is monocyclic (“monocyclic carbocyclyl”) or is a fused ring system (fused cyclyl), bridged ring system (bridged cyclyl), or spiro-fused (spirocyclyl) ring system, such as a bicyclic ring system (“bicyclic carbocyclyl”), and may be saturated or partially unsaturated. The “carbocyclyl” also includes ring systems in which the carbocycle ring defined as above is fused by one or more aryl or heteroaryl groups, with the attachment site being on the carbocycle ring, and in such cases, the number of carbons is still indicative of the number of carbons in the carbocycle system. In certain embodiments, each example of the carbocyclyl group is independently and optionally substituted, e.g., unsubstituted (an “unsubstituted carbocyclyl”) or substituted with one or more substituents (a “substituted carbocyclyl”). In certain embodiments, the carbocyclyl group is an unsubstituted C₃₋₁₀ carbocyclyl. In certain embodiments, the carbocyclyl group is a substituted C₃₋₁₀ carbocyclyl. Unless otherwise specified, “cycloalkenyl” refers to a system composed of subgroups monocyclic hydrocarbon ring, bicyclic hydrocarbon ring and spiro-hydrocarbon ring, however, the system is unsaturated, i.e., at least one C—C double bond is present but no aromatic system. The cycloalkenyl preferably contains 3 to 12 carbon atoms (i.e., C₃₋₁₂ cycloalkenyl), more preferably 3 to 10 carbon atoms (C₃₋₁₀ cycloalkenyl), and further preferably 3 to 6 carbon atoms (C₃₋₆ cycloalkenyl), 4 to 6 carbon atoms (C₄₋₆ cycloalkenyl), or 5 to 6 carbon atoms (C₅₋₆ cycloalkenyl).

Unless otherwise specified, the term “fused ring” refers to a saturated or partially unsaturated non-aromatic bicyclic or polycyclic system formed by two or more cyclic structures that share two adjacent atoms with each other, including fused carbocyclyl and fused heterocyclyl, the “fused heterocyclyl” optionally containing one or more heteroatoms independently selected from oxygen, nitrogen, and sulfur.

Unless otherwise specified, the term “aryl” refers to a monocyclic, bicyclic and tricyclic aromatic carbocyclic system containing 6 to 16 carbon atoms, or 6 to 14 carbon atoms, or 6 to 12 carbon atoms, or 6 to 10 carbon atoms, preferably 6 to 10 carbon atoms, and the term “aryl” are used interchangeably with the term “aromatic cyclyl”. Examples of the aryl group may include, but are not limited to, phenyl, naphthyl, anthryl, phenanthryl, pyrenyl, and the like.

Unless otherwise specified, the term “heteroaryl” refers to an aromatic monocyclic or polycyclic ring system containing a 5- to 12-membered structure, or preferably a 5- to 10-membered structure, or a 5- to 8-membered structure, and more preferably a 5- to 6-membered structure, wherein 1, 2, 3, or more ring atoms are heteroatoms and the remaining atoms are carbon, the heteroatoms are independently selected from O, N, and S, and the number of the heteroatoms is preferably 1, 2, or 3. Examples of the heteroaryl include, but are not limited to, furanyl, thienyl, oxazolyl, thiazolyl, isoxazolyl, oxadiazolyl, thiadiazolyl, pyrrolyl, pyrazolyl, imidazolyl, triazolyl, tetrazolyl, pyridyl, pyrimidinyl, pyrazinyl, pyridazinyl, thiadiazolyl, triazinyl, phthalazinyl, quinolyl, isoquinolyl, pteridinyl, purinyl, indolyl, isoindolyl, indazolyl, benzofuranyl, benzothienyl, benzopyridyl, benzopyrimidinyl, benpyrazinyl, benzimidazolyl, benzophthalizinyl, pyrrolo[2,3-b]pyridyl, imidazo[1,2-a]pyridyl, pyrazolo[1,5-a]pyridinyl, pyrazolo[1,5-a]pyrimidinyl, imidazo[1,2-b]pyridazinyl, [1,2,4]triazolo[4,3-b]pyridazinyl, [1,2,4]triazolo[1,5-a]pyrimidinyl, [1,2,4]triazolo[1,5-a]pyridinyl, and the like.

Unless otherwise specified, the term “pharmaceutically acceptable salt” refers to a salt which is, within the scope of sound medical judgment, suitable for use in contact with the tissues of mammals, especially humans, without excessive toxicity, irritation, allergic response and the like and commensurate with a reasonable benefit/risk ratio. The salts may be prepared in situ during the final separation and purification of the compounds of the present disclosure, or prepared alone by reacting a free base or a free acid with a suitable reagent.

Unless otherwise specified, the term “solvate” means a physical association of the compound of the present disclosure with one or more solvent molecules (whether organic or inorganic). The physical association includes hydrogen bonding. In certain cases, the solvate can be isolated, for example, when one or more solvent molecules are incorporated into the crystal lattice of a crystalline solid. The solvent molecules in the solvate may be present in a regular arrangement and/or a disordered arrangement. The solvate may contain a stoichiometric or non-stoichiometric amount of solvent molecules. The “solvate” encompasses both solution phase and isolatable solvates. Exemplary solvates include, but are not limited to, hydrates, ethanolates, methanolates, and isopropanolates. Solvation methods are well known in the art.

Unless otherwise specified, the term “isotopically labeled analog” or “isotopic derivative” refers to a molecule in the compound of the present disclosure that is isotopically labeled, thereby providing an isotopically labeled analog that may have improved pharmacological activity. Isotopes commonly used as isotopic labels are hydrogen isotopes: ²H and ³H; carbon isotopes: ¹¹C, ¹³C, and ¹⁴C; chlorine isotopes: ³⁵Cl and ³⁷Cl; fluorine isotope: ¹⁸F; iodine isotopes: ¹²³I and ¹²⁵I; nitrogen isotopes: ¹³N and ¹⁵N; oxygen isotopes: ¹⁵O, ¹⁷O, and ¹⁸O; and sulfur isotope: ³⁵S. These isotopically labeled compounds can be used to research the distribution of pharmaceutical molecules in tissues. Particularly, deuterium ³H and carbon ¹³C are more widely used due to their ease of labeling and ease of detection. The substitution with certain heavy isotopes, such as deuterium (²H), can enhance the stability of metabolism and prolong the half-life to achieve the purpose of reducing the dose and provide therapeutic advantages. Isotopically labeled compounds are generally synthesized starting from labeled starting materials and synthesized using known synthetic techniques in the same way as non-isotopically labeled compounds. In general, the compounds of the present disclosure include isotopic derivatives thereof (e.g., deuterides).

Unless otherwise specified, the term “optical isomer” refers to substances that have completely identical molecular structures and similar physicochemical properties, but different optical rotation.

Unless otherwise specified, the term “stereoisomer” refers to compounds having the same chemical structure but different spatial arrangements of the atoms or groups. Stereoisomers include enantiomers, diastereoisomers, conformers (rotamers), geometric isomers (cis/trans isomers), atropisomers, and the like. Any resulting mixture of stereoisomers may be separated into pure or substantially pure geometric isomers, enantiomers and diastereoisomers depending on differences in the physicochemical properties of the components, for example, by chromatography and/or fractional crystallization.

Unless otherwise specified, the term “tautomer” refers to structural isomers having different energies that are interconvertible by a lower energy barrier. If a tautomer is possible (e.g., in solution), the chemical equilibrium of the tautomer can be reached. For example, a proton tautomer (also known as a prototropic tautomer) includes interconversion by proton migration, such as keto-enol isomerism and imine-enamine isomerism. A valence tautomer includes the interconversion by recombination of some bonding electrons.

Unless otherwise indicated, the structural formulas described herein include all isomeric forms (e.g., enantiomers, diastereoisomers, and geometric isomers (or conformational isomers)): such as R and S configuration containing an asymmetric center, (Z) and (E) isomers of double bonds, and (Z) and (E) conformational isomers. Thus, individual stereochemical isomers or mixtures of enantiomers, diastereoisomers, or geometric isomers (or conformational isomers) thereof of the compounds of the present disclosure are within the scope of the present disclosure.

Unless otherwise specified, the term “prodrug” refers to a drug that is converted into a parent drug in vivo. The prodrugs are often useful because, in some cases, they can be easier to be administered than the parent drug. For example, they can be bioavailable by oral administration, whereas the parent drug cannot. The prodrugs also have improved solubility in pharmaceutical compositions as compared to the parent drug. An example of the prodrug, but not limited thereto, can be any compound of formula I that is administered as an ester (“prodrug”) to facilitate delivery across the cell membrane, wherein water solubility is detrimental to mobility, but once it enters the cell, the water solubility is beneficial, and it is subsequently metabolically hydrolyzed into carboxylic acid, i.e., an active entity. Another example of the prodrug can be a short peptide (polyamino acid) binding to an acid group, wherein the peptide is metabolized to show the active moiety.

Unless otherwise specified, the term “optionally substituted” means that the hydrogen at the substitutable site of the group is unsubstituted or substituted with one or more substituents, preferably substituents selected from: halogen, hydroxyl, sulfhydryl, cyano, nitro, amino, azido, oxo, carboxyl, C₂₋₆ alkenyl, C₂₋₆ alkynyl, C₁₋₆ alkyl, C₁₋₆ alkoxy, C₃₋₁₀ cycloalkyl, C₃₋₁₀ cycloalkylsulfonyl, 3- to 10-membered heterocycloalkyl, C₆₋₁₄ aryl, and 5- to 10-membered heteroaryl ring group, wherein the C₂₋₆ alkenyl, C₂₋₆ alkynyl, C₁₋₆ alkyl, C₁₋₆ alkoxy, C₃₋₁₀ cycloalkyl, C₃₋₁₀ cycloalkylsulfonyl, 3- to 10-membered heterocycloalkyl, C₆₋₁₄ aryl, or 5- to 10-membered heteroaryl ring group can be optionally substituted with one or more substituents selected from halogen, hydroxyl, amino, cyano, C₁₋₆ alkyl, and C₁₋₆ alkoxy, and the oxo means that two H at the same substitution site are replaced by the same O to form a double bond.

### Beneficial Effects of the Present Disclosure

The present disclosure designs a compound with a novel structure, and provides a new direction for the development of TLR7 agonist drugs. The research on the agonistic activity of the human-detived receptor TLR7 shows that these compounds have a stronger agonistic effect on the human-detived receptor TLR7 and can be used as prospect compounds for preventing and/or treating at least partial diseases mediated by TLR7 agonists. In addition, the present disclosure researches a specific synthetic method, and the synthetic method has the advantages of simple process and convenient operation, and is beneficial to large-scale industrial production and application.

### DETAILED DESCRIPTION

The present disclosure will be further illustrated with reference to the following specific examples. It should be understood that these examples are merely intended to illustrate the present disclosure rather than limit the scope of the present disclosure. Experimental procedures without specified conditions in the following examples are generally conducted according to conventional conditions or according to conditions recommended by the manufacturer. Unless otherwise defined, all technical and scientific terms used herein have the same meaning as commonly understood by those of ordinary skill in the art. In addition, any methods and materials similar or equivalent to those described herein can be used in the methods of the present disclosure. The preferred embodiments and materials described herein are for illustrative purposes only.

The compound structure of the present disclosure is determined by nuclear magnetic resonance (NMR) and/or liquid chromatography-mass spectrometry (LC-MS) and/or high-performance liquid chromatography (HPLC). The instrument used for NMR is Bruker AVANCE NEO 400 MHz, the instrument used for LC-MS is LC-MS WATERS ACQUITY UPLC H-Class PLUS or/and SQD2, and the instrument used for HPLC is WATERS ACQUITYUPLC or/and Agilent 1260. Starting materials in the examples of the present disclosure are known and commercially available, or may be synthesized by using or according to methods known in the art.

### Example 1: Preparation of 4-amino-1-(3-(pyrrolidin-1-ylmethyl)benzyl)-1,3-dihydro-2H-imidazo[4,5-c]quinolin-2-one

### Step 1: Preparation of 3-nitroquinoline-2,4-diol

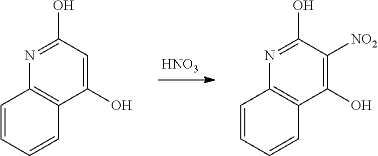 [SMILES: Oc1cc(O)c2ccccc2n1 ; "HNO3" ; O=[N+]([O-])c1c(O)nc2ccccc2c1O]

The starting material quinoline-2,4-diol (14.0 g, 86.9 mmol, 1 eq) was dissolved in nitric acid (85 mL, 68%). The reaction mixture was stirred at 75° C. for 30 min. After the reaction was completed, as detected by TLC, the reaction mixture was cooled and then added dropwise to ice water (100 mL). The mixture was filtered and dried to give the target compound (17.3 g, 96.7%).

### Step 2: Preparation of 2,4-dichloro-3-nitroquinoline

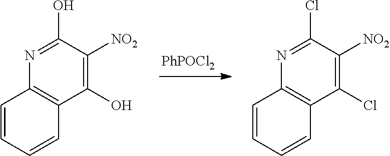 [SMILES: "PhPOCl2" ; O=[N+]([O-])c1c(O)nc2ccccc2c1O ; O=[N+]([O-])c1c(Cl)nc2ccccc2c1Cl]

The starting material 3-nitroquinoline-2,4-diol (17.3 g, 84.0 mmol, 1 eq) was dissolved in phenylphosphonic dichloride (100 mL). The reaction mixture was stirred at 140° C. for 3 h. After the reaction was completed, as detected by TLC, the reaction mixture was added dropwise to ice water (200 mL). The mixture was extracted with ethyl acetate (200 mL×2). The combined organic phases were dried over anhydrous sodium sulfate, then filtered and concentrated to give a crude product, which was separated and purified by flash chromatography (PE:EA=100:0 to 80:20) to give the target compound (14.7 g, 72.0%). ¹H NMR (400 MHz, CDCl₃): δ 8.28 (d, J=8.4 Hz, 1H), 8.12 (d, J=8.4 Hz, 1H), 7.97-7.93 (m, 1H), 7.80-7.83 (m, 1H).

### Step 3: Preparation of 2-chloro-3-nitro-N-(3-(pyrrolidin-1-ylmethyl)benzyl)quinoline-4-amine

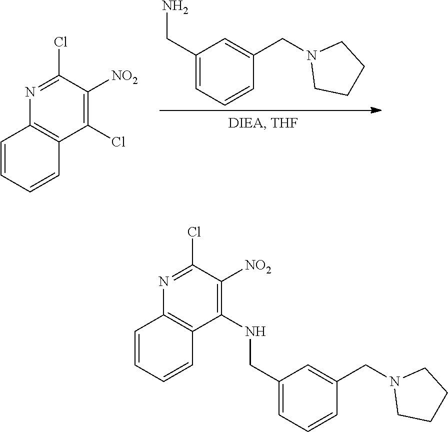 [SMILES: O=[N+]([O-])c1c(Cl)nc2ccccc2c1Cl ; NCc1cccc(CN2CCCC2)c1 ; O=[N+]([O-])c1c(Cl)nc2ccccc2c1NCc1cccc(CN2CCCC2)c1]

The starting material 2,4-dichloro-3-nitroquinoline (2.00 g, 8.23 mmol, 1 eq) was dissolved in tetrahydrofuran (15 mL), and (3-(pyrrolidin-1-ylmethyl)phenyl)methylamine (1.57 g, 8.23 mmol, 1 eq) and N,N-diisopropylethylamine (1.38 g, 10.70 mmol, 1.3 eq) were added. The reaction mixture was stirred at 25° C. for 18 h. After the reaction was completed, as detected by LC-MS, the reaction mixture was concentrated to remove tetrahydrofuran to give a crude product, and then water (50 mL) was added. The mixture was extracted with ethyl acetate (50 mL×2). The combined organic phases were dried over anhydrous sodium sulfate, then filtered and concentrated to give a crude product, which was separated and purified by flash chromatography (DCM:MeOH=100:0 to 95:5) to give the target compound (2.70 g, 82.3%). LC-MS: [M+H]⁺=397.03.

### Step 4: Preparation of 2-chloro-N⁴-(3-(pyrrolidin-1-ylmethyl)benzyl)quinoline-3,4-diamine

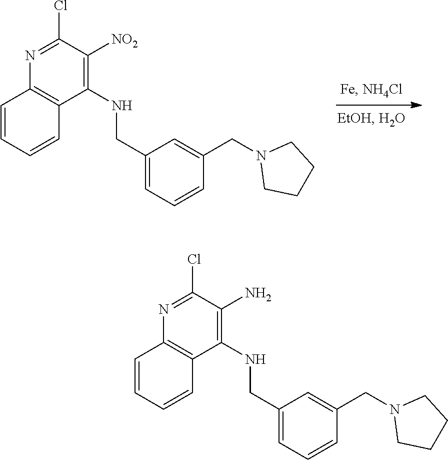 [SMILES: O=[N+]([O-])c1c(Cl)nc2ccccc2c1NCc1cccc(CN2CCCC2)c1 ; Nc1c(Cl)nc2ccccc2c1NCc1cccc(CN2CCCC2)c1]

The starting material 2-chloro-3-nitro-N-(3-(pyrrolidin-1-ylmethyl)benzyl)quinolin-4-amine (2.7 g, 6.80 mmol, 1 eq) was dissolved in ethanol (10 mL) and water (10 mL), and iron powder (1.90 g, 34.0 mmol, 5 eq) and ammonium chloride (1.82 g, 34.0 mmol, 5 eq) were added. The reaction mixture was stirred at 80° C. for 2 h. After the reaction was completed, as detected by LC-MS, the reaction mixture was filtered and concentrated to give a crude product, which was separated and purified by flash chromatography (DCM:MeOH=100:0 to 93:7) to give the target compound (790 mg, 31.7%). LC-MS: [M+H]⁺=367.02.

### Step 5: Preparation of 4-chloro-1-(3-(pyrrolidin-1-ylmethyl)benzyl)-1,3-dihydro-2H-imidazo[4,5-c]quinolin-2-one

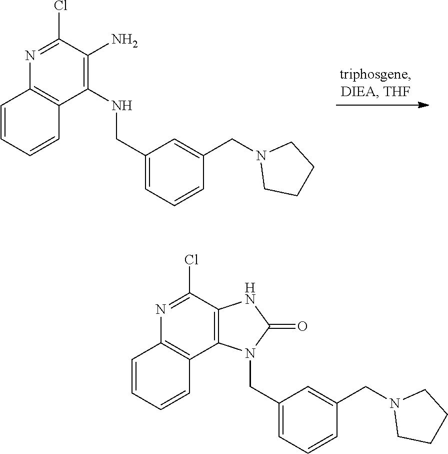 [SMILES: Nc1c(Cl)nc2ccccc2c1NCc1cccc(CN2CCCC2)c1 ; O=c1[nH]c2c(Cl)nc3ccccc3c2n1Cc1cccc(CN2CCCC2)c1]

The starting material 2-chloro-N⁴-(3-(pyrrolidin-1-ylmethyl)benzyl) quinoline-3,4-diamine (340 mg, 0.93 mmol, 1 eq) was dissolved in tetrahydrofuran (1 mL), and triphosgene (275 mg, 0.93 mmol, 1 eq) and N,N-diisopropylethylamine (359 mg, 2.78 mmol, 3 eq) were added. The reaction mixture was stirred at 25° C. for 18 h. After the reaction was completed, as detected by LC-MS, the reaction mixture was concentrated to give a solid residue. The solid residue was stirred with dichloromethane (2 mL) at 25° C. for 30 min and filtered to give the target product (310 mg, 85.1%). LC-MS: [M+H]⁺=393.03

### Step 6: Preparation of 4-amino-1-(3-(pyrrolidin-1-ylmethyl)benzyl)-1,3-dihydro-2H-imidazo[4,5-c]quinolin-2-one

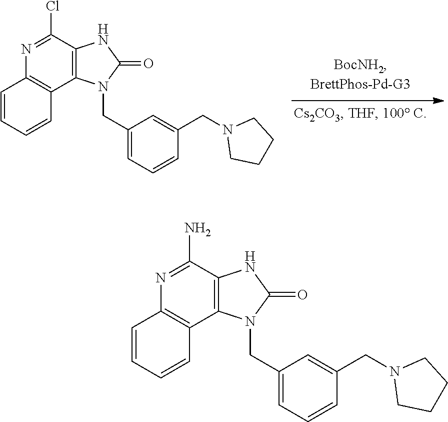 [SMILES: O=c1[nH]c2c(Cl)nc3ccccc3c2n1Cc1cccc(CN2CCCC2)c1 ; Nc1nc2ccccc2c2c1[nH]c(=O)n2Cc1cccc(CN2CCCC2)c1]

The starting material 4-chloro-1-(3-(pyrrolidin-1-ylmethyl)benzyl)-1,3-dihydro-2H-imidazo[4,5-c]quinolin-2-one (70 mg, 0.18 mmol, 1 eq) was dissolved in tetrahydrofuran (1 mL), and tert-butyl carbamate (208 mg, 1.78 mmol, 10 eq), cesium carbonate (174 mg, 534 μmol, 3 eq), and BrettPhos-Pd-G3 (methenesulfonato2-dicyclohexylphosphino-3,6-dimethoxy-2′-4′-6′-tri-i-propyl-1,1′-bipheny)(2′-amino-1,1′-biphenyl-2-yl)palladium(II)) (64 mg, 71.3 μmol, 0.4 eq) were added respectively under nitrogen atmosphere. The reaction mixture was stirred at 100° C. for 5 h. After the reaction was completed, as detected by LC-MS, the reaction mixture was concentrated to remove tetrahydrofuran, and then water (5 mL) was added. The mixture was extracted with ethyl acetate (10 mL×2). The organic phases were combined, dried over anhydrous sodium sulfate, filtered and concentrated to give a crude product, which was separated and purified by Prep-HPLC (0.01% aqueous HCOOH solution, MeCN) to give the target compound (8.92 mg, 13.4%). LC-MS: [M+H]⁺=374.10; ¹H NMR (400 MHz, CD₃OD): δ 8.42 (brs, 2H), 7.77 (d, J=8.4 Hz, 1H), 7.66 (d, J=8.4 Hz, 1H), 7.53-7.51 (m, 2H), 7.46-7.43 (m, 2H), 7.29 (s, 1H), 7.14-7.13 (m, 1H), 5.66 (s, 2H), 4.26 (s, 2H), 3.09 (br s, 4H), 1.96 (brs, 4H).

### Example 2: Preparation of 1-(3-(pyrrolidin-1-ylmethyl)benzyl)-1H-imidazo[4,5-c]quinoline-4-amine

### Step 1: Preparation of 4-chloro-1-(3-(pyrrolidin-1-ylmethyl)benzyl)-1H-imidazo[4,5-c]quinoline

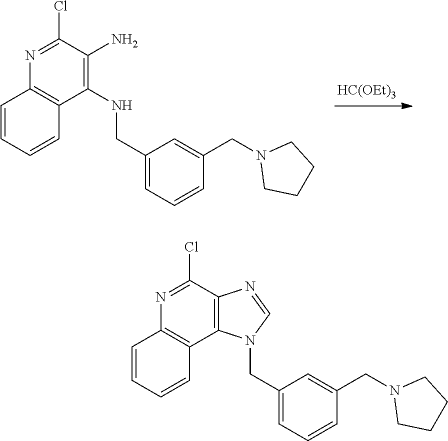

The starting material 2-chloro-N⁴-(3-(pyrrolidin-1-ylmethyl)benzyl)quinoline-3,4-diamine (1.00 g, 2.73 mmol, 1.00 eq) was dissolved in triethyl orthoformate (10 mL). The reaction mixture was stirred at 80° C. for 16 h under nitrogen atmosphere. After the reaction was completed, as detected by LC-MS, the reaction mixture was concentrated to give a crude product, the solid was slurried with dichloromethane (10 mL) for 30 min and filtered, and the filter cake was the target compound (720 mg, 70.1%). LC-MS: [M+H]⁺=377.00; ¹H NMR (400 MHz, CD₃OD): δ 8.56 (s, 1H), 8.06 (d, J=9.2 Hz, 2H), 7.69 (d, J=9.2 Hz, 1H), 7.54-7.49 (m, 2H), 7.46-7.44 (m, 2H), 7.09 (s, 1H), 6.11 (s, 2H), 4.22 (s, 2H), 2.99 (brs, 4H), 1.92 (brs, 4H).

### Step 2: Preparation of 1-(3-(pyrrolidin-1-ylmethyl)benzyl)-1H-imidazo[4,5-c]quinoline-4-amine

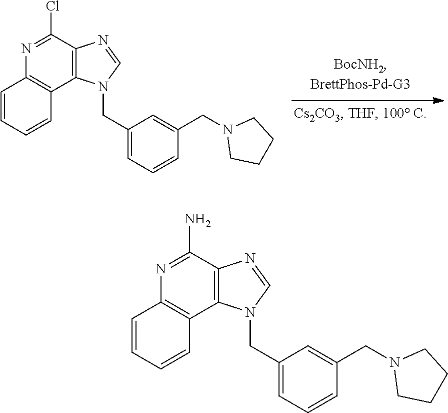 [SMILES: Clc1nc2ccccc2c2c1ncn2Cc1cccc(CN2CCCC2)c1 ; Nc1nc2ccccc2c2c1ncn2Cc1cccc(CN2CCCC2)c1]

The starting material 4-chloro-1-(3-(pyrrolidin-1-ylmethyl)benzyl)-1H-imidazo[4,5-c]quinoline (658 mg, 1.74 mmol, 1.00 eq) was dissolved in tetrahydrofuran, and tert-butyl carbamate (245 mg, 2.09 mmol, 1.20 eq), cesium carbonate (1.71 g, 5.23 mmol, 3.00 eq), and BrettPhos-Pd-G3 (methanesulfonato2-dicyclohexylphosphino-3,6-dimethoxy-2′-4′-6′-tri-i-propyl-1,1′-biphenyl)(2′-amino-1,1′-biphenyl-2-yl)palladium(II)) (158 mg, 0.174 mmol, 0.12 eq) were added under nitrogen atmosphere. The reaction mixture was stirred at 100° C. for 5 h. After the reaction was completed, as detected by LC-MS, the reaction system was concentrated to give a solid, the solid was slurried with ethyl acetate (8 mL) for 30 min and filtered, and the filter cake was a crude product (280 mg, 44.90%). 50.00 mg of the crude product was separated and purified by Prep-HPLC (0.01% aqueous FA solution, MeCN) to give the target compound (10.5 mg, 20.9%). LC-MS: [M+H]⁺=358.10; ¹H NMR (400 MHz, CD₃OD): δ 8.48 (s, 2H), 8.37 (s, 1H), 7.82 (d, J=8.4 Hz, 1H), 7.70 (d, J=8.4 Hz, 1H), 7.51-7.48 (m, 2H), 7.42-7.39 (m, 2H), 7.22-7.19 (m, 1H), 7.08 (s, 1H), 6.00 (s, 2H), 4.14 (s, 2H), 2.95-2.94 (m, 4H), 1.92-1.87 (m, 4H).

### Example 3: Preparation of (3-((4-amino-1H-imidazo[4,5-c]quinolin-1-yl)methyl)phenyl)(pyrrolidin-1-yl)methanone

### Step 1: Preparation of (3-(chloromethyl)phenyl)(pyrrolidin-1-yl)methanone

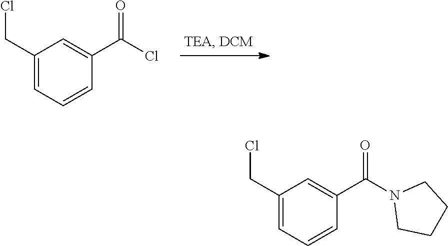 [SMILES: O=C(Cl)c1cccc(CCl)c1 ; O=C(c1cccc(CCl)c1)N1CCCC1]

The starting material 3-(chloromethyl)benzoyl chloride (500 mg, 2.64 mmol, 1.0 eq) was dissolved in dichloromethane (5 mL), followed by the addition of pyrrolidine (188 mg, 2.64 mmol, 1.0 eq) and triethylamine (803 mg, 7.93 mmol, 3.0 eq). The reaction mixture was stirred at 25° C. for 2 h. After the reaction was completed, as detected by TLC, water (30 mL) was added to the reaction mixture for dilution, and the mixture was filtered. The mixture was then extracted with dichloromethane (20 mL×3). The combined organic phases were washed with saturated brine (20 mL), dried over anhydrous sodium sulfate, filtered and concentrated to give the target compound (500 mg, 84.5%). The product was used directly in the next step. LC-MS: [M+H]⁺=224.04.

### Step 2: Preparation of (3-(aminomethyl)phenyl)(pyrrolidin-1-yl)methanone

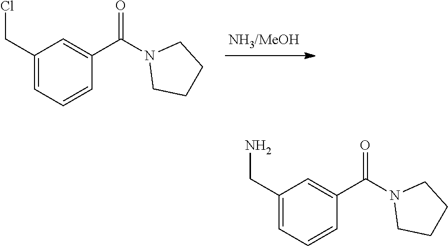 [SMILES: O=C(c1cccc(CCl)c1)N1CCCC1 ; NCc1cccc(C(=O)N2CCCC2)c1]

The starting material (3-(chloromethyl)phenyl)(pyrrolidin-1-yl)methanone (600 mg, 2.68 mmol, 1.0 eq) was dissolved in a solution of ammonia in methanol (7 M, 1.37 mL). The reaction mixture was stirred at 25° C. for 16 h. After the reaction was completed, as detected by TLC, the reaction mixture was concentrated to give a crude product, which was separated and purified by flash chromatography (DCM:MeOH=50:1 to 10:1) to give the target compound (230 mg, 42.0%). LC-MS: [M+H]⁺=205.08.

### Step 3: Preparation of (3-(((2-chloro-3-nitroquinolin-4-yl)amino)methyl)phenyl)(pyrrolidin-1-yl)methanone

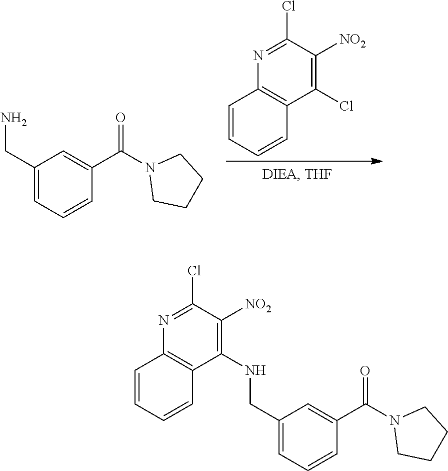 [SMILES: NCc1cccc(C(=O)N2CCCC2)c1 ; O=[N+]([O-])c1c(Cl)nc2ccccc2c1Cl ; O=C(c1cccc(CNc2c([N+](=O)[O-])c(Cl)nc3ccccc23)c1)N1CCCC1]

The starting materials (3-(aminomethyl)phenyl)(pyrrolidin-1-yl)methanone (1.31 g, 5.39 mmol, 1.0 eq), N,N-diisopropylethylamine (2.09 g, 16.2 mmol, 3.0 eq), and 2,4-dichloro-3-nitroquinoline (1.10 g, 5.39 mmol, 1.0 eq) were dissolved in tetrahydrofuran (20 mL). The reaction mixture was stirred at 80° C. for 16 h. After the reaction was completed, as detected by LC-MS, the reaction mixture was diluted with water (20 mL), and then extracted with dichloromethane (20 mL×4). The combined organic phases were washed with saturated brine (20 mL), dried over anhydrous sodium sulfate, filtered and concentrated to give a crude product, which was slurried (DCM:MeOH=20:1) to give the target compound (1.30 g, 58.1%). LC-MS: [M+H]⁺=411.01; ¹H NMR (400 MHz, DMSO-d₆): δ 8.57-8.54 (m, 2H), 1.87-1.86 (m, 2H), 7.70-7.68 (m, 1H), 7.40-7.39 (m, 2H), 7.36-7.35 (m, 1H), 4.50 (d, J=6.0 Hz, 2H), 3.42 (t, J=6.8 Hz, 2H), 3.24 (t, J=6.8 Hz, 2H), 1.84-1.72 (m, 4H).

### Step 4: Preparation of (3-(((3-amino-2-chloroquinolin-4-yl)amino)methyl)phenyl)(pyrrolidin-1-yl)methanone

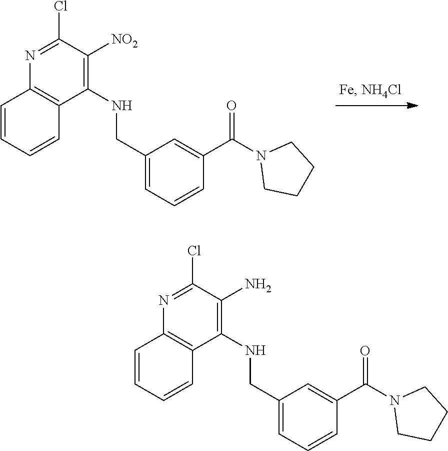 [SMILES: O=C(c1cccc(CNc2c([N+](=O)[O-])c(Cl)nc3ccccc23)c1)N1CCCC1 ; Nc1c(Cl)nc2ccccc2c1NCc1cccc(C(=O)N2CCCC2)c1]

The starting material (3-(((2-chloro-3-nitroquinolin-4-yl)amino)methyl)phenyl)(pyrrolidin-1-yl)methanone (1.25 g, 3.04 mmol, 1.0 eq) was dissolved in ethanol (15 mL) and water (5 mL), and iron powder (510 mg, 9.13 mmol, 3.0 eq) and ammonium chloride (488 mg, 9.13 mmol, 3.0 eq) were added. The reaction mixture was stirred at 80° C. for 20 h. After the reaction was completed, as detected by LC-MS, the reaction mixture was filtered. The filter cake was washed with ethanol (5 mL×4). The filtrate was concentrated to give a crude product, which was separated and purified by flash chromatography (DCM:MeOH=50:1 to 20:1) to give the target compound (1.02 g, 88.0%). LC-MS: [M+H]⁺=381.02.

### Step 5: Preparation of (3-((4-chloro-1H-imidazo[4,5-c]quinolin-1-yl)methyl)phenyl)(pyrrolidin-1-yl)methanone

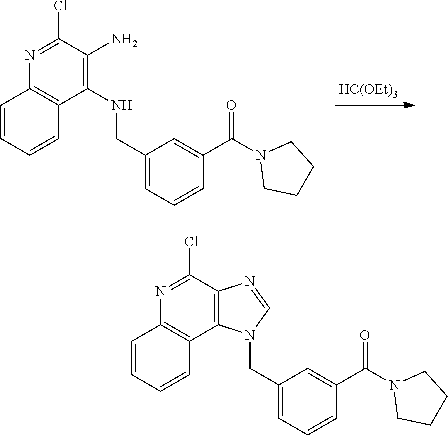

The starting material (3-(((3-amino-2-chloroquinolin-4-yl)amino)methyl)phenyl)(pyrrolidin-1-yl)methanone (1.02 g, 2.68 mmol, 1.0 eq) was dissolved in triethyl orthoformate (10 mL) and ethanol (10 mL). The reaction mixture was stirred at 80° C. for 16 h. After the reaction was completed, as detected by LC-MS, the reaction mixture was concentrated to give a crude product, which was slurried (DCM:EA=1:10) and purified to give the target compound (430 mg, 39.0%). LC-MS: [M+H]⁺=391.02; ¹H NMR (400 MHz, CDCl₃): δ 8.16 (d, J=7.6 Hz, 1H), 8.07 (s, 1H), 7.88 (d, J=7.6 Hz, 1H), 7.66-7.61 (m, 1H), 7.48-7.43 (m, 2H), 7.49 (J=7.6 Hz, 1H), 7.23 (s, 1H), 7.14 (d, J=7.6 Hz, 1H), 5.85 (s, 2H), 3.56 (t, J=6.6 Hz, 2H), 3.06 (t, J=6.6 Hz, 2H), 1.89-1.87 (m, 2H), 1.75-1.73 (m, 2H).

### Step 6: Preparation of (3-((4-amino-1H-imidazo[4,5-c]quinolin-1-yl)methyl)phenyl)(pyrrolidin-1-yl)methanone

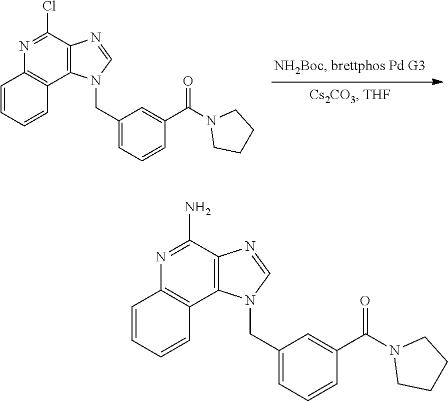 [SMILES: O=C(c1cccc(Cn2cnc3c(Cl)nc4ccccc4c32)c1)N1CCCC1 ; Nc1nc2ccccc2c2c1ncn2Cc1cccc(C(=O)N2CCCC2)c1]

The starting material (3-((4-chloro-1H-imidazo[4,5-c]quinolin-1-yl)methyl)phenyl)(pyrrolidin-1-yl)methanone (200 mg, 512 μcool, 1.0 eq), tert-butyl carbamate (71.9 mg, 614 μmol, 1.2 eq), and cesium carbonate (500 mg, 1.54 mmol, 3.0 eq) were dissolved in tetrahydrofuran (10 mL), and BrettPhos-Pd-G3 (methanesulfonato2-dicyclohexylphosphino-3,6-dimethoxy-2′-4′-6′-tri-i-propyl-1,1′-biphenyl)(2′-amino-1,1′-biphenyl-2-yl)palladium(II)) (46.4 mg, 51.2 μmol, 0.1 eq) was added. The reaction mixture was stirred at 100° C. for 16 h under nitrogen atmosphere. After the reaction was completed, as detected by LC-MS, the reaction mixture was diluted with water (20 mL), and then extracted with ethyl acetate (20 mL×3). The combined organic phases were washed with saturated brine (20 mL), dried over sodium sulfate, filtered and concentrated to give a crude product, which was separated and purified by Prep HPLC (0.01% aqueous HCOOH solution, MeCN) to give the target compound (55.9 mg, 28.8%). LC-MS: [M+H]⁺=372.10; ¹H NMR (400 MHz, DMSO-d₆): δ 8.43 (s, 1H), 8.23 (s, 1H), 7.83 (d, J=8.4 Hz, 1H), 7.59 (d, J=8.4 Hz, 1H), 7.39-7.36 (m, 3H), 7.21-7.18 (m, 2H), 7.08-7.04 (m, 3H), 5.97 (s, 2H), 3.37 (t, J=6.6 Hz, 2H), 3.07 (t, J=6.6 Hz, 2H), 1.80-1.77 (m, 2H), 1.69-1.66 (m, 2H).

### Example 4: Preparation of (3-((1H-imidazo[4,5-c]quinolin-1-yl)methyl)phenyl)(pyrrolidin-1-yl)methanone

### Step 1: Preparation of (3-((1H-imidazo[4,5-c]quinolin-1-yl)methyl)phenyl)(pyrrolidin-1-yl)methanone

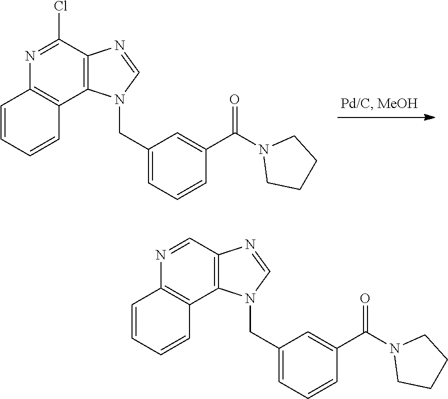

The starting material (3-((4-chloro-1H-imidazo[4,5-c]quinolin-1-yl)methyl)phenyl)(pyrrolidin-1-yl)methanone (40.0 mg, 102 μmol, 1.0 eq) was dissolved in methanol (1.5 mL), and palladium on carbon (20.0 mg, 5% purity) was added. The reaction mixture was stirred at 15° C. for 8 h under hydrogen atmosphere (15 psi). After the reaction was completed, as detected by LC-MS, the reaction mixture was filtered and concentrated to give a crude product, which was separated and purified by Prep HPLC (0.01% aqueous TFA solution, MeCN) to give the target compound (13.8 mg, 37.7%). LC-MS: [M+H]⁺=357.01; ¹H NMR (400 MHz, DMSO-d₆): δ 9.72 (s, 1H), 8.91 (s, 1H), 8.33 (d, J=8.4 Hz, 1H), 8.28 (d, J=8.4 Hz, 1H), 7.88-7.86 (m, 1H), 7.75-7.73 (m, 1H), 7.31-7.29 (m, 2H), 7.25 (s, 1H), 7.25-7.24 (m, 1H), 6.17 (s, 2H), 3.37 (t, J=6.8 Hz, 2H), 3.09 (t, J=6.8 Hz, 2H), 1.81-1.76 (m, 2H), 1.72-1.67 (m, 2H).

### Example 5: Preparation of (3-((4-hydroxy-1H-imidazo[4,5-c]quinolin-1-yl)methyl)phenyl)(pyrrolidin-1-yl)methanone

### Step 1: Preparation of (3-((4-hydroxy-1H-imidazo[4,5-c]quinolin-1-yl)methyl)phenyl)(pyrrolidin-1-yl)methanone

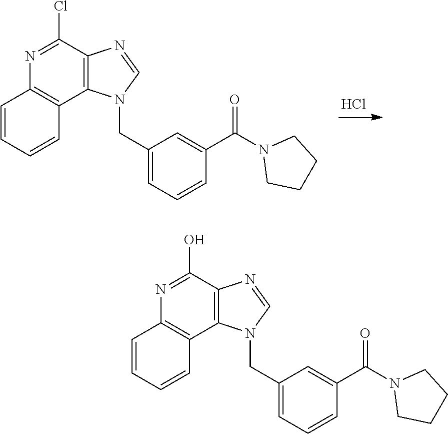

The starting material (3-((4-chloro-1H-imidazo[4,5-c]quinolin-1-yl)methyl)phenyl)(pyrrolidin-1-yl)methanone (80.0 mg, 205 μmol, 1.0 eq) was dissolved in hydrochloric acid (6 M, 2 mL). The reaction mixture was stirred at 100° C. for 2 h. After the reaction was completed, as detected by LC-MS, the reaction mixture was diluted with water (2 mL), and then adjusted to pH=8 with aqueous ammonia. The mixture was filtered, and the filter cake was separated and purified by Prep-HPLC (0.01% aqueous TFA solution, MeCN) to give the target compound (13.8 mg, 17.8%) in the form of a white solid. LC-MS: [M+H]⁺=373.01; ¹H NMR (400 MHz, DMSO-d₆): δ 11.62 (s, 1H), 8.32 (s, 1H), 7.75 (d, J=8.0 Hz, 1H), 7.43-7.37 (m, 4H), 7.19 (s, 2H), 7.06-7.02 (m, 1H), 5.93 (s, 2H), 3.40-3.38 (m, 2H), 3.09 (t, J=6.6 Hz, 2H), 1.82-1.78 (m, 2H), 1.71-1.67 (m, 2H).

### Example 6: Preparation of 4-amino-1-(3-(4-((methylamino)methyl)phenoxy)benzyl)-1,3-dihydro-2H-imidazo[4,5-c]quinolin-2-one

### Step 1: Preparation of tert-butyl (4-(4-(((2-chloro-3-nitroquinolin-4-yl)amino)methyl)phenoxy)benzyl)(methyl)carbamate

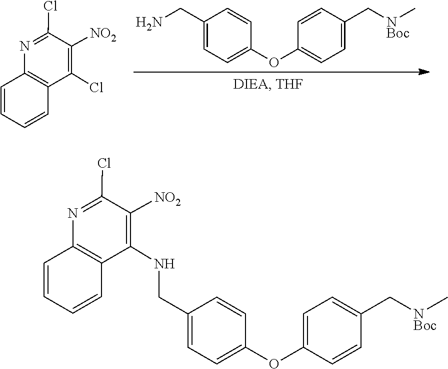 [SMILES: O=[N+]([O-])c1c(Cl)nc2ccccc2c1Cl ; CN(Cc1ccc(Oc2ccc(CN)cc2)cc1)C(=O)OC(C)(C)C ; CN(Cc1ccc(Oc2ccc(CNc3c([N+](=O)[O-])c(Cl)nc4ccccc34)cc2)cc1)C(=O)OC(C)(C)C]

The starting material 2,4-dichloro-3-nitroquinoline (1.00 g, 4.11 mmol, 1.0 eq) was dissolved in tetrahydrofuran (10 mL), and tert-butyl (4-(4-(aminomethyl)phenoxy)benzyl)(methyl)carbamate (1.41 g, 4.11 mmol, 1.0 eq) and N,N-diisopropylethylamine (1.60 g, 12.3 mmol, 3 eq) were added. The reaction mixture was stirred at 25° C. for 18 h. After the reaction was completed, as detected by LC-MS, the reaction mixture was concentrated to remove the solvent, and water (8 mL) was added. The mixture was extracted with ethyl acetate (10 mL×2), then dried over anhydrous sodium sulfate, filtered and concentrated to give a solid, which was the target product (2.20 g, 97.8%). The product was used directly in the next step. LC-MS: [M−56]⁺=493.00.

### Step 2: Preparation of tert-butyl (4-(4-(((3-amino-2-chloroquinolin-4-yl)amino)methyl)phenoxy)benzyl)(methyl)carbamate

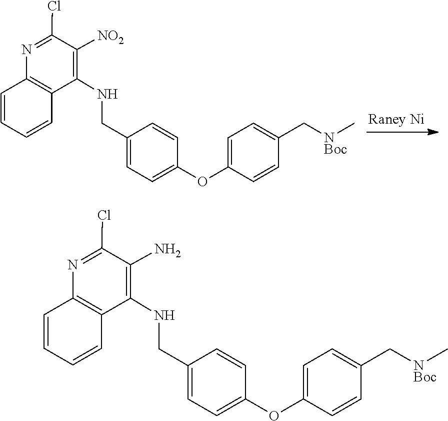 [SMILES: CN(Cc1ccc(Oc2ccc(CNc3c([N+](=O)[O-])c(Cl)nc4ccccc34)cc2)cc1)C(=O)OC(C)(C)C ; CN(Cc1ccc(Oc2ccc(CNc3c(N)c(Cl)nc4ccccc34)cc2)cc1)C(=O)OC(C)(C)C]

The starting material tert-butyl (4-(4-(((2-chloro-3-nitroquinolin-4-yl)amino)methyl)phenoxy)benzyl)(methyl)carbamate (2.20 g, 4.01 mmol, 1.0 eq) was dissolved in methanol (10 mL), and Raney nickel (343 mg) was added under nitrogen atmosphere. The reaction mixture was purged with nitrogen 3 times and then purged with hydrogen 3 times. The reaction mixture was stirred at 25° C. for 18 h under hydrogen atmosphere (15 psi). After the reaction was completed, as detected by LC-MS, the reaction mixture was filtered, and the filtrate was concentrated to give a solid, which was separated and purified by flash chromatography (DCM:MeOH=100:0 to 91:9) to give the target compound (1.07 g, 51.5%). LC-MS: [M+H]⁺=519.09; ¹H NMR (400 MHz, CDCl₃): δ 7.84 (d, J=8.4 Hz, 1H), 7.55 (d, J=8.0 Hz, 1H), 7.41-7.39 (m, 1H), 7.36-7.32 (m, 1H), 7.23-7.21 (m, 2H), 7.13-7.11 (m, 2H), 6.90-6.84 (m, 4H), 4.32 (s, 2H), 4.09 (brs, 2H), 2.76 (s, 3H), 1.42 (s, 9H).

### Step 3: Preparation of tert-butyl (4-(4-((4-chloro-2-oxo-2,3-dihydro-1H-imidazo[4,5-c]quinolin-1-yl)methyl)phenoxy)benzyl)(methyl)carbamate

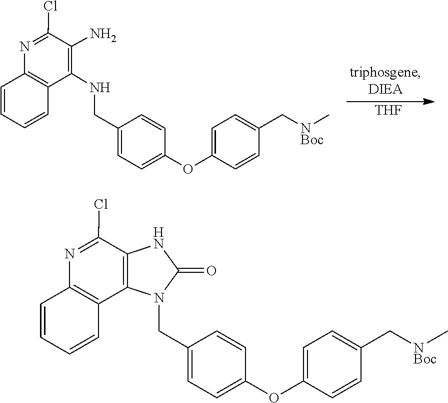 [SMILES: CN(Cc1ccc(Oc2ccc(CNc3c(N)c(Cl)nc4ccccc34)cc2)cc1)C(=O)OC(C)(C)C ; CN(Cc1ccc(Oc2ccc(Cn3c(=O)[nH]c4c(Cl)nc5ccccc5c43)cc2)cc1)C(=O)OC(C)(C)C ; "THF"]

The starting material tert-butyl (4-(4-(((3-amino-2-chloroquinolin-4-yl)amino)methyl)phenoxy)benzyl)(methyl)carbamate (1.07 g, 2.06 mmol, 1.0 eq) was dissolved in anhydrous tetrahydrofuran (10 mL), and triphosgene (611 mg, 2.06 mmol, 1.0 eq) and N,N-diisopropylethylamine (799 mg, 6.18 mmol, 3.0 eq) were added. The reaction mixture was stirred at 25° C. for 18 h. After the reaction was completed, as detected by LCM, the reaction system was concentrated to give a crude product, which was slurried with ethyl acetate (10 mL) for 15 min and filtered, and the filter cake was dried to give the target compound (546 mg, 48.6%). LC-MS: [M+H]⁺=545.03; ¹H NMR (400 MHz, DMSO-d₆): δ 8.03 (d, J=8.4 Hz, 1H), 7.92 (d, J=8.0 Hz, 1H), 7.64-7.60 (m, 1H), 7.52-7.49 (m, 1H), 7.30-7.28 (m, 2H), 7.21-7.19 (m, 2H), 6.96-6.93 (m, 4H), 5.53 (s, 2H), 4.31 (s, 2H), 2.72 (s, 3H), 1.39 (s, 9H).

### Step 4: Preparation of tert-butyl (4-(4-((4-amino-2-oxo-2,3-dihydro-1H-imidazo[4,5-c]quinolin-1-yl)methyl)phenoxy)benzyl)(methyl)carbamate

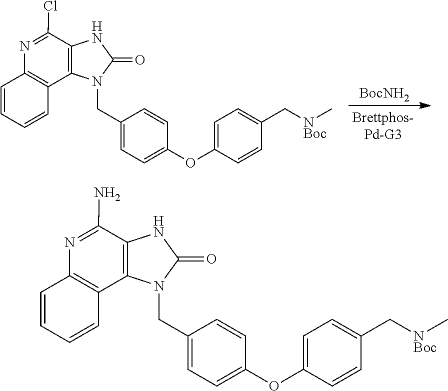 [SMILES: CN(Cc1ccc(Oc2ccc(Cn3c(=O)[nH]c4c(Cl)nc5ccccc5c43)cc2)cc1)C(=O)OC(C)(C)C ; "BocNH2" ; CN(Cc1ccc(Oc2ccc(Cn3c(=O)[nH]c4c(N)nc5ccccc5c43)cc2)cc1)C(=O)OC(C)(C)C]

The starting material tert-butyl (4-(4-((4-chloro-2-oxo-2,3-dihydro-1H-imidazo[4,5-c]quinolin-1-yl)methyl)phenoxy)benzyl)(methyl)carbamate (300 mg, 550 μmol, 1.0 eq) was dissolved in anhydrous tetrahydrofuran (10 mL), and tert-butyl carbamate (645 mg, 5.50 mmol, 10.0 eq), cesium carbonate (538 mg, 1.65 mmol, 3.0 eq), and BrettPhos Pd G3 (methanesulfonato2-dicyclohexylphosphino-3,6-dimethoxy-2′-4′-6′-tri-i-propyl-1,1′-biphenyl)(2′-amino-1,1′-biphenyl-2-yl)palladium(II)) (49.9 mg, 55.0 μmol, 0.1 eq) were added under nitrogen atmosphere. The reaction mixture was stirred at 100° C. for 5 h under nitrogen atmosphere. After the reaction was completed, as detected by LC-MS, the reaction mixture was concentrated by rotary evaporation to remove the solvent, and water (10 mL) was added. The mixture was extracted with ethyl acetate (10 mL×2). The combined organic phases were dried over anhydrous sodium sulfate, filtered and concentrated to give a solid, which was the target product (210 mg, 72.6%). The product was used directly in the next step. LC-MS: [M+H]⁺=526.16.

### Step 5: Preparation of 4-amino-1-(4-(4-((methyl amino)methyl)phenoxy)benzyl)-1,3-dihydro-2H-imidazo[4,5-c]quinolin-2-one

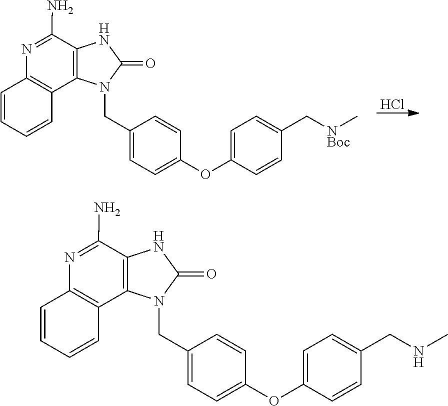 [SMILES: CN(Cc1ccc(Oc2ccc(Cn3c(=O)[nH]c4c(N)nc5ccccc5c43)cc2)cc1)C(=O)OC(C)(C)C ; Cl ; CNCc1ccc(Oc2ccc(Cn3c(=O)[nH]c4c(N)nc5ccccc5c43)cc2)cc1]

The starting material tert-butyl (4-(4-((4-amino-2-oxo-2,3-dihydro-1H-imidazo[4,5-c]quinolin-1-yl)methyl)phenoxy)benzyl)(methyl)carbamate (210 mg, 400 μmol, 1.0 eq) was dissolved in dichloromethane (10 mL), followed by the addition of HCl (4 M in dioxane, 3 mL). The reaction mixture was stirred at 25° C. for 2 h. After the reaction was completed, as detected by LC-MS, the reaction mixture was concentrated to remove the solvent to give a crude product, which was separated and purified by Prep HPLC (0.01% aqueous HCOOH solution, MeCN) to give the target compound (36.7 mg, 21.6%). LC-MS: [M+H]⁺=426.12; ¹H NMR (400 MHz, CD₃OD): δ 8.38 (s, 2H), 7.88 (d, J=8.0 Hz, 1H), 7.65 (d, J=8.0 Hz, 1H), 7.53-7.51 (m, 1H), 7.42-7.40 (m, 2H), 7.32-7.30 (m, 2H), 7.22-7.21 (m, 1H), 7.01-6.99 (m, 4H), 5.57 (s, 2H), 4.12 (s, 2H), 2.69 (s, 3H).

### Example 7: Preparation of 4-amino-1-(3-(pyrrolidin-1-ylmethyl)benzyl)-1H-imidazo[4,5-c]quinoline-2-carboxamide

### Step 1: Preparation of 2-chloro-3-nitro-N-(3-(pyrrolidin-1-ylmethyl)benzyl)quinoline-4-amine

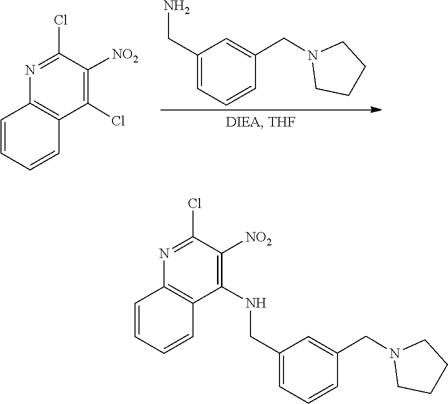 [SMILES: O=[N+]([O-])c1c(Cl)nc2ccccc2c1Cl ; NCc1cccc(CN2CCCC2)c1 ; O=[N+]([O-])c1c(Cl)nc2ccccc2c1NCc1cccc(CN2CCCC2)c1]

The starting material 2,4-dichloro-3-nitroquinoline (3.50 g, 14.4 mmol, 1.0 eq) and (3-(pyrrolidin-1-ylmethyl)phenyl)methylamine (2.74 g, 14.4 mmol, 1.0 eq) were dissolved in tetrahydrofuran (40 mL), and DIEA (2.42 g, 18.7 mmol, 1.3 eq) was added. The reaction mixture was stirred at 25° C. for 16 h. After the reaction was completed, as detected by TLC, the reaction mixture was added to water (50 mL). The mixture was extracted with ethyl acetate (50 mL×2). The combined organic phases were washed with saturated brine, then dried over anhydrous sodium sulfate, filtered and concentrated to give a crude product, which was separated and purified by flash chromatography (silica gel, PE:EA=100:0 to 91:9) to give the target compound (5.00 g, 78.7%). LC-MS: [M+H]⁺=397.72.

### Step 2: Preparation of 2-chloro-N⁴-(3-(pyrrolidin-1-ylmethyl)benzyl)quinoline-3,4-diamine

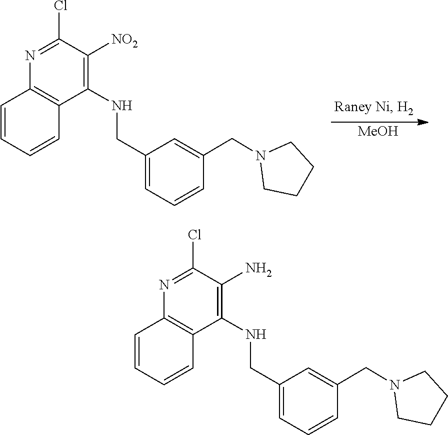

The starting material 2-chloro-3-nitro-N-(3-(pyrrolidin-1-ylmethyl)benzyl)quinolin-4-amine (5.00 g, 12.6 mmol, 1.0 eq) was dissolved in methanol (50 mL), and Raney nickel (1.08 g) was added under nitrogen atmosphere. The reaction mixture was purged with nitrogen 3 times and then purged with hydrogen 3 times. The reaction mixture was stirred at 25° C. for 2 h under hydrogen atmosphere (15 Psi). After the reaction was completed, as detected by TLC, the mixture was filtered through celite and then concentrated to give the target compound (2.10 g, 40.9%). LC-MS: [M+H]⁺=367.07.

### Step 3: Preparation of ethyl 4-chloro-1-(3-(pyrrolidin-1-ylmethyl)benzyl)-1H-imidazo[4,5-c]quinoline-2-carboxylate

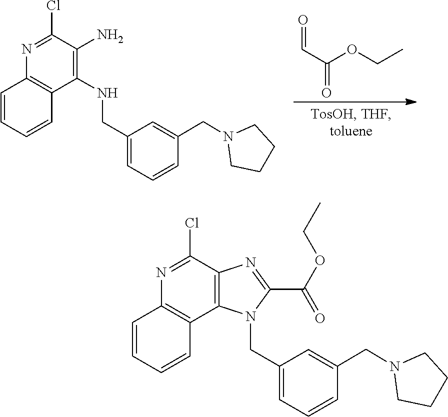

The starting material 2-chloro-N⁴-(3-(pyrrolidin-1-ylmethyl)benzyl)quinoline-3,4-diamine (480 mg, 1.31 mmol, 1.0 eq) was dissolved in toluene (9 mL) and tetrahydrofuran (3 mL), and a solution of ethyl glyoxylate in toluene (50%, 534 mg, 2.62 mmol, 2.0 eq) and p-toluenesulfonic acid (249 mg, 1.44 mmol, 1.1 eq) were added. The mixture was stirred at 100° C. for 1 h. After the reaction was completed, as detected by LC-MS, water (10 mL) was added to the reaction mixture for dilution. The mixture was extracted with ethyl acetate (15 mL×3). The combined organic phases were washed with saturated brine, then dried over anhydrous sodium sulfate, filtered and concentrated to give a crude product, which was separated and purified by flash chromatography (silica gel, DCM:MeOH=100:0 to 94:6) to give the target compound (430 mg, 72.2%). LC-MS: [M+H]⁺=449.07; ¹H NMR (400 MHz, DMSO-d₆): δ 8.32 (s, 1H), 8.16 (d, J=8.4 Hz, 1H), 8.11 (d, J=8.4 Hz, 1H), 7.79-7.75 (m, 1H), 7.59-7.55 (m, 1H), 7.27-7.23 (m, 1H), 7.16 (d, J=7.6 Hz, 1H), 7.08 (s, 1H), 6.97 (d, J=7.6 Hz, 1H), 6.36 (s, 2H), 4.46-4.40 (m, 2H), 3.48 (s, 2H), 2.25 (s, 4H), 1.56 (s, 4H), 1.35 (t, J=7.2 Hz, 1H).

### Step 4: Preparation of ethyl 4-amino-1-(3-(pyrrolidin-1-ylmethyl)benzyl)-1H-imidazo[4,5-c]quinoline-2-carboxylate

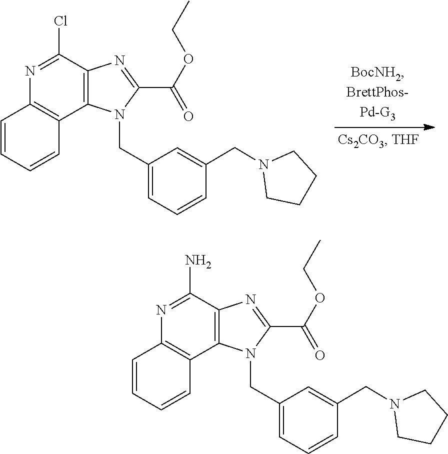

The starting material ethyl 4-chloro-1-(3-(pyrrolidin-1-ylmethyl)benzyl)-1H-imidazo[4,5-c]quinoline-2-carboxylate (1.06 g, 2.36 mmol, 1.0 eq) was dissolved in tetrahydrofuran (12 mL), followed by the addition of tert-butyl carbamate (2.77 g, 23.6 mmol, 10.0 eq), cesium carbonate (2.31 g, 7.08 mmol, 3.0 eq) and BrettPhos Pd G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (214 mg, 236 μcool, 0.1 eq). The reaction mixture was stirred at 100° C. for 4 h under nitrogen atmosphere. After the reaction was completed, as detected by LC-MS, water (10 mL) was added to the reaction system for dilution, and the mixture was extracted with ethyl acetate (15 mL×2). The combined organic phases were washed with saturated brine, then dried over anhydrous sodium sulfate, filtered and concentrated to give a crude product, which was separated and purified by flash chromatography (silica gel, DCM:MeOH=100:0 to 91:9) to give the target compound (220 mg, 21.7%). LC-MS: [M+H]⁺=430.09.

### Step 5: Preparation of 4-amino-1-(3-(pyrrolidin-1-ylmethyl)benzyl)-1H-imidazo[4,5-c]quinoline-2-carboxamide

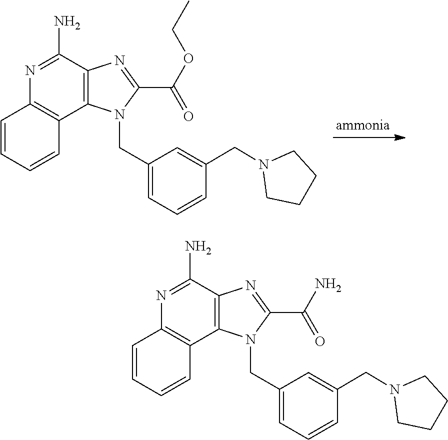

Ethyl 4-amino-1-(3-(pyrrolidin-1-ylmethyl)benzyl)-1H-imidazo[4,5-c]quinoline-2-carboxylate (200 mg, 466 μcool, 1.0 eq) was dissolved in a solution of amine/methanol solution (2 mL). The mixture was stirred at 25° C. for 16 h. After the reaction was completed, as detected by LC-MS, the reaction mixture was filtered, and then the filter cake was dried to give a crude product (140 mg, 75.1%) in the form of a white solid. 120 mg of the crude product was used directly in the next step without purification, and 20 mg of the crude product was separated and purified by Prep-HPLC (0.01% aqueous HCOOH solution, MeCN) to give the target compound (6.5 mg, 3.49%). LC-MS: [M+H]⁺=401.09; ¹H NMR (400 MHz, methanol-d₄): δ 7.99 (d, J=8.0 Hz, 1H), 7.79 (d, J=8.0 Hz, 1H), 7.68 (t, J=8.0 Hz, 1H), 7.49 (t, J=7.6 Hz, 1H), 7.44-7.37 (m, 2H), 7.32-7.30 (d, J=8.0 Hz, 1H), 7.27 (s, 1H), 6.53 (s, 2H), 4.28 (s, 2H), 3.35-3.04 (m, 4H), 2.09-1.91 (m, 4H).

### Example 8: Preparation of 4-amino-1-(3-(pyrrolidin-1-ylmethyl)benzyl)-1H-imidazo[4,5-c]quinoline-2-carbonitrile

### Step 1: Preparation of 4-amino-1-(3-(pyrrolidin-1-ylmethyl)benzyl)-1H-imidazo[4,5-c]quinoline-2-carbonitrile

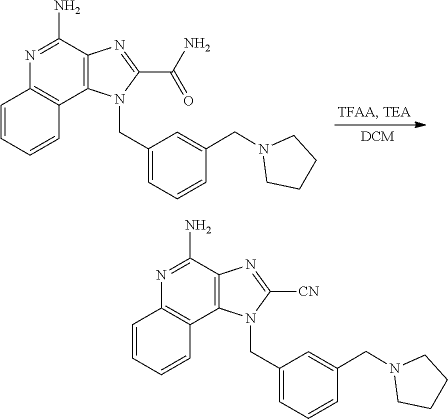

The starting material 4-amino-1-(3-(pyrrolidin-1-ylmethyl)benzyl)-1H-imidazo[4,5-c]quinoline-2-carboxamide (100 mg, 250 μmol, 1.0 eq) was dissolved in dichloromethane (3 mL), followed by the addition of triethylamine (50.5 mg, 499 μcool, 69.5 μL, 2.0 eq). Trifluoroacetic anhydride (78.7 mg, 375 μmol, 52.1 mL, 1.5 eq) was slowly added dropwise to the reaction mixture at 0° C. The reaction mixture was stirred at 0° C. for 1.5 h and then at 25° C. for 14.5 h. After the starting material was consumed completely, as detected by LC-MS, the reaction mixture was concentrated to give a crude product, which was separated and purified by Prep-HPLC (0.01% aqueous HCOOH solution, MeCN) to give the target compound (31.0 mg, 32.5%). LC-MS: [M+H]⁺=383.09; H NMR (400 MHz, DMSO-d₆): δ 7.91-7.89 (d, J=8.0 Hz, 1H), 7.59-7.57 (d, J=7.2 Hz, 1H), 7.46 (t, J=7.2 Hz, 1H), 7.28 (t, J=7.6 Hz, 1H), 7.20 (t, J=7.6 Hz, 1H), 7.15-7.10 (m, 4H), 7.01 (d, J=7.6 Hz, 1H), 6.04 (s, 2H), 3.50 (s, 2H), 2.28 (s, 4H), 1.58 (s, 4H).

### Example 9: Preparation of 4-amino-1-(3-((2-oxopyrrolidin-1-yl)methyl)benzyl)-1H-imidazo[4,5-c]quinolin-2(3H)-one

### Step 1: Preparation of 3-((2-oxopyrrolidin-1-yl)methyl)benzonitrile

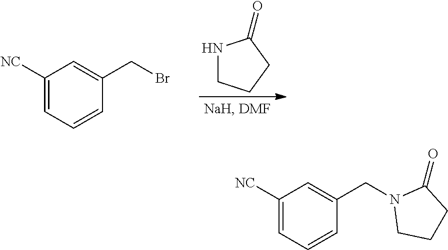 [SMILES: N#Cc1cccc(CBr)c1 ; O=C1CCCN1 ; N#Cc1cccc(CN2CCCC2=O)c1]

The starting material pyrrolidin-2-one (2.16 g, 25.4 mmol, 1.0 eq) was dissolved in tetrahydrofuran (45 mL) and N,N-dimethylformamide (6 mL), and sodium hydride (1.12 g, 28.1 mmol, 60% purity, 1.1 eq) was added at 0° C. under nitrogen atmosphere. The mixture was stirred for 30 min, and then 3-(bromomethyl)benzonitrile (5.00 g, 25.5 mmol, 1.0 eq) was added. The reaction mixture was stirred at 50° C. for 15.5 h. After the reaction was completed, as detected by TLC (PE:EA=1:1), water (60 mL) was added to the reaction mixture. The mixture was extracted with ethyl acetate (20 mL×3). The combined organic phases were washed with saturated brine, dried over anhydrous sodium sulfate, filtered and concentrated to give a crude product, which was separated and purified by flash chromatography (PE:EA=100:0 to 1:1) to give the target compound (2.90 g, 51.1%). LC-MS: [M+H]⁺=201.07.

### Step 2: Preparation of 1-(3-(aminomethyl)benzyl)pyrrolidin-2-one

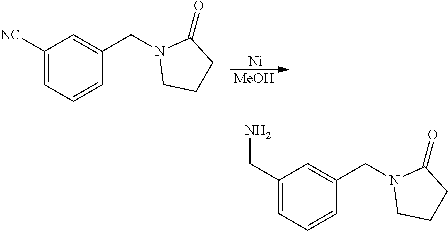

The starting material 3-((2-oxopyrrolidin-1-yl)methyl)benzonitrile (2.80 g, 14.0 mmol, 1.0 eq) was dissolved in methanol (25 mL), and Raney nickel (1.20 g) was added. Then the reaction mixture was purged with nitrogen 3 times and then purged with hydrogen 3 times. The reaction mixture was stirred at 25° C. for 4 h under hydrogen atmosphere (15 Psi). After the reaction was completed, as detected by LC-MS, the mixture was filtered. The filter cake was washed with methanol, and the filtrate was concentrated to remove methanol to give the target compound (2.60 g, 91.0%), which was used directly in the next step. LC-MS: [M+H]⁺=205.08; ¹H NMR (400 MHz, CDCl₃): δ 7.32-7.11 (m, 4H), 4.45 (s, 2H), 3.86 (s, 2H), 3.29-3.25 (m, 2H), 2.47-2.43 (m, 2H), 2.03-1.96 (m, 2H).

### Step 3: Preparation of 1-(3-(((2-chloro-3-nitroquinolin-4-yl)amino)methyl)benzyl)pyrrolidin-2-one

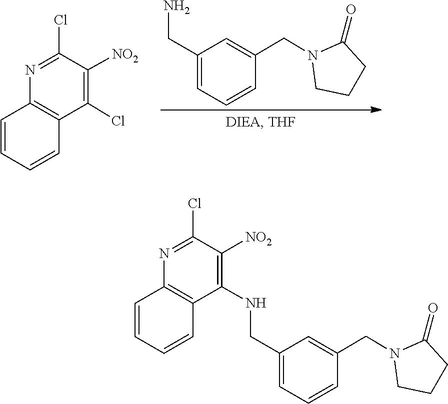 [SMILES: O=[N+]([O-])c1c(Cl)nc2ccccc2c1Cl ; NCc1cccc(CN2CCCC2=O)c1 ; O=C1CCCN1Cc1cccc(CNc2c([N+](=O)[O-])c(Cl)nc3ccccc23)c1]

The starting material 1-(3-(aminomethyl)benzyl)pyrrolidin-2-one (2.00 g, 9.79 mmol, 1.0 eq) was dissolved in tetrahydrofuran (20 mL), N,N-diisopropylethylamine (2.53 g, 19.6 mmol, 2 eq) was added, and then 2,4-dichloro-3-nitroquinoline (2.38 g, 9.79 mmol, 1 eq) was added. The mixture was stirred at 25° C. for 14 h. After the reaction was completed, as detected by LC-MS, the reaction mixture was filtered, the filter cake was washed with tetrahydrofuran, and the filtrate was concentrated to remove tetrahydrofuran. Water (100 mL) was added. The mixture was extracted with dichloromethane (150 mL×3). The organic phases were washed with saturated brine (100 mL), and the combined organic phases were dried over anhydrous sodium sulfate, then filtered and concentrated to give a crude product, which was separated and purified by flash chromatography (DCM:MeOH=100:0 to 10:1) to give the target compound (3.10 g, 77.1%). LC-MS: [M+H]⁺=411.11.

### Step 4: Preparation of 1-(3-(((3-amino-2-chloroquinolin-4-yl)amino)methyl)benzyl)pyrrolidin-2-one

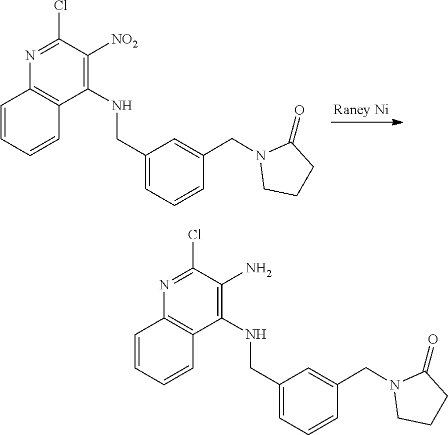

1-(3-(((2-Chloro-3-nitroquinolin-4-yl)amino)methyl)benzyl)pyrrolidin-2-one (2.00 g, 4.87 mmol, 1.0 eq) was dissolved in methanol (30 mL), and Raney nickel (1.25 g) was added. The reaction mixture was purged with nitrogen 3 times and then purged with hydrogen 3 times. The reaction mixture was stirred at 25° C. for 4 h under hydrogen atmosphere (15 Psi). After the reaction was completed, as detected by LC-MS, the mixture was filtered. The filter cake was washed with methanol and concentrated to remove methanol to give a crude product, which was separated and purified by flash chromatography (DCM:MeOH=100:0 to 10:1) to give the target compound (1.2 g, 64.72%). LC-MS: [M+H]⁺=381.07.

### Step 5: Preparation of 4-chloro-1-(3-((2-oxopyrrolidin-1-yl)methyl)benzyl)-1H-imidazo[4,5-c]quinolin-2(3H)-one

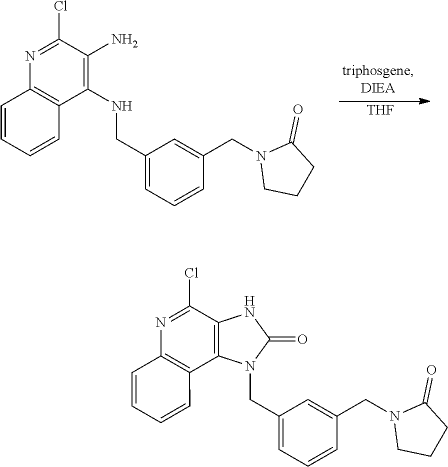 [SMILES: Nc1c(Cl)nc2ccccc2c1NCc1cccc(CN2CCCC2=O)c1 ; O=C1CCCN1Cc1cccc(Cn2c(=O)[nH]c3c(Cl)nc4ccccc4c32)c1 ; "THF"]

1-(3-(((3-Amino-2-chloroquinolin-4-yl)amino)methyl)benzyl)pyrrolidin-2-one (1.00 g, 2.63 mmol, 1.0 eq) was dissolved in tetrahydrofuran (10 mL), N,N-diisopropylethylamine (1.02 g, 7.88 mmol, 3.0 eq) was added, and then triphosgene (779 mg, 2.63 mmol, 1.0 eq) was added The reaction mixture was stirred at 25° C. for 14 h. After the reaction was completed, as detected by TLC (DCM:MeOH=20:1, 254 nm), the reaction mixture was filtered, the filter cake was washed with tetrahydrofuran, and the filtrate was concentrated. Then water (20 mL) was added, and the mixture was extracted with dichloromethane (20 mL×3). The combined organic phases were washed with saturated brine (20 mL), dried over anhydrous sodium sulfate, filtered and concentrated to give a crude product, which was separated and purified by flash chromatography (DCM:MeOH=100:0 to 3:1) to give the target compound (800 mg, 74.9%). LC-MS: [M+H]⁺=407.02.

### Step 6: Preparation of 4-amino-1-(3-((2-oxopyrrolidin-1-yl)methyl)benzyl)-1H-imidazo[4,5-c]quinolin-2(3H)-one

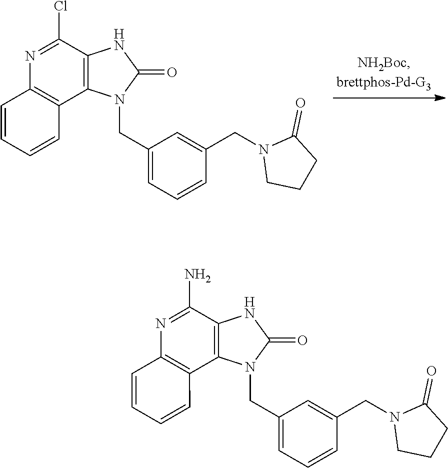 [SMILES: O=C1CCCN1Cc1cccc(Cn2c(=O)[nH]c3c(Cl)nc4ccccc4c32)c1 ; Nc1nc2ccccc2c2c1[nH]c(=O)n2Cc1cccc(CN2CCCC2=O)c1]

The starting materials 4-chloro-1-(3-((2-oxopyrrolidin-1-yl)methyl)benzyl)-1H-imidazo[4,5-c]quinolin-2(3H)-one (200 mg, 492 μmol, 1.0 eq) and tert-butyl carbamate (576 mg, 4.92 mmol, 10.0 eq) were dissolved in tetrahydrofuran (10 mL), and cesium carbonate (480 mg, 1.47 mmol, 3.0 eq) and BrettPhos Pd G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (44.6 mg, 49.2 μmol, 0.1 eq) were added under nitrogen atmosphere. The reaction mixture was stirred at 100° C. for 2 h. After the reaction was completed, as detected by LC-MS, the reaction mixture was filtered. The filter cake was washed with tetrahydrofuran. The filtrate was concentrated to give a crude product, which was separated and purified by Prep-HPLC (0.225% aqueous formic acid solution, MeCN) to give the target compound (20.0 mg, 10.5%). LC-MS: [M+H]⁺=388.30; ¹H NMR (400 MHz, CDCl₃): δ 7.71-7.70 (m, 1H), 7.58 (d, J=7.2 Hz, 1H), 7.39-7.24 (m, 2H), 7.12-7.09 (m, 3H), 6.91 (s, 1H), 5.50 (s, 2H), 4.35 (s, 2H), 2.95 (t, J=7.0 Hz, 2H), 2.23-2.21 (m, 2H), 1.72-1.70 (m, 2H).

### Example 10: Preparation of 4-amino-1-(4-(pyrrolidin-1-ylmethyl)benzyl)-1H-imidazo[4,5-c]quinolin-2(3H)-one

### Step 1: Preparation of 4-(pyrrolidin-1-ylmethyl)benzonitrile

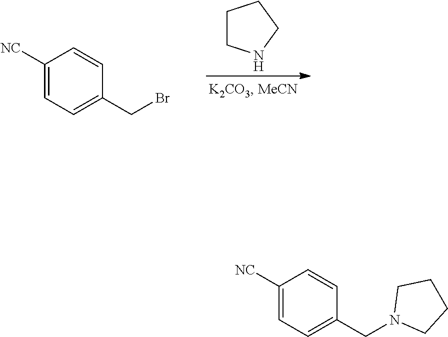 [SMILES: N#Cc1ccc(CBr)cc1 ; C1CCNC1 ; N#Cc1ccc(CN2CCCC2)cc1]

The starting material 4-(bromomethyl)benzonitrile (5.00 g, 25.5 mmol, 1.0 eq) was dissolved in acetonitrile (50 mL), and pyrrolidine (2.18 g, 30.6 mmol, 2.55 mL, 1.2 eq) and potassium carbonate (10.6 g, 76.5 mmol, 3.0 eq) were added. The reaction mixture was stirred at 25° C. for 1 h. After the reaction was completed, as detected by TLC (PE:EA=10:1), the reaction mixture was concentrated to remove acetonitrile to give a crude product. Then water (20 mL) was added. The mixture was extracted with ethyl acetate (20 mL×3). The combined organic phases were dried over anhydrous sodium sulfate, then filtered and concentrated to give the target compound (5.00 g, crude) in the form of a light yellow oil, which was used directly in the next step. ¹H NMR (400 MHz, CD₃OD): δ 7.70-7.55 (m, 4H), 3.71-3.70 (m, 2H), 2.55 (br s, 4H) 1.82 (br s, 4H).

### Step 2: Preparation of (4-(pyrrolidin-1-ylmethyl)phenyl)methylamine

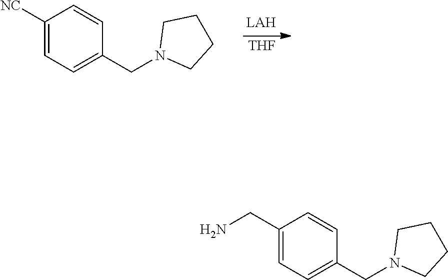 [SMILES: N#Cc1ccc(CN2CCCC2)cc1 ; NCc1ccc(CN2CCCC2)cc1 ; "THF"]

The starting material 4-(pyrrolidin-1-ylmethyl)benzonitrile (5.00 g, 26.9 mmol, 1.0 eq) was dissolved in tetrahydrofuran (50 mL), and lithium aluminum hydride (2.00 g, 52.7 mmol, 2.0 eq) was added slowly at 0° C. The reaction mixture was stirred at 25° C. for 2 h. After the reaction was completed, as detected by TLC (DCM:MeOH=10:1, UV), water (2 mL), 15% aqueous sodium hydroxide solution (2 mL), and water (6 mL) were added to the reaction mixture slowly and successively at 0° C. The mixture was stirred for 30 min and extracted with ethyl acetate (10 mL×3). The organic phases were washed with saturated brine (20 mL), dried over anhydrous sodium sulfate, filtered and concentrated to give the target compound (4.10 g, 80.3%), which was used directly in the next step.

### Step 3: Preparation of 2-chloro-3-nitro-N-(4-(pyrrolidin-1-ylmethyl)benzyl)quinoline-4-amine

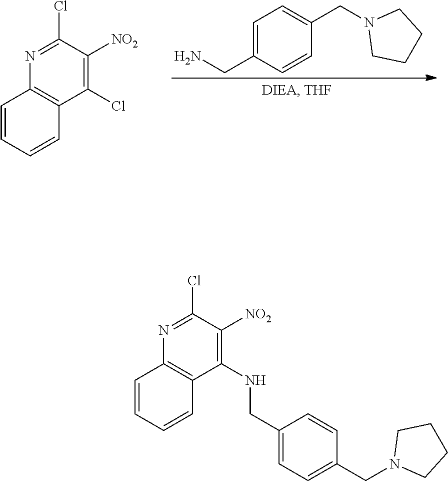 [SMILES: O=[N+]([O-])c1c(Cl)nc2ccccc2c1Cl ; NCc1ccc(CN2CCCC2)cc1 ; O=[N+]([O-])c1c(Cl)nc2ccccc2c1NCc1ccc(CN2CCCC2)cc1]

The starting material 2,4-dichloro-3-nitroquinoline (1.00 g, 4.11 mmol, 1.0 eq) was dissolved in tetrahydrofuran (15 mL), and (4-(pyrrolidin-1-ylmethyl)phenyl)methylamine (940 mg, 4.94 mmol, 1.2 eq) and N,N-diisopropylethylamine (1.60 g, 12.3 mmol, 3.0 eq) were added. The reaction mixture was stirred at 25° C. for 12 h. After the reaction was completed, as detected by LC-MS, the reaction mixture was concentrated to remove tetrahydrofuran, and then water (10 mL) was added. The mixture was extracted with dichloromethane (10 mL×3). The combined organic phases were dried over anhydrous sodium sulfate, filtered and concentrated to give a crude product, which was separated and purified by flash chromatography (DCM:MeOH=100:0 to 97:3) to give the target compound (1.40 g, 85.7%). LC-MS: [M+H]⁺=397.21.

### Step 4: Preparation of 2-chloro-N⁴-(4-(pyrrolidin-1-ylmethyl)benzyl)quinoline-3,4-diamine

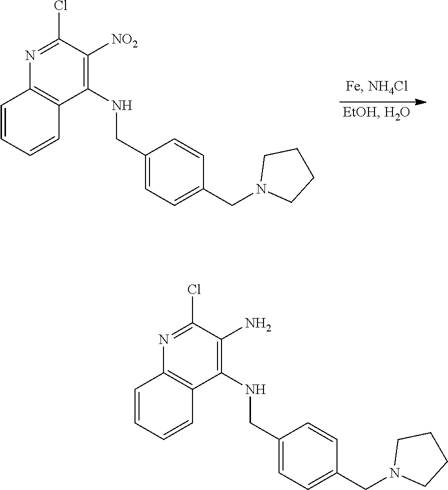

The starting material 2-chloro-3-nitro-N-(4-(pyrrolidin-1-ylmethyl)benzyl)quinolin-4-amine (1.00 g, 2.52 mmol, 1.0 eq) was dissolved in ethanol (11 mL) and water (2 mL), and iron powder (563 mg, 10.1 mmol, 4.0 eq) and ammonium chloride (539 mg, 10.1 mmol, 4.0 eq) were added at 25° C. The reaction mixture was stirred at 100° C. for 1 h. After the reaction was completed, as detected by LC-MS, the reaction mixture was filtered. The filter cake was washed with ethanol. The filtrate was concentrated to give a black crude product. The crude product was separated and purified by flash chromatography (DCM:MeOH=98:2 to 93:7) to give the target compound (510 mg, 55.2%). LC-MS: [M+H]⁺=367.01.

### Step 5: Preparation of 4-chloro-1-(4-(pyrrolidin-1-ylmethyl)benzyl)-1H-imidazo[4,5-c]quinolin-2(3H)-one

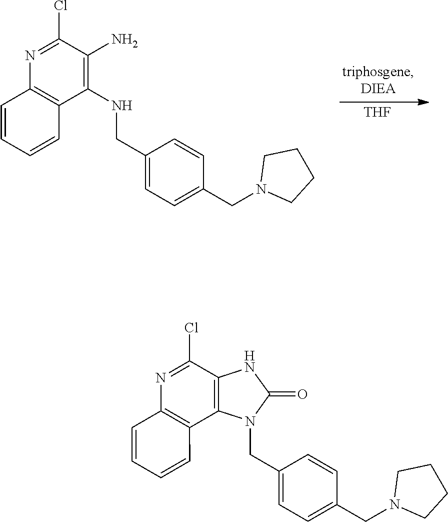 [SMILES: Nc1c(Cl)nc2ccccc2c1NCc1ccc(CN2CCCC2)cc1 ; O=c1[nH]c2c(Cl)nc3ccccc3c2n1Cc1ccc(CN2CCCC2)cc1 ; "THF"]

The starting material 2-chloro-N⁴-(4-(pyrrolidin-1-ylmethyl)benzyl)quinoline-3,4-diamine (510 mg, 1.39 mmol, 1.0 eq) was dissolved in tetrahydrofuran (5 mL), N,N-diisopropylethylamine (539 mg, 4.17 mmol, 3.0 eq) was added, and then triphosgene (470 mg, 1.58 mmol, 1.1 eq) was added. The reaction mixture was stirred at 25° C. for 15 h. After the reaction was completed, as detected by LC-MS, the reaction mixture was concentrated to give a crude product, which was separated and purified by flash chromatography (DCM:MeOH=97:3 to 96:4) to give the target compound (320 mg, 58.6%). LC-MS: [M+H]⁺=392.91.

### Step 6: Preparation of 4-amino-1-(4-(pyrrolidin-1-ylmethyl)benzyl)-1H-imidazo[4,5-c]quinolin-2(3H)-one

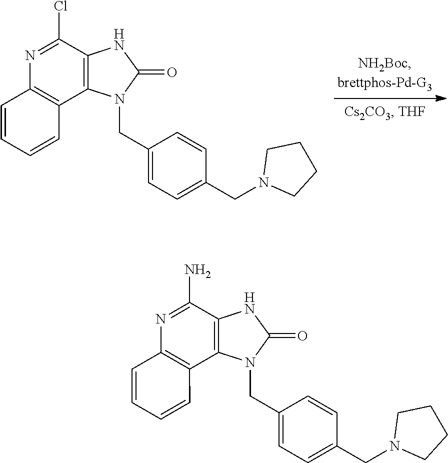 [SMILES: O=c1[nH]c2c(Cl)nc3ccccc3c2n1Cc1ccc(CN2CCCC2)cc1 ; Nc1nc2ccccc2c2c1[nH]c(=O)n2Cc1ccc(CN2CCCC2)cc1]

The starting material 4-chloro-1-(4-(pyrrolidin-1-ylmethyl)benzyl)-1H-imidazo[4,5-c]quinolin-2(3H)-one (100 mg, 255 μmol, 1 eq) was dissolved in tetrahydrofuran (2 mL), and tert-butyl carbamate (298 mg, 2.55 mmol, 10 eq), cesium carbonate (249 mg, 764 μmol, 3.0 eq), and BrettPhos-Pd-G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (23.1 mg, 25.5 μmol, 0.1 eq) were added respectively under nitrogen atmosphere. The reaction mixture was stirred at 100° C. for 15 h. After the reaction was completed, as detected by LC-MS, the reaction mixture was filtered, and the filtrate was concentrated to give a crude product, which was separated and purified by Prep-HPLC (0.225% aqueous HCOOH solution, MeCN) to give the target compound (20.0 mg, 21.0%). LC-MS: [M+H]⁺=374.30; ¹H NMR (400 MHz, DMSO-d₆): δ 11.06 (brs, 1H), 8.18 (s, 1H), 7.73 (d, J=8.4 Hz, 1H), 7.53 (d, J=8.4 Hz, 1H), 7.34-7.32 (m, 1H), 7.28-7.26 (m, 2H), 7.20-7.18 (m, 2H), 7.06-7.04 (m, 1H), 6.38 (s, 2H), 5.45 (s, 2H), 3.58 (s, 2H), 2.45 (brs, 4H), 1.68 (brs, 4H).

### Example 11: Preparation of 4-amino-1-(3-(piperidin-1-ylmethyl)benzyl)-1H-imidazo[4,5-c]quinolin-2(3H)-one

### Step 1: Preparation of 3-(piperidin-1-ylmethyl)benzonitrile

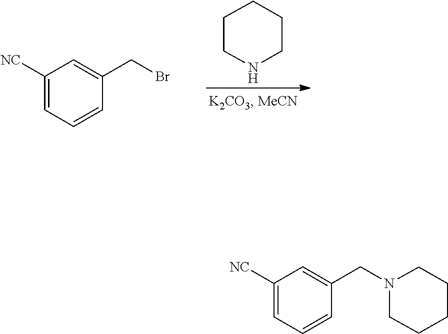 [SMILES: N#Cc1cccc(CBr)c1 ; C1CCNCC1 ; N#Cc1cccc(CN2CCCCC2)c1]

The starting material 3-(bromomethyl)benzonitrile (15.0 g, 76.5 mmol, 1.0 eq) was dissolved in acetonitrile (150 mL), and potassium carbonate (31.7 g, 229 mmol, 3.0 eq) and tetrahydropyrrole (7.17 g, 84.2 mmol, 1.1 eq) were added successively. The reaction mixture was stirred at 25° C. for 1 h. After the reaction was completed, as detected by LC-MS, the reaction mixture was filtered, the filtrate was concentrated, and then water (300 mL) was added. The mixture was extracted with dichloromethane (200 mL×2). The combined organic phases were washed with saturated brine (300 mL), then dried over anhydrous sodium sulfate, filtered and concentrated to give a solid. The solid was separated and purified by flash chromatography (DCM:MeOH=1:0) to give the target compound (12.2 g, 79.6%). LC-MS: [M+H]⁺=201.31.

### Step 2: Preparation of (3-(piperidin-1-ylmethyl)phenyl)methylamine

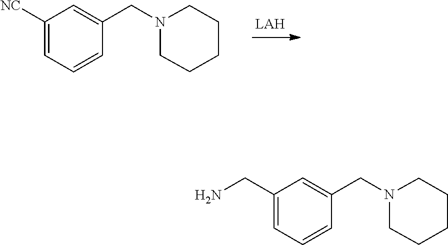 [SMILES: N#Cc1cccc(CN2CCCCC2)c1 ; NCc1cccc(CN2CCCCC2)c1]

3-(Piperidin-1-ylmethyl)benzonitrile (3.00 g, 14.9 mmol, 1.0 eq) was dissolved in tetrahydrofuran (20 mL), and lithium aluminium hydride (1.14 g, 29.9 mmol, 2.0 eq) was added slowly at 0° C. The reaction mixture was stirred at 25° C. for 1 h. After the reaction was completed, as detected by LC-MS, water (1 mL), 15% aqueous NaOH solution (1 mL), and water (3 mL) were successively added to the reaction mixture. Then the mixture was dried over anhydrous sodium sulfate, filtered and concentrated to give the target compound (2.80 g, 91.49%) in the form of a colorless oil. The crude product was used directly in the next step. LC-MS: [M+H]⁺=205.01.

### Step 3: Preparation of 2-chloro-3-nitro-N-(3-(piperidin-1-ylmethyl)benzyl)quinoline-4-amine

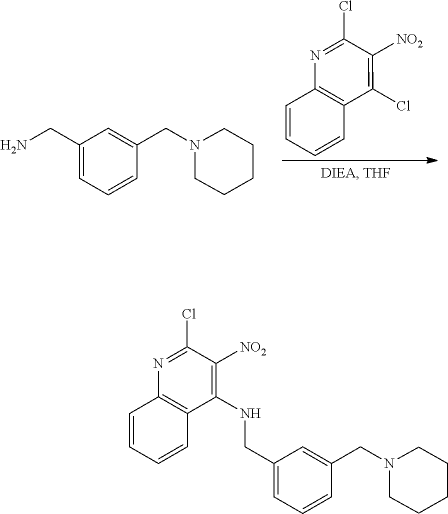

The starting material 2,4-dichloro-3-nitroquinoline (3.17 g, 13.1 mmol, 1.0 eq) was dissolved in anhydrous tetrahydrofuran (40 mL), followed by the addition of N,N-diisopropylethylamine (5.06 g, 39.2 mmol, 3.0 eq) and (3-(piperidin-1-ylmethyl)phenyl)methylamine (2.80 g, 13.7 mmol, 1.1 eq). The reaction mixture was stirred at 25° C. for 18 h. After the reaction was completed, as detected by LC-MS, water (40 mL) was added. The mixture was extracted with ethyl acetate (40 mL×2). The combined organic phases were washed with saturated brine (30 mL×2), then dried over anhydrous sodium sulfate, filtered and concentrated to give a solid. The solid was separated and purified by flash chromatography (DCM:MeOH=100:0 to 93:7) to give the target compound (4.40 g, 82.0%). LC-MS: [M+H]⁺=411.31.

### Step 4: Preparation of 2-chloro-N⁴-(3-(piperidin-1-ylmethyl)benzyl)quinoline-3,4-diamine

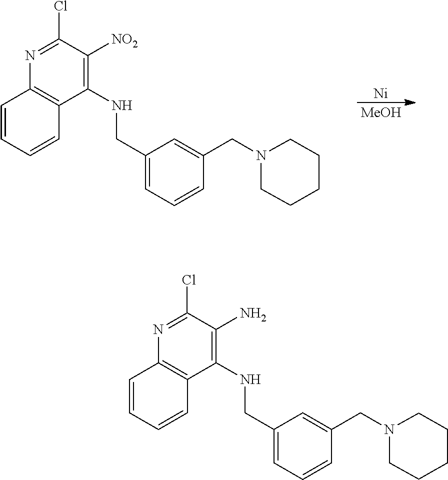

The starting material 2-chloro-3-nitro-N-(3-(piperidin-1-ylmethyl)benzyl)quinolin-4-amine (1.00 g, 2.43 mmol, 1.0 eq) was dissolved in methanol (6 mL), and Raney nickel (208 mg, 2.43 mmol, 1.0 eq) was added. The reaction mixture was purged with nitrogen 3 times and then purged with hydrogen 3 times, and the reaction mixture was stirred at 25° C. for 4 h under hydrogen atmosphere (15 Psi). After the reaction was completed, as detected by LC-MS, the reaction mixture was filtered, the filter cake was washed with methanol (10 mL), and the filtrate was concentrated to give a crude product, which was separated and purified by flash chromatography (DCM:MeOH=100:0 to 91:9) to give the target compound (1.10 g, 59.3%). LC-MS: [M+H]⁺=381.31.

### Step 5: Preparation of 4-chloro-1-(3-(piperidin-1-ylmethyl)benzyl)-1H-imidazo[4,5-c]quinolin-2(3H)-one

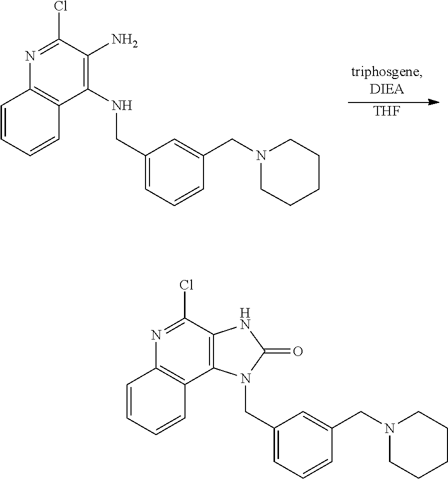 [SMILES: Nc1c(Cl)nc2ccccc2c1NCc1cccc(CN2CCCCC2)c1 ; O=c1[nH]c2c(Cl)nc3ccccc3c2n1Cc1cccc(CN2CCCCC2)c1 ; "THF"]

The starting material 2-chloro-N⁴-(3-(piperidin-1-ylmethyl)benzyl)quinoline-3,4-diamine (1.10 g, 2.89 mmol, 1.0 eq) was dissolved in anhydrous tetrahydrofuran (10 mL), followed by the addition of N,N-diisopropylethylamine (1.12 g, 8.66 mmol, 3.0 eq) and triphosgene (856 mg, 2.89 mmol, 1.0 eq). The reaction mixture was stirred at 25° C. for 8 h. After the reaction was completed, as detected by LC-MS, the reaction mixture was concentrated and slurried with ethyl acetate (8 mL) to give the target product (400 mg, 34.0%). LC-MS: [M+H]⁺=407.31.

### Step 6: Preparation of 4-amino-1-(3-(piperidin-1-ylmethyl)benzyl)-1H-imidazo[4,5-c]quinolin-2(3H)-one

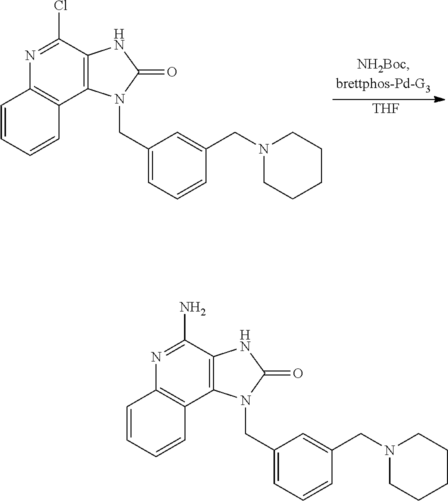 [SMILES: O=c1[nH]c2c(Cl)nc3ccccc3c2n1Cc1cccc(CN2CCCCC2)c1 ; Nc1nc2ccccc2c2c1[nH]c(=O)n2Cc1cccc(CN2CCCCC2)c1 ; "THF"]

The starting material 4-chloro-1-(3-(piperidin-1-ylmethyl)benzyl)-1H-imidazo[4,5-c]quinolin-2(3H)-one (400 mg, 983 μmol, 1.0 eq) was dissolved in tetrahydrofuran (5 mL), and tert-butyl carbamate (1.15 g, 9.83 mmol, 10.0 eq), cesium carbonate (960 mg, 2.95 mmol, 3.0 eq), and brettphos-Pd-G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (89.1 mg, 98.3 μmol, 0.1 eq) were added successively under nitrogen atmosphere. The reaction mixture was stirred at 25° C. for 8 h. After the reaction was completed, as detected by LC-MS, water (10 mL) was added. The mixture was extracted with ethyl acetate (10 mL×2). The combined organic phases were washed with saturated brine (10 mL×2), then dried over anhydrous sodium sulfate, filtered and concentrated to give a crude product, which was separated and purified by Prep-HPLC (0.01% aqueous HCCOH solution, MeCN) to give the target compound (2.12 mg, 0.53%). LC-MS: [M+H]⁺=388.41; ¹H NMR (400 MHz, MeOH-d₄): δ 7.73 (d, J=8.4 Hz, 1H), 7.60 (d, J=8.4 Hz, 1H), 7.40-7.35 (m, 2H), 7.33-7.21 (m, 2H), 7.13 (s, 1H), 7.07-7.05 (m, 1H), 5.59 (s, 2H), 3.57-3.54 (m, 2H), 2.28 (brs, 4H), 1.43 (brs, 4H), 1.34-1.29 (m, 2H).

### Example 12: Preparation of 1-(3-((1H-imidazol-1-yl)methyl)benzyl)-4-amino-1H-imidazo[4,5-c]quinolin-2(3H)-one

### Step 1: Preparation of 3-((1H-imidazol-1-yl)methyl)benzonitrile

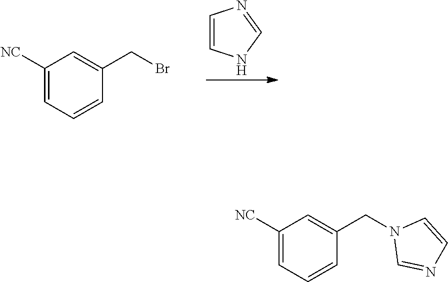 [SMILES: N#Cc1cccc(CBr)c1 ; c1c[nH]cn1 ; N#Cc1cccc(Cn2ccnc2)c1]

The starting materials 3-(bromomethyl)benzonitrile (14.0 g, 71.4 mmol, 1.0 eq) and 1H-imidazole (5.35 g, 78.6 mmol, 1.1 eq) were dissolved in acetonitrile (140 mL), and potassium carbonate (29.6 g, 214 mmol, 3.0 eq) was added at room temperature. Then the reaction mixture was warmed to 80° C. and stirred for 1 h. After the reaction was completed, as detected by LC-MS, the reaction mixture was cooled to room temperature and filtered, and the filter cake was washed with ethyl acetate. The filtrate was concentrated to remove acetonitrile, and then water (200 mL) was added. The mixture was extracted with ethyl acetate (200 mL×2), and the combined organic phases were dried over anhydrous sodium sulfate, filtered and concentrated to give a crude product, which was separated and purified by flash chromatography (DCM:MeOH=100:0 to 20:1) to give the target compound (5.20 g, 39.7%). LC-MS: [M+H]⁺=184.31.

### Step 2: Preparation of (3-((1H-imidazol-1-yl)methyl)phenyl)methylamine

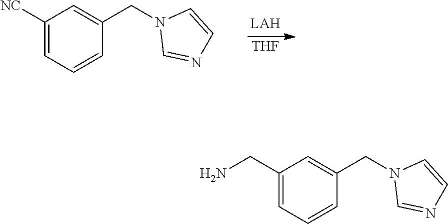 [SMILES: N#Cc1cccc(Cn2ccnc2)c1 ; NCc1cccc(Cn2ccnc2)c1 ; "THF"]

Lithium aluminum hydride (1.04 g, 27.3 mmol, 2.0 eq) was dissolved in tetrahydrofuran (30 mL), and 3-((1H-imidazol-1-yl)methyl)benzonitrile (2.50 g, 13.7 mmol, 1.0 eq) was added at 0° C. The reaction mixture was warmed to 25° C. and stirred for 2 h. After the reaction was completed, as detected by LC-MS, water (1 mL) was added to the reaction mixture at 0° C., then 15% aqueous sodium hydroxide solution (1 mL) was added, and water (3 mL) was finally added. The mixture was stirred for 30 min and then filtered. The filtrate was extracted with ethyl acetate (10 mL×3). The combined organic phases were washed with a saturated sodium chloride solution (20 mL), dried over anhydrous sodium sulfate, filtered and concentrated to give a crude product, which was separated and purified by flash chromatography (DCM:MeOH=100:0 to 10:1) to give the target compound (1.50 g, 8.71%). LC-MS: [M+H]⁺=188.21.

### Step 3: Preparation of N-(3-((1H-imidazol-1-yl)methyl)benzyl)-2-chloro-3-nitroquinoline-4-amine

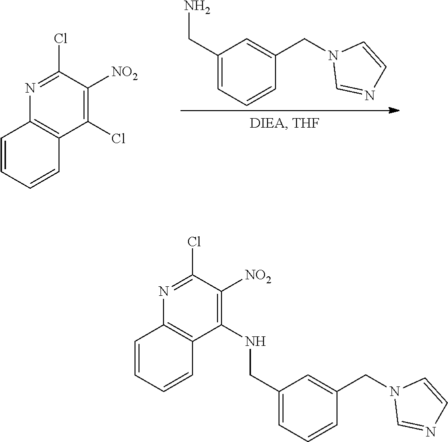 [SMILES: O=[N+]([O-])c1c(Cl)nc2ccccc2c1Cl ; NCc1cccc(Cn2ccnc2)c1 ; O=[N+]([O-])c1c(Cl)nc2ccccc2c1NCc1cccc(Cn2ccnc2)c1]

The starting material 2,4-dichloro-3-nitroquinoline (1.00 g, 4.11 mmol, 1 eq) was dissolved in tetrahydrofuran (15 mL), and (3-((1H-imidazol-1-yl)methyl)phenyl)methylamine (770 mg, 4.11 mmol, 1 eq) and N,N-diisopropylethylamine (1.60 g, 12.3 mmol, 3.0 eq) were added. The reaction mixture was stirred at 25° C. for 4 h. After the reaction was completed, as detected by LC-MS, the reaction mixture was concentrated to remove tetrahydrofuran to give a crude product. Then water (10 mL) was added, and the mixture was extracted with ethyl acetate (20 mL×3). The combined organic phases were dried over anhydrous sodium sulfate, then filtered and concentrated to give a crude product, which was separated and purified by flash chromatography (DCM:MeOH=100:0 to 100:10) to give the target compound (1.30 g, 80.2%). LC-MS: [M+H]⁺=394.20

### Step 4: Preparation of 2-chloro-N⁴-[[3-(imidazol-1-ylmethyl)phenyl]methyl]quinoline-3,4-diamine

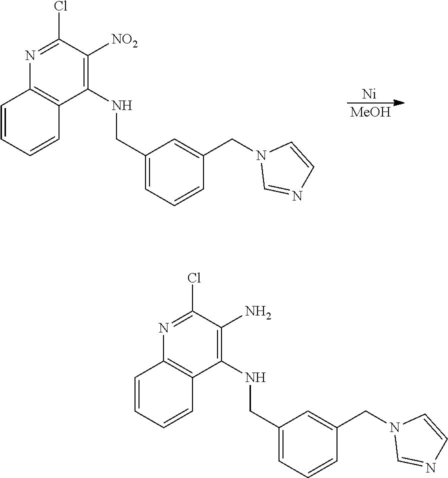

The starting material N-(3-((1H-imidazol-1-yl)methyl)benzyl)-2-chloro-3-nitroquinolin-4-amine (1.20 g, 3.05 mmol, 1 eq) was dissolved in tetrahydrofuran (15 mL), and Raney nickel (261 mg) was added. Then the reaction mixture was purged with nitrogen 3 times, purged with hydrogen 3 times, and stirred at 25° C. for 4 h under hydrogen atmosphere (15 psi). After the reaction was completed, as detected by LC-MS, the reaction mixture was filtered, the filter cake was washed with methanol, and the filtrate was concentrated to give a crude product, which was separated and purified by flash chromatography (DCM:MeOH=100:0 to 10:1) to give the target compound (520 mg, 46.9%). LC-MS: [M+H]⁺=364.10.

### Step 5: Preparation of 1-(3-((1H-imidazol-1-yl)methyl)benzyl)-4-chloro-1H-imidazo[4,5-c]quinolin-2(3H)-one

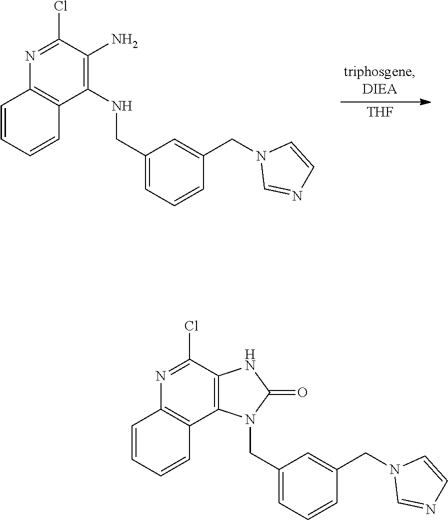 [SMILES: Nc1c(Cl)nc2ccccc2c1NCc1cccc(Cn2ccnc2)c1 ; O=c1[nH]c2c(Cl)nc3ccccc3c2n1Cc1cccc(Cn2ccnc2)c1 ; "THF"]

The starting material 2-chloro-N⁴-[[3-(imidazol-1-ylmethyl)phenyl]methyl]quinoline-3,4-diamine (800 mg, 2.20 mmol, 1 eq) was dissolved in tetrahydrofuran (10 mL), and triphosgene (652 mg, 2.20 mmol, 1 eq) and N,N-diisopropylethylamine (852.52 mg, 6.60 mmol, 3.0 eq) were added. The reaction mixture was stirred at 25° C. for 4 h. After the reaction was completed, as detected by LC-MS, the reaction mixture was added to water (5 mL). The mixture was extracted with ethyl acetate (20 mL×3), and the combined organic phases were dried over anhydrous sodium sulfate, filtered and concentrated to give a crude product, which was slurried with ethyl acetate (2 mL), purified and filtered to give the target product (510 mg, 59.5%) at 25° C.

LC-MS (ESI) [M+H]⁺=390.0; ¹H NMR (400 MHz, DMSO-d₆): δ 12.24 (s, 1H), 7.92-7.90 (m, 2H), 7.69 (s, 1H), 7.60-7.59 (m, 1H), 7.41-7.40 (m, 1H), 7.30-7.28 (m, 1H), 7.20 (s, 1H), 7.17-7.16 (m, 1H), 7.10-7.09 (m, 1H), 7.04 (s, 1H), 6.85 (s, 1H), 5.52 (s, 2H), 5.14 (s, 2H).

### Step 6: Preparation of 1-(3-((1H-imidazol-1-yl)methyl)benzyl)-4-amino-1H-imidazo[4,5-c]quinolin-2(3H)-one

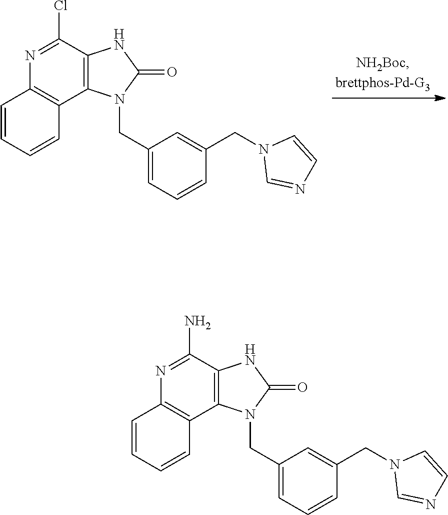 [SMILES: O=c1[nH]c2c(Cl)nc3ccccc3c2n1Cc1cccc(Cn2ccnc2)c1 ; Nc1nc2ccccc2c2c1[nH]c(=O)n2Cc1cccc(Cn2ccnc2)c1]

The starting material 1-(3-((1H-imidazol-1-yl)methyl)benzyl)-4-chloro-1H-imidazo[4,5-c]quinolin-2(3H)-one (200 mg, 513 μmol, 1.0 eq) was dissolved in tetrahydrofuran (5 mL), and tert-butyl carbamate (601 mg, 5.13 mmol, 10 eq), cesium carbonate (501 mg, 1.54 mmol, 3.0 eq), and BrettPhos-Pd-G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (46.5 mg, 51.3 μmol, 0.1 eq) were added respectively under nitrogen atmosphere. The reaction mixture was stirred at 100° C. for 4 h. After the reaction was completed, as detected by LC-MS, the reaction mixture was concentrated to remove tetrahydrofuran. Water (5 mL) was added. The mixture was extracted with ethyl acetate (10 mL×2). The combined organic phases were dried over anhydrous sodium sulfate, filtered and concentrated to give a crude product, which was separated and purified by Prep-HPLC (0.01% aqueous HCOOH solution, MeCN) to give the target compound (8.92 mg, 13.4%). LC-MS: [M+H]⁺=371.11; ¹H NMR (400 MHz, DMSO-d₆): δ 11.01 (brs, 1H), 7.68-7.65 (m, 2H), 7.53-7.51 (m, 1H), 7.31-7.29 (m, 2H), 7.27 (s, 1H), 7.22-6.98 (m, 4H), 6.86 (s, 1H), 6.34 (s, 2H), 5.43 (s, 2H), 5.15 (s, 2H).

### Example 13: Preparation of 1-(3-((1H-pyrazol-1-yl)methyl)benzyl)-4-amino-1H-imidazo[4,5-c]quinolin-2(3H)-one

### Step 1: Preparation of 3-(1H-pyrazol-1-ylmethyl)benzonitrile

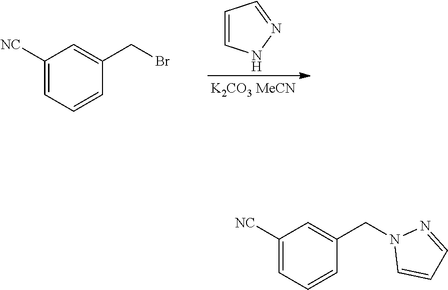 [SMILES: N#Cc1cccc(CBr)c1 ; c1cn[nH]c1 ; N#Cc1cccc(Cn2cccn2)c1]

The starting material 3-(bromomethyl)benzonitrile (14.0 g, 71.4 mmol, 1.0 eq) was dissolved in acetonitrile (150 mL), and potassium carbonate (29.6 g, 214 mmol, 3.0 eq) and pyrazole (5.35 g, 78.6 mmol, 1.1 eq) were added successively. The reaction mixture was stirred at 80° C. for 4 h. After the reaction was completed, as detected by LC-MS, the reaction mixture was filtered, and the filtrate was concentrated. Water (200 mL) was added, and the mixture was extracted with ethyl acetate (200 mL×2). The combined organic phases were dried over anhydrous sodium sulfate, filtered and concentrated to give a solid. The solid was separated and purified by flash chromatography (PE:EA=20:1 to 1:1) to give the target compound (7.10 g, 54.3%). LC-MS: [M+H]⁺=184.31; ¹H NMR (400 MHz, CDCl₃): δ 7.63-7.59 (m, 2H), 7.48-7.44 (m, 4H), 6.35-6.34 (m, 1H), 5.37 (s, 1H).

### Step 2: Preparation of (3-((1H-pyrazol-1-yl)methyl)phenyl)methylamine

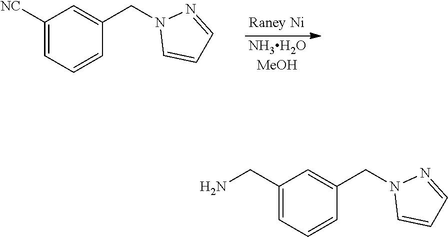 [SMILES: N#Cc1cccc(Cn2cccn2)c1 ; "NH3·H2OMeOH" ; NCc1cccc(Cn2cccn2)c1]

The starting material 3-((1H-pyrazol-1-yl)methyl)benzonitrile (500 mg, 2.73 mmol, 1.0 eq) was dissolved in methanol (3 mL) and aqueous ammonia (2 mL), and Raney nickel (234 mg) was added. The reaction mixture was purged with nitrogen 3 times, purged with hydrogen 3 times, and stirred at 25° C. for 4 h under hydrogen atmosphere (15 Psi). After the reaction was completed, as detected by TLC (DCM:MeOH=20:1), the reaction mixture was filtered, and the filtrate was concentrated to give a yellow oil. The oil was separated and purified by flash chromatography (DCM:MeOH=1:0 to 10:1) to give the target compound (320 mg, 62.6%). LC-MS: [M+H]⁺=188.01.

### Step 3: Preparation of N-(3-((1H-pyrazol-1-yl)methyl)benzyl)-2-chloro-3-nitroquinoline-4-amine

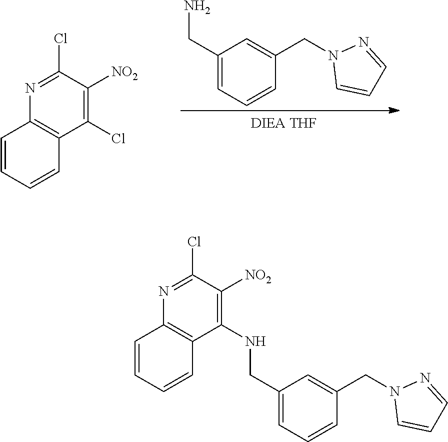 [SMILES: O=[N+]([O-])c1c(Cl)nc2ccccc2c1Cl ; NCc1cccc(Cn2cccn2)c1 ; O=[N+]([O-])c1c(Cl)nc2ccccc2c1NCc1cccc(Cn2cccn2)c1]

The starting material 2,4-dichloro-3-nitroquinoline (831 mg, 3.42 mmol, 1.0 eq) was dissolved in anhydrous tetrahydrofuran (15 mL), followed by the addition of N,N-diisopropylethylamine (1.33 g, 10.3 mmol, 3.0 eq) and (3-((1H-pyrazol-1-yl)methyl)phenyl)methylamine (640 mg, 3.42 mmol, 1.0 eq). The reaction mixture was stirred at 25° C. for 4 h. After the reaction was completed, as detected by TLC (DCM:MeOH=20:1), water (10 mL) was added. The mixture was extracted with ethyl acetate (20 mL×3). The combined organic phases were washed with saturated brine (20 mL×2), dried over anhydrous sodium sulfate, filtered and concentrated to give a crude product, which was separated and purified by flash chromatography (DCM:MeOH=1:0 to 10:1) to give the target compound (1.10 g, 81.7%). LC-MS: [M+H]⁺=394.21.

### Step 4: Preparation of N⁴-(3-((1H-pyrazol-1-yl)methyl)benzyl)-2-chloroquinoline-3,4-diamine

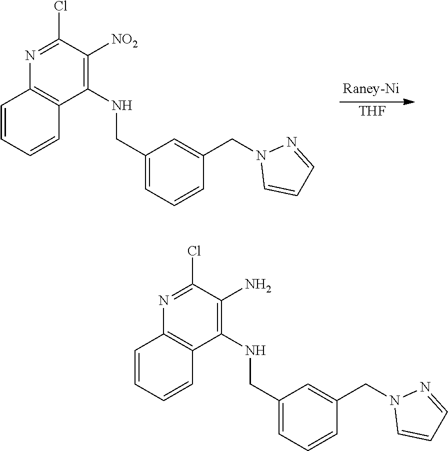 [SMILES: O=[N+]([O-])c1c(Cl)nc2ccccc2c1NCc1cccc(Cn2cccn2)c1 ; Nc1c(Cl)nc2ccccc2c1NCc1cccc(Cn2cccn2)c1 ; "THF"]

The starting material N-(3-((1H-pyrazol-1-yl)methyl)benzyl)-2-chloro-3-nitroquinolin-4-amine (1.00 g, 2.54 mmol, 1.0 eq) was dissolved in tetrahydrofuran (15 mL), and Raney nickel (218 mg) was added. The reaction mixture was purged with nitrogen 3 times, purged with hydrogen 3 times, and stirred at 25° C. for 4 h under hydrogen atmosphere (15 Psi). After the reaction was completed, as detected by TLC (DCM:MeOH=20:1), the reaction mixture was filtered, and the filtrate was concentrated to give a solid. The solid was separated and purified by flash chromatography (DCM:MeOH=1:0 to 10:1) to give the target compound (519 mg, 56.2%). LC-MS: [M+H]⁺=364.21.

### Step 5: Preparation of 1-(3-((1H-pyrazol-1-yl)methyl)benzyl)-4-chloro-1H-imidazo[4,5-c]quinolin-2(3H)-one

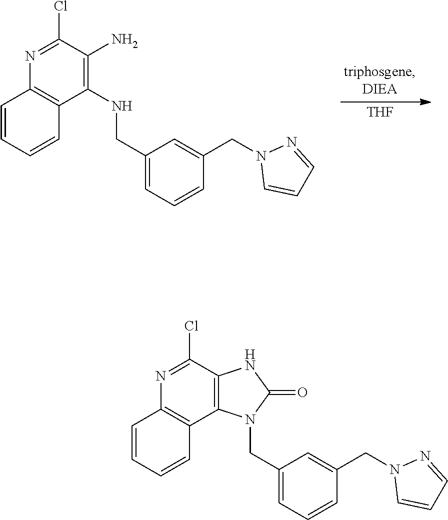 [SMILES: Nc1c(Cl)nc2ccccc2c1NCc1cccc(Cn2cccn2)c1 ; O=c1[nH]c2c(Cl)nc3ccccc3c2n1Cc1cccc(Cn2cccn2)c1 ; "THF"]

The starting material N⁴-(3-((1H-pyrazol-1-yl)methyl)benzyl)-2-chloroquinoline-3,4-diamine (519 mg, 1.43 mmol, 1.0 eq) was dissolved in anhydrous tetrahydrofuran (8 mL), followed by the addition of N,N-diisopropylethylamine (553 mg, 4.28 mmol, 3.0 eq) and triphosgene (423 mg, 1.43 mmol, 1.0 eq). The reaction mixture was stirred at 25° C. for 4 h. After the reaction was completed, as detected by LC-MS, water (5 mL) was added. The mixture was extracted with ethyl acetate (10 mL×3). The combined organic phases were washed with saturated brine (20 mL), dried over anhydrous sodium sulfate, filtered and concentrated to give a solid. The reaction mixture was poured into ethyl acetate (5 mL), slurried at 25° C. and filtered to give the target compound (315 mg, 56.7%). LC-MS: [M+H]⁺=390.10.

### Step 6: Preparation of 1-(3-((1H-pyrazol-1-yl)methyl)benzyl)-4-amino-1H-imidazo[4,5-c]quinolin-2(3H)-one

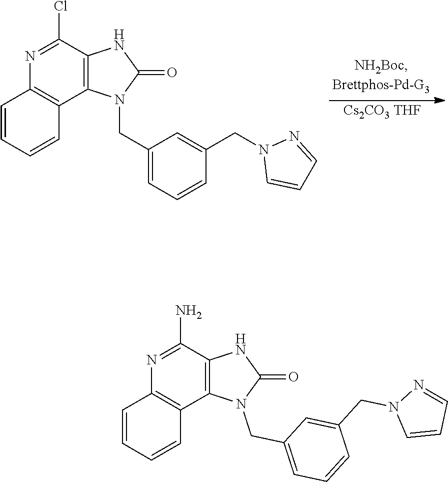 [SMILES: O=c1[nH]c2c(Cl)nc3ccccc3c2n1Cc1cccc(Cn2cccn2)c1 ; Nc1nc2ccccc2c2c1[nH]c(=O)n2Cc1cccc(Cn2cccn2)c1 ; "Cs2CO3 THF"]

The starting material 1-(3-((1H-pyrazol-1-yl)methyl)benzyl)-4-chloro-1H-imidazo[4,5-c]quinolin-2(3H)-one (200 mg, 513 μmol, 1.0 eq) was dissolved in tetrahydrofuran (8 mL), and tert-butyl carbamate (601 mg, 5.13 mmol, 10.0 eq), cesium carbonate (501 mg, 1.54 mmol, 3.0 eq), and brettphos-Pd-G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (46.5 mg, 51.3 μmol, 0.1 eq) were added successively under nitrogen atmosphere. The reaction mixture was stirred at 100° C. for 12 h. After the reaction was completed, as detected by LC-MS, the reaction mixture was filtered, and the filtrate was concentrated to give a crude product, which was separated and purified by Prep-HPLC (0.225% aqueous HCOOH solution, ACN) to give the target compound (17.5 mg, 9.21%). LC-MS: [M+H]⁺=371.32; ¹H NMR (400 MHz, DMSO-d₆): δ 11.04 (brs, 1H), 7.70-7.65 (m, 2H), 7.52 (d, J=8.0 Hz, 1H), 7.40 (s, 1H), 7.28-7.25 (m, 2H), 7.12-7.10 (m, 2H), 7.05-7.01 (m, 2H), 6.36 (brs, 2H), 6.21 (s, 1H), 5.41 (s, 2H), 5.27 (s, 2H).

### Example 14: Preparation of 4-amino-1-(3-((4-methylpiperazin-1-yl)methyl)benzyl)-1H-imidazo[4,5-c]quinolin-2(3H)-one

### Step 1: Preparation of 3-((4-methylpiperazin-1-yl)methyl)benzonitrile

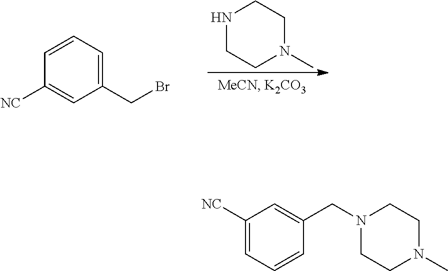

The starting material 3-(bromomethyl)benzonitrile (5.00 g, 25.5 mmol, 1.0 eq) was dissolved in acetonitrile (100 mL), and 1-methylpiperazine (3.07 g, 30.6 mmol, 3.39 mL, 1.2 eq) and potassium carbonate (10.6 g, 76.5 mmol, 3.0 eq) were added. The reaction mixture was stirred at 25° C. for 2 h. After the reaction was completed, as detected by TLC (PE:EA=10:1), the reaction mixture was concentrated to remove acetonitrile to give a crude product, and then water (30 mL) was added. The mixture was extracted with ethyl acetate (30 mL×3). The combined organic phases were washed with saturated brine, dried over anhydrous sodium sulfate, filtered and concentrated to give a crude product, which was separated and purified by flash chromatography (DCM:MeOH=30:1 to 20:1) to give the target compound (3.45 g, 62.8%). ¹H NMR (400 MHz, Methanol-d₄): δ 7.73 (s, 1H), 7.68-7.64 (m, 2H), 7.54-7.53 (m, 1H), 3.60 (s, 2H), 2.53-2.47 (m, 8H), 2.30 (s, 3H).

### Step 2: Preparation of (3-((4-methylpiperazin-1-yl)methyl)phenyl)methylamine

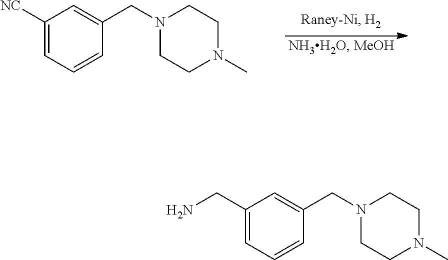

The starting material 3-((4-methylpiperazin-1-yl)methyl)benzonitrile (1.50 g, 6.97 mmol, 1.0 eq) was dissolved in methanol (25 mL), and aqueous ammonia (4.55 g, 36.4 mmol, 5 mL, 28% purity, 5.2 eq) and Raney nickel (597 mg) were added successively at 25° C. The reaction mixture was purged with nitrogen three times, purged with hydrogen three times, and then stirred at 25° C. for 1 h under hydrogen atmosphere (15 Psi). After the reaction was completed, as detected by TLC (DCM:MeOH=10:1), the reaction mixture was filtered. The filter cake was washed with methanol. The filtrate was concentrated to give the target compound (1.30 g, 85.1%), which was used directly in the next step. ¹H NMR (400 MHz, Methanol-d₄): δ 7.33-7.23 (m, 4H), 3.80 (s, 2H), 3.55 (s, 2H), 2.52-2.46 (m, 8H), 2.29 (s, 3H).

### Step 3: Preparation of 2-chloro-N-(3-((4-methylpiperazin-1-yl)methyl)benzyl)-3-nitroquinoline-4-amine

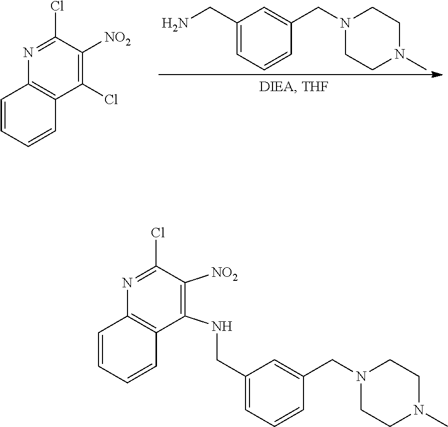 [SMILES: O=[N+]([O-])c1c(Cl)nc2ccccc2c1Cl ; CN1CCN(Cc2cccc(CN)c2)CC1 ; CN1CCN(Cc2cccc(CNc3c([N+](=O)[O-])c(Cl)nc4ccccc34)c2)CC1]

The starting material 2,4-dichloro-3-nitroquinoline (1.20 g, 4.94 mmol, 1.0 eq) was dissolved in tetrahydrofuran (20 mL), and (3-((4-methylpiperazin-1-yl)methyl)phenyl)methylamine (1.30 g, 5.92 mmol, 1.2 eq) and N,N-diisopropylethylamine (1.91 g, 14.8 mmol, 3.0 eq) were added. The reaction mixture was stirred at 25° C. for 13 h. After the reaction was completed, as detected by LC-MS, the reaction mixture was concentrated to remove tetrahydrofuran to give a crude product. Water (20 mL) was added to the crude product. The mixture was extracted with dichloromethane (20 mL×3). The combined organic phases were dried over anhydrous sodium sulfate, filtered and concentrated to give a crude product, which was separated and purified by flash chromatography (DCM:MeOH=96:4 to 95:5) to give the target compound (1.20 g, 57.1%). LC-MS: [M+H]⁺=426.12.

### Step 4: Preparation of 2-chloro-N⁴-(3-((4-methylpiperazin-1-yl)methyl)benzyl)quinoline-3,4-diamine

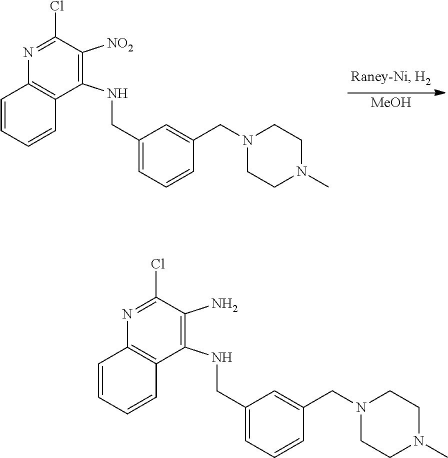 [SMILES: "MeOH" ; CN1CCN(Cc2cccc(CNc3c([N+](=O)[O-])c(Cl)nc4ccccc34)c2)CC1 ; CN1CCN(Cc2cccc(CNc3c(N)c(Cl)nc4ccccc34)c2)CC1]

The starting material 2-chloro-N-(3-((4-methylpiperazin-1-yl)methyl)benzyl)-3-nitroquinolin-4-amine (1.20 g, 2.82 mmol, 1 eq) was dissolved in methanol (15 mL), and Raney nickel (241 mg) was added at 25° C. The reaction mixture was purged with nitrogen three times, purged with hydrogen three times, and then stirred at 25° C. for 1.5 h under hydrogen atmosphere (15 Psi). After the reaction was completed, as detected by LC-MS, the reaction mixture was filtered. The filter cake was washed with methanol (15 mL). The filtrate was concentrated to give a light yellow crude product. The crude product was separated and purified by flash chromatography (DCM:MeOH=30:1 to 15:1) to give the target compound (550 mg, 49.3%). LC-MS: [M+H]⁺=396.32.

### Step 5: Preparation of 4-chloro-1-(3-((4-methylpiperazin-1-yl)methyl)benzyl)-1H-imidazo[4,5-c]quinolin-2(3H)-one

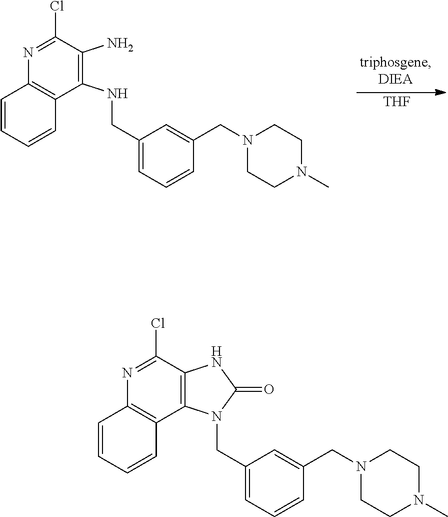 [SMILES: CN1CCN(Cc2cccc(CNc3c(N)c(Cl)nc4ccccc34)c2)CC1 ; CN1CCN(Cc2cccc(Cn3c(=O)[nH]c4c(Cl)nc5ccccc5c43)c2)CC1 ; "THF"]

The starting material 2-chloro-N⁴-(3-((4-methylpiperazin-1-yl)methyl)benzyl)quinoline-3,4-diamine (550 mg, 1.39 mmol, 1.0 eq) was dissolved in tetrahydrofuran (10 mL), and N,N-diisopropylethylamine (539 mg, 4.17 mmol, 3.0 eq) and triphosgene (450 mg, 1.52 mmol, 1.1 eq) were added successively. The reaction mixture was stirred at 25° C. for 2 h. After the reaction was completed, as detected by LC-MS, the reaction mixture was concentrated to give a crude product. Water (10 mL) was added to the crude product. The mixture was extracted with ethyl acetate (10 mL×3), and the organic phase was discarded. The aqueous phase was adjusted to pH 9 with saturated aqueous sodium bicarbonate solution and extracted with dichloromethane (10 mL×3). The combined organic phases were dried over anhydrous sodium sulfate, filtered and concentrated to give the target compound (320 mg, 54.6%), which was used directly in the next step. LC-MS: [M+H]⁺=422.21.

### Step 6: Preparation of 4-amino-1-(3-((4-methylpiperazin-1-yl)methyl)benzyl)-1H-imidazo[4,5-c]quinolin-2(3H)-one

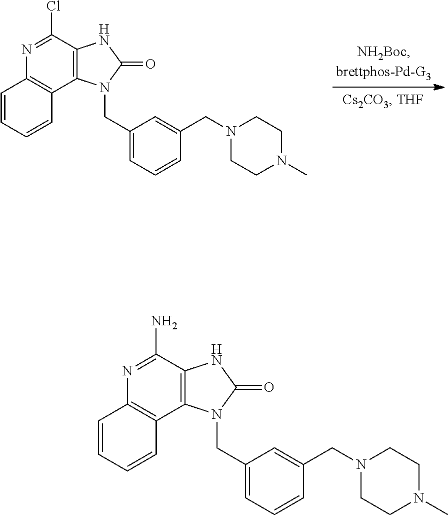 [SMILES: CN1CCN(Cc2cccc(Cn3c(=O)[nH]c4c(Cl)nc5ccccc5c43)c2)CC1 ; CN1CCN(Cc2cccc(Cn3c(=O)[nH]c4c(N)nc5ccccc5c43)c2)CC1]

The starting material 4-chloro-1-(3-((4-methylpiperazin-1-yl)methyl)benzyl)-1H-imidazo[4,5-c]quinolin-2(3H)-one (100 mg, 237 μmol, 1.0 eq) was dissolved in tetrahydrofuran (2 mL), and tert-butyl carbamate (278 mg, 2.37 mmol, 10 eq), cesium carbonate (232 mg, 711 μmol, 3.0 eq), and brettphos-Pd-G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (21.5 mg, 23.7 μmol, 0.1 eq) were added respectively under nitrogen atmosphere. The reaction mixture was stirred at 100° C. for 17 h. After the reaction was completed, as detected by LC-MS, the reaction mixture was filtered to remove cesium carbonate. Water (5 mL) was added to the filtrate for dilution, then the pH was adjusted to 5 with 1 N diluted hydrochloric acid. The mixture was extracted with ethyl acetate (10 mL×2). The organic phase was discarded. The aqueous phase was adjusted to pH 9 with saturated aqueous sodium bicarbonate solution and extracted with dichloromethane (10 mL×3). The combined organic phases were dried over anhydrous sodium sulfate, filtered and concentrated to give a crude product, which was separated and purified by Prep-HPLC (0.05% aqueous HCOOH solution, MeCN) to give the target compound (33.5 mg, 35.1%). LC-MS: [M+H]⁺=403.01; ¹H NMR (400 MHz, Methanol-d₄): δ 7.95 (d, J=8.4 Hz, 1H), 7.75-7.73 (m, 1H), 7.70-7.68 (m, 1H), 7.54 (s, 1H), 7.47-7.43 (m, 3H), 7.42-7.41 (m, 1H), 5.69 (s, 2H), 4.19 (s, 2H), 3.65-3.31 (m, 6H), 2.94 (s, 3H), 2.76-2.73 (m, 2H).

### Example 15: Preparation of 4-amino-1-(3-(morpholinomethyl)benzyl)-1H-imidazo[4,5-c]quinolin-2(3H)-one

### Step 1: Preparation of 3-(morpholinomethyl)benzonitrile

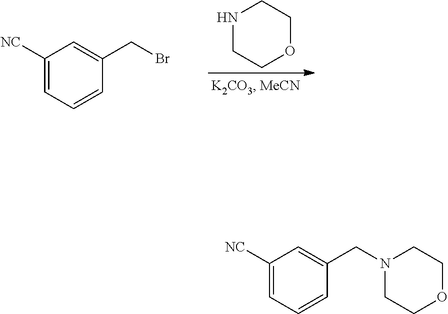 [SMILES: N#Cc1cccc(CBr)c1 ; C1COCCN1 ; N#Cc1cccc(CN2CCOCC2)c1]

The starting material 3-(bromomethyl)benzonitrile (5.00 g, 25.5 mmol, 1.0 eq) was dissolved in acetonitrile (50 mL), and morpholine (2.44 g, 28.1 mmol, 2.47 mL, 1.1 eq) and potassium carbonate (10.6 g, 76.5 mmol, 3.0 eq) were added. The reaction mixture was stirred at 25° C. for 1 h. After the reaction was completed, as detected by LC-MS, water (100 mL) was added to the reaction mixture. The mixture was extracted with ethyl acetate (100 mL×2). The combined organic phases were washed with saturated brine (150 mL), dried over anhydrous sodium sulfate, filtered and concentrated to give a crude product, which was separated and purified by flash chromatography (DCM:MeOH=1:0 to 100:1) to give the target compound (2.00 g, 38.8%). LC-MS: [M+H]⁺=203.31.

### Step 2: Preparation of (3-(morpholinomethyl)phenyl)methylamine

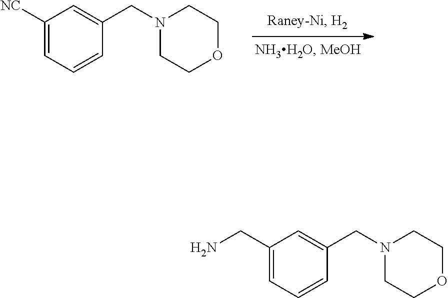 [SMILES: N#Cc1cccc(CN2CCOCC2)c1 ; NCc1cccc(CN2CCOCC2)c1]

The starting material 3-(morpholinomethyl)benzonitrile (1.50 g, 6.97 mmol, 1.0 eq) was dissolved in methanol (10 mL), and aqueous ammonia (2 mL) and Raney nickel (424 mg) were added successively at 25° C. The reaction mixture was purged with nitrogen three times, purged with hydrogen three times, and stirred at 25° C. for 1 h under hydrogen atmosphere (15 Psi). After the reaction was completed, as detected by TLC (DCM:MeOH=10:1), the reaction mixture was filtered, the filter cake was washed with methanol, and the filtrate was concentrated to give the target compound (900 mg, 85.2%), which was used directly in the next step.

### Step 3: Preparation of 2-chloro-N-(3-(morpholinomethyl)benzyl)-3-nitroquinoline-4-amine

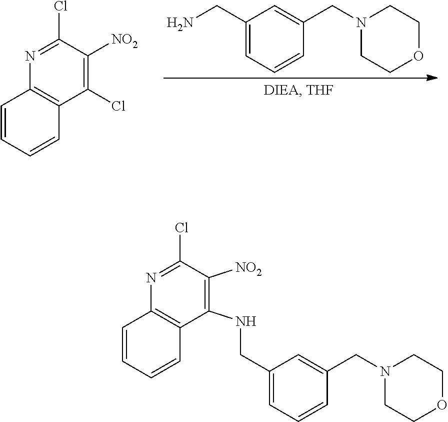 [SMILES: O=[N+]([O-])c1c(Cl)nc2ccccc2c1Cl ; NCc1cccc(CN2CCOCC2)c1 ; O=[N+]([O-])c1c(Cl)nc2ccccc2c1NCc1cccc(CN2CCOCC2)c1]

The starting material 2,4-dichloro-3-nitroquinoline (880 mg, 3.29 mmol, 1.0 eq) was dissolved in tetrahydrofuran (2 mL), and (3-(morpholinomethyl)phenyl)methylamine (747 mg, 3.62 mmol, 1.1 eq) and N,N-diisopropylethylamine (1.28 g, 9.87 mmol, 3.0 eq) were added. The reaction mixture was stirred at 25° C. for 3 h. After the reaction was completed, as detected by LC-MS, the reaction mixture was concentrated to remove tetrahydrofuran to give a crude product. Then water (20 mL) was added, and the mixture was extracted with ethyl acetate (20 mL×2). The combined organic phases were dried over anhydrous sodium sulfate, filtered and concentrated to give a crude product (1.20 g, 57.1%), which was used directly in the next step. LC-MS: [M+H]⁺=413.31.

### Step 4: Preparation of 2-chloro-N⁴-(3-(morpholinomethyl)benzyl)quinoline-3,4-diamine

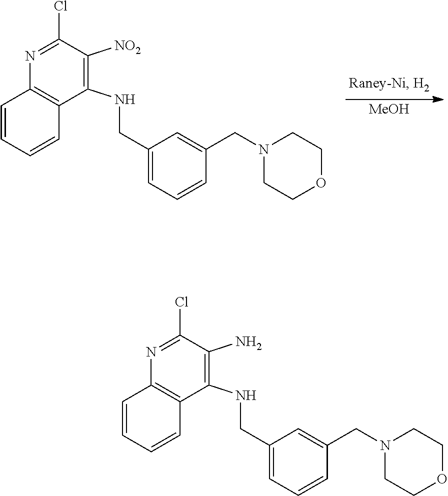 [SMILES: "MeOH" ; O=[N+]([O-])c1c(Cl)nc2ccccc2c1NCc1cccc(CN2CCOCC2)c1 ; Nc1c(Cl)nc2ccccc2c1NCc1cccc(CN2CCOCC2)c1]

The starting material 2-chloro-N-(3-(morpholinomethyl)benzyl)-3-nitroquinolin-4-amine (1.40 g, 3.39 mmol, 1.0 eq) was dissolved in methanol (15 mL), and Raney nickel (291 mg, 2.39 mmol, 1.0 eq) were added at 25° C. The reaction mixture was purged with nitrogen three times, purged with hydrogen three times, and stirred at 25° C. for 1 h under hydrogen atmosphere (15 Psi). After the reaction was completed, as detected by LC-MS, the reaction mixture was filtered. The filter cake was washed with methanol (15 mL). The filtrate was concentrated to give a yellow crude product. The crude product was separated and purified by flash chromatography (DCM:MeOH=100:1 to 30:1) to give the target compound (800 mg, 61.6%). LC-MS: [M+H]⁺=383.32.

### Step 5: Preparation of 4-chloro-1-(3-(morpholinomethyl)benzyl)-1H-imidazo[4,5-c]quinolin-2(3H)-one

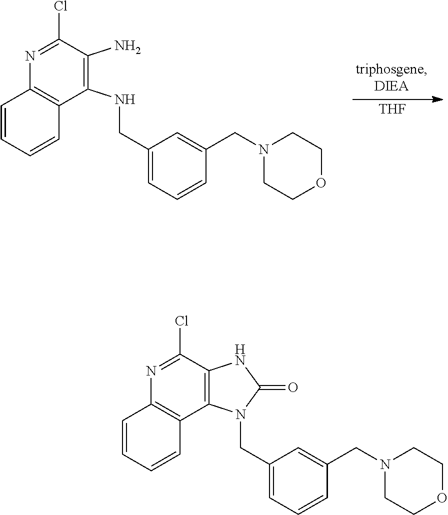 [SMILES: Nc1c(Cl)nc2ccccc2c1NCc1cccc(CN2CCOCC2)c1 ; O=c1[nH]c2c(Cl)nc3ccccc3c2n1Cc1cccc(CN2CCOCC2)c1 ; "THF"]

The starting material 2-chloro-N⁴-(3-(morpholinomethyl)benzyl)quinoline-3,4-diamine (750 mg, 1.96 mmol, 1.0 eq) was dissolved in tetrahydrofuran (10 mL), and N,N-diisopropylethylamine (759 mg, 5.88 mmol, 3.0 eq) and triphosgene (581 mg, 1.96 mmol, 1.0 eq) were added successively. The reaction mixture was stirred at 25° C. for 3 h. After the reaction was completed, as detected by LC-MS, the reaction mixture was concentrated to give a crude product. Water (10 mL) was added to the crude product, and the mixture was adjusted to pH 9 with saturated aqueous sodium bicarbonate solution and extracted with dichloromethane (10 mL×3). The combined organic phases were dried over anhydrous sodium sulfate, filtered and concentrated to give a crude product (320 mg, 54.6%), which was used directly in the next step without purification. LC-MS: [M+H]⁺=409.30.

### Step 6: Preparation of 4-amino-1-(3-(morpholinomethyl)benzyl)-1H-imidazo[4,5-c]quinolin-2(3H)-one

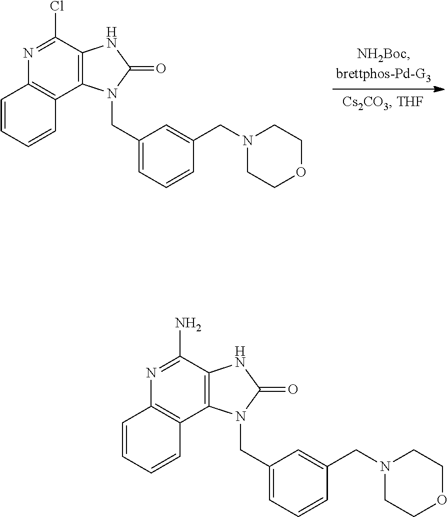 [SMILES: O=c1[nH]c2c(Cl)nc3ccccc3c2n1Cc1cccc(CN2CCOCC2)c1 ; Nc1nc2ccccc2c2c1[nH]c(=O)n2Cc1cccc(CN2CCOCC2)c1]

The starting material 4-chloro-1-(3-(morpholinomethyl)benzyl)-1H-imidazo[4,5-c]quinolin-2(3H)-one (200 mg, 489.14 μmol, 1.0 eq) was dissolved in tetrahydrofuran (4 mL), and tert-butyl carbamate (573 mg, 4.89 mmol, 10 eq), cesium carbonate (478 mg, 1.47 mmol, 3.0 eq), and brettphos-Pd-G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (44.3 mg, 48.9 μmol, 0.1 eq) were added respectively under nitrogen atmosphere. The reaction mixture was stirred at 100° C. for 17 h. After the reaction was completed, as detected by LC-MS, the reaction mixture was filtered to remove cesium carbonate. Water (10 mL) was added for dilution, the pH was adjusted to 5 with 1 M (molar concentration) diluted hydrochloric acid. The mixture was extracted with ethyl acetate (10 mL×3). The organic phase was discarded. The aqueous phase was adjusted to pH 9 with saturated aqueous sodium bicarbonate solution and extracted with dichloromethane (10 mL×3). The organic phases were dried over anhydrous sodium sulfate, filtered and concentrated to give a crude product, which was separated and purified by Prep-HPLC (0.01% aqueous HCl solution, MeCN) to give the target compound (60.0 mg, 31.5%). LC-MS: [M+H]⁺=390.01; ¹H NMR (400 MHz, DMSO-d₆): δ 13.90 (brs, 1H), 12.22 (brs, 1H), 10.81 (brs, 1H), 8.52 (s, 1H), 7.80-7.75 (m, 2H), 7.63-7.61 (m, 1H), 7.46-7.42 (m, 4H), 7.32-7.30 (m, 1H), 5.58 (s, 2H), 4.23 (brs, 2H), 3.82-3.79 (m, 2H), 3.61-3.58 (m, 2H), 2.99-2.96 (m, 2H), 2.91-2.87 (m, 2H).

### Example 16: Preparation of 4-amino-1-(2-(pyrrolidin-1-ylmethyl)benzyl)-1H-imidazo[4,5-c]quinolin-2(3H)-one

### Step 1: Preparation of 2-chloro-3-nitro-N-(2-(pyrrolidin-1-ylmethyl)benzyl)quinoline-4-amine

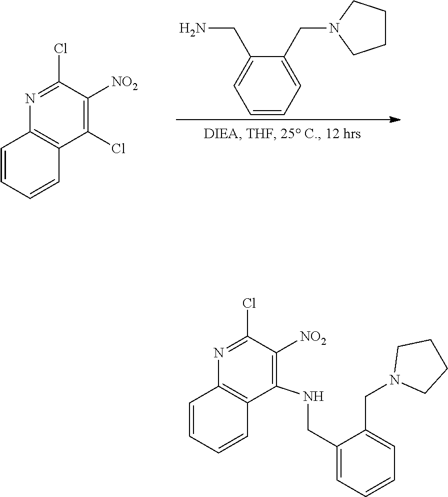 [SMILES: O=[N+]([O-])c1c(Cl)nc2ccccc2c1Cl ; NCc1ccccc1CN1CCCC1 ; O=[N+]([O-])c1c(Cl)nc2ccccc2c1NCc1ccccc1CN1CCCC1]

The starting material 2,4-dichloro-3-nitroquinoline (1.00 g, 4.11 mmol, 1.0 eq) was dissolved in anhydrous tetrahydrofuran (10 mL), and then NA-diisopropylethylamine (1.60 g, 12.3 mmol, 3.0 eq) and (2-(pyrrolidin-1-ylmethyl)phenyl)methylamine (783 mg, 4.11 mmol, 1.0 eq) were added. The reaction mixture was stirred at 25° C. for 4 h. After the reaction was completed, as detected by LC-MS, water (10 mL) was added, and then the mixture was extracted with ethyl acetate (20 mL×3). The combined organic phases were washed with saturated brine (30 mL), dried over anhydrous sodium sulfate, filtered and concentrated to give a solid. The solid was separated and purified by flash chromatography (DCM:MeOH=1:0 to 10:1) to give the target compound (760 mg, 46.5%). LC-MS: [M+H]⁺=397.01.

### Step 2: Preparation of 2-chloro-N⁴-(2-(pyrrolidin-1-ylmethyl)benzyl)quinolin-3,4-diamine

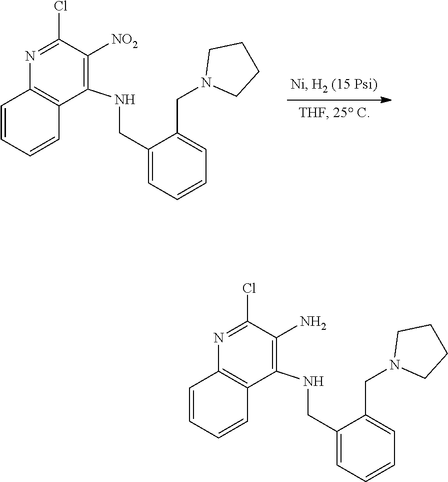 [SMILES: O=[N+]([O-])c1c(Cl)nc2ccccc2c1NCc1ccccc1CN1CCCC1 ; Nc1c(Cl)nc2ccccc2c1NCc1ccccc1CN1CCCC1]

The starting material 2-chloro-3-nitro-N-(2-(pyrrolidin-1-ylmethyl)benzyl)quinolin-4-amine (760 mg, 1.91 mmol, 1.0 eq) was dissolved in tetrahydrofuran (10 mL). Raney nickel (164 mg) was added. The reaction mixture was purged with nitrogen 3 times, purged with hydrogen 3 times and stirred at 25° C. for 4 h under hydrogen atmosphere (15 Psi). After the reaction was completed, as detected, the reaction mixture was filtered and concentrated to give a solid. The solid was separated and purified by flash chromatography (DCM:MeOH=1:0 to 10:1) to give the target compound (510 mg, 72.6%). LC-MS: [M+H]⁺=367.01.

### Step 3: Preparation of 4-chloro-1-(2-(pyrrolidin-1-ylmethyl)benzyl)-1H-imidazo[4,5-c]quinolin-2(3H)-one

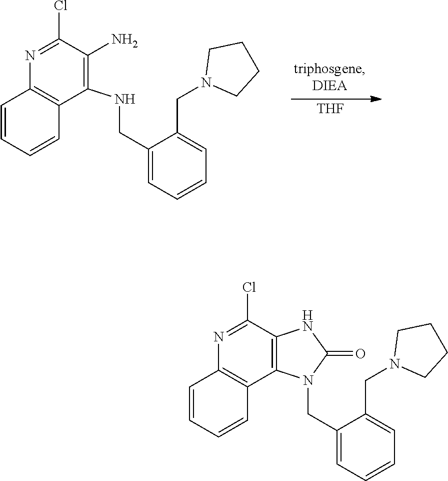 [SMILES: Nc1c(Cl)nc2ccccc2c1NCc1ccccc1CN1CCCC1 ; O=c1[nH]c2c(Cl)nc3ccccc3c2n1Cc1ccccc1CN1CCCC1 ; "THF"]

The starting material 2-chloro-N⁴-(2-(pyrrolidin-1-ylmethyl)benzyl)quinolin-3,4-diamine (510 mg, 1.39 mmol, 1.0 eq) was dissolved in anhydrous tetrahydrofuran (10 mL), and then N,N-diisopropylethylamine (539 mg, 4.17 mmol, 3.0 eq) and triphosgene (413 mg, 1.39 mmol, 1.0 eq) were added. The reaction mixture was stirred at 25° C. for 4 h. After the reaction was completed, as detected by LC-MS, water (10 mL) was added. The mixture was extracted with ethyl acetate (15 mL×3). The combined organic phases were washed with saturated brine (20 mL), dried over anhydrous sodium sulfate, filtered and concentrated to give a solid. The solid was added to ethyl acetate (5 mL), slurried at 25° C. and filtered to give the target compound (210 mg, 38.5%). LC-MS: [M+H]⁺=393.21.

### Step 4: Preparation of 4-amino-1-(2-(pyrrolidin-1-ylmethyl)benzyl)-1H-imidazo[4,5-c]quinolin-2(3H)-one

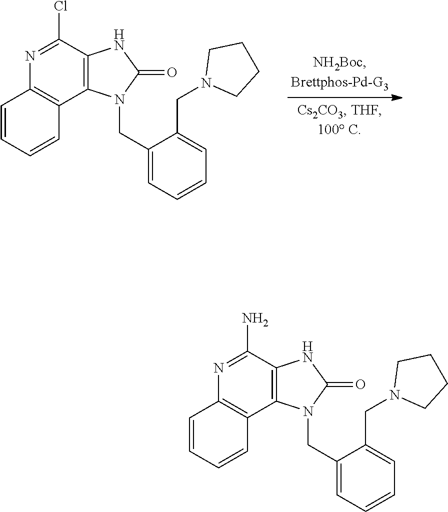 [SMILES: O=c1[nH]c2c(Cl)nc3ccccc3c2n1Cc1ccccc1CN1CCCC1 ; Nc1nc2ccccc2c2c1[nH]c(=O)n2Cc1ccccc1CN1CCCC1]

The starting material 4-chloro-1-(2-(pyrrolidin-1-ylmethyl)benzyl)-1H-imidazo[4,5-c]quinolin-2(3H)-one (105 mg, 267 μcool, 1.0 eq) was dissolved in tetrahydrofuran (2 mL). Tert-butyl carbamate (313, 2.67 mmol, 10.0 eq), cesium carbonate (261 mg, 802 mmol, 3.0 eq), and brettphos-Pd-G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (24.2 mg, 26.7 μcool, 0.1 eq) were added sequentially under nitrogen atmosphere. The reaction mixture was stirred at 100° C. for 17 h. After the reaction was completed, as detected by LC-MS, the reaction mixture was filtered, and the filtrate was concentrated to give a crude product, which was separated and purified by Prep-HPLC (0.05% aqueous HCl solution, ACN) to give the target compound (12.1 mg, 12.0%). LC-MS: [M+H]⁺=374.01; ¹H NMR (400 MHz, DMSO-d₆): δ 12.27 (s, 1H), 10.54 (s, 1H), 8.52 (s, 1H), 7.80-7.75 (m, 3H), 7.66-7.64 (m, 1H), 7.39 (t, J=6.0 Hz, 1H), 7.32 (t, J=8.0 Hz, 1H), 7.26 (t, J=8.0 Hz, 1H), 6.83 (d, J=7.6 Hz, 1H), 5.80 (s, 2H), 4.70 (s, 2H), 3.60-3.51 (m, 2H), 3.38-3.30 (m, 2H), 2.12 (brs, 2H), 2.02-1.97 (brs, 2H).

### Example 17: Preparation of 4-amino-1-(3-((diethylamino)methyl)benzyl)-1H-imidazo[4,5-c]quinolin-2(3H)-one

### Step 1: Preparation of 3-((diethylamino)methyl)benzonitrile

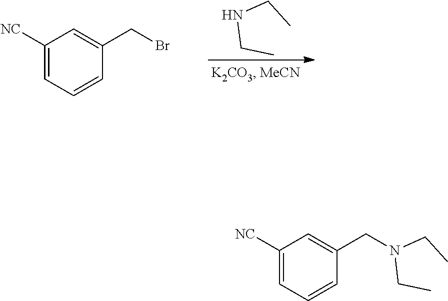

The starting material 3-(bromomethyl)benzonitrile (5.00 g, 25.5 mmol, 1.0 eq) was dissolved in acetonitrile (50 mL). Potassium carbonate (10.6 g, 76.5 mmol, 3.0 eq) and diethylamine (1.87 g, 25.5 mmol, 2.63 mL, 1.0 eq) were added sequentially. The reaction mixture was stirred at 25° C. for 1 h. After the reaction was completed, as detected by LC-MS, water (100 mL) was added to the reaction mixture. The mixture was extracted with ethyl acetate (100 mL×2). The combined organic phases were dried over anhydrous sodium sulfate, filtered and concentrated to give a crude product, which was separated and purified by flash chromatography (DCM:MeOH=1:0 to 100:1) to give the target compound (7.10 g, 54.3%). LC-MS: [M+H]⁺=189.31; ¹H NMR (400 MHz, CDCl₃): δ 7.59 (s, 1H), 7.50 (d, J=7.6 Hz, 1H), 7.44 (d, J=7.6 Hz, 1H), 7.32 (t, J=7.6 Hz, 1H), 3.49 (s, 2H), 2.46-2.41 (t, J=7.0 Hz, 4H), 0.96 (t, J=7.0 Hz, 6H).

### Step 2: Preparation of N-(3-(aminomethyl)benzyl)-N-ethylethylamine

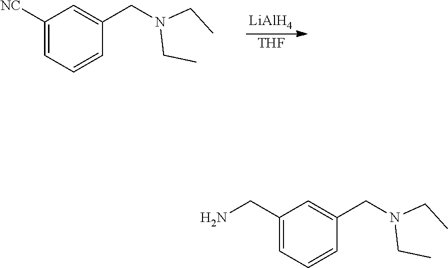

3-((diethylamino)methyl)benzonitrile (2.00 g, 10.6 mmol, 1.0 eq) was dissolved in tetrahydrofuran (20 mL). Lithium aluminium hydride (806 mg, 21.2 mmol, 2.0 eq) was added slowly at 0° C. The reaction mixture was stirred at 25° C. for 1 h. After the reaction was completed, as detected by TLC (DCM:MeOH=1:1), water (0.80 mL), 15% aqueous NaOH solution (0.80 mL), and water (2.40 mL) were added sequentially to the reaction mixture. The mixture was dried over anhydrous sodium sulfate, filtered and concentrated to give the target compound (1.80 g, 88.1%). The crude product was used directly in the next step.

### Step 3: Preparation of 2-chloro-N-(3-((di ethyl amino)methyl)benzyl)-3-nitroquinoline-4-amine

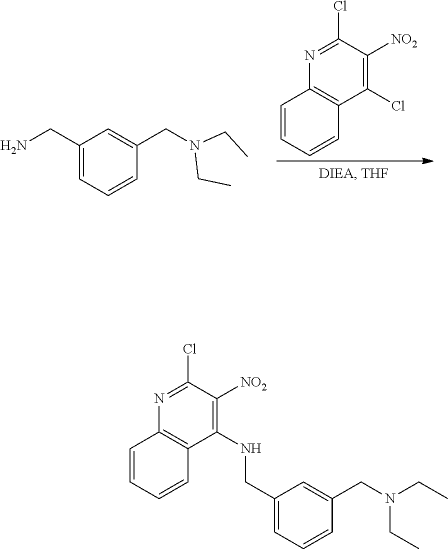

The starting material 2,4-dichloro-3-nitroquinoline (1.00 g, 4.11 mmol, 1.0 eq) was dissolved in anhydrous tetrahydrofuran (10 mL), and then NA-diisopropylethylamine (1.60 g, 12.3 mmol, 3.0 eq) and N-(3-(aminomethyl)benzyl)-N-ethylethylamine (870 mg, 4.53 mmol, 1.1 eq) were added. The reaction mixture was stirred at 25° C. for 3 h. After the reaction was completed, as detected by LC-MS, water (20 mL) was added. The mixture was extracted with ethyl acetate (10 mL×3). The combined organic phases were washed with saturated brine (10 mL), dried over anhydrous sodium sulfate, filtered and concentrated to give the target compound (1.40 g, 85.3%). LC-MS: [M+H]⁺=399.11.

### Step 4: Preparation of 2-chloro-N⁴-(3-((di ethyl amino)methyl)benzyl)quinolin-3,4-diamine

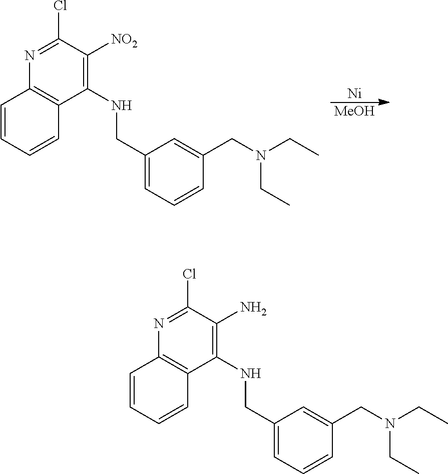

The starting material 2-chloro-N-(3-((di ethyl amino)methyl)benzyl)-3-nitroquinolin-4-amine (1.40 g, 351 mmol, 1.0 eq) was dissolved in methanol (25 mL). Raney nickel (301 mg) was added. The reaction mixture was purged with nitrogen 3 times, purged with hydrogen 3 times and stirred at 25° C. for 16 h under hydrogen atmosphere (15 Psi). After the reaction was completed, as detected by LC-MS, the reaction mixture was filtered, and the filtrate was concentrated to give a crude product, which was separated and purified by flash chromatography (DCM:MeOH=100:0 to 95:5) to give the target compound (400 mg, 31.0%). LC-MS: [M+H]⁺=369.11.

### Step 5: Preparation of 4-chloro-1-(3-((diethylamino)methyl)benzyl)-1H-imidazo[4,5-c]quinolin-2(3H)-one

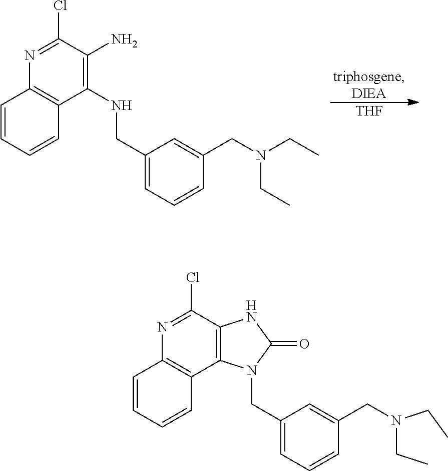 [SMILES: CCN(CC)Cc1cccc(CNc2c(N)c(Cl)nc3ccccc23)c1 ; "triphosgene,DIEA" ; CCN(CC)Cc1cccc(Cn2c(=O)[nH]c3c(Cl)nc4ccccc4c32)c1 ; "THF"]

The starting material 2-chloro-N⁴-(3-((diethylamino)methyl)benzyl)quinolin-3,4-diamine (400 mg, 1.08 mmol, 1.0 eq) was dissolved in anhydrous tetrahydrofuran (5.0 mL), and then N,N-diisopropylethylamine (420 mg, 3.25 mmol, 3.0 eq) and triphosgene (322 mg, 1.08 mmol, 1.0 eq) were added. The reaction mixture was stirred at 25° C. for 1 h. After the reaction was completed, as detected by LC-MS, the reaction mixture was concentrated, added with water (10 mL) for dilution, and extracted with dichloromethane (10 mL×2). The aqueous phase was made basic with sodium bicarbonate and extracted with dichloromethane (10 mL×2) to give the organic phase. The organic phase was concentrated to give a solid, which is the target compound (500 mg, 93.4%). LC-MS: [M+H]⁺=395.11.

### Step 6: Preparation of 4-amino-1-(3-((diethylamino)methyl)benzyl)-1H-imidazo[4,5-c]quinolin-2(3H)-one

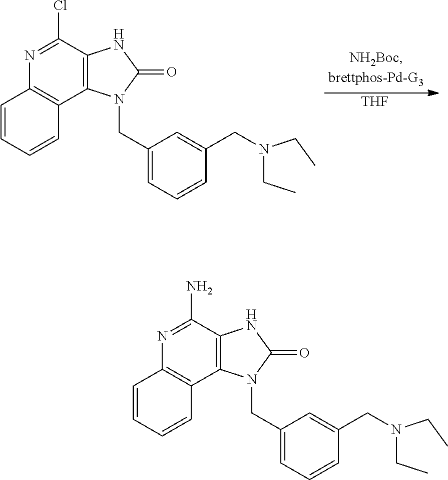 [SMILES: CCN(CC)Cc1cccc(Cn2c(=O)[nH]c3c(Cl)nc4ccccc4c32)c1 ; CCN(CC)Cc1cccc(Cn2c(=O)[nH]c3c(N)nc4ccccc4c32)c1 ; "THF"]

The starting material 4-chloro-1-(3-((diethylamino)methyl)benzyl)-1H-imidazo[4,5-c]quinolin-2(3H)-one (100 mg, 253 μmol, 1.0 eq) was dissolved in tetrahydrofuran (2.0 mL). Tert-butyl carbamate (297 mg, 2.53 mmol, 10.0 eq), cesium carbonate (248 mg, 0.76 mmol, 3.0 eq), and brettphos-Pd-G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (22.9 mg, 25.3 μmol, 0.1 eq) were added sequentially under nitrogen atmosphere. The reaction mixture was stirred at 100° C. for 16 h. After the reaction was completed, as detected by LC-MS, the mixture was filtered and concentrated to give a crude product, which was separated and purified by Prep-HPLC (0.01% aqueous HCOOH, MeCN) to give the target compound (23 mg, 23.7%). LC-MS: [M+H]⁺=376.41; ¹H NMR (400 MHz, DMSO-d₆): δ 7.69 (d, J=8.0 Hz, 1H), 7.50 (d, J=8.4 Hz, 1H), 7.29-7.25 (m, 1H), 7.24-7.21 (m, 2H), 7.15-7.14 (m, 1H), 7.05 (d, J=8.0 Hz, 1H), 7.00-6.98 (m, 1H), 6.34 (s, 2H), 5.44 (s, 2H), 3.45 (s, 2H), 2.33 (q, J=6.8 Hz, 4H), 0.84 (t, J=6.8 Hz, 6H).

### Example 18: Preparation of 4-amino-1-(3-((tert-butylamino)methyl)benzyl)-1H-imidazo[4,5-c]quinolin-2(3H)-one

### Step 1: Preparation of 3-((tert-butylamino)methyl)benzonitrile

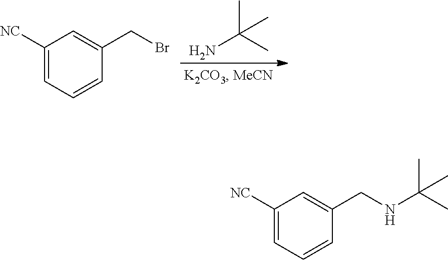

The starting material 3-(bromomethyl)benzonitrile (5.00 g, 25.5 mmol, 1.0 eq) was dissolved in acetonitrile (50 mL). Potassium carbonate (10.6 g, 76.5 mmol, 3.0 eq) and tert-butylamine (2.05 g, 28.1 mmol, 1.1 eq) were added sequentially. The reaction mixture was stirred at 25° C. for 1 h. After the reaction was completed, as detected by LC-MS, the reaction mixture was added to water (100 mL). The mixture was extracted with ethyl acetate (100 mL×2). The combined organic phases were washed with saturated brine, dried over anhydrous sodium sulfate, filtered and concentrated to give a solid. The solid was separated and purified by flash chromatography (DCM:MeOH=1:0 to 100:1) to give the target compound (3.50 g, 72.9%). LC-MS: [M+H]⁺=189.31.

### Step 2: Preparation of 3-((tert-butylamino)methyl)benzylamine

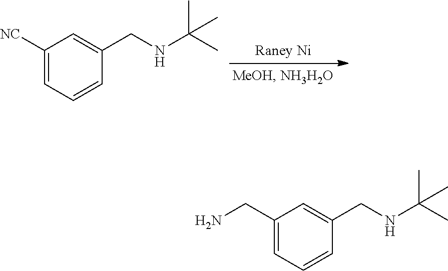

The starting material 3-((tert-butylamino)methyl)benzonitrile (200 mg, 1.06 mmol, 1.0 eq) was dissolved in methanol (2 mL). Raney nickel (91.0 mg) was added. The reaction mixture was purged with nitrogen 3 times, purged with hydrogen 3 times and stirred at 25° C. for 4 h under hydrogen atmosphere (40 psi). After the reaction was completed, as detected by LC-MS, the reaction mixture was filtered and concentrated to give the target compound (150 mg, 73.4%). The crude product was used directly in the next step. LC-MS: [M+H]⁺=189.01.

### Step 3: Preparation of N-(3-((tert-butylamino)methyl)benzyl)-2-chloro-3-nitroquinoline-4-amine

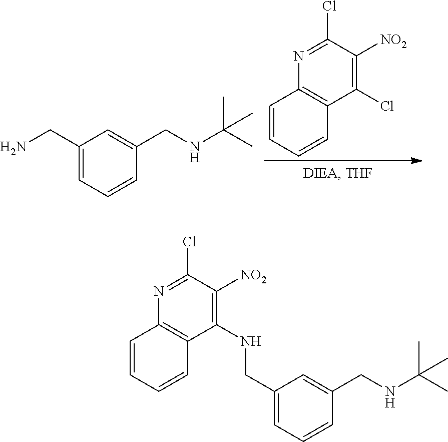

The starting material 2,4-dichloro-3-nitroquinoline (1.42 g, 5.82 mmol, 1.0 eq) was dissolved in anhydrous tetrahydrofuran (25 mL), and then N,N-diisopropylethylamine (2.26 g, 17.5 mmol, 3.0 eq) and 3-((tert-butylamino)methyl)benzylamine (1.40 g, 5.82 mmol, 1.0 eq) were added. The reaction mixture was stirred at 25° C. for 8 h. After the reaction was completed, as detected by LC-MS, the reaction mixture was concentrated. Water (15 mL) was added to the reaction mixture. The mixture was extracted with dichloromethane (10 mL×3). The combined organic phases were washed with saturated brine (15 mL), dried over anhydrous sodium sulfate, filtered and concentrated to give the target compound (2.00 g, 86.1%). LC-MS: [M+H]⁺=399.01.

### Step 4: Preparation of N⁴-(3-((tert-butylamino)methyl)benzyl)-2-chloroquinoline-3,4-diamine

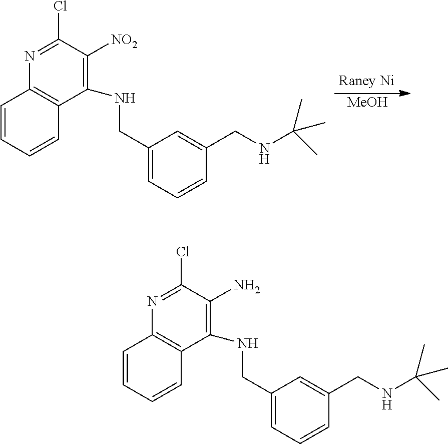 [SMILES: "MeOH" ; CC(C)(C)NCc1cccc(CNc2c([N+](=O)[O-])c(Cl)nc3ccccc23)c1 ; CC(C)(C)NCc1cccc(CNc2c(N)c(Cl)nc3ccccc23)c1]

The starting material N-(3-((tert-butylamino)methyl)benzyl)-2-chloro-3-nitroquinolin-4-amine (1.70 g, 4.26 mmol, 1.0 eq) was dissolved in tetrahydrofuran (10 mL). Raney nickel (365 mg) was added. The reaction mixture was purged with nitrogen 3 times, purged with hydrogen 3 times and stirred at 25° C. for 3 h under hydrogen atmosphere (15 Psi). After the reaction was completed, as detected by LC-MS, the reaction mixture was filtered, and the filtrate was concentrated to give a solid. The solid was separated and purified by flash chromatography (DCM:MeOH=100:0 to 20:1) to give the target compound (900 mg, 40.1%). LC-MS: [M+H]⁺=369.01.

### Step 5: Preparation of 1-(3-((tert-butylamino)methyl)benzyl)-4-chloro-1H-imidazo[4,5-c]quinolin-2(3H)-one

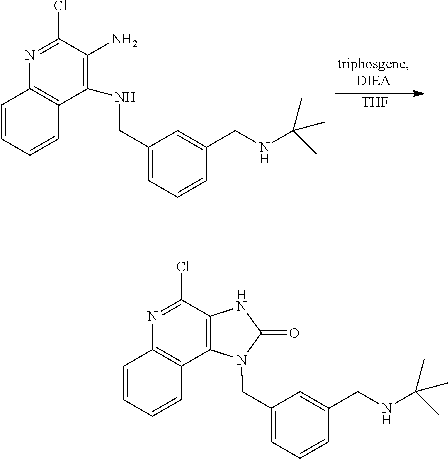 [SMILES: CC(C)(C)NCc1cccc(CNc2c(N)c(Cl)nc3ccccc23)c1 ; CC(C)(C)NCc1cccc(Cn2c(=O)[nH]c3c(Cl)nc4ccccc4c32)c1 ; "THF"]

The starting material N⁴-(3-((tert-butylamino)methyl)benzyl)-2-chloroquinoline-3,4-diamine (800 mg, 2.17 mmol, 1.0 eq) was dissolved in anhydrous tetrahydrofuran (10 mL), and then N,N-diisopropylethylamine (841 mg, 6.51 mmol, 3.0 eq) and triphosgene (643 mg, 2.17 mmol, 1.0 eq) were added. The reaction mixture was stirred at 25° C. for 2 h. After the reaction was completed, as detected by TLC (DCM:MeOH=10:1), the reaction mixture was concentrated, added with water (20 mL) for dilution, and extracted with dichloromethane (20 mL×2). The aqueous phase after the extraction was made basic with saturated sodium bicarbonate and extracted with dichloromethane (20 mL×2). The combined organic phases were dried over anhydrous sodium sulfate, filtered and concentrated to give a solid, which is the target product (600 mg, 70.0%).

### Step 6: Preparation of 4-amino-1-(3-((tert-butylamino)methyl)benzyl)-1H-imidazo[4,5-c]quinolin-2(3H)-one

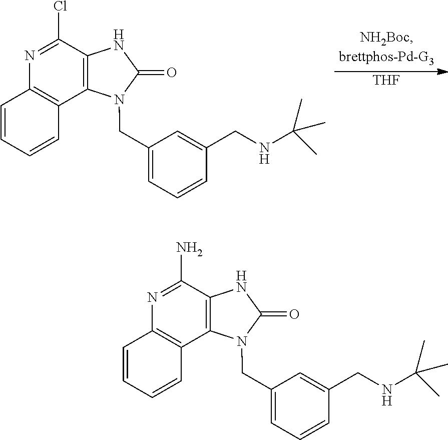 [SMILES: CC(C)(C)NCc1cccc(Cn2c(=O)[nH]c3c(Cl)nc4ccccc4c32)c1 ; CC(C)(C)NCc1cccc(Cn2c(=O)[nH]c3c(N)nc4ccccc4c32)c1 ; "THF"]

The starting material 1-(3-((tert-butylamino)methyl)benzyl)-4-chloro-1H-imidazo[4,5-c]quinolin-2(3H)-one (200 mg, 506 μmol, 1.0 eq) was dissolved in tetrahydrofuran (5 mL). Tert-butyl carbamate (593 mg, 5.06 mmol, 10.0 eq), cesium carbonate (495 mg, 1.52 mmol, 3.0 eq), and brettPhos-Pd-G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (45.9 mg, 50.6 μmol, 0.1 eq) were added sequentially under nitrogen atmosphere. The reaction mixture was stirred at 100° C. for 16 h. After the reaction was completed, as detected by LC-MS, the mixture was filtered, and the filtrate was concentrated to give a crude product, which was separated and purified by Prep-HPLC (0.01% aqueous HCOOH, MeCN) to give the target compound (53.0 mg, 26.5%). LC-MS: [M+H]⁺=376.41; ¹H NMR (400 MHz, DMSO-d₆): δ 7.73 (d, J=8.0 Hz, 1H), 7.73 (d, J=8.4 Hz, 1H), 7.32-7.29 (m, 1H), 7.26 (s, 1H), 7.21 (d, J=4.8 Hz, 2H), 7.05-7.00 (m, 2H), 6.35 (s, 2H), 5.43 (s, 2H), 3.58 (s, 2H), 1.01 (s, 9H).

### Example 19: Preparation of 4-amino-1-(3-(piperazin-1-ylmethyl)benzyl)-1H-imidazo[4,5-c]quinolin-2(3H)-one

### Step 1: Preparation of tert-butyl 4-(3-cyanobenzyl)piperazine-1-carboxylate

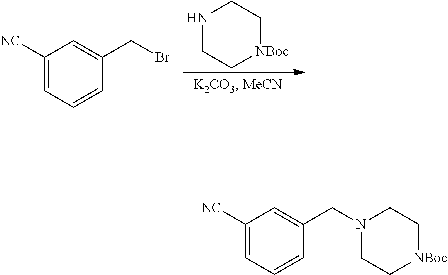

The starting materials 3-(bromomethyl)benzonitrile (5.00 g, 25.5 mmol, 1.0 eq) and tert-butyl piperazine-1-carboxylate (5.68 g, 25.5 mmol, 1.0 eq) were dissolved in acetonitrile (60 mL), and then potassium carbonate (10.6 g, 76.5 mmol, 3.0 eq) was added. The reaction was stirred at 25° C. for 2 h. After the reaction was completed, as detected by TLC (DCM:MeOH=20:1, 254 nm), the reaction mixture was filtered. The filter cake was washed with ethyl acetate. The filtrate was concentrated to remove the acetonitrile. Water (50 mL) was added to the residue. The mixture was extracted with ethyl acetate (100 mL×3). The combined organic phases were dried over anhydrous sodium sulfate, filtered and concentrated to give a crude product, which was separated and purified by flash chromatography (DCM:MeOH=100:0 to 10:1) to give the target compound (7.10 g, 92.4%). LC-MS: [M+H]⁺=302.21.

### Step 2: Preparation of tert-butyl 4-(3-(aminomethyl)benzyl)piperazine-1-carboxylate

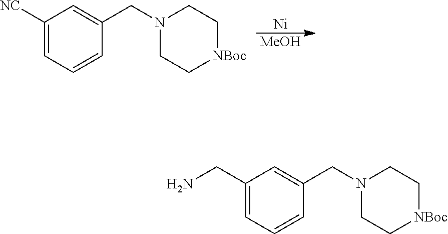

The starting material tert-butyl 4-(3-cyanobenzyl)piperazine-1-carboxylate (3.00 g, 9.95 mmol, 1.0 eq) was dissolved in methanol (50 mL). Raney nickel (853 mg) was added. The reaction mixture was purged with nitrogen 3 times and purged with hydrogen 3 times. The reaction was stirred at 25° C. for 4 h under hydrogen atmosphere (15 Psi). After the reaction was completed, as detected by LC-MS, the mixture was filtered. The filter cake was washed with methanol. The filtrate was concentrated to give the target compound (2.50 g, 82.2%). which was used directly in the next step. LC-MS: [M+H]⁺=306.31; ¹H NMR (400 MHz, CDCl₃): δ 7.30-7.28 (m, 2H), 7.21-7.18 (m, 2H), 3.86 (s, 2H), 3.50 (s, 2H), 3.43-3.41 (m, 4H), 2.39-2.37 (m, 4H), 1.45 (s, 9H).

### Step 3: Preparation of tert-butyl 4-(3-(((2-chloro-3-nitroquinolin-4-yl)amino)methyl)benzyl)piperazine-1-carboxylate

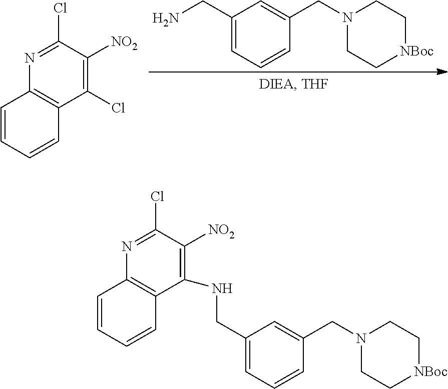 [SMILES: O=[N+]([O-])c1c(Cl)nc2ccccc2c1Cl ; CC(C)(C)OC(=O)N1CCN(Cc2cccc(CN)c2)CC1 ; CC(C)(C)OC(=O)N1CCN(Cc2cccc(CNc3c([N+](=O)[O-])c(Cl)nc4ccccc34)c2)CC1]

The starting material tert-butyl 4-(3-(aminomethyl)benzyl)piperazine-1-carboxylate (1.26 g, 4.11 mmol, 1.0 eq) was dissolved in tetrahydrofuran (15 mL), and then N,N-diisopropylethylamine (1.60 g, 12.3 mmol, 3.0 eq) and 2,4-dichloro-3-nitroquinoline (1.00 g, 4.11 mmol, 1.0 eq) were added. The mixture was stirred at 25° C. for 4 h. After the reaction was completed, as detected by LC-MS, the reaction mixture was filtered. The filter cake was washed with tetrahydrofuran. The filtrate was concentrated. Water (10 mL) was added to the residue. The mixture was extracted with ethyl acetate (20 mL×3). The combined organic phases were washed with saturated brine (30 mL), dried over anhydrous sodium sulfate, filtered and concentrated to give a crude product, which was separated and purified by flash chromatography (PE:EA=100:0 to 10:1) to give the target compound (1.60 g, 76.0%). LC-MS: [M+H]⁺=512.31; ¹H NMR (400 MHz, CDCl₃): δ 7.95 (d, J=7.6 Hz, 1H), 7.86 (d, J=8.0 Hz, 1H), 7.77-7.75 (m, 1H), 7.51-7.49 (m, 1H), 7.37-7.33 (m, 3H), 7.26-7.19 (m, 1H), 5.99 (brs, 1H), 4.59 (d, J=4.8 Hz, 2H), 3.52 (s, 2H), 3.44-3.41 (m, 4H), 2.40-2.37 (m, 4H), 1.46 (s, 9H).

### Step 4: Preparation of tert-butyl 4-(3-(((3-amino-2-chloroquinolin-4-yl)amino)methyl)benzyl)piperazine-1-carboxylate

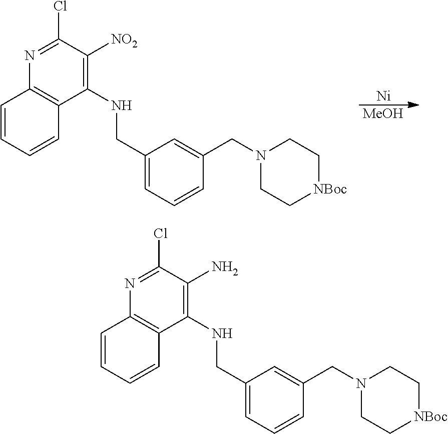

Tert-butyl 4-(3-(((2-chloro-3-nitroquinolin-4-yl)amino)methyl)benzyl)piperazine-1-carboxylate (1.6 g, 3.13 mmol, 1.0 eq) was dissolved in methanol (15 mL). Raney nickel (268 mg) was added. The reaction mixture was purged with nitrogen 3 times and purged with hydrogen 3 times. The reaction was stirred at 25° C. for 4 h under hydrogen atmosphere (15 Psi). After the reaction was completed, as detected by LC-MS, the mixture was filtered. The filter cake was washed with methanol. The filtrate was concentrated to give the target compound (1.20 g, 79.7%). which was used directly in the next step. LC-MS: [M+H]⁺=482.10.

### Step 5: Preparation of tert-butyl 4-(3-((4-chloro-2-oxo-2,3-dihydro-1H-imidazo[4,5-c]quinolin-1-yl)methyl)benzyl)piperazine-1-carboxylate

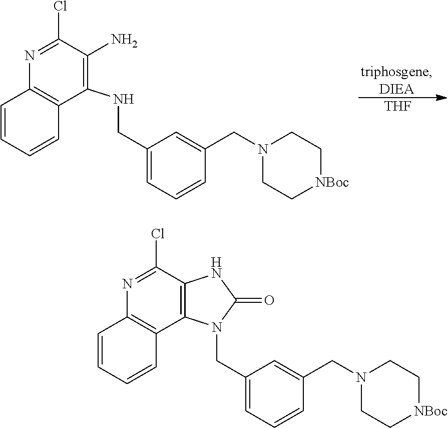

Tert-butyl 4-(3-(((3-amino-2-chloroquinolin-4-yl)amino)methyl)benzyl)piperazine-1-carboxylate (1.00 g, 2.07 mmol, 1.0 eq) was dissolved in tetrahydrofuran (15 mL). N,N-diisopropylethylamine (804 mg, 6.22 mmol, 3.0 eq) and triphosgene (1.00 g, 3.37 mmol, 1.62 eq) were added. The reaction was stirred at 25° C. for 4 h. After the reaction was completed, as detected by LC-MS, the mixture was filtered. The filter cake was washed with tetrahydrofuran. After the filtrate was concentrated, water (10 mL) was added to the residue. The mixture was extracted with ethyl acetate (15 mL×3). The combined organic phases were washed with saturated brine (20 mL), dried over anhydrous sodium sulfate, filtered and concentrated to give a crude product, which was separated and purified by flash chromatography (DCM:MeOH=100:0 to 10:1) to give the target compound (520 mg, 49.3%). LC-MS: [M+H]⁺=508.21.

### Step 6: Preparation of tert-butyl 4-[[3-[(4-amino-2-oxo-3H-imidazo[4,5-c]quinolin-1-yl)methyl]phenyl]methyl]piperazine-1-carboxylate

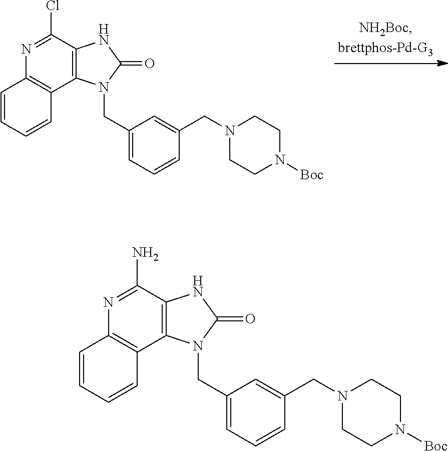 [SMILES: CC(C)(C)OC(=O)N1CCN(Cc2cccc(Cn3c(=O)[nH]c4c(Cl)nc5ccccc5c43)c2)CC1 ; CC(C)(C)OC(=O)N1CCN(Cc2cccc(Cn3c(=O)[nH]c4c(N)nc5ccccc5c43)c2)CC1]

The starting materials tert-butyl 4-(3-((4-chloro-2-oxo-2,3-dihydro-1H-imidazo[4,5-c]quinolin-1-yl)methyl)benzyl)piperazine-1-carboxylate (450 mg, 886 μmol, 1.0 eq) and tert-butyl carbamate (1.04 g, 8.86 mmol, 10.0 eq) were dissolved in tetrahydrofuran (10 mL). Cesium carbonate (866 mg, 2.66 mmol, 3.0 eq) and brettPhos-Pd-G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (80.3 mg, 88.6 μmol, 0.1 eq) were added under nitrogen atmosphere. The reaction was stirred at 100° C. for 4 h. After the reaction was completed, as detected by LC-MS, the reaction mixture was filtered. The filter cake was washed with tetrahydrofuran. The filtrate was concentrated to give a crude product. The target compound (102 mg, 23.57%) was obtained. LC-MS: [M+H]⁺=489.21.

### Step 7: Preparation of 4-amino-1-(3-(piperazin-1-ylmethyl)benzyl)-1H-imidazo[4,5-c]quinolin-2(3H)-one

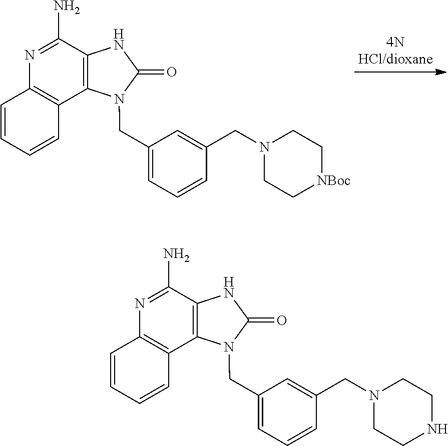 [SMILES: CC(C)(C)OC(=O)N1CCN(Cc2cccc(Cn3c(=O)[nH]c4c(N)nc5ccccc5c43)c2)CC1 ; Nc1nc2ccccc2c2c1[nH]c(=O)n2Cc1cccc(CN2CCNCC2)c1]

The starting material tert-butyl 4-[[3-[(4-amino-2-oxo-3H-imidazo[4,5-c]quinolin-1-yl)methyl]phenyl]methyl]piperazine-1-carboxylate (100 mg, 205 μmol, 1.0 eq) was dissolved in dichloromethane (5 mL). A solution of hydrochloric acid in dioxane (4 mL) was added at 25° C. The reaction mixture was stirred at 25° C. for 4 h. After the reaction was completed, as detected by LC-MS, the reaction mixture was concentrated to give a crude product, which was separated and purified by Prep-HPLC (0.01% aqueous HCOOH, MeCN) to give the target compound (1.20 mg, 1.51%). LC-MS: [M+H]⁺=389.31; ¹H NMR (400 MHz, Methanol-d₄): δ 8.40 (brs, 1H), 7.75 (d, J=8.0 Hz, 1H), 7.63 (d, J=8.4 Hz, 1H), 7.42-7.40 (m, 1H), 7.36-7.34 (m, 1H), 7.29-7.28 (m, 1H), 7.23-7.21 (m, 1H), 7.14-7.10 (m, 2H), 5.59 (s, 2H), 3.50 (s, 2H), 2.97-2.95 (m, 4H), 2.46 (brs, 4H).

### Example 20: Preparation of 4-amino-1-(3-(pyrrolidin-1-yl)benzyl)-1H-imidazo[4,5-c]quinolin-2(3H)-one

### Step 1: Preparation of 3-(pyrrolidin-1-yl)benzonitrile

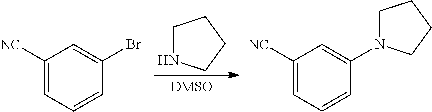

The starting material 3-bromobenzonitrile (5.00 g, 41.3 mmol, 1.0 eq) was dissolved in dimethyl sulfoxide (50 mL). Tetrahydropyrrole (13.5 g, 190 mmol, 4.6 eq) was added. The reaction mixture was stirred at 100° C. for 16 h. After the reaction was completed, as detected by LC-MS, water (100 mL) was added to the reaction mixture. The mixture was extracted with ethyl acetate (100 mL×3). The combined organic phases were washed with saturated brine, dried over anhydrous sodium sulfate, filtered and concentrated to give a crude product, which was separated and purified by flash chromatography (PE:EtOAc=1:0 to 9:1) to give the target compound (5.30 g, 70.8%). LC-MS: [M+H]⁺=173.21.

### Step 2: Preparation of (3-(pyrrolidin-1-yl)phenyl)methylamine

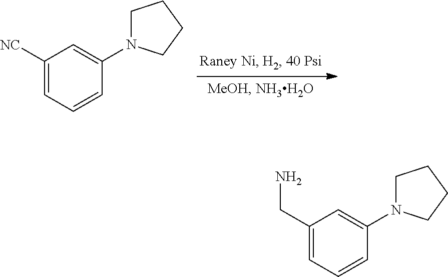 [SMILES: N#Cc1cccc(N2CCCC2)c1 ; NCc1cccc(N2CCCC2)c1]

The starting material 3-(pyrrolidin-1-yl)benzonitrile (5.30 g, 30.8 mmol, 1.0 eq) was dissolved in methanol (50 mL) and aqueous ammonia (10 mL). Raney nickel (900 mg) was added. The reaction mixture was purged with nitrogen 3 times and purged with hydrogen 3 times. The reaction mixture was stirred at 25° C. for 3 h under hydrogen atmosphere (40 Psi). After the reaction was completed, as detected by TLC (PE/EA=10:1), the reaction mixture was filtered, and the filtrate was concentrated to give the target compound (4.90 g, 85.8%), which was used directly in the next step. LC-MS: [M+H]⁺=177.11.

### Step 3: Preparation of 2-chloro-3-nitro-N-(3-(pyrrolidin-1-yl)benzyl)quinoline-4-amine

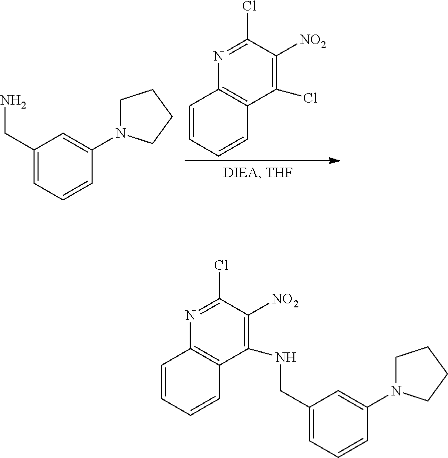

The starting material 2,4-dichloro-3-nitroquinoline (1.00 g, 4.11 mmol, 1.0 eq) was dissolved in tetrahydrofuran (20 mL). (3-(pyrrolidin-1-yl)phenyl)methylamine (724 mg, 4.11 mmol, 4.0 eq) was added. The reaction mixture was stirred at 25° C. for 16 h. After the reaction was completed, as detected by LC-MS, water (50 mL) was added to the reaction mixture. The mixture was extracted with ethyl acetate (50 mL×3). The combined organic phases were washed with saturated brine (50 mL), dried over anhydrous sodium sulfate, filtered and concentrated to give a crude product, which was separated and purified by flash chromatography (PE:EA=1:0 to 1:1) to give the target compound (1.29 g, 77.9%). LC-MS: [M+H]⁺=383.11.

### Step 4: Preparation of 2-chloro-N⁴-(3-(pyrrolidin-1-yl)benzyl)quinolin-3,4-diamine

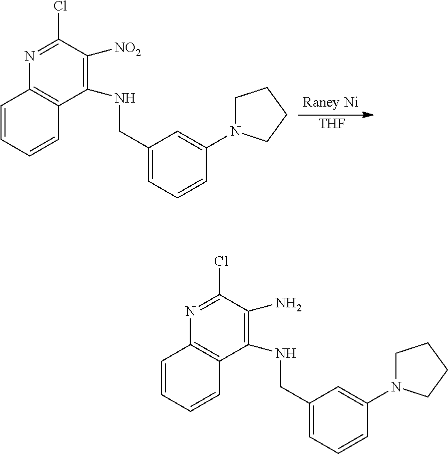 [SMILES: O=[N+]([O-])c1c(Cl)nc2ccccc2c1NCc1cccc(N2CCCC2)c1 ; Nc1c(Cl)nc2ccccc2c1NCc1cccc(N2CCCC2)c1 ; "THF"]

The starting material 2-chloro-3-nitro-N-(3-(pyrrolidin-1-yl)benzyl)quinolin-4-amine (1.19 g, 3.11 mmol, 1.0 eq) was dissolved in tetrahydrofuran (20 mL). Raney nickel (266 mg, 3.11 mmol, 1.0 eq) was added. The reaction mixture was purged with nitrogen 3 times, purged with hydrogen 3 times and stirred at 25° C. for 4 h under hydrogen atmosphere (15 Psi). After the reaction was completed, as detected by LC-MS, the reaction mixture was filtered, and the filtrate was concentrated to give a crude product (730 mg, 66.56%), which was used directly in the next step. LC-MS: [M+H]⁺=353.21; ¹H NMR (400 MHz, DMSO-d₆): δ 7.92 (d, J=8.0 Hz, 1H), 7.73 (d, J=7.6 Hz, 1H), 7.49-7.42 (m, 2H), 7.24-7.20 (m, 1H), 6.68 (d, J=7.2 Hz, 1H), 6.52 (brs, 2H), 4.09 (brs, 2H), 3.26-3.23 (m, 4H), 2.02-1.98 (m, 4H).

### Step 5: Preparation of 4-chloro-1-(3-(pyrrolidin-1-yl)benzyl)-1H-imidazo[4,5-c]quinolin-2(3H)-one

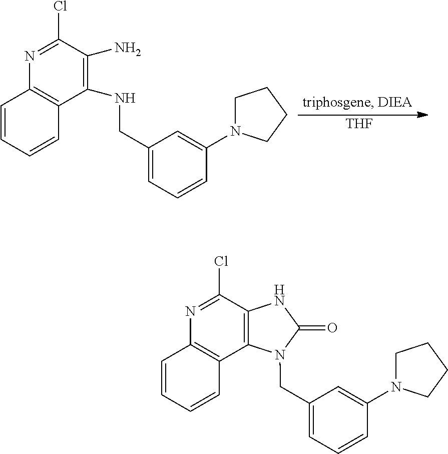 [SMILES: Nc1c(Cl)nc2ccccc2c1NCc1cccc(N2CCCC2)c1 ; O=c1[nH]c2c(Cl)nc3ccccc3c2n1Cc1cccc(N2CCCC2)c1 ; "THF"]

The starting material 2-chloro-N⁴-(3-(pyrrolidin-1-yl)benzyl)quinolin-3,4-diamine (720 mg, 2.04 mmol, 1.0 eq) was dissolved in anhydrous tetrahydrofuran (20 mL), and then N,N-diisopropylethylamine (791 mg, 6.12 mmol, 3.0 eq) and triphosgene (960 mg, 3.24 mmol, 1.6 eq) were added. The reaction mixture was stirred at 25° C. for 4 h. After the reaction was completed, as detected by LC-MS, water (15 mL) was added. The mixture was extracted with ethyl acetate (20 mL×3). The combined organic phases were washed with saturated brine (40 mL), dried over anhydrous sodium sulfate, filtered and concentrated to give the target compound (260 mg, 33.6%), which was used directly in the next step. LC-MS: [M+H]⁺=379.21.

### Step 6: Preparation of 4-amino-1-(3-(pyrrolidin-1-yl)benzyl)-1H-imidazo[4,5-c]quinolin-2(3H)-one

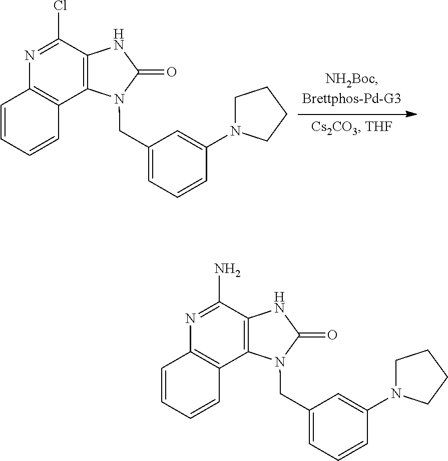 [SMILES: O=c1[nH]c2c(Cl)nc3ccccc3c2n1Cc1cccc(N2CCCC2)c1 ; Nc1nc2ccccc2c2c1[nH]c(=O)n2Cc1cccc(N2CCCC2)c1]

The starting material 4-chloro-1-(3-(pyrrolidin-1-yl)benzyl)-1H-imidazo[4,5-c]quinolin-2(3H)-one (200 mg, 528 μmol, 1.0 eq) was dissolved in tetrahydrofuran (4 mL). Tert-butyl carbamate (618 mg, 5.28 mmol, 10 eq), cesium carbonate (516 mg, 1.58 mmol, 3.0 eq), and brettphos-Pd-G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (47.9 mg, 52.8 μmol, 0.1 eq) were added sequentially under nitrogen atmosphere. The reaction mixture was stirred at 100° C. for 4 h. After the reaction was completed, as detected by LC-MS, the reaction mixture was filtered and the filtrate was concentrated to give a crude product, which was separated and purified by Prep-HPLC (0.1% aqueous TFA solution, ACN) to give the target compound (21.2 mg, 10.8%). LC-MS: [M+H]⁺=360.31; ¹H NMR (400 MHz, DMSO-d₆): δ 11.59 (s, 1H), 8.18 (brs 2H), 8.00 (d, J=8.8 Hz, 1H), 7.75-7.65 (m, 2H), 7.42-7.40 (m, 1H), 7.08-7.04 (m, 1H), 6.50 (s, 1H), 6.44-6.34 (m, 2H), 5.40 (s, 2H), 3.15 (s, 4H), 1.91 (s, 4H).

### Example 21: Preparation of 4-amino-1-(3-(pyridin-3-yl)benzyl)-1H-imidazo[4,5-c]quinolin-2(3H)-one

### Step 1: Preparation of 3-(pyridin-3-yl)benzonitrile

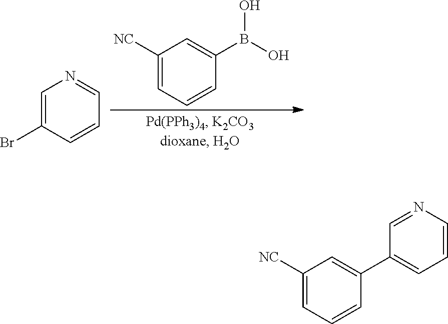 [SMILES: Brc1cccnc1 ; N#Cc1cccc(B(O)O)c1 ; N#Cc1cccc(-c2cccnc2)c1]

The starting material 3-bromopyridine (5.00 g, 31.7 mmol, 1.0 eq) was dissolved in 1,4-dioxane (50 mL) and water (50 mL). (3-cyanophenyl)boronic acid (5.58 g, 38.0 mmol, 1.2 eq), potassium carbonate (8.75 g, 63.3 mmol, 2.0 eq), and tetrakis(triphenylphosphine)palladium(0) (768 mg, 665 μmol, 0.02 eq) were added at 25° C. The reaction mixture was stirred at 90° C. for 12 h under nitrogen atmosphere. After the reaction was completed, as detected by LC-MS, the reaction mixture was added with water (50 mL) for dilution. The mixture was extracted with ethyl acetate (50 mL×3). The combined organic phases were dried over anhydrous sodium sulfate, filtered and concentrated to give a black crude product, which was separated and purified by flash chromatography (PE:EA=100:20 to 100:40) to give the target compound (5.20 g, 91.2%). LC-MS: [M+H]⁺=181.0.

### Step 2: Preparation of (3-(pyridin-3-yl)phenyl)methylamine

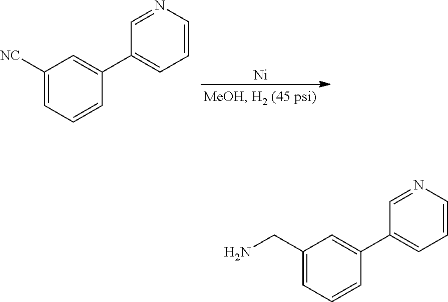

The starting material 3-(pyridin-3-yl)benzonitrile (2.00 g, 11.1 mmol, 1.0 eq) was dissolved in methanol (20 mL). Aqueous ammonia (5 mL) and Raney nickel (951 mg) were added. The reaction mixture was purged with nitrogen 3 times and purged with hydrogen 3 times. The reaction was stirred at 25° C. for 4 h under hydrogen atmosphere (45 Psi). After the reaction was completed, as detected by TLC (PE:EA=3:1), the mixture was filtered. The filter cake was washed with methanol. The filtrate was concentrated to give the target compound (2.00 g, 97.8%), which was used directly in the next step.

### Step 3: Preparation of 2-chloro-3-nitro-N-(3-(pyridin-3-yl)benzyl)quinoline-4-amine

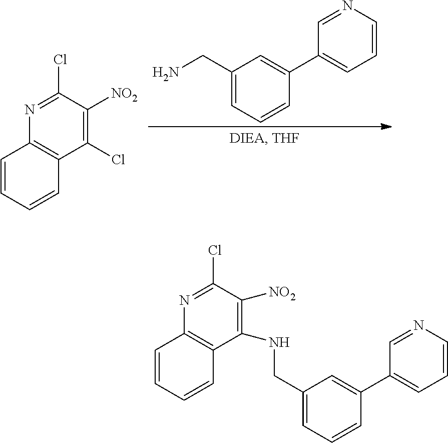 [SMILES: O=[N+]([O-])c1c(Cl)nc2ccccc2c1Cl ; NCc1cccc(-c2cccnc2)c1 ; O=[N+]([O-])c1c(Cl)nc2ccccc2c1NCc1cccc(-c2cccnc2)c1]

The starting material 2,4-dichloro-3-nitroquinoline (1.00 g, 4.11 mmol, 1.0 eq) was dissolved in tetrahydrofuran (15 mL). (3-(pyridin-3-yl)phenyl)methylamine (910 mg, 4.94 mmol, 1.2 eq) and N,N-diisopropylethylamine (1.60 g, 12.3 mmol, 3.0 eq) were added. The reaction mixture was stirred at 25° C. for 2 h. After the reaction was completed, as detected by LC-MS, the reaction mixture was concentrated to remove the tetrahydrofuran to give a crude product, and then water (20 mL) was added to the reaction mixture. The mixture was extracted with dichloromethane (20 mL×3). The combined organic phases were dried over anhydrous sodium sulfate, filtered and concentrated to give a crude product, which was separated and purified by flash chromatography (DCM:MeOH=80:1 to 50:1) to give the target compound (1.30 g, 80.8%). LC-MS: [M+H]⁺=391.1.

### Step 4: Preparation of 2-chloro-N⁴-(3-(pyridin-3-yl)benzyl)quinolin-3,4-diamine

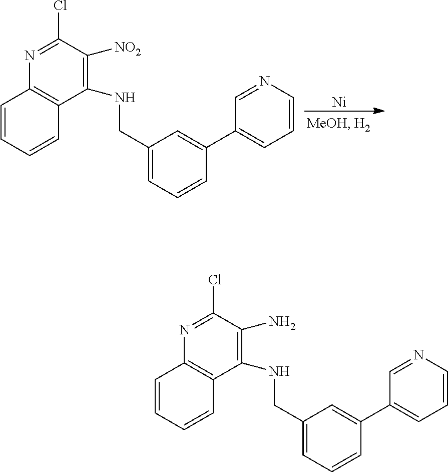

The starting material 2-chloro-3-nitro-N-(3-(pyridin-3-yl)benzyl)quinolin-4-amine (1.30 g, 3.33 mmol, 1.0 eq) was dissolved in methanol (15 mL). After Raney nickel (285 mg, 3.33 mmol, 1.0 eq) was added, the reaction mixture was purged with hydrogen several times. The reaction was stirred at 25° C. for 2 h under hydrogen atmosphere (15 Psi). After the reaction was completed, as detected by LC-MS, the mixture was filtered. The filter cake was washed with methanol and concentrated to remove methanol to give a crude product, which was separated and purified by flash chromatography (DCM:MeOH=80:0 to 50:1) to give the target compound (830 mg, 69.2%). LC-MS: [M+H]⁺=361.2.

### Step 5: Preparation of 4-chloro-1-(3-(pyridin-3-yl)benzyl)-1H-imidazo[4,5-c]quinolin-2(3H)-one

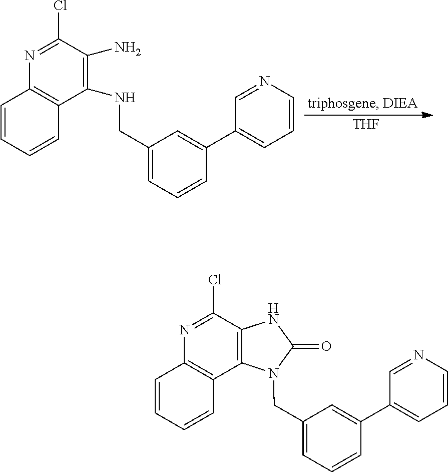

The starting material 2-chloro-N⁴-(3-(pyridin-3-yl)benzyl)quinolin-3,4-diamine (830 mg, 2.30 mmol, 1.0 eq) was dissolved in tetrahydrofuran (10 mL). N,N-diisopropylethylamine (892 mg, 6.90 mmol, 3.0 eq). Triphosgene (580 mg, 1.95 mmol, 0.85 eq) was added. The reaction mixture was stirred at 25° C. for 16 h. After the reaction was completed, as detected by LC-MS, the reaction mixture was concentrated and was added with water (10 mL) for dilution. The mixture was extracted with ethyl acetate (10 mL×3). The organic phase was discarded. The aqueous phase was adjusted to pH 9 with saturated aqueous sodium bicarbonate solution and extracted with dichloromethane (10 mL×3). The organic phases were dried over anhydrous sodium sulfate, filtered and concentrated to give a crude product (420 mg, 47.2%), which was used directly in the next step without purification. LC-MS: [M+H]⁺=387.0.

### Step 6: Preparation of 4-amino-1-(3-(pyridin-3-yl)benzyl)-1H-imidazo[4,5-c]quinolin-2(3H)-one

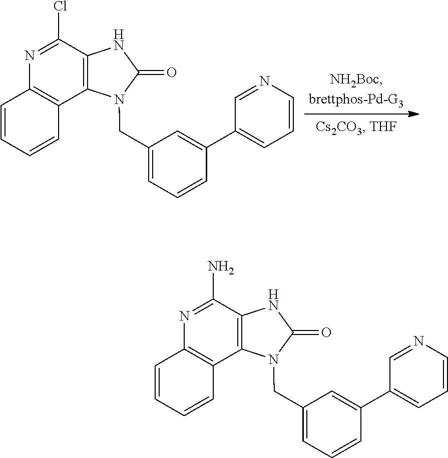 [SMILES: O=c1[nH]c2c(Cl)nc3ccccc3c2n1Cc1cccc(-c2cccnc2)c1 ; Nc1nc2ccccc2c2c1[nH]c(=O)n2Cc1cccc(-c2cccnc2)c1]

The starting material 4-chloro-1-(3-(pyridin-3-yl)benzyl)-1H-imidazo[4,5-c]quinolin-2(3H)-one (200 mg, 517 μmol, 1.0 eq) was dissolved in tetrahydrofuran (5 mL). Tert-butyl carbamate (606 mg, 5.17 mmol, 10 eq), cesium carbonate (505 mg, 1.55 mmol, 3.0 eq), and brettPhos-Pd-G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (46.9 mg, 51.7 μmol, 0.1 eq) were added respectively under nitrogen atmosphere. The reaction mixture was stirred at 100° C. for 5 h. After the reaction was completed, as detected by LC-MS, and after the starting material was completely reacted, the reaction mixture was filtered to remove the cesium carbonate. After the reaction mixture was added with water (10 mL) for dilution, the pH was adjusted to 5 with 1 N diluted hydrochloric acid. The mixture was extracted with ethyl acetate (10 mL×3). The organic phase was discarded. The aqueous phase was adjusted to pH 9 with saturated aqueous sodium bicarbonate solution and extracted with dichloromethane (10 mL×3). The organic phases were dried over anhydrous sodium sulfate, filtered and concentrated to give a crude product, which was separated and purified by Prep-HPLC (0.05% aqueous HCl solution, MeCN) to give the target compound (11.0 mg, 5.79%). LC-MS: [M+H]⁺=368.3; 1H NMR (400 MHz, MeOD-d₄): δ 9.15 (brs, 1H), 8.85 (br, J=8.8 Hz, 2H), 8.13-8.12 (m, 1H), 8.05 (d, J=8.0 Hz, 1H), 7.85 (s, 1H), 7.78-7.75 (m, 2H), 7.70-7.66 (m, 1H), 7.59 (t, J=7.8 Hz, 1H), 7.48-7.42 (m, 2H), 5.76 (s, 2H).

### Example 22: Preparation of 4-amino-1-(3-(cyclopentylamino)benzyl)-1H-imidazo[4,5-c]quinolin-2(3H)-one

### Step 1: Preparation of tert-butyl 3-(cyclopentylamino)benzylcarbamate

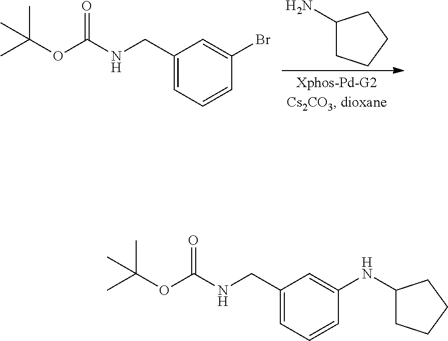 [SMILES: CC(C)(C)OC(=O)NCc1cccc(Br)c1 ; NC1CCCC1 ; CC(C)(C)OC(=O)NCc1cccc(NC2CCCC2)c1]

The starting materials tert-butyl 3-bromobenzylcarbamate (2.50 g, 8.74 mmol, 1.0 eq) and cyclopentylamine (1.49 g, 17.4 mmol, 2.0 eq) were dissolved in dioxane (50 mL). Cesium carbonate (8.54 g, 26.2 mmol, 3.0 eq) and Xphos-Pd-G2 (chloro(2-dicyclohexylphosphino-2,4,6-triisopropyl-1,1-biphenyl)[2-(2-amino-1,1-biphenyl)]palladium(II)) (1.03 g, 1.31 mmol, 0.15 eq) were added sequentially. The reaction mixture was stirred at 100° C. for 16 h. After the reaction was completed, as detected by LC-MS, the reaction mixture was filtered, and the filtrate was concentrated to give a crude product, which was separated and purified by flash chromatography (PE:EA=1:0 to 8:1) to give the target compound (2.00 g, 78.8%). LC-MS: [M+H]⁺=291.2.

### Step 2: Preparation of 3-(aminomethyl)-N-cyclopentylaniline

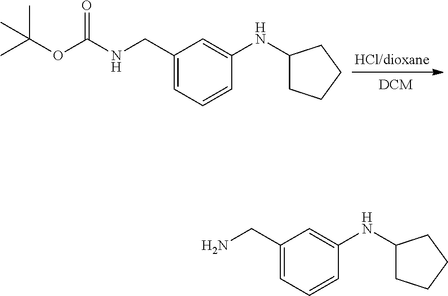 [SMILES: CC(C)(C)OC(=O)NCc1cccc(NC2CCCC2)c1 ; NCc1cccc(NC2CCCC2)c1]

The starting material tert-butyl 3-(cyclopentylamino)benzylcarbamate (2.00 g, 6.89 mmol, 1.0 eq) was dissolved in dichloromethane (20 mL). A solution of hydrochloric acid in dioxane (4 mL) was added. The reaction mixture was stirred at 25° C. for 2 h. After the reaction was completed, as detected by TLC (PE:EA=8:1), the reaction mixture was concentrated to give the target compound (2.04 g, crude product), which was used directly in the next step.

### Step 3: Preparation of 2-chloro-N-(3-(cyclopentylamino)benzyl)-3-nitroquinoline-4-amine

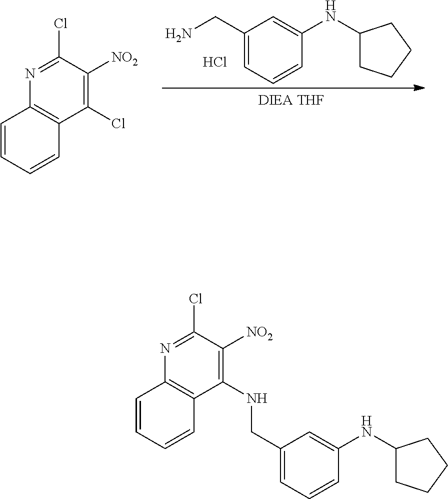 [SMILES: O=[N+]([O-])c1c(Cl)nc2ccccc2c1Cl ; NCc1cccc(NC2CCCC2)c1 ; Cl ; O=[N+]([O-])c1c(Cl)nc2ccccc2c1NCc1cccc(NC2CCCC2)c1]

The starting material 2,4-dichloro-3-nitroquinoline (1.00 g, 4.11 mmol, 1.0 eq) was dissolved in anhydrous tetrahydrofuran (15 mL), and then N,N-diisopropylethylamine (2.66 g, 20.6 mmol, 5.0 eq) and 3-(aminomethyl)-N-cyclopentylaniline hydrochloride (783 mg, 4.11 mmol, 1.0 eq) were added. The reaction mixture was stirred at 25° C. for 2 h. After the reaction was completed, as detected by LC-MS, water (15 mL) was added. The mixture was extracted with ethyl acetate (20 mL×3). The combined organic phases were washed with saturated brine (30 mL), dried over anhydrous sodium sulfate, filtered and concentrated to give the target compound (1.33 g, 81.5%). LC-MS: [M+H]⁺=397.2.

### Step 4: Preparation of 2-chloro-N⁴-(3-(cyclopentylamino)benzyl)quinolin-3,4-diamine

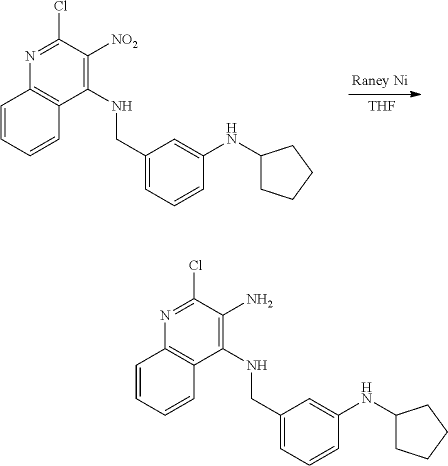 [SMILES: O=[N+]([O-])c1c(Cl)nc2ccccc2c1NCc1cccc(NC2CCCC2)c1 ; Nc1c(Cl)nc2ccccc2c1NCc1cccc(NC2CCCC2)c1 ; "THF"]

The starting material 2-chloro-N-(3-(cyclopentylamino)benzyl)-3-nitroquinolin-4-amine (1.33 g, 3.35 mmol, 1.0 eq) was dissolved in tetrahydrofuran (15 mL). Raney nickel (287 mg) was added. The reaction mixture was purged with nitrogen 3 times, purged with hydrogen 3 times and stirred at 25° C. for 4 h under hydrogen atmosphere (15 Psi). After the reaction was completed, as detected by LC-MS, the reaction mixture was filtered, and the filtrate was concentrated to give a solid. The solid was separated and purified by flash chromatography (DCM:MeOH=1:0 to 10:1) to give the target compound (520 mg, 42.3%). LC-MS: [M+H]⁺=367.0.

### Step 5: Preparation of 4-chloro-1-(3-(cyclopentylamino)benzyl)-1H-imidazo[4,5-c]quinolin-2(3H)-one

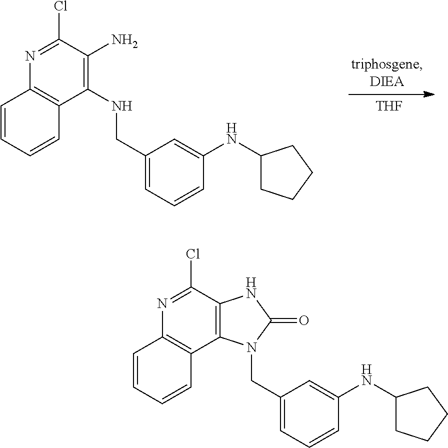 [SMILES: Nc1c(Cl)nc2ccccc2c1NCc1cccc(NC2CCCC2)c1 ; O=c1[nH]c2c(Cl)nc3ccccc3c2n1Cc1cccc(NC2CCCC2)c1 ; "THF"]

The starting material 2-chloro-N⁴-(3-(cyclopentylamino)benzyl)quinolin-3,4-diamine (400 mg, 1.09 mmol, 1.0 eq) was dissolved in anhydrous tetrahydrofuran (8 mL), and then N,N-diisopropylethylamine (423 mg, 3.27 mmol, 3.0 eq) and triphosgene (380 mg, 1.28 mmol, 1.2 eq) were added. The reaction mixture was stirred at 25° C. for 4 h. After the reaction was completed, as detected by LC-MS, water (10 mL) was added. The pH was adjusted to 4 with 1 N diluted hydrochloric acid. The mixture was extracted with ethyl acetate (10 mL×3). The aqueous phase was adjusted to pH 8 with saturated sodium bicarbonate solution and extracted with dichloromethane (15 mL×3). The combined organic phases were dried over anhydrous sodium sulfate, filtered and concentrated to give the target compound (240 mg, 56.03%). LC-MS: [M+H]+=393.2.

### Step 6: Preparation of 4-amino-1-(3-(cyclopentylamino)benzyl)-1H-imidazo[4,5-c]quinolin-2(3H)-one

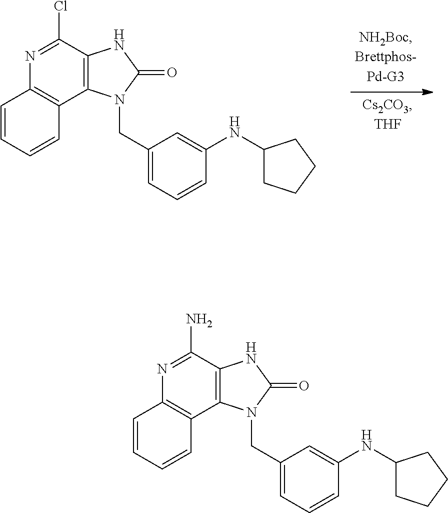 [SMILES: O=c1[nH]c2c(Cl)nc3ccccc3c2n1Cc1cccc(NC2CCCC2)c1 ; Nc1nc2ccccc2c2c1[nH]c(=O)n2Cc1cccc(NC2CCCC2)c1]

The starting material 4-chloro-1-(3-(cyclopentylamino)benzyl)-1H-imidazo[4,5-c]quinolin-2(3H)-one (200 mg, 509 μmol, 1.0 eq) was dissolved in tetrahydrofuran (4 mL). Tert-butyl carbamate (596 mg, 5.09 mmol, 10.0 eq), cesium carbonate (498 mg, 1.53 mmol, 3.0 eq), and brettphos-Pd-G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (4.61 mg, 5.09 μmol, 0.1 eq) were added sequentially under nitrogen atmosphere. The reaction mixture was stirred at 100° C. for 4 h. After the reaction was completed, as detected by LC-MS, the reaction mixture was filtered and the filtrate was concentrated to give a crude product, which was separated and purified by Prep-HPLC (0.225% aqueous HCOOH, ACN) to give the target compound (31.4 mg, 16.5%). LC-MS: [M+H]⁺=374.4; ¹H NMR (400 MHz, DMSO-d₆): δ 10.99 (brs, 1H), 7.77 (d, J=8.0 Hz, 1H), 7.53 (d, J=8.0 Hz, 1H), 7.35-7.31 (m, 1H), 7.07-6.98 (m, 2H), 6.40-6.35 (m, 5H), 5.58 (brs, 1H), 5.31 (s, 2H), 3.52 (brs, 1H), 1.81-1.79 (m, 2H), 1.61-1.59 (m, 2H), 1.48-1.46 (m, 2H), 1.37-1.32 (m, 2H).

### Example 23: Preparation of 4-amino-1-(4-chloro-3-(pyrrolidin-1-ylmethyl)benzyl)-1H-imidazo[4,5-c]quinolin-2(3H)-one

### Step 1: Preparation of (2-chloro-5-iodophenyl)methanol

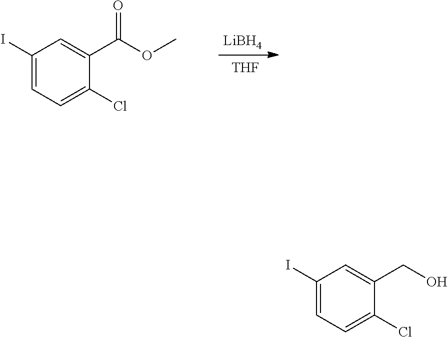

The starting material methyl 2-chloro-5-iodobenzoate (18.0 g, 60.7 mmol, 1.0 eq) was dissolved in anhydrous tetrahydrofuran (200 mL). Methanol (3.89 g, 121 mmol, 4.91 mL, 2.0 eq) and lithium borohydride (5.29 g, 242 mmol, 4.0 eq) were added. The reaction mixture was stirred at 25° C. for 2 h. After the reaction was completed, as detected by TLC (DCM:MeOH=10:1), the reaction mixture was quenched with 1 M HCl (150 mL), and then water (150 mL) was added. The mixture was extracted with ethyl acetate (300 mL). The organic phase was washed with saturated brine (200 mL). The combined organic phases were dried over anhydrous sodium sulfate, filtered and concentrated to give the target compound (8.00 g, 49.1%). which was used directly in the next step.

### Step 2: Preparation of 1-chloro-2-(chloromethyl)-4-iodobenzene

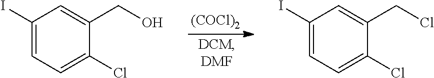

The starting material (2-chloro-5-iodophenyl)methanol (8.00 g, 29.8 mmol, 1.0 eq) was dissolved in anhydrous dichloromethane (50 mL). Oxalyl chloride (11.4 g, 89.4 mmol, 7.82 mL, 3.0 eq) was added. N,N-dimethylformamide (0.5 mL) was added dropwise. The reaction mixture was stirred at 25° C. for 2 h. After the reaction was completed, as detected by TLC (PE:EA=10:1), the reaction mixture was concentrated to give the target compound (8.00 g, 93.6%). which was used directly in the next step.

### Step 3: Preparation of 1-(2-chloro-5-iodobenzyl)pyrrolidine

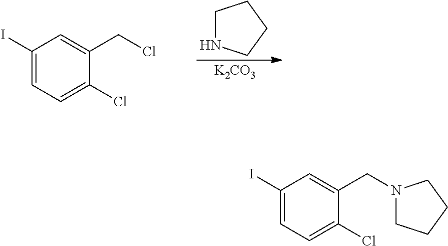

The starting material 1-chloro-2-(chloromethyl)-4-iodobenzene (8.00 g, 27.9 mmol, 1.0 eq) was dissolved in acetonitrile (50 mL). Potassium carbonate (11.6 g, 83.7 mmol, 3.0 eq) and tetrahydropyrrole (3.97 g, 55.8 mmol, 2.0 eq) were added. The reaction mixture was stirred at 25° C. for 18 h. After the reaction was completed, as detected by LC-MS, water (150 mL) was added. The mixture was extracted with ethyl acetate (150 mL×2). The combined organic phases were washed with saturated brine (200 mL), dried over anhydrous sodium sulfate, filtered and concentrated to give a crude product, which was separated and purified by flash chromatography (PE:EA=100:0 to 88:12) to give the target compound (2.10 g, 23.4%). LC-MS: [M+H]⁺=321.7.

### Step 4: Preparation of 4-chloro-3-(pyrrolidin-1-ylmethyl)benzonitrile

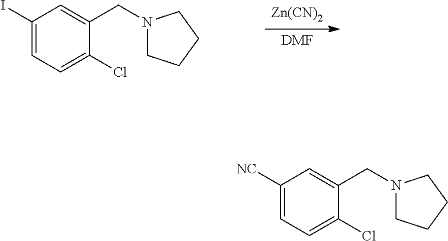

The starting material 1-(2-chloro-5-iodobenzyl)pyrrolidine (1.90 g, 5.91 mmol, 1.00 eq) was dissolved in N,N-dimethylformamide (20 mL). Zinc cyanide (693 mg, 5.91 mmol, 1.0 eq) and tetrakis(triphenylphosphine)palladium(0) (682 mg, 590 μmol, 0.1 eq) were added under nitrogen atmosphere. The reaction mixture was stirred at 90° C. for 12 h. After the reaction was completed, as detected by LC-MS, a mixture of aqueous ammonia and water (25% NH₃·H₂O:H₂O=1:1, 30 mL) was added to the reaction mixture. The mixture was extracted with ethyl acetate (30 mL×2). The combined organic phases were washed with saturated brine (25 mL×2), dried over anhydrous sodium sulfate, filtered and concentrated to give a crude product, which was separated and purified by flash chromatography (DCM:MeOH=100:0 to 93:7) to give the target compound (1.00 mg, 76.7%). LC-MS: [M+H]⁺=221.3.

### Step 5: Preparation of (4-chloro-3-(pyrrolidin-1-ylmethyl)phenyl)methyl amine

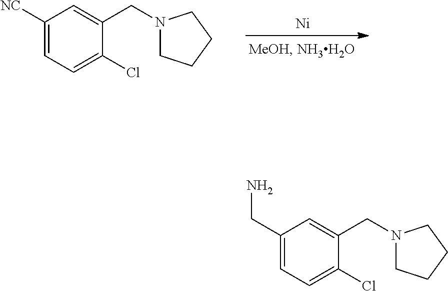

The starting material 4-chloro-3-(pyrrolidin-1-ylmethyl)benzonitrile (1.00 g, 4.53 mmol, 1.0 eq) was dissolved in methanol (10 mL). Aqueous ammonia (0.6 mL) and Raney nickel (1.16 g) were added. The reaction mixture was purged with nitrogen 3 times, purged with hydrogen 3 times and stirred at 25° C. for 1 h under hydrogen atmosphere (15 Psi). After the reaction was completed, as detected by LC-MS, the reaction mixture was filtered. The filter cake was washed with methanol. The filtrate was concentrated to give a crude product, which was separated and purified by flash chromatography (DCM:MeOH=100:0 to 95:5) to give the target compound (920 mg, 90.4%). LC-MS: [M+H]⁺=224.2.

### Step 6: Preparation of 2-chloro-N-(4-chloro-3-(pyrrolidin-1-ylmethyl)benzyl)-3-nitroquinoline-4-amine

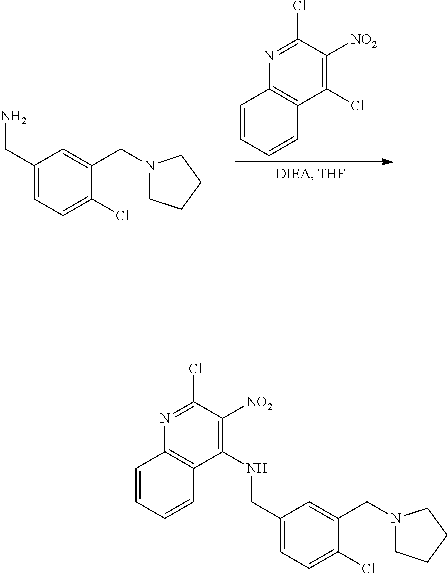 [SMILES: NCc1ccc(Cl)c(CN2CCCC2)c1 ; O=[N+]([O-])c1c(Cl)nc2ccccc2c1Cl ; O=[N+]([O-])c1c(Cl)nc2ccccc2c1NCc1ccc(Cl)c(CN2CCCC2)c1]

The starting material 2,4-dichloro-3-nitroquinoline (900 mg, 3.70 mmol, 1.0 eq) was dissolved in anhydrous tetrahydrofuran (10 mL). N,N-diisopropylethylamine (1.44 g, 11.1 mmol, 3.0 eq) and (4-chloro-3-(pyrrolidin-1-ylmethyl)phenyl)methylamine (873 mg, 3.89 mmol, 1.1 eq) were added. The reaction mixture was stirred at 25° C. for 5 h. After the reaction was completed, as detected by LC-MS, water (15 mL) was added. The mixture was extracted with ethyl acetate (30 mL×2). The combined organic phases were washed with saturated brine (15 mL), dried over anhydrous sodium sulfate, filtered and concentrated to give a crude product, which was separated and purified by flash chromatography (DCM:MeOH=100:0 to 95:5) to give the target compound (1.02 g, 63.9%). LC-MS: [M+H]⁺=431.3.

### Step 7: Preparation of 2-chloro-N⁴-(4-chloro-3-(pyrrolidin-1-ylmethyl)benzyl)quinolin-3,4-diamine

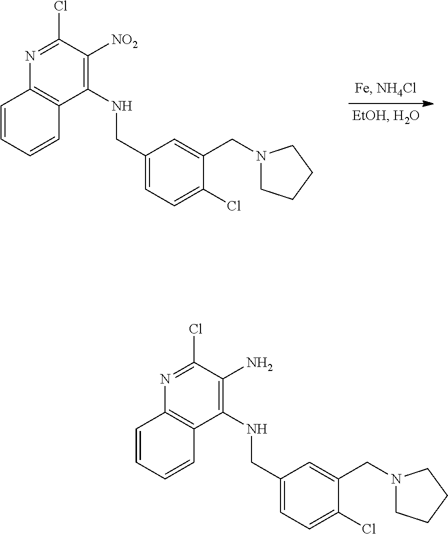 [SMILES: O=[N+]([O-])c1c(Cl)nc2ccccc2c1NCc1ccc(Cl)c(CN2CCCC2)c1 ; Nc1c(Cl)nc2ccccc2c1NCc1ccc(Cl)c(CN2CCCC2)c1]

The starting material 2-chloro-N-(4-chloro-3-(pyrrolidin-1-ylmethyl)benzyl)-3-nitroquinolin-4-amine (750 mg, 1.74 mmol, 1.0 eq) was dissolved in ethanol (15 mL) and water (3 mL). Ammonium chloride (372 mg, 6.96 mmol, 4.0 eq) and iron powder (388 mg, 6.96 mmol, 4.0 eq) were added. The reaction mixture was stirred at 50° C. for 2 h. After the reaction was completed, as detected by LC-MS, the reaction mixture was filtered and concentrated to give a crude product, which was separated and purified by flash chromatography (DCM:MeOH=100:0 to 93:7) to give the target compound (390 mg, 55.9%). LC-MS: [M+H]⁺=400.1.

### Step 8: Preparation of 4-chloro-1-(4-chloro-3-(pyrrolidin-1-ylmethyl)benzyl)-1H-imidazo[4,5-c]quinolin-2(3H)-one

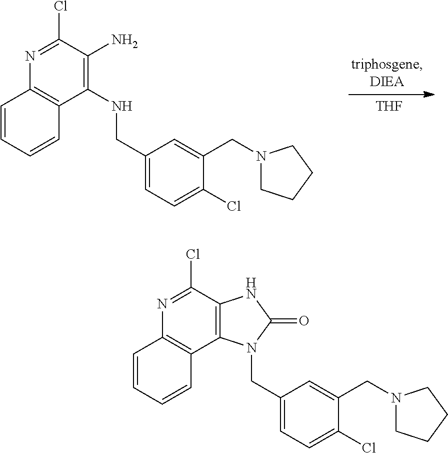 [SMILES: Nc1c(Cl)nc2ccccc2c1NCc1ccc(Cl)c(CN2CCCC2)c1 ; O=c1[nH]c2c(Cl)nc3ccccc3c2n1Cc1ccc(Cl)c(CN2CCCC2)c1 ; "THF"]

The starting material 2-chloro-N⁴-(4-chloro-3-(pyrrolidin-1-ylmethyl)benzyl)quinolin-3,4-diamine (250 mg, 622 μmol, 1.0 eq) was dissolved in anhydrous tetrahydrofuran (5 mL). N,N-diisopropylethylamine (241 mg, 1.87 mmol, 3.0 eq) and triphosgene (184 mg, 623 μmol, 1.0 eq) were added. The reaction mixture was stirred at 25° C. for 2 h. After the reaction was completed, as detected by LC-MS, the solvent was removed by rotary evaporation. Ethyl acetate (10 mL) and water (10 mL) were added. The organic phase was discarded. The aqueous phase was adjusted to pH 8 with saturated sodium bicarbonate solution and extracted with ethyl acetate (10 mL×2). The combined organic phases were dried over anhydrous sodium sulfate, filtered and concentrated to give the target compound (120 mg, 45.1%), which was used directly in the next step. LC-MS: [M+H]⁺=427.3.

### Step 9: Preparation of 4-amino-1-(4-chloro-3-(pyrrolidin-1-ylmethyl)benzyl)-1H-imidazo[4,5-c]quinolin-2(3H)-one

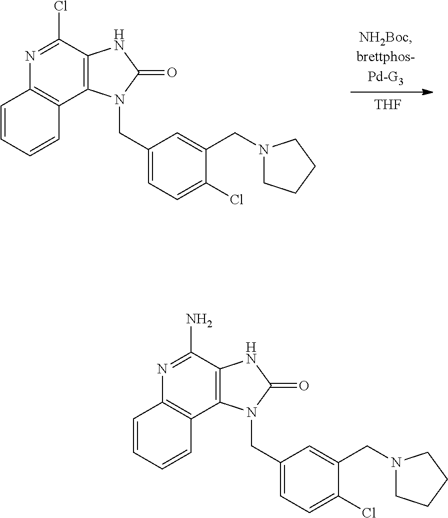 [SMILES: O=c1[nH]c2c(Cl)nc3ccccc3c2n1Cc1ccc(Cl)c(CN2CCCC2)c1 ; Nc1nc2ccccc2c2c1[nH]c(=O)n2Cc1ccc(Cl)c(CN2CCCC2)c1 ; "THF"]

The starting material 4-chloro-1-(4-chloro-3-(pyrrolidin-1-ylmethyl)benzyl)-1H-imidazo[4,5-c]quinolin-2(3H)-one (100 mg, 234 μmol, 1.0 eq) was dissolved in anhydrous tetrahydrofuran (2 mL). Tert-butyl carbamate (274 mg, 2.34 mmol, 10 eq), cesium carbonate (228 mg, 702 μmol, 3.0 eq), and brettphos-Pd-G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (21.2 mg, 23.4 μmol, 0.1 eq) were added sequentially under nitrogen atmosphere. The reaction mixture was stirred at 100° C. for 3 h. After the reaction was completed, as detected by LC-MS, water (4 mL) was added to the mixture. The mixture was extracted with ethyl acetate (4 mL×2). The combined organic phases were washed with saturated brine (2 mL), dried over anhydrous sodium sulfate, filtered and concentrated to give a solid. The solid was separated and purified by Prep-HPLC (0.01% aqueous HCOOH, MeCN) to give the target compound (31.0 mg, 32.5%). LC-MS: [M+H]⁺=408.3; ¹H NMR (400 MHz, MeOD-d₄): δ 7.87-7.64 (m, 2H), 7.59-7.26 (m, 4H), 7.19-7.15 (m, 1H), 5.95-5.62 (m, 2H), 4.28-4.12 (m, 2H), 2.97-2.81 (m, 4H), 1.90-1.83 (m, 4H).

### Example 24: Preparation of 4-amino-1-(3-(pyridin-2-yl)benzyl)-1H-imidazo[4,5-c]quinolin-2(3H)-one

### Step 1: Preparation of 3-(pyridin-2-yl)benzonitrile

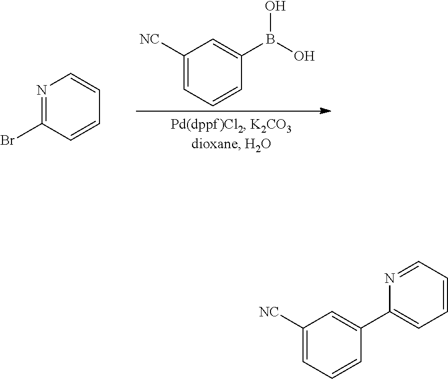 [SMILES: Brc1ccccn1 ; N#Cc1cccc(B(O)O)c1 ; N#Cc1cccc(-c2ccccn2)c1]

The starting material 2-bromopyridine (5.50 g, 34.8 mmol, 1.0 eq) was dissolved in dioxane (90 mL) and water (30 mL). (3-cyanophenyl)boronic acid (6.14 g, 41.8 mmol, 1.2 eq), potassium carbonate (14.4 g, 104 mmol, 3.0 eq), and [1,1-bis(di-tert-butylphosphino)ferrocene]dichloropalladium(II) (1.27 g, 1.74 mmol, 0.05 eq) were added. The reaction mixture was stirred at 100° C. for 2 h under nitrogen atmosphere. After the reaction was completed, as detected by TLC (PE:EA=3:1), water (100 mL) was added to the reaction mixture. The mixture was extracted with ethyl acetate (30 mL×3). The combined organic phases were dried over anhydrous sodium sulfate, filtered and concentrated to give a crude product, which was separated and purified by flash chromatography (PE:EA=10:1 to 3:1) to give the target compound (6.20 g, 98.8%).

### Step 2: Preparation of (3-(pyridin-2-yl)phenyl)methylamine

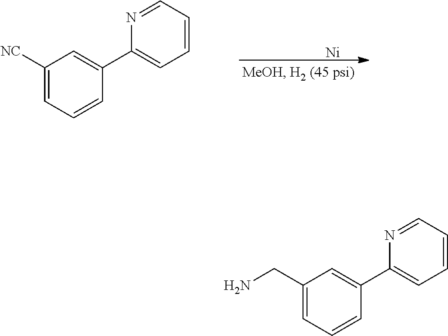

The starting material 3-(pyridin-2-yl)benzonitrile (2.00 g, 11.1 mmol, 1.0 eq) was dissolved in methanol (20 mL). Aqueous ammonia (4 mL) and Raney nickel (951 mg) were added. The reaction mixture was purged with nitrogen 3 times and purged with hydrogen 3 times. The reaction was stirred at 25° C. for 2 h under hydrogen atmosphere (45 Psi). After the reaction was completed, as detected by TLC (PE:EA=3:1), the mixture was filtered. The filter cake was washed with methanol. After the methanol was removed by concentration, the target compound (2.00 g, 97.8%) was obtained. The crude product was used directly in the next step.

### Step 3: Preparation of 2-chloro-3-nitro-N-(3-(pyridin-2-yl)benzyl)quinoline-4-amine

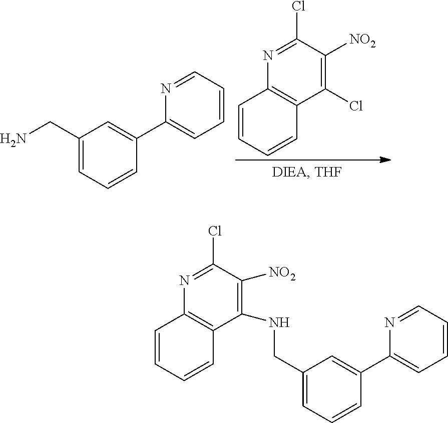

The starting material (3-(pyridin-2-yl)phenyl)methylamine (1.00 g, 4.11 mmol, 1 eq) was dissolved in tetrahydrofuran (15 mL). 2,4-dichloro-3-nitroquinoline (910 mg, 4.94 mmol, 1.2 eq) and DIEA (1.60 g, 12.3 mmol, 3 eq) were added. The reaction mixture was stirred at 25° C. for 3 h under nitrogen atmosphere. After the reaction was completed, as detected by TLC (PE:EA=10:1), the reaction mixture was concentrated to remove the tetrahydrofuran. Water (20 mL) was added to the reaction mixture. The mixture was extracted with dichloromethane (20 mL×3). The combined organic phases were dried over anhydrous sodium sulfate, filtered and concentrated to give a yellow crude product, which was separated and purified by flash chromatography (DCM:MeOH=100:0 to 80:1) to give the target compound (1.03 g, 64.1%).

### Step 4: Preparation of 2-chloro-N⁴-(3-(pyridin-2-yl)benzyl)quinolin-3,4-diamine

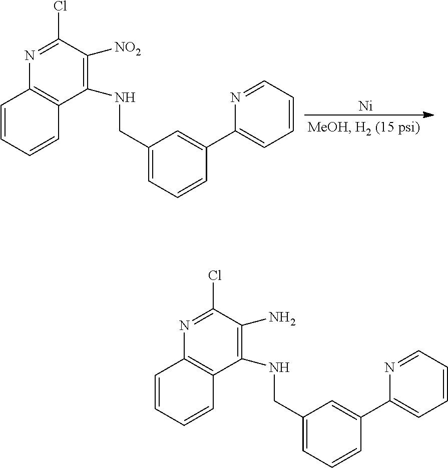

The starting material 2-chloro-3-nitro-N-(3-(pyridin-2-yl)benzyl)quinolin-4-amine (1.03 g, 2.64 mmol, 1.0 eq) was dissolved in methanol (10 mL). Raney nickel (226 mg, 2.64 mmol, 1.0 eq) was added. The reaction mixture was purged with nitrogen 3 times and purged with hydrogen 3 times, and then the reaction was stirred at 25° C. for 2 h under hydrogen atmosphere (15 Psi). After the reaction was completed, as detected by LC-MS, the mixture was filtered. The filter cake was washed with methanol (20 mL). The filtrate was concentrated to give the target compound (550 mg, 57.8%), which was used directly in the next step. LC-MS: [M+H]⁺=361.2.

### Step 5: Preparation of 4-chloro-1-(3-(pyridin-2-yl)benzyl)-1H-imidazo[4,5-c]quinolin-2(3H)-one

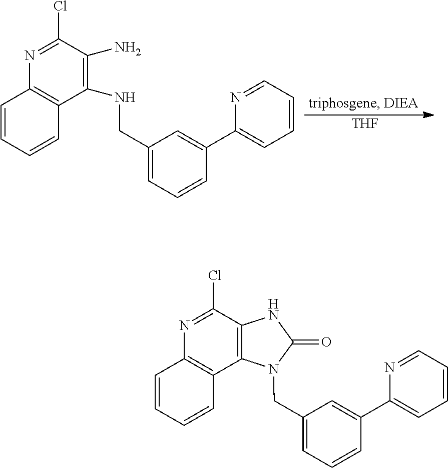 [SMILES: Nc1c(Cl)nc2ccccc2c1NCc1cccc(-c2ccccn2)c1 ; "triphosgene, DIEA" ; O=c1[nH]c2c(Cl)nc3ccccc3c2n1Cc1cccc(-c2ccccn2)c1 ; "THF"]

The starting material 2-chloro-N⁴-(3-(pyridin-2-yl)benzyl)quinolin-3,4-diamine (500 mg, 1.39 mmol, 1 eq) was dissolved in tetrahydrofuran (10 mL). Triphosgene (430 mg, 1.45 mmol, 1.05 eq) and N,N-diisopropylethylamine (537 mg, 4.16 mmol, 3.0 eq) were added. The reaction mixture was stirred at 25° C. for 2 h under nitrogen atmosphere. After the reaction was completed, as detected by LC-MS, the reaction mixture was concentrated to remove the THF. Water (10 mL) was added. The mixture was extracted with ethyl acetate (10 mL×3). The aqueous phase was adjusted to pH 9 with saturated sodium bicarbonate solution and extracted with dichloromethane (10 mL×3). The combined organic phases were dried over anhydrous sodium sulfate, filtered and concentrated to give a yellow crude product, which was added with ethyl acetate (15 mL) and recrystallized at 25° C. to give the target compound (420 mg, 78.4%). LC-MS: [M+H]⁺=387.2.

### Step 6: Preparation of 4-amino-1-(3-(pyridin-2-yl)benzyl)-1H-imidazo[4,5-c]quinolin-2(3H)-one

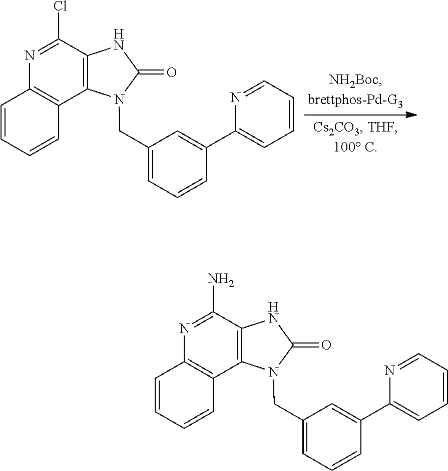 [SMILES: O=c1[nH]c2c(Cl)nc3ccccc3c2n1Cc1cccc(-c2ccccn2)c1 ; Nc1nc2ccccc2c2c1[nH]c(=O)n2Cc1cccc(-c2ccccn2)c1]

The starting material 4-chloro-1-(3-(pyridin-2-yl)benzyl)-1H-imidazo[4,5-c]quinolin-2(3H)-one (100 mg, 259 μcool, 1.0 eq) was dissolved in tetrahydrofuran (3 mL). Tert-butyl carbamate (303 mg, 2.59 mmol, 10 eq), brettPhos-Pd-G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (23.4 mg, 25.9 μcool, 0.1 eq), and cesium carbonate (253 mg, 776 μcool, 3.0 eq) were added. The reaction mixture was stirred at 100° C. for 16 h under nitrogen atmosphere. After the reaction was completed, as detected by LC-MS, the reaction mixture was concentrated to remove the tetrahydrofuran to give a yellow crude product, which was separated and purified by Prep-HPLC (0.05% aqueous HCl solution, MeOH) to give the target compound (35.0 mg, 36.9%). LC-MS: [M+H]⁺=368.3; ¹H NMR (400 MHz, DMSO-d₆): δ 8.68-8.65 (m, 1H), 8.39-8.31 (m, 2H), 8.09 (s, 1H), 8.01-7.96 (m, 4H), 7.76 (d, J=8.8 Hz, 1H), 7.64 (t, J=6.4 Hz, 1H), 7.40-7.31 (m, 4H), 5.64 (s, 2H).

### Example 25: Preparation of 4-amino-8-methyl-1-(3-(pyrrolidin-1-ylmethyl)benzyl)-1H-imidazo[4,5-c]quinolin-2(3H)-one

### Step 1: Preparation of 6-methyl-3-nitroquinolin-2,4-diol

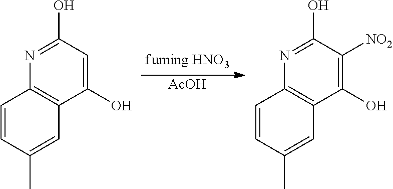 [SMILES: "AcOH" ; Cc1ccc2nc(O)cc(O)c2c1 ; Cc1ccc2nc(O)c([N+](=O)[O-])c(O)c2c1]

The starting material 6-methylquinolin-2,4-diol (3.80 g, 21.7 mmol, 1.0 eq) was dissolved in glacial acetic acid (20 mL), and then fuming nitric acid (22 mL) was added at 0° C. The reaction mixture was stirred at 25° C. for 2 h. After the reaction was completed, as detected by LC-MS, the mixture was added to ice water (100 mL), and a solid precipitated. The mixture was filtered and concentrated by rotary evaporation to give the target compound (3.90 g, 81.6%). LC-MS: [M+H]+=221.1.

### Step 2: Preparation of 2,4-dichloro-6-methyl-3-nitroquinoline

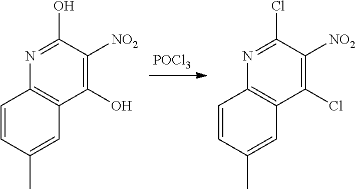

The starting material 6-methyl-3-nitroquinolin-2,4-diol (3.90 g, 17.7 mmol, 1.0 eq) was dissolved in phosphorus oxychloride (30 mL) at 25° C. The reaction was warmed to 40° C. N,N-diisopropylethylamine (6.61 g, 44.3 mmol, 2.5 eq) was added. The mixture was stirred at 60° C. for 3 h. After the reaction was completed, as detected by TLC (PE:EA=5:1), the reaction mixture was slowly added to warm water (100 mL). The mixture was extracted with dichloromethane (100 mL×3). The combined organic phases were washed with saturated brine (200 mL), dried over anhydrous sodium sulfate, filtered and concentrated to give a solid, which was separated and purified by flash chromatography (PE:EA=1:0 to 5:1) to give the target compound (1.90 g, 41.7%).

### Step 3: Preparation of 2-chloro-6-methyl-3-nitro-N-(3-(pyrrolidin-1-ylmethyl)benzyl)quinoline-4-amine

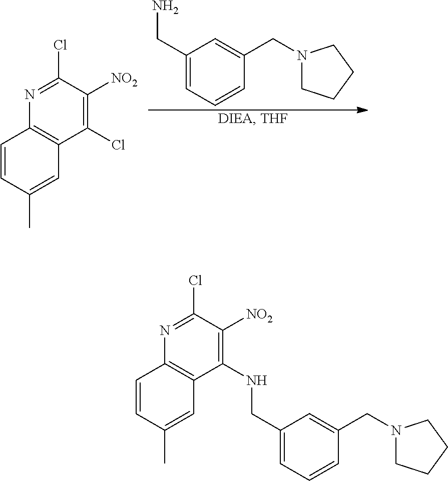

The starting materials 2,4-dichloro-6-methyl-3-nitroquinoline (800 mg, 3.11 mmol, 1.0 eq) and [3-(pyrrolidin-1-ylmethyl)phenyl]methylamine (592 mg, 3.11 mmol, 1.0 eq) were dissolved in anhydrous tetrahydrofuran (6 mL), and then N,N-diisopropylethylamine (1.21 g, 9.34 mmol, 3.0 eq) was added. The reaction mixture was stirred at 25° C. for 12 h. After the reaction was completed, as detected by LC-MS, water (10 mL) was added to the mixture. The mixture was extracted with dichloromethane (10 mL×3). The combined organic phases were washed with saturated brine (20 mL), dried over anhydrous sodium sulfate, filtered and concentrated to give a solid. The reaction mixture was poured into ethyl acetate (10 mL), slurried at 25° C. and filtered to give the target compound (1.10 g, 86.3%). LC-MS: [M+H]⁺=411.2.

### Step 4: Preparation of 2-chloro-6-methyl-N⁴-(3-(pyrrolidin-1-ylmethyl)benzyl)quinolin-3,4-diamine

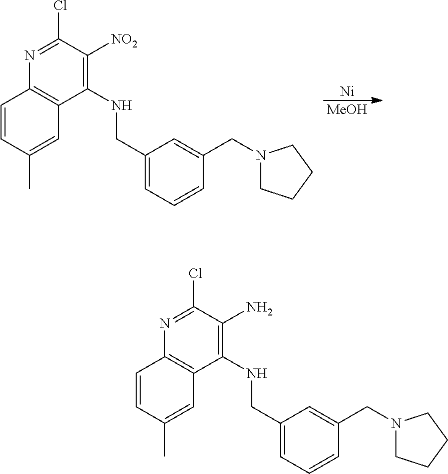

The starting material 2-chloro-6-methyl-3-nitro-N-(3-(pyrrolidin-1-ylmethyl)benzyl)quinolin-4-amine (1.10 g, 2.68 mmol, 1.0 eq) was dissolved in absolute methanol (15 mL). Raney nickel (229.36 mg) was added. The reaction mixture was purged with nitrogen 3 times, purged with hydrogen 3 times and stirred at 25° C. for 4 h under hydrogen atmosphere (15 Psi). After the reaction was completed, as detected by LC-MS, the reaction mixture was filtered. The filter cake was washed with methanol. The filtrate was concentrated to give the target compound (900 mg, 88.3%). LC-MS: [M+H]⁺=381.2.

### Step 5: Preparation of 4-chloro-8-methyl-1-(3-(pyrrolidin-1-ylmethyl)benzyl)-1H-imidazo[4,5-c]quinolin-2(3H)-one

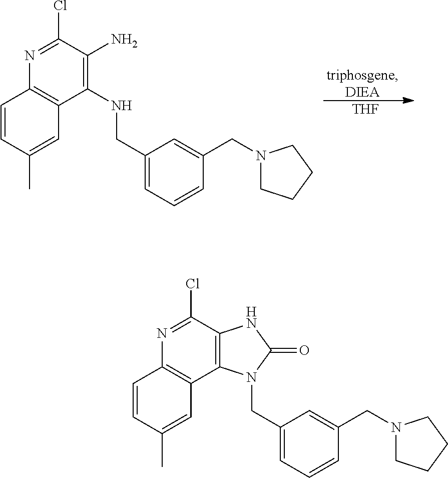 [SMILES: Cc1ccc2nc(Cl)c(N)c(NCc3cccc(CN4CCCC4)c3)c2c1 ; "triphosgene,DIEA" ; Cc1ccc2nc(Cl)c3[nH]c(=O)n(Cc4cccc(CN5CCCC5)c4)c3c2c1 ; "THF"]

The starting material 2-chloro-6-methyl-N⁴-(3-(pyrrolidin-1-ylmethyl)benzyl)quinolin-3,4-diamine (900 mg, 2.36 mmol, 1.0 eq) was dissolved in anhydrous tetrahydrofuran (8 mL), and then N,N-diisopropylethylamine (916 mg, 7.09 mmol, 3.0 eq) and triphosgene (761 mg, 2.56 mmol, 1.1 eq) were added. The reaction mixture was stirred at 25° C. for 4 h. After the reaction was completed, as detected by LC-MS, water (10 mL) was added to the reaction mixture. The mixture was extracted with dichloromethane (15 mL×3). The combined organic phases were washed with saturated brine (20 mL), dried over anhydrous sodium sulfate, filtered and concentrated to give the target compound (720 mg, 74.9%). LC-MS: [M+H]⁺=407.2.

### Step 6: Preparation of 4-amino-8-methyl-1-(3-(pyrrolidin-1-ylmethyl)benzyl)-1H-imidazo[4,5-c]quinolin-2(3H)-one

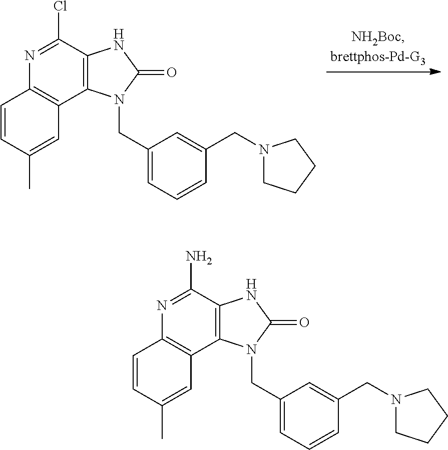 [SMILES: Cc1ccc2nc(Cl)c3[nH]c(=O)n(Cc4cccc(CN5CCCC5)c4)c3c2c1 ; Cc1ccc2nc(N)c3[nH]c(=O)n(Cc4cccc(CN5CCCC5)c4)c3c2c1]

The starting material 4-chloro-8-methyl-1-(3-(pyrrolidin-1-ylmethyl)benzyl)-1H-imidazo[4,5-c]quinolin-2(3H)-one (200 mg, 492 μmol, 1.0 eq) was dissolved in tetrahydrofuran (5 mL). Tert-butyl carbamate (576 mg, 4.92 mmol, 10.0 eq), cesium carbonate (480 mg, 1.47 mmol, 3.0 eq), and brettphos-Pd-G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (44.6 mg, 49.2 μmol, 0.1 eq) were added sequentially under nitrogen atmosphere. The reaction mixture was stirred at 100° C. for 12 h. After the reaction was completed, as detected by LC-MS, the reaction mixture was filtered and the filtrate was concentrated to give a crude product, which was separated and purified by Prep-HPLC (0.225% aqueous HCOOH, ACN) to give the target compound (31.3 mg, 16.4%). LC-MS: [M+H]⁺=388.4; ¹H NMR (400 MHz, DMSO-d₆): δ 7.50 (s, 1H), 7.40 (d, J=8.4 Hz, 1H), 7.26-7.24 (m, 2H), 7.17-7.15 (m, 2H), 7.13-7.09 (m, 1H), 6.23 (s, 2H), 5.43 (s, 2H), 3.54 (s, 2H), 2.33 (brs, 4H), 2.22 (s, 3H), 1.61 (brs, 4H).

### Example 26: Preparation of 4-amino-1-((5-(pyrrolidin-1-ylmethyl)thien-2-yl)methyl)-1H-imidazo[4,5-c]quinolin-2(3H)-one

### Step 1: Preparation of 2-chloro-3-nitro-N-((5-(pyrrolidin-1-ylmethyl)thien-2-yl)methyl)quinoline-4-amine

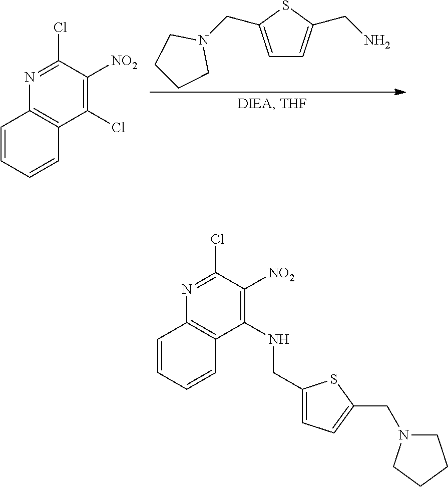

The starting material 2,4-dichloro-3-nitroquinoline (1.00 g, 4.11 mmol, 1.0 eq) was dissolved in anhydrous tetrahydrofuran (15 mL). N,N-diisopropylethylamine (1.60 g, 12.3 mmol, 3.0 eq) and (5-(pyrrolidin-1-ylmethyl)thien-2-yl)methylamine (808 mg, 4.11 mmol, 1.0 eq) were added. The reaction mixture was stirred at 25° C. for 1 h. After the reaction was completed, as detected by LC-MS, water (20 mL) was added to the reaction mixture. The mixture was extracted with ethyl acetate (20 mL×2). The organic phase was washed with saturated brine (30 mL). The combined organic phases were dried over anhydrous sodium sulfate, filtered and concentrated to give a crude product, which was separated and purified by flash chromatography (DCM:MeOH=100:0 to 30:1) to give the target compound (1.20 g, 72.4%). LC-MS: [M+H]⁺=403.0.

### Step 2: Preparation of 2-chloro-N⁴-((5-(pyrrolidin-1-ylmethyl)thien-2-yl)methyl)quinolin-3,4-diamine

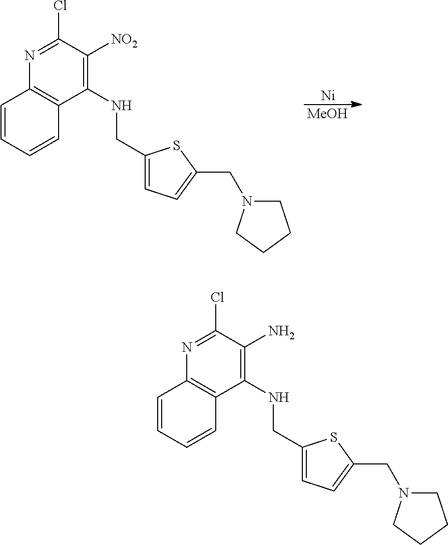

2-chloro-3-nitro-N-((5-(pyrrolidin-1-ylmethyl)thien-2-yl)methyl)quinolin-4-amine (900 mg, 2.23 mmol, 1.0 eq) was dissolved in methanol (20 mL) Raney nickel (191 mg) was added under nitrogen atmosphere. The reaction mixture was purged with nitrogen 3 times and then purged with hydrogen 3 times. The reaction mixture was stirred at 25° C. for 2 h under hydrogen atmosphere (15 Psi). After the reaction was completed, as detected by TLC (DCM:MeOH=20:1), the reaction was filtered. The filtrate was concentrated to give the target compound (820 mg, 98.4%), which was used directly in the next step.

### Step 3: Preparation of 4-chloro-1-((5-(pyrrolidin-1-ylmethyl)thien-2-yl)methyl)-1H-imidazo[4,5-c]quinolin-2(3H)-one

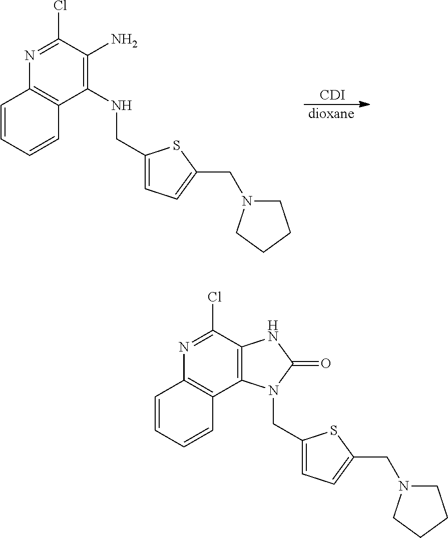 [SMILES: Nc1c(Cl)nc2ccccc2c1NCc1ccc(CN2CCCC2)s1 ; "CDI" ; "dioxane" ; O=c1[nH]c2c(Cl)nc3ccccc3c2n1Cc1ccc(CN2CCCC2)s1]

The starting material 2-chloro-N⁴-((5-(pyrrolidin-1-ylmethyl)thien-2-yl)methyl)quinolin-3,4-diamine (950 mg, 2.55 mmol, 1.0 eq) was dissolved in 1,4-dioxane (20 mL). Bis(1H-imidazol-1-yl)methanone (4.13 g, 25.5 mmol, 10.0 eq) was added. The reaction mixture was stirred at 100° C. for 16 h. After the reaction was completed, as detected by LC-MS, the reaction mixture was concentrated to give a crude product, which was separated and purified by flash chromatography (DCM:MeOH=1:0 to 30:1) to give the target compound (240 mg, 23.6). LC-MS: [M+H]⁺=399.1.

### Step 4: Preparation of 4-amino-1-((5-(pyrrolidin-1-ylmethyl)thien-2-yl)methyl)-1H-imidazo[4,5-c]quinolin-2(3H)-one

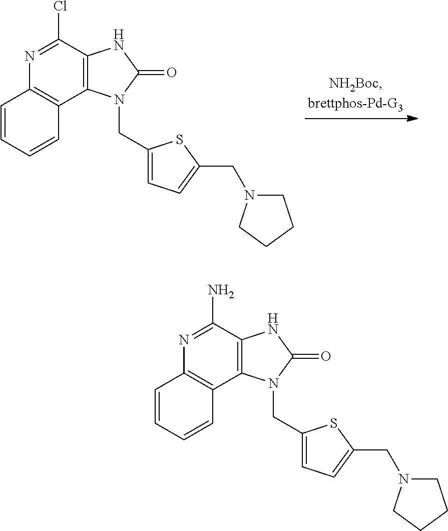 [SMILES: O=c1[nH]c2c(Cl)nc3ccccc3c2n1Cc1ccc(CN2CCCC2)s1 ; Nc1nc2ccccc2c2c1[nH]c(=O)n2Cc1ccc(CN2CCCC2)s1]

4-chloro-1-((5-(pyrrolidin-1-ylmethyl)thien-2-yl)methyl)-1H-imidazo[4,5-c]quinolin-2(3H)-one (200 mg, 501 μmol, 1.0 eq) was dissolved in tetrahydrofuran (4 mL). Tert-butyl carbamate (176 mg, 1.50 mmol, 3.0 eq), cesium carbonate (490 mg, 1.50 mmol, 3.0 eq), and brettphos-Pd-G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (45.5 mg, 50.1 μmol, 0.1 eq) were added respectively under nitrogen atmosphere. The reaction mixture was stirred at 100° C. for 2 h. After the reaction was completed, as detected by LC-MS, the reaction mixture was filtered and concentrated to give a crude product, which was separated and purified by Prep-HPLC (0.225% aqueous HCOOH, MeCN) to give the target compound (15.0 mg, 7.75%). LC-MS: [M+H]⁺=380.3; ¹H NMR: (400 MHz, DMSO-d₆): δ 7.99 (d, J=8.0 Hz, 1H), 7.55 (d, J=8.4 Hz, 1H), 7.39-7.36 (m, 1H), 7.18-7.15 (m, 1H), 6.94 (s, 1H), 6.81 (s, 1H), 6.41 (s, 2H), 5.55 (s, 2H), 3.73 (s, 2H), 2.49 (s, 4H), 1.66 (s, 4H).

### Example 27: Preparation of 4-amino-1-(3-(pyrrolidin-1-ylmethyl)benzyl)-1H-imidazo[4,5-c][1,8]naphthyridine-2(3H)-one

### Step 1: Preparation of 2-chloro-3-nitro-N-(3-(pyrrolidin-1-ylmethyl)benzyl)-1,8-naphthyridine-4-amine

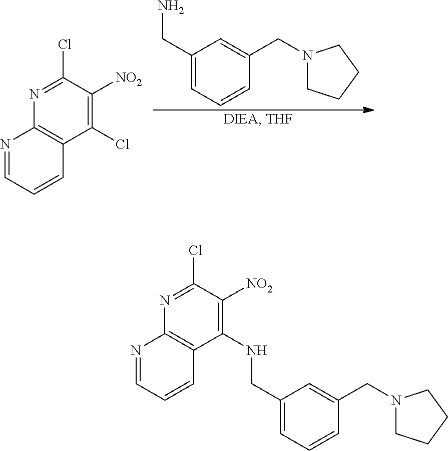

The starting material 2,4-dichloro-3-nitro-1,8-naphthyridine (580 mg, 2.38 mmol, 1.0 eq) was dissolved in anhydrous tetrahydrofuran (6 mL). N,N-diisopropylethylamine (922 mg, 7.13 mmol, 3.0 eq) and (3-(pyrrolidin-1-ylmethyl)phenyl)methylamine (497 mg, 2.61 mmol, 1.1 eq) were added. The reaction mixture was stirred at 25° C. for 8 h. After the reaction was completed, as detected by LC-MS, water (10 mL) was added to the reaction mixture. The mixture was extracted with dichloromethane (10 mL×2). The organic phase was washed with saturated brine (10 mL). The combined organic phases were dried over anhydrous sodium sulfate, filtered and concentrated to give a crude product, which was slurried with ethyl acetate (2 mL) to give the target compound (830 mg, 87.9%). LC-MS: [M+H]⁺=398.1.

### Step 2: Preparation of 2-chloro-N⁴-(3-(pyrrolidin-1-ylmethyl)benzyl)-1,8-naphthyridine-3,4-diamine

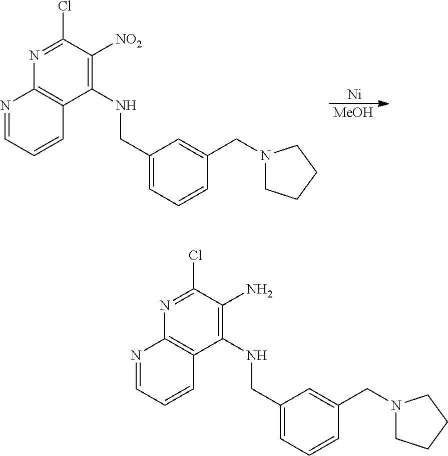

The starting material 2-chloro-3-nitro-N-(3-(pyrrolidin-1-ylmethyl)benzyl)-1,8-naphthyridine-4-amine (650 mg, 1.63 mmol, 1.0 eq) was dissolved in methanol (5 mL) Raney nickel (140 mg) was added under nitrogen atmosphere. The reaction mixture was purged with nitrogen 3 times and then purged with hydrogen 3 times. The reaction mixture was stirred at 25° C. for 4 h under hydrogen atmosphere (15 Psi). After the reaction was completed, as detected by LC-MS, the reaction mixture was filtered, and the filtrate was concentrated to give a crude product, which was separated and purified by flash chromatography (DCM:MeOH=100:0 to 96:4) to give the target compound (426 mg, 70.8%). LC-MS: [M+H]⁺=368.2.

### Step 3: Preparation of 4-chloro-1-(3-(pyrrolidin-1-ylmethyl)benzyl)-1H-imidazo[4,5-c][1,8]naphthyridine-2(3H)-one

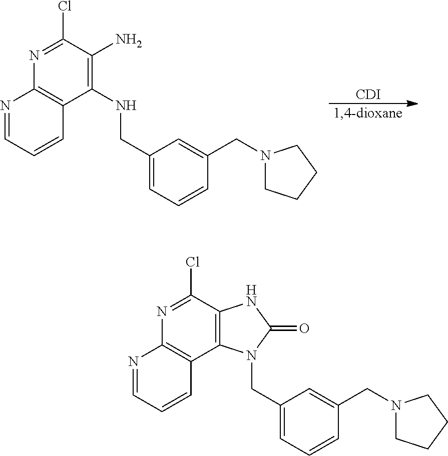 [SMILES: Nc1c(Cl)nc2ncccc2c1NCc1cccc(CN2CCCC2)c1 ; "CDI" ; "1,4-dioxane" ; O=c1[nH]c2c(Cl)nc3ncccc3c2n1Cc1cccc(CN2CCCC2)c1]

The starting material 2-chloro-N-4-(3-(pyrrolidin-1-ylmethyl)benzyl)-1,8-naphthyridine-3,4-diamine (453 mg, 1.23 mmol, 1.0 eq) was dissolved in 1,4-dioxane (5 mL). Bis(1H-imidazol-1-yl)methanone (1.20 g, 7.38 mmol, 6.0 eq) was added. The reaction mixture was stirred at 90° C. for 18 h. After the reaction was completed, as detected by LC-MS, the reaction mixture was added into sodium bicarbonate (10 mL) to quench the reaction. Water (10 mL) was added. The mixture was extracted with dichloromethane (20 mL×2). The combined organic phases were dried over anhydrous sodium sulfate, filtered and concentrated to give a crude product, which was concentrated to give a crude product, which was separated and purified by flash chromatography (DCM:MeOH=100:0 to 96:4) to give the target compound (240 mg, 49.5%). LC-MS: [M+H]⁺=394.3.

### Step 4: Preparation of 4-amino-1-(3-(pyrrolidin-1-ylmethyl)benzyl)-1H-imidazo[4,5-c][1,8]naphthyridine-2(3H)-one

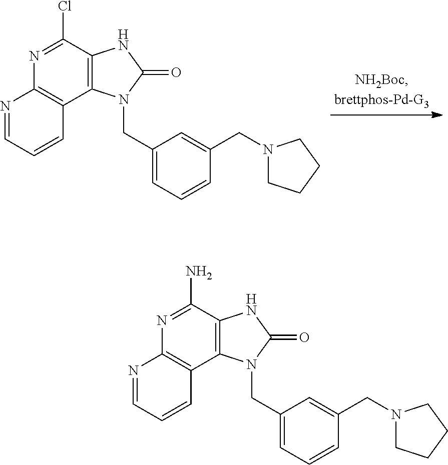 [SMILES: O=c1[nH]c2c(Cl)nc3ncccc3c2n1Cc1cccc(CN2CCCC2)c1 ; Nc1nc2ncccc2c2c1[nH]c(=O)n2Cc1cccc(CN2CCCC2)c1]

The starting material 4-chloro-1-(3-(pyrrolidin-1-ylmethyl)benzyl)-1H-imidazo[4,5-c][1,8]naphthyridine-2(3H)-one (190 mg, 482 mmol, 1.0 eq) was dissolved in tetrahydrofuran (2 mL). Tert-butyl carbamate (565 mg, 4.82 mmol, 10 eq), cesium carbonate (472 mg, 1.45 mmol, 3.0 eq), and brettPhos-Pd-G3 (methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (43.7 mg, 48.2 μmol, 0.1 eq) were added respectively under nitrogen atmosphere. The reaction mixture was stirred at 100° C. for 3 h. After the reaction was completed, as detected by LC-MS, the reaction mixture was adjusted to pH 3 with 1 M (molar concentration) HCl. Water (5 mL) was added. The mixture was extracted with ethyl acetate (5 mL). The organic phase was discarded. The aqueous phase was adjusted to pH 8 with sodium bicarbonate. The mixture was extracted with dichloromethane (5 mL×2). The combined organic phases were dried over anhydrous sodium sulfate, filtered and concentrated to give a crude product, which was separated and purified by Prep-HPLC (0.225% aqueous HCOOH, MeCN) to give the target compound (7.76 mg, 4.21%). LC-MS: [M+H]⁺=375.4; ¹H NMR (400 MHz, DMSO-d₆): δ 11.12 (brs, 1H), 8.54-8.53 (m, 1H), 8.05 (d, J=8.4 Hz, 1H), 7.24-7.22 (m, 1H), 7.17-7.15 (m, 2H), 7.07-7.06 (m, 1H), 7.02-7.00 (m, 1H), 6.71 (s, 2H), 5.45 (s, 2H), 3.48 (s, 2H), 2.30-2.27 (m, 4H), 1.62-1.54 (m, 4H).

### Example 28: Preparation of 4-amino-1-(1-(4-(pyrrolidin-1-ylmethyl)phenyl)ethyl)-1,3-dihydro-2H-imidazo[4,5-c]quinolin-2-one

### Step 1: Preparation of 1-(4-(pyrrolidin-1-ylmethyl)phenyl)ethanone

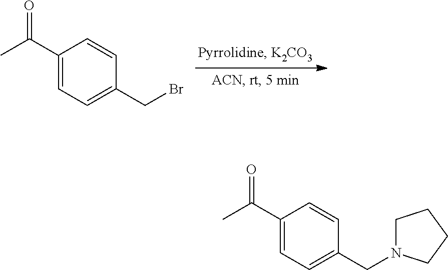

The starting material pyrrolidine (200 mg, 2.82 mmol, 3.0 eq) was dissolved in acetonitrile (5 mL). Potassium carbonate (156 mg, 1.13 mmol, 1.2 eq) was added and the mixture was stirred for 5 min, and then a solution of p-acetylbenzyl bromide (200 mg, 0.94 mmol, 1.0 eq) in acetonitrile (5 mL) was added dropwise to the reaction mixture. After the dropwise addition, the reaction mixture was stirred at 25° C. for 5 min. After the reaction was completed, as detected by TLC (DCM:MeOH=10:1), the reaction mixture was concentrated to remove the acetonitrile to give a crude product, which was purified by Prep-TLC (DCM:MeOH=10:1) to give the target compound (145 mg, 76%). LC-MS: [M+H]⁺=204.1.

### Step 2: Preparation of 1-(4-(pyrrolidin-1-ylmethyl)phenylethylamine

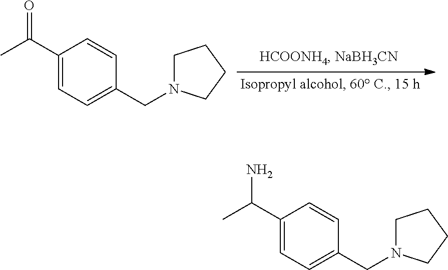

The starting material 1-(4-(pyrrolidin-1-ylmethyl)phenyl)ethanone (1.30 g, 0.64 mmol, 1 eq) was dissolved in isopropanol (15 mL). Ammonium formate (2.02 g, 31.96 mmol, 5 eq) and sodium cyanoborohydride (1.91 g, 31.96 mmol, 5 eq) were added sequentially, and then the reaction mixture was stirred at 60° C. for 15 h. After the reaction was completed, as detected by LC-MS, a small amount of water was added to the reaction mixture to quench the remaining sodium cyanoborohydride. The reaction mixture was concentrated to give a crude product, which was separated and purified by flash chromatography (DCM:MeOH=100:0 to 85:15) to give the target compound (1.1 g, 84.2%). LC-MS: [M+H]⁺=205.2.

### Step 3: Preparation of 2-chloro-3-nitro-N-(1-4-(pyrrolidin-1-ylmethyl)phenyl)ethyl)quinoline-4-amine

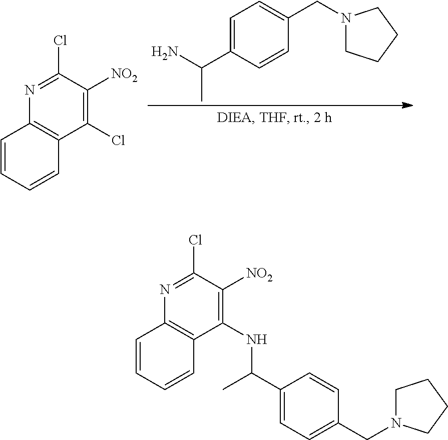 [SMILES: O=[N+]([O-])c1c(Cl)nc2ccccc2c1Cl ; CC(N)c1ccc(CN2CCCC2)cc1 ; CC(Nc1c([N+](=O)[O-])c(Cl)nc2ccccc12)c1ccc(CN2CCCC2)cc1]

The starting material 2,4-dichloro-3-nitroquinoline (1.31 g, 5.38 mol, 1 eq) was dissolved in tetrahydrofuran (5 mL). N,N-diisopropylethylamine (1.75 g, 13.56 mol, 3 eq) was added. A solution of 1-(4-(pyrrolidin-1-ylmethyl)phenylethylamine (1.10 g, 5.38 mol, 1 eq) in tetrahydrofuran (5 mL) was added dropwise to the reaction mixture under an ice bath. After the dropwise addition, the reaction mixture was warmed to room temperature and stirred at room temperature for 2 h. After the reaction was completed, as detected by LC-MS, the reaction mixture was concentrated to remove the tetrahydrofuran to give a crude product, which was separated and purified by flash chromatography (DCM:MeOH=100:0 to 90:10) to give the target compound (0.60 g, 27.1%). LC-MS: [M+H]⁺=411.1.

### Step 4: Preparation of N²,N²-bis(4-methoxybenzyl)-3-nitro-N⁴-(1-(4-(pyrrolidin-1-ylmethyl)phenyl)ethyl)quinoline-2,4-diamine

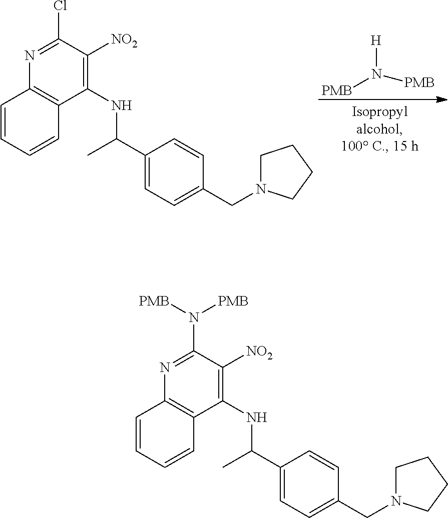

The starting material 2-chloro-3-nitro-N-(1-4-(pyrrolidin-1-ylmethyl)phenyl)ethyl)quinolin-4-amine (0.55 g, 1.34 mmol, 1 eq) was dissolved in isopropanol (8 mL), and then bis(4-methoxybenzyl)amine (0.69 g, 2.68 mmol, 2 eq) was added. The reaction mixture was refluxed at 100° C. for 15 h. After the reaction was completed, as detected by LC-MS, the reaction mixture was concentrated to remove the isopropanol to give a crude product, which was purified by slurrying with a solvent (in a ratio of PE:EA=20:1) to give the target compound (0.35 g, 41.4%). LC-MS: [M+H]⁺=632.3.

### Step 5: Preparation of N²,N²-bis(4-methoxybenzyl) N⁴-(1-(4-(pyrrolidin-1-ylmethyl)phenyl)ethyl)quinoline-2,3,4-triamine

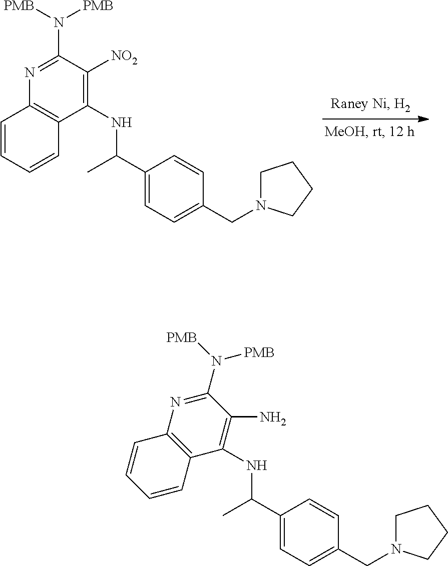

Raney nickel (139 mg, 2.37 mmol, 5.0 eq.) was added to a solution of N²,N²-bis(4-methoxybenzyl)-3-nitro-N⁴-(1-(4-(pyrrolidin-1-ylmethyl)phenyl)ethyl)quinoline-2,4-diamine (300 mg, 0.47 mmol, 1.0 eq.) in absolute methanol (10 mL) at room temperature. The mixture was stirred and reacted under hydrogen atmosphere for 12 h. After the reaction was completed, as detected by LC-MS, the mixture was filtered through celite and washed with methanol. The filtrate was concentrated to give a crude product in the form of a red oily mixture. The crude product was purified by Prep-TLC (DCM:MeOH=15:1) to give the target compound (150 mg, 53%). LC-MS: [M+H]⁺=602.3.

### Step 6: Preparation of 4-(bis(4-methoxybenzyl)amino)-1-(1-(4-(pyrrolidin-1-ylmethyl)phenyl)ethyl)-1,3-dihydro-2H-imidazo[4,5-c]quinolin-2-one

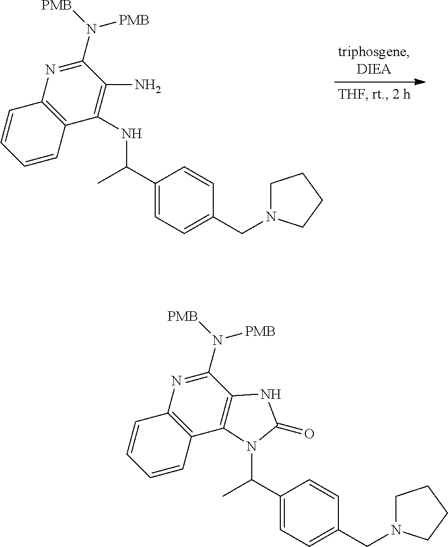

N²,N²-bis(4-methoxybenzyl)-N⁴-(1-(4-(pyrrolidin-1 ylmethyl)phenyl)ethyl)quinoline-2,3,4-triamine (120 mg, 0.20 mmol, 1.0 eq.) was dissolved in tetrahydrofuran (3 mL), and then N,N-diisopropylethylamine (77.3 mg, 0.60 mmol, 3 eq.) was added to the reaction mixture. A solution of triphosgene (23.7 mg, 0.08 mmol, 0.4 eq.) in tetrahydrofuran (1.5 mL) was added dropwise under an ice bath. After the dropwise addition, the mixture was warmed to room temperature and stirred for 2 h. After the reaction was completed, as detected by LC-MS, a saturated aqueous sodium bicarbonate solution was added to the reaction mixture to adjust the pH to 9, and then the mixture was extracted with ethyl acetate (5 mL×3). The combined organic phases were washed with saturated brine, dried over anhydrous sodium sulfate, filtered and concentrated to give a crude product (60 mg, 47.9%), which was used directly in the next step. LC-MS: [M+H]⁺=628.3.

### Step 7: Preparation of 4-amino-1-(1-(4-(pyrrolidin-1-ylmethyl)phenyl)ethyl)-1,3-dihydro-2H-imidazo[4,5-c]quinolin-2-one

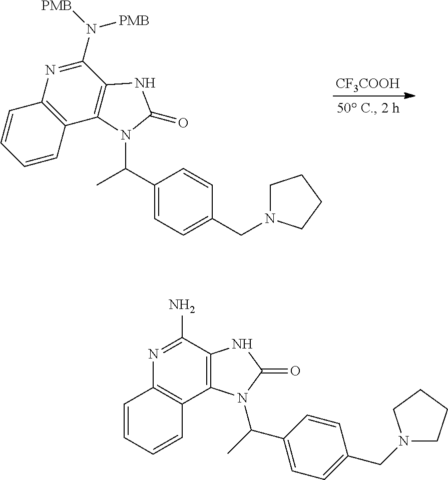

The starting material 4-(bis(4-methoxybenzyl)amino)-1-(1-(4-(pyrrolidin-1-ylmethyl)phenyl)ethyl)-1,3-dihydro-2H-imidazo[4,5-c]quinolin-2-one (60 mg, 0.10 mmol, 1.0 eq.) was dissolved in trifluoroacetic acid (3 mL). The reaction mixture was stirred at 50° C. for 1 h. After the reaction was completed, as detected by LC-MS, the reaction mixture was concentrated to remove the trifluoroacetic acid. A saturated aqueous sodium bicarbonate solution was added to adjust the pH to 9. The mixture was extracted with dichloromethane (8 mL×3). The combined organic phases were dried over anhydrous sodium sulfate, filtered and concentrated to give a crude product, which was purified by prep-HPLC (0.01% aqueous HCl solution, MeCN) to give the target compound (13 mg, 35.6%). LC-MS: [M+H]⁺=388.2; ¹H NMR (400 MHz, CD₃OD): δ 7.74-7.53 (m, 7H), 7.30 (s, 1H), 6.42 (q, J=8.0 Hz, 1H), 4.37 (s, 2H), 3.45-3.41 (m, 2H), 3.18-3.13 (m, 2H), 2.19-2.15 (m, 2H), 2.08 (d, J=8.0 Hz, 3H), 2.02-1.94 (m, 2H).

### Example 29: Preparation of 8-amino-6-butoxy-3-(3-(pyrrolidin-1-ylmethyl)benzyl)-3,4-dihydropyrimido[5,4-d]pyrimidin-2(1H)-one

### Step 1: Preparation of butyl carbamimidate

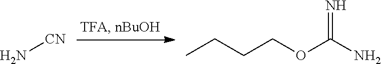

The starting material cyanamide (85.0 g, 2.02 mol, 1.0 eq) was dissolved in n-butanol (80 mL). Trifluoroacetic acid (231 g, 2.02 mol, 1.0 eq) was added at 25° C. The reaction mixture was stirred at 25° C. for 12 h. After the reaction was completed, as detected by TLC (DCM:MeOH=10:0), the reaction mixture was concentrated to give the target compound (145 g, 61.7%). ¹H NMR (400 MHz, DMSO-d₆): δ 4.22 (t, J=6.6 Hz, 2H), 1.69-1.62 (m, 2H), 1.40-1.32 (m, 2H), 0.91 (t, J=7.4 Hz, 3H).

### Step 2: Preparation of 2-butoxypyrimidin-4,6-diol

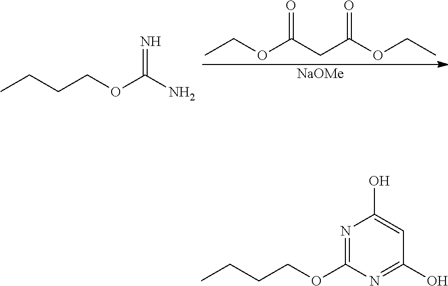

The starting material butyl carbamimidate (125 g, 819 mmol, 1.0 eq) was dissolved in methanol (1 L). Sodium methoxide (5.4 M, 455 mL, 3.0 eq) was added at −5° C. Diethyl malonate (108 g, 821 mmol, 1.0 eq) was added at 0° C. The mixture was stirred at 25° C. for 12 h. After the reaction was completed, as detected by TLC (DCM:MeOH=10:1), the reaction mixture was adjusted to pH 4 with 1 M (molar concentration) hydrochloric acid and filtered to give a solid. The solid was dried to give the target compound (45.0 g, 29.8%). ¹H NMR (400 MHz, DMSO-d₆): δ 11.52 (brs, 2H), 4.96 (s, 1H), 4.24 (t, J=6.6 Hz, 2H), 1.65-1.59 (m, 2H), 1.38-1.33 (m, 2H), 0.90 (t, J=7.2 Hz, 3H).

### Step 3: Preparation of 2-butoxy-5-nitropyrimidin-4,6-diol

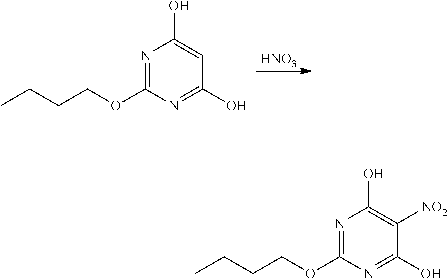

The starting material 2-butoxypyrimidin-4,6-diol (40.0 g, 217 mmol, 1.0 eq) was dissolved in acetic acid (300 mL), and then fuming nitric acid (210 mL) was added at −5° C. The reaction mixture was stirred at 25° C. for 1 h. After the reaction was completed, as detected by LC-MS, the reaction mixture was added dropwise to ice water (800 mL). The mixture was extracted with ethyl acetate (500 mL×3). The combined organic phases were washed with saturated brine (500 mL), dried over anhydrous sodium sulfate, filtered and concentrated to give the target compound (35.5 g, 71.3%). LC-MS: [M+H]⁺=203.08.

### Step 4: Preparation of 2-butoxy-4,6-dichloro-5-nitropyrimidine

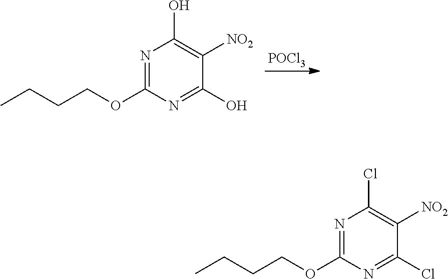

The starting material 2-butoxy-5-nitropyrimidin-4,6-diol (35.5 g, 155 mmol, 1.0 eq) was dissolved in phosphorus oxychloride (142 mL). The reaction mixture was warmed to 40° C. N,N-diethylaniline (57.8 g, 387 mmol, 2.5 eq) was added. The mixture was stirred at 60° C. for 3 h. After the reaction was completed, as detected by TLC (PE:EA=5:1), the reaction mixture was cooled to room temperature and slowly added to warm water (800 mL). The mixture was extracted with dichloromethane (500 mL×3). The combined organic phases were washed with saturated brine (500 mL), dried over anhydrous sodium sulfate, filtered and concentrated to give a crude product, which was separated and purified by flash chromatography (PE:EA=1:0 to 3:1) to give the target compound (37.8 g, 91.7%). LC-MS: [M+H]⁺=266.08.

### Step 5: Preparation of 2-butoxy-6-chloro-N,N-bis(4-methoxybenzyl)-5-nitropyrimidin-4-amine

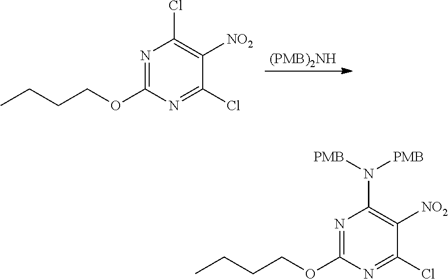

The starting material 2-butoxy-4,6-dichloro-5-nitropyrimidine (37.8 g, 142 mmol, 1.0 eq) was dissolved in anhydrous tetrahydrofuran (300 mL), and then triethylamine (21.6 g, 213 mmol, 1.5 eq) and 1-(4-methoxyphenyl)-N-[(4-methoxyphenyl)methyl]methylamine (36.6 g, 142 mmol, 1.0 eq) were added. The reaction mixture was stirred at 25° C. for 4 h. After the reaction was completed, as detected by TLC (PE:EtOAc=5:1), the reaction mixture was filtered, and water (800 mL) was added to the filtrate. The mixture was extracted with ethyl acetate (500 mL×3). The combined organic phases were washed with saturated brine (500 mL), dried over anhydrous sodium sulfate, filtered and concentrated to give the product. The product was separated and purified by flash chromatography (PE:EtOAc=1:0 to 3:1) to give the target compound (66.8 g, 96.6%). LC-MS: [M+H]⁺=487.30.

### Step 6: Preparation of 2-butoxy-6-chloro-N⁴,N⁴-bis(4-methoxybenzyl)pyrimidin-4,5-diamine

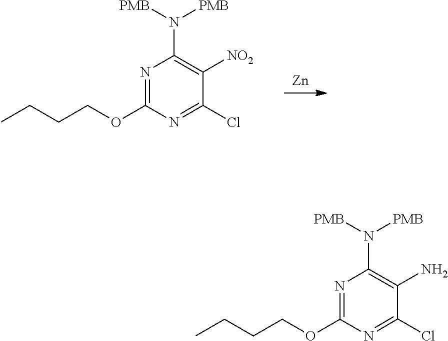

The starting materials 2-butoxy-6-chloro-N,N-bis(4-methoxybenzyl)-5-nitropyrimidin-4-amine (10.0 g, 20.5 mmol, 1.0 eq) and zinc powder (6.71 g, 103 mmol, 5.0 eq) were dissolved in methanol (60 mL), water (30 mL), and tetrahydrofuran (60 mL). Ammonium chloride (5.49 g, 103 mmol, 5.0 eq) was added at 25° C. The reaction mixture was stirred at 25° C. for 4 h. After the reaction was completed, as detected by TLC (PE:EtOAc=5:1), the reaction mixture was filtered, and the filter cake was washed with dichloromethane. The mixture was extracted with dichloromethane (150 mL×3). The combined organic phases were washed with saturated brine (100 mL), dried over anhydrous sodium sulfate, filtered and concentrated to give a crude product, which was separated and purified by flash chromatography (PE:EtOAc=1:0 to 3:1) to give the target compound (3.70 g, 39.4%). LC-MS: [M+H]⁺=457.16.

### Step 7: Preparation of methyl 5-amino-6-(bis(4-methoxybenzyl)amino)-2-butoxypyrimidin-4-carboxylate

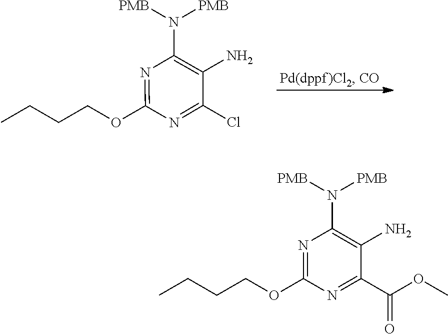

The starting materials 2-butoxy-6-chloro-N⁴,N⁴-bis(4-methoxybenzyl)pyrimidin-4,5-diamine (3.70 g, 8.10 mmol, 1.0 eq), and triethylamine (2.46 g, 24.3 mmol, 3.0 eq) were dissolved in methanol (60 mL). [1,1-bis(diphenylphosphino)ferrocene]dichloropalladium(II) (592 mg, 809 mmol, 0.1 eq) was added. The reaction mixture was purged with nitrogen 3 times, purged with carbon monoxide 3 times and stirred at 80° C. for 16 h under carbon monoxide atmosphere (40 Psi). After the reaction was completed, as detected by LC-MS, the reaction mixture was filtered. The filter cake was washed with methanol. The filtrate was concentrated to give a crude product, which was separated and purified by flash chromatography (PE:EtOAc=1:0 to 3:1) to give the target compound (3.60 g, 92.5%). LC-MS: [M+H]⁺=481.24.

### Step 8: Preparation of (5-amino-6-(bis(4-methoxybenzyl)amino)-2-butoxypyrimidin-4-yl)methanol

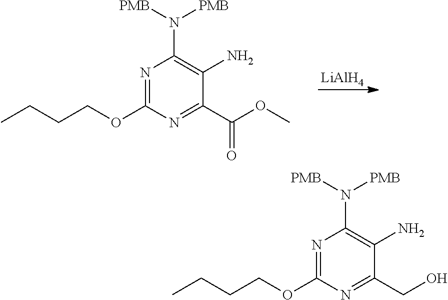

The starting materials methyl 5-amino-6-(bis(4-methoxybenzyl)amino)-2-butoxypyrimidin-4-carboxylate (2.30 g, 4.79 mmol, 1.0 eq) and methanol (307 mg, 9.57 mmol, 2.0 eq) were dissolved in tetrahydrofuran (20 mL). Lithium borohydride (209 mg, 9.57 mmol, 2.0 eq) was added at 25° C. The reaction mixture was stirred at 70° C. for 2 h. After the reaction was completed, as detected by LC-MS, the reaction was cooled to room temperature and quenched with 1 M hydrochloric acid (30 mL) at 0° C. The mixture was extracted with ethyl acetate (50 mL×3). The combined organic phases were washed with saturated brine (50 mL), dried over anhydrous sodium sulfate, filtered and concentrated to give the target compound (2.00 g, 92.3%). LC-MS: [M+H]⁺=453.1.

### Step 9: Preparation of tert-butyl (4-(bis(4-methoxybenzyl)amino)-2-butoxy-6-(hydroxymethyl)pyrimidin-5-yl)carbamate

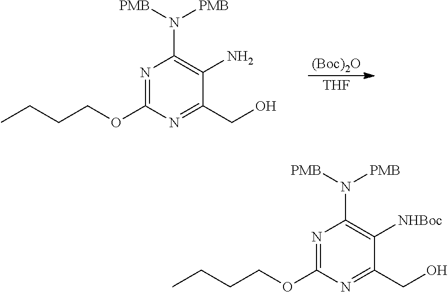

The starting materials (5-amino-6-(bis(4-methoxybenzyl)amino)-2-butoxypyrimidin-4-yl)methanol (1.50 g, 3.31 mmol, 1.0 eq) and di-tert-butyl dicarbonate (1.81 g, 8.29 mmol, 2.5 eq) were dissolved in tetrahydrofuran (20 mL). The reaction mixture was stirred at 60° C. for 24 h. After the reaction was completed, as detected by LC-MS, the reaction was concentrated to give the target compound (1.50 g, 81.89%). LC-MS: [M+H]⁺=553.4.

### Step 10: Preparation of tert-butyl (4-(bis(4-methoxybenzyl)amino)-6-(bromomethyl)-2-butoxypyrimidin-5-yl)carbamate

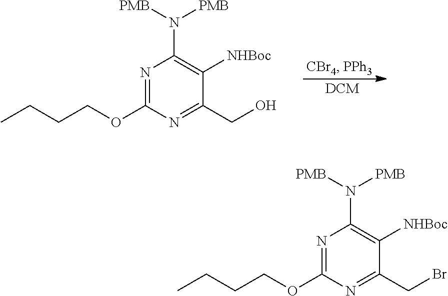

The starting materials tert-butyl (4-(bis(4-methoxybenzyl)amino)-2-butoxy-6-(hydroxymethyl)pyrimidin-5-yl)carbamate (500 mg, 905 μmol, 1.0 eq) and tetrabromomethane (510 mg, 1.54 mmol, 1.7 eq) were dissolved in dichloromethane (10 mL). Triphenylphosphine (403 mg, 1.54 mmol, 1.7 eq) was added at 25° C. The reaction mixture was stirred at 25° C. for 2 h. After the reaction was completed, as detected by LC-MS, the reaction mixture was added to water (10 mL). The mixture was extracted with dichloromethane (10 mL×3). The combined organic phases were washed with saturated brine (20 mL), dried over anhydrous sodium sulfate, filtered and concentrated to give the product. The product was separated and purified by flash chromatography (DCM:MeOH=1:0 to 10:1) to give the target compound (480 mg, 86.2%). LC-MS: [M+H]⁺=617.41.

### Step 11: Preparation of tert-butyl (4-(bi s(4-methoxybenzyl)amino)-2-butoxy-6-(((3-(pyrrolidin-1-ylmethyl)benzyl)amino)methyl)pyrimidin-5-yl)carbamate

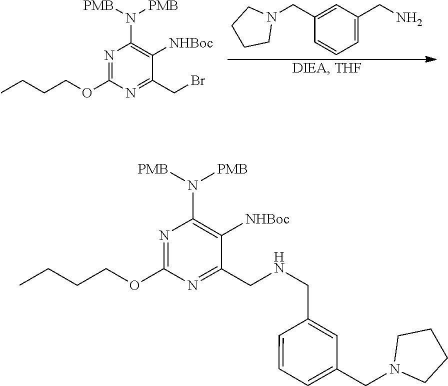

The starting materials tert-butyl (4-(bis(4-methoxybenzyl)amino)-6-(bromomethyl)-2-butoxypyrimidin-5-yl)carbamate (180 mg, 292 μmol, 1.0 eq) and [3-(pyrrolidin-1-ylmethyl)phenyl]methylamine (55.6 mg, 292 μmol, 1.0 eq) were dissolved in tetrahydrofuran (10 mL). N,N-diisopropylethylamine (113 mg, 877 μmol, 3.0 eq) was added at 25° C. The reaction mixture was stirred at 25° C. for 12 h. After the reaction was completed, as detected, the reaction mixture was added to water (10 mL). The mixture was extracted with dichloromethane (10 mL×3). The combined organic phases were washed with saturated brine (20 mL), dried over anhydrous sodium sulfate, filtered and concentrated to give the product. The product was separated and purified by flash chromatography (DCM:MeOH=1:0 to 10:1) to give the target compound (120 mg, 56.6%). LC-MS: [M+H]⁺=725.21.

### Step 12: Preparation of 8-(bis(4-methoxybenzyl)amino)-6-butoxy-3-(3-(pyrrolidin-1-ylmethyl)benzyl)-3,4-dihydropyrimido[5,4-d]pyrimidin-2(1H)-one

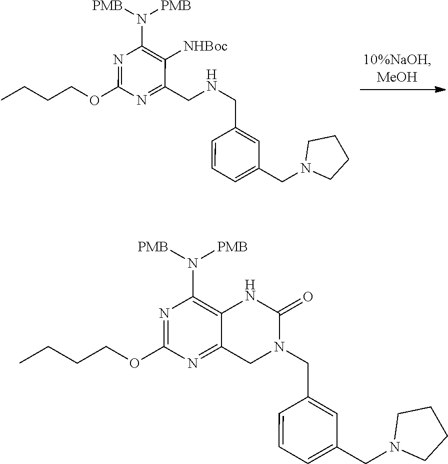

The starting material tert-butyl (4-(bis(4-methoxybenzyl)amino)-2-butoxy-6-(((3-(pyrrolidin-1-ylmethyl)benzyl)amino)methyl)pyrimidin-5-yl)carbamate (120 mg, 166 μmol, 1.0 eq) was dissolved in methanol (5 mL) and sodium hydroxide (1 mL, 10%). The reaction mixture was stirred at 70° C. for 6 h. After the reaction was completed, as detected by LC-MS, the reaction mixture was added to water (5 mL). The mixture was extracted with ethyl acetate (10 mL×3). The combined organic phases were washed with saturated brine (20 mL), dried over anhydrous sodium sulfate, filtered and concentrated to give a crude product. The target compound (100 mg, 56.6%) was obtained by prep-TLC (DCM:MeOH=20:1). LC-MS: [M+H]⁺=651.4.

### Step 13: Preparation of 6-butoxy-8-[(4-methoxybenzyl)amino]-3-[3-(pyrrolidin-1-ylmethyl)benzyl]-1,4-dihydropyrimido[5,4-d]pyrimidin-2-one

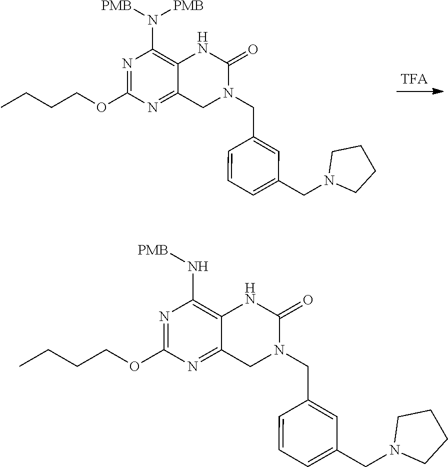

The starting material 8-(bis(4-methoxybenzyl)amino)-6-butoxy-3-(3-(pyrrolidin-1-ylmethyl)benzyl)-3,4-dihydropyrimido[5,4-d]pyrimidin-2(1H)-one (100 mg, 154 μcool, 1.0 eq) was dissolved in trifluoroacetic acid (2 mL). The reaction mixture was stirred at 50° C. for 2 h. After the reaction was completed, as detected by LC-MS, the reaction mixture was concentrated to give the target compound (60 mg, 73.6%). LC-MS: [M+H]⁺=531.11.

### Step 14: Preparation of 8-amino-6-butoxy-3-(3-(pyrrolidin-1-ylmethyl)benzyl)-3,4-dihydropyrimido[5,4-d]pyrimidin-2 (1H)-one

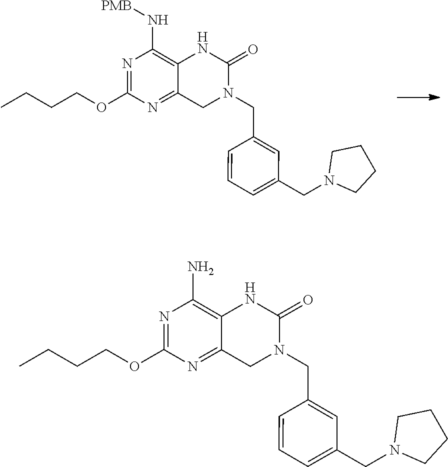

The starting material 6-butoxy-8-[(4-m ethoxybenzyl)amino]-3-[3-(pyrrolidin-1-ylmethyl)benzyl]-1,4-dihydropyrimido[5,4-d]pyrimidin-2-one (30.0 mg, 56.5 μcool, 1.0 eq) was dissolved in trifluoroacetic acid (2 mL). Trifluoromethanesulfonic acid (0.2 mL) was added at 25° C. The reaction mixture was stirred at 50° C. for 2 h. After the reaction was completed, as detected by LC-MS, the reaction mixture was concentrated to give a crude product, which was separated and purified by Prep-HPLC (0.225% aqueous HCOOH, ACN) to give the target compound (2.76 mg, 10.8%). LC-MS: [M+H]⁺=411.41; ¹H NMR (400 MHz, MeOH-d₄): δ 8.51 (s, 1H), 7.51-7.49 (m, 3H), 7.46-7.45 (m, 1H), 4.68 (s, 2H), 4.31 (s, 2H), 4.25 (s, 2H), 4.21 (t, J=6.4 Hz, 2H), 3.25 (brs, 4H), 2.07 (brs, 4H), 1.72-1.68 (m, 2H), 1.49-1.43 (m, 2H), 0.97 (t, J=7.4 Hz, 3H).

### Example 30: Preparation of 8-amino-6-butoxy-3-(2-(pyrrolidin-1-ylmethyl)benzyl)-3,4-dihydropyrimido[5,4-d]pyrimidin-2(1H)-one

### Step 1: Preparation of tert-butyl (4-(bis(4-methoxybenzyl)amino)-2-butoxy-6-(((2-(pyrrolidin-1-ylmethyl)benzyl)amino)methyl)pyrimidin-5-yl)carbamate

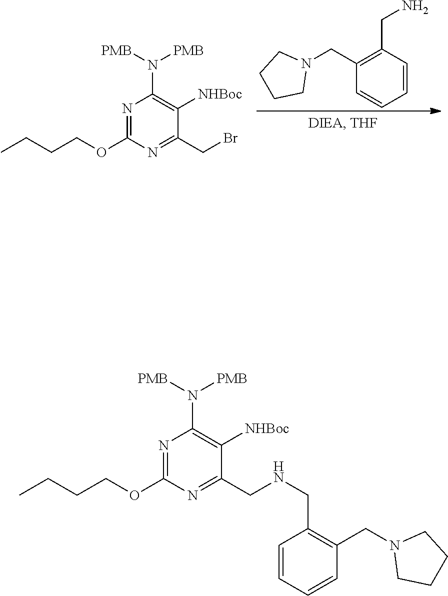

The starting materials tert-butyl (4-(bis(4-methoxybenzyl)amino)-6-(bromomethyl)-2-butoxypyrimidin-5-yl)carbamate (400 mg, 649 μmol, 1.0 eq) and (2-(pyrrolidin-1-ylmethyl)phenyl)methylamine (124 mg, 650 μmol, 1.0 eq) were dissolved in tetrahydrofuran (5 mL). N,N-diisopropylethylamine (252 mg, 1.95 mmol, 3.0 eq) was added at 25° C. The reaction mixture was stirred at 25° C. for 12 h. After the reaction was completed, as detected by LC-MS, the reaction mixture was added to water (10 mL). The mixture was extracted with dichloromethane (10 mL×3). The combined organic phases were washed with saturated brine (20 mL), dried over anhydrous sodium sulfate, filtered and concentrated to give a crude product, which was separated and purified by flash chromatography (DCM:MeOH=1:0 to 10:1) to give the target compound (200 mg, 42.5%). LC-MS: [M+H]⁺=725.41.

### Step 2: Preparation of 8-(bis(4-methoxybenzyl)amino)-6-butoxy-3-(2-(pyrrolidin-1-ylmethyl)benzyl)-3,4-dihydropyrimido[5,4-d]pyrimidin-2(1H)-one

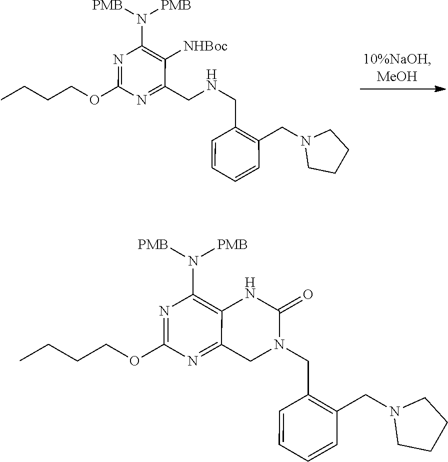

The starting material tert-butyl (4-(bis(4-methoxybenzyl)amino)-2-butoxy-6-(((2-(pyrrolidin-1-ylmethyl)benzyl)amino)methyl)pyrimidin-5-yl)carbamate (180 mg, 248 μmol, 1.0 eq) was dissolved in methanol (5 mL) and sodium hydroxide (1 mL, 10%). The reaction mixture was stirred at 95° C. for 12 h. After the reaction was completed, as detected by LC-MS, the reaction mixture was concentrated to give the target compound (148 mg, 91.59%). LC-MS: [M+H]⁺=651.21.

### Step 3: Preparation of 6-butoxy-8-((4-methoxybenzyl)amino)-3-(2-(pyrrolidin-1-ylmethyl)benzyl)-3,4-dihydropyrimido[5,4-d]pyrimidin-2(1H)-one

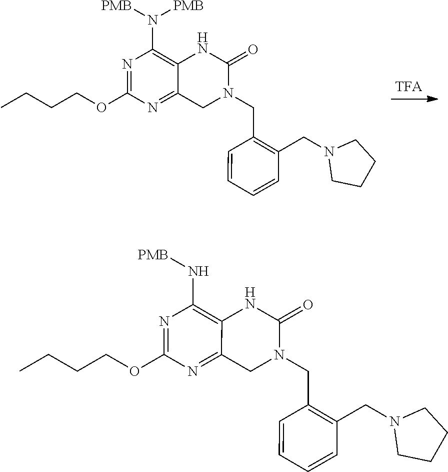

The starting material 8-(bis(4-methoxybenzyl)amino)-6-butoxy-3-(2-(pyrrolidin-1-ylmethyl)benzyl)-3,4-dihydropyrimido[5,4-d]pyrimidin-2(1H)-one (145 mg, 223 μmol, 1.0 eq) was dissolved in trifluoroacetic acid (3 mL). The reaction mixture was stirred at 50° C. for 2 h. After the reaction was completed, as detected by LC-MS, the reaction mixture was concentrated to give the target compound (120 mg, crude product). LC-MS: [M+H]⁺=531.31.

### Step 4: Preparation of 8-amino-6-butoxy-3-(2-(pyrrolidin-1-ylmethyl)benzyl)-3,4-dihydropyrimido[5,4-d]pyrimidin-2 (1H)-one

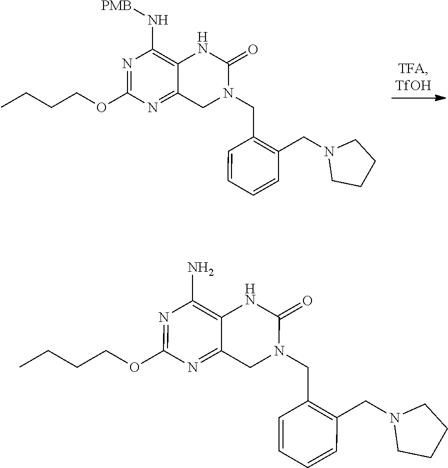

The starting material 6-butoxy-8-((4-methoxybenzyl)amino)-3-(2-(pyrrolidin-1-ylmethyl)benzyl)-3,4-dihydropyrimido[5,4-d]pyrimidin-2(1H)-one (300 mg, 565 μmol, 1.0 eq) was dissolved in trifluoroacetic acid (3 mL). Trifluoromethanesulfonic acid (0.2 mL) was added at 25° C. The reaction mixture was stirred at 50° C. for 2 h. After the reaction was completed, as detected by LC-MS, the reaction mixture was concentrated to give a crude product, which was separated and purified by Prep-HPLC (0.05% aqueous HCl solution, ACN) to give the target compound (23.02 mg, 9.39%). LC-MS: [M+H]⁺=411.41; ¹H NMR (400 MHz, MeOH-d₄): δ 7.55-7.51 (m, 3H), 7.49-7.47 (m, 1H), 4.76 (s, 2H), 4.55 (s, 2H), 4.50 (s, 2H), 4.38-4.33 (m, 2H), 3.56-3.55 (brs, 2H), 3.24-3.21 (m, 2H), 2.24-2.21 (m, 2H), 2.02 (brs, 2H), 1.76-1.72 (brs, 2H), 1.49-1.44 (m, 2H), 0.97 (t, J=7.4 Hz, 3H).

### Example 31: Preparation of 4-amino-1-(3-methyl-4-(pyrrolidin-1-ylmethyl)benzyl)-1,3-dihydro-2H-imidazo[4,5-c]quinolin-2-one

### Step 1: Preparation of 4-(hydroxymethyl)-3-methylbenzonitrile

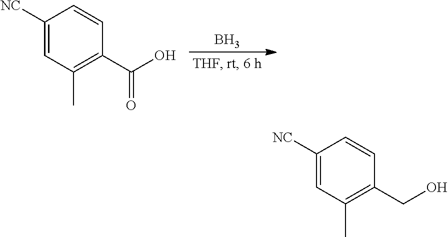

The starting material 4-cyano-2-methylbenzoic acid (3.00 g, 18.62 mmol, 1 eq.) was added to a 250-mL three-necked flask. Extra dry THF (30 mL) was injected into the three-necked flask using a syringe. Borane dimethyl sulfide coordination complex (37.24 mL, 74.48 mmol, 2 M tetrahydrofuran solution, 4 eq.) was added dropwise to the reaction mixture under an ice bath. After the dropwise addition, the mixture was slowly warmed to room temperature and successively stirred for 4 h. After the reaction was completed, as detected by LC-MS, absolute methanol was slowly added dropwise to the reaction mixture under an ice bath until no bubbles were generated. The reaction mixture was concentrated to give a crude product, which was separated and purified by flash chromatography (DCM:MeOH=100:0 to 94:7) to give the target compound (1.2 g, 43.8%). LC-MS: [M+H]⁺=148.10.

### Step 2: Preparation of 4-(chloromethyl)-3-methylbenzonitrile

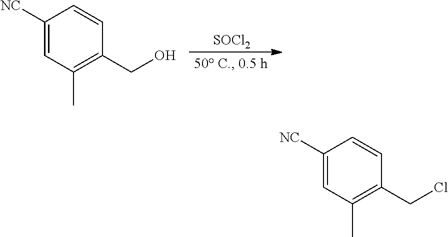

The starting material 4-(hydroxymethyl)-3-methylbenzonitrile (1.00 g, 6.80 mmol, 1 eq.) was dissolved in thionyl chloride (10 mL). The reaction mixture was heated and stirred at 50° C. for half an hour. After the reaction was completed, as detected by TLC, the reaction mixture was concentrated under reduced pressure to remove excessive thionyl chloride to give a crude product (1 g, 88.9%), which was used directly in the next step.

### Step 3: Preparation of 3-methyl-4-(pyrrolidin-1-ylmethyl)benzonitrile

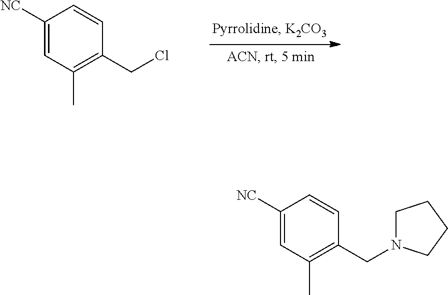

Pyrrolidine (1.29 g, 18.12 mmol, 3 eq.) was dissolved in acetonitrile (5 mL). Potassium carbonate (1.00 g, 7.25 mol, 1.2 eq.) was added. The mixture was stirred for 5 min. 4-(chloromethyl)-3-methylbenzonitrile (1.00 g, 6.04 mol, 1 eq.) was dissolved in acetonitrile (5 mL) and added dropwise to the reaction mixture described above. After the dropwise addition, the reaction mixture was stirred at 25° C. for 5 min. After the reaction was completed, as detected by TLC, the reaction mixture was concentrated to remove the acetonitrile to give a crude product, which was separated and purified by flash chromatography (DCM:MeOH=100:0 to 94:6) to give the target compound (0.91 g, 75.3%). LC-MS: [M+H]⁺=201.10.

### Step 4: Preparation of (3-methyl-4-(pyrrolidin-1-ylmethyl)phenylmethylamine

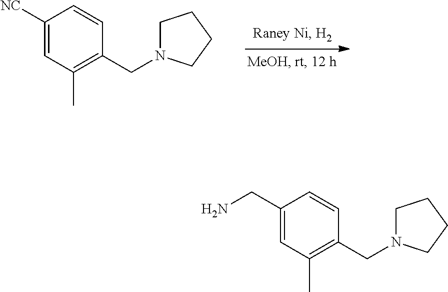

The starting material 3-methyl-4-(pyrrolidin-1-ylmethyl)benzonitrile (700 mg, 3.50 mmol, 1 eq.) was dissolved in a solution of methylamine in ethanol (5 mL), and then Raney nickel (1.02 g, 17.5 mmol, 5 eq.) was added to the mixture described above. The mixture was stirred and reacted under hydrogen atmosphere for 12 h. After the reaction was completed, as detected by LC-MS, the mixture was filtered through celite and the filter cake was washed with methanol. The filtrate was concentrated to give a crude product (0.75 g, 92%), which was used directly in the next step. LC-MS: [M+H]⁺=205.10.

### Step 5: Preparation of 2-chloro-N-(3-methyl-4-(pyrrolidin-1-ylmethyl)benzyl)-3-nitroquinoline-4-amine

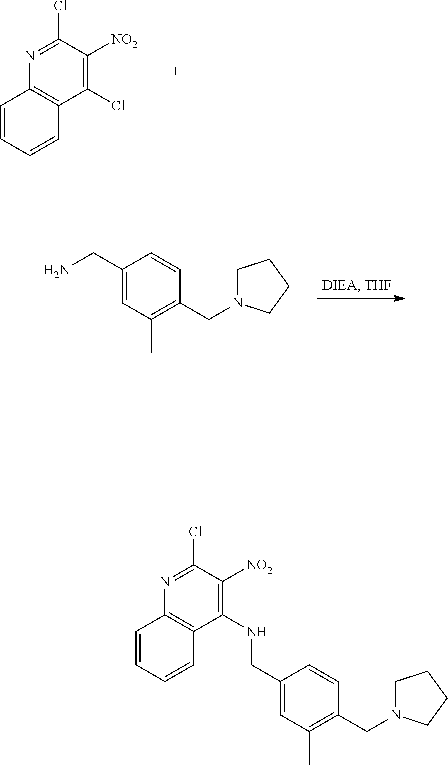 [SMILES: O=[N+]([O-])c1c(Cl)nc2ccccc2c1Cl ; Cc1cc(CN)ccc1CN1CCCC1 ; Cc1cc(CNc2c([N+](=O)[O-])c(Cl)nc3ccccc23)ccc1CN1CCCC1]

The starting material 2,4-dichloro-3-nitroquinoline (833 mg, 3.43 mmol, 1 eq) was dissolved in tetrahydrofuran (5 mL), and then N,N-diisopropylethylamine (1.33 g, 10.29 mmol, 3 eq) was added. (3-methyl-4-(pyrrolidin-1-ylmethyl)phenylmethylamine (700 mg, 3.43 mmol, 1 eq.) was dissolved in tetrahydrofuran (5 mL) and was added dropwise to the reaction mixture described above under an ice bath. After the dropwise addition, the reaction mixture was transferred to room temperature and was stirred at room temperature for 2 h. After the reaction was completed, as detected by LC-MS, the reaction mixture was concentrated to remove the tetrahydrofuran, and then water (10 mL) was added to the reaction mixture. The mixture was extracted with dichloromethane (20 mL×3). The organic phases were combined, dried over anhydrous sodium sulfate, filtered and concentrated to give a crude product, which was purified using a C18 column (0.01% aqueous formic acid:CAN=100:0 to 73:27) to give the target compound (400 mg, 28.4%). LC-MS: [M+H]+=411.20.

### Step 6: Preparation of 12,12-bis(4-methoxybenzyl)-N⁴-(3-methyl-4-(pyrrolidin-1-ylmethyl)benzyl)-3-nitroquinoline-2,4-diamine

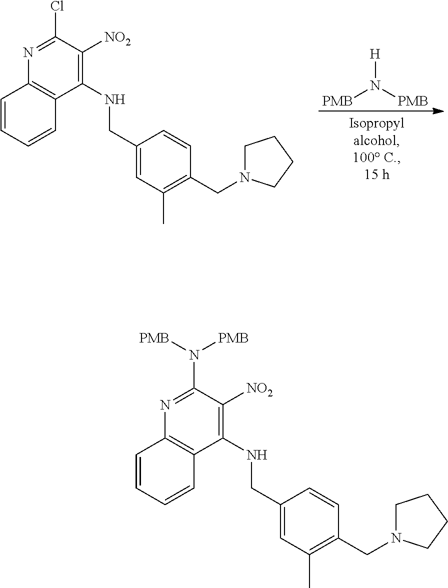

The starting material 2-chloro-N-(3-methyl-4-(pyrrolidin-1-ylmethyl)benzyl)-3-nitroquinolin-4-amine (360 mg, 0.88 mmol, 1 eq.) was dissolved in isopropanol (6 mL). Bis(4-methoxybenzyl)amine (450 mg, 1.76 mmol, 2 eq.) was added. The reaction mixture was heated and stirred at 110° C. for 50 h. After the reaction was completed, as detected by LC-MS, the reaction mixture was concentrated to remove the isopropanol to give a crude product, which was purified using a C18 column (0.01% aqueous formic acid:ACN=100:0 to 74:26) to give the target compound (400 mg, 72.3%). LC-MS: [M+H]⁺=632.5.

### Step 7: Preparation of N²,N²-bis(4-methoxybenzyl)-N⁴-(3-methyl-4-(pyrrolidin-1-ylmethyl)benzyl)quinoline-2,3,4-triamine

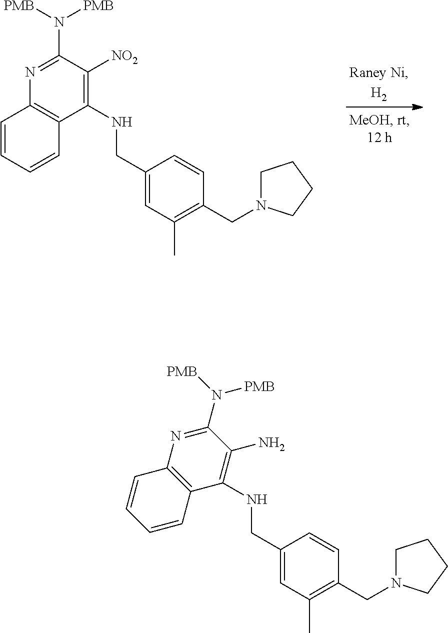

Raney nickel (334 mg, 5.70 mmol, 10 eq.) was added to a solution of N²,N²-bis(4-methoxybenzyl)-N⁴-(3-methyl-4-(pyrrolidin-1-ylmethyl)benzyl)-3-nitroquinoline-2,4-diamine (360 mg, 0.57 mmol, 1 eq.) in absolute methanol (5 mL) at room temperature. The mixture was stirred and reacted under hydrogen atmosphere for 12 h. After the reaction was completed, as detected by LC-MS, the mixture was filtered through celite and the filter cake was washed with methanol. The filtrate was concentrated to give a crude product (220 mg, 64.2%), which was used directly in the next step. LC-MS: [M+H]⁺=602.21.

### Step 8: Preparation of 4-(bis(4-methoxybenzyl)amino)-1-(3-methyl-4-(pyrrolidin-1-ylmethyl)benzyl)1,3-dihydro-2H-imidazo[4,5-c]quinolin-2-one

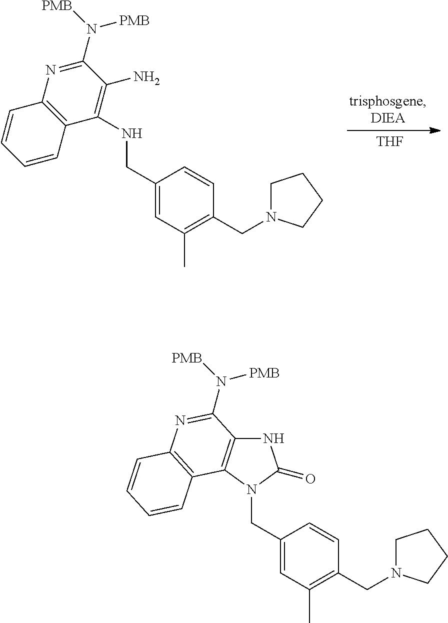

N²,N²-bis(4-methoxybenzyl)-N⁴-(3-methyl-4-(pyrrolidin-1 ylmethyl)benzyl)quinoline-2,3,4-triamine (220 mg, 0.37 mmol, 1 eq.) was dissolved in tetrahydrofuran (2 mL), and then N,N-diisopropylethylamine (142 mg, 1.11 mmol, 3 eq.) was added. Triphosgene (43 mg, 0.15 mmol, 0.4 eq.) dissolved in tetrahydrofuran (1 mL) was added dropwise under an ice bath. After the dropwise addition, the mixture was warmed to room temperature and successively stirred for 2 h. After the reaction was completed, as detected by LC-MS, a saturated aqueous sodium bicarbonate solution was added to the reaction mixture to adjust the pH to 9, and then the mixture was extracted with ethyl acetate (8 mL×3). The combined organic phases were dried over anhydrous sodium sulfate, filtered and concentrated to give a crude product (200 mg, 87.1%), which was used directly in the next step. LC-MS: [M+H]⁺=628.30.

### Step 9: Preparation of 4-amino-1-(3-methyl-4-(pyrrolidin-1-ylmethyl)benzyl)-1,3-dihydro-2H-imidazo[4,5-c]quinolin-2-one

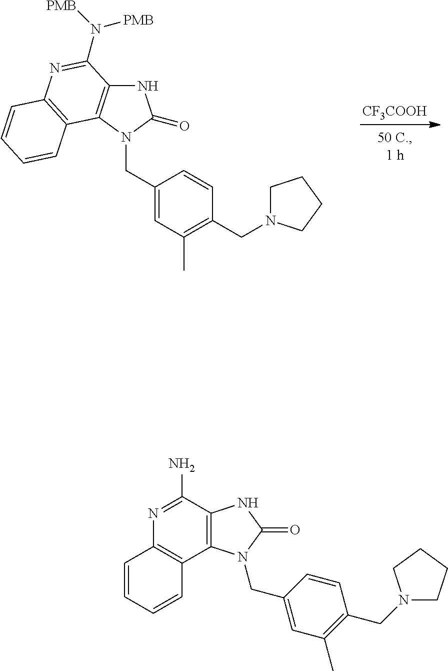

The starting material 4-(bis(4-methoxybenzyl)amino)-1-(3-methyl-4-(pyrrolidin-1-ylmethyl)benzyl)-1,3-dihydro-2H-imidazo[4,5-c]quinolin-2-one (200 mg, 0.32 mmol, 1 eq.) was dissolved in trifluoroacetic acid (2 mL). The reaction mixture was stirred at 50° C. for 1 h. After the reaction was completed, as detected by LC-MS, the reaction mixture was concentrated to remove the trifluoroacetic acid. A saturated aqueous sodium bicarbonate solution was added to the reaction mixture to adjust the pH to 9. The mixture was extracted with dichloromethane (10 mL×3), and the organic phases were combined, dried over anhydrous sodium sulfate, filtered and concentrated to give a crude product, which was separated and purified by Prep-HPLC (0.05% aqueous HCl solution, ACN) to give the target compound (10.45 mg, 8.5%). LC-MS: [M+H]⁺=388.10; ¹H NMR (400 MHz, DMSO-d₆): δ 14.09 (s, 1H), 12.39 (s, 1H), 10.43 (s, 1H), 8.65 (s, 2H), 7.89 (d, J=8.0 Hz, 1H), 7.78 (d, J=8.0 Hz, 1H), 7.63 (t, J=8.0 Hz, 1H), 7.56 (d, J=8.0 Hz, 1H), 7.37 (t, J=8.0 Hz, 1H), 7.20 (s, 1H), 7.16 (d, J=8.0 Hz, 1H), 5.52 (s, 2H), 4.31 (d, J=5.6 Hz, 2H), 3.35 (s, 2H), 3.10-3.02 (m, 2H), 2.37 (s, 3H), 2.04-1.96 (m, 2H), 1.91-1.83 (m, 2H).

### Example 32: Preparation of 4-amino-1-(3-chloro-4-(pyrrolidin-1-ylmethyl)benzyl)-1,3-dihydro-2H-imidazo[4,5-c]quinolin-2-one

### Step 1: Preparation of 1-(4-bromo-2-chlorobenzyl)pyrrolidine

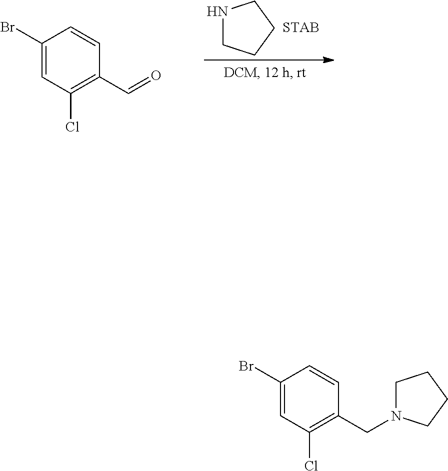 [SMILES: O=Cc1ccc(Br)cc1Cl ; C1CCNC1 ; Clc1cc(Br)ccc1CN1CCCC1]

The compound 4-bromo-2-chlorobenzaldehyde (5 g, 22.78 mmol, 1 eq) was dissolved in dichloromethane (50 mL). Tetrahydropyrrole (1.8 g, 22.06 mmol, 1.1 eq) was added, and then sodium triacetoxyborohydride (9.6 g, 45.56 mmol, 2 eq) was added. The reaction mixture was stirred at 25° C. for 12 h. After the reaction was completed, as detected by LC-MS, H₂O (50 mL) was added to the reaction system. The mixture was extracted with dichloromethane (30 mL×3), and the organic phases were combined, dried over anhydrous sodium sulfate, filtered and concentrated to give the target compound (6.3 g, 100%). The crude product was used directly in the next step. LC-MS: [M+H]⁺=276.10.

### Step 2: Preparation of 3-chloro-4-(pyrrolidin-1-ylmethyl)benzonitrile

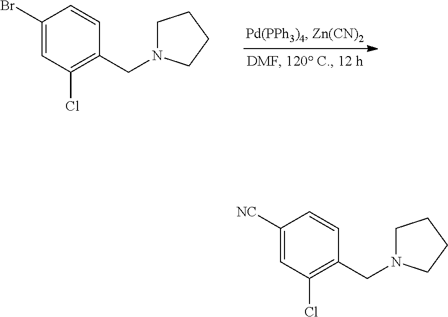 [SMILES: Clc1cc(Br)ccc1CN1CCCC1 ; N#Cc1ccc(CN2CCCC2)c(Cl)c1]

The compound 1-(4-bromo-2-chlorobenzyl)pyrrolidine (5 g, 18.21 mmol, 1 eq) was dissolved in N,N-dimethylformamide (50 mL). Zinc cyanide (4.3 g, 36.42 mmol, 2 eq) and tetrakis(triphenylphosphine)palladium(0) (2.1 g, 1.82 mmol, 0.1 eq) were added. The reaction mixture was stirred at 120° C. for 5 h under nitrogen atmosphere. After the reaction was completed, as detected by LC-MS, the reaction mixture was cooled to room temperature and concentrated. Water (100 mL) was added, and then the mixture was extracted with dichloromethane (200 mL×3). The organic phases were combined, dried over anhydrous sodium sulfate, filtered and concentrated. The crude product was separated and purified by flash chromatography (DCM:MeOH=1:0 to 10:1) to give the product (2.8 g, 69.7%). LC-MS: [M+H]⁺=221.10.

### Step 3: Preparation of (3-chloro-4-(pyrrolidin-1-ylmethyl)phenyl)methylamine

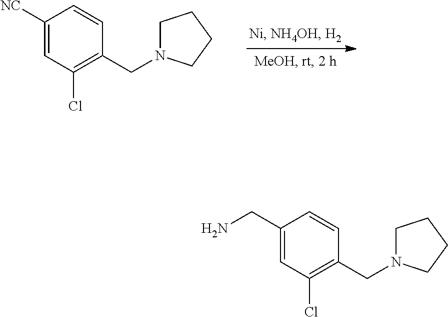 [SMILES: N#Cc1ccc(CN2CCCC2)c(Cl)c1 ; NCc1ccc(CN2CCCC2)c(Cl)c1]

The compound 3-chloro-4-(pyrrolidin-1-ylmethyl)benzonitrile (2 g, 9.06 mmol, 1 eq) was dissolved in methanol (10 mL), and then aqueous ammonia (1 mL) and Raney nickel (3.2 g, 54.37 mmol, 6 eq) were added. The solution was stirred at 25° C. for 5 h under hydrogen atmosphere. After the reaction was completed, as detected by LC-MS, the mixture was filtered. The filtrate was concentrated by rotary evaporation to give the product (1.4 g, 68.7%). The crude product was used directly in the next step. LC-MS: [M+H]⁺=225.10.

### Step 4: Preparation of 2-chloro-N-(3-chloro-4-(pyrrolidin-1-ylmethyl)benzyl)-3-nitroquinoline-4-amine

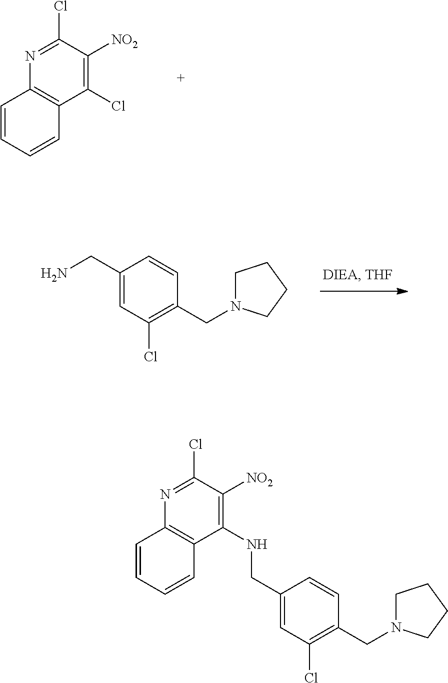 [SMILES: O=[N+]([O-])c1c(Cl)nc2ccccc2c1Cl ; NCc1ccc(CN2CCCC2)c(Cl)c1 ; O=[N+]([O-])c1c(Cl)nc2ccccc2c1NCc1ccc(CN2CCCC2)c(Cl)c1]

The compound 2,4-dichloro-3-nitroquinoline (1.8 g, 7.41 mmol, 1 eq) was dissolved in tetrahydrofuran (15 mL). N,N-diisopropylethylamine (1.9 g, 14.81 mmol, 2 eq) was added, and then a solution of (3-chloro-4-(pyrrolidin-1-ylmethyl)phenyl)methylamine (1.8 g, 8.15 mmol, 1.1 eq) in tetrahydrofuran (3 mL) was added dropwise. The solution was stirred at 25° C. for 2 h. After the reaction was completed, as detected by LC-MS, the reaction mixture was concentrated by rotary evaporation and was separated and purified by flash chromatography (DCM:MeOH=1:0 to 20:1) to give the product (2.2 g, 68.9%). LC-MS: [M+H]⁺=431.10.

### Step 5: Preparation of N⁴-(3-chloro-4-(pyrrolidin-1-ylmethyl)benzyl)-N²,N²-bis(4-methoxybenzyl)-3-nitroquinolin-2,4-diamine

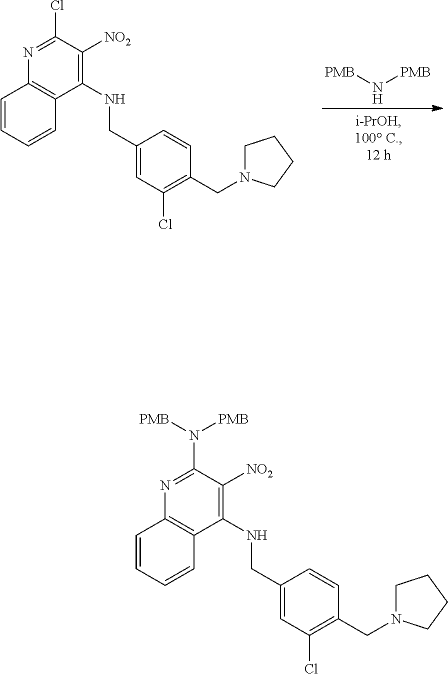

The compound 2-chloro-N-(3-chloro-4-(pyrrolidin-1-ylmethyl)benzyl)-3-nitroquinolin-4-amine (2.0 g, 4.64 mmol, 1 eq) was dissolved in isopropanol (20 mL). Bis(4-methoxybenzyl)amine (2.4 g, 9.27 mmol, 2 eq) was added. The reaction mixture was reacted at 100° C. for 48 h. After the reaction was completed, as detected by LC-MS, the reaction mixture was cooled to room temperature and concentrated by rotary evaporation. The crude product was purified using a C18 column (0.01% aqueous formic acid:CAN=70:30 to 50:50) to give the product (1.6 g, 52.9%). LC-MS: [M+H]⁺=652.50.

### Step 6: Preparation of N⁴-(3-chloro-4-(pyrrolidin-1-ylmethyl)benzyl)-N²,N²-bi s(4-methoxybenzyl)quinolin-2,3,4-triamine

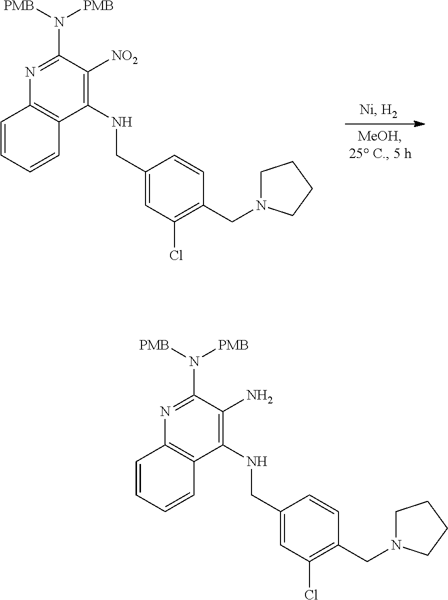

The compound N⁴-(3-chloro-4-(pyrrolidin-1-ylmethyl)benzyl)-N²,N²-bis(4-methoxybenzyl)-3-nitroquinolin-2,4-diamine (530 mg, 0.81 mmol, 1 eq) was dissolved in methanol (10 mL). Raney nickel (286 mg, 4.87 mmol, 6 eq) was added. The solution was reacted at 25° C. for 5 h under hydrogen atmosphere. After the reaction was completed, as detected by LC-MS, the mixture was filtered. The filter cake was washed with methanol. The filtrate was concentrated to give the product (490 mg, 96.9%). LC-MS: [M+H]⁺=622.30.

### Step 7: Preparation of 4-(bis(4-methoxybenzyl)amino)-1-(3-chloro-4-(pyrrolidin-1-ylmethyl)benzyl)-1,3-dihydro-2H-imidazo[4,5-c]quinolin-2-one

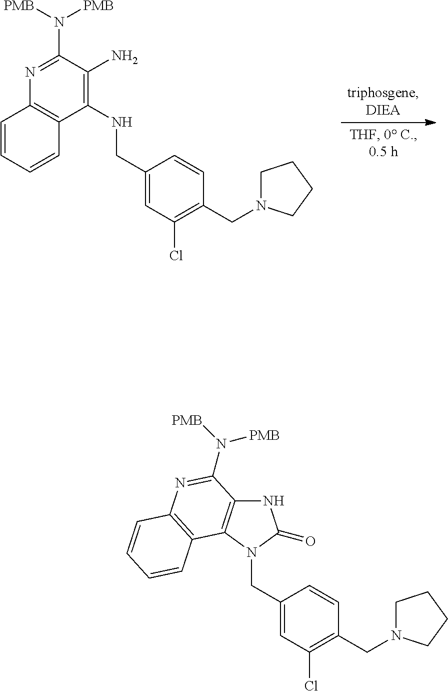

The compound N⁴-(3-chloro-4-(pyrrolidin-1-ylmethyl)benzyl)-N²,N²-bis(4-methoxybenzyl)quinolin-2,3,4-triamine (490 mg, 0.788 mmol, 1 eq) was dissolved in tetrahydrofuran (10 mL). N,N-diisopropylethylamine (203 mg, 1.575 mmol, 2 eq) was added, and then a solution of bis(trichloromethyl) carbonate (93 mg, 0.32 mmol, 0.4 eq) in tetrahydrofuran (1 mL) was added dropwise at 0° C. The solution was reacted at 0° C. for 0.5 h. After the reaction was completed, as detected by LC-MS, water (10 mL) was added to the reaction system. The mixture was extracted with ethyl acetate (20 mL×3). The organic phases were combined, dried over anhydrous sodium sulfate, filtered and concentrated to give the product (480 mg, 94%). The crude product was used directly in the next step. LC-MS: [M+H]⁺=648.40.

### Step 8: Preparation of 4-amino-1-(3-chloro-4-(pyrrolidin-1-ylmethyl)benzyl)-1,3-dihydro-2H-imidazo[4,5-c]quinolin-2-one

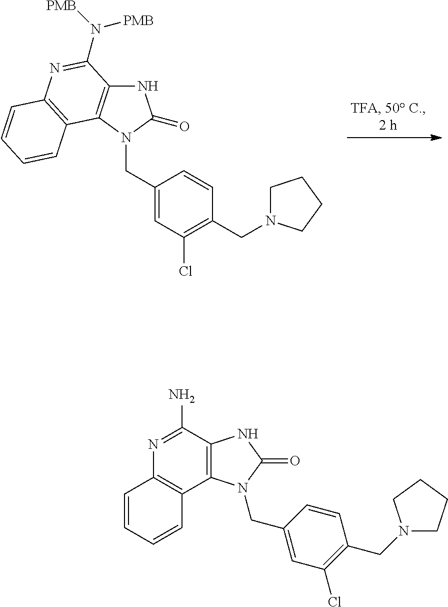

The compound 4-(bis(4-methoxybenzyl)amino)-1-(3-chloro-4-(pyrrolidin-1-ylmethyl)benzyl)-1,3-dihydro-2H-imidazo[4,5-c]quinolin-2-one (200 mg, 0.31 mmol, 1 eq) was dissolved in trifluoroacetic acid (5 mL). The solution was stirred at 50° C. for 1 h. After the reaction was completed, as detected by LC-MS, the reaction mixture was cooled to room temperature and concentrated by rotary evaporation. The crude product was separated and purified by Prep-HPLC (0.1% aqueous HCl solution, ACN) to give the target compound (60 mg, 47.7%). LC-MS: [M+H]⁺=408.30; ¹H NMR (400 MHz, DMSO-d₆): δ 14.25 (s, 1H), 12.58 (s, 1H), 10.94 (s, 1H), 8.81 (s, 2H), 8.00-7.81 (m, 3H), 7.70 (t, J=7.7 Hz, 1H), 7.64 (s, 1H), 7.51-7.34 (m, 2H), 5.64 (s, 2H), 4.51 (d, J=5.4 Hz, 2H), 3.45 (m, 2H), 3.24-3.01 (m, 2H), 2.20-1.81 (m, 4H).

### Example 33: Preparation of 4-amino-1-(4-(pyrrolidin-1-ylmethyl)phenyl)amino)-1,3-dihydro-2H-imidazo[4,5-c]quinolin-2-one

### Step 1: Preparation of 1-(4-iodobenzyl)pyrrolidine

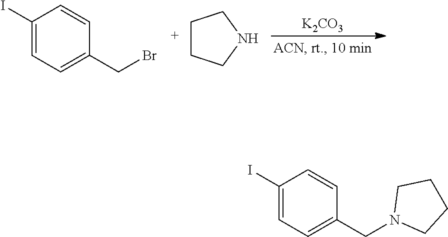

Tetrahydropyrrole (3.6 g, 50.51 mmol, 3 eq.) is dissolved in acetonitrile (100 mL). Potassium carbonate (7.0 g, 50.51 mmol, 3 eq.) was added to the reaction system, and a solution of 4-iodobenzyl bromide (5.0 g, 16.84 mmol, 1 eq.) in acetonitrile (100 mL) was added dropwise. The reaction mixture was stirred at 25° C. for 10 min. After the reaction was completed, as detected by LC-MS, the mixture was filtered to remove the solid particles, and the filtrate was concentrated by rotary evaporation to give the product (4.6 g, 95%). LC-MS: [M+H]⁺=287.90.

### Step 2: Preparation of tert-butyl 1-(4-(pyrrolidin-1-ylmethyl)phenyl)hydrazine-1-carboxylate

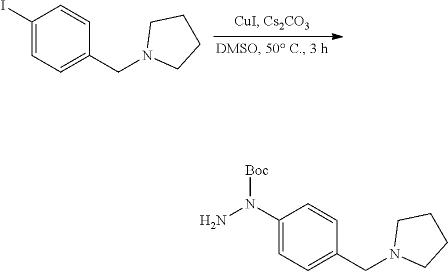 [SMILES: Ic1ccc(CN2CCCC2)cc1 ; CC(C)(C)OC(=O)N(N)c1ccc(CN2CCCC2)cc1]

The compound 1-(4-iodobenzyl)pyrrolidine (4.5 g, 15.67 mmol, 1 eq.) and tert-butyl hydrazine carboxylate (4.1 g, 31.34 mmol, 2 eq.) were dissolved in dimethyl sulfoxide (50 mL), and then copper(I) iodide (596 mg, 3.13 mmol, 0.2 eq.), cesium carbonate (10.2 g, 31.34 mmol, 2 eq.), and L-hydroxyproline (822 mg, 6.27 mmol, 0.4 eq.) were added sequentially. The solution was stirred at 50° C. for 3 h. After the reaction was completed, as detected by LC-MS, the reaction mixture was cooled to room temperature and filtered. The filtrate was separated and purified using a C18 column (0.1% aqueous NH₃ solution:MeOH)=9:91) to give the product (2.3 g, 50.4%). LC-MS: [M+H]⁺=292.10.

### Step 3: Preparation of tert-butyl 2-(2-chloro-3-nitroquinolin-4-yl)-1-(4-(pyrrolidin-1-ylmethyl)phenyl)hydrazine-1-carboxylate

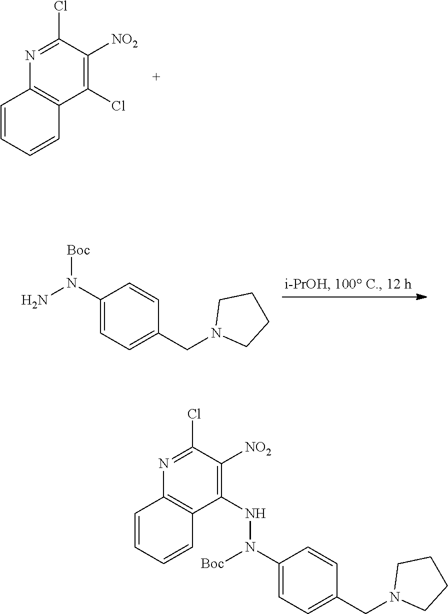 [SMILES: O=[N+]([O-])c1c(Cl)nc2ccccc2c1Cl ; CC(C)(C)OC(=O)N(N)c1ccc(CN2CCCC2)cc1 ; CC(C)(C)OC(=O)N(Nc1c([N+](=O)[O-])c(Cl)nc2ccccc12)c1ccc(CN2CCCC2)cc1]

The compound 2,4-dichloro-3-nitroquinoline (1 g, 4.11 mmol, 1 eq.) and tert-butyl 1-(4-(pyrrolidin-1-ylmethyl)phenyl)hydrazine-1-carboxylate (1.2 g, 4.11 mmol, 1 eq.) were dissolved in isopropanol (4 mL). The solution was stirred at 100° C. for 12 h. After the reaction was completed, as detected by LC-MS, the reaction mixture was cooled to room temperature and concentrated by rotary evaporation. The crude product was separated and purified by flash chromatography (DCM:MeOH=15:1) to give the product (800 mg, 39%). LC-MS: [M+H]⁺=498.31.

### Step 4: Preparation of tert-butyl 2-(3-amino-2-chloroquinolin-4-yl)-1-(4-(pyrrolidin-1-ylmethyl)phenyl)hydrazine-1-carboxylate

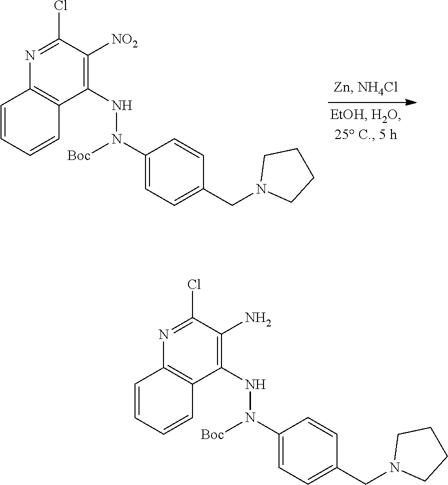 [SMILES: CC(C)(C)OC(=O)N(Nc1c([N+](=O)[O-])c(Cl)nc2ccccc12)c1ccc(CN2CCCC2)cc1 ; CC(C)(C)OC(=O)N(Nc1c(N)c(Cl)nc2ccccc12)c1ccc(CN2CCCC2)cc1]

The compound tert-butyl 2-(2-chloro-3-nitroquinolin-4-yl)-1-(4-(pyrrolidin-1-ylmethyl)phenyl)hydrazine-1-carboxylate (700 mg, 1.41 mmol, 1 eq.) was dissolved in ethanol (10 mL) and water (10 mL), and then ammonium chloride (752 mg, 14.06 mmol, 10 eq.) and zinc powder (919 mg, 14.06 mmol, 10 eq.) were added. The solution was stirred at 25° C. for 5 h. After the reaction was completed, as detected by LC-MS, the mixture was filtered, and the filtrate was extracted with ethyl acetate (100 mL×3). The organic phases were dried over anhydrous sodium sulfate, filtered and concentrated. The crude product was separated and purified by flash chromatography (DCM:MeOH=100:0 to 90:10) to give the product (540 mg, 82.1%). LC-MS: [M+H]⁺=468.3.

### Step 5: Preparation of tert-butyl 2-(3-amino-2-chloroquinolin-4-yl)-1-(4-(chloromethyl)phenyl)hydrazine-1-carboxylate

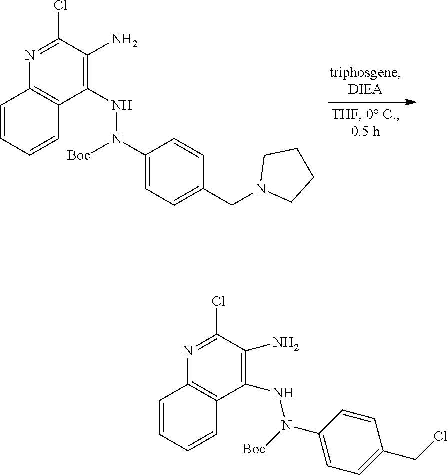 [SMILES: CC(C)(C)OC(=O)N(Nc1c(N)c(Cl)nc2ccccc12)c1ccc(CN2CCCC2)cc1 ; CC(C)(C)OC(=O)N(Nc1c(N)c(Cl)nc2ccccc12)c1ccc(CCl)cc1]

The compound tert-butyl 2-(3-amino-2-chloroquinolin-4-yl)-1-(4-(pyrrolidin-1-ylmethyl)phenyl)hydrazine-1-carboxylate (300 mg, 0.64 mmol, 1 eq.) was dissolved in tetrahydrofuran (5 mL). N,N-diisopropylethylamine (166 mg, 1.28 mmol, 2 eq.) was added. A solution of triphosgene (76 mg, 0.25 mmol, 0.4 eq.) in tetrahydrofuran (1 mL) was added dropwise at 0° C. The reaction mixture was stirred at 0° C. for 0.5 h. After the reaction was completed, as detected by LC-MS, the reaction mixture was quenched with saturated sodium bicarbonate (10 mL), and extracted with ethyl acetate (20 mL×3). The organic phases were dried over anhydrous sodium sulfate, filtered and concentrated to give a crude product (280 mg, 100%). LC-MS: [M+H]+=432.90.

### Step 6: Preparation of tert-butyl (4-chloro-2-oxo-2,3-dihydro-1H-imidazo[4,5-c]quinolin-1-yl)(4-(chloromethyl)phenyl)carbamate

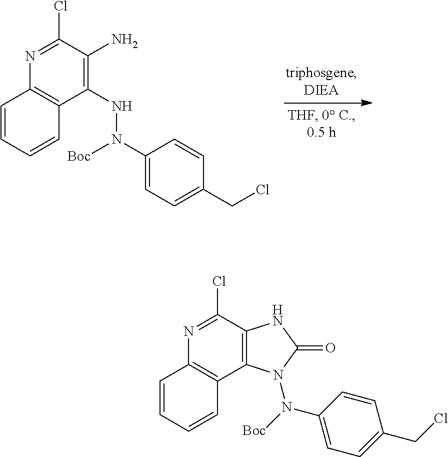 [SMILES: CC(C)(C)OC(=O)N(Nc1c(N)c(Cl)nc2ccccc12)c1ccc(CCl)cc1 ; CC(C)(C)OC(=O)N(c1ccc(CCl)cc1)n1c(=O)[nH]c2c(Cl)nc3ccccc3c21]

The compound tert-butyl 2-(3-amino-2-chloroquinolin-4-yl)-1-(4-(chloromethyl)phenyl)hydrazine-1-carboxylate (260 mg, 0.60 mmol, 1 eq.) was dissolved in tetrahydrofuran. N,N-diisopropylethylamine (155 mg, 1.20 mmol, 2 eq.) was added. A solution of triphosgene (71 mg, 0.24 mmol, 0.4 eq.) in tetrahydrofuran (2 mL) was added dropwise at 0° C. The reaction mixture was stirred at 0° C. for 0.5 h. After the reaction was completed, as detected by LC-MS, the reaction mixture was quenched with saturated sodium bicarbonate solution (10 mL), and extracted with ethyl acetate (20 mL×3). The organic phases were dried over anhydrous sodium sulfate, filtered and concentrated to give a crude product (260 mg, 94.3%). LC-MS: [M+H]⁺=458.80.

### Step 7: Preparation of tert-butyl (4-chloro-2-oxo-2,3-dihydro-1H-imidazo[4,5-c]quinolin-1-yl)(4-(pyrrolidin-1-ylmethyl)phenyl)carbamate

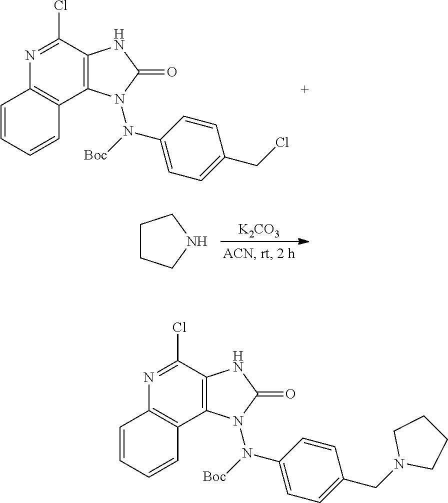

The compound tetrahydropyrrole (121 mg, 1.70 mmol, 3 eq.) was dissolved in acetonitrile (3 mL). Potassium carbonate (235 mg, 1.70 mmol, 3 eq.) was added, and then a solution of tert-butyl (4-chloro-2-oxo-2,3-dihydro-1H-imidazo[4,5-c]quinolin-1-yl)(4-(chloromethyl)phenyl)carbamate (260 mg, 0.57 mmol, 1 eq.) in acetonitrile (1 mL) was added dropwise. The reaction mixture was stirred at 25° C. for 2 h. After the reaction was completed, as detected by LC-MS, the reaction mixture was concentrated. The crude product was separated and purified by flash chromatography (DCM:MeOH=100:0 to 92:8) to give the product (80 mg, 28.6%). LC-MS: [M+H]⁺=494.30.

### Step 8: Preparation of tert-butyl (4-amino-2-oxo-2,3-dihydro-1H-imidazo[4,5-c]quinolin-1-yl)(4-(pyrrolidin-1-ylmethyl)phenyl)carbamate

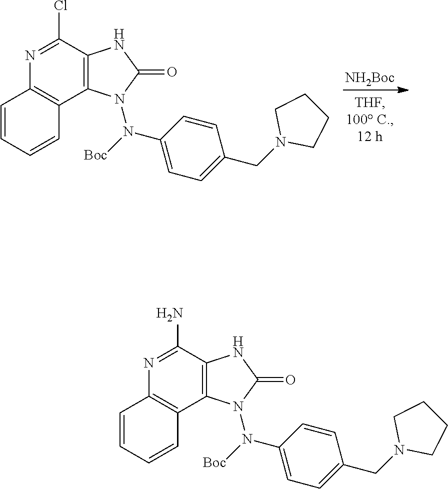 [SMILES: CC(C)(C)OC(=O)N(c1ccc(CN2CCCC2)cc1)n1c(=O)[nH]c2c(Cl)nc3ccccc3c21 ; CC(C)(C)OC(=O)N(c1ccc(CN2CCCC2)cc1)n1c(=O)[nH]c2c(N)nc3ccccc3c21 ; "NH2Boc"]

The compound tert-butyl (4-chloro-2-oxo-2,3-dihydro-1H-imidazo[4,5-c]quinolin-1-yl)(4-(pyrrolidin-1-ylmethyl)phenyl)carbamate (70 mg, 0.14 mmol, 1 eq.) was dissolved in tetrahydrofuran (5 mL), methanesulfonato2-dicyclohexylphosphino-3,6-dimethoxy-2′-4′-6′-tri-i-propyl-1,1′-bipheny)(2′-amino-1,1′-biphenyl-2-yl)palladium(II)) (12 mg, 0.01 mmol, 0.1 eq.), tert-butyl carbamate (166 mg, 1.42 mmol, 10 eq.), and cesium carbonate (139 mg, 0.43 mmol, 3 eq.) were added. The solution was reacted at 100° C. for 12 h in a sealed tube. After the reaction was completed, as detected by LC-MS, the reaction mixture was cooled to room temperature. Diluted hydrochloric acid (1 M, 5 mL) was added. The mixture was stirred for 1 h, and extracted with ethyl acetate (10 mL×3). The aqueous phase was removed by rotary evaporation to give a crude product (70 mg), which was used directly in the next step. LC-MS: [M+H]⁺=475.00.

### Step 9: Preparation of 4-amino-1-((4-(pyrrolidin-1-ylmethyl)phenyl)amino)-1,3-dihydro-2H-imidazo[4,5-c]quinolin-2-one

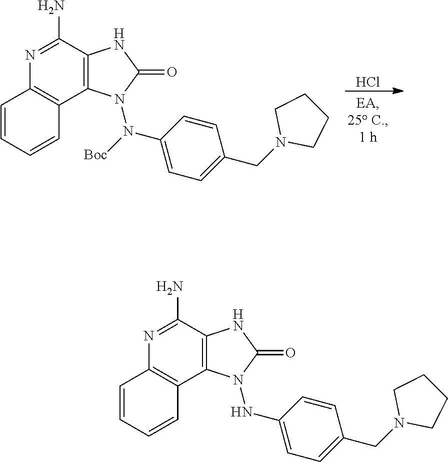 [SMILES: CC(C)(C)OC(=O)N(c1ccc(CN2CCCC2)cc1)n1c(=O)[nH]c2c(N)nc3ccccc3c21 ; Cl ; Nc1nc2ccccc2c2c1[nH]c(=O)n2Nc1ccc(CN2CCCC2)cc1]

The compound tert-butyl (4-amino-2-oxo-2,3-dihydro-1H-imidazo[4,5-c]quinolin-1-yl)(4-(pyrrolidin-1-ylmethyl)phenyl)carbamate (70 mg, 0.14 mmol, 1 eq.) was dissolved in a 3 mol/L solution of HCl in ethyl acetate (5 mL). The mixture was reacted at 25° C. for 0.5 h. After the reaction was completed, as detected by LC-MS, the reaction mixture was concentrated. The crude product was separated and purified by Prep-HPLC (0.1% aqueous HCl solution, MeCN) to give the product (6.0 mg, 10.9%). LC-MS: [M+H]⁺=375.30; ¹H NMR (400 MHz, methanol-d₄): δ 8.53 (d, J=8.4 Hz, 1H), 7.74 (q, J=8.0, 7.3 Hz, 2H), 7.44 (m, 3H), 6.92 (d, J=8.4 Hz, 2H), 4.28 (s, 2H), 3.53-3.41 (m, 2H), 3.20-3.09 (m, 2H), 2.24-2.08 (m, 2H), 2.06-1.89 (m, 2H).

### Example 34: Preparation of 8-amino-6-butoxy-3-(4-(pyrrolidin-1-ylmethyl)benzyl)-3,4-dihydropyrimido[5,4-d]pyrimidin-2(1H)-one

### Step 1: Preparation of tert-butyl (4-(bis(4-methoxybenzyl)amino)-2-butoxy-6-((4-(pyrrolidin-1-ylmethyl)benzyl)amino)methyl)pyrimidin-5-yl)carbamate

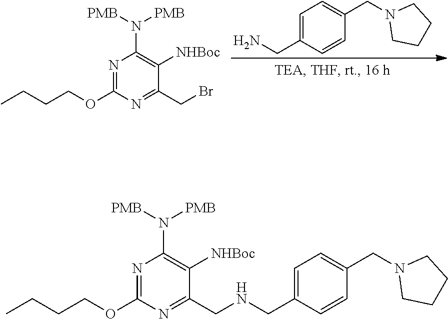

The compound tert-butyl (4-(bis(4-methoxybenzyl)amino)-6-(bromomethyl)-2-butoxypyrimidin-5-yl)carbamate (0.3 g, 0.49 mmol, 1 eq) was dissolved in tetrahydrofuran (3 mL). N,N-diisopropylethylamine (95 mg, 0.73 mmol, 1.5 eq) and a solution of the compound (4-(pyrrolidin-1-ylmethyl)phenyl)methylamine (0.12 g, 0.63 mmol, 1.3 eq) in tetrahydrofuran (1 mL) were added sequentially dropwise. After the dropwise addition, the reaction mixture was stirred at 25° C. for 16 h. After the reaction was completed, as detected by LC-MS, the reaction mixture was concentrated by rotary evaporation. The crude product was separated and purified by flash chromatography (DCM:MeOH=20:1) to give the target compound (0.2 g, 56.6%). LC-MS: [M+H]⁺=725.30.

### Step 2: Preparation of 8-(bis(4-methoxybenzyl)amino)-6-butoxy-3-(4-(pyrrolidin-1-ylmethyl)benzyl)-3,4-dihydropyrimido[5,4-d]pyrimidin-2(1H)-one

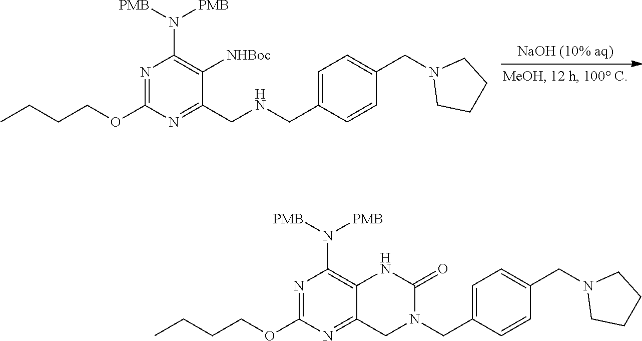

The compound tert-butyl (4-(bis(4-methoxybenzyl)amino)-2-butoxy-6-((4-(pyrrolidin-1-ylmethyl)benzyl)amino)methyl)pyrimidin-5-yl)carbamate (200 mg, 0.28 mmol, 1 eq) was dissolved in methanol (10 mL), and then 10% NaOH solution (2 mL) was added dropwise. The mixture was stirred at 100° C. for 12 h. After the reaction was completed, as detected by LC-MS, the reaction mixture was cooled to room temperature, water (10 mL) was added to the reaction system, and then the mixture was extracted with dichloromethane (20 mL×3). The organic phases were combined, dried over anhydrous ammonium sulfate, filtered and concentrated to give a crude target compound (200 mg), which was used directly in the next step. LC-MS: [M+H]⁺=651.61.

### Step 3: Preparation of 8-amino-6-butoxy-3-(4-(pyrrolidin-1-ylmethyl)benzyl)-3,4-dihydropyrimido[5,4-d]pyrimidin-2(1H)-one

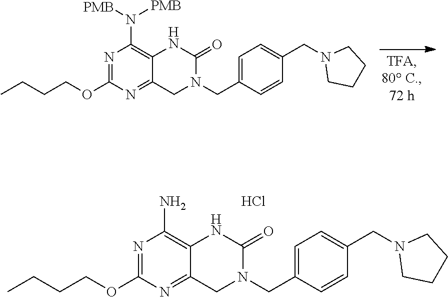

The crude product of the compound 8-(bis(4-methoxybenzyl)amino)-6-butoxy-3-(4-(pyrrolidin-1-ylmethyl)benzyl)-3,4-dihydropyrimido[5,4-d]pyrimidin-2(1H)-one (200 mg) was dissolved in trifluoroacetic acid (5 mL). The solution was stirred at 80° C. for 72 h. After the reaction was completed, as detected by LC-MS, the reaction mixture was cooled to room temperature and concentrated by rotary evaporation. The crude product was separated and purified by Prep-HPLC (0.1% aqueous HCl solution, MeCN) to give the product (24 mg, 19%). LC-MS: [M+H]⁺=411.21; ¹H NMR (400 MHz, DMSO-d₆): δ 10.62 (s, 1H), 8.81 (s, 1H), 7.57 (d, J=8.0 Hz, 2H), 7.39 (d, J=8.0 Hz, 2H), 4.56 (s, 2H), 4.32 (d, J=5.8 Hz, 2H), 4.20 (s, 2H), 4.14 (t, J=6.5 Hz, 2H), 3.33-3.31 (m, 2H), 3.12-2.96 (m, 2H), 2.08-1.92 (m, 2H), 1.87 (m, 2H), 1.72-1.55 (m, 2H), 1.36 (m, 2H), 0.89 (t, J=7.4 Hz, 3H).

### Example 35: Preparation of 8-amino-3-benzyl-6-butoxy-3,4-dihydropyrimido[5,4-d]pyrimidin-2(1H)-one

### Step 1: Preparation of tert-butyl (4-((benzylamino)methyl)-6-(bis(4-methoxybenzyl)amino)-2-butoxypyrimidin-5-yl)carbamate

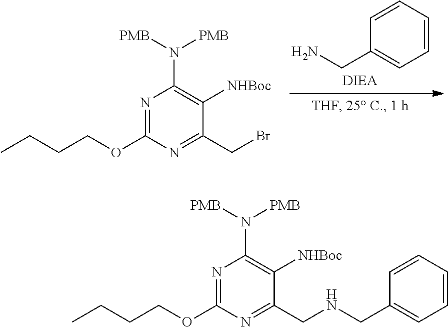

Tert-butyl (4-(bis(4-methoxybenzyl)amino)-6-(bromomethyl)-2-butoxypyrimidin-5-yl)carbamate (300 mg, 0.49 mmol, 1 eq) was dissolved in tetrahydrofuran (5 mL). N,N-diisopropylethylamine (189 mg, 1.46 mmol, 3 eq) was added dropwise, and then benzylamine (78 mg, 0.73 mmol, 1.5 eq) was added dropwise. After the dropwise addition, the mixture was stirred at 25° C. for 1 h. After the reaction was completed, as detected by LC-MS, water (10 mL) was added to the reaction system, and then the mixture was extracted with ethyl acetate (20 mL×2). The organic phases were combined, dried over anhydrous sodium sulfate, filtered and concentrated. The crude product was separated and purified by flash chromatography (EA:PE=0:100 to 50:50) to give the product (135 mg, 43.2%). LC-MS: [M+H]⁺=642.31.

### Step 2: Preparation of 3-benzyl-8-(bis(4-methoxybenzyl)amino)-6-butoxy-3,4-dihydropyrimido[5,4-d]pyrimidin-2(1H)-one

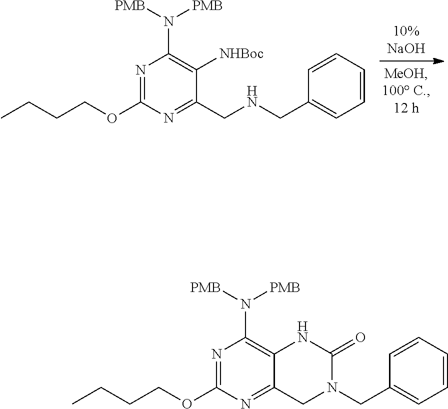

Tert-butyl (4-((benzylamino)methyl)-6-(bis(4-methoxybenzyl)amino)-2-butoxypyrimidin-5-yl)carbamate (135 mg, 0.21 mmol, 1 eq) was dissolved in methanol (10 mL), and then 10% NaOH solution (3 mL) was added dropwise. The solution was stirred at 100° C. for 12 h. After the reaction was completed, as detected by LC-MS, the reaction mixture was cooled to room temperature, H₂O (10 mL) was added to the reaction system, and then the mixture was extracted with dichloromethane (3×10 mL). The organic phases were combined, dried over anhydrous sulfuric acid, filtered and concentrated to give a crude product (135 mg), which was used directly in the next step. LC-MS: [M+H]⁺=568.11.

### Step 3: Preparation of 8-amino-3-benzyl-6-butoxy-3,4-dihydropyrimido[5,4-d]pyrimidin-2(1H)-one

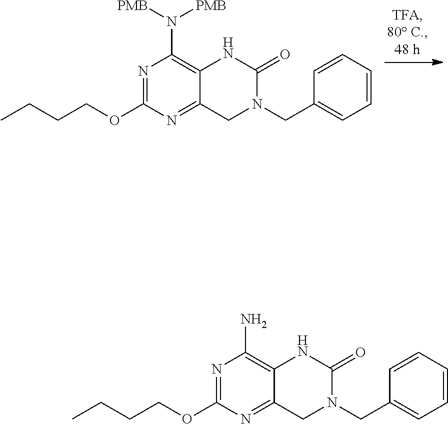

The crude product of 3-benzyl-8-(bis(4-methoxybenzyl)amino)-6-butoxy-3,4-dihydropyrimido[5,4-d]pyrimidin-2(1H)-one (100 mg) was dissolved in trifluoroacetic acid (5 mL). The solution was stirred at 80° C. for 48 h. After the reaction was completed, as detected by LC-MS. the reaction mixture was cooled to room temperature and concentrated by rotary evaporation. The crude product was separated and purified by Prep-HPLC (0.1% aqueous HCl solution, MeCN) to give the product (16 mg, purity: 99%, 19%). LC-MS: [M+H]⁺=328.81; ¹H NMR (400 MHz, DMSO-d₆): δ 8.96 (s, 1H), 7.99 (s, 1H), 7.34 (m, 5H), 4.55 (s, 2H), 4.23 (m, 4H), 1.64 (m, 2H), 1.36 (m, 2H), 0.90 (t, J=7.4 Hz, 3H).

### Example 36: Preparation of 8-amino-6-butoxy-3-((5-(pyrrolidin-1-ylmethyl)thien-2-yl)methyl)-3,4-dihydropyrimido[5,4-d]pyrimidin-2(1H)-one

### Step 1: Preparation of tert-butyl (4-(bis(4-methoxybenzyl)amino)-2-butoxy-6-((((5-(pyrrolidin-1-ylmethyl)thien-2-yl)methyl)amino)methyl)pyrimidin-5-yl)carbamate

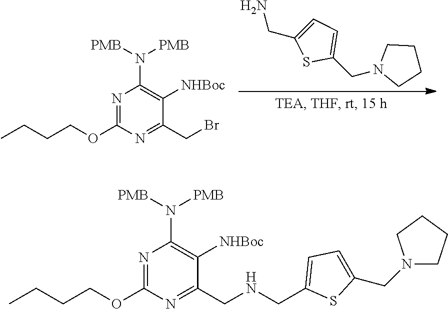

The starting material tert-butyl (4-(bis(4-methoxybenzyl)amino)-6-(bromomethyl)-2-butoxypyrimidin-5-yl)carbamate (240 mg, 0.39 mmol, 1 eq.) was dissolved in tetrahydrofuran (1.5 mL), and then triethylamine (118 mg, 1.17 mmol, 3 eq.) was added. The starting material (5-(pyrrolidin-1-ylmethyl)thien-2-yl)methylamine (92 mg, 0.47 mmol, 1.2 eq.) was dissolved in tetrahydrofuran (1 mL), and then the mixture was added dropwise to the reaction mixture. After the dropwise addition, the mixture was stirred at room temperature for 16 h. The reaction mixture was concentrated to remove the tetrahydrofuran to give a crude product, which was separated and purified by flash chromatography (DCM:MeOH=100:0 to 94:6) to give the target compound (140 mg, 49.1%). LC-MS: [M+H]⁺=731.51.

### Step 2: Preparation of 8-(bis(4-methoxybenzyl)amino)-6-butoxy-3-((5-(pyrrolidin-1-ylmethyl)thien-2-yl)methyl)-3,4-dihydropyrimido[5,4-d]pyrimidin-2(1H)-one

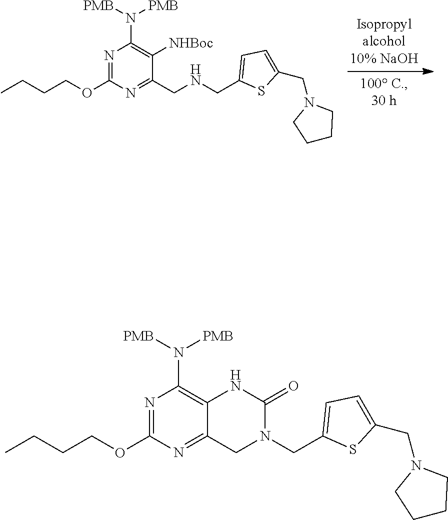

The starting material tert-butyl (4-(bis(4-methoxybenzyl)amino)-2-butoxy-6-((((5-(pyrrolidin-1-ylmethyl)thien-2-yl)methyl)amino)pyrimidin-5-yl)methyl)carbamate (120 mg, 0.16 mmol, 1 eq.) was dissolved in a mixed solution of isopropanol (10 mL) and 10% aqueous sodium hydroxide solution (2 mL), and then the reaction mixture was stirred at 100° C. for 30 h. After the reaction was completed, as detected by LC-MS, water (8 mL) was added to the reaction mixture. The mixture was extracted with ethyl acetate (15 mL×3). The organic phases were combined, dried over anhydrous sodium sulfate, filtered and concentrated to give a crude product (100 mg, 92.7%), which was used directly in the next step. LC-MS: [M+H]⁺=657.31.

### Step 3: Preparation of 8-amino-6-butoxy-3-((5-(pyrrolidin-1-ylmethyl)thien-2-yl)methyl)-3,4-dihydropyrimido[5,4-d]pyrimidin-2(1H)-one

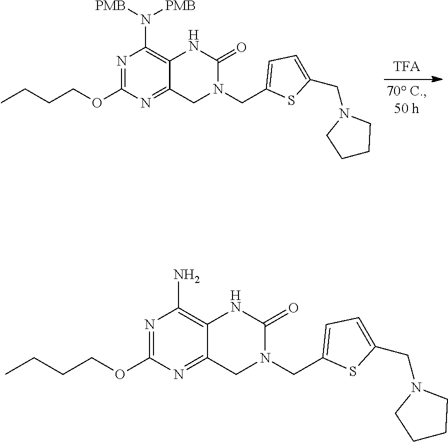

The compound 8-(bis(4-methoxybenzyl)amino)-6-butoxy-3-((5-(pyrrolidin-1-ylmethyl)thien-2-yl)methyl)-3,4-dihydropyrimido[5,4-d]pyrimidin-2(1H)-one (100 mg, 0.15 mmol, 1 eq.) was dissolved in trifluoroacetic acid (10 mL), and then the reaction mixture was heated and stirred at 70° C. for 50 h. The mixture was concentrated to remove the trifluoroacetic acid to give a crude product, which was separated and purified by Prep-HPLC (0.05% aqueous HCl solution, MeCN) to give the target compound (24.3 mg, 38.3%). LC-MS: [M+H]⁺=417.21; ¹H NMR (400 MHz, MeOH-d₄): δ 7.29-7.22 (m, 1H), 7.18-7.14 (m, 1H), 4.79 (s, 2H), 4.59 (d, J=6.8 Hz, 2H), 4.48 (t, J=6.4 Hz, 4H), 3.59-3.51 (m, 2H), 3.25-3.17 (m, 2H), 2.22-2.13 (m, 2H), 2.08-1.97 (m, 2H), 1.81-1.74 (m, 2H), 1.52-1.43 (m, 2H), 0.98 (t, J=7.6 Hz, 3H).

### Example 37: Preparation of 8-amino-6-butoxy-3-(5-(pyrrolidin-1-yl)pentyl)-3,4-dihydropyrimido[5,4-d]pyrimidin-2(1H)-one

### Step 1: Preparation of tert-butyl (4-(bis(4-methoxybenzyl)amino)-2-butoxy-6-(((5-(pyrrolidin-1-yl)pentyl)amino)methyl)pyrimidin-5-yl)carbamate

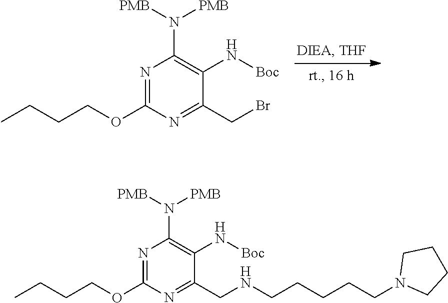

Tert-butyl (4-(bis(4-m ethoxybenzyl)amino)-6-(bromomethyl)-2-butoxypyrimidin-5-yl)carbamate (0.3 g, 0.49 mmol, 1 eq.) was dissolved in tetrahydrofuran (5 mL). 5-(pyrrolidin-1-yl)pentan-1-amine (0.11 g, 0.73 mmol, 1.5 eq.) and N,N-diisopropylethylamine (0.31 g, 2.44 mmol, 5 eq.) were added sequentially to the reaction system. The mixture was reacted at room temperature for 16 h. After the reaction was completed, water (20 mL) was added to the reaction system. The mixture was extracted with ethyl acetate (15 mL×3). The organic phases were combined, washed with saturated brine (50 mL), dried over anhydrous sodium sulfate, filtered and concentrated. The crude product was separated and purified by flash chromatography (DCM:MeOH=3:1) to give the target compound (0.1 g, 29.7%). LC-MS: [M+H]⁺=691.41.

### Step 2: Preparation of 8-(bis(4-methoxybenzyl)amino)-6-butoxy-3-(5-(pyrrolidin-1-yl)pentyl)-3,4-dihydropyrimido[5,4-d]pyrimidin-2(1H)-one

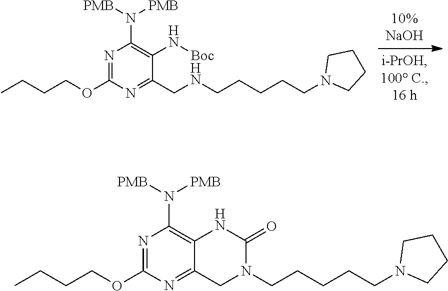

Tert-butyl (4-(bis(4-methoxybenzyl)amino)-2-butoxy-6-(((5-(pyrrolidin-1-yl)pentyl)amino)methyl)pyrimidin-5-yl)carbamate (0.1 g, 0.14 mmol, 1 eq.) was dissolved in isopropanol (5 mL) and NaOH (10%, 1 mL). The mixture was reacted at 100° C. for 16 h. After the reaction was completed, water (20 mL) was added to the reaction system. The mixture was extracted with ethyl acetate (15 mL×3). The organic phases were combined, washed with saturated brine (20 mL), dried over anhydrous sodium sulfate, filtered and concentrated to give a crude product (0.08 g, 89.6%), which was used directly in the next step. LC-MS: [M+H]⁺=617.31.

### Step 3: Preparation of 8-amino-6-butoxy-3-(5-(pyrrolidin-1-yl)pentyl)-3,4-dihydropyrimido[5,4-d]pyrimidin-2(1H)-one

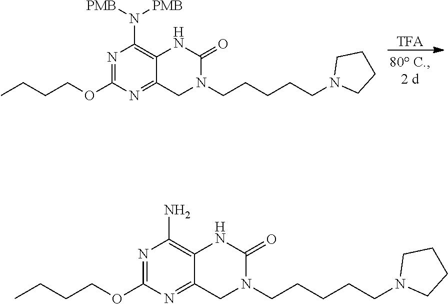

8-(bis(4-methoxybenzyl)amino)-6-butoxy-3-(5-(pyrrolidin-1-yl)pentyl)-3,4-dihydropyrimido[5,4-d]pyrimidin-2(1H)-one (0.08 g, 0.13 mmol, 1 eq.) was dissolved in trifluoroacetic acid (3 mL). The reaction mixture was stirred at 80° C. for 48 h. After the reaction was completed, as detected by LC-MS, the reaction mixture was concentrated to give a crude product, which was separated and purified by Prep-HPLC (0.01% aqueous HCl solution, MeCN) to give the product (0.022 g, 45.1%). LC-MS: [M+H]⁺=377.11; ¹H NMR (400 MHz, DMSO-d₆): δ 10.18 (s, 1H), 8.60 (s, 1H), 4.30 (s, 2H), 4.17 (t, J=6.4 Hz, 2H), 3.52-3.45 (m, 2H), 3.31 (t, J=7.2 Hz, 2H), 3.11-3.04 (m, 2H), 3.00-2.89 (m, 2H), 2.02-1.96 (m, 2H), 1.96-1.82 (m, 2H), 1.72-1.60 (m, 4H), 1.59-1.52 (m, 2H), 1.43-1.35 (m, 2H), 1.33-1.26 (m, 2H), 0.91 (t, J=7.6 Hz, 3H).

### Example 38: Preparation of 8-amino-6-(ethylthio)-3-(3-(pyrrolidin-1-ylmethyl)benzyl)-3,4-dihydropyrimido[5,4-d]pyrimidin-2(1H)-one

### Step 1: Preparation of 2-(ethylthio)pyrimidine-4,6-diol

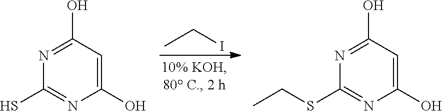

The starting material 2-mercaptopyrimidine-4,6-diol (45.0 g, 312.5 mmol, 1.0 eq) was dissolved in 10% aqueous potassium hydroxide solution (405 mL), and then iodoethane (53.6 g, 343.8 mmol, 1.1 eq) was added dropwise. The reaction mixture was stirred at 80° C. for 2 h. After the reaction was completed, as detected by LC-MS, the reaction mixture was cooled to room temperature, adjusted to pH=3 with 2 N HCl, and filtered. The solid was dried to give the target compound (40 g, 74.4%). LC-MS: [M+H]⁺=173.01.

### Step 2: Preparation of 2-(ethylthio)-5-nitropyrimidine-4,6-diol

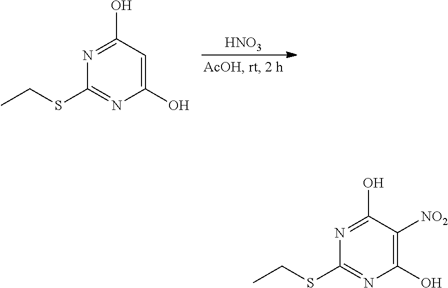

Acetic acid (90 mL) and fuming nitric acid (45 mL) were cooled to 5° C., and then 2-(ethylthio)pyrimidine-4,6-diol (30 g, 174.4 mmol, 1.0 eq) was added. The reaction mixture was stirred at 25° C. for 2 h. After the reaction was completed, as detected by LC-MS, water (300 mL) was added to the reaction mixture. The mixture was filtered and dried to give the target compound (22 g, 58.2%). LC-MS: [M+H]⁺=218.01.

### Step 3: Preparation of 4,6-dichloro-2-(ethylthio)-5-nitropyrimidine

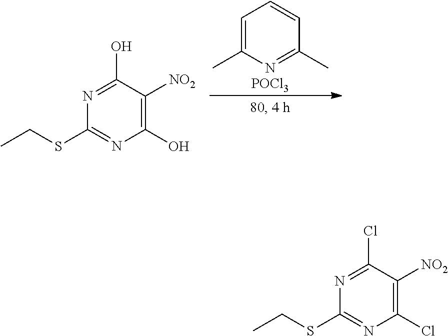

The starting material 2-(ethylthio)-5-nitropyrimidine-4,6-diol (33 g, 152.1 mmol, 1.0 eq) was dissolved in phosphorus oxychloride (100 mL), and then 2,6-lutidine (40.8 g, 380.2 mmol, 2.5 eq) was added at 0° C. The reaction mixture was stirred at 80° C. for 2 h. After the reaction was completed, as detected by LC-MS, water (500 mL) was poured into the reaction mixture, and then the mixture was extracted with dichloromethane (500 mL×3). The organic phases were combined, washed with saturated brine (500 mL), dried over anhydrous sodium sulfate, filtered and concentrated to give the target compound (35 g, 90.9%). The crude product was used directly in the next step.

### Step 4: Preparation of 6-chloro-2-(ethylthio)-N,N-bis(4-methoxybenzyl)-5-nitropyrimidin-4-amine

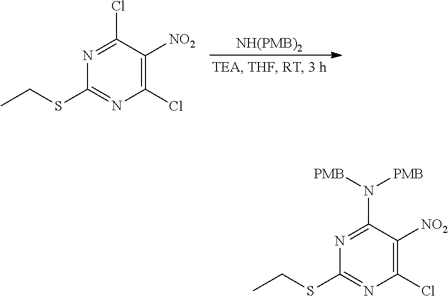

The starting material 4,6-dichloro-2-(ethylthio)-5-nitropyrimidine (15 g, 137.8 mmol, 1.0 eq) was dissolved in tetrahydrofuran (200 mL), and then triethylamine (10 g, 206.7 mmol, 1.5 eq) and bis(4-methoxybenzyl)amine (153 g, 137.8 mmol, 1.0 eq) was added. The reaction mixture was stirred at 25° C. for 16 h. After the reaction was completed, as detected by LC-MS, water (200 mL) was added to the reaction mixture. The mixture was extracted with ethyl acetate (200 mL×3). The organic phases were combined, dried over anhydrous sodium sulfate, filtered and concentrated to give the target compound (25 g, 89.3%). LC-MS: [M+H]⁺=475.01.

### Step 5: Preparation of 6-chloro-2-(ethanethiol)-N,N-bis(4-methoxybenzyl)pyrimidine-4,5-diamine

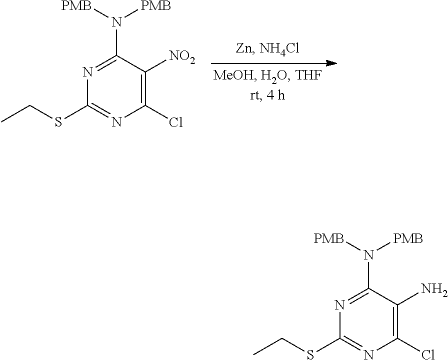

The compound 6-chloro-2-(ethylthio)-N,N-bis(4-methoxybenzyl)-5-nitropyrimidin-4-amine (20 g, 42.1 mmol, 1.0 eq) was dissolved in methanol (40 mL), water (40 mL), and tetrahydrofuran (80 mL), and then ammonium chloride (11.3 g, 211.0 mmol, 5.0 eq) and zinc powder (13.8 g, 211.0 mmol, 5.0 eq) were added. The reaction was stirred at 25° C. for 4 h. After the reaction was completed, as detected by LC-MS, the reaction mixture was filtered. Water (150 mL) was added to the filtrate, and then the mixture was extracted with ethyl acetate (200 mL×3). The organic phases were combined, washed with saturated brine (200 mL), dried over anhydrous sodium sulfate, filtered and concentrated to give a crude product, which was slurried with ethyl acetate and filtered to give the target compound (9.6 g, 51.3%). LC-MS: [M+H]⁺=377.11.

### Step 6: Preparation of methyl 5-amino-6-(bis(4-methoxybenzyl)amino)-2-(ethylthio)pyrimidine-4-carboxylate

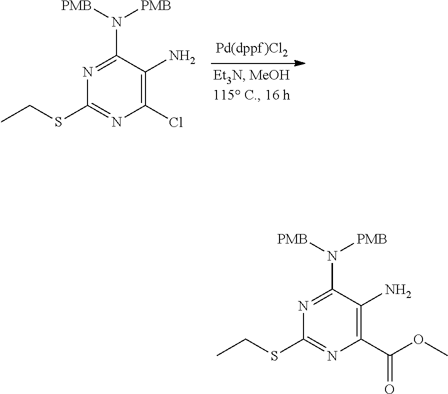

The starting material 6-chloro-2-(ethanethiol)-N,N-bis(4-methoxybenzyl)pyrimidine-4,5-diamine (12 g, 27.0 mmol, 1.0 eq) was dissolved in methanol (150 mL), and then triethylamine (13.5 g, 135.1 mmol, 5.0 eq) and Pd(dppf)Cl₂ (2.0 g, 2.7 mmol, 0.1 eq) were added. The reaction system was purged three times with carbon monoxide, charged with carbon monoxide and stirred at 115° C. for 20 h. After the reaction was completed, as detected by LC-MS, the reaction was concentrated to give a crude product, which was separated and purified by flash chromatography (PE:EA=8:1 to 6:1) to give the target compound (8.9 g, 70.6%). LC-MS: [M+H]⁺=469.11.

### Step 7: Preparation of methyl 6-(bis(4-methoxybenzyl)amino)-5-(bis(tert-butoxycarbonyl)amino)-2-(ethylthio)pyrimidine-4-carboxylate

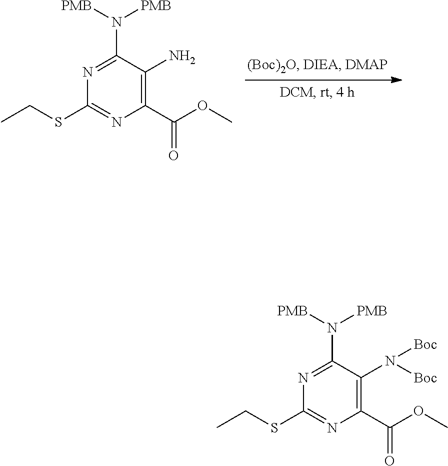

The starting material methyl 5-amino-6-(bis(4-methoxybenzyl)amino)-2-(ethylthio)pyrimidine-4-carboxylate (1.0 g, 2.1 mmol, 1.0 eq) was dissolved in dichloromethane (10 mL), and then DIEA (828.0 mg, 6.4 mmol, 3.0 eq), DMAP (261 mg, 2.1 mmol, 1.0 eq), and Boc anhydride (2.3 g, 10.5 mmol, 5.0 eq) were added. The mixture was stirred at 25° C. for 2 h. After the reaction was completed, as detected by LC-MS, water (10 mL) was added to the reaction mixture. The mixture was extracted with dichloromethane (10 mL×3). The organic phases were combined, washed with saturated brine (200 mL), dried over anhydrous sodium sulfate, filtered and concentrated to give a crude product, which was separated and purified by flash chromatography (PE:EA=20:1 to 10:1) to give the target compound (1.1 g, 77.1%). LC-MS: [M+H]⁺=669.41.

### Step 8: Preparation of tert-butyl (4-(bis(4-methoxybenzyl)amino)-2-(ethylthio)-6-(hydroxymethyl)pyrimidin-5-yl)carbamate

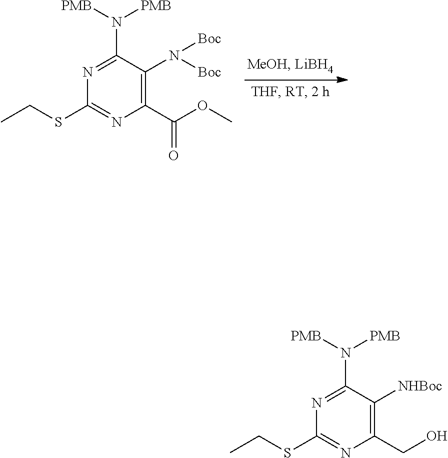

The starting material methyl 6-(bis(4-methoxybenzyl)amino)-5-(bis(tert-butoxycarbonyl)amino)-2-(ethylthio)pyrimidine-4-carboxylate (1.1 g, 1.60 mmol, 1.0 eq) was dissolved in tetrahydrofuran (10 mL), and then methanol (105 mg, 3.20 mmol, 2.0 eq), and lithium borohydride (71 mg, 3.20 mmol, 2.0 eq) were added. The reaction mixture was stirred at 25° C. for 2 h. After the reaction was completed, as detected by LC-MS, water (10 mL) was added to the reaction mixture. The mixture was extracted with ethyl acetate (10 mL×3). The organic phases were combined, washed with saturated brine (10 mL), dried over anhydrous sodium sulfate, filtered and concentrated to give the target compound (800 mg, 90%). LC-MS: [M+H]⁺=541.21.

### Step 9: Preparation of tert-butyl (4-(bis(4-methoxybenzyl)amino)-6-(bromomethyl)-2-(ethylthio)pyrimidin-5-yl)carbamate

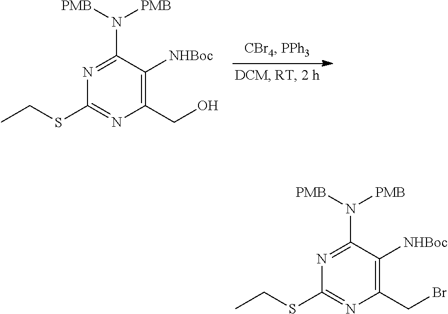

The starting material tert-butyl (4-(bis(4-methoxybenzyl)amino)-2-(ethylthio)-6-(hydroxymethyl)pyrimidin-5-yl)carbamate (800 mg, 0.70 mmol, 1.0 eq) was dissolved in dichloromethane (10 mL), and then triphenylphosphine (660 mg, 2.50 mmol, 1.7 eq) and tetrabromomethane (834 mg, 2.50 mmol, 1.7 eq) were added. The reaction was stirred at 25° C. for 2 h. After the reaction was completed, as detected by LC-MS, water (10 mL) was added to the reaction mixture, and then the mixture was extracted with dichloromethane (10 mL×2). The organic phases were combined, dried over anhydrous sodium sulfate, filtered and concentrated to give a crude product, which was separated and purified by flash chromatography (PE:EA=10:1 to 1:1) to give the target compound (440 mg, 49.3%). LC-MS: [M+H]⁺=605.21.

### Step 10: Preparation of tert-butyl (4-(bis(4-methoxybenzyl)amino)-2-(ethylthio)-6-((3-(pyrrolidin-1-ylmethyl)benzyl)amino)methyl)pyrimidin-5-yl)carbamate

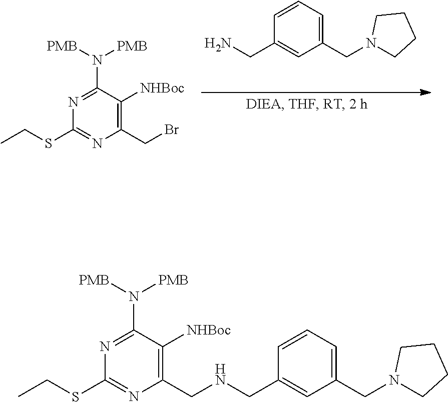

The starting material tert-butyl (4-(bis(4-methoxybenzyl)amino)-6-(bromomethyl)-2-(ethylthio)pyrimidin-5-yl)carbamate (440 mg, 0.70 mmol, 1.0 eq) was dissolved in tetrahydrofuran (5 mL), and then DIEA (283 mg, 2.20 mmol, 3.0 eq) and (3-(pyrrolidin-1-ylmethyl)phenyl)methylamine (167 mg, 0.90 mmol, 1.2 eq) were added. The reaction mixture was stirred at 25° C. for 2 h. After the reaction was completed, as detected by LC-MS, the reaction mixture was separated and purified by flash chromatography (DCM:MeOH=20:1 to 4:1) to give the target compound (250 mg, 48.1%). LC-MS: [M+H]⁺=713.51.

### Step 11: Preparation of 8-(bis(4-methoxybenzyl)amino)-6-(ethylthio)-3-(3-(pyrrolidin-1-ylmethyl)benzyl)-3,4-dihydropyrimido[5,4-d]pyrimidin-2(1H)-one

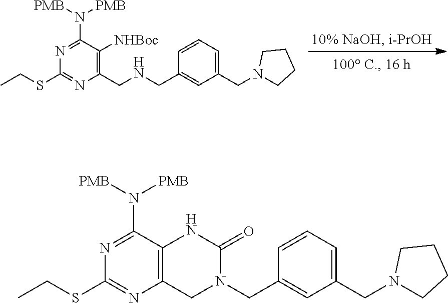

The starting material tert-butyl (4-(bis(4-methoxybenzyl)amino)-2-(ethylthio)-6-((3-(pyrrolidin-1-ylmethyl)benzyl)amino)methyl)pyrimidin-5-yl)carbamate (250 mg, 0.4 mmol, 1.0 eq) was dissolved in isopropanol (25 mL), and then 10% aqueous sodium hydroxide solution (5 mL) was added. The reaction mixture was stirred at 100° C. for 16 h. After the reaction was completed, as detected by LC-MS, the reaction mixture was concentrated. Water (25 mL) was added to the reaction mixture, and then the mixture was extracted with ethyl acetate (20 mL×3). The organic phases were combined, dried over anhydrous sodium sulfate, filtered and concentrated to give the target compound (198 mg, 88.3%). LC-MS: [M+H]⁺=639.51.

### Step 12: Preparation of 8-amino-6-(ethylthio)-3-(3-(pyrrolidin-1-ylmethyl)benzyl)-3,4-dihydropyrimido[5,4-d]pyrimidin-2(1H)-one hydrochloride

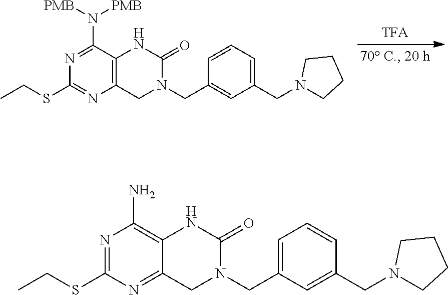

The starting material 8-(bis(4-methoxybenzyl)amino)-6-(ethylthio)-3-(3-(pyrrolidin-1-ylmethyl)benzyl)-3,4-dihydropyrimido[5,4-d]pyrimidin-2(1H)-one (195 mg, 0.30 mmol, 1.0 eq) was dissolved in trifluoroacetic acid (2 mL). The reaction was stirred at 70° C. for 16 h. After the reaction was completed, as detected by LC-MS, the reaction mixture was concentrated to give a crude product, which was separated and purified by Prep-HPLC (0.01% aqueous HCl solution, MeCN) to give the target compound (62 mg, 51.4%). LC-MS: [M+H]⁺=399.41; ¹H NMR (400 MHz, CD₃OD): δ 7.58 (s, 1H), 7.49 (m, 3H), 4.69 (s, 2H), 4.45 (s, 2H), 4.38 (s, 2H), 3.52-3.45 (m, 2H), 3.25 (q, J=7.2 Hz, 2H), 3.20-3.12 (m, 2H), 2.18 (m, 2H), 2.09-1.94 (m, 2H), 1.39 (t, J=7.2 Hz, 3H).

### Example 39: Preparation of 8-amino-6-butoxy-3-(3-(pyrrolidin-1-ylmethyl)phenethyl)-3,4-dihydropyrimido[5,4-d]pyrimidin-2(1H)-one

### Step 1: Preparation of 2-(3-(hydroxymethyl)phenyl)acetonitrile

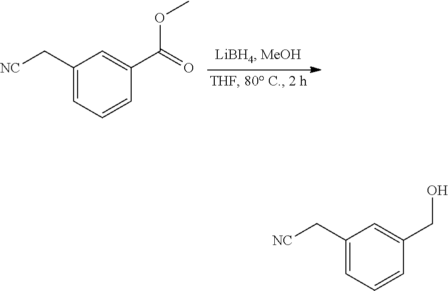

The starting material methyl 3-(cyanomethyl)benzoate (500 mg, 2.85 mmol, 1 eq.) was dissolved in tetrahydrofuran (5 mL). Methanol (183 mg, 5.71 mmol, 2 eq.) was added to the mixed solution described above, and then lithium borohydride (124 mg, 5.71 mmol, 2 eq.) was added slowly to the mixed solution described above. The reaction mixture was stirred at 80° C. for 3 h. After the reaction was completed, as detected by TLC, an aqueous hydrochloric acid solution was added to adjust the pH to 6. The mixture was extracted with ethyl acetate (20 mL×2). The organic phases were combined, washed with saturated brine (20 mL×2), dried over anhydrous sodium sulfate, filtered and concentrated to give the target compound (400 mg, 95.2%).

### Step 2: Preparation of 2-(3-(chloromethyl)phenyl)acetonitrile

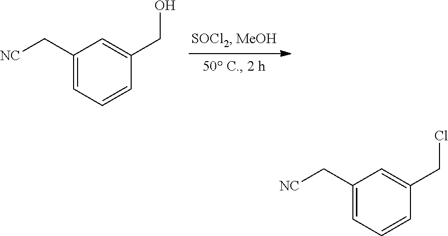 [SMILES: N#CCc1cccc(CO)c1 ; N#CCc1cccc(CCl)c1]

The starting material 2-(3-(hydroxymethyl)phenyl)acetonitrile (400 mg, 2.72 mmol, 1 eq.) was dissolved in thionyl chloride (4 mL, 33.65 mmol, 12.38 eq.). The reaction mixture was stirred at 50° C. for 2 h. After the reaction was completed, as detected by TLC, the reaction mixture was concentrated to give the target compound (350 mg, 77.4%). The crude product was used directly in the next step.

### Step 3: Preparation of 2-(3-(pyrrolidin-1-ylmethyl)phenyl)acetonitrile

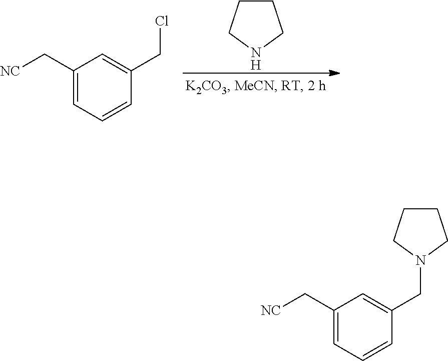 [SMILES: N#CCc1cccc(CCl)c1 ; C1CCNC1 ; N#CCc1cccc(CN2CCCC2)c1]

Tetrahydropyrrole (300 mg, 4.23 mmol, 2 eq.) and potassium carbonate (876 mg, 6.34 mmol, 3 eq.) were dissolved in acetonitrile (5 mL), and then a solution of 2-(3-(chloromethyl)phenyl)acetonitrile (350 mg, 2.11 mmol, 1 eq.) in acetonitrile (5 mL) was added dropwise. The reaction mixture was stirred at 25° C. for 2 h. After the reaction was completed, as detected by LC-MS, the reaction mixture was concentrated by rotary evaporation. The crude product was separated and purified by flash chromatography (DCM:MeOH=10:1) to give the target compound (350 mg, 82.7%). LC-MS: [M+H]⁺=201.21.

### Step 4: Preparation of 2-(3-(pyrrolidin-1-ylmethyl)phenyl)ethan-1-amine

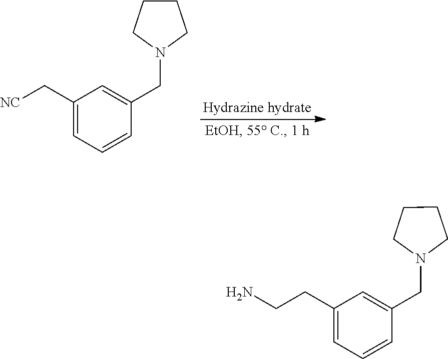 [SMILES: N#CCc1cccc(CN2CCCC2)c1 ; NCCc1cccc(CN2CCCC2)c1]

The starting material 2-(3-(pyrrolidin-1-ylmethyl)phenyl)acetonitrile (300 mg, 1.50 mmol, 1 eq.) was dissolved in ethanol (5 mL). Raney nickel (11 mg, 0.20 mmol, 0.1 eq.) and hydrazine hydrate (1 mL, 19.95 mmol, 10 eq.) were added sequentially. The mixture was stirred at 55° C. for 0.5 h. After the reaction was completed, as detected by LC-MS, the mixture was filtered, and the filtrate was concentrated to give the target compound (300 mg, 98%). LC-MS: [M+H]⁺=205.21.

### Step 5: Preparation of tert-butyl (4-(bis(4-methoxybenzyl)amino)-2-butoxy-6-((3-(pyrrolidin-1-ylmethyl)phenethyl)amino)methyl)pyrimidin-5-yl)carbamate

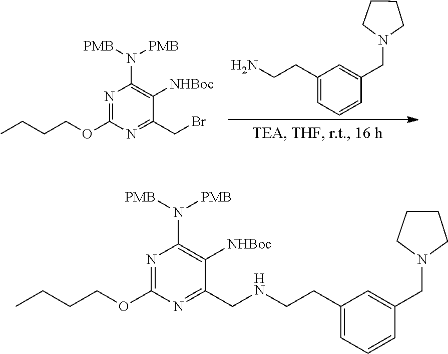

The starting material tert-butyl (4-(bis(4-methoxybenzyl)amino)-6-(bromomethyl)-2-butoxypyrimidin-5-yl)carbamate (300 mg, 0.49 mmol, 1 eq.) was dissolved in tetrahydrofuran (6 mL). 2-(3-(pyrrolidin-1-ylmethyl)phenyl)ethan-1-amine (139 mg, 0.68 mmol, 1.4 eq.) and triethylamine (148 mg, 1.46 mmol, 3 eq.) were added sequentially. The mixture was stirred at 25° C. for 16 h. After the reaction was completed, as detected by LC-MS, the mixture was concentrated by rotary evaporation and purified by silica gel column chromatography (DCM:MeOH=10:1) to give the target compound (220 mg, 61.1%). LC-MS: [M+H]⁺=739.31.

### Step 6: Preparation of 8-(bis(4-m ethoxybenzyl)amino)-6-butoxy-3-(3-(pyrrolidin-1-ylmethyl)phenethyl)-3,4-dihydropyrimido[5,4-d]pyrimidin-2(1H)-one

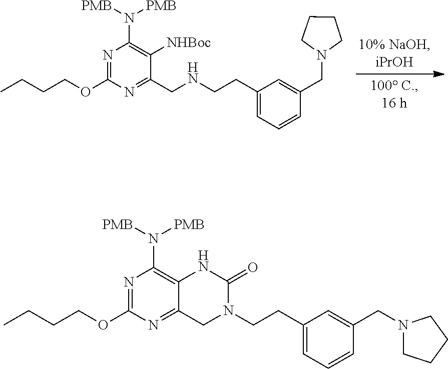

The starting material tert-butyl (4-(bis(4-methoxybenzyl)amino)-2-butoxy-6-((3-(pyrrolidin-1-ylmethyl)phenethyl)amino)methyl)pyrimidin-5-yl)carbamate (200 mg, 0.27 mmol, 1 eq.) was dissolved in 10% aqueous sodium hydroxide solution (1 mL) and isopropanol (5 mL). The reaction mixture was stirred at 100° C. for 16 h. After the reaction was completed, as detected by LC-MS, the reaction mixture was extracted with dichloromethane (20 mL×2). The organic phases were combined, washed with saturated brine (20 mL×2), dried over anhydrous sodium sulfate, filtered and concentrated to give the target compound (150 mg, 83.4%). LC-MS: [M+H]⁺=665.31.

### Step 7: Preparation of 8-amino-6-butoxy-3-(3-(pyrrolidin-1-ylmethyl)phenethyl)-3,4-dihydropyrimido[5,4-d]pyrimidin-2(1H)-one

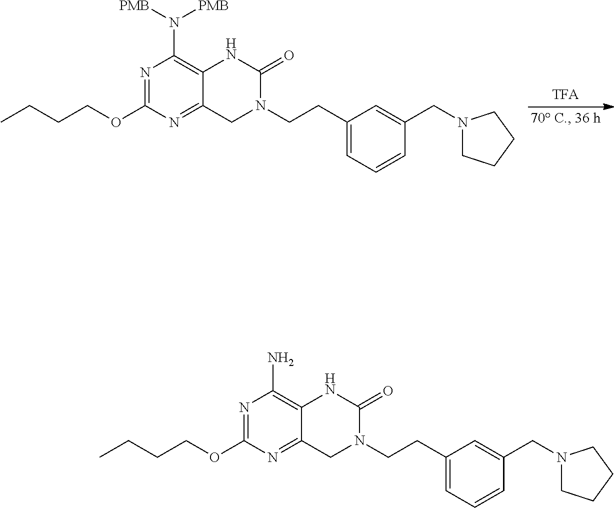

The starting material 8-(bis(4-methoxybenzyl)amino)-6-butoxy-3-(3-(pyrrolidin-1-ylmethyl)phenethyl)-3,4-dihydropyrimido[5,4-d]pyrimidin-2(1H)-one (200 mg, 0.30 mmol, 1 eq.) was dissolved in trifluoroacetic acid (5 mL). The reaction mixture was stirred at 70° C. for 32 h. After the reaction was completed, as detected by LC-MS, the reaction mixture was concentrated by rotary evaporation. The crude product was separated and purified by Prep-HPLC (0.01% aqueous HCl solution, MeCN) to give the target compound (17 mg, 13.3%). LC-MS: [M+H]⁺=425.41; ¹H NMR (400 MHz, DMSO-d₆): δ 11.02 (s, 1H), 8.83 (s, 1H), 8.04 (s, 1H), 7.51 (s, 1H), 7.46 (d, J=7.3 Hz, 1H), 7.40-7.28 (m, 2H), 4.35 (s, 2H), 4.30-4.14 (m, 4H), 3.57 (t, J=7.3 Hz, 2H), 3.28 (d, J=5.2 Hz, 2H), 3.08-2.93 (m, 2H), 2.86 (t, J=7.2 Hz, 2H), 1.98 (s, 2H), 1.95-1.81 (m, 2H), 1.72-1.59 (m, 2H), 1.47-1.32 (m, 2H), 0.92 (t, J=7.4 Hz, 3H).

### Example 40: Preparation of 2-(methylthio)-1-(4-(pyrrolidin-1-ylmethyl)benzyl)-1H-imidazo[4,5-c]quinoline-4-amine

### Step 1: Preparation of 4-(bis(4-methoxybenzyl)amino)-1-(4-(pyrrolidin-1-ylmethyl)benzyl)-1,3-dihydro-2H-imidazo[4,5-c]quinolin-2-thione

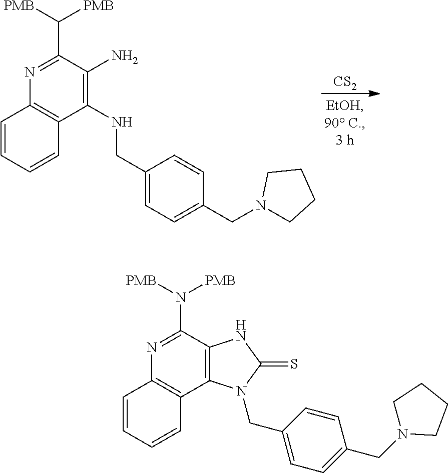

The starting material N²,N²-bis(4-methoxybenzyl)-N⁴-(4-(pyrrolidin-1 ylmethyl)benzyl)quinoline-2,3,4-triamine (600 mg, 1.02 mmol, 1 eq) was dissolved in ethanol (10 mL) and water (1 mL). Carbon disulfide (0.78 mL, 10.21 mmol, 10 eq) was added to the mixture described above. The reaction was stirred at 90° C. for 3 h. After the reaction was completed, as detected by LC-MS, the reaction mixture was concentrated by rotary evaporation. The crude product was separated and purified by flash chromatography (DCM:MeOH=10:1) to give the target product (400 mg, 62.2%). LC-MS: [M+H]⁺=630.1.

### Step 2: Preparation of N,N-bis(4-methoxybenzyl)-2-(methylthio)-1-(4-(pyrrolidin-1-ylmethyl)benzyl)-1H-imidazo[4,5-c]quinoline-4-amine

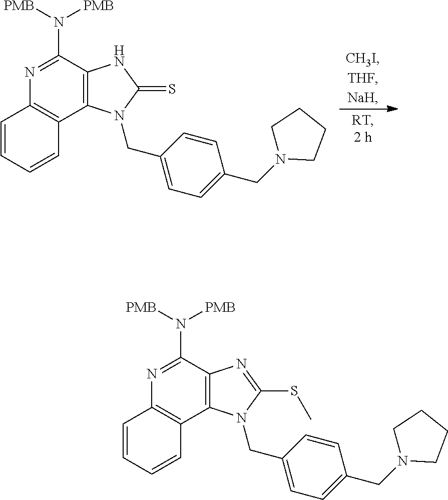

The starting material 4-(bis(4-methoxybenzyl)amino)-1-(4-(pyrrolidin-1-ylmethyl)benzyl)-1,3-dihydro-2H-imidazo[4,5-c]quinolin-2-thione (400 mg, 0.64 mmol, 1 eq) and sodium hydride (30.49 mg, 0.0013 mol, 2 eq) were dissolved in tetrahydrofuran (10 mL). The mixed solution was stirred at 25° C. for 10 min. Then iodomethane (72.12 mg, 0.51 mmol, 0.8 eq) was added. The reaction was stirred at 25° C. for 2 h. After the reaction was completed, as detected by LC-MS, the reaction mixture was concentrated by rotary evaporation. The crude product was separated and purified by flash chromatography (DCM:MeOH=10:1) to give the target product (270 mg, 66%) in the form of a yellow solid. LC-MS: [M+H]⁺=644.3.

### Step 3: Preparation of 2-(methylthio)-1-(4-(pyrrolidin-1-ylmethyl)benzyl)-1H-imidazo[4,5-c]quinoline-4-amine

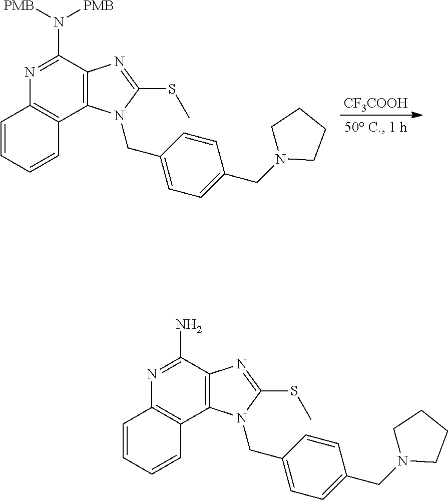

The starting material N,N-bis(4-methoxybenzyl)-2-(methylthio)-1-(4-(pyrrolidin-1-ylmethyl)benzyl)-1H-imidazo[4,5-c]quinolin-4-amine (40 mg, 0.062 mmol, 1 eq) was dissolved in trifluoroacetic acid (3 mL). The reaction mixture was stirred at 50° C. for 1 h. After the reaction was completed, as detected by LC-MS, the reaction mixture was concentrated. The crude product was separated and purified by Prep-HPLC (C18, 0.01% aqueous HCl solution, MeCN) to give the target compound (4 mg, 16%). LC-MS: [M+H]⁺=404.3. ¹H NMR (400 MHz, MeOD-d₄): δ 7.97 (d, J=8.2 Hz, 1H), 7.75 (d, J=9.0 Hz, 1H), 7.65 (t, J=7.9 Hz, 1H), 7.54 (d, J=7.6 Hz, 2H), 7.38 (t, J=7.5 Hz, 1H), 7.30 (d, J=7.9 Hz, 2H), 5.93 (s, 2H), 4.35 (s, 2H), 3.48-3.41 (m, 2H), 3.15 (d, J=12.7 Hz, 2H), 2.88 (s, 3H), 2.15 (t, J=8.9 Hz, 2H), 1.98 (t, J=7.0 Hz, 2H).

### Example 41: Preparation of 1-(3-((6-amino-2-butoxy-8-methoxy-9H-purin-9-yl)methyl)benzyl)pyrrolidin-2-one

### Step 1: Preparation of 1-(3-(bromomethyl)benzyl)pyrrolidin-2-one

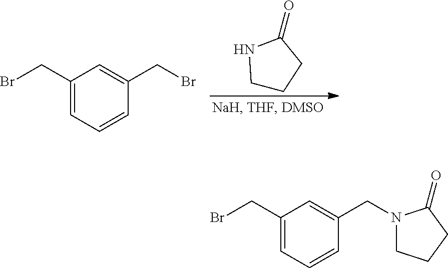 [SMILES: BrCc1cccc(CBr)c1 ; O=C1CCCN1 ; O=C1CCCN1Cc1cccc(CBr)c1]

Pyrrolidin-2-one (806 mg, 9.47 mmol, 726 μL, 0.5 eq) was dissolved in tetrahydrofuran (45 mL) and dimethylsulfoxide (6 mL). Sodium hydride (682 mg, 17.1 mmol, purity: 60%, 0.9 eq) was added at 0° C. under nitrogen atmosphere. After the mixture was stirred for 30 min, 1,3-bis(bromomethyl)benzene (5.00 g, 18.9 mmol, 1.0 eq) was added to the reaction mixture. The reaction mixture was stirred at 50° C. for 4 h. After the reaction was completed, as detected by TLC (PE:EA=3:1, 254 nm), the reaction system was quenched with water (20 mL) and extracted with ethyl acetate (20 mL×3). The organic phases were combined, washed with saturated brine (50 mL), dried over anhydrous sodium sulfate, filtered and concentrated to give a crude product, which was separated and purified by flash chromatography (PE:EA=100:0 to 33:66) to give the target compound (1.30 g, yield: 25.6%). ¹H NMR (400 MHz, DMSO-d₆): δ 7.35-7.34 (m, 2H), 7.29 (s, 1H), 7.17-7.15 (m, 1H), 4.70 (s, 2H), 4.36 (s, 2H), 3.24-3.21 (m, 2H), 2.32-2.28 (m, 2H), 1.94-1.91 (m, 2H).

### Step 2: Preparation of 2-chloro-9-(tetrahydro-2H-pyran-2-yl)-9H-purin-6-amine

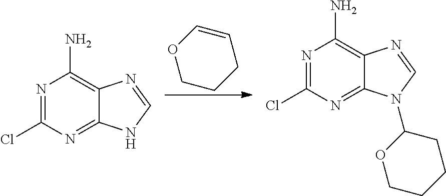

2-chloro-9H-purin-6-amine (23.0 g, 136 mmol, 1.0 eq) and p-toluenesulfonic acid monohydrate (2.30 g, 12.1 mmol, 0.09 eq) were dissolved in ethyl acetate (300 mL). 3,4-dihydro-2H-pyran (22.8 g, 271 mmol, 24.8 mL, 2.0 eq) was added at 50° C. After the addition, the mixture was stirred at 65° C. for 16 h. After the reaction was completed, as detected by TLC (PE:EA=1:1, 254 nm), the reaction system was filtered. The filtrate was adjusted to pH=8 with solid sodium carbonate. The organic phases were washed with saturated brine (200 mL), dried over anhydrous sodium sulfate, filtered and concentrated to give a crude product, which was separated and purified by flash chromatography (PE:EA=100:0 to 25:75) to give the target compound (20.0 g, yield: 52.3%). ¹H NMR (400 MHz, DMSO-d₆): δ 8.75 (d, J=7.6 Hz, 1H), 8.44 (s, 1H), 5.60-5.57 (m, 1H), 3.85-3.66 (m, 2H), 1.95-1.69 (m, 6H).

### Step 3: Preparation of 2-butoxy-9-(tetrahydro-2H-pyran-2-yl)-9H-purin-6-amine

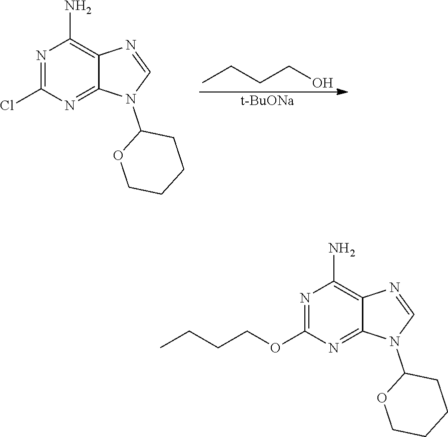

2-chloro-9-(tetrahydro-2H-pyran-2-yl)-9H-purin-6-amine (9.00 g, 35.5 mmol, 1.0 eq) was dissolved in n-butanol (90 mL) and slowly added dropwise to a solution of sodium tert-butoxide (34.1 g, 355 mmol, 10.0 eq) in n-butanol (60 mL). The reaction mixture was heated to 100° C. and stirred for 12 h. After the reaction was completed, as detected by TLC (DCM:MeOH=10:1, 254 nm) and LC-MS, the reaction mixture was cooled to room temperature. Water (300 mL) was poured into the reaction mixture and stirred for 15 min. The mixture was extracted with methyl tert-butyl ether (200 mL×3). The organic phases were combined, washed with saturated brine (100 mL), dried over anhydrous sodium sulfate, filtered and concentrated to give a crude product, which was separated and purified by flash chromatography (DCM:MeOH=100:0 to 91:9) to give the target compound (5.10 g, yield: 49.3%). LC-MS (ESI) [M+H]⁺=292.11.

### Step 4: Preparation of 8-bromo-2-butoxy-9-(tetrahydro-2H-pyran-2-yl)-9H-purin-6-amine

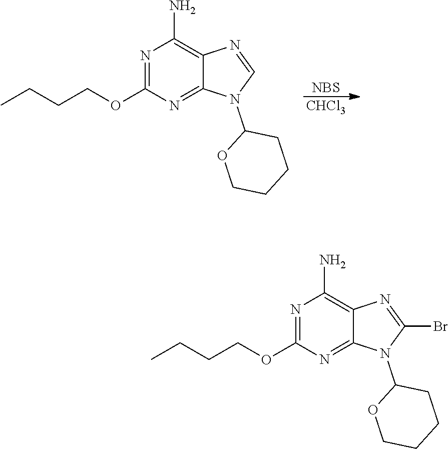

2-butoxy-9-(tetrahydro-2H-pyran-2-yl)-9H-purin-6-amine (2.00 g, 6.86 mmol, 1.0 eq) was dissolved in chloroform (30 mL). N-bromosuccinimide (3.67 g, 20.59 mmol, 3.0 eq) was added. The mixture was stirred at 25° C. for 16 h. After the reaction was completed, as detected by LC-MS, the reaction system was concentrated to give a crude product, which was separated and purified by flash chromatography (DCM:MeOH=100:0 to 94:6) to give the target compound (1.05 g, yield: 41.3%). LC-MS (ESI) [M+H]⁺=369.92.

### Step 5: Preparation of 2-butoxy-8-methoxy-9-(tetrahydro-2H-pyran-2-yl)-9H-purin-6-amine

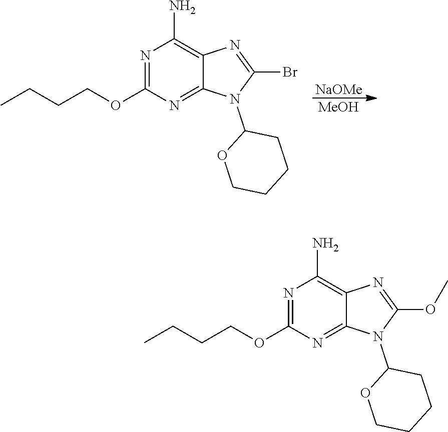 [SMILES: "MeOH" ; CCCCOc1nc(N)c2nc(Br)n(C3CCCCO3)c2n1 ; CCCCOc1nc(N)c2nc(OC)n(C3CCCCO3)c2n1 ; "NaOMe"]

8-bromo-2-butoxy-9-(tetrahydro-2H-pyran-2-yl)-9H-purin-6-amine (1.05 g, 2.84 mmol, 1.0 eq) was dissolved in methanol (5 mL), and then sodium methoxide (460 mg, 8.51 mmol, 3.0 eq) was added. The reaction mixture was stirred at 70° C. for 2 h. After the starting material was consumed completely, as detected by TLC (DCM:MeOH=20:1, 254 nm), water (10 mL) was added to the reaction mixture. The mixture was extracted with ethyl acetate (20 mL×2). The organic phases were combined, washed with saturated brine, dried over anhydrous sodium sulfate, filtered and concentrated to give a crude product of the target compound (500 mg, yield: 54.9%). The target compound was used directly in the next step without purification. ¹H NMR (400 MHz, DMSO-d₆): δ 6.82 (s, 1H), 5.39-5.33 (m, 1H), 4.21-3.96 (m, 6H), 3.60-3.57 (m, 1H), 2.71-2.67 (m, 1H), 2.05-1.86 (m, 1H), 1.72-1.62 (m, 4H), 1.44-1.38 (m, 3H), 0.94 (t, J=7.6 Hz, 3H).

### Step 6: Preparation of 2-butoxy-8-methoxy-9H-purin-6-amine

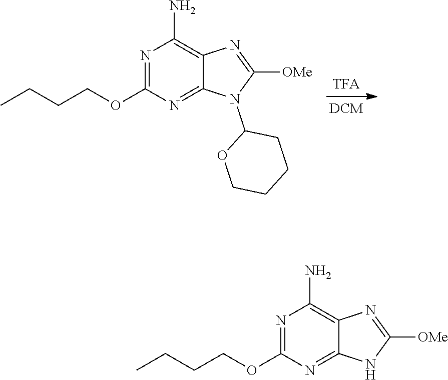 [SMILES: CCCCOc1nc(N)c2nc(OC)n(C3CCCCO3)c2n1 ; CCCCOc1nc(N)c2nc(OC)[nH]c2n1]

2-butoxy-8-methoxy-9-(tetrahydro-2H-pyran-2-yl)-9H-purin-6-amine (500 mg, 1.56 mmol, 1.0 eq) was dissolved in dichloromethane (5 mL), and then trifluoroacetic acid (1 mL) was added. The reaction mixture was stirred at 25° C. for 16 h. After the starting material was consumed completely, as detected by TLC (DCM:MeOH=20:1, 254 nm) and LC-MS, saturated sodium bicarbonate solution (10 mL) was added to the reaction mixture. The mixture was extracted with dichloromethane (10 mL×2). The organic phases were combined, washed with saturated brine, dried over anhydrous sodium sulfate, filtered and concentrated to give a crude product of the target compound (400 mg, yield: 73.2%). The target compound was used directly in the next step without purification. LC-MS (ESI) [M+H]⁺=238.08.

### Step 7: Preparation of 1-(3-((6-amino-2-butoxy-8-m ethoxy-9H-purin-9-yl)methyl)benzyl)pyrrolidin-2-one

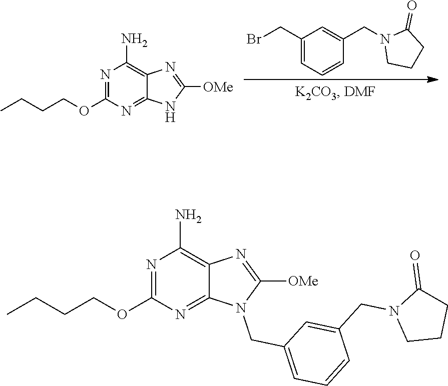 [SMILES: CCCCOc1nc(N)c2nc(OC)[nH]c2n1 ; O=C1CCCN1Cc1cccc(CBr)c1 ; CCCCOc1nc(N)c2nc(OC)n(Cc3cccc(CN4CCCC4=O)c3)c2n1]

2-butoxy-8-methoxy-9H-purin-6-amine (368 mg, 1.05 mmol, 1.0 eq) was dissolved in N,N-dimethylformamide (10 mL), and then 1-(3-(bromomethyl)benzyl)pyrrolidin-2-one (336 mg, 1.25 mmol, 1.2 eq) and potassium carbonate (443 mg, 3.14 mmol, 3.0 eq) were added. The reaction mixture was stirred at 25° C. for 16 h. After the starting material was consumed completely, as detected by LC-MS, water (10 mL) was added to the reaction mixture. The mixture was extracted with ethyl acetate (10 mL×3). The organic phases were combined, washed with saturated brine, dried over anhydrous sodium sulfate, filtered and concentrated to give a crude product, which was separated and purified by Prep-HPLC (0.01% aqueous formic acid, MeCN) to give the target compound (46.0 mg, yield: 10.3%). LC-MS (ESI) [M+H]⁺=425.2; ¹H NMR (400 MHz, CDCl₃): δ 7.31-7.26 (m, 1H), 7.22-7.18 (m, 3H), 5.07 (s, 2H), 4.49-4.46 (m, 2H), 4.43 (s, 2H), 4.15 (s, 3H), 3.26 (t, J=7.2 Hz, 2H), 2.46 (t, J=15.6 Hz, 2H), 2.02-1.99 (m, 2H), 1.84-1.80 (m, 2H), 1.48-1.44 (m, 2H), 0.97 (t, J=12.4 Hz, 3H).

### Example 42: Preparation of 6-amino-2-butoxy-9-(3-((2-oxopyrrolidin-1-yl)methyl)benzyl)-7,9-dihydro-8H-purin-8-one

### Step 1: Preparation of 6-amino-2-butoxy-9-(3-((2-oxopyrrolidin-1-yl)methyl)benzyl)-7,9-dihydro-8H-purin-8-one

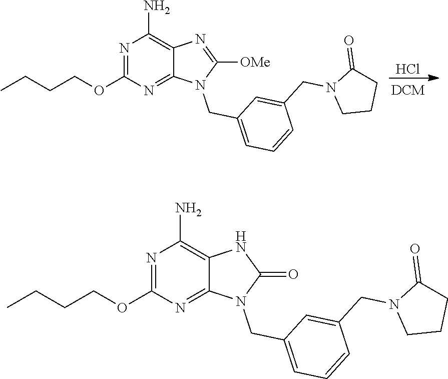

1-(3-((6-amino-2-butoxy-8-methoxy-9H-purin-9-yl)methyl)benzyl)pyrrolidin-2-one (46.0 mg, 0.11 mmol, 1.0 eq) was dissolved in dichloromethane (1.5 mL), and then a solution of hydrochloric acid in dioxane (1.5 mL, 4 M) was added. The reaction mixture was stirred at 25° C. for 2 h. After the starting material was consumed completely, as detected by LC-MS, the reaction mixture was directly concentrated to give a crude product, which was separated and purified by Prep-HPLC (0.01% aqueous formic acid, MeCN) to give the target compound (23.0 mg, yield: 51.7%). LC-MS (ESI) [M+H]⁺=411.1; ¹H NMR (400 MHz, DMSO-d₆): δ 10.56 (s, 1H), 7.30-7.27 (m, 1H), 7.20-7.18 (m, 1H), 7.12-7.09 (m, 2H), 4.86 (s, 2H), 4.32 (s, 2H), 4.21 (t, J=13.2 Hz, 2H), 3.16 (t, J=13.2 Hz, 2H), 2.25 (t, J=14.8 Hz, 2H), 1.90-1.87 (m, 2H), 1.64-1.58 (m, 2H), 1.36-1.30 (m, 2H), 0.90 (t, J=14.8 Hz, 3H).

### Example 43: Preparation of 6-amino-2-butoxy-9-((5-(pyrrolidin-1-ylmethyl)thien-2-yl)methyl)-7,9-dihydro-8H-purin-8-one

### Step 1: Preparation of 5-(bromomethyl)thiophene-2-carbaldehyde

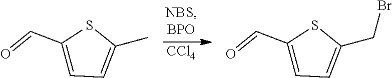

5-methylthiophene-2-carbaldehyde (2.00 g, 15.9 mmol, 1.00 eq) was dissolved in tetrachloromethane (20 mL), and then bromosuccinimide (3.10 g, 17.4 mmol, 1.10 eq) and benzoyl peroxide (115 mg, 475 μmol, 0.03 eq) were added. The reaction mixture was stirred at 80° C. for 20 h. After the reaction was completed, as detected by TLC (PE:EA=5:1, 254 nm), the reaction mixture was diluted with dichloromethane (100 mL) and filtered. The filtrate was washed with water (100 mL×2). The organic phases were dried over anhydrous sodium sulfate and concentrated to give a crude product, which was separated and purified by flash chromatography (PE:EA=100:1 to 80:20) to give the target compound (500 mg, yield: 15.4%).

### Step 2: Preparation of 5-(pyrrolidin-1-ylmethyl)thiophene-2-carbaldehyde

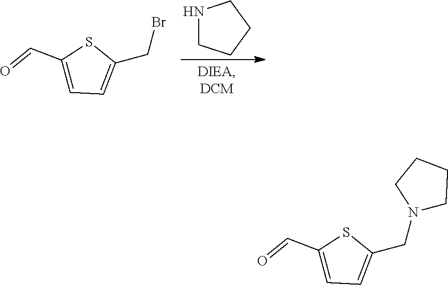

5-(bromomethyl)thiophene-2-carbaldehyde (3.5 g, 17.07 mmol, 1 eq), pyrrolidine (1.21 g, 17.1 mmol, 1.42 mL, 1.00 eq), and N,N-diisopropylethylamine (6.62 g, 51.2 mmol, 8.92 mL, 3.00 eq) were dissolved in dichloromethane (50 mL). The reaction mixture was stirred at 25° C. for 16 h. After the reaction was completed, as detected by TLC (DCM:MeOH=10:1, 254 nm), the reaction mixture was added dropwise to water (50 mL). The mixture was extracted with dichloromethane (50 mL×2). The organic phases were combined, dried over anhydrous sodium sulfate, filtered and concentrated to give a crude product, which was separated and purified by flash chromatography (DCM:MeOH=50:1 to 10:1) to give the target compound (1.40 g, yield: 42.0%). ¹H NMR (400 MHz, CDCl₃): δ 9.87 (s, 1H), 7.65 (d, J=3.6 Hz, 1H), 7.06 (d, J=3.6 Hz, 1H), 3.88 (s, 2H), 2.63-2.60 (m, 4H), 1.87-1.80 (m, 4H).

### Step 3: Preparation of (5-(pyrrolidin-1-ylmethyl)thien-2-yl)methanol

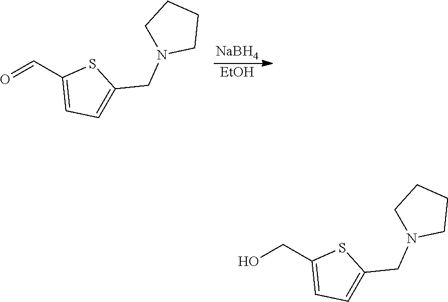

5-(pyrrolidin-1-ylmethyl)thiophene-2-carbaldehyde (1.00 g, 5.12 mmol, 1.00 eq) was dissolved in ethanol (20 mL). Sodium borohydride (290.60 mg, 7.68 mmol, 1.50 eq) was added in batches at 0° C. The reaction was stirred at 25° C. under nitrogen atmosphere for 16 h. After the reaction was completed, as detected by LC-MS, the reaction mixture was quenched with water (10 mL), and then extracted with dichloromethane (10 mL×2). The organic phases were combined, washed with saturated brine (10 mL), dried over sodium sulfate, filtered and concentrated to give a crude product, which was separated and purified by Prep-HPLC (0.01% aqueous ammonia, MeCN) to give the target compound (590 mg, yield: 58.4%). LC-MS (ESI) [M+H]⁺=198.04; ¹H NMR (400 MHz, CDCl₃): δ 6.76 (d, J=3.2 Hz, 1H), 6.70 (d, J=3.2 Hz, 1H), 4.68 (s, 2H), 3.70 (s, 2H), 2.48-2.43 (m, 4H), 1.72-1.71 (m, 4H).

### Step 4: Preparation of 1-((5-(chloromethyl)thien-2-yl)methyl)pyrrolidine

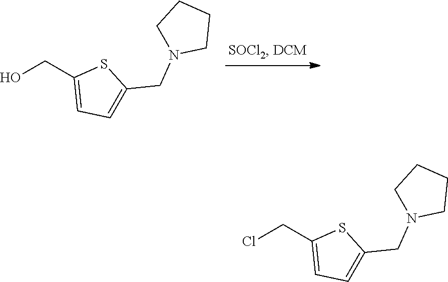 [SMILES: OCc1ccc(CN2CCCC2)s1 ; ClCc1ccc(CN2CCCC2)s1]

(5-(pyrrolidin-1-ylmethyl)thien-2-yl)methanol (500 mg, 2.53 mmol, 1.00 eq) was dissolved in dichloromethane (10 mL). Thionyl chloride (905 mg, 7.60 mmol, 552 μL, 3.00 eq) was added. The reaction mixture was stirred at 15° C. for 3 h. After the reaction was completed, as detected by LC-MS, the reaction mixture was concentrated to give a crude product (500 mg), which was used directly in the next step. LC-MS (ESI) [M+H]⁺=215.99.

### Step 5: Preparation of 2-butoxy-8-methoxy-9-((5-(pyrrolidin-1-ylmethyl)thien-2-yl)methyl)-9H-purin-6-amine

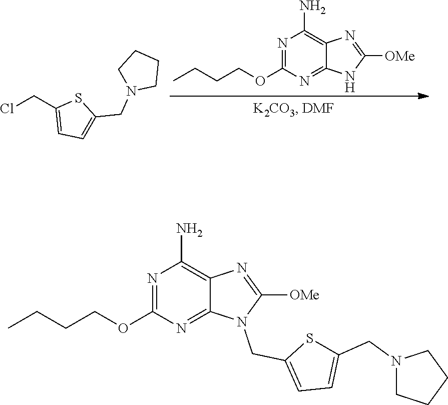

1-((5-(chloromethyl)thien-2-yl)methyl)pyrrolidine (500 mg, 2.32 mmol, 1.00 eq) and 2-butoxy-8-methoxy-9H-purin-6-amine (495 mg, 2.09 mmol, 0.90 eq) were dissolved in N,N-dimethylformamide (5 mL). Potassium carbonate (1.60 g, 11.6 mmol, 5.00 eq) was added. The reaction mixture was stirred at 15° C. for 16 h. After the reaction was completed, as detected by LC-MS, the reaction mixture was added dropwise to water (20 mL). The mixture was extracted with ethyl acetate (20 mL×3). The organic phases were combined, washed with saturated brine (30 mL×3), dried over sodium sulfate, filtered and concentrated to give a crude product, which was purified by prep-TLC (SiO₂, DCM:MeOH=5:1) to give a crude product, which was separated and purified by Prep-HPLC (0.01% trifluoroacetic acid in water, MeCN) to give the target compound (21.0 mg, yield: 2.18%). LC-MS (ESI) [M+H]⁺=417.2. ¹H NMR (400 MHz, CDCl₃): δ 7.09 (d, J=3.6 Hz, 1H), 7.02 (d, J=3.2 Hz, 1H), 5.21 (s, 2H), 4.47 (t, J=6.8 Hz, 2H), 4.34 (s, 2H), 4.17 (s, 3H), 3.67 (br s, 2H), 2.86 (br s, 2H), 2.11-2.06 (m, 4H), 1.85-1.79 (m, 2H), 1.51-1.44 (m, 2H), 0.98 (t, J=7.2 Hz, 3H).

### Step 6: Preparation of 6-amino-2-butoxy-9-((5-(pyrrolidin-1-ylmethyl)thien-2-yl)methyl)-7,9-dihydro-8H-purin-8-one

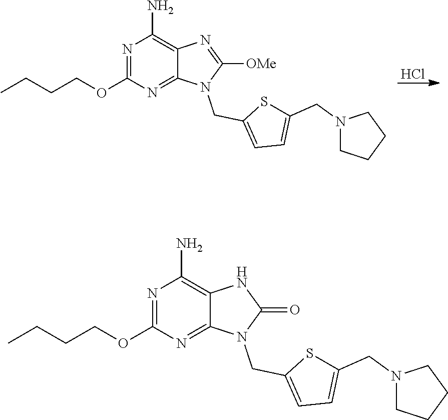 [SMILES: CCCCOc1nc(N)c2nc(OC)n(Cc3ccc(CN4CCCC4)s3)c2n1 ; Cl ; CCCCOc1nc(N)c2[nH]c(=O)n(Cc3ccc(CN4CCCC4)s3)c2n1]

2-butoxy-8-methoxy-9-((5-(pyrrolidin-1-ylmethyl)thien-2-yl)methyl)-9H-purin-6-amine (19.0 mg, 45.6 μmol, 1.00 eq) was dissolved in methanol (2 mL). A solution of hydrochloric acid in dioxane (4 M, 2 mL) was added. The reaction mixture was stirred at 15° C. for 16 h. After the reaction was completed, as detected by LC-MS, the reaction mixture was concentrated to give a crude product, which was separated and purified by Prep-HPLC (0.01% aqueous formic acid, MeCN) to give the target compound (3.88 mg, yield: 20.2%). LC-MS (ESI) [M+H]⁺=403.1; ¹H NMR (400 MHz, CD₃OD): δ 8.51 (s, 0.77 FA salt), 7.11 (d, J=3.6 Hz, 1H), 7.06 (d, J=3.6 Hz, 1H), 5.14 (s, 2H), 4.32-4.29 (m, 4H), 3.10-3.09 (m, 4H), 2.00-1.96 (m, 4H), 1.76-1.73 (m, 2H), 1.51-1.49 (m, 2H), 0.99 (t, J=3.6 Hz, 3H).

### Example 44: Preparation of 6-amino-2-butoxy-9-((5-((3-hydroxypyrrolidin-1-yl)methyl)thien-2-yl)methyl)-7,9-dihydro-8H-purin-8-one

### Step 1: Preparation of 5-((3-hydroxypyrrolidin-1-yl)methyl)thiophene-2-carbonitrile

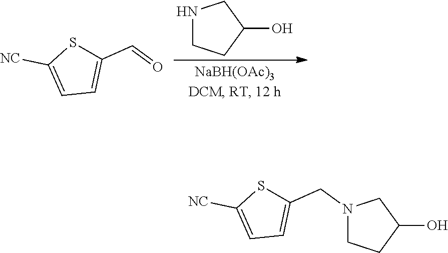

5-formylthiophene-2-carbonitrile (3 g, 21.87 mmol, 1 eq) was dissolved in dichloromethane (150 mL). Pyrrolidinol (2.1 g, 24.06 mmol, 1.1 eq) was added. The mixture was stirred at room temperature for 1 h. Sodium triacetoxyborohydride (9.27 g, 43.74 mmol, 2 eq) was added at 0° C. The reaction was stirred at 25° C. for 12 h. After the reaction was completed, as detected by LC-MS, the reaction mixture was quenched with saturated aqueous Na₂CO₃ solution (20 mL), extracted with dichloromethane (50 mL×2), and washed with saturated brine (50 mL×3). The organic phases were combined and concentrated by rotary evaporation to give the target product (5.9 g, yield: 88.9%). LC-MS (ESI) [M+H]⁺=209.1.

### Step 2: Preparation of 1-((5-(aminomethyl)thien-2-yl)methyl)pyrrolidin-3-ol

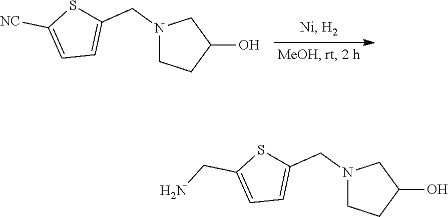 [SMILES: N#Cc1ccc(CN2CCC(O)C2)s1 ; NCc1ccc(CN2CCC(O)C2)s1]

5-((3-hydroxypyrrolidin-1-yl)methyl)thiophene-2-carbonitrile (5.9 g, 28.33 mmol, 1 eq) was dissolved in methanol (150 mL) and aqueous ammonia (15 mL). Raney nickel (3 g, 51.12 mmol, 1.8 eq) was added. The reaction was stirred under hydrogen atmosphere at 25° C. for 2 h. After the reaction was completed, as detected by LC-MS, the mixture was filtered. The filtrate was concentrated by rotary evaporation to give the target product (6 g, yield: 99.8%). LC-MS (ESI) [M+H]⁺=212.3.

### Step 3: Preparation of 1-((5-(((6-(bis(4-methoxybenzyl)amino)-2-butoxy-5-nitropyrimidin-4-yl)amino)methyl)thien-2-yl)methyl)pyrrolidin-3-ol

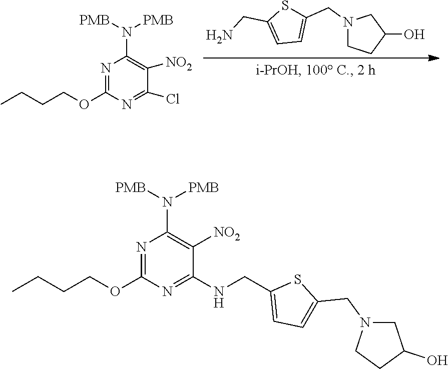

2-butoxy-6-chloro-N,N-bis(4-methoxybenzyl)-5-nitropyrimidin-4-amine (2 g, 4.11 mmol, 1 eq) was dissolved in isopropanol (20 mL). 1-((5-(aminomethyl)thien-2-yl)methyl)pyrrolidin-3-ol (1.31 g, 6.16 mmol, 1.5 eq) was added. The reaction was stirred at 100° C. for 2 h. After the reaction was completed, as detected by LC-MS, the reaction mixture was concentrated. The crude product was separated and purified by flash chromatography (silica gel, DCM:MeOH=50:1 to 10:1) to give the target compound (2 g, yield: 73.5%). LC-MS (ESI) [M+H]⁺=663.4.

### Step 4: Preparation of 1-((5-((5-amino-6-(bis(4-methoxybenzyl)amino)-2-butoxypyrimidin-4-yl)amino)methyl)thien-2-yl)methyl)pyrrolidin-3-ol

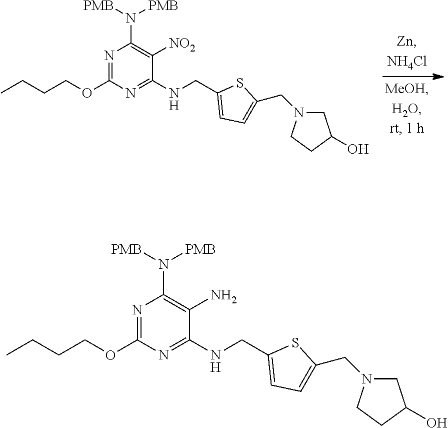

1-((5-(((6-(bis(4-methoxybenzyl)amino)-2-butoxy-5-nitropyrimidin-4-yl)amino)methyl)thien-2-yl)methyl)pyrrolidin-3-ol (1.9 g, 2.87 mmol, 1 eq) was dissolved in methanol (10 mL) and water (10 mL). Zinc powder (1.88 g, 28.7 mmol, 10 eq) and ammonium chloride (1.54 g, 28.7 mmol, 10 eq) were added to the reaction solution. The mixture was reacted at 25° C. for 1 h. After the reaction was completed, as detected by LC-MS, the mixture was filtered, washed with saturated sodium chloride (10 mL), and extracted with dichloromethane (20 mL×3). The organic phases were dried and concentrated to give the target product (1.5 g, yield: 82.7%). LC-MS (ESI) [M+H]⁺=633.5.

### Step 5: Preparation of 1-((5-((6-(bis(4-methoxybenzyl)amino)-2-butoxy-8-oxo-7,8-dihydro-9H-purin-9-yl)methyl)thien-2-yl)methyl)pyrrolidin-3-yl 1H-imidazole-1-carboxylate

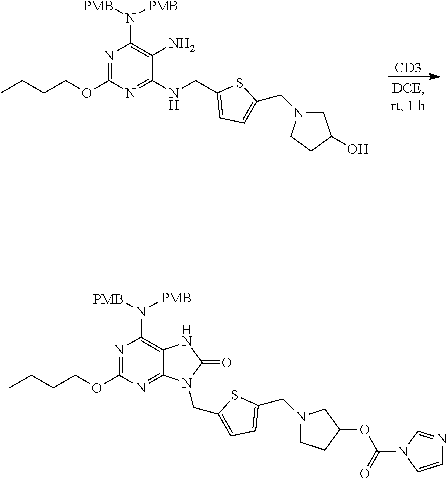

The compound 1-((5-((5-amino-6-(bis(4-methoxybenzyl)amino)-2-butoxypyrimidin-4-yl)amino)methyl)thien-2-yl)methyl)pyrrolidin-3-ol (200 mg, 0.32 mmol, 1 eq) was dissolved in 1,2-dichloroethane (10 mL). N,N-carbonyldiimidazole (512.47 mg, 3.16 mmol, 10 eq) was added. The mixture was reacted at 25° C. for 2 h. After the reaction was completed, as detected by LC-MS, the mixture was added with water (10 mL) for washing and extracted with dichloromethane (10 mL×3). The organic phases were dried over anhydrous sodium sulfate, filtered and concentrated to give a crude product of the target compound (210 mg, yield: 88.3%). LC-MS (ESI) [M+H]⁺=753.4. The crude product was used directly in the next step.

### Step 6: Preparation of 1-((5-((6-amino-2-butoxy-8-oxo-7,8-dihydro-9H-purin-9-yl)methyl)thien-2-yl)methyl)pyrrolidin-3-yl 1H-imidazole-1-carboxylate

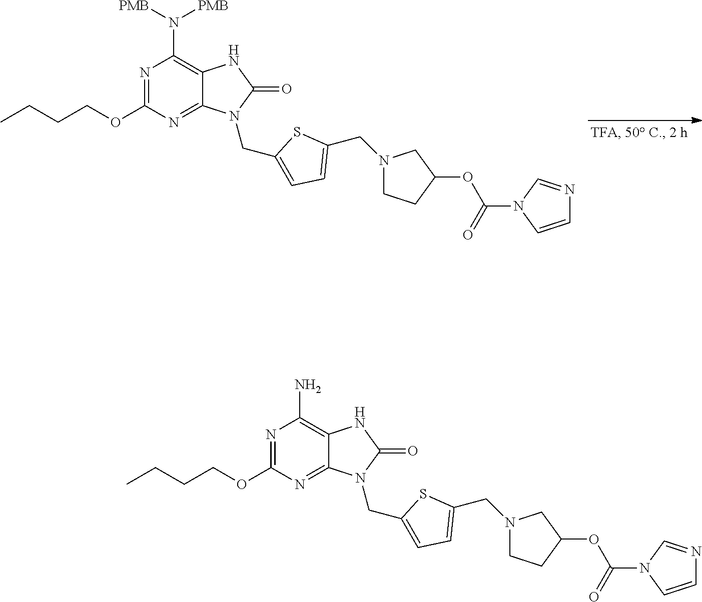

1-((5-((6-(bis(4-methoxybenzyl)amino)-2-butoxy-8-oxo-7,8-dihydro-9H-purin-9-yl)methyl)thien-2-yl)methyl)pyrrolidin-3-yl 1H-imidazole-1-carboxylate (210 mg, 0.28 mmol, 1 eq) was dissolved in trifluoroacetic acid (10 mL). The reaction mixture was stirred at 50° C. for 2 h. After the reaction was completed, as detected by LC-MS, the reaction mixture was concentrated by rotary evaporation to give the target compound (180 mg, yield: 25.18%). LC-MS (ESI) [M+H]⁺=513.2.

### Step 7: Preparation of 6-amino-2-butoxy-9-((5-((3-hydroxypyrrolidin-1-yl)methyl)thien-2-yl)methyl)-7,9-dihydro-8H-purin-8-one

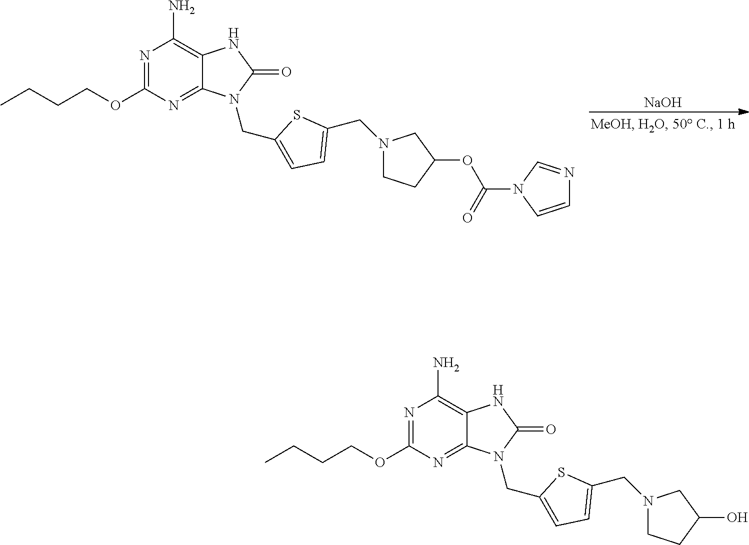 [SMILES: CCCCOc1nc(N)c2[nH]c(=O)n(Cc3ccc(CN4CCC(OC(=O)n5ccnc5)C4)s3)c2n1 ; "NaOH" ; CCCCOc1nc(N)c2[nH]c(=O)n(Cc3ccc(CN4CCC(O)C4)s3)c2n1]

1-((5-((6-amino-2-butoxy-8-oxo-7,8-dihydro-9H-purin-9-yl)methyl)thien-2-yl)methyl)pyrrolidin-3-yl 1H-imidazole-1-carboxylate (180 mg, 0.35 mmol, 1 eq) was dissolved in methanol (3 mL) and water (3 mL). Sodium hydroxide (70.23 mg, 1.76 mmol, 5 eq) was added. The reaction was stirred at 50° C. for 1 h. After the reaction was completed, as detected by LC-MS, the mixture was washed with water, extracted with dichloromethane (5 mL×3), dried over anhydrous sodium sulfate, and concentrated by rotary evaporation. The crude product was separated and purified by Prep-HPLC (C18, 0.01% aqueous ammonia, MeCN) to give the target compound (2.01 mg, yield: 1.4%). LC-MS (ESI) [M+H]⁺=419.2; ¹H NMR (400 MHz, DMSO-d₆) δ 6.86 (d, J=3.4 Hz, 1H), 6.74 (d, J=3.4 Hz, 1H), 6.46 (s, 2H), 4.94 (s, 2H), 4.66 (s, 1H), 4.17 (t, J=6.6 Hz, 2H), 3.64 (d, J=1.9 Hz, 2H), 2.69 (dd, J=9.6, 6.2 Hz, 2H), 2.48-2.37 (m, 2H), 2.34-2.24 (m, 1H), 1.94 (dd, J=13.1, 6.8 Hz, 1H), 1.64 (dd, J=14.5, 6.7 Hz, 2H), 1.51 (s, 1H), 1.39 (dd, J=14.9, 7.5 Hz, 2H), 0.92 (t, J=7.4 Hz, 3H).

### Example 45: Preparation of 6-amino-2-butoxy-9-((5-((4-glycylpiperazin-1-yl)methyl)thien-2-yl)methyl)-7,9-dihydro-8H-purin-8-one

### Step 1: Preparation of tert-butyl 4-((5-cyanothien-2-yl)methyl)piperazine-1-carboxylate

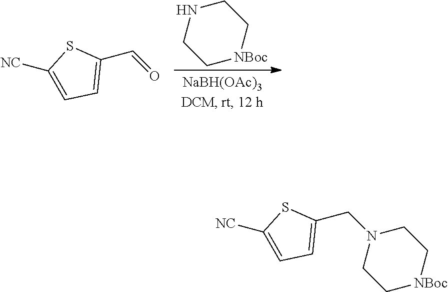

5-formylthiophene-2-carbonitrile (2.00 g, 14.58 mmol, 1 eq) was dissolved in dichloromethane (20 mL). Tert-butyl piperazine-1-carboxylate (2.99 g, 16.04 mmol, 1.1 eq) was added. The reaction system was stirred at room temperature for 1 h. Sodium triacetoxyborohydride (6.18 g, 29.16 mmol, 2 eq) was slowly added to the reaction mixture under an ice bath. After the addition, the ice bath was removed. The mixture was stirred at room temperature for 12 h. After the reaction was completed, as detected by LC-MS, saturated ammonium chloride solution (100 mL) was added to the reaction mixture and stirred well for half an hour. Water (30 mL) was added. The mixture was extracted with dichloromethane (100 mL×3). The organic phases were combined, dried over anhydrous sodium sulfate, filtered and concentrated to give a crude product (4.00 g, yield: 89.2%), which was used directly in the next step. LC-MS (ESI) [M+H]⁺=308.1; ¹H NMR (400 MHz, CDCl₃) δ 7.48 (d, J=3.8 Hz, 1H), 6.91 (d, J=3.8 Hz, 1H), 3.73 (s, 2H), 3.47-3.43 (m, 4H), 2.48-2.44 (m, 4H), 1.46 (s, 9H).

### Step 2: Preparation of tert-butyl 4-((5-(aminomethyl)thien-2-yl)methyl)piperazine-1-carboxylate

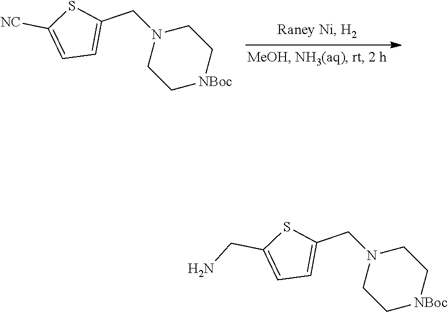 [SMILES: CC(C)(C)OC(=O)N1CCN(Cc2ccc(C#N)s2)CC1 ; CC(C)(C)OC(=O)N1CCN(Cc2ccc(CN)s2)CC1]

Raney nickel (3.72 g, 0.06 mol, 1 eq) was added to a mixed solution of tert-butyl 4-((5-cyanothien-2-yl)methyl)piperazine-1-carboxylate (3.90 g, 0.01 mol, 1 eq) in absolute methanol (40 mL) and aqueous ammonia (6 mL) at room temperature. The reaction system was stirred and reacted under hydrogen atmosphere for 12 h. After the reaction was completed, as detected by LC-MS, the mixture was filtered through celite and washed with methanol. The filtrate was concentrated to give a crude product (3.50 g, yield: 78.8%), which was used directly in the next step. LC-MS (ESI) [M+H]⁺=312.1.

### Step 3: Preparation of tert-butyl 4-((5-(((6-(bis(4-methoxybenzyl)amino)-2-butoxy-5-nitropyrimidin-4-yl)amino)methyl)thien-2-yl)methyl)piperazine-1-carboxylate

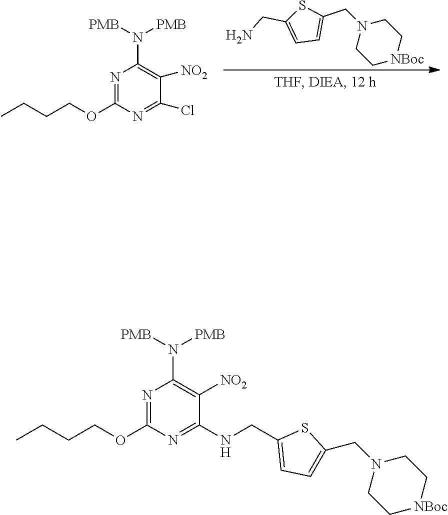

2-butoxy-6-chloro-N,N-bis((4-methoxyphenyl)methyl)-5-nitropyrimidin-4-amine (1400 mg, 2.88 mmol, 1 eq) was dissolved in isopropanol (15 mL). Tert-butyl 4-((5-(aminomethyl)thien-2-yl)methyl)piperazine-1-carboxylate (1343 mg, 4.31 mmol, 1.5 eq) was added. The reaction mixture was stirred at 100° C. for 20 h. After the reaction was completed, as detected by LC-MS, the isopropanol was removed by concentration to give a crude product, which was purified by a reversed-phase system (aqueous trifluoroacetic acid solution:ACN=100:0 to 20:80) to give the target compound (750 mg, yield: 34.2%). LC-MS (ESI) [M+H]⁺=762.3.

### Step 4: Preparation of tert-butyl 4-((5-((5-amino-6-(bis(4-methoxybenzyl)amino)-2-butoxypyrimidin-4-yl)amino)methyl)thien-2-yl)methyl)piperazine-1-carboxylate

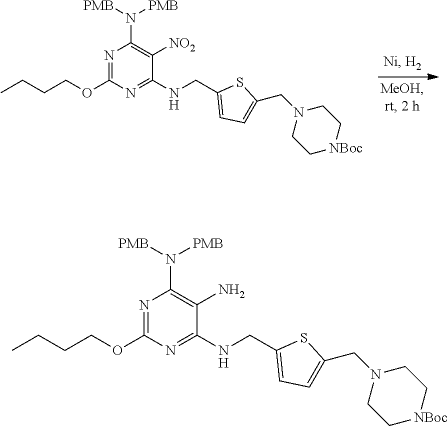

Tert-butyl 4-((5-((6-(bi s(4-m ethoxybenzyl)amino)-2-butoxy-5-nitropyrimidin-4-yl)amino)methyl)thien-2-yl)methyl)piperazine-1-carboxylate (720 mg, 0.94 mmol, 1 eq) was dissolved in methanol (10 mL) at room temperature. Raney nickel (277 mg, 4.72 mmol, 5 eq) was added. The reaction system was stirred under hydrogen atmosphere for 2 h. After the reaction was completed, as detected by LC-MS, the reaction system was filtered. The filtrate was concentrated to give a crude product, which was separated and purified through a reversed-phase column (C18, 0.5% aqueous formic acid, MeCN=30%-100%) to give the target compound (250 mg, yield: 36%). LC-MS (ESI) [M+H]⁺=732.3.

### Step 5: Preparation of tert-butyl 4-((5-((6-(bis(4-methoxybenzyl)amino)-2-butoxy-8-oxo-7,8-dihydro-9H-purin-9-yl)methyl)thien-2 yl)methyl)piperazine-1-carboxylate

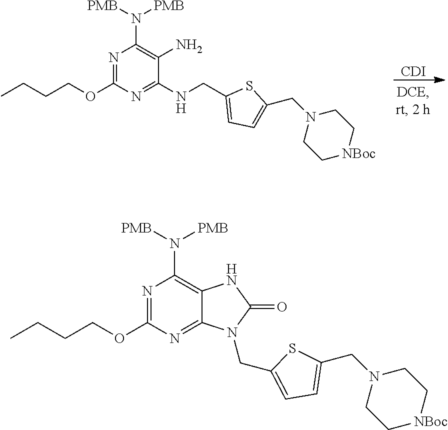

The compound tert-butyl 4-((5-((5-amino-6-(bis(4-methoxybenzyl)amino)-2-butoxypyrimidin-4-yl)amino)methyl)thien-2-yl)methyl)piperazine-1-carboxylate (200 mg, 0.27 mmol, 1 eq) was dissolved in 1,2-dichloroethane (5 mL). N,N-carbonyldiimidazole (443 mg, 2.73 mmol, 10 eq) was added. The mixture was reacted at 25° C. for 2 h. After the reaction was completed, as detected by LC-MS, water (10 mL) was added to the reaction system. The mixture was extracted with dichloromethane (10 mL×3). The organic phases were dried over anhydrous sodium sulfate, filtered to remove the solid, and concentrated by rotary evaporation to give a crude product (210 mg, yield: 50%). LC-MS (ESI) [M+H]⁺=758.5.

### Step 6: Preparation of 6-(bis(4-methoxybenzyl)amino)-2-butoxy-9-((5-(piperazin-1-ylmethyl)thien-2-yl)methyl)-7,9-dihydro-8H-purin-8-one

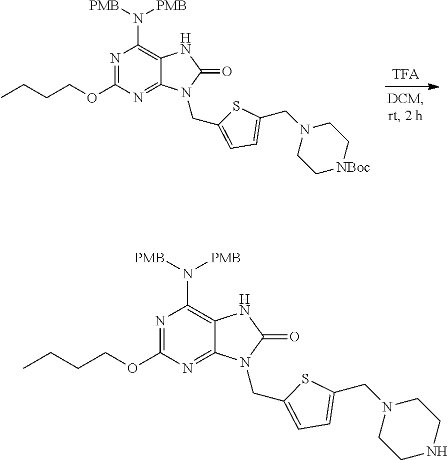

Tert-butyl 4-((5-((6-(bis(4-methoxybenzyl)amino)-2-butoxy-8-oxo-7,8-dihydro-9H-purin-9-yl)methyl)thien-2-yl)methyl)piperazine-1-carboxylate (200 mg, 0.26 mmol, 1 eq) was dissolved in dichloromethane (10 mL). A solution of trifluoroacetic acid (2 mL) diluted with dichloromethane (5 mL) was slowly added dropwise under stirring. The reaction mixture was stirred at 25° C. for 2 h. After the reaction was completed, as detected by LC-MS, the reaction system was quenched with saturated sodium bicarbonate solution, extracted with dichloromethane (5 mL×3), dried and concentrated to give a crude product (200 mg, yield: 70%), which was used directly in the next step. LC-MS (ESI) [M+H]⁺=658.5.

### Step 7: Preparation of tert-butyl (2-(4-((5-((6-(bis(4-methoxybenzyl)amino)-2-butoxy-8-oxo-7,8-dihydro-9H-purin-9-yl)methyl)thien-2-yl)methyl)piperazin-1-yl)-2-oxoethyl)carbamate

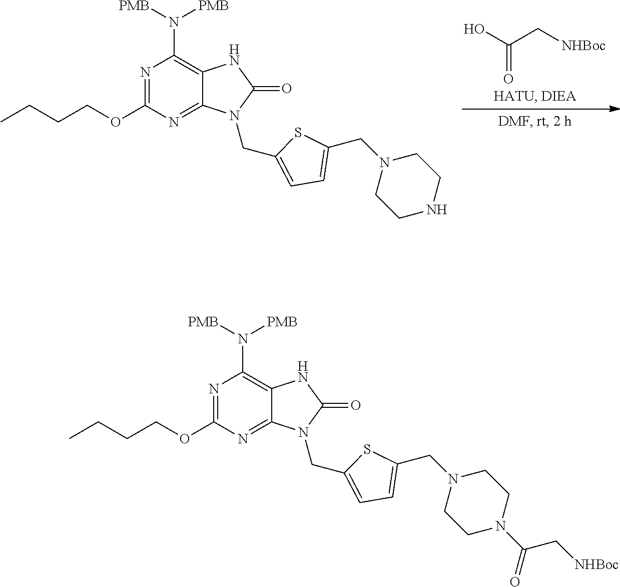

N-(tert-butoxycarbonyl)glycine (11 mg, 0.06 mmol, 1 eq) was dissolved in NA-dimethylformamide (5 mL). 2-(7-azabenzotriazol-1-yl)-N,N,N′,N′-tetramethyluronium hexafluorophosphate (28 mg, 0.07 mmol, 1.2 eq) was added. N,N-diisopropylethylamine (20 mg, 0.15 mmol, 2.5 eq) and a solution of 6-(bis(4-methoxybenzyl)amino)-2-butoxy-9-((5-(piperazin-1-ylmethyl)thien-2-yl)methyl)-7,9-dihydro-8H-purin-8-one (40 mg, 0.06 mmol, 1 eq) in N,N-dimethylformamide (2 mL) were added. The mixture was stirred at room temperature for 1.5 h. After the reaction was completed, as detected by LC-MS, the reaction mixture was quenched with water and extracted with ethyl acetate (10 mL×3). The organic phases were washed three times with saturated aqueous sodium chloride solution (5 mL). The organic phases were combined, dried over anhydrous sodium sulfate, and concentrated to give a crude product (60 mg, yield: 61.2%), which was used directly in the next step. LC-MS (ESI) [M+H]⁺=815.4.

### Step 8: Preparation of 6-amino-2-butoxy-9-((5-((4-glycylpiperazin-1-yl)methyl)thien-2-yl)methyl)-7,9-dihydro-8H-purin-8-one

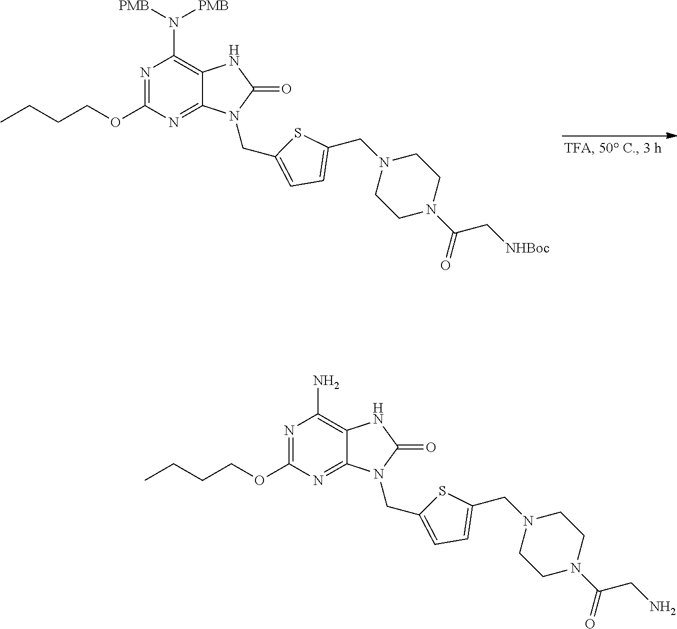

Tert-butyl (2-(4-((5-((6-(bis(4-methoxybenzyl)amino)-2-butoxy-8-oxo-7,8-dihydro-9H-purin-9-yl)methyl)thien-2-yl)methyl)piperazin-1-yl)-2-oxoethyl)carbamate (50 mg, 0.06 mmol, 1 eq) was dissolved in trifluoroacetic acid (10 mL). The mixture was stirred at 50° C. for 3 h. After the reaction was completed, as detected by LC-MS, the mixture was concentrated to remove the trifluoroacetic acid to give a crude product, which was separated and purified by Prep-HPLC (C18, 0.01% aqueous ammonia, MeCN) to give the target compound (11.72 mg, purity: 95.3%, yield: 40.3%). LC-MS (ESI) [M+H]⁺=475.3; ¹H NMR (400 MHz, DMSO-d₆) δ 6.89 (d, J=3.4 Hz, 1H), 6.78 (d, J=3.4 Hz, 1H), 6.46 (s, 2H), 4.95 (s, 2H), 4.17 (t, J=6.6 Hz, 2H), 3.61 (s, 2H), 3.44 (s, 2H), 3.36 (s, 2H), 3.32 (s, 4H), 2.37-2.29 (m, 4H), 1.69-1.62 (m, 2H), 1.39 (dd, J=15.0, 7.4 Hz, 2H), 0.92 (t, J=7.4 Hz, 3H).

### Examples 46-48

| | | | LC-MS |
|---|---|---|---|
| Ex- | | | (ESI) |
| ample | Chemical structure | ¹H NMR | [M + H]⁺ |
| 46 | 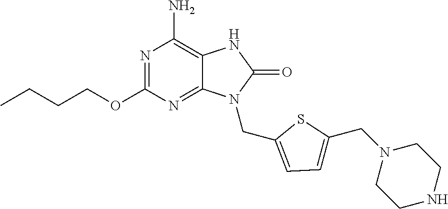 [SMILES: CCCCOc1nc(N)c2[nH]c(=O)n(Cc3ccc(CN4CCNCC4)s3)c2n1] | ¹H NMR (400 MHz, MeOD) δ 6.90 (d, J = 3.5 Hz, 1H), 6.70 (d, J = 3.4 Hz, 1H), 4.99 (s, 2H), 4.22 (t, J = 6.6 Hz, 2H), 3.56 (s, 2H), 2.76 (t, J = 4.9 Hz, 4H), 2.38 (s, 4H), 1.65 (dd, J = 14.8, 6.9 Hz, 2H), 1.40 (dd, J = 15.0, 7.5 Hz, 2H), 0.89 (t, J = 7.4 Hz, 3H) | 418.2 |
| 47 | 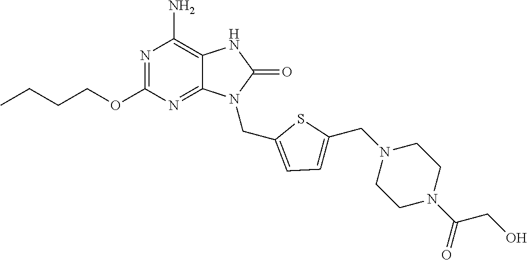 [SMILES: CCCCOc1nc(N)c2[nH]c(=O)n(Cc3ccc(CN4CCN(C(=O)CO)CC4)s3)c2n1] | ¹H NMR (400 MHz, MeOD) δ 7.25 (dd, J = 9.9, 3.5 Hz, 2H), 5.25 (s, 2H), 4.59 (s, 2H), 4.56 (t, J = 6.5 Hz, 2H), 4.28 (s, 2H), 3.50 (s, 2H), 3.34 (s, 4H), 3.15 (s, 2H), 1.89-1.80 (m, 2H), 1.55 (dd, J = 15.0, 7.5 Hz, 2H), 1.03 (t, J = 7.4 Hz, 3H). | 476.1 |
| 48 | 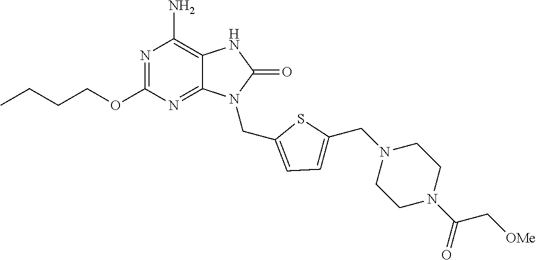 [SMILES: CCCCOc1nc(N)c2[nH]c(=O)n(Cc3ccc(CN4CCN(C(=O)COC)CC4)s3)c2n1] | ¹H NMR (400 MHz, MeOD) δ 7.13 (dd, J = 12.1, 3.5 Hz, 2H), 5.12 (s, 2H), 4.47 (s, 2H), 4.43 (t, J = 6.5 Hz, 2H), 4.08 (s, 2H), 3.50-3.32 (m, 2H), 3.29 (s, 3H), 3.22 (d, J = 1.6 Hz, 4H), 3.09 (d, J = 45.2 Hz, 2H), 1.79-1.67 (m, 2H), 1.43 (dd, J = 15.0, 7.4 Hz, 2H), 0.91 (t, J = 7.4 Hz, 3H). | 490.2 |

### Example 49: Preparation of 6-amino-9-((5-((3-aminopyrrolidin-1-yl)methyl)thien-2-yl)methyl)-2-butoxy-7,9-dihydro-8H-purin-8-one

The compound of Example 49 (16.31 mg, yield: 28%) was prepared by referring to the preparation method of Example 43. LC-MS (ESI) [M+H]⁺=418.2; ¹H NMR (400 MHz, DMSO-d₆) δ 6.86 (d, J=3.4 Hz, 1H), 6.74 (d, J=3.4 Hz, 1H), 6.46 (s, 2H), 4.94 (s, 2H), 4.17 (t, J=6.6 Hz, 2H), 3.65 (t, J=9.0 Hz, 2H), 2.67 (dd, J=9.1, 6.7 Hz, 2H), 2.49-2.39 (m, 2H), 2.10 (dd, J=9.0, 5.2 Hz, 1H), 1.96 (dd, J=13.0, 6.1 Hz, 1H), 1.69-1.60 (m, 2H), 1.45-1.27 (m, 3H), 0.92 (t, J=7.4 Hz, 3H).

### Example 50: Preparation of 8-amino-6-butoxy-3-(4-(pyrrolidin-1-ylcarbonyl)benzyl)-3,4-dihydropyrimido[5,4-d]pyrimidin-2(1H)-one

The compound of Example 50 (22.31 mg, yield: 24.3%) was prepared by referring to the preparation method of Example 34. LC-MS (ESI) [M+H]⁺=425.4; H NMR (400 MHz, MeOD) δ 7.54 (d, J=8.2 Hz, 2H), 7.45 (d, J=8.1 Hz, 2H), 4.69 (s, 2H), 4.47 (t, J=6.5 Hz, 2H), 4.38 (s, 2H), 3.59 (t, J=7.0 Hz, 2H), 3.45 (t, J=6.6 Hz, 2H), 1.98 (dd, J=13.8, 6.5 Hz, 2H), 1.91 (dd, J=12.8, 6.3 Hz, 2H), 1.82-1.72 (m, 2H), 1.47 (dd, J=15.0, 7.5 Hz, 2H), 0.97 (t, J=7.4 Hz, 3H).

### Example 51: Preparation of 8-amino-6-butoxy-3-(4-(piperidin-1-ylmethyl)benzyl)-3,4-dihydropyrimido[5,4-d]pyrimidin-2(1H)-one

The compound of Example 51 (0.019 g, yield: 29.8%) was prepared by referring to the preparation method of Example 34. LC-MS (ESI) [M+H]⁺=425.3; ¹H NMR (400 MHz, DMSO-d₆): δ 10.13 (s, 1H), 8.81 (s, 1H), 7.56 (d, J=8.0 Hz, 2H), 7.40 (d, J=8.0 Hz, 2H), 4.57 (s, 2H), 4.29-4.18 (m, 4H), 4.15 (s, 2H), 3.27-3.25 (m, 2H), 2.84-2.78 (m, 2H), 1.76-1.55 (m, 7H), 1.38-1.33 (m, 3H), 0.89 (t, J=7.2 Hz, 3H).

### Examples 52-61

The compounds of Examples 52-61 were prepared by referring to the preparation method of Example 34.

| | | | LC-MS |
|---|---|---|---|
| | | | (ESI) |
| Example | Chemical structure | ¹H NMR | [M + H]⁺ |
| 52 | 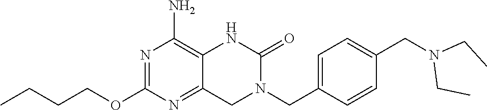 [SMILES: CCCCOc1nc(N)c2c(n1)CN(Cc1ccc(CN(CC)CC)cc1)C(=O)N2] | ¹H NMR (400 MHz, MeOD): δ 7.58 (d, J = 7.8 Hz, 2H), 7.51 (d, J = 7.8 Hz, 2H), 4.69 (s, 2H), 4.48 (t, J = 6.5 Hz, 2H), 4.44 (s, 2H), 4.35 (s, 2H), 3.23-3.18 (m, 4H), 1.82-1.70 (m, 2H), 1.48-1.46 (m, 2H), 1.35 (t, J = 7.2 Hz, 6H), 0.97 (t, J = 7.4 Hz, 3H). | 413.4 |
| 53 | 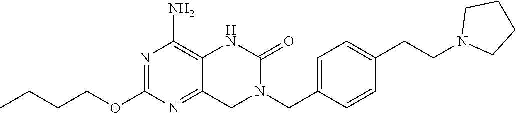 [SMILES: CCCCOc1nc(N)c2c(n1)CN(Cc1ccc(CCN3CCCC3)cc1)C(=O)N2] | ¹H NMR (400 MHz, DMSO_d6) δ 11.09 (s, 1H), 9.11 (s, 1H), 8.35 (s, 2H), 7.29 (s, 4H), 4.53 (s, 1H), 4.33-4.20 (m, 4H), 3.52 (m, 2H), 3.40-3.25 (m, 2H), 3.12- 2.91 (m, 4H), 2.09-1.94 (m, 2H), 1.90 (m, 2H), 1.73-1.56 (m, 2H), 1.46-1.26 (m, 2H), 0.91 (t, J = 7.4 Hz, 3H). | 425.2 |
| 54 | 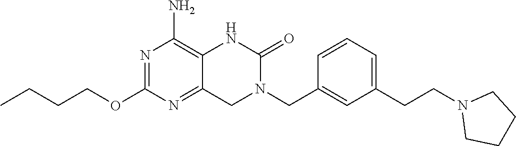 [SMILES: CCCCOc1nc(N)c2c(n1)CN(Cc1cccc(CCN3CCCC3)c1)C(=O)N2] | ¹H NMR (400 MHz, MeOD): δ 7.41-7.25 (m, 4H), 4.64 (s, 2H), 4.48-4.47 (m, 2H), 4.40 (s, 2H), 3.71-3.63 (m, 2H), 3.49- 3.42 (m, 2H), 3.16-3.05 (m, 4H), 2.21- 2.12 (m, 2H), 2.06-1.98 (m, 2H), 1.81- 1.72 (m, 2H), 1.50-1.44 (m, 2H), 0.97 (t, J = 7.1 Hz, 3H). | 425.3 |
| 55 | 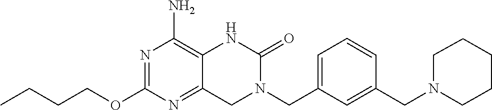 [SMILES: CCCCOc1nc(N)c2c(n1)CN(Cc1cccc(CN3CCCCC3)c1)C(=O)N2] | ¹H NMR (400 MHz, DMSO-d₆) δ 10.74 (s, 1H), 9.11 (s, 1H), 8.21 (s, 1H), 7.60 (d, J = 5.9 Hz, 2H), 7.51 (t, J = 7.8 Hz, 1H), 7.45 (d, J = 7.6 Hz, 1H), 4.65 (s, 2H), 4.37 (s, 2H), 4.31 (dd, J = 7.5, 6.2 Hz, 4H), 3.31 (d, J = 11.5 Hz, 2H), 2.88 (t, J = 10.5 Hz, 2H), 1.84 (t, J = 6.1 Hz, | 425.4 |
| | | 4H), 1.77-1.66 (m, 3H), 1.45-1.43 (m, | |
| | | 3H), 0.97 (t, J = 7.4 Hz, 3H). | |
| 56 | 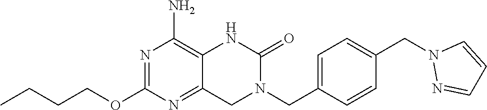 [SMILES: CCCCOc1nc(N)c2c(n1)CN(Cc1ccc(Cn3cccn3)cc1)C(=O)N2] | ¹H NMR (400 MHz, DMSO-d6) δ 8.78 (d, J = 33.2 Hz, 1H), 7.81 (d, J = 2.0 Hz, 1H), 7.45 (d, J = 1.5 Hz, 1H), 7.28 (d, J = 8.0 Hz, 2H), 7.20 (d, J = 8.0 Hz, 2H), 6.26 (t, J = 2.0 Hz, 1H), 5.32 (s, 2H), 4.50 (s, 2H), 4.15 (d, J = 6.9 Hz, 4H), 1.61 (s, 2H), 1.34 (dt, J = 15.0, 7.6 Hz, | 408.2 |
| | | 2H), 0.89 (t, J = 7.4 Hz, 3H). | |
| 57 | 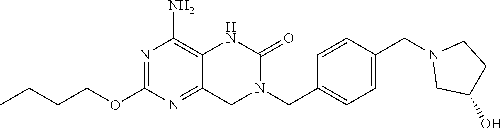 [SMILES: CCCCOc1nc(N)c2c(n1)CN(Cc1ccc(CN3CC[C@H](O)C3)cc1)C(=O)N2] | ¹H NMR (400 MHz, MeOD) δ 7.37- 7.29 (m, 4H), 4.60 (s, 2H), 4.36-4.28 (m, 1H), 4.23-4.11 (m, 4H), 3.70-3.56 (m, 2H), 2.87-2.66 (m, 2H), 2.59-2.41 (m, 2H), 2.18-2.04 (m, 1H), 1.73-1.61 (m, 3H), 1.48-1.38 (m, 2H), 0.94 (t, J = 7.4 Hz, 3H). | 427.2 |
| 58 | 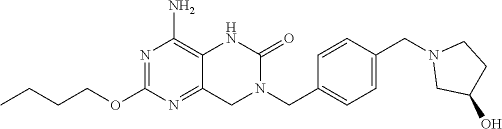 [SMILES: CCCCOc1nc(N)c2c(n1)CN(Cc1ccc(CN3CC[C@@H](O)C3)cc1)C(=O)N2] | ¹H NMR (400 MHz, MeOD) δ 7.55 (d, J = 3.9 Hz, 2H), 7.49 (d, J = 7.7 Hz, 2H), 4.68 (s, 2H), 4.56 (s, 1H), 4.45 (dd, J = 13.3, 6.8 Hz, 4H), 4.37 (d, J = 14.2 Hz, 2H), 3.71-3.42 (m, 2H), 3.18 (d, J = 12.6 Hz, 2H), 2.13 (dd, J = 119.9, 68.4 Hz, 2H), 1.82-1.66 (m, 2H), 1.46 (dd, J = 15.0, 7.6 Hz, 2H), 0.97 (t, J = 7.4 Hz, | 427.2 |
| | | 3H). | |
| 59 | 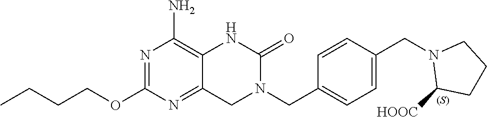 | ¹H NMR (400 MHz, MeOD) δ 7.53 (d, J = 8.0 Hz, 2H), 7.45 (d, J = 7.9 Hz, 2H), 4.64 (d, J = 4.5 Hz, 2H), 4.49 (d, J = 12.9 Hz, 1H), 4.19 (dd, J = 11.7, 5.1 Hz, 5H), 3.87 (t, J = 7.8 Hz, 1H), 3.47 (t, J = 9.4 Hz, 1H), 3.18 (d, J = 9.7 Hz, 1H), 2.51- 2.36 (m, 1H), 2.17-2.03 (m, 2H), 2.01- 1.85 (m, 1H), 1.74-1.60 (m, 2H), 1.44 | 455.2 |
| | | (dd, J = 15.0, 7.5 Hz, 2H), 0.94 (t, J = 7.4 | |
| | | Hz, 3H). | |
| 60 | 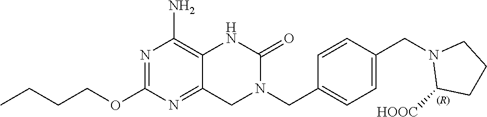 | ¹H NMR (400 MHz, MeOD) δ 7.52 (d, J = 8.0 Hz, 2H), 7.44 (d, J = 8.0 Hz, 2H), 4.64 (s, 2H), 4.47 (d, J = 13.6 Hz, 1H), 4.20 (s, 2H), 4.17 (d, J = 6.5 Hz, 2H), 3.82 (s, 1H), 3.42 (s, 2H), 3.13-3.11 (m, 1H), 2.42-2.41 (m, 1H), 2.08-2.07 (m, 2H), 1.91 (d, J = 16.0 Hz, 1H), 1.72- 1.63 (m, 2H), 1.44 (dd, J = 14.9, 7.6 Hz, | 455.2 |
| | | 2H), 0.94 (t, J = 7.4 Hz, 3H). | |
| 61 | 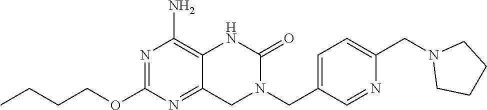 [SMILES: CCCCOc1nc(N)c2c(n1)CN(Cc1ccc(CN3CCCC3)nc1)C(=O)N2] | ¹H NMR (400 MHz, MeOD) δ 8.51 (d, J = 1.7 Hz, 1H), 7.85-7.79 (m, 1H), 7.51 (d, J = 8.0 Hz, 1H), 4.65 (s, 2H), 4.25 (s, 2H), 4.19 (t, J = 6.6 Hz, 2H), 3.80 (s, 2H), 2.62 (s, 4H), 1.84-1.80 (m, 4H), 1.70-1.64 (m, 2H), 1.48-1.41 (m, 2H), 0.95 (t, J = 7.4 Hz, 3H). | 412.2 |

### Example 62: Preparation of 8-amino-6-butoxy-3-((5-(pyrrolidin-1-ylmethyl) pyridin-2-yl)methyl)-3,4-dihydropyrimido[5,4-d]pyrimidin-2(1H)-one

The compound of Example 62 (6.72 mg, yield: 22.2%) was prepared by referring to the preparation method of Example 34. LC-MS (ESI) [M+H]⁺=412.2; ¹H NMR (400 MHz, DMSO-d₆) δ 8.56 (s, 1H), 8.43 (s, 1H), 7.69 (d, J=8.0 Hz, 1H), 7.28 (d, J=7.8 Hz, 1H), 6.85 (s, 2H), 4.59 (s, 2H), 4.29 (s, 2H), 4.06 (t, J=6.6 Hz, 2H), 3.58 (s, 2H), 2.42 (s, 4H), 1.69 (s, 4H), 1.63-1.55 (m, 2H), 1.35 (dd, J=14.8, 7.5 Hz, 2H), 0.89 (t, J=7.4 Hz, 3H).

### Example 63: Preparation of 8-amino-6-butoxy-3-((6-(4-methylpiperazin-1-yl) pyridin-3-yl)methyl)-3,4-dihydropyrimido[5,4-d]pyrimidin-2(1H)-one

The compound of Example 63 (6.43 mg, yield: 11.6%) was prepared by referring to the preparation method of Example 34. LC-MS (ESI) [M+H]⁺=427.3; ¹H NMR (400 MHz, DMSO-d₆) δ 8.54 (s, 1H), 8.09 (d, J=2.2 Hz, 1H), 7.54-7.47 (m, 1H), 6.81 (d, J=8.8 Hz, 3H), 4.37 (s, 2H), 4.10 (s, 2H), 4.05 (t, J=6.6 Hz, 2H), 3.49-3.42 (m, 4H), 2.40-2.34 (m, 4H), 2.20 (s, 3H), 1.62-1.54 (m, 2H), 1.39-1.30 (m, 2H), 0.88 (t, J=7.4 Hz, 3H).

### Example 64: Preparation of 8-amino-6-butoxy-3-((6-(2-(dimethylamino) ethoxy)pyridin-3-yl)methyl)-3,4-dihydropyrimido[5,4-d]pyrimidin-2(1H)-one

The compound of Example 64 (8.8 mg, yield: 14.1%) was prepared by referring to the preparation method of Example 34. LC-MS (ESI) [M+H]⁺=416.3; ¹H NMR (400 MHz, MeOD) S 8.13 (d, J=2.2 Hz, 1H), 7.70 (dd, J=8.6, 2.4 Hz, 1H), 6.81 (d, J=8.5 Hz, 1H), 4.54 (s, 2H), 4.41 (t, J=5.6 Hz, 2H), 4.22-4.14 (m, 4H), 2.76 (t, J=5.6 Hz, 2H), 2.33 (s, 6H), 1.72-1.61 (m, 2H), 1.48-1.37 (m, 2H), 0.94 (t, J=7.4 Hz, 3H).

### Examples 65-94

The compounds of Examples 65-94 were prepared by referring to the preparation method of Example 34

| | | | LC-MS |
|---|---|---|---|
| | | | (ESI) |
| Example | Chemical structure | ¹H NMR | [M + H]⁺ |
| 65 | 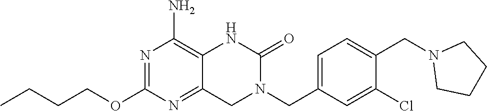 [SMILES: CCCCOc1nc(N)c2c(n1)CN(Cc1ccc(CN3CCCC3)c(Cl)c1)C(=O)N2] | ¹H NMR (400 MHz, MeOD): δ 7.71 (d, J = 7.6 Hz, 1H), 7.60 (s, 1H), 7.47 (d, J = 7.6 Hz, 1H), 4.67 (s, 2H), 4.57 (s, 2H), 4.50-4.43 (m, 4H), 3.62-3.52 (m, 2H), 3.31 (s, 2H), 2.26-2.15 (m, 2H), 2.10- | 445.1 |
| | | 1.99 (m, 2H), 1.82-1.72 (m, | |
| | | 2H), 1.52-1.43 (m, 2H), 0.98 | |
| | | (t, J = 7.4 Hz, 3H). | |
| 66 | 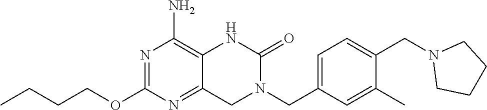 [SMILES: CCCCOc1nc(N)c2c(n1)CN(Cc1ccc(CN3CCCC3)c(C)c1)C(=O)N2] | ¹H NMR (400 MHz, DMSO- d₆): δ 10.55 (s, 1H), 8.95 (s, 1H), 7.94 (s, 2H), 7.59 (d, J = 7.6 Hz, 1H), 7.20 (d, J = 8.5 Hz, 2H), 4.53 (s, 2H), 4.34 (d, J = 5.8 Hz, 2H), 4.26-4.17 (m, 4H), 3.39 (m, 2H), 3.09 | 425.1 |
| | | (m, 2H), 2.42 (s, 3H), 2.01 | |
| | | (m, 2H), 1.91 (m, 2H), 1.70- | |
| | | 1.56 (m, 2H), 1.48-1.26 (m, | |
| | | 2H), 0.90 (t, J = 7.4 Hz, 3H). | |
| 67 | 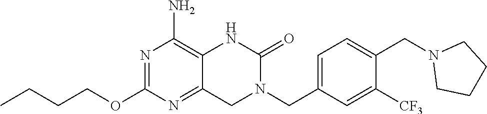 [SMILES: CCCCOc1nc(N)c2c(n1)CN(Cc1ccc(CN3CCCC3)c(C(F)(F)F)c1)C(=O)N2] | ¹H NMR (400 MHz, MeOD) δ 7.92-7.78 (m, 3H), 4.74 (s, 2H), 4.60 (s, 2H), 4.46-4.37 (m, 4H), 3.61 (s, 2H), 3.29- 3.24 (m, 2H), 2.21 (s, 2H), 2.05 (s, 2H), 1.79-1.72 (m, 2H), 1.51-1.42 (m, 2H), 0.97 (t, J = 7.4 Hz, 3H). | 479.3 |
| 68 | 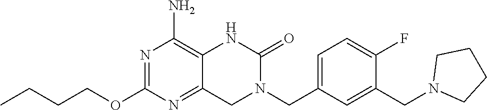 [SMILES: CCCCOc1nc(N)c2c(n1)CN(Cc1ccc(F)c(CN3CCCC3)c1)C(=O)N2] | ¹H NMR (400 MHz, MeOD) δ 7.41 (t, J = 7.7 Hz, 1H), 7.13 (dd, J = 20.7, 9.3 Hz, 2H), 4.61 (s, 2H), 4.19 (dd, J = 11.8, 5.2 Hz, 4H), 3.70 (s, 2H), 2.59 (s, 4H), 1.80 (s, 4H), 1.72-1.62 (m, 2H), | 429.2 |
| | | 1.44 (dd, J = 15.1, 7.4 Hz, | |
| | | 2H), 0.94 (t, J = 7.4 Hz, 3H). | |
| 69 | 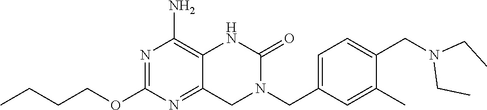 [SMILES: CCCCOc1nc(N)c2c(n1)CN(Cc1ccc(CN(CC)CC)c(C)c1)C(=O)N2] | ¹H NMR (400 MHz, MeOD) δ 7.48 (d, J = 7.8 Hz, 1H), 7.36-7.31 (m, 2H), 4.65 (s, 2H), 4.48 (t, J = 6.5 Hz, 2H), 4.41 (s, 2H), 4.37 (s, 2H), 3.29-3.23 (m, 4H), 2.47 (s, 3H), 1.81-1.73 (m, 2H), | 427.2 |
| | | 1.47 (dd, J = 15.0, 7.5 Hz, | |
| | | 2H), 1.37 (t, J = 7.3 Hz, 6H), | |
| | | 0.97 (t, J = 7.4 Hz, 3H). | |
| 70 | 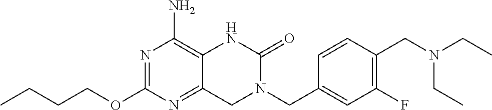 [SMILES: CCCCOc1nc(N)c2c(n1)CN(Cc1ccc(CN(CC)CC)c(F)c1)C(=O)N2] | ¹H NMR (400 MHz, MeOD) δ 7.66-7.60 (m, 1H), 7.36- 7.30 (m, 2H), 4.69 (s, 2H), 4.52-4.45 (m, 4H), 4.42 (s, 2H), 3.28-3.21 (m, 4H), 1.82- 1.74 (m, 2H), 1.47 (dq, J = 14.7, 7.4 Hz, 2H), 1.38 (t, J = | 431.2 |
| | | 7.3 Hz, 6H), 0.98 (t, J = 7.4 | |
| | | Hz, 3H). | |
| 71 | 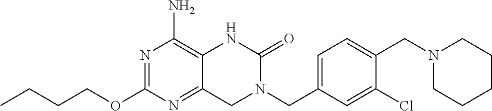 [SMILES: CCCCOc1nc(N)c2c(n1)CN(Cc1ccc(CN3CCCCC3)c(Cl)c1)C(=O)N2] | ¹H NMR (400 MHz, MeOD) δ 7.75 (d, J = 7.4 Hz, 1H), 7.60 (s, 1H), 7.47 (d, J = 7.3 Hz, 1H), 4.68 (s, 2H), 4.49 (d, J = 6.8 Hz, 6H), 3.50 (d, J = 11.7 Hz, 2H), 3.12 (t, J = 11.4 Hz, 2H), 1.94 (d, J = 12.0 Hz, 2H), 1.84-1.74 (m, 5H), 1.58- 1.43 (m, 3H), 0.98 (t, J = 7.4 Hz, 3H). | 459.2 |
| 72 | 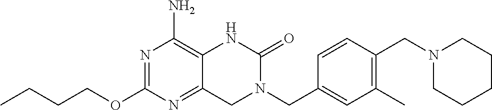 [SMILES: CCCCOc1nc(N)c2c(n1)CN(Cc1ccc(CN3CCCCC3)c(C)c1)C(=O)N2] | ¹H NMR (400 MHz, DMSO- d6) δ 9.81 (s, 1H), 8.85 (s, 1H), 7.61 (d, J = 7.6 Hz, 1H), 7.22 (d, J = 8.3 Hz, 2H), 4.53 (s, 2H), 4.27-4.20 (m, 4H), 4.17 (t, J = 6.5 Hz, 2H), 3.30 (d, J = 11.1 Hz, 2H), 2.98 (s, | 439.4 |
| | | 2H), 2.41 (s, 3H), 1.77 (s, | |
| | | 4H), 1.70-1.58 (m, 3H), 1.36 | |
| | | (dd, J = 14.9, 7.5 Hz, 3H), | |
| | | 0.90 (t, J = 7.4 Hz, 3H). | |
| 73 | 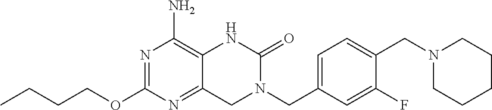 [SMILES: CCCCOc1nc(N)c2c(n1)CN(Cc1ccc(CN3CCCCC3)c(F)c1)C(=O)N2] | ¹H NMR (400 MHz, MeOD) δ 7.60 (t, J = 7.7 Hz, 1H), 7.32 (t, J = 9.1 Hz, 2H), 4.68 (s, 2H), 4.49-4.39 (m, 4H), 4.36 (s, 2H), 3.49 (d, J = 12.7 Hz, 2H), 3.03 (t, J = 12.4 Hz, 2H), 1.95 (d, J = 14.4 Hz, 2H), 1.88-1.65 (m, 5H), 1.58- 1.39 (m, 3H), 0.97 (t, J = 7.4 Hz, 3H). | 443.2 |
| 74 | 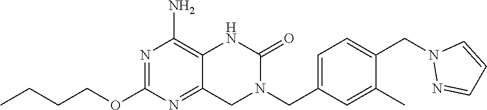 [SMILES: CCCCOc1nc(N)c2c(n1)CN(Cc1ccc(Cn3cccn3)c(C)c1)C(=O)N2] | ¹H NMR (400 MHz, DMSO- d6) δ 7.71 (d, J = 2.2 Hz, 1H), 7.46 (d, J = 1.3 Hz, 1H), 7.15- 7.07 (m, 2H), 6.90 (d, J = 7.9 Hz, 1H), 6.27 (t, J = 2.0 Hz, 1H), 5.32 (s, 2H), 4.47 (s, 2H), 4.13-4.05 (m, 4H), 2.28 | 422.2 |
| | | (s, 3H), 1.63-1.54 (m, 2H), | |
| | | 1.38-1.27 (m, 2H), 0.88 (t, J = | |
| | | 7.4 Hz, 3H). | |
| 75 | 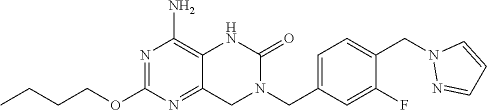 [SMILES: CCCCOc1nc(N)c2c(n1)CN(Cc1ccc(Cn3cccn3)c(F)c1)C(=O)N2] | ¹H NMR (400 MHz, DMSO- d6) δ 9.01 (s, 1H), 7.80 (d, J = 2.1 Hz, 1H), 7.45 (d, J = 1.4 Hz, 1H), 7.20-7.10 (m, 3H), 6.26 (t, J = 2.0 Hz, 1H), 5.37 (s, 2H), 4.53 (s, 2H), 4.26 (t, J = 6.5 Hz, 4H), 1.64 | 426.1 |
| | | (dd, J = 14.7, 6.8 Hz, 2H), | |
| | | 1.37 (dd, J = 15.0, 7.4 Hz, | |
| | | 2H), 0.90 (t, J = 7.4 Hz, 3H). | |
| 76 | 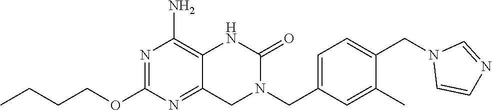 [SMILES: CCCCOc1nc(N)c2c(n1)CN(Cc1ccc(Cn3ccnc3)c(C)c1)C(=O)N2] | ¹H NMR (400 MHz, MeOD) δ 8.93 (s, 1H), 7.57 (d, J = 17.9 Hz, 2H), 7.30 (s, 1H), 7.28 (d, J = 1.0 Hz, 2H), 5.49 (s, 2H), 4.62 (s, 2H), 4.31- 4.30 (m, 4H), 2.32 (s, 3H), 1.76-1.69 (m, 2H), 1.45 (dd, | 422.2 |
| | | J = 15.1, 7.5 Hz, 2H), 0.96 (t, | |
| | | J = 7.4 Hz, 3H). | |
| 77 | 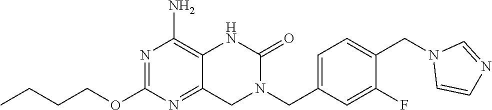 [SMILES: CCCCOc1nc(N)c2c(n1)CN(Cc1ccc(Cn3ccnc3)c(F)c1)C(=O)N2] | | 426.3 |
| 78 | 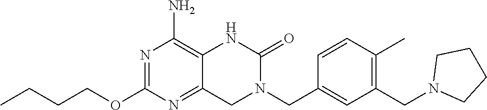 [SMILES: CCCCOc1nc(N)c2c(n1)CN(Cc1ccc(C)c(CN3CCCC3)c1)C(=O)N2] | ¹H NMR (400 MHz, DMSO- d₆) δ 10.43 (s, 1H), 8.86 (s, 1H), 7.58 (d, J = 7.5 Hz, 1H), 7.21 (d, J = 8.7 Hz, 2H), 4.52 (s, 2H), 4.34 (d, J = 5.8 Hz, 2H), 4.24-4.15 (m, 4H), 3.40 (d, J = 5.2 Hz, 2H), 3.11 | 425.3 |
| | | (d, J = 7.2 Hz, 2H), 2.42 (s, | |
| | | 3H), 2.02 (s, 2H), 1.96-1.83 | |
| | | (m, 2H), 1.68-1.56 (m, 2H), | |
| | | 1.36 (dd, J = 14.9, 7.4 Hz, | |
| | | 2H), 0.90 (t, J = 7.4 Hz, 3H). | |
| 79 | 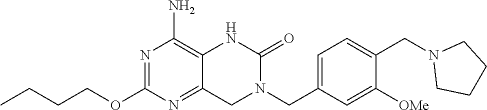 [SMILES: CCCCOc1nc(N)c2c(n1)CN(Cc1ccc(CN3CCCC3)c(OC)c1)C(=O)N2] | ¹H NMR (400 MHz, DMSO- d6) δ 10.46 (s, 1H), 9.18 (s, 1H), 8.46 (s, 2H), 7.55 (d, J = 7.7 Hz, 1H), 7.05 (s, 1H), 6.95 (d, J = 7.5 Hz, 1H), 4.58 (s, 2H), 4.40-4.22 (m, 6H), 3.86 (s, 3H), 3.44-3.24 (m, 2H), 3.17-2.96 (m, 2H), 2.08- 1.94 (m, 2H), 1.93-1.83 (m, 2H), 1.75-1.58 (m, 2H), 1.38 (m, 2H), 0.91 (t, J = 7.4 Hz, 3H). | 441.1 |
| 80 | 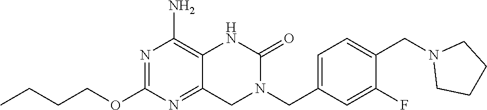 [SMILES: CCCCOc1nc(N)c2c(n1)CN(Cc1ccc(CN3CCCC3)c(F)c1)C(=O)N2] | ¹H NMR (400 MHz, MeOD) δ 7.62 (t, J = 7.5 Hz, 1H), 7.32 (s, 2H), 4.69 (s, 2H), 4.47 (d, J = 2.8 Hz, 6H), 3.55 (s, 2H), 3.23 (s, 2H), 2.19 (s, 2H), 2.03 (d, J = 6.8 Hz, 2H), 1.85-1.69 (m, 2H), 1.47 (d, J = | 429.2 |
| | | 7.5 Hz, 2H), 0.98 (t, J = 7.4 | |
| | | Hz, 3H). | |
| 81 | 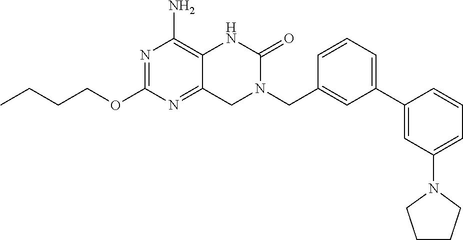 [SMILES: CCCCOc1nc(N)c2c(n1)CN(Cc1cccc(-c3cccc(N4CCCC4)c3)c1)C(=O)N2] | ¹H NMR (400 MHz, DMSO- d6): δ 9.01 (s, 1H), 8.15 (s, 2H), 7.67-7.53 (m, 2H), 7.44 (t, J = 7.9 Hz, 1H), 7.28 (m, 2H), 6.88 (d, J = 7.3 Hz, 1H), 6.78 (s, 1H), 6.60 (d, J = 7.0 Hz, 1H), 4.62 (s, 2H), 4.29- 4.24 (m, 4H), 3.41-3.13 (m, 4H), 2.08-1.87 (m, 4H), 1.75- 1.54 (m, 2H), 1.36 (m, 2H), 0.89 (t, J = 7.4 Hz, 3H). | 473.1 |
| 82 | 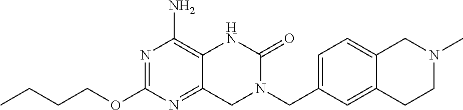 [SMILES: CCCCOc1nc(N)c2c(n1)CN(Cc1ccc3c(c1)CCN(C)C3)C(=O)N2] | ¹H NMR (400 MHz, DMSO- d₆): δ10.82 (s, 1H), 8.88 (s, 1H), 7.21-7.18 (m, 3H), 4.51- 4.48 (m, 3H), 4.22-4.19 (m, 5H), 3.62-3.59 (m, 1H), 3.28- 3.03 (m, 3H), 2.89 (s, 3H), 1.62-1.59 (m, 2H), 1.36-1.34 | 399.4 |
| | | (m, 2H), 0.98 (t, J = 7.4 Hz, | |
| | | 3H). | |
| 83 | 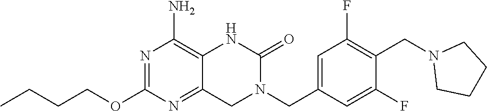 [SMILES: CCCCOc1nc(N)c2c(n1)CN(Cc1cc(F)c(CN3CCCC3)c(F)c1)C(=O)N2] | ¹H NMR (400 MHz, DMSO- d₆) δ 10.75 (s, 1H), 9.04 (s, 1H), 7.95 (s, 2H), 7.21 (d, J = 8.5 Hz, 2H), 4.59 (s, 2H), 4.42-4.38 (m, 2H), 4.33 (s, 2H), 4.25-4.20 (m, 2H), 3.51- 3.45 (m, 2H), 3.13-3.05 (m, | 447.7 |
| | | 2H), 2.06-1.97 (m, 2H), 1.93- | |
| | | 1.82 (m, 2H), 1.68-1.60 (m, | |
| | | 2H), 1.42-1.33 (m, 2H), 0.91 | |
| | | (t, J = 7.4 Hz, 3H). | |
| 84 | 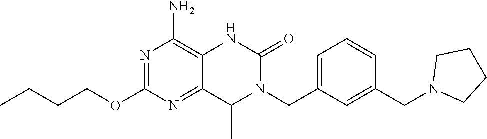 [SMILES: CCCCOc1nc(N)c2c(n1)C(C)N(Cc1cccc(CN3CCCC3)c1)C(=O)N2] | ¹H NMR (400 MHz, DMSO- d₆) δ 11.09 (s, 1H), 9.23 (s, 1H), 8.34 (s, 2H), 7.53 (d, J = 7.6 Hz, 2H), 7.43 (t, J = 7.5 Hz, 1H), 7.37 (d, J = 7.6 Hz, 1H), 5.02 (d, J = 15.6 Hz, 1H), 4.41-4.20 (m, 6H), 3.42- 3.20 (m, 2H), 3.11-2.85 (m, 2H), 2.09-1.92 (m, 2H), 1.91- | 425.1 |
| | | 1.78 (m, 2H), 1.74-1.57 (m, | |
| | | 2H), 1.47-1.23 (m, 5H), 0.91 | |
| | | (t, J = 7.4 Hz, 3H). | |
| 85 | 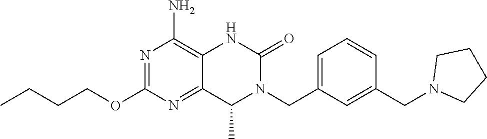 [SMILES: CCCCOc1nc(N)c2c(n1)[C@@H](C)N(Cc1cccc(CN3CCCC3)c1)C(=O)N2] | ¹H NMR (400 MHz, MeOD) δ 7.56 (s, 1H), 7.50-7.43 (m, 3H), 5.03 (d, J = 15.6 Hz, 1H), 4.55-4.39 (m, 4H), 4.37 (s, 2H), 3.53-3.40 (m, 2H), 3.22-3.09 (m, 2H), 2.23-2.11 (m, 2H), 2.07-1.92 (m, 2H), 1.83-1.72 (m, 2H), 1.55-1.35 (m, 5H), 0.98 (t, J = 7.4 Hz, | 425.1 |
| | | 3H). | |
| 86 | 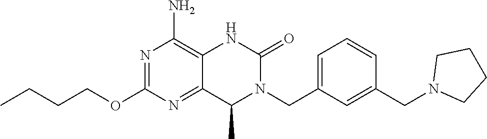 [SMILES: CCCCOc1nc(N)c2c(n1)[C@H](C)N(Cc1cccc(CN3CCCC3)c1)C(=O)N2] | ¹H NMR (400 MHz, MeOD) δ 7.62 (s, 1H), 7.47 (s, 3H), 5.04 (d, J = 15.6 Hz, 1H), 4.57 (q, J = 6.5 Hz, 1H), 4.49 (t, J = 6.5 Hz, 2H), 4.41 (d, J = 15.6 Hz, 1H), 4.38 (s, 2H), 3.59-3.40 (m, 2H), 3.23-3.07 (m, 2H), 2.24-2.10 (m, 2H), 2.09-1.95 (m, 2H), 1.82-1.70 | 425.3 |
| | | (m, H), 1.54-1.33 (m, 5H), | |
| | | 0.98 (t, J = 7.4 Hz, 3H). | |
| 87 | 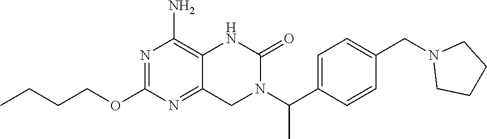 [SMILES: CCCCOc1nc(N)c2c(n1)CN(C(C)c1ccc(CN3CCCC3)cc1)C(=O)N2] | ¹H NMR (400 MHz, MeOD): δ 7.60 (d, J = 7.8 Hz, 2H), 7.52 (d, J = 7.8 Hz, 2H), 5.85 (q, J = 6.9 Hz, 1H), 4.50-4.37 (m, 5H), 3.97-3.93 (m, 1H), 3.50 (s, 2H), 3.20-3.18 (m, 2H), 2.18 (s, 2H), 2.08-1.97 (m, 2H), 1.80-1.72 (m, 2H), 1.64 (d, J = 7.0 Hz, 3H), | 425.3 |
| | | 1.50-1.41 (m, 2H), 0.97 (t, J = | |
| | | 7.3 Hz, 3H). | |
| 88 | 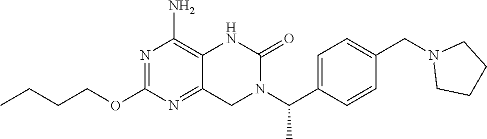 [SMILES: CCCCOc1nc(N)c2c(n1)CN([C@@H](C)c1ccc(CN3CCCC3)cc1)C(=O)N2] | ¹H NMR (400 MHz, MeOD) δ 7.36 (s, 4H), 5.83 (q, J = 7.1 Hz, 1H), 4.22-4.13 (m, 3H), 3.73-3.60 (m, 3H), 2.55 (m, 4H), 1.84-1.77 (m, 4H), 1.69- 1.58 (m, 5H), 1.47-1.39 (m, 2H), 0.93 (t, J = 7.4 Hz, 3H). | 425.4 |
| 89 | 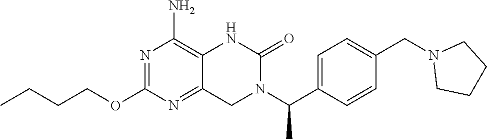 [SMILES: CCCCOc1nc(N)c2c(n1)CN([C@H](C)c1ccc(CN3CCCC3)cc1)C(=O)N2] | ¹H NMR (400 MHz, MeOD) δ 7.36 (s, 4H), 5.83 (q, J = 7.1 Hz, 1H), 4.24-4.12 (m, 3H), 3.71 (d, J = 16.0 Hz, 1H), 3.68-3.60 (m, 2H), 2.55 (s, 4H), 1.80 (s, 4H), 1.70-1.58 (m, 5H), 1.40 (m, 2H), 0.93 (t, J = 7.4 Hz, 3H). | 425.4 |
| 90 | 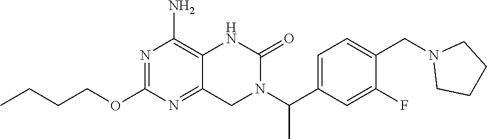 [SMILES: CCCCOc1nc(N)c2c(n1)CN(C(C)c1ccc(CN3CCCC3)c(F)c1)C(=O)N2] | ¹H NMR (400 MHz, DMSO- d6) δ 8.59 (s, 1H), 7.39 (t, J = 8.0 Hz, 1H), 7.21-7.03 (m, 2H), 6.85 (s, 2H), 5.69 (d, J = 7.1 Hz, 1H), 4.19 (d, J = 15.8 Hz, 1H), 4.04 (t, J = 6.6 Hz, 2H), 3.64 (s, 1H), 3.60 (s, 2H), 2.44 (s, 4H), 1.67 (s, 4H), 1.61-1.54 (m, 2H), 1.52 | 443.3 |
| | | (t, J = 6.6 Hz, 3H), 1.34 (dd, | |
| | | J = 14.9, 7.4 Hz, 2H), 0.88 (t, | |
| | | J = 7.4 Hz, 3H). | |
| 91 | 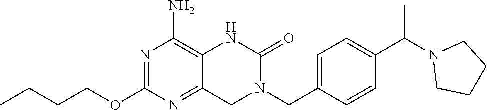 [SMILES: CCCCOc1nc(N)c2c(n1)CN(Cc1ccc(C(C)N3CCCC3)cc1)C(=O)N2] | ¹H NMR (400 MHz, DMSO- d₆) δ 7.34 (q, J = 7.9 Hz, 4H), 4.67-4.53 (m, 3H), 4.17 (d, J = 8.9 Hz, 4H), 2.64 (d, J = 9.0 Hz, 2H), 2.40 (d, J = 7.8 Hz, 2H), 1.79 (s, 4H), 1.71- 1.64 (m, 2H), 1.44 (dd, J = | 425.3 |
| | | 14.2, 7.1 Hz, 5H), 0.94 (t, J = | |
| | | 7.3 Hz, 3H). | |
| 92 | 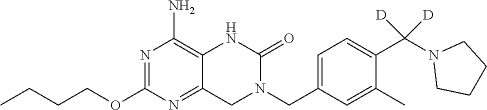 [SMILES: [2H]C([2H])(c1ccc(CN2Cc3nc(OCCCC)nc(N)c3NC2=O)cc1C)N1CCCC1] | ¹H NMR (400 MHz, MeOD) δ 7.49 (d, J = 7.8 Hz, 1H), 7.34-7.29 (m, 2H), 4.63 (s, 2H), 4.40 (t, J = 6.5 Hz, 2H), 4.35 (s, 2H), 3.54 (s, 2H), 3.24 (s, 2H), 2.47 (s, 3H), 2.20 (s, 2H), 2.08-2.00 (m, | 427.3 |
| | | 2H), 1.78-1.72 (m, 2H), | |
| | | 1.50-1.42 (m, 2H), 0.97 (t, J = | |
| | | 7.4 Hz, 3H). | |
| 93 | 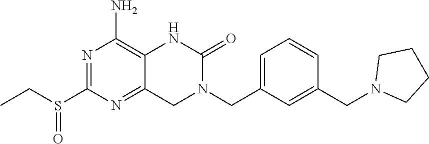 [SMILES: CCS(=O)c1nc(N)c2c(n1)CN(Cc1cccc(CN3CCCC3)c1)C(=O)N2] | ¹H NMR (400 MHz, MeOD): δ 7.42-7.23 (m, 4H), 4.65 (s, 2H), 4.37 (s, 2H), 3.70 (s, 2H), 2.98-2.96 (m, 2H), 2.60 (s, 4H), 1.81 (s, 4H), 1.19 (t, J = 7.4 Hz, 3H). | 415.3 |
| 94 | 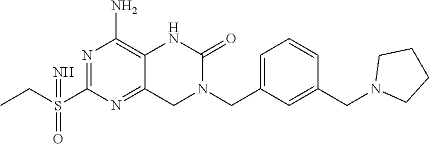 [SMILES: CCS(=N)(=O)c1nc(N)c2c(n1)CN(Cc1cccc(CN3CCCC3)c1)C(=O)N2] | ¹H NMR (400 MHz, MeOD): δ 7.32 (dd, J = 20.8, 10.6 Hz, 4H), 4.65 (s, 2H), 4.38 (s, 2H), 3.68 (s, 2H), 3.45 (dd, J = 17.0, 9.5 Hz, 2H), 2.58 (s, 4H), 1.81 (s, 4H), 1.28 (t, J = 7.4 Hz, 3H). | 430.2 |

### Example 95: Preparation of 4-amino-7-(S-methyl sulfonimidoyl)-1-(4-(pyrrolidin-1-ylmethyl)benzyl)-1,3-dihydro-2H-imidazo[4,5-c]quinolin-2-one

The compound of Example 95 (0.021 g, yield: 15.8%) was prepared by referring to the preparation method of Example 1. LC-MS (ESI) [M+H]⁺=451.0; ¹H NMR (400 MHz, DMSO-d₆): δ 7.99 (d, J=1.6 Hz, 1H), 7.83 (d, J=8.8 Hz, 1H), 7.43 (d, J=7.2 Hz, 1H), 7.23 (d, J=8.0 Hz, 2H), 7.17 (d, J=8.0 Hz, 2H), 6.53 (s, 2H), 5.44 (s, 2H), 4.07 (s, 1H), 3.49 (s, 2H), 3.03 (s, 3H), 2.41-2.30 (m, 4H), 1.70-1.58 (m, 4H).

### Example 96: Preparation of 2-butoxy-8-(difluoromethyl)-9-(3-(pyrrolidin-1-ylmethyl)benzyl)-9H-purin-6-amine

### Step 1: Preparation of 8-bromo-2-butoxy-9-(tetrahydro-2H-pyran-2-yl)-9H-purin-6-amine

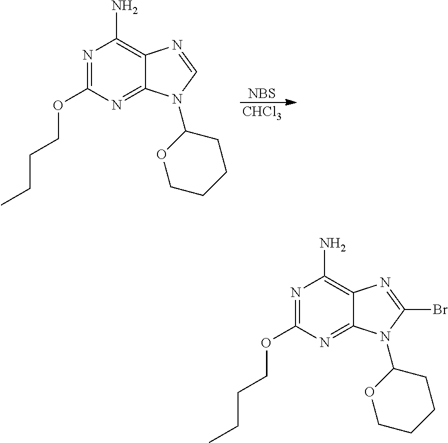

2-butoxy-9-(tetrahydro-2H-pyran-2-yl)-9H-purin-6-amine (2.05 g, 7.04 mmol, 1.0 eq) was dissolved in chloroform (10 mL). 1-bromopyrrolidine-2,5-dione (3.76 g, 21.1 mmol, 3.0 eq) was added. The reaction mixture was stirred at 25° C. for 18 h. After the reaction was completed, as detected by LC-MS, the reaction mixture was concentrated to remove the solvent. Water (30 mL) was added. The mixture was extracted with dichloromethane (25 mL×2). The organic phases were combined, dried over anhydrous sodium sulfate, filtered and concentrated to give a crude product, which was separated and purified by flash chromatography (DCM:MeOH=100:0 to 93:7) to give the target compound (1.50 g, yield: 51.2%). LC-MS (ESI) [M+H]⁺=369.92; ¹H NMR (400 MHz, CDCl₃): δ 5.64-5.58 (m, 1H), 4.33-4.29 (m, 2H), 4.15-4.14 (m, 1H), 3.73-3.69 (m, 1H), 3.05-3.02 (m, 1H), 2.03-1.91 (m, 1H), 1.81-1.47 (m, 10H), 0.99-0.95 (m, 3H).

### Step 2: Preparation of methyl 6-amino-2-butoxy-9-(tetrahydro-2H-pyran-2-yl)-9H-purin-8-carboxylate

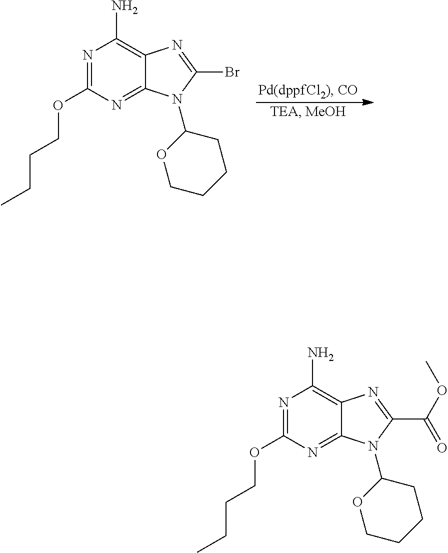 [SMILES: CCCCOc1nc(N)c2nc(Br)n(C3CCCCO3)c2n1 ; "Pd(dppfCl2), CO" ; "TEA, MeOH" ; CCCCOc1nc(N)c2nc(C(=O)OC)n(C3CCCCO3)c2n1]

8-bromo-2-butoxy-9-(tetrahydro-2H-pyran-2-yl)-9H-purin-6-amine (1.50 g, 4.05 mmol, 1.0 eq) was dissolved in absolute methanol (10 mL). [1,1′-bis(diphenylphosphino)ferrocene]dichloropalladium(II) (296 mg, 405 μmol, 0.1 eq) and triethylamine (2.05 g, 20.3 mmol, 2.82 mL, 5.0 eq) were added. The reaction mixture was purged with nitrogen 3 times and then purged with carbon monoxide 3 times. The reaction mixture was stirred at 80° C. for 18 h under carbon monoxide atmosphere (40 Psi). After the reaction was completed, as detected by LC-MS, the reaction mixture was concentrated to remove the solvent. Water (30 mL) was added to the reaction mixture. The mixture was extracted with ethyl acetate (25 mL×2). The organic phases were combined, dried over anhydrous sodium sulfate, filtered and concentrated to give a crude product, which was separated and purified by flash chromatography (DCM:MeOH=100:0 to 93:7) to give the target compound (1.07 g, yield: 75.6%). LC-MS (ESI) [M+H]⁺=350.01; ¹H NMR (400 MHz, CDCl₃): δ 4.38-4.35 (m, 2H), 4.34-4.32 (m, 1H), 4.02-4.00 (m, 2H), 3.67-3.64 (m, 1H), 3.48 (s, 3H), 3.20-3.19 (m, 1H), 1.83-1.52 (m, 10H), 0.99-0.95 (m, 3H).

### Step 3: Preparation of (6-amino-2-butoxy-9-(tetrahydro-2H-pyran-2-yl)-9H-purin-8-yl)methanol

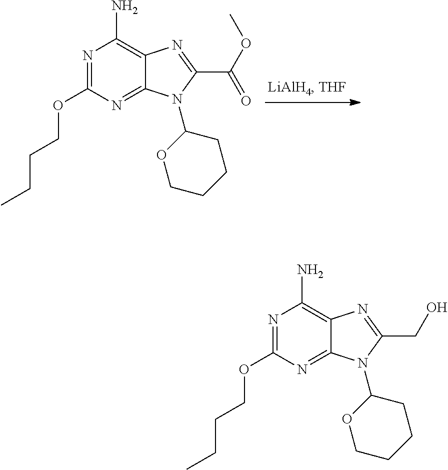 [SMILES: CCCCOc1nc(N)c2nc(C(=O)OC)n(C3CCCCO3)c2n1 ; CCCCOc1nc(N)c2nc(CO)n(C3CCCCO3)c2n1]

Methyl 6-amino-2-butoxy-9-(tetrahydro-2H-pyran-2-yl)-9H-purin-8-carboxylate (1.07 g, 3.06 mmol, 1.0 eq) was dissolved in anhydrous tetrahydrofuran (10 mL). Lithium aluminum hydride (181 mg, 4.79 mmol, 1.5 eq) was added slowly at 0° C. The reaction mixture was stirred at 0-10° C. for 3 h. After the reaction was completed, as detected by LC-MS, the reaction mixture was added dropwise to sodium sulfate decahydrate (10 mL). The mixture was filtered and concentrated to give the target compound (1.01 g, yield: 98.5%), which was used directly in the next step. LC-MS (ESI) [M+H]⁺=322.08.

### Step 4: Preparation of 6-amino-2-butoxy-9-(tetrahydro-2H-pyran-2-yl)-9H-purin-8-carbaldehyde

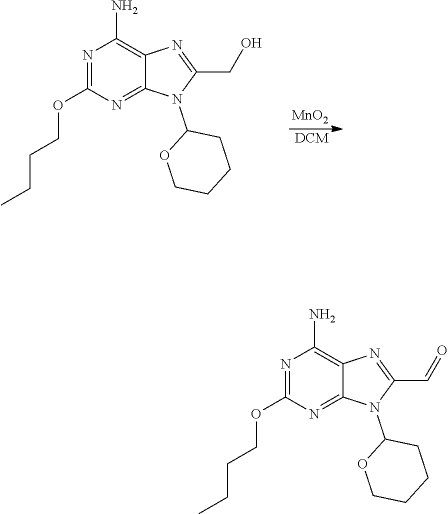 [SMILES: CCCCOc1nc(N)c2nc(CO)n(C3CCCCO3)c2n1 ; "MnO2" ; CCCCOc1nc(N)c2nc(C=O)n(C3CCCCO3)c2n1]

(6-amino-2-butoxy-9-(tetrahydro-2H-pyran-2-yl)-9H-purin-8-yl)methanol (1.01 g, 3.14 mmol, 1.0 eq) was dissolved in anhydrous dichloromethane (10 mL). Activated manganese dioxide (1.37 g, 15.7 mmol, 5.0 eq) was added. The reaction mixture was stirred at 40° C. for 3 h. After the reaction was completed, as detected by TLC (DCM:MeOH=10:1, 254 nm), the reaction mixture was filtered and concentrated to give a solid. The solid was separated and purified by flash chromatography (DCM:MeOH=100:0 to 91:9) to give the target compound (990 mg, yield: 98.6%).

### Step 5: Preparation of 2-butoxy-8-(difluoromethyl)-9-(tetrahydro-2H-pyran-2-yl)-9H-purin-6-amine

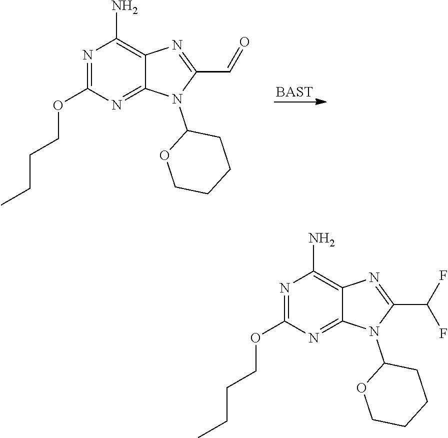 [SMILES: CCCCOc1nc(N)c2nc(C=O)n(C3CCCCO3)c2n1 ; CCCCOc1nc(N)c2nc(C(F)F)n(C3CCCCO3)c2n1]

6-amino-2-butoxy-9-(tetrahydro-2H-pyran-2-yl)-9H-purin-8-carbaldehyde (990 mg, 3.10 mmol, 1.0 eq) was dissolved in dichloromethane (10 mL). Bis(2-methoxyethyl)aminosulfur trifluoride (2.06 g, 9.30 mmol, 3.0 eq) was added at 0° C. The reaction mixture was stirred at 25° C. for 30 min. After the reaction was completed, as detected by LC-MS, the reaction mixture was slowly added dropwise into saturated sodium bicarbonate solution (30 mL) to quench the reaction. The mixture was extracted with dichloromethane (15 mL). The organic phase was washed with water (5 mL×2), dried over anhydrous sodium sulfate, filtered and concentrated to give a crude product, which was separated and purified by flash chromatography (DCM:MeOH=100:0 to 95:5) to give the target compound (270 mg, yield: 25.5%). LC-MS (ESI) [M+H]⁺=342.02; ¹H NMR (400 MHz, CDCl₃): δ 7.14-6.88 (m, 1H), 5.78 (d, J=13.2 Hz, 1H), 5.61 (s, 2H), 4.35-4.31 (m, 2H), 3.72-3.71 (m, 2H), 3.38-3.35 (m, 2H), 2.28-2.19 (m, 1H), 2.04-1.89 (m, 2H), 1.80-1.71 (m, 4H), 1.53-1.48 (m, 3H), 0.99-0.96 (m, 3H).

### Step 6: Preparation of 2-butoxy-8-(difluoromethyl)-9H-purin-6-amine

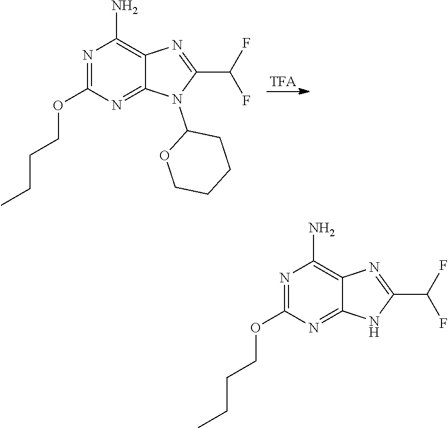 [SMILES: CCCCOc1nc(N)c2nc(C(F)F)n(C3CCCCO3)c2n1 ; CCCCOc1nc(N)c2nc(C(F)F)[nH]c2n1]

2-butoxy-8-(difluoromethyl)-9-(tetrahydro-2H-pyran-2-yl)-9H-purin-6-amine (270 mg, 790 μmol, 1.0 eq) was dissolved in dichloromethane (4 mL), and then trifluoroacetic acid (2 mL) was added. The reaction mixture was stirred at 25° C. for 1 h. After the reaction was completed, as detected by LC-MS, the reaction mixture was slowly added dropwise into saturated sodium bicarbonate solution (10 mL). The mixture was extracted with dichloromethane (10 mL×2). The organic phase was washed with water (10 mL×2), dried over anhydrous sodium sulfate, filtered and concentrated to give the target compound (200 mg, yield: 84.5%), which was used directly in the next step. LC-MS (ESI) [M+H]⁺=258.06.

### Step 7: Preparation of 2-butoxy-8-(difluoromethyl)-9-(3-(pyrrolidin-1-ylmethyl)benzyl)-9H-purin-6-amine

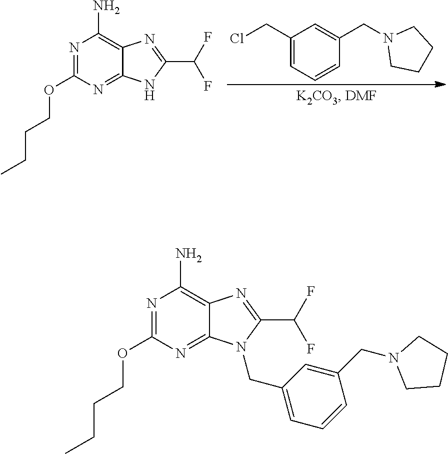

2-butoxy-8-(difluoromethyl)-9H-purin-6-amine (200 mg, 777 μmol, 1.0 eq) was dissolved in anhydrous N,N-dimethylformamide (10 mL), and then 1-(3-(chloromethyl)benzyl)pyrrolidine (163 mg, 777 μmol, 1.0 eq) and potassium carbonate (322 mg, 2.33 mmol, 3.0 eq) were added sequentially. The mixture was stirred at 25° C. for 18 h. After the reaction was completed, as detected by LC-MS, water (20 mL) was added to the mixture. The mixture was extracted with ethyl acetate (20 mL×2). The organic phases were combined, washed with saturated brine (15 mL×2), dried over anhydrous sodium sulfate, filtered and concentrated to give a crude product, which was separated and purified by Prep-HPLC (0.01% aqueous formic acid, MeCN) to give the target compound (12.73 mg, yield: 3.80%). LC-MS (ESI) [M+H]⁺=431.2; ¹H NMR (400 MHz, MeOD): δ 7.43-7.38 (m, 2H), 7.32-7.30 (m, 2H), 7.09-6.83 (m, 1H), 5.52 (s, 2H), 4.32 (t, J=6.8 Hz, 2H), 4.07 (s, 2H), 2.99 (brs, 4H), 1.97-1.94 (m, 4H), 1.75-1.71 (m, 2H), 1.50-1.46 (m, 2H), 0.96 (t, J=7.4 Hz, 3H).

### Example 97: Preparation of (3-((4-chloro-1H-imidazo[4,5-c]quinolin-1-yl)methyl)phenyl)(pyrrolidin-1-yl)methanone

The compound of Example 97 was prepared by referring to Step 5 of the preparation method of Example 3.

### Example 98: Preparation of 4-chloro-1-(3-(pyrrolidin-1-ylmethyl)benzyl)-1H-imidazo[4,5-c]quinoline

The compound of Example 98 was prepared by referring to Step 1 of the preparation method of Example 2.

### Example 99: Preparation of ethyl 4-chloro-1-(3-(pyrrolidin-1-ylmethyl)benzyl)-1H-imidazo[4,5-c]quinolin-2-carboxylate

### Step 1: Preparation of 2-chloro-3-nitro-N-(3-(pyrrolidin-1-ylmethyl)benzyl)quinoline-4-amine

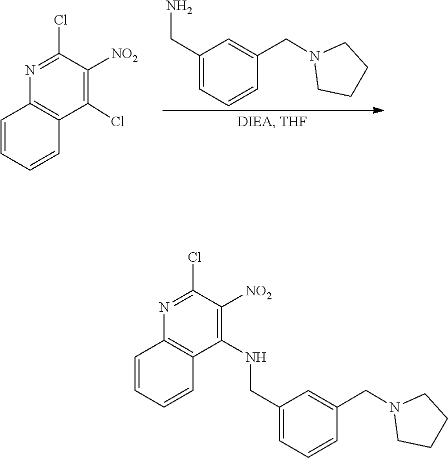 [SMILES: O=[N+]([O-])c1c(Cl)nc2ccccc2c1Cl ; NCc1cccc(CN2CCCC2)c1 ; O=[N+]([O-])c1c(Cl)nc2ccccc2c1NCc1cccc(CN2CCCC2)c1]

2,4-dichloro-3-nitroquinoline (3.50 g, 14.4 mmol, 1.0 eq) and (3-(pyrrolidin-1-ylmethyl)phenyl)methylamine (2.74 g, 14.4 mmol, 1.0 eq) were dissolved in tetrahydrofuran (40 mL). DIEA (2.42 g, 18.7 mmol, 1.3 eq) was added. The reaction mixture was stirred at 25° C. for 16 h. After the reaction was completed, as detected by TLC (DCM:MeOH=10:1, 254 nm), the reaction mixture was added to water (50 mL). The mixture was extracted with ethyl acetate (50 mL×2). The organic phases were combined, washed with saturated brine, dried over anhydrous sodium sulfate, filtered and concentrated to give a crude product, which was separated and purified by flash chromatography (silica gel, PE:EA=100:0 to 91:9) to give the target compound (5.00 g, yield: 78.7%). LC-MS (ESI) [M+H]⁺=397.7.

### Step 2: Preparation of 2-chloro-N⁴-(3-(pyrrolidin-1-ylmethyl)benzyl)quinolin-3,4-diamine

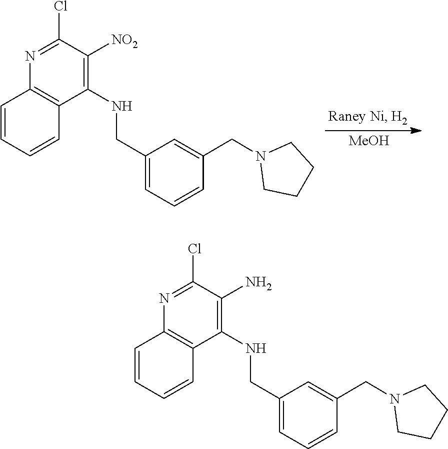

2-chloro-3-nitro-N-(3-(pyrrolidin-1-ylmethyl)benzyl)quinolin-4-amine (5.00 g, 12.6 mmol, 1.0 eq) was dissolved in methanol (50 mL). Raney nickel (1.08 g) was added under nitrogen atmosphere. The reaction mixture was purged with nitrogen 3 times and then purged with hydrogen 3 times. The reaction mixture was stirred at 25° C. for 2 h under hydrogen atmosphere (15 Psi). After the reaction was completed, as detected by TLC (DCM:MeOH=10:1, 254 nm), the mixture was filtered through celite and concentrated to give the target compound (2.10 g, yield: 40.9%). LC-MS (ESI) [M+H]⁺=367.07.

### Step 3: Preparation of ethyl 4-chloro-1-(3-(pyrrolidin-1-ylmethyl)benzyl)-1H-imidazo[4,5-c]quinolin-2-carboxylate

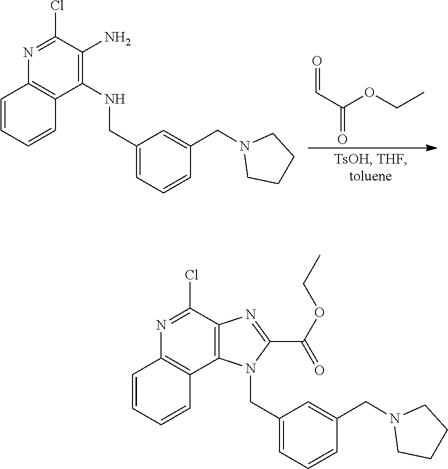

2-chloro-N⁴-(3-(pyrrolidin-1-ylmethyl)benzyl)quinolin-3,4-diamine (480 mg, 1.31 mmol, 1.0 eq) was dissolved in toluene (9 mL) and tetrahydrofuran (3 mL). A solution of ethyl glyoxylate (50%, 534 mg, 2.62 mmol, 2.0 eq) in toluene and p-toluenesulfonic acid (249 mg, 1.44 mmol, 1.1 eq) were added. The mixture was stirred at 100° C. for 1 h. After the reaction was completed, as detected by LC-MS, the reaction mixture was added with water (10 mL) for dilution and extracted with ethyl acetate (15 mL×3). The organic phases were combined, washed with saturated brine, dried over anhydrous sodium sulfate, filtered and concentrated to give a crude product, which was separated and purified by flash chromatography (silica gel, DCM:MeOH=100:0 to 94:6) to give the target compound (430 mg, yield: 72.2%). LC-MS (ESI) [M+H]⁺=449.0. ¹H NMR (400 MHz, DMSO-d₆): δ 8.32 (s, 1H), 8.16 (d, J=8.4 Hz, 1H), 8.11 (d, J=8.4 Hz, 1H), 7.79-7.75 (m, 1H), 7.59-7.55 (m, 1H), 7.27-7.23 (m, 1H), 7.16 (d, J=7.6 Hz, 1H), 7.08 (s, 1H), 6.97 (d, J=7.6 Hz, 1H), 6.36 (s, 2H), 4.46-4.40 (m, 2H), 3.48 (s, 2H), 2.25 (s, 4H), 1.56 (s, 4H), 1.35 (t, J=7.2 Hz, 1H).

### Example 100: Preparation of 4-chloro-1-(3-((2-oxopyrrolidin-1-yl)methyl)benzyl)-1H-imidazo[4,5-c]quinolin-2(3H)-one

The compound of Example 100 was prepared by referring to Step 5 of the preparation method of Example 9.

### Example 101: Preparation of 4-chloro-1-(3-(pyrrolidin-1-ylmethyl)benzyl)-1,3-dihydro-2H-imidazo[4,5-c]quinolin-2-one

The compound of Example 101 was prepared by referring to Step 5 of the preparation method of Example 1.

### Examples 102-126

The compounds of Examples 102-126 were prepared by referring to the preparation method of Example 10

| | | | LC-MS |
|---|---|---|---|
| | | | (ESI) |
| Example | Chemical structure | ¹H NMR | [M + H]⁺ |
| 102 | 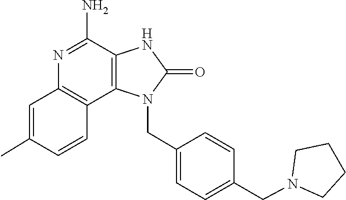 [SMILES: Cc1ccc2c(c1)nc(N)c1[nH]c(=O)n(Cc3ccc(CN4CCCC4)cc3)c12] | ¹H NMR (400 MHz, DMSO-d₆) δ 7.59 (d, J = 8.4 Hz, 1H), 7.28 (s, 1H), 7.22 (d, J = 8.1 Hz, 2H), 7.14 (d, J = 8.1 Hz, 2H), 6.83 (dd, J = 8.5, 1.5 Hz, 1H), 6.36 (s, 2H), 5.40 (s, 2H), 3.48 (s, 2H), 2.38-2.33 (m, 4H), 2.31 (s, 3H), 1.66-1.61 (m, 4H). | 410.2 |
| 103 | 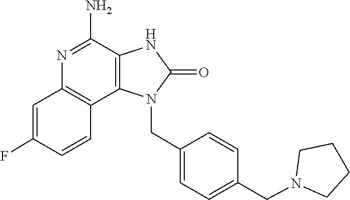 [SMILES: Nc1nc2cc(F)ccc2c2c1[nH]c(=O)n2Cc1ccc(CN2CCCC2)cc1] | ¹H NMR (400 MHz, MeOD) δ 8.02 (dd, J = 9.2, 5.4 Hz, 1H), 7.56 (d, J = 8.2 Hz, 2H), 7.51-7.41 (m, 3H), 7.23 (td, J = 9.1, 2.5 Hz, 1H), 5.68 (s, 2H), 4.37 (s, 2H), 3.48 (d, J = 8.8 Hz, 2H), 3.16 (d, J = 14.5 Hz, 2H), 2.17 (d, J = 14.3 Hz, 2H), 2.01 (d, J = 3.2 Hz, 2H). | 392.2 |
| 104 | 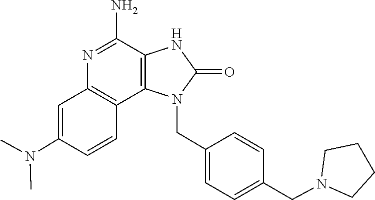 [SMILES: CN(C)c1ccc2c(c1)nc(N)c1[nH]c(=O)n(Cc3ccc(CN4CCCC4)cc3)c12] | ¹H NMR (400 MHz, DMSO-d₆) δ 13.42 (s, 1H), 11.93 (s, 1H), 10.73 (s, 1H), 8.88 (s, 1H), 8.27 (s, 2H), 7.64 (d, J = 9.4 Hz, 1H), 7.56 (d, J = 8.1 Hz, 2H), 7.32 (d, J = 8.1 Hz, 2H), 6.86 (dd, J = 9.4, 2.4 Hz, 1H), 6.75 (d, J = 2.4 Hz, 1H), 5.49 (s, 2H), 4.28 (d, J = 5.8 Hz, 2H), 3.28 (m, 2H), 3.08 (m, 2H), 2.99 (s, 6H), 1.97 (m, 2H), 1.84 (m, 2H). | 417.2 |
| 105 | 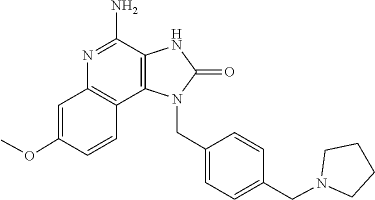 [SMILES: COc1ccc2c(c1)nc(N)c1[nH]c(=O)n(Cc3ccc(CN4CCCC4)cc3)c12] | ¹H NMR (400 MHz, DMSO-d₆) δ 13.85 (s, 1H), 12.15 (s, 1H), 10.66 (s, 1H), 8.49 (s, 2H), 7.78 (d, J = 9.2 Hz, 1H), 7.55 (d, J = 8.1 Hz, 2H), 7.33 (d, J = 8.0 Hz, 2H), 7.22 (d, J = 2.3 Hz, 1H), 6.99 (dd, J = 9.2, 2.4 Hz, 1H), 5.53 (s, 2H), 4.28 (d, J = 5.4 Hz, 2H), 3.84 (s, 3H), 3.32-3.20 (m, 2H), 3.00 (m, 2H), 2.10-1.91 (m, 2H), 1.91-1.76 (m, 2H). | 404.2 |
| 106 | 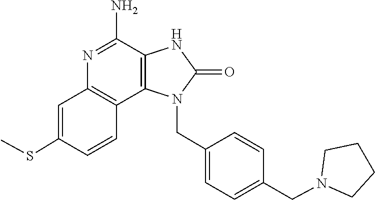 [SMILES: CSc1ccc2c(c1)nc(N)c1[nH]c(=O)n(Cc3ccc(CN4CCCC4)cc3)c12] | ¹H NMR (400 MHz, MeOD): δ 7.81 (d, J = 8.9 Hz, 1H), 7.53 (d, J = 8.2 Hz, 2H), 7.48 (d, J = 1.8 Hz, 1H), 7.42 (d, J = 8.2 Hz, 2H), 7.24 (dd, J = 8.9, 1.9 Hz, 1H), 5.64 (s, 2H), 4.35 (s, 2H), 3.45-3.43 (m, 2H), 3.16-3.13 (m, 2H), 2.57 (s, 3H), 2.15-2.13 (m, 2H), 2.05-1.88 (m, 2H). | 420.0 |
| 107 | 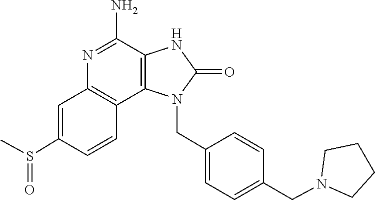 [SMILES: CS(=O)c1ccc2c(c1)nc(N)c1[nH]c(=O)n(Cc3ccc(CN4CCCC4)cc3)c12] | ¹H NMR (400 MHz, DMSO-d₆): δ 14.31 (s, 1H), 12.42 (s, 1H), 10.62 (s, 1H), 8.71 (s, 2H), 8.09 (s, 1H), 7.99 (d, J = 8.8 Hz, 1H), 7.57-7.55 (m, 3H), 7.36 (d, J = 8.0 Hz, 2H), 5.59 (s, 2H), 4.29 (d, J = 5.6 Hz, 2H), 3.34-3.23 (m, 2H), 3.06-2.94 (m, 2H), 2.80 (s, 3H), 2.04-1.93 (m, 2H), 1.90-1.77 (m, 2H). | 436.0 |
| 108 | 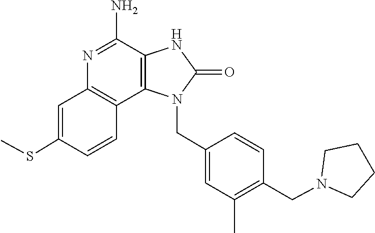 [SMILES: CSc1ccc2c(c1)nc(N)c1[nH]c(=O)n(Cc3ccc(CN4CCCC4)c(C)c3)c12] | ¹H NMR (400 MHz, DMSO-d₆) δ 13.83 (s, 1H), 12.15 (s, 1H), 10.16 (s, 1H), 8.51 (s, 2H), 7.75 (d, J = 8.9 Hz, 1H), 7.56-7.47 (m, 2H), 7.22 (m, 1H), 7.18 (s, 1H), 7.13 (d, J = 8.4 Hz, 1H), 5.48 (s, 2H), 4.31 (d, J = 5.6 Hz, 2H), 3.35-3.23 (m, 2H), 3.07 (m, 2H), 2.54 (s, 3H), 2.36 (s, 3H), 2.07-1.95 (m, 2H), 1.93-1.79 (m, 2H). | 434.1 |
| 109 | 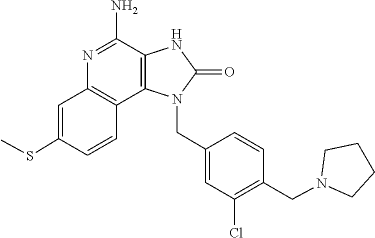 [SMILES: CSc1ccc2c(c1)nc(N)c1[nH]c(=O)n(Cc3ccc(CN4CCCC4)c(Cl)c3)c12] | ¹H NMR (400 MHz, DMSO-d₆) δ 13.84 (s, 1H), 12.18 (s, 1H), 10.45 (s, 1H), 8.49 (s, 2H), 7.76 (t, J = 7.3 Hz, 2H), 7.54 (d, J = 8.6 Hz, 2H), 7.30 (d, J = 7.6 Hz, 1H), 7.23 (d, J = 9.2 Hz, 1H), 5.54 (s, 2H), 4.46 (s, 2H), 3.40 (s, 2H), 3.10 (s, 2H), 2.54 (s, 3H), 2.01 (s, 2H), 1.87 (s, 2H). | 454.1 |
| 110 | 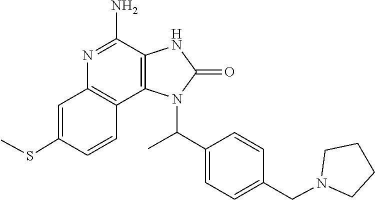 [SMILES: CSc1ccc2c(c1)nc(N)c1[nH]c(=O)n(C(C)c3ccc(CN4CCCC4)cc3)c12] | ¹H NMR (400 MHz, MeOD) δ 7.62- 7.47 (m, 6H), 7.15 (s, 1H), 6.39-6.34 (m, 1H), 4.38 (s, 2H), 3.45-3.43 (m, 2H), 3.17-3.14 (m, 2H), 2.56 (s, 3H), 2.16 (s, 2H), 2.16-2.00 (m, 5H). | 434.3 |
| 111 | 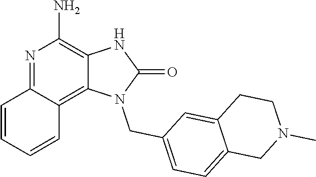 [SMILES: CN1CCc2cc(Cn3c(=O)[nH]c4c(N)nc5ccccc5c43)ccc2C1] | ¹H NMR (400 MHz, MeOD) δ 7.78 (d, J = 8.4 Hz, 1H), 7.60 (d, J = 8.4 Hz, 1H), 7.38 (t, J = 7.7 Hz, 1H), 7.11-7.05 (m, 3H), 7.02 (s, 1H), 5.51 (s, 2H), 3.65 (s, 2H), 2.89 (t, J = 5.7 Hz, 2H), 2.78 (t, J = 6.0 Hz, 2H), 2.47 (s, 3H). | 360.2 |
| 112 | 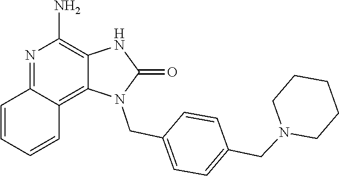 [SMILES: Nc1nc2ccccc2c2c1[nH]c(=O)n2Cc1ccc(CN2CCCCC2)cc1] | ¹H NMR (400 MHz, DMSO-d₆): δ 14.04 (s, 1H), 12.34 (s, 1H), 10.20 (s, 1H), 8.57 (s, 2H), 7.88 (d, J = 8.2 Hz, 1H), 7.77 (d, J = 8.4 Hz, 1H), 7.62 (t, J = 7.7 Hz, 1H), 7.55 (d, J = 8.1 Hz, 2H), 7.35 (m, 3H), 5.57 (s, 2H), 4.20 (d, J = 4.7 Hz, 2H), 3.22- 3.21 (m, 2H), 2.88-2.72 (m, 2H), 1.88-1.57 (m, 5H), 1.41-1.21 (m, 1H). | 388.3 |
| 113 | 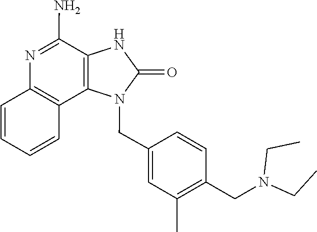 [SMILES: CCN(CC)Cc1ccc(Cn2c(=O)[nH]c3c(N)nc4ccccc4c32)cc1C] | ¹H NMR (400 MHz, DMSO-d₆) δ 10.99 (s, 1H), 7.73 (d, J = 8.3 Hz, 1H), 7.52 (d, J = 8.2 Hz, 1H), 7.31 (t, J = 7.2 Hz, 1H), 7.19 (d, J = 7.8 Hz, 1H), 7.08-7.00 (m, 2H), 6.95 (d, J = 8.0 Hz, 1H), 6.34 (s, 2H), 5.39 (s, 2H), 3.40 (s, 2H), 2.39 (q, J = 7.0 Hz, 4H), 2.25 (s, 3H), 0.92 (t, J = 7.1 Hz, 6H). | 390.2 |
| 114 | 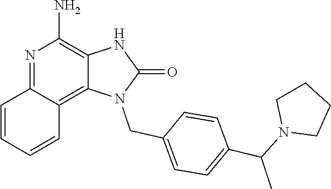 [SMILES: CC(c1ccc(Cn2c(=O)[nH]c3c(N)nc4ccccc4c32)cc1)N1CCCC1] | ¹H NMR (400 MHz, MeOD) δ 7.99 (d, J = 8.4 Hz, 1H), 7.76-7.66 (m, 2H), 7.52 (d, J = 8.3 Hz, 2H), 7.47- 7.35 (m, 3H), 5.68 (s, 2H), 4.36 (dd, J = 14.9, 5.8 Hz, 1H), 3.75 (t, J = 9.1 Hz, 1H), 3.21 (d, J = 11.9 Hz, 1H), 3.04-2.88 (m, 2H), 2.20-2.08 (m, 1H), 2.03 (t, J = 9.6 Hz, 2H), 1.89 (d, J = 8.3 Hz, 1H), 1.69 (d, J = 6.8 Hz, 3H). | 388.1 |
| 115 | 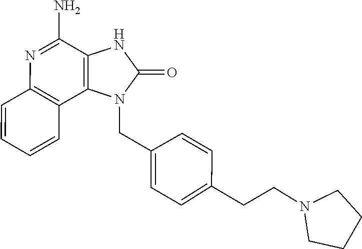 [SMILES: Nc1nc2ccccc2c2c1[nH]c(=O)n2Cc1ccc(CCN2CCCC2)cc1] | ¹H NMR (400 MHz, DMSO-d6) δ 13.99 (s, 1H), 12.28 (s, 1H), 10.44 (s, 1H), 8.53 (s, 2H), 7.91 (d, J = 8.5 Hz, 1H), 7.77 (d, J = 8.4 Hz, 1H), 7.63 (t, J = 7.9 Hz, 1H), 7.36 (t, J = 7.7 Hz, 1H), 7.26 (s, 4H), 5.52 (s, 2H), 3.49 (m, 2H), 3.33-3.25 (m, 2H), 2.98 (m, 4H), 2.05-1.91 (m, 2H), 1.90-1.76 (m, 2H). | 388.1 |
| 116 | 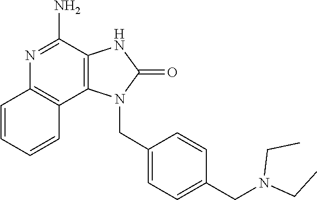 [SMILES: CCN(CC)Cc1ccc(Cn2c(=O)[nH]c3c(N)nc4ccccc4c32)cc1] | ¹H NMR (400 MHz, DMSO-d₆): δ 14.06 (s, 1H), 12.37 (s, 1H), 10.23 (s, 1H), 8.59 (s, 2H), 7.86 (d, J = 8.2 Hz, 1H), 7.77 (d, J = 8.4 Hz, 1H), 7.63-7.60 (m, 3H), 7.37-7.34 (m, 3H), 5.57 (s, 2H), 4.23 (d, J = 5.2 Hz, 2H), 2.99 (q, J = 7.2 Hz, 4H), 1.19 (t, J = 7.2 Hz, 6H). | 376.1 |
| 117 | 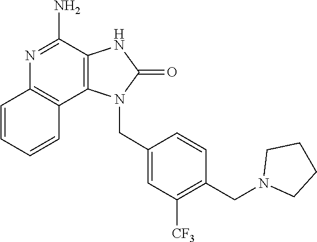 [SMILES: Nc1nc2ccccc2c2c1[nH]c(=O)n2Cc1ccc(CN2CCCC2)c(C(F)(F)F)c1] | ¹H NMR (400 MHz, DMSO-d₆): δ 11.05 (s, 1H), 7.75 (d, J = 8.0 Hz, 1H), 7.68 (d, J = 8.3 Hz, 2H), 7.53 (d, J = 8.0 Hz, 1H), 7.43 (d, J = 8.2 Hz, 1H), 7.33 (t, J = 7.2 Hz, 1H), 7.06 (t, J = 7.2 Hz, 1H), 6.38 (s, 2H), 5.53 (s, 2H), 3.67 (s, 2H), 2.41 (s, 4H), 1.67 (s, 4H). | 442.2 |
| 118 | 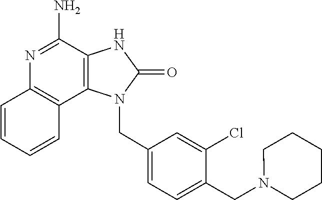 [SMILES: Nc1nc2ccccc2c2c1[nH]c(=O)n2Cc1ccc(CN2CCCCC2)c(Cl)c1] | ¹H NMR (400 MHz, MeOD) δ 7.96 (d, J = 8.2 Hz, 1H), 7.76 (d, J = 7.8 Hz, 1H), 7.71-7.67 (m, 2H), 7.59 (d, J = 1.3 Hz, 1H), 7.45-7.38 (m, 2H), 5.68 (s, 2H), 4.44 (s, 2H), 3.51-3.42 (m, 2H), 3.15-3.04 (m, 2H), 1.96- 1.87 (m, 2H), 1.83-1.70 (m, 3H), 1.58-1.46 (m, 1H). | 422.2 |
| 119 | 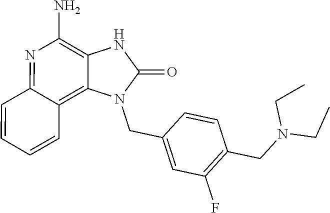 [SMILES: CCN(CC)Cc1ccc(Cn2c(=O)[nH]c3c(N)nc4ccccc4c32)cc1F] | ¹HNMR (400 MHz, DMSO-d6) δ 14.06 (s, 1H), 12.34 (s, 1H), 10.06 (s, 1H), 8.58 (s, 2H), 7.85 (d, J = 8.2 Hz, 1H), 7.78 (d, J = 8.4 Hz, 1H), 7.69 (t, J = 7.8 Hz, 1H), 7.63 (t, J = 7.7 Hz, 1H), 7.34 (dd, J = 15.5, 8.7 Hz, 2H), 7.21 (d, J = 8.1 Hz, 1H), 5.59 (s, 2H), 4.28 (d, J = 4.5 Hz, 2H), 3.05 (s, 4H), 1.22 (t, J = 7.2 Hz, 6H). | 392.2 |
| 120 | 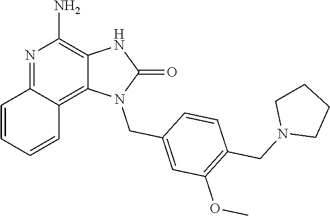 [SMILES: COc1cc(Cn2c(=O)[nH]c3c(N)nc4ccccc4c32)ccc1CN1CCCC1] | ¹H NMR (400 MHz, DMSO-d6) δ 10.99 (s, 1H), 7.77 (d, J = 8.1 Hz, 1H), 7.52 (d, J = 8.2 Hz, 1H), 7.32 (t, J = 7.5 Hz, 1H), 7.18 (d, J = 7.6 Hz, 1H), 7.07 (t, J = 7.5 Hz, 1H), 7.01 (s, 1H), 6.62 (d, J = 8.4 Hz, 1H), 6.34 (s, 2H), 5.42 (s, 3H), 3.73 (s, 2H), 3.48 (s, 2H), 2.38 (m, 4H), 1.63 (m, 4H). | 404.2 |
| 121 | 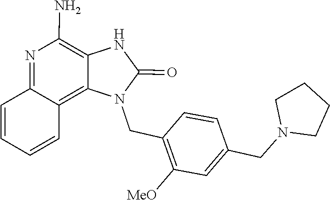 [SMILES: COc1cc(CN2CCCC2)ccc1Cn1c(=O)[nH]c2c(N)nc3ccccc3c21] | ¹H NMR (400 MHz, MeOD) δ 7.86 (d, J = 8.3 Hz, 1H), 7.74 (d, J = 8.3 Hz, 1H), 7.67 (t, J = 7.7 Hz, 1H), 7.38 (t, J = 7.6 Hz, 1H), 7.33 (s, 1H), 7.00 (s, 2H), 5.58 (s, 2H), 4.34 (s, 2H), 4.05 (s, 3H), 3.46 (s, 2H), 3.17 (s, 2H), 2.16 (s, 2H), 2.05-1.95 (m, 2H). | 404.2 |
| 122 | 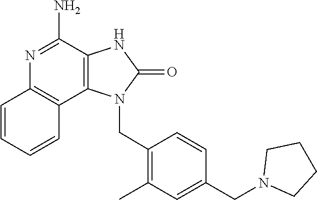 [SMILES: Cc1cc(CN2CCCC2)ccc1Cn1c(=O)[nH]c2c(N)nc3ccccc3c21] | ¹H NMR (400 MHz, MeOD) δ 7.78 (d, J = 8.2 Hz, 1H), 7.72 - 7.64 (m, 2H), 7.53 (s, 1H), 7.35 (t, J = 7.7 Hz, 1H), 7.24 (d, J = 7.5 Hz, 1H), 6.91 (d, J = 7.9 Hz, 1H), 5.63 (s, 2H), 4.33 (s, 2H), 3.46 (s, 2H), 3.15 (s, 2H), 2.62 (s, 3H), 2.17 (s, 2H), 2.01 (s, 2H). | 388.2 |
| 123 | 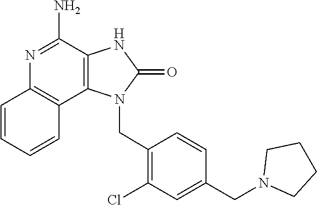 [SMILES: Nc1nc2ccccc2c2c1[nH]c(=O)n2Cc1ccc(CN2CCCC2)cc1Cl] | ¹H NMR (400 MHz, MeOD) δ 7.83 (d, J = 1.7 Hz, 1H), 7.80-7.76 (m, 1H), 7.74-7.67 (m, 2H), 7.45-7.36 (m, 2H), 7.13 (d, J = 8.0 Hz, 1H), 5.73 (s, 2H), 4.39 (s, 2H), 3.52-3.46 (m, 2H), 3.21-3.14 (m, 2H), 2.19 (s, 2H), 2.01 (d, J = 7.9 Hz, 2H). | 408.2 |
| 124 | 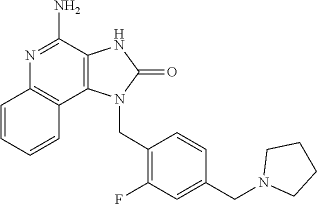 [SMILES: Nc1nc2ccccc2c2c1[nH]c(=O)n2Cc1ccc(CN2CCCC2)cc1F] | ¹H NMR (400 MHz, MeOD) δ 7.95 (d, J = 8.3 Hz, 1H), 7.75 (d, J = 22.2 Hz, 2H), 7.56-7.41 (m, 2H), 7.28 (d, J = 22.4 Hz, 2H), 5.74 (s, 2H), 4.39 (s, 2H), 3.50 (s, 2H), 3.19 (s, 2H), 2.18 (s, 2H), 2.07-1.91 (m, 2H). | 392.3 |
| 125 | 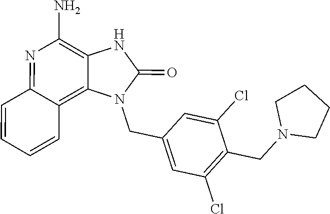 [SMILES: Nc1nc2ccccc2c2c1[nH]c(=O)n2Cc1cc(Cl)c(CN2CCCC2)c(Cl)c1] | ¹H NMR (400 MHz, MeOD) δ 7.95 (d, J = 8.1 Hz, 1H), 7.78 (d, J = 8.1 Hz, 1H), 7.72 (d, J = 7.0 Hz, 1H), 7.58 (s, 2H), 7.46 (s, 1H), 5.68 (s, 2H), 4.73 (s, 2H), 3.64 (s, 2H), 3.40 (s, 2H), 2.22 (s, 2H), 2.04 (s, 2H). | 442.1 |
| 126 | 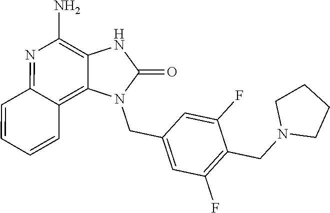 [SMILES: Nc1nc2ccccc2c2c1[nH]c(=O)n2Cc1cc(F)c(CN2CCCC2)c(F)c1] | ¹H NMR (400 MHz, DMSO-d6) δ 14.20 (s, 1H), 12.48 (s, 1H), 10.66 (s, 1H), 8.72 (s, 2H), 7.81 (dd, J = 13.0, 8.3 Hz, 2H), 7.64 (t, J = 7.8 Hz, 1H), 7.38 (t, J = 7.8 Hz, 1H), 7.23 (d, J = 8.4 Hz, 2H), 5.59 (s, 2H), 4.37 (s, 2H), 3.44 (s, 2H), 3.07 (s, 2H), 2.04- 1.81 (m, 4H). | 410.2 |

### Example 127: Preparation of 4-amino-1-((4-methyl-5-(pyrrolidin-1-ylmethyl)thien-2-yl)methyl)-1,3-dihydro-2H-imidazo[4,5-c]quinolin-2-one

The compound of Example 127 (2 mg, yield: 7%) was prepared by referring to the preparation method of Example 26. LC-MS (ESI) [M+H]⁺=394.2; ¹H NMR (400 MHz, DMSO-d₆) δ 10.94 (s, 1H), 7.97 (d, J=7.7 Hz, 1H), 7.54 (d, J=8.6 Hz, 1H), 7.36 (t, J=7.4 Hz, 1H), 7.16 (t, J=7.3 Hz, 1H), 6.74 (s, 1H), 6.33 (s, 2H), 5.48 (s, 2H), 3.54 (s, 2H), 2.42-2.36 (m, 4H), 2.02 (s, 3H), 1.77-1.54 (m, 4H).

### Example 128: Preparation of 4-amino-1-(1-(3-methyl-4-(pyrrolidin-1-ylmethyl)phenyl)ethyl)-1,3-dihydro-2H-imidazo[4,5-c]quinolin-2-one

The compound of Example 128 (0.09 g, yield: 48%) was prepared by referring to the preparation method of Example 10. LC-MS (ESI) [M+H]⁺=402.1; ¹H NMR (400 MHz, DMSO-d₆): δ 14.13 (s, 1H), 12.42 (s, 1H), 10.52 (s, 1H), 8.67 (s, 2H), 7.77 (d, J=8.4 Hz, 1H), 7.65-7.14 (m, 6H), 6.23-6.21 (m, 1H), 4.34 (d, J=5.8 Hz, 2H), 3.34 (s, 2H), 3.05 (s, 2H), 2.40 (s, 3H), 2.00 (s, 2H), 1.95 (d, J=6.9 Hz, 3H), 1.89-1.87 (m, 2H).

### Examples 129-130

The compounds of Examples 129-130 were prepared by referring to the preparation method of Example 26

| | | | LC-MS |
|---|---|---|---|
| | | | (ESI) |
| Example | Chemical structure | ¹H NMR | [M + H]⁺ |
| 129 | 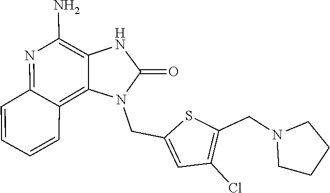 [SMILES: Nc1nc2ccccc2c2c1[nH]c(=O)n2Cc1cc(Cl)c(CN2CCCC2)s1] | ¹H NMR (400 MHz, MeOD) δ 8.23 (s, 1H), 7.79 (dd, J = 9.1, 7.7 Hz, 2H), 7.63-7.51 (m, 1H), 7.26 (s, 1H), 5.82 (s, 2H), 4.58 (s, 2H), 3.58-3.45 (m, 2H), 3.29-3.07 (m, 2H), 2.25-1.98 (m, 4H). | 414.1 |
| 130 | 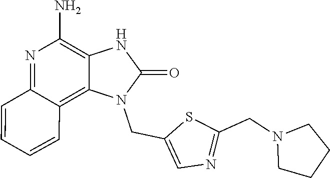 [SMILES: Nc1nc2ccccc2c2c1[nH]c(=O)n2Cc1cnc(CN2CCCC2)s1] | ¹H NMR (400 MHz, MeOD) δ 8.31 (d, J = 8.4 Hz, 1H), 8.00 (s, 1H), 7.87-7.71 (m, 2H), 7.60 (dd, J = 10.9, 4.2 Hz, 1H), 5.87 (s, 2H), 4.73 (s, 2H), 3.73-3.57 (m, 2H), 3.27-3.15 (m, 2H), 2.24 -2.08 (m, 2H), 2.06- 1.93 (m, 2H). | 381.1 |

### Example 131

The compound of Example 131 was prepared by referring to the preparation method of Example 43

| | | | LC-MS |
|---|---|---|---|
| | | | (ESI) |
| Example | Chemical structure | ¹H NMR | [M + H]⁺ |
| 131 | 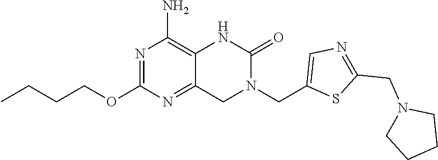 [SMILES: CCCCOc1nc(N)c2c(n1)CN(Cc1cnc(CN3CCCC3)s1)C(=O)N2] | ¹H NMR (400 MHz, DMSO) δ 8.62 (s, 1H), 7.64 (s, 1H) , 6.84 (s, 2H), 4.65 (s, 2H), 4.17 (s, 2H), 4.07 (t, J = 6.6 Hz, 2H), 3.87 (s, 2H), 3.29 (s, 1H), 2.56 (s, 4H), 1.71 (s, 4H), 1.63-1.54 (m, 2H), 1.41-1.30 (m, 2H), 0.89 (t, J = 7.4 Hz, 3H). | 418.0 |

### Examples 132-137

The compounds of Examples 132-137 were prepared by referring to the preparation method of Example 10

| | | | LC-MS |
|---|---|---|---|
| | | | (ESI) |
| Example | Chemical structure | ¹H NMR | [M + H]⁺ |
| 132 | 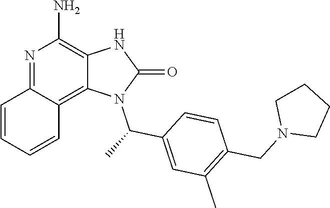 [SMILES: Cc1cc([C@H](C)n2c(=O)[nH]c3c(N)nc4ccccc4c32)ccc1CN1CCCC1] | ¹H NMR (400 MHz, DMSO-d₆): δ 14.04 (s, 1H), 12.28 (s, 1H), 10.30 (s, 1H), 8.54 (s, 2H), 7.76 (d, J = 8.4 Hz, 1H), 7.57-7.55 (m, 2H), 7.36-6.99 (m, 4H), 6.23-6.22 (m, 1H), 4.34 (d, J = 5.7 Hz, 2H), 3.33-3.23 (m, 2H), 3.06 (s, 2H), 2.39 (s, 3H), 2.01 (s, 2H), 1.95 (d, J = 6.9 Hz, 3H), 1.91-1.83 (m, 2H). | 402.2 |
| 133 | 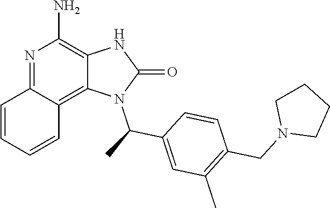 [SMILES: Cc1cc([C@@H](C)n2c(=O)[nH]c3c(N)nc4ccccc4c32)ccc1CN1CCCC1] | ¹H NMR (400 MHz, DMSO-d₆): δ 14.16 (s, 1H), 12.45 (s, 1H), 10.59 (s, 1H), 8.69 (s, 2H), 7.76- 7.75 (m, 1H), 7.61-7.59 (m, 2H), 7.32-7.25 (m, 4H), 6.23 (d, J = 6.9 Hz, 1H), 4.34 (d, J = 5.7 Hz, 2H), 3.34 (s, 2H), 3.06 (s, 2H), 2.40 (s, 3H), 2.00 (s, 2H), 1.95 (d, J = 7.1 Hz, 3H), 1.88 (d, J = 5.2 Hz, 2H). | 402.2 |
| 134 | 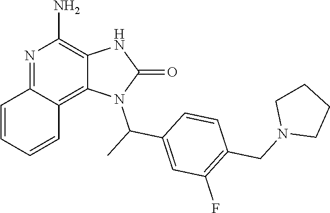 [SMILES: CC(c1ccc(CN2CCCC2)c(F)c1)n1c(=O)[nH]c2c(N)nc3ccccc3c21] | ¹H NMR (400 MHz, MeOD) δ 7.59 (d, J = 8.3 Hz, 1H), 7.43- 7.30 (m, 3H), 7.17-7.11 (m, 2H), 6.97 (s, 1H), 6.30 (q, J = 7.0 Hz, 1H), 3.69 (s, 2H), 2.55 (s, 4H), 2.03-1.99 (m, 3H), 1.77 (s, 4H). | 406.2 |
| 135 | 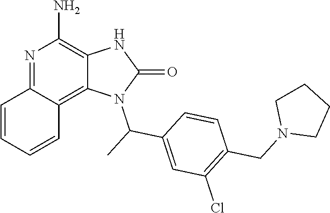 [SMILES: CC(c1ccc(CN2CCCC2)c(Cl)c1)n1c(=O)[nH]c2c(N)nc3ccccc3c21] | ¹H NMR (400 MHz, DMSO-d₆) δ 11.04 (s, 1H), 7.53 (d, J = 8.4 Hz, 1H), 7.45 (d, J = 7.6 Hz, 1H), 7.41-7.25 (m, 4H), 6.95 (s, 1H), 6.36 (s, 2H), 6.16 (d, J = 7.4 Hz, 1H), 3.65 (s, 2H), 2.45 (s, 4H), 1.91 (d, J = 6.8 Hz, 3H), 1.68 (s, 4H). | 422.2 |
| 136 | 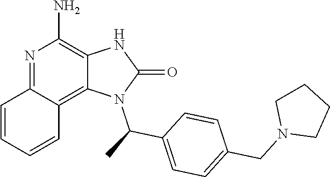 [SMILES: C[C@H](c1ccc(CN2CCCC2)cc1)n1c(=O)[nH]c2c(N)nc3ccccc3c21] | ¹H NMR (400 MHz, DMSO-d₆): δ 14.22 (s, 1H), 12.50 (s, 1H), 11.03 (s, 1H), 8.72 (s, 2H), 7.84- 7.42 (m, 7H), 7.26 (s, 1H), 6.30- 6.25 (m, 1H), 4.31 (d, J = 5.6 Hz, 2H), 3.73-3.72 (m, 2H), 3.00-2.98 (m, 2H), 1.99-1.97 (m, 5H), 1.91-1.81 (m, 2H). | 388.0 |
| 137 | 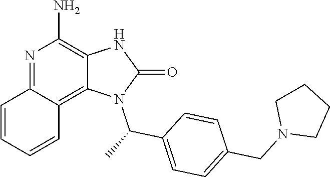 [SMILES: C[C@@H](c1ccc(CN2CCCC2)cc1)n1c(=O)[nH]c2c(N)nc3ccccc3c21] | ¹H NMR (400 MHz, DMSO-d₆): δ 14.20 (s, 1H), 12.47 (s, 1H), 10.97 (s, 1H), 8.70 (s, 2H), 7.83- 7.42 (m, 7H), 7.26 (s, 1H), 6.29- 6.27 (m, 1H), 4.32 (s, 2H), 3.27- 3.26 (m, 2H), 3.06-2.93 (m, 2H), 1.98-1.96 (m, 5H), 1.90-1.80 (m, 2H). | 388.1 |

### Examples 138-143

The compounds of Examples 138-143 were prepared by referring to the preparation method of Example 40

| | | | LC-MS |
|---|---|---|---|
| | | | (ESI) |
| Example | Chemical structure | ¹H NMR | [M + H]⁺ |
| 138 | 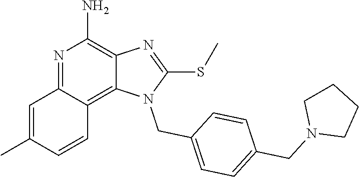 [SMILES: CSc1nc2c(N)nc3cc(C)ccc3c2n1Cc1ccc(CN2CCCC2)cc1] | ¹H NMR (400 MHz, MeOD) δ 7.71 (d, J = 8.4 Hz, 1H), 7.46 (s, 1H), 7.33 (d, J = 8.2 Hz, 2H), 7.10 (d, J = 8.1 Hz, 2H), 6.97-6.94 (m, 1H), 5.80 (s, 2H), 3.62 (s, 2H), 2.80 (s, 3H), 2.55-2.52 (m, 4H), 2.41 (s, 3H), 1.82-1.77 (m, 4H). | 418.2 |
| 139 | 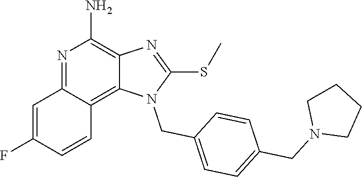 [SMILES: CSc1nc2c(N)nc3cc(F)ccc3c2n1Cc1ccc(CN2CCCC2)cc1] | ¹H NMR (400 MHz, MeOD) δ 8.02 (dd, J = 9.2, 5.4 Hz, 1H), 7.59-7.50 (m, 3H), 7.31 (d, J = 8.2 Hz, 2H), 7.22 (td, J = 8.8, 2.5 Hz, 1H), 5.93 (s, 2H), 4.38 (s, 2H), 3.47 (d, J = 8.0 Hz, 2H), 3.22-3.14 (m, 2H), 2.90 (s, 3H), 2.17 (t, J = 7.3 Hz, 2H), 2.01 (t, J = 8.1 Hz, 2H) | 422.2 |
| 140 | 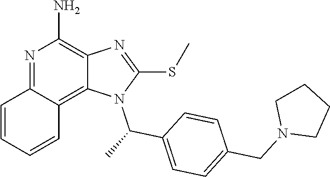 [SMILES: CSc1nc2c(N)nc3ccccc3c2n1[C@@H](C)c1ccc(CN2CCCC2)cc1] | ¹H NMR (400 MHz, MeOD) δ 7.78-7.74 (m, 1H), 7.67-7.22 (m, 7H), 6.47 (s, 1H), 4.40 (s, 2H), 3.48-3.39 (m, 2H), 3.21-3.10 (m, 2H), 2.96-2.75 (m, 3H), 2.19-2.11 (m, 5H), 2.06-1.97 (m, 2H). | 418.2 |
| 141 | 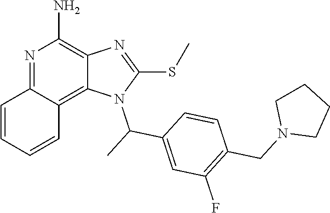 [SMILES: CSc1nc2c(N)nc3ccccc3c2n1C(C)c1ccc(CN2CCCC2)c(F)c1] | ¹H NMR (400 MHz, MeOD) δ 7.79-7.74 (m, 1H), 7.69-7.20 (m, 6H), 6.50 (s, 1H), 4.47 (s, 2H), 3.53-3.47 (m, 2H), 3.22-3.14 (m, 2H), 2.91-2.78 (m, 3H), 2.19-2.12 (m, 5H), 2.05-1.98 (m, 2H). | 436.1 |
| 142 | 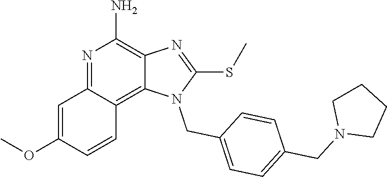 [SMILES: COc1ccc2c(c1)nc(N)c1nc(SC)n(Cc3ccc(CN4CCCC4)cc3)c12] | ¹H NMR (400 MHz, MeOD) δ 7.89 (d, J = 9.2 Hz, 1H), 7.56 (d, J = 8.1 Hz, 2H), 7.31 (d, J = 8.1 Hz, 2H), 7.24 (d, J = 2.4 Hz, 1H), 7.02 (dd, J = 9.2, 2.4 Hz, 1H), 5.90 (s, 2H), 4.37 (s, 2H), 3.93 (s, 3H), 3.47 (t, J = 9.4 Hz, 2H), 3.18 (t, J = 8.3 Hz, 2H), 2.88 (s, 3H), 2.17 (t, J = 7.0 Hz, 2H), 2.05-1.94 (m, 2H). | 434.2 |
| 143 | 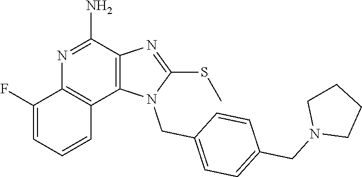 [SMILES: CSc1nc2c(N)nc3c(F)cccc3c2n1Cc1ccc(CN2CCCC2)cc1] | ¹H NMR (400 MHz, DMSO-d₆) δ 7.63 (d, J = 8.3 Hz, 1H), 7.26 - 7.16 (m, 3H), 7.06 - 7.00 (m, 3H), 6.84 (s, 2H), 5.76 (s, 2H), 3.49 (s, 2H), 2.78 (s, 3H), 2.35 (s, 4H), 1.64 (s, 4H). | 422.2 |

### Example 144: Preparation of 4-chloro-1-(4-(4-((methylamino)methyl)phenoxy)benzyl)-1,3-dihydro-2H-imidazo[4,5-c]quinolin-2-one

Step 1: The compound tert-butyl (4-(4-((4-chloro-2-oxo-2,3-dihydro-1H-imidazo[4,5-c]quinolin-1-yl)methyl)phenoxy)benzyl)(methyl)carbamate was prepared by referring to Step 1, Step 2, and Step 3 of the preparation method of Example 6.

### Step 2: Preparation of 4-chloro-1-(4-(4-((methylamino)methyl)phenoxy)benzyl)-1,3-dihydro-2H-imidazo[4,5-c]quinolin-2-one

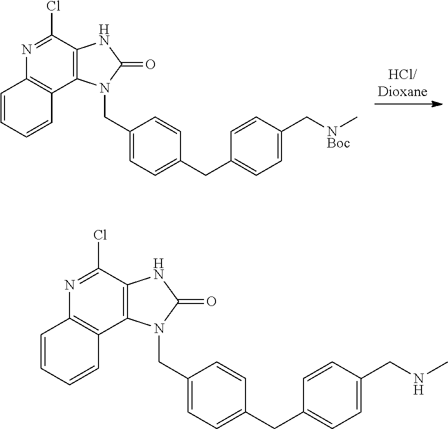 [SMILES: CN(Cc1ccc(Cc2ccc(Cn3c(=O)[nH]c4c(Cl)nc5ccccc5c43)cc2)cc1)C(=O)OC(C)(C)C ; CNCc1ccc(Cc2ccc(Cn3c(=O)[nH]c4c(Cl)nc5ccccc5c43)cc2)cc1]

Tert-butyl (4-(4-((4-chloro-2-oxo-2,3-dihydro-1H-imidazo[4,5-c]quinolin-1-yl)methyl)phenoxy)benzyl)(methyl)carbamate (50.0 mg, 91.7 μmol, 1.0 eq) was dissolved in dichloromethane (2 mL), and then a solution of HCl in dioxane (4 M, 1 mL) was added. The reaction mixture was stirred at 25° C. for 3 h. After the reaction was completed, as detected by LC-MS, the reaction mixture was concentrated to remove the solvent to give a crude product, which was separated and purified by Prep-HPLC (0.01% aqueous formic acid, MeCN) to give the target compound (23.7 mg, yield: 57.9%). LC-MS (ESI) [M+H]⁺=445.0; ¹H NMR (400 MHz, CD₃OD): δ 8.53 (s, 1H), 8.06 (d, J=8.0 Hz, 1H), 7.94 (d, J=8.0 Hz, 1H), 7.63-7.61 (m, 1H), 7.50-7.48 (m, 1H), 7.41-7.39 (m, 2H), 7.33-7.30 (m, 2H), 7.00-6.98 (m, 4H), 5.62 (s, 2H), 4.09 (s, 2H), 2.66 (s, 3H).

### Example 145: Preparation of 2-(methylthio)-1-(4-(pyrrolidin-1-ylmethyl)benzyl)-1H-imidazo[4,5-c][1,8]naphthyridine-4-amine

### Step 1: Preparation of 1,8-naphthyridine-2,4-diol

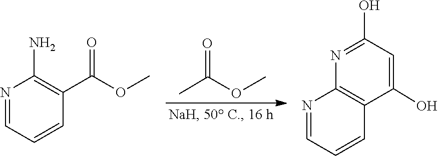

Methyl 2-aminopyridine-3-carboxylate (15.00 g, 98.59 mmol, 1 eq) was dissolved in methyl acetate (150 mL). 60% sodium hydride (9.86 g, 246.47 mmol, 2.5 eq) was added. The reaction system was stirred at 50° C. for 16 h. After the reaction was completed, as detected by LC-MS, the reaction mixture was filtered. The filter cake was dissolved in water. The aqueous solution was adjusted to pH=6 with acetic acid, and a solid precipitated. The mixture was filtered. The filter cake was dried to give the target compound (5.70 g, yield: 35.7%). LC-MS (ESI) [M+H]⁺=163.1; ¹H NMR (400 MHz, DMSO-d₆) δ 11.50 (s, 1H), 8.50 (dd, J=4.7, 1.7 Hz, 1H), 8.16 (dd, J=7.8, 1.7 Hz, 1H), 7.20 (dd, J=7.8, 4.7 Hz, 1H), 5.72 (s, 1H).

### Step 2: Preparation of 3-nitro-1,8-naphthyridine-2,4-diol

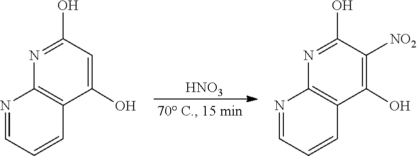 [SMILES: Oc1cc(O)c2cccnc2n1 ; "HNO3" ; O=[N+]([O-])c1c(O)nc2ncccc2c1O]

1,8-naphthyridine-2,4-diol (5.6 g, 34.54 mmol, 1 eq.) was dissolved in nitric acid (30 mL). The mixture was stirred at 25° C. for 10 min, and then stirred at 70° C. for 15 min. After the reaction was completed, as detected by LC-MS, the reaction mixture was slowly added dropwise to ice water, and a solid precipitated. The mixture was filtered. The filter cake was dried to give the target compound (4.00 g, yield: 55.9%). LC-MS (ESI) [M+H]⁺=208.1; ¹H NMR (400 MHz, DMSO-d₆) δ 12.23 (s, 1H), 8.63 (dd, J=4.6, 1.5 Hz, 1H), 8.43 (dd, J=8.0, 1.6 Hz, 1H), 7.33 (dd, J=8.0, 4.7 Hz, 1H).

### Step 3: Preparation of 2,4-di chloro-3-nitro-1,8-naphthyridine

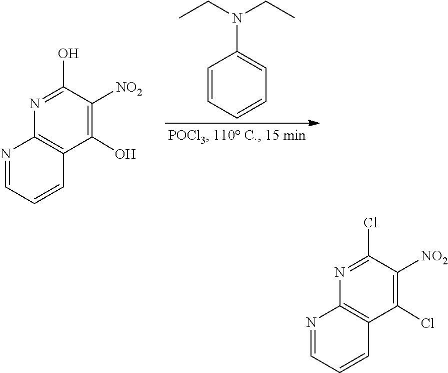

3-nitro-1,8-naphthyridine-2,4-diol (3.90 g, 18.83 mmol, 1 eq) was dissolved in phosphorus oxychloride (8660 mg, 56.48 mmol, 3 eq). N,N-dimethylaniline (4563 mg, 37.66 mmol, 2 eq) was added. The mixture was stirred at 110° C. for 15 min. After the reaction was completed, as detected by LC-MS, the reaction mixture was concentrated to remove a large amount of the phosphorus oxychloride. The reaction was quenched with water (80 mL). The mixture was extracted with ethyl acetate (50 mL×3). The organic phases were combined, washed with saturated brine (30 mL×2), dried over anhydrous sodium sulfate, filtered and concentrated to give a crude product, which was separated and purified by flash chromatography (PE:EA=100:0 to 70:30) to give the target compound (2.90 g, yield: 63.1%), LC-MS (ESI) [M+H]⁺=244.1.

### Step 4: Preparation of 2-chloro-3-nitro-N-((4-((pyrrolidin-1-yl)methyl)phenyl)methyl)-1,8-naphthyridine-4-amine

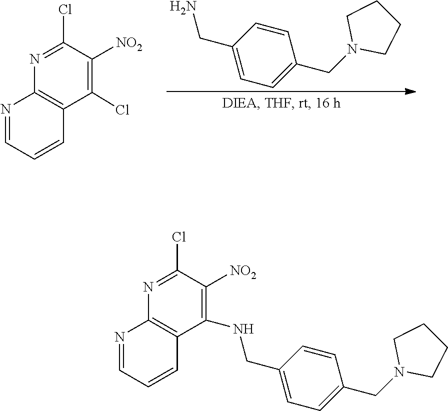 [SMILES: O=[N+]([O-])c1c(Cl)nc2ncccc2c1Cl ; NCc1ccc(CN2CCCC2)cc1 ; O=[N+]([O-])c1c(Cl)nc2ncccc2c1NCc1ccc(CN2CCCC2)cc1]

2,4-dichloro-3-nitro-1,8-naphthyridine (962 mg, 3.94 mmol, 1.5 eq) was dissolved in tetrahydrofuran (5 mL). N,N-diisopropylethylamine (1.02 g, 7.88 mmol, 3 eq) was added. A solution of 1-(4-((pyrrolidin-1-yl)methyl)phenyl)methylamine (500 mg, 2.63 mmol, 1 eq) in tetrahydrofuran (5 mL) was added dropwise to the reaction system described above. After the dropwise addition, the reaction mixture was stirred at room temperature for 16 h. After the reaction was completed, as detected by LC-MS, the reaction mixture was concentrated to remove the tetrahydrofuran to give a crude product, which was separated and purified by flash chromatography (DCM:MeOH=100:0 to 90:9) to give the target compound (500 mg, yield: 47.8%). LC-MS (ESI) [M+H]⁺=398.1; ¹H NMR (400 MHz, DMSO-d₆) δ 9.10-9.04 (m, 2H), 8.90 (s, 1H), 7.69 (dd, J=8.4, 4.4 Hz, 1H), 7.39-7.32 (m, 2H), 7.30-7.26 (m, 2H), 4.47 (s, 2H), 3.83 (s, 2H), 2.68 (s, 4H), 1.77 (s, 4H).

### Step 5: Preparation of 2-(1,3-bis(4-methoxyphenyl)propan-2 yl)-3-nitro-N-(4-(pyrrolidin-1-ylmethyl)benzyl)-1,8-naphthyridin-4-amine

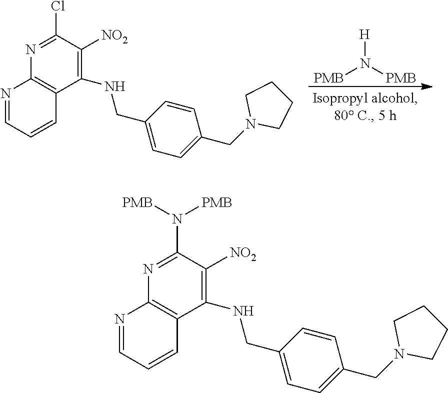

2-chloro-3-nitro-N-((4-((pyrrolidin-1-yl)methyl)phenyl)methyl)-1,8-naphthyridine-4-amine (450 mg, 1.13 mmol, 1 eq) was dissolved in isopropanol (5 mL). Bis(4-methoxybenzyl)amine (582 mg, 2.26 mmol, 2 eq) was added. The reaction mixture was stirred at 80° C. for 5 h. After the reaction was completed, as detected by LC-MS, the reaction mixture was concentrated to remove the isopropanol to give a crude product, which was purified by a reversed-phase system (aqueous trifluoroacetic acid solution:ACN=100:0 to 60:40) to give the target compound (130 mg, yield: 18.6%). LC-MS (ESI) [M+H]⁺=619.2; ¹H NMR (400 MHz, DMSO-d₆) δ 8.97 (s, 1H), 8.77-8.73 (m, 1H), 8.67-8.62 (m, 1H), 7.25-7.21 (m, 1H), 7.20-7.14 (m, 4H), 7.06-7.02 (m, 4H), 6.84-6.80 (m, 4H), 4.59 (s, 2H), 4.35 (s, 4H), 3.68 (s, 6H), 3.39 (s, 2H), 2.36 (s, 4H), 1.65 (s, 4H).

### Step 6: Preparation of N,N-bis((4-methoxyphenyl)methyl)-N-((4-((pyrrolidin-1-yl)methyl)phenyl)methyl)-1,8-naphthyridine-2,3,4-triamine

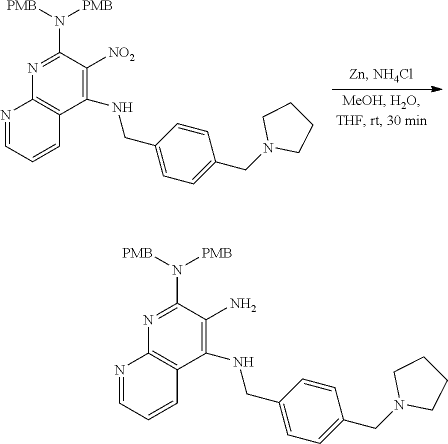

2-(1,3-bis(4-methoxyphenyl)propan-2 yl)-3-nitro-N-(4-(pyrrolidin-1 ylmethyl)benzyl)-1,8-naphthyridin-4-amine (100 mg, 0.16 mmol, 1 eq) was dissolved in methanol (1 mL), water (0.5 mL), and tetrahydrofuran (1 mL). Ammonium chloride (86 mg, 1.62 mmol, 10 eq) and zinc powder (53 mg, 0.81 mmol, 5 eq) were added sequentially. The reaction system was stirred at room temperature for 30 min. After the reaction was completed, as detected by LC-MS, the reaction mixture was filtered. The filtrate was extracted with ethyl acetate (20 mL×3). The organic phases were combined, washed with saturated brine (20 mL×2), dried over anhydrous sodium sulfate, filtered and concentrated to give a crude product of the target compound (90 mg), which was used directly in the next step. LC-MS (ESI) [M+H]⁺=589.4.

### Step 7: Preparation of 4-(bis((4-methoxyphenyl)methyl)amino)-1-((4-((pyrrolidin-1-yl)methyl)phenyl)methyl)-1,3-dihydro-2H-imidazo[4,5-c][1,8]-naphthyridine-2-thione

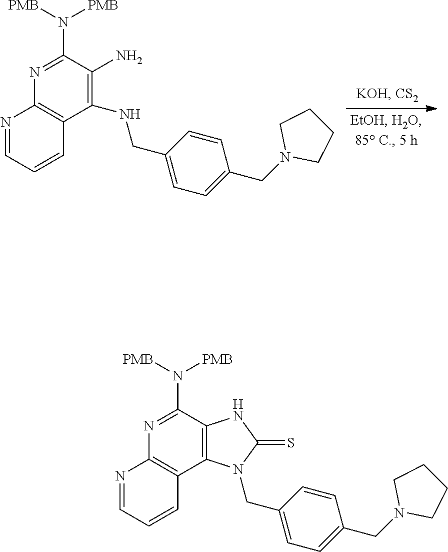

N,N-bis((4-methoxyphenyl)methyl)-N-((4-((pyrrolidin-1 yl)methyl)phenyl)methyl)-1,8-naphthyridine-2,3,4-triamine (80 mg, 0.14 mmol, 1 eq) was dissolved in a mixed solution of ethanol (2 mL) and water (0.2 mL). Potassium hydroxide (38 mg, 0.68 mmol, 5 eq) and carbon disulfide (52 mg, 0.68 mmol, 5 eq) were added sequentially under nitrogen atmosphere. The reaction system described above was stirred at 85° C. for 6 h. After the reaction was completed, as detected by LC-MS, water (10 mL) was added to the reaction mixture. The mixture was extracted with ethyl acetate (20 mL×3). The organic phases were combined, dried over anhydrous sodium sulfate, filtered and concentrated to give a crude product of the target compound (75 mg), which was used directly in the next step. LC-MS (ESI) [M+H]⁺=631.3.

### Step 8: Preparation of N,N-bis(4-methoxybenzyl)-2-(methylthio)-1-(4-(pyrrolidin-1-ylmethyl)benzyl)-1H-imidazo[4,5-c][1,8]naphthyridin-4-amine

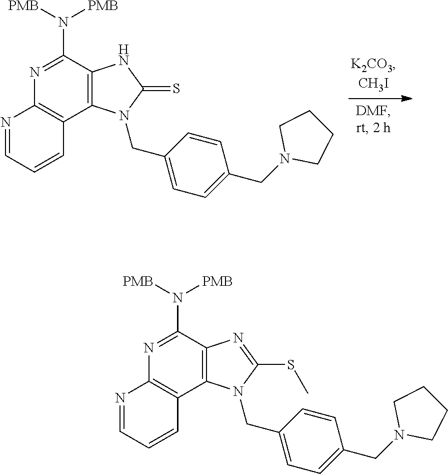

4-(bis((4-methoxyphenyl)methyl)amino)-1-((4-((pyrrolidin-1-yl)methyl)phenyl)methyl)-1,3-dihydro-2H-imidazo[4,5-c][1,8]-naphthyridine-2-thione (65 mg, 0.1 mmol, 1 eq.) was dissolved in N,N-dimethylformamide (1 mL), and potassium carbonate (21 mg, 0.15 mmol, 1.5 eq) and iodomethane (13 mg, 0.09 mmol, 0.9 eq) were added sequentially. The reaction system was stirred at 25° C. for 2 h. After the reaction was completed, as detected by LC-MS, water (10 mL) was added to the reaction mixture. The resulting mixture was extracted with ethyl acetate (20 mL×3). The organic phases were combined, dried over anhydrous sodium sulfate, filtered and concentrated to give a crude product, which was purified by silica gel column chromatography (DCM:MeOH=100:0 to 80:20) to give a crude product of the target compound (55 mg), which was used directly in the next step. LC-MS (ESI) [M+H]⁺=645.4.

### Step 9: Preparation of 2-(methylthio)-1-(4-(pyrrolidin-1-ylmethyl)benzyl)-1H-imidazo[4,5-c][1,8]naphthyridine-4-amine

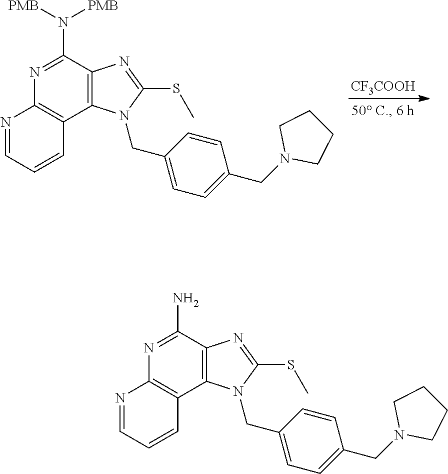

N,N-bis(4-methoxybenzyl)-2-(methylthio)-1-(4-(pyrrolidin-1 ylmethyl)benzyl)-1H-imidazo[4,5-c][1,8]naphthyridin-4-amine (45 mg, 0.07 mmol, 1 eq) was dissolved in trifluoroacetic acid (2 mL). The reaction mixture was stirred at 50° C. for 6 h. After the reaction was completed, as detected by LC-MS, the reaction mixture was concentrated to remove the trifluoroacetic acid to give a crude product, which was purified by prep-HPLC (aqueous ammonia/MeCN) to give the target compound (3.28 mg, yield: 10.6%). LC-MS (ESI) [M+H]⁺=405.2; ¹H NMR (400 MHz, MeOD) S 8.55-8.48 (m, 1H), 8.19-8.14 (m, 1H), 7.34-7.28 (m, 2H), 7.11-7.03 (m, 3H), 5.79 (s, 2H), 3.58 (s, 2H), 2.82 (s, 3H), 2.49 (s, 4H), 1.80-1.72 (m, 4H).

### Example 146: Preparation of 2-[4-amino-1-({4-[(pyrrolidin-1-yl)methyl]phenyl}methyl)-1H-imidazo[4,5-c]quinolin-2-yl]acetic acid

### Step 1: Preparation of methyl 2-(4-{bis[(4-methoxyphenyl)methyl]amino}-1-({4-[(pyrrolidin-1-yl)methyl]phenyl}methyl)-1H-imidazo[4,5-c]quinolin-2-yl)acetate

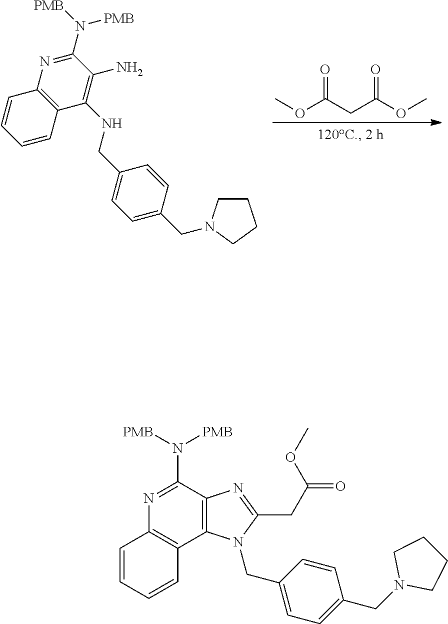

To N²,N²-bis(4-methoxybenzyl)-N⁴-(4-(pyrrolidin-1-ylmethyl)benzyl)quinoline-2,3,4-triamine (300 mg, 0.51 mmol, 1 eq.) was added dimethyl 1,3-malonate (5 mL, 37.85 mmol, 74.41 eq.). The mixture was reacted at 120° C. for 2 h. After the reaction was completed, as detected by LC-MS, the reaction mixture was subjected to column chromatography (dichloromethane:methanol=10:1) to give the target compound (273 mg, yield: 80.13%), LC-MS (ESI) [M+H]⁺=670.2.

### Step 2: Preparation of methyl 2-[4-amino-1-({4-[(pyrrolidin-1-yl)methyl]phenyl}methyl)-1H-imidazo[4,5-c]quinolin-2-yl]acetate

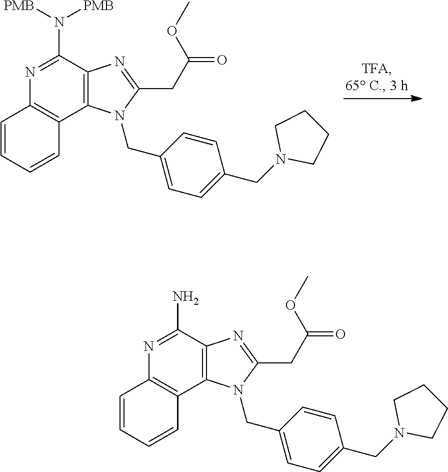

Methyl 2-(4-{bis[(4-methoxyphenyl)methyl]amino}-1-({4-[(pyrrolidin-1-yl)methyl]phenyl}methyl)-1H-imidazo[4,5-c]quinolin-2-yl)acetate (220 mg, 0.33 mmol, 1 eq.) was dissolved in trifluoroacetic acid (4 mL). The mixture was stirred at 65° C. for 3 h. After the reaction was completed, as detected by LC-MS, the reaction mixture was concentrated by rotary evaporation to give a crude product of the target compound (250 mg), which was used directly in the next step. LC-MS (ESI) [M+H]⁺=430.2.

### Step 3: Preparation of 2-[4-amino-1-({4-[(pyrrolidin-1-yl)methyl]phenyl}methyl)-1H-imidazo[4,5-c]quinolin-2-yl]acetic acid

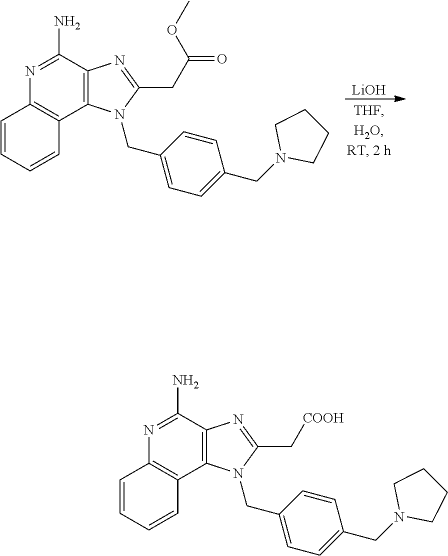

Methyl 2-[4-amino-1-({4-[(pyrrolidin-1-yl)methyl]phenyl}methyl)-1H-imidazo[4,5-c]quinolin-2-yl]acetate (200 mg, 0.47 mmol, 1 eq.) was dissolved in tetrahydrofuran (6 mL) and water (4 mL). Lithium hydroxide (33.46 mg, 1.4 mmol, 3 eq.) was added. The reaction mixture was stirred at 25° C. for 2 h. After the reaction was completed, as detected by LC-MS, the reaction mixture was concentrated by rotary evaporation. Water (15 mL) and ethyl acetate (15 mL×3) were added for extraction. The organic phase was collected and concentrated to give a crude product, which was purified by prep-HPLC (C18, 0.05% aqueous ammonia/MeCN) to give the target compound (65.74 mg, yield: 33.66%). LC-MS (ESI) [M+H]⁺=416.1; ¹H NMR (400 MHz, MeOD) δ 7.82 (d, J=8.3 Hz, 1H), 7.65 (d, J=7.9 Hz, 1H), 7.39 (s, 1H), 7.33 (d, J=8.0 Hz, 2H), 7.11 (d, J=7.4 Hz, 3H), 5.92 (s, 2H), 3.82-3.80 (m, 4H), 2.69 (s, 4H), 1.83 (s, 4H).

### Example 147: Preparation of 4-amino-1-(4-(pyrrolidin-1-ylmethyl)benzyl)-1H-imidazo[4,5-c]quinoline-2-carboxylic acid

### Step 1: Preparation of methyl 4-(bis(4-methoxybenzyl)amino)-1-(4-(pyrrolidin-1-ylmethyl)benzyl)-1H-imidazo[4,5-c]quinoline-2-carboxylate

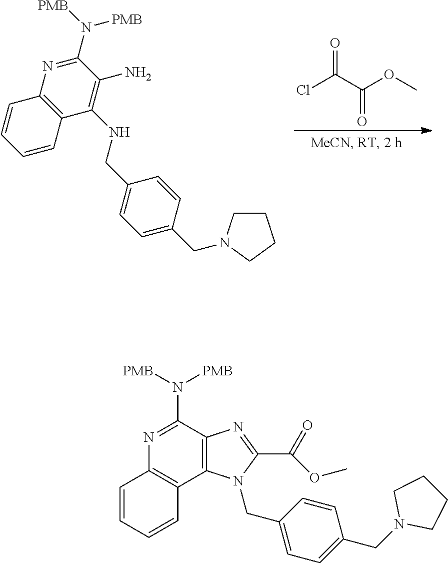

N²,N²-bis(4-methoxybenzyl)-N⁴-(4-(pyrrolidin-1-ylmethyl)benzyl)quinoline-2, 3, 4-triamine (300 mg, 0.51 mmol, 1 eq.) was dissolved in acetonitrile (6 mL). Methyl 2-chloro-2-oxoacetate (93.8 mg, 0.77 mmol, 1.5 eq.) was added. The mixture was stirred at 25° C. for 2 h. The product was detected by LC-MS and concentrated by rotary evaporation to give a crude target product (300 mg), which was used directly in the next step. LC-MS (ESI) [M+H]⁺=656.3.

### Step 2: Preparation of 4-(bis(4-methoxybenzyl)amino)-1-(4-(pyrrolidin-1-ylmethyl)benzyl)-1H-imidazo[4,5-c]quinoline-2-carboxylic acid

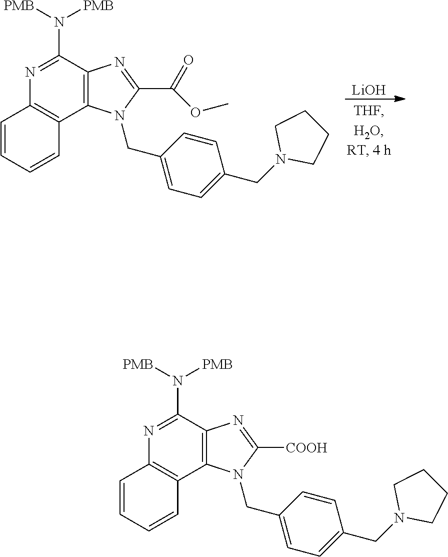

Methyl 4-(bis(4-methoxybenzyl)amino)-1-(4-(pyrrolidin-1-ylmethyl)benzyl)-1H-imidazo[4,5-c]quinoline-2-carboxylate (250 mg, 0.38 mmol, 1 eq) was dissolved in tetrahydrofuran (3 mL) and water (3 mL). Lithium hydroxide (45.65 mg, 1.91 mmol, 5 eq) was added. The mixture was stirred at 25° C. for 4 h. After the reaction was completed, as detected by LC-MS, the reaction mixture was added with water (6 mL) for dilution and extracted with ethyl acetate (10 mL×3). The organic phase was dried and concentrated to give a crude product of the target compound (240 mg), which was used directly in the next step. LC-MS (ESI) [M+H]⁺=642.2.

### Step 3: Preparation of 4-amino-1-(4-(pyrrolidin-1-ylmethyl)benzyl)-1H-imidazo[4,5-c]quinoline-2-carboxylic acid

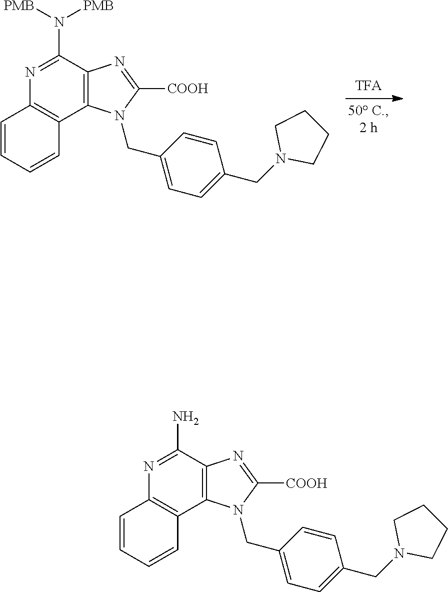

4-(bis(4-methoxybenzyl)amino)-1-(4-(pyrrolidin-1-ylmethyl)benzyl)-1H-imidazo[4,5-c]quinoline-2-carboxylic acid (50 mg, 0.08 mmol, 1 eq.) was dissolved in trifluoroacetic acid (10 mL). The mixture was stirred at 50° C. for 2 h. The product was detected by LC-MS and concentrated by rotary evaporation. The crude product was separated and purified by prep-HPLC (C18, 0.05% aqueous ammonia/MeCN) to give the target compound (13.91 mg, yield: 8.66%). LC-MS (ESI) [M+H]⁺=402.2; ¹H NMR (400 MHz, DMSO-d₆) δ 7.70 (d, J=8.7 Hz, 1H), 7.51 (d, J=7.2 Hz, 1H), 7.38-7.31 (m, 5H), 6.90 (t, J=7.3 Hz, 1H), 6.78 (s, 2H), 5.42 (s, 2H), 3.63 (s, 2H), 2.49-2.45 (m, 4H), 1.71 (s, 4H).

### Example 148: Preparation of 1-({4-[(pyrrolidin-1-yl)methyl]phenyl}methyl)-1H-imidazo[4,5-c]quinoline-4-amine

The compound of Example 148 (55.3 mg, yield: 44.2%) was prepared by referring to the preparation method of Example 2. LC-MS (ESI) [M+H]⁺=358.2; ¹H NMR (400 MHz, MeOD) δ 8.27 (s, 1H), 7.84 (dd, J=8.3, 0.9 Hz, 1H), 7.65 (d, J=7.8 Hz, 1H), 7.45-7.36 (m, 1H), 7.32 (d, J=8.1 Hz, 2H), 7.15-7.05 (m, 3H), 5.90 (s, 2H), 3.59 (s, 2H), 2.49-2.48 (m, 4H), 1.76-1.75 (m, 4H).

### Examples 149-154

The compounds of Examples 149-154 were prepared by referring to the preparation method of Example 146.

| | | | LC-MS |
|---|---|---|---|
| | | | (ESI) |
| Example | Chemical structure | ¹H NMR | [M + H]⁺ |
| 149 | 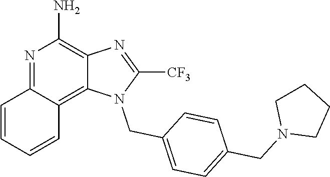 [SMILES: Nc1nc2ccccc2c2c1nc(C(F)(F)F)n2Cc1ccc(CN2CCCC2)cc1] | ¹H NMR (400 MHz, MeOD) δ 7.96-7.91 (m, 1H), 7.84-7.77 (m, 1H), 7.76-7.70 (m, 1H), 7.60-7.52 (m, 2H), 7.44-7.37 (m, 1H), 7.30- 7.24 (m, 2H), 6.17 (s, 2H), 4.36 (s, 2H), 3.49-3.41 (m, 2H), 3.19-3.11 (m, 2H), 2.20-2.10 (m, 2H), 2.04- 1.94 (m, 2H). | 426.2 |
| 150 | 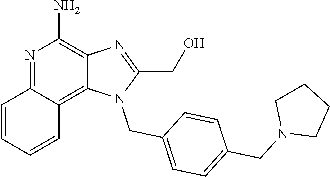 [SMILES: Nc1nc2ccccc2c2c1nc(CO)n2Cc1ccc(CN2CCCC2)cc1] | ¹H NMR (400 MHz, DMSO-d₆) δ 7.90 - 7.74 (m, 1H), 7.63 - 7.48 (m, 1H), 7.41 - 7.19 (m, 3H), 7.14 - 6.88 (m, 3H), 6.64 (s, 2H), 5.99 - 5.71 (m, 2H), 4.77 (s, 2H), 3.47 (s, 2H), 2.42 - 2.23 (m, 4H), 1.79 - 1.51 (m, 4H). | 388.2 |
| 151 | 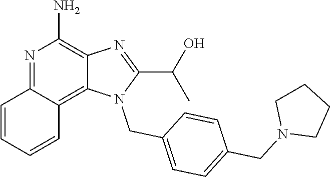 [SMILES: CC(O)c1nc2c(N)nc3ccccc3c2n1Cc1ccc(CN2CCCC2)cc1] | ¹H NMR (400 MHz, DMSO-d₆) δ 7.74 (d, J = 7.8 Hz, 1H), 7.55 (d, J = 7.5 Hz, 1H), 7.32 (t, J = 7.1 Hz, 1H), 7.21 (d, J = 8.1 Hz, 2H), 7.01 - 6.97 (m, 3H), 6.55 (s, 2H), 5.99 (q, J = 18.2 Hz, 2H), 5.09 - 5.02 (m, 1H), 3.47 (s, 3H), 2.34 (s, 4H), 1.63 (s, 4H), 1.59 (d, J = 6.5 Hz, 3H). | 402.1 |
| 152 | 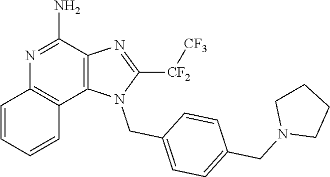 [SMILES: Nc1nc2ccccc2c2c1nc(C(F)(F)C(F)(F)F)n2Cc1ccc(CN2CCCC2)cc1] | ¹H NMR (400 MHz, MeOD) δ 7.94 (d, J = 8.3 Hz, 1H), 7.81 (d, J = 7.9 Hz, 1H), 7.73 (t, J = 7.8 Hz, 1H), 7.57 (d, J = 8.1 Hz, 2H), 7.40 (t, J = 7.8 Hz, 1H), 7.26 (d, J = 8.0 Hz, 2H), 6.22 (s, 2H), 4.36 (s, 2H), 3.45 (br.s, 2H), 3.17 (br.s, 2H), 2.15 (br.s, 2H), 1.99 (br.s, 2H). | 476.2 |
| 153 | 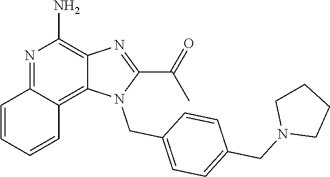 [SMILES: CC(=O)c1nc2c(N)nc3ccccc3c2n1Cc1ccc(CN2CCCC2)cc1] | ¹H NMR (400 MHz, MeOD) δ 8.03 (d, J = 8.0 Hz, 1H), 7.81 (d, J = 8.3 Hz, 1H), 7.72 (t, J = 7.5 Hz, 1H), 7.55 (d, J = 7.1 Hz, 2H), 7.41 (t, J = 7.4 Hz, 1H), 7.27 (d, J = 7.0 Hz, 2H), 6.42 (s, 2H), 4.35 (s, 2H), 3.46 - 3.45 (m, 2H), 3.16 - 3.15 (m, 2H), 2.86 (s, 3H), 2.15 - 2.14 (m, 2H), 1.99 - 1.98 (m, 2H). | 400.1 |
| 154 | 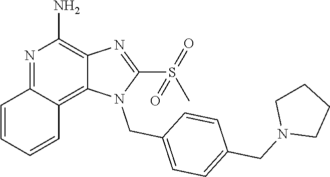 [SMILES: CS(=O)(=O)c1nc2c(N)nc3ccccc3c2n1Cc1ccc(CN2CCCC2)cc1] | ¹H NMR (400 MHz, MeOD) δ 7.97 (d, J = 8.3 Hz, 1H), 7.80 (d, J = 8.2 Hz, 1H), 7.72 (t, J = 7.8 Hz, 1H), 7.52 (d, J = 8.1 Hz, 2H), 7.39 (dd, J = 11.4, 4.2 Hz, 1H), 7.32 (d, J = 8.2 Hz, 2H), 6.39 (s, 2H), 4.34 (s, 2H), 3.67 (s, 3H), 3.45 (br.s, 2H), 3.16 (br.s, 2H), 2.15 (br.s, 2H), 1.97 (br.s, 2H). | 436.1 |

### Example 155: Preparation of 4-amino-1-(4-(pyrrolidin-1-ylmethyl)benzyl)-1,3-dihydro-2H-imidazo[4,5-c]quinoline-2-thione

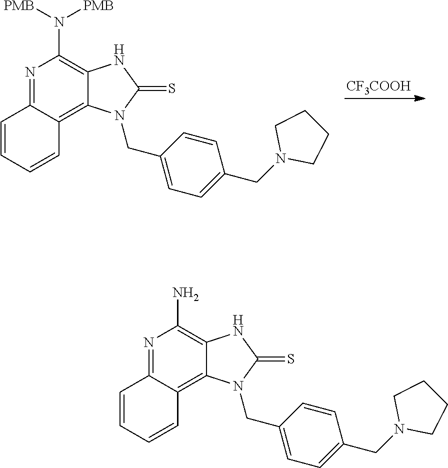

4-(bis(4-methoxybenzyl)amino)-1-(4-(pyrrolidin-1-ylmethyl)benzyl)-1,3-dihydro-2H-imidazo[4,5-c]quinoline-2-thione (150 mg, 0.24 mmol, 1 eq) was dissolved in trifluoroacetic acid (2 mL). The reaction mixture was stirred at 50° C. for 2 h. After the reaction was completed, as detected by LC-MS, the reaction mixture was concentrated to remove the trifluoroacetic acid to give a crude product, which was purified by Prep-HPLC (aqueous ammonia, MeCN) to give the target compound (15 mg, yield: 16%). LC-MS (ESI) [M+H]⁺=390.2; ¹H NMR (400 MHz, DMSO-d₆) δ 7.81 (d, J=7.9 Hz, 1H), 7.57 (d, J=7.9 Hz, 1H), 7.43-7.35 (m, 1H), 7.26 (d, J=8.0 Hz, 2H), 7.18-7.05 (m, 3H), 6.68 (s, 2H), 5.96 (s, 2H), 3.56 (s, 2H), 2.43 (s, 4H), 1.67 (s, 4H).

### Examples 156-215

The compounds of Examples 156-215 were prepared by referring to the preparation method of Example 40.

| | | | LC-MS |
|---|---|---|---|
| | | | (ESI) |
| Example | Chemical structure | ¹H NMR | [M + H]⁺ |
| 156 | 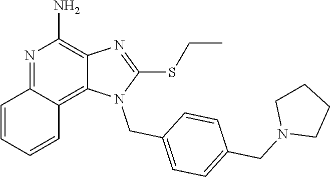 [SMILES: CCSc1nc2c(N)nc3ccccc3c2n1Cc1ccc(CN2CCCC2)cc1] | ¹H NMR (400 MHz, MeOD): δ 7.81 (d, J = 8.3 Hz, 1H), 7.64 (d, J = 8.4 Hz, 1H), 7.39 (t, J = 7.7 Hz, 1H), 7.31 (d, J = 8.1 Hz, 2H), 7.10- 7.08 (m, 3H), 5.85 (s, 2H), 3.60 (s, 2H), 3.37-3.32 (m, 2H), 2.52 (s, 4H), 1.78-1.76 (m, 4H), 1.43 (t, J = 7.3 Hz, 3H). | 418.2 |
| 157 | 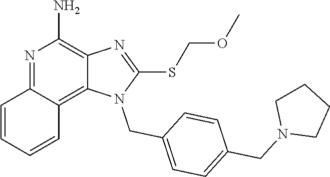 [SMILES: COCSc1nc2c(N)nc3ccccc3c2n1Cc1ccc(CN2CCCC2)cc1] | ¹H NMR (400 MHz, MeOD-d6) δ 7.81 (dd, J = 8.3, 0.9 Hz, 1H), 7.63 (d, J = 7.6 Hz, 1H), 7.40 (ddd, J = 8.4, 7.1, 1.3 Hz, 1H), 7.30 (d, J = 8.2 Hz, 2H), 7.08 (dd, J = 13.3, 4.8 Hz, 3H), 5.91 (s, 2H), 5.41 (s, 2H), 3.58 (s, 2H), 3.39 (s, 3H), 2.49 (dd, J = 9.3, 4.0 Hz, 4H), 1.77 (d, J = 3.5 Hz, 4H). | 434.2 |
| 158 | 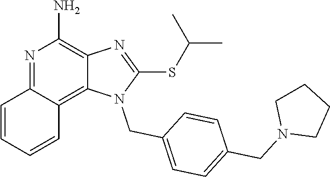 [SMILES: CC(C)Sc1nc2c(N)nc3ccccc3c2n1Cc1ccc(CN2CCCC2)cc1] | ¹H NMR (400 MHz, MeOD) δ 7.97 (d, J = 8.2 Hz, 1H), 7.77 (d, J = 8.3 Hz, 1H), 7.64 (t, J = 7.7 Hz, 1H), 7.56 (d, J = 8.1 Hz, 2H), 7.38 (t, J = 7.7 Hz, 1H), 7.26 (d, J = 8.0 Hz, 2H), 5.95 (s, 2H), 4.36 (s, 2H), 4.20 (dt, J = 13.5, 6.8 Hz, 1H), 3.45 (t, J = 10.2 Hz, 2H), 3.15 (dd, J = 18.4, 7.3 Hz, 2H), 2.15 (t, J = 7.2 Hz, 2H), 2.06-1.93 (m, 2H), 1.52 (d, J = 6.8 Hz, 6H). | 432.5 |
| 159 | 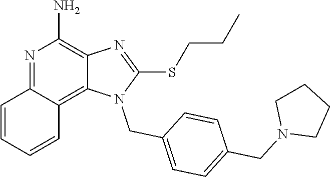 [SMILES: CCCSc1nc2c(N)nc3ccccc3c2n1Cc1ccc(CN2CCCC2)cc1] | ¹H NMR (400 MHz, DMSO-d₆) δ 14.21 (s, 1H), 11.26 (s, 1H), 7.96 (d, J = 8.1 Hz, 1H), 7.82 (d, J = 8.0 Hz, 1H), 7.63 (dd, J = 14.7, 7.8 Hz, 3H), 7.39 (t, J = 7.4 Hz, 1H), 7.21 (d, J = 8.2 Hz, 2H), 5.92 (s, 2H), 4.28 (d, J = 5.7 Hz, 2H), 3.40 (d, J = 7.1 Hz, 2H), 3.32-3.19 (m, 2H), 3.02-2.92 (m, 2H), 1.97 (t, J = 7.0 Hz, 2H), 1.91-1.81 (m, 2H), 1.77 (dd, J = 14.5, 7.2 Hz, 2H), 0.99 (t, J = 7.3 Hz, 3H). | 432.1 |
| 160 | 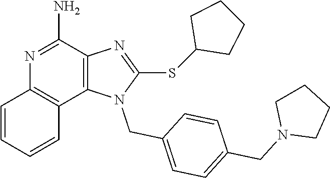 [SMILES: Nc1nc2ccccc2c2c1nc(SC1CCCC1)n2Cc1ccc(CN2CCCC2)cc1] | ¹H NMR (400 MHz, MeOD) δ 7.89 (d, J = 8.2 Hz, 1H), 7.70 (d, J = 8.1 Hz, 1H), 7.51 (dd, J = 12.3, 7.6 Hz, 3H), 7.23 (dd, J = 15.4, 7.9 Hz, 3H), 5.97 (s, 2H), 4.27 (s, 2H), 4.26-4.18 (m, 1H), 3.22 (s, 4H), 2.28 (d, J = 5.6 Hz, 2H), 2.04 (s, 4H), 1.84 (s, 2H), 1.80-1.68 (m, 4H). | 458.2 |
| 161 | 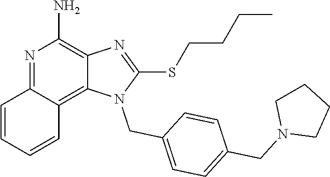 [SMILES: CCCCSc1nc2c(N)nc3ccccc3c2n1Cc1ccc(CN2CCCC2)cc1] | ¹H NMR (400 MHz, MeOD) δ 7.99 (d, J = 8.0 Hz, 1H), 7.78 (d, J = 8.4 Hz, 1H), 7.67 (t, J = 7.3 Hz, 1H), 7.57 (d, J = 8.1 Hz, 2H), 7.40 (t, J = 7.7 Hz, 1H), 7.31 (d, J = 8.1 Hz, 2H), 5.97 (s, 2H), 4.37 (s, 2H), 3.52 (d, J = 7.2 Hz, 2H), 3.46 (d, J = 9.7 Hz, 2H), 3.18 (t, J = 10.3 Hz, 2H), 2.17 (t, J = 7.1 Hz, 2H), 2.06-1.96 (m, 2H), 1.88-1.78 (m, 2H), 1.53 (dd, J = 15.0, 7.5 Hz, 2H), 0.99 (t, J = 7.4 Hz, 3H). | 446.3 |
| 162 | 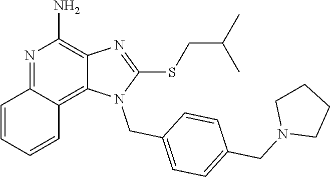 [SMILES: CC(C)CSc1nc2c(N)nc3ccccc3c2n1Cc1ccc(CN2CCCC2)cc1] | ¹H NMR (400 MHz, MeOD) δ 7.87 (d, J = 8.3 Hz, 1H), 7.66 (d, J = 8.9 Hz, 1H), 7.55 (t, J = 7.8 Hz, 1H), 7.47 (d, J = 8.2 Hz, 2H), 7.29 (t, J = 7.8 Hz, 1H), 7.19 (d, J = 8.1 Hz, 2H), 5.86 (s, 2H), 4.26 (s, 2H), 3.39-3.28 (m, 4H), 3.05 (dd, J = 18.6, 7.3 Hz, 2H), 2.05 (t, J = 9.4 Hz, 2H), 1.98 (dd, J = 13.4, 6.7 Hz, 1H), 1.94-1.83 (m, 2H), 0.98 (d, J = 6.7 Hz, 6H). | 446.3 |
| 163 | 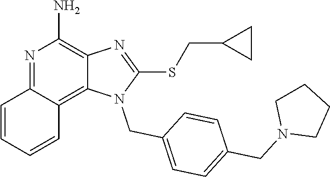 [SMILES: Nc1nc2ccccc2c2c1nc(SCC1CC1)n2Cc1ccc(CN2CCCC2)cc1] | ¹H NMR (400 MHz, MeOD) δ 8.00 (d, J = 8.4 Hz, 1H), 7.77 (d, J = 8.5 Hz, 1H), 7.67 (t, J = 7.8 Hz, 1H), 7.57 (d, J = 6.4 Hz, 2H), 7.41 (t, J = 7.8 Hz, 1H), 7.33 (d, J = 8.1 Hz, 2H), 5.98 (s, 2H), 4.37 (s, 2H), 3.46 (d, J = 7.4 Hz, 4H), 3.22-3.10 (m, 2H), 2.22-1.94 (m, 4H), 1.36-1.29 (m, 1H), 0.72-0.62 (m, 2H), 0.41 (d, J = 5.9 Hz, 2H). | 444.3 |
| 164 | 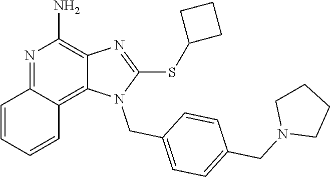 [SMILES: Nc1nc2ccccc2c2c1nc(SC1CCC1)n2Cc1ccc(CN2CCCC2)cc1] | ¹H NMR (400 MHz, MeOD) δ 8.00 (d, J = 8.4 Hz, 1H), 7.77 (d, J = 8.5 Hz, 1H), 7.67 (t, J = 7.8 Hz, 1H), 7.57 (d, J = 6.4 Hz, 2H), 7.41 (t, J = 7.8 Hz, 1H), 7.33 (d, J = 8.1 Hz, 2H), 5.98 (s, 2H), 4.37 (s, 2H), 3.46 (d, J = 7.4 Hz, 4H), 3.22-3.10 (m, 2H), 2.22-1.94 (m, 4H), 1.36-1.29 (m, 1H), 0.72-0.62 (m, 2H), 0.41 (d, J = 5.9 Hz, 2H). | 444.3 |
| 165 | 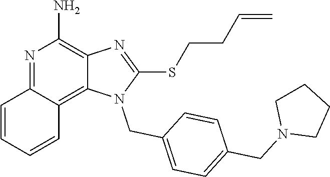 [SMILES: C=CCCSc1nc2c(N)nc3ccccc3c2n1Cc1ccc(CN2CCCC2)cc1] | ¹H NMR (400 MHz, MeOD) δ 7.97 (d, J = 8.4 Hz, 1H), 7.75 (d, J = 8.5 Hz, 1H), 7.65 (t, J = 7.9 Hz, 1H), 7.54 (d, J = 8.1 Hz, 2H), 7.38 (t, J = 8.2 Hz, 1H), 7.29 (d, J = 8.1 Hz, 2H), 5.94 (s, 2H), 5.92-5.82 (m, 1H), 5.16-5.04 (m, 2H), 4.35 (s, 2H), 3.56 (t, J = 7.1 Hz, 2H), 3.45 (s, 2H), 3.15 (d, J = 11.2 Hz, 2H), 2.60 (q, J = 7.0 Hz, 2H), 2.15 (s, 2H), 2.02-1.93 (m, 2H). | 444.2 |
| 166 | 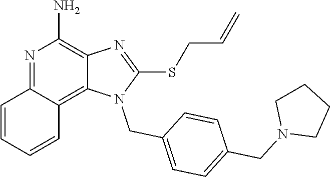 [SMILES: C=CCSc1nc2c(N)nc3ccccc3c2n1Cc1ccc(CN2CCCC2)cc1] | ¹H NMR (400 MHz, MeOD) δ 7.97 (d, J = 8.0 Hz, 1H), 7.76 (d, J = 8.2 Hz, 1H), 7.65 (t, J = 7.4 Hz, 1H), 7.55 (d, J = 8.2 Hz, 2H), 7.38 (t, J = 7.8 Hz, 1H), 7.28 (d, J = 8.1 Hz, 2H), 6.06 (ddt, J = 17.0, 10.0, 7.0 Hz, 1H), 5.95 (s, 2H), 5.40 (dd, J = 16.9, 1.2 Hz, 1H), 5.17 (d, J = 10.7 Hz, 1H), 4.35 (s, 2H), 4.12 (d, J = 7.0 Hz, 2H), 3.48-3.41 (m, 2H), 3.15 (dd, J = 18.4, 7.2 Hz, 2H), | 430.3 |
| | | 2.15 (s, 2H), 2.02-1.92 (m, 2H). | |
| 167 | 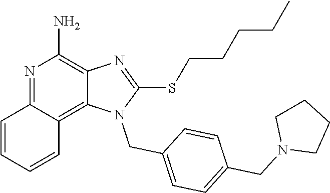 [SMILES: CCCCCSc1nc2c(N)nc3ccccc3c2n1Cc1ccc(CN2CCCC2)cc1] | ¹H NMR (400 MHz, MeOD) δ 7.97 (d, J = 7.8 Hz, 1H), 7.76 (d, J = 7.7 Hz, 1H), 7.67-7.61 (m, 1H), 7.55 (d, J = 8.2 Hz, 2H), 7.41-7.35 (m, 1H), 7.28 (d, J = 8.2 Hz, 2H), 5.95 (s, 2H), 4.35 (s, 2H), 3.49 (t, J = 7.3 Hz, 4H), 3.14 (d, J = 12.1 Hz, 2H), 2.06 (d, J = 39.8 Hz, 4H), 1.87-1.79 (m, 2H), 1.51-1.43 (m, 2H), 1.38 (dd, J = 14.8, 7.3 Hz, 2H), 0.92 (t, J = 7.2 Hz, 3H). | 460.2 |
| 168 | 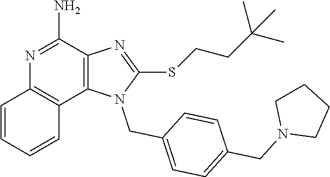 [SMILES: CC(C)(C)CCSc1nc2c(N)nc3ccccc3c2n1Cc1ccc(CN2CCCC2)cc1] | ¹H NMR (400 MHz, MeOD) δ 7.97 (d, J = 7.7 Hz, 1H), 7.76 (d, J = 7.7 Hz, 1H), 7.68-7.62 (m, 1H), 7.54 (d, J = 8.2 Hz, 2H), 7.41-7.35 (m, 1H), 7.28 (d, J = 8.2 Hz, 2H), 5.95 (s, 2H), 4.35 (s, 2H), 3.52-3.37 (m, 4H), 3.13 (d, J = 1.6 Hz, 2H), 2.07 (d, J = 55.9 Hz, 4H), 1.73-1.64 (m, 2H), 1.00 (s, 9H). | 474.3 |
| 169 | 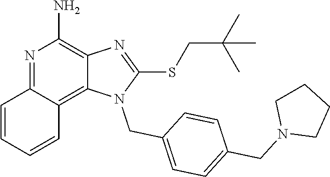 [SMILES: CC(C)(C)CSc1nc2c(N)nc3ccccc3c2n1Cc1ccc(CN2CCCC2)cc1] | ¹H NMR (400 MHz, MeOD) δ 7.83 (d, J = 8.2 Hz, 1H), 7.64 (d, J = 8.4 Hz, 1H), 7.43-7.30 (m, 3H), 7.11 (d, J = 8.2 Hz, 3H), 5.89 (s, 2H), 3.67 (s, 2H), 3.41 (s, 2H), 2.59 (s, 4H), 1.80 (d, J = 3.2 Hz, 4H), 1.05 (s, 9H). | 460.3 |
| 170 | 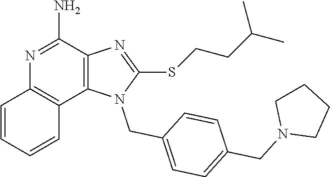 [SMILES: CC(C)CCSc1nc2c(N)nc3ccccc3c2n1Cc1ccc(CN2CCCC2)cc1] | ¹H NMR (400 MHz, MeOD) δ 7.99 (d, J = 8.0 Hz, 1H), 7.77 (d, J = 8.2 Hz, 1H), 7.68 (d, J = 7.4 Hz, 1H), 7.56 (d, J = 6.2 Hz, 2H), 7.42 (d, J = 7.8 Hz, 1H), 7.31 (d, J = 7.8 Hz, 2H), 5.97 (s, 2H), 4.37 (s, 2H), 3.54 (d, J = 7.4 Hz, 2H), 3.47 (s, 2H), 3.16 (s, 2H), 2.18 (s, 2H), 2.01 (s, 2H), 1.78 (dd, J = 13.0, 7.0 Hz, 1H), 1.76-1.69 (m, 2H), 0.99 (d, J = 6.4 Hz, 6H). | 460.2 |
| 171 | 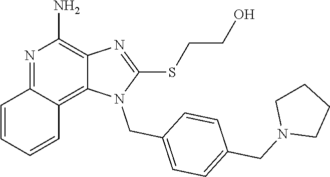 [SMILES: Nc1nc2ccccc2c2c1nc(SCCO)n2Cc1ccc(CN2CCCC2)cc1] | ¹H NMR (400 MHz, DMSO-d₆) δ 14.11 (s, 1H), 11.11 (s, 1H), 8.85 (s, 2H), 7.96 (d, J = 8.2 Hz, 1H), 7.82 (d, J = 8.3 Hz, 1H), 7.63 (dd, J = 18.9, 7.9 Hz, 3H), 7.39 (t, J = 7.7 Hz, 1H), 7.23 (d, J = 8.1 Hz, 2H), 5.93 (s, 2H), 4.28 (d, J = 5.7 Hz, 2H), 3.76 (t, J = 6.2 Hz, 2H), 3.52 (t, J = 6.2 Hz, 2H), 3.27 (d, J = 5.3 Hz, 2H), 3.03-2.93 (m, 2H), 1.96 (d, J = 5.8 Hz, 2H), 1.91-1.80 (m, 2H). | 434.2 |
| 172 | 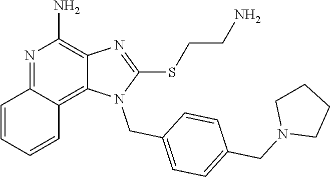 [SMILES: NCCSc1nc2c(N)nc3ccccc3c2n1Cc1ccc(CN2CCCC2)cc1] | ¹H NMR (400 MHz, MeOD) δ 7.98 (d, J = 8.2 Hz, 1H), 7.79 (d, J = 8.3 Hz, 1H), 7.67 (t, J = 7.6 Hz, 1H), 7.58 (d, J = 8.1 Hz, 2H), 7.40 (t, J = 7.8 Hz, 1H), 7.33 (d, J = 8.0 Hz, 2H), 5.96 (s, 2H), 4.36 (s, 2H), 3.76 (t, J = 6.2 Hz, 2H), 3.56 (d, J = 6.1 Hz, 2H), 3.45 (t, J = 9.6 Hz, 2H), 3.18-3.11 (m, 2H), 2.15 (t, J = 7.2 Hz, 2H), 1.99 (t, J = 8.7 Hz, 2H). | 433.2 |
| 173 | 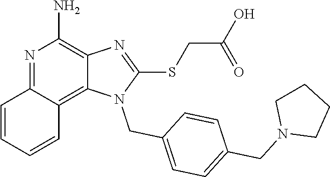 [SMILES: Nc1nc2ccccc2c2c1nc(SCC(=O)O)n2Cc1ccc(CN2CCCC2)cc1] | ¹H NMR (400 MHz, DMSO-d₆) δ 7.81 (d, J = 7.6 Hz, 1H), 7.57 (d, J = 8.1 Hz, 1H), 7.37-7.27 (m, 3H), 7.10 (dd, J = 15.9, 7.7 Hz, 3H), 6.69 (s, 2H), 5.84 (s, 2H), 4.17 (s, 2H), 3.71 (s, 2H), 2.59 (s, 4H), 1.71 (s, 4H). | 448.2 |
| 174 | 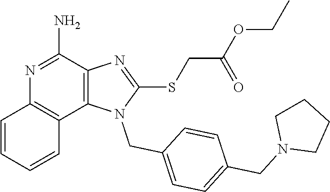 [SMILES: CCOC(=O)CSc1nc2c(N)nc3ccccc3c2n1Cc1ccc(CN2CCCC2)cc1] | ¹H NMR (400 MHz, DMSO-d₆) δ 7.83 (d, J = 8.0 Hz, 1H), 7.57 (d, J = 8.2 Hz, 1H), 7.36 (t, J = 7.5 Hz, 1H), 7.28 (d, J = 7.8 Hz, 2H), 7.14- 7.01 (m, 3H), 6.55 (s, 2H), 5.84 (s, 2H), 4.27 (s, 2H), 4.12 (q, J = 7.1 Hz, 2H), 3.57 (s, 2H), 2.44 (s, 4H), 1.67 (s, 4H), 1.17 (t, J = 7.1 Hz, 3H). | 476.3 |
| 175 | 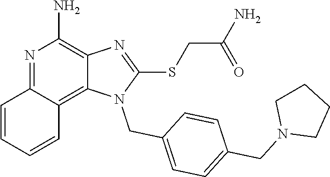 [SMILES: NC(=O)CSc1nc2c(N)nc3ccccc3c2n1Cc1ccc(CN2CCCC2)cc1] | ¹H NMR (400 MHz, MeOD) δ 7.98 (d, J = 8.3 Hz, 1H), 7.76 (d, J = 8.4 Hz, 1H), 7.65 (t, J = 7.8 Hz, 1H), 7.55 (d, J = 8.2 Hz, 2H), 7.39 (t, J = 7.8 Hz, 1H), 7.33 (d, J = 8.2 Hz, 2H), 5.96 (s, 2H), 4.35 (s, 2H), 4.16 (s, 2H), 3.50-3.40 (m, 2H), 3.20- 3.09 (m, 2H), 2.21-2.09 (m, 2H), 2.04-1.91 (m, 2H). | 447.2 |
| 176 | 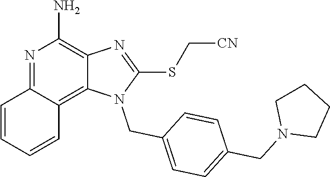 [SMILES: N#CCSc1nc2c(N)nc3ccccc3c2n1Cc1ccc(CN2CCCC2)cc1] | ¹H NMR (400 MHz, MeOD) δ 8.01 (d, J = 8.0 Hz, 1H), 7.78 (d, J = 8.3 Hz, 1H), 7.68 (t, J = 7.4 Hz, 1H), 7.56 (d, J = 8.1 Hz, 2H), 7.41 (s, 1H), 7.31 (d, J = 8.1 Hz, 2H), 5.98 (s, 2H), 4.49 (s, 2H), 4.35 (s, 2H), 3.50-3.38 (m, 2H), 3.18-3.08 (m, 2H), 2.23-2.08 (m, 2H), 2.04-1.91 (m, 2H). | 447.2 |
| 177 | 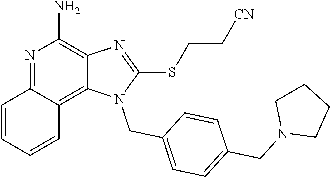 [SMILES: N#CCCSc1nc2c(N)nc3ccccc3c2n1Cc1ccc(CN2CCCC2)cc1] | ¹H NMR (400 MHz, DMSO-d6) δ 7.82 (d, J = 8.1 Hz, 1H), 7.57 (d, J = 8.3 Hz, 1H), 7.35 (t, J = 7.7 Hz, 1H), 7.25 (d, J = 8.0 Hz, 2H), 7.06 (d, J = 7.9 Hz, 3H), 6.69 (s, 2H), 5.79 (s, 2H), 3.56 (t, J = 6.7 Hz, 2H), 3.50 (s, 2H), 3.20 (t, J = 6.7 Hz, 2H), 2.36 (s, 4H), 1.64 (s, 4H). | 443.2 |
| 178 | 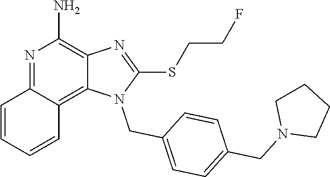 [SMILES: Nc1nc2ccccc2c2c1nc(SCCF)n2Cc1ccc(CN2CCCC2)cc1] | ¹H NMR (400 MHz, DMSO-d₆) δ 14.05 (s, 1H), 11.11 (s, 1H), 8.81 (s, 1H), 7.97 (d, J = 8.1 Hz, 1H), 7.83 (d, J = 8.3 Hz, 1H), 7.70 - 7.56 (m, 3H), 7.39 (t, J = 7.7 Hz, 1H), 7.23 (d, J = 7.7 Hz, 2H), 5.93 (s, 2H), 4.87 (t, J = 5.6 Hz, 1H), 4.75 (t, J = 5.7 Hz, 1H), 4.29 (s, 2H), 3.74 (dt, J = 11.1, 5.8 Hz, 2H), 3.27 (s, 2H), 2.99 (s, 2H), 1.91 (d, J = 48.0 Hz, 4H). | 436.3 |
| 179 | 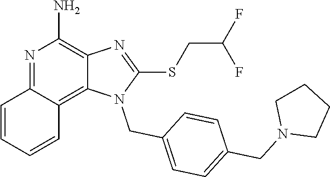 [SMILES: Nc1nc2ccccc2c2c1nc(SCC(F)F)n2Cc1ccc(CN2CCCC2)cc1] | ¹H NMR (400 MHz, DMSO-d₆) δ 7.82 (d, J = 7.8 Hz, 1H), 7.57 (d, J = 7.7 Hz, 1H), 7.35 (dd, J = 11.3, 4.1 Hz, 1H), 7.25 (d, J = 8.1 Hz, 2H), 7.06 (t, J = 7.6 Hz, 3H), 6.70 (s, 2H), 6.68-6.39 (m, 1H), 5.82 (s, 2H), 3.90-3.81 (m, 2H), 3.49 (s, 2H), 2.36 (s, 4H), 1.64 (t, J = 3.2 Hz, 4H). | 454.2 |
| 180 | 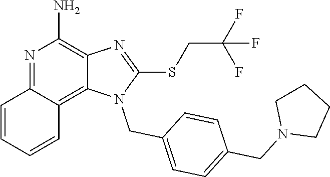 [SMILES: Nc1nc2ccccc2c2c1nc(SCC(F)(F)F)n2Cc1ccc(CN2CCCC2)cc1] | ¹H NMR (400 MHz, DMSO-d6) δ 7.83 (d, J = 8.1 Hz, 1H), 7.58 (d, J = 8.3 Hz, 1H), 7.37 (t, J = 7.7 Hz, 1H), 7.25 (d, J = 8.0 Hz, 2H), 7.06 (dd, J = 18.7, 7.9 Hz, 3H), 6.70 (s, 2H), 5.85 (s, 2H), 4.43 (q, J = 10.2 Hz, 2H), 3.49 (s, 2H), 2.36 (s, 4H), 1.64 (s, 4H). | 472.2 |
| 181 | 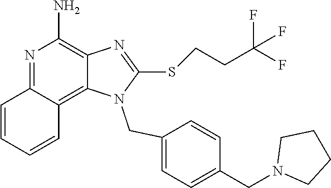 [SMILES: Nc1nc2ccccc2c2c1nc(SCCC(F)(F)F)n2Cc1ccc(CN2CCCC2)cc1] | ¹H NMR (400 MHz, MeOD) δ 7.98 (d, J = 7.7 Hz, 1H), 7.77 (d, J = 7.8 Hz, 1H), 7.69 - 7.63 (m, 1H), 7.55 (d, J = 8.2 Hz, 2H), 7.42 - 7.36 (m, 1H), 7.29 (d, J = 8.2 Hz, 2H), 5.94 (s, 2H), 4.83 - 4.81 (m, 1H), 4.35 (s, 2H), 3.68 - 3.61 (m, 2H), 3.46 (d, J = 13.2 Hz, 2H), 3.13 (d, J = 1.7 Hz, 2H), 2.91 - 2.78 (m, 2H), 2.15 (s, 2H), 1.99 (s, 2H). | 486.2 |
| 182 | 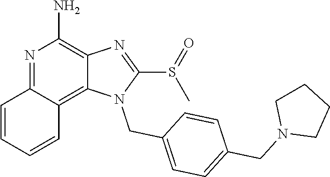 [SMILES: CS(=O)c1nc2c(N)nc3ccccc3c2n1Cc1ccc(CN2CCCC2)cc1] | ¹H NMR (400 MHz, MeOD) δ 7.85 (d, J = 7.4 Hz, 1H), 7.66 (d, J = 9.1 Hz, 1H), 7.50-7.42 (m, 1H), 7.33 (d, J = 8.2 Hz, 2H), 7.12-7.15 (m, 3H), 6.19 (dd, J = 43.1, 17.8 Hz, 2H), 3.63 (s, 2H), 3.27 (s, 3H), 2.53 (d, J = 5.2 Hz, 4H), 1.84-1.69 (m, 4H). | 420.0 |
| 183 | 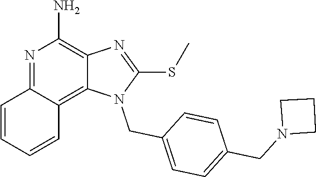 [SMILES: CSc1nc2c(N)nc3ccccc3c2n1Cc1ccc(CN2CCC2)cc1] | ¹H NMR (400 MHz, DMSO-d₆) δ 7.81 (d, J = 8.2 Hz, 1H), 7.57 (d, J = 8.2 Hz, 1H), 7.34 (t, J = 7.7 Hz, 1H), 7.23-7.18 (m, 2H), 7.08-7.02 (m, 3H), 6.58 (s, 2H), 5.76 (s, 2H), 3.45 (s, 2H), 3.07 (t, J = 6.9 Hz, 4H), 2.76 (s, 3H), 1.97-1.89 (m, 2H). | 390.2 |
| 184 | 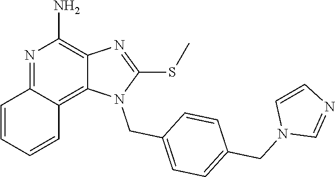 [SMILES: CSc1nc2c(N)nc3ccccc3c2n1Cc1ccc(Cn2ccnc2)cc1] | ¹H NMR (400 MHz, DMSO-d₆) δ 7.79 (d, J = 8.2 Hz, 1H), 7.69 (s, 1H), 7.57 (d, J = 8.3 Hz, 1H), 7.34 (t, J = 7.6 Hz, 1H), 7.20 (d, J = 7.7 Hz, 2H), 7.13 (s, 1H), 7.06 (dd, J = 16.8, 8.0 Hz, 3H), 6.87 (s, 1H), 6.59 (s, 2H), 5.78 (s, 2H), 5.13 (s, 2H), 2.75 (s, 3H). | 401.2 |
| 185 | 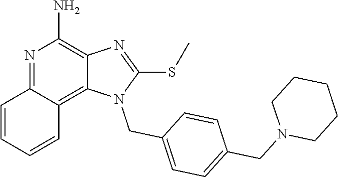 [SMILES: CSc1nc2c(N)nc3ccccc3c2n1Cc1ccc(CN2CCCCC2)cc1] | ¹H NMR (400 MHz, DMSO-d6) δ 14.28 (s, 1H), 10.95 (s, 1H), 8.87 (s, 2H), 7.97 (d, J = 8.2 Hz, 1H), 7.81 (d, J = 8.2 Hz, 1H), 7.64 (t, J = 8.6 Hz, 3H), 7.48-7.34 (m, 1H), 7.31-7.14 (m, 2H), 5.91 (s, 2H), 4.19 (d, J = 5.2 Hz, 2H), 3.18 (d, J = 11.6 Hz, 2H), 2.84 (s, 3H), 2.80- 2.67 (m, 2H), 1.86-1.60 (m, 5H), 1.44-1.18 (m, 1H). | 418.2 |
| 186 | 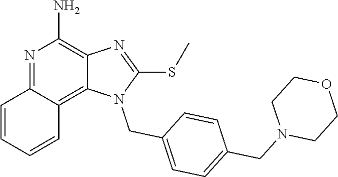 [SMILES: CSc1nc2c(N)nc3ccccc3c2n1Cc1ccc(CN2CCOCC2)cc1] | ¹H NMR (400 MHz, DMSO-d₆) δ 14.19 (s, 1H), 11.66 (s, 1H), 7.97 (d, J = 8.2 Hz, 1H), 7.81 (d, J = 8.4 Hz, 1H), 7.65 (t, J = 8.0 Hz, 3H), 7.39 (t, J = 7.7 Hz, 1H), 7.25 (d, J = 7.9 Hz, 2H), 5.91 (s, 2H), 4.27 (d, J = 4.0 Hz, 2H), 3.92 - 3.77 (m, 4H), 3.15 - 2.99 (m, 4H), 2.83 (s, 3H). | 420.9 |
| 187 | 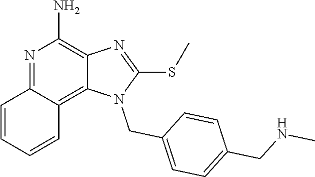 [SMILES: CNCc1ccc(Cn2c(SC)nc3c(N)nc4ccccc4c32)cc1] | ¹H NMR (400 MHz, MeOD) δ 7.97 (d, J = 8.1 Hz, 1H), 7.75 (d, J = 7.9 Hz, 1H), 7.64 (t, J = 7.8 Hz, 1H), 7.49 (d, J = 8.2 Hz, 2H), 7.37 (t, J = 7.3 Hz, 1H), 7.28 (d, J = 8.1 Hz, 2H), 5.92 (s, 2H), 4.15 (s, 2H), 2.88 (s, 3H), 2.68 (s, 3H). | 364.1 |
| 188 | 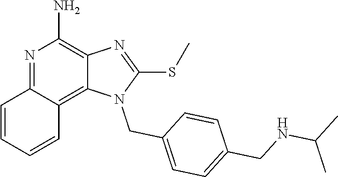 [SMILES: CSc1nc2c(N)nc3ccccc3c2n1Cc1ccc(CNC(C)C)cc1] | ¹H NMR (400 MHz, DMSO-d6) δ 14.29 (s, 1H), 9.33 (s, 2H), 8.92 (s, 2H), 7.97 (d, J = 8.3 Hz, 1H), 7.81 (d, J = 8.3 Hz, 1H), 7.70 - 7.56 (m, 3H), 7.39 (t, J = 7.7 Hz, 1H), 7.21 (d, J = 8.1 Hz, 2H), 5.90 (s, 2H), 4.15 - 4.00 (m, 2H), 3.31 - 3.13 (m, 1H), 2.84 (s, 3H), 1.27 (d, J = 6.5 Hz, 6H). | 392.1 |
| 189 | 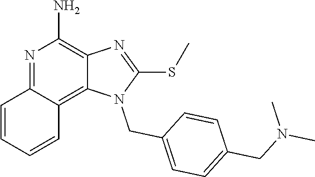 [SMILES: CSc1nc2c(N)nc3ccccc3c2n1Cc1ccc(CN(C)C)cc1] | ¹H NMR (400 MHz, DMSO-d₆) δ 14.14 (s, 1H), 10.91 (s, 1H), 8.84 (s, 2H), 7.96 (d, J = 8.0 Hz, 1H), 7.82 (d, J = 7.7 Hz, 1H), 7.64 (dd, J = 11.5, 4.2 Hz, 1H), 7.57 (d, J = 8.2 Hz, 2H), 7.38 (dd, J = 11.4, 4.1 Hz, 1H), 7.25 (d, J = 8.2 Hz, 2H), 5.91 (s, 2H), 4.22 (d, J = 5.1 Hz, 2H), 2.84 (s, 3H), 2.62 (d, J = 4.5 Hz, 6H). | 378.2 |
| 190 | 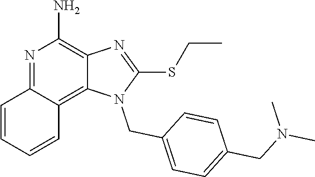 [SMILES: CCSc1nc2c(N)nc3ccccc3c2n1Cc1ccc(CN(C)C)cc1] | ¹H NMR (400 MHz, MeOD) δ 7.96 (d, J = 8.3 Hz, 1H), 7.75 (d, J = 8.2 Hz, 1H), 7.64 (s, 1H), 7.52 (d, J = 8.2 Hz, 2H), 7.37 (s, 1H), 7.30 (d, J = 8.1 Hz, 2H), 5.94 (s, 2H), 4.28 (s, 2H), 3.47 (t, J = 7.3 Hz, 2H), 2.81 (s, 6H), 1.49 (t, J = 7.3 Hz, 3H).. | 392.2 |
| 191 | 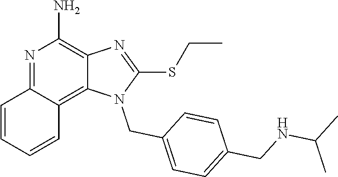 [SMILES: CCSc1nc2c(N)nc3ccccc3c2n1Cc1ccc(CNC(C)C)cc1] | ¹H NMR (400 MHz, DMSO-d₆) δ 14.27 (s, 1H), 9.29 (s, 2H), 7.96 (d, J = 8.2 Hz, 1H), 7.82 (d, J = 8.4 Hz, 1H), 7.64 (t, J = 7.8 Hz, 1H), 7.60-7.59 (m, 2H), 7.38 (t, J = 7.5 Hz, 1H), 7.19 (d, J = 8.0 Hz, 2H), 5.91 (s, 2H), 4.06 (s, 2H), 3.43-3.38 (m, 2H), 3.23-3.21 (m, 1H), 1.41 (t, J = 7.3 Hz, 3H), 1.27 (d, J = 6.5 Hz, 6H). | 406.2 |
| 192 | 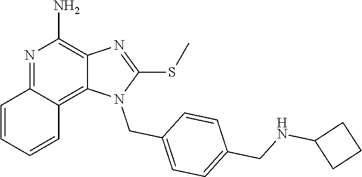 [SMILES: CSc1nc2c(N)nc3ccccc3c2n1Cc1ccc(CNC2CCC2)cc1] | ¹H NMR (400 MHz, MeOD) δ 7.96 (d, J = 8.3 Hz, 1H), 7.75 (d, J = 8.4 Hz, 1H), 7.64 (t, J = 7.8 Hz, 1H), 7.49 (d, J = 8.2 Hz, 2H), 7.36 (t, J = 7.8 Hz, 1H), 7.27 (d, J = 8.0 Hz, 2H), 5.92 (s, 2H), 4.06 (s, 2H), 3.79 - 3.70 (m, 1H), 2.88 (s, 3H), 2.27 (t, J = 8.5 Hz, 2H), 2.21 - 2.09 (m, 2H), 1.95 - 1.84 (m, 2H). | 404.1 |
| 193 | 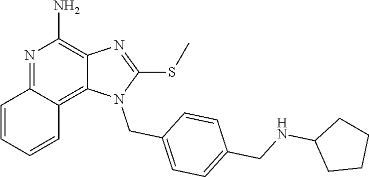 [SMILES: CSc1nc2c(N)nc3ccccc3c2n1Cc1ccc(CNC2CCCC2)cc1] | ¹H NMR (400 MHz, DMSO-d₆) δ 7.87-7.80 (m, 1H), 7.57 (d, J = 8.3 Hz, 1H), 7.37-7.32 (m, 1H), 7.27 (d, J = 8.1 Hz, 2H), 7.08-7.01 (m, 3H), 6.57 (s, 2H), 5.77 (s, 2H), 3.59 (s, 2H), 2.96-2.88 (m, 1H), 2.77 (s, 3H), 1.97 (s, 1H), 1.68-1.55 (m, 4H), 1.46-1.37 (m, 2H), 1.31-1.24 (m, 2H). | 418.2 |
| 194 | 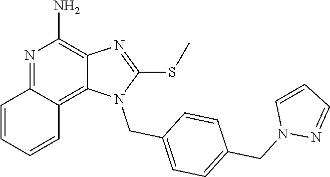 [SMILES: CSc1nc2c(N)nc3ccccc3c2n1Cc1ccc(Cn2cccn2)cc1] | ¹H NMR (400 MHz, MeOD) δ 7.97 (d, J = 8.3 Hz, 1H), 7.73 (d, J = 8.4 Hz, 1H), 7.68-7.60 (m, 2H), 7.50 (d, J = 1.8 Hz, 1H), 7.39 (dd, J = 11.4, 4.2 Hz, 1H), 7.16 (dd, J = 20.4, 8.3 Hz, 4H), 6.31 (t, J = 2.1 Hz, 1H), 5.85 (s, 2H), 5.32 (s, 2H), 2.86 (s, 3H). | 401.2 |
| 195 | 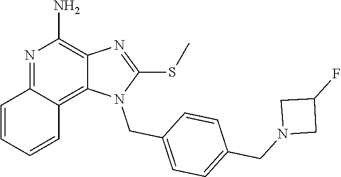 [SMILES: CSc1nc2c(N)nc3ccccc3c2n1Cc1ccc(CN2CC(F)C2)cc1] | ¹H NMR (400 MHz, DMSO-d₆) δ 7.81 (d, J = 8.1 Hz, 1H), 7.57 (d, J = 8.4 Hz, 1H), 7.35 (t, J = 7.7 Hz, 1H), 7.22 (d, J = 7.7 Hz, 2H), 7.09 - 7.02 (m, 3H), 6.56 (s, 2H), 5.77 (s, 2H), 5.22 - 5.00 (m, 1H), 3.55 (s, 2H), 3.50 - 3.47 (m, 2H), 3.12 - 3.08 (m, 1H), 3.06 - 3.01 (m, 1H), 2.76 (s, 3H). | 408.2 |
| 196 | 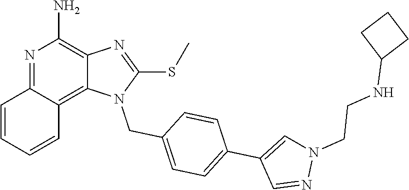 [SMILES: CSc1nc2c(N)nc3ccccc3c2n1Cc1ccc(-c2cnn(CCNC3CCC3)c2)cc1] | ¹H NMR (400 MHz, MeOD) δ 8.08-8.02 (m, 2H), 7.90 (s, 1H), 7.78-7.74 (m, 1H), 7.65 (t, J = 7.8 Hz, 1H), 7.59-7.55 (m, 2H), 7.41 (t, J = 7.8 Hz, 1H), 7.19-7.16 (m, 2H), 5.87 (s, 2H), 4.51-4.46 (m, 2H), 3.82-3.75 (m, 1H), 3.42 (t, J = 5.7 Hz, 2H), 2.89 (s, 3H), 2.34-2.17 (m, 4H), 1.96-1.86 (m, 2H). | 484.1 |
| 197 | 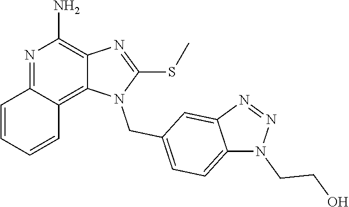 [SMILES: CSc1nc2c(N)nc3ccccc3c2n1Cc1ccc2c(c1)nnn2CCO] | ¹H NMR (400 MHz, DMSO-d₆) δ 7.87 (t, J = 7.6 Hz, 2H), 7.58 (dd, J = 13.1, 5.7 Hz, 2H), 7.34 (td, J = 8.5, 1.3 Hz, 2H), 7.07-7.00 (m, 1H), 6.61 (s, 2H), 5.97 (s, 2H), 4.97 (t, J = 5.5 Hz, 1H), 4.70 (t, J = 5.3 Hz, 2H), 3.84 (q, J = 5.3 Hz, 2H), 2.79 (s, 3H). | 406.2 |
| 198 | 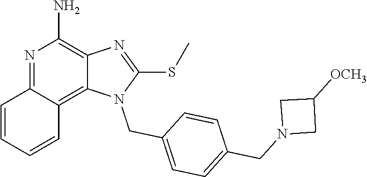 [SMILES: COC1CN(Cc2ccc(Cn3c(SC)nc4c(N)nc5ccccc5c43)cc2)C1] | ¹H NMR (400 MHz, DMSO-d₆) δ 7.81 (d, J = 8.1 Hz, 1H), 7.57 (d, J = 8.2 Hz, 1H), 7.35 (t, J = 7.5 Hz, 1H), 7.21 (d, J = 8.0 Hz, 2H), 7.05 (t, J = 9.1 Hz, 3H), 6.57 (s, 2H), 5.77 (s, 2H), 3.94 - 3.88 (m, 1H), 3.50 (s, 2H), 3.42 (d, J = 7.5 Hz, 2H), 3.11 (s, 3H), 2.80 (d, J = 6.0 Hz, 2H), 2.76 (s, 3H). | 420.1 |
| 199 | 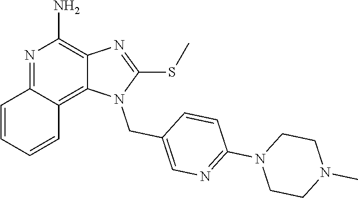 [SMILES: CSc1nc2c(N)nc3ccccc3c2n1Cc1ccc(N2CCN(C)CC2)nc1] | ¹H NMR (400 MHz, DMSO-d₆) δ 7.99-7.93 (m, 2H), 7.58 (d, J = 8.2 Hz, 1H), 7.38 (t, J = 7.6 Hz, 1H), 7.24 (d, J = 8.8 Hz, 1H), 7.14 (t, J = 7.4 Hz, 1H), 6.76 (d, J = 8.9 Hz, 1H), 6.57 (s, 2H), 5.67 (s, 2H), 3.70 (d, J = 31.7 Hz, 4H), 2.78 (s, 3H), 2.36 (s, 4H), 2.19 (s, 3H). | 420.2 |
| 200 | 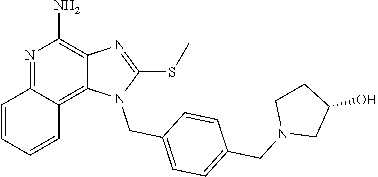 [SMILES: CSc1nc2c(N)nc3ccccc3c2n1Cc1ccc(CN2CC[C@H](O)C2)cc1] | ¹H NMR (400 MHz, MeOD) δ 7.97 (d, J = 8.1 Hz, 1H), 7.76 (d, J = 8.4 Hz, 1H), 7.65 (t, J = 7.8 Hz, 1H), 7.56 (dd, J = 8.0, 6.9 Hz, 2H), 7.42 - 7.36 (m, 1H), 7.30 (d, J = 8.1 Hz, 2H), 5.93 (s, 2H), 4.56 - 4.49 (m, 1H), 4.47 - 4.28 (m, 2H), 3.68 - 3.38 (m, 2H), 3.27 - 2.99 (m, 2H), 2.87 (s, 3H), 2.44 - 1.93 (m, 2H). | 420.2 |
| 201 | 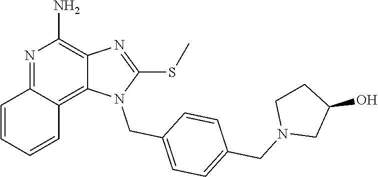 [SMILES: CSc1nc2c(N)nc3ccccc3c2n1Cc1ccc(CN2CC[C@@H](O)C2)cc1] | ¹H NMR (400 MHz, MeOD) δ 7.97 (d, J = 8.3 Hz, 1H), 7.76 (d, J = 8.4 Hz, 1H), 7.65 (t, J = 7.7 Hz, 1H), 7.56 (t, J = 7.2 Hz, 2H), 7.39 (t, J = 7.8 Hz, 1H), 7.30 (d, J = 8.0 Hz, 2H), 5.93 (s, 2H), 4.54 (s, 1H), 4.50-4.26 (m, 2H), 3.67-3.43 (m, 3H), 3.25-3.12 (m, 1H), 2.88 (s, 3H), 2.47-1.83 (m, 2H). | 420.2 |
| 202 | 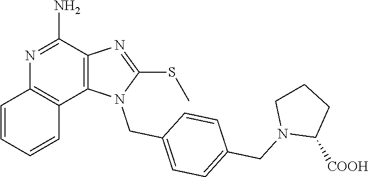 [SMILES: CSc1nc2c(N)nc3ccccc3c2n1Cc1ccc(CN2CCC[C@@H]2C(=O)O)cc1] | ¹H NMR (400 MHz, MeOD) δ 7.78 (d, J = 8.1 Hz, 1H), 7.63 (d, J = 8.2 Hz, 1H), 7.49 (d, J = 8.1 Hz, 2H), 7.39 (t, J = 7.3 Hz, 1H), 7.19 (d, J = 8.0 Hz, 2H), 7.09 (t, J = 7.4 Hz, 1H), 5.82 (s, 2H), 4.39 (d, J = 12.9 Hz, 1H), 4.12 (d, J = 12.9 Hz, 1H), 3.81 - 3.72 (m, 1H), 3.45 - 3.36 (m, 1H), 3.11 - 3.03 (m, 1H), 2.79 (s, 3H), 2.43 - 2.32 (m, 1H), 2.10 - 1.97 (m, 2H), 1.93 - 1.83 (m, 1H). | 448.2 |
| 203 | 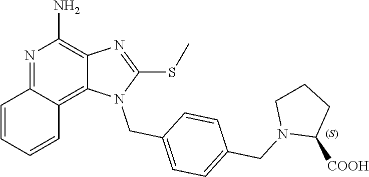 | ¹H NMR (400 MHz, DMSO-d₆) δ 7.81 (d, J = 7.6 Hz, 1H), 7.57 (d, J = 8.3 Hz, 1H), 7.34 (t, J = 9.1 Hz, 3H), 7.07 (dd, J = 15.2, 7.6 Hz, 3H), 6.58 (s, 2H), 5.79 (s, 2H), 3.99 (d, J = 13.2 Hz, 1H), 3.63 (d, J = 13.2 Hz, 1H), 3.26 - 3.25 (m, 3H), 2.99 - 2.92 (m, 1H), 2.77 (s, 3H), 2.11 - 1.99 (m, 1H), 1.84 - 1.83 (m, 1H), 1.77 - 1.61 (m, 2H). | 448.0 |
| 204 | 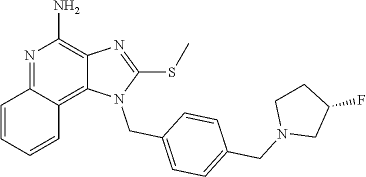 [SMILES: CSc1nc2c(N)nc3ccccc3c2n1Cc1ccc(CN2CC[C@H](F)C2)cc1] | ¹H NMR (400 MHz, MeOD) δ 7.98 (d, J = 8.2 Hz, 1H), 7.75 (d, J = 8.4 Hz, 1H), 7.65 (t, J = 7.8 Hz, 1H), 7.57 (t, J = 8.1 Hz, 2H), 7.39 (dd, J = 11.4, 4.2 Hz, 1H), 7.31 (d, J = 8.2 Hz, 2H), 5.94 (s, 2H), 5.43 - 5.41 (m, 1H), 4.51 - 4.39 (m, 2H), 3.75 - 3.57 (m, 2H), 3.55 - 3.35 (m, 2H), 2.88 (s, 3H), 2.69 - 2.16 (m, 2H). | 422.2 |
| 205 | | ¹H NMR (400 MHz, MeOD) δ 7.82 | 422.2 |
| | | (d, J = 8.2 Hz, 1H), 7.64 (d, J = 8.3 | |
| | | Hz, 1H), 7.42 - 7.36 (m, 1H), 7.31 | |
| | | (d, J = 8.1 Hz, 2H), 7.09 (t, J = 6.8 | |
| | | Hz, 3H), 5.80 (s, 2H), 5.22 - 5.00 | |
| | | (m, 1H), 3.60 (q, J = 12.8 Hz, 2H), | |
| | | 2.85 - 2.75 (m, 5H), 2.68 - 2.54 | |
| | | (m, 1H), 2.42 - 2.34 (m, 1H), 2.22 - | |
| | | 2.05 (m, 1H), 2.02 - 1.86 (m, | |
| | | 1H). | |
| 206 | | ¹H NMR (400 MHz, DMSO-d₆) δ | 406.2 |
| | | 13.97 (s, 1H), 10.88 (s, 1H), 8.81 | |
| | | (s, 1H), 7.96 (d, J = 8.2 Hz, 1H), | |
| | | 7.81 (d, J = 8.3 Hz, 1H), 7.64 (t, J = | |
| | | 7.8 Hz, 1H), 7.51 (d, J = 8.1 Hz, | |
| | | 2H), 7.38 (t, J = 7.8 Hz, 1H), 7.22 | |
| | | (d, J = 8.0 Hz, 2H), 6.26 (s, 1H), | |
| | | 5.90 (s, 2H), 4.45 (dd, J = 49.9, | |
| | | 43.0 Hz, 1H), 4.32 (d, J = 5.4 Hz, | |
| | | 2H), 4.10 (s, 2H), 3.81 (d, J = 9.6 | |
| | | Hz, 2H), 2.83 (s, 3H). | |
| 207 | 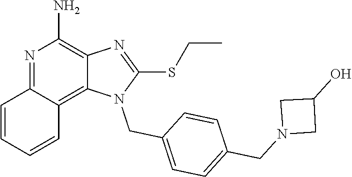 [SMILES: CCSc1nc2c(N)nc3ccccc3c2n1Cc1ccc(CN2CC(O)C2)cc1] | ¹H NMR (400 MHz, MeOD) δ 7.81 (d, J = 7.8 Hz, 1H), 7.64 (d, J = 8.2 Hz, 1H), 7.39 (t, J = 7.2 Hz, 1H), 7.25 (d, J = 8.1 Hz, 2H), 7.08 (dd, J = 11.0, 7.7 Hz, 3H), 5.84 (s, 2H), 4.30 (t, J = 6.3 Hz, 1H), 3.59 (s, 2H), 3.54 - 3.53 (m, 2H), 3.37 - 3.32 (m, 2H), 2.93 - 2.91 (m, 2H), 1.42 (t, J = 7.3 Hz, 3H). | 420.2 |
| 208 | 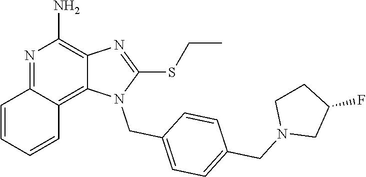 [SMILES: CCSc1nc2c(N)nc3ccccc3c2n1Cc1ccc(CN2CC[C@H](F)C2)cc1] | ¹H NMR (400 MHz, MeOD) δ 7.83 (d, J = 8.3 Hz, 1H), 7.64 (d, J = 8.4 Hz, 1H), 7.41 (t, J = 7.7 Hz, 1H), 7.32 (d, J = 8.0 Hz, 2H), 7.10 (dd, J = 14.1, 7.5 Hz, 3H), 5.86 (s, 2H), 5.12 (d, J = 55.4 Hz, 1H), 3.61 (q, J = 12.7 Hz, 2H), 3.37 - 3.34 (m, 2H), 2.87 - 2.75 (m, 2H), 2.63 - 2.61(m, 1H), 2.39 - 2.38 (m, 1H), 2.22 - 1.89 (m, 2H), 1.43 (t, J = 7.3 Hz, 3H). | 436.1 |
| 209 | 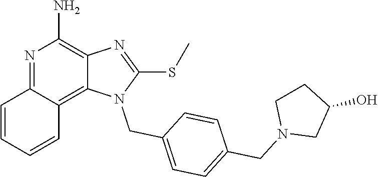 [SMILES: CSc1nc2c(N)nc3ccccc3c2n1Cc1ccc(CN2CC[C@H](O)C2)cc1] | ¹H NMR (400 MHz, MeOD) δ 7.84 (d, J = 8.2 Hz, 1H), 7.64 (d, J = 8.4 Hz, 1H), 7.43 - 7.37 (m, 1H), 7.32 (d, J = 8.1 Hz, 2H), 7.12 - 7.05 (m, 3H), 5.86 (s, 2H), 4.33 - 4.26 (m, 1H), 3.64 - 3.54 (m, 2H), 3.36 (d, J = 7.3 Hz, 2H), 2.77 - 2.64 (m, 2H), 2.53 - 2.39 (m, 2H), 2.14 - 2.04 (m, 1H), 1.72 - 1.61 (m, 1H), 1.43 (t, J = 7.3 Hz, 3H | 434.2 |
| 210 | 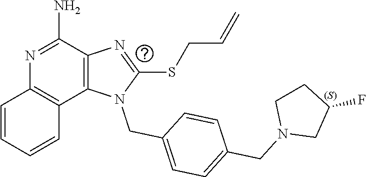 | ¹H NMR (400 MHz, MeOD) δ 7.97 (d, J = 7.9 Hz, 1H), 7.75 (d, J = 8.0 Hz, 1H), 7.65 (t, J = 7.4 Hz, 1H), 7.55 (d, J = 7.7 Hz, 2H), 7.39 (t, J = 7.8 Hz, 1H), 7.29 (d, J = 8.2 Hz, 2H), 6.06 - 6.05 (m, 1H), 5.96 (s, 2H), 5.52 - 5.31 (m, 2H), 5.17 (d, J = 10.0 Hz, 1H), 4.51 - 4.37 (m, 2H), 4.12 (d, J = 7.0 Hz, 2H), 3.83 - 3.59 (m, 2H), 3.50 - 3.35 (m, 2H), 2.70 - 2.15 (m, 2H). | 448.2 |
| 211 | 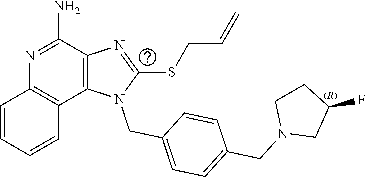 | ¹H NMR (400 MHz, MeOD) δ 7.83 (d, J = 7.9 Hz, 1H), 7.64 (d, J = 8.4 Hz, 1H), 7.40 (t, J = 7.8 Hz, 1H), 7.31 (d, J = 8.0 Hz, 2H), 7.12 - 7.05 (m, 3H), 5.99 - 5.98 (m, 1H), 5.88 (s, 2H), 5.25 (d, J = 16.1 Hz, 1H), 5.18 (s, 0.5H), 5.09 (d, J = 10.6 Hz, 1H), 5.04 (s, 0.5H), 3.95 (d, J = 7.1 Hz, 2H), 3.60 (q, J = 12.8 Hz, 2H), 2.86 - 2.74 (m, 2H), 2.69 - 2.55 (m, 1H), 2.42 - 2.34 (m, 1H), 2.22 - 2.06 (m, 1H), 2.03 - | 448.2 |
| | | 1.86 (m, 1H). | |
| 212 | | ¹H NMR (400 MHz, MeOD) δ 7.89 | 419.1 |
| | | (d, J = 7.6 Hz, 1H), 7.64 (d, J = 8.3 | |
| | | Hz, 1H), 7.41 (t, J = 7.7 Hz, 1H), | |
| | | 7.14 (t, J = 7.6 Hz, 1H), 7.02 (d, J = | |
| | | 8.7 Hz, 2H), 6.92 (d, J = 8.7 Hz, | |
| | | 2H), 5.73 (s, 2H), 3.21 - 3.08 (m, | |
| | | 4H), 2.79 (s, 3H), 2.62 - 2.53 (m, | |
| | | 4H), 2.32 (s, 3H). | |
| 213 | 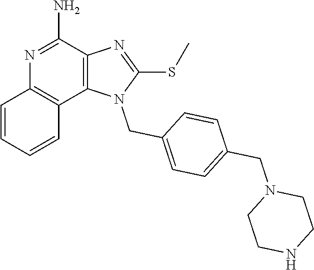 [SMILES: CSc1nc2c(N)nc3ccccc3c2n1Cc1ccc(CN2CCNCC2)cc1] | ¹H NMR (400 MHz, MeOD) δ 7.82 (d, J = 8.2 Hz, 1H), 7.64 (d, J = 8.4 Hz, 1H), 7.39 (t, J = 7.7 Hz, 1H), 7.30 (d, J = 8.1 Hz, 2H), 7.09 (d, J = 7.9 Hz, 3H), 5.81 (s, 2H), 3.46 (s, 2H), 2.87 - 2.72 (m, 7H), 2.40 (s, 4H). | 419.5 |
| 214 | 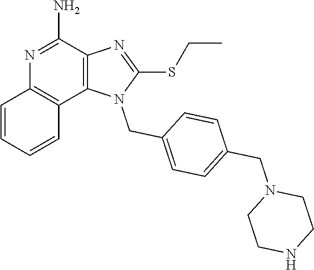 [SMILES: CCSc1nc2c(N)nc3ccccc3c2n1Cc1ccc(CN2CCNCC2)cc1] | ¹H NMR (400 MHz, MeOD) δ 7.86 - 7.79 (m, 1H), 7.64 (d, J = 8.4 Hz, 1H), 7.40 (t, J = 7.7 Hz, 1H), 7.30 (d, J = 6.0 Hz, 2H), 7.09 (t, J = 9.9 Hz, 3H), 5.85 (d, J = 5.7 Hz, 2H), 3.47 (d, J = 2.2 Hz, 2H), 3.38 - 3.33 (m, 2H), 2.82 (s, 4H), 2.41 (s, 4H), 1.43 (dd, J = 7.8, 6.8 Hz, 3H). | 433.2 |
| 215 | 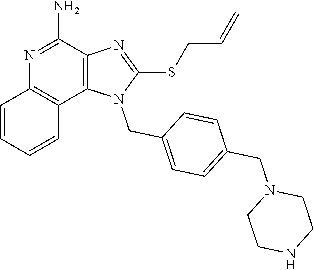 [SMILES: C=CCSc1nc2c(N)nc3ccccc3c2n1Cc1ccc(CN2CCNCC2)cc1] | ¹H NMR (400 MHz, MeOD) δ 7.98 (d, J = 8.4 Hz, 1H), 7.75 (d, J = 8.3 Hz, 1H), 7.68 - 7.59 (m, 3H), 7.40 (t, J = 7.8 Hz, 1H), 7.29 (d, J = 7.9 Hz, 2H), 6.12 - 6.00 (m, 1H), 5.95 (s, 2H), 5.40 (d, J = 16.9 Hz, 1H), 5.18 (d, J = 9.7 Hz, 1H), 4.38 (s, 2H), 4.12 (d, J = 7.1 Hz, 2H), 3.52 - 3.50 (m, 8H). | 445.2 |

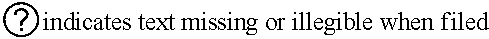

### Example 216: Preparation of 2-(methylthio)-1-({5-[(pyrrolidin-1-yl)methyl]thiophen-2-yl}methyl)-1H-imidazo[4,5-c]quinoline-4-amine

### Step 1: Preparation of 2-chloro-3-nitro-N-((5-(pyrrolidin-1-ylmethyl)thien-2-yl)methyl)quinoline-4-amine

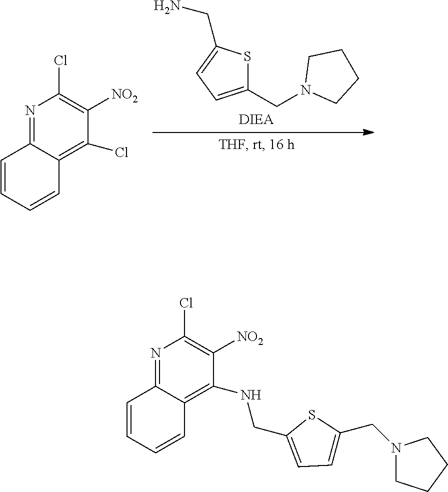 [SMILES: O=[N+]([O-])c1c(Cl)nc2ccccc2c1Cl ; NCc1ccc(CN2CCCC2)s1 ; O=[N+]([O-])c1c(Cl)nc2ccccc2c1NCc1ccc(CN2CCCC2)s1]

2,4-dichloro-3-nitroquinoline (400 mg, 1.65 mmol, 1.0 eq) was dissolved in tetrahydrofuran (5 mL). (5-(pyrrolidin-1-ylmethyl)thiophen-2-yl)methylamine (323.08 mg, 1.65 mmol, 1.0 eq) and N,N-diisopropylethylamine (640 mg, 4.959 mmol, 3.0 eq) were added. The mixture was stirred at 25° C. for 16 h. After the reaction was completed, as detected by LC-MS, the reaction mixture was concentrated to dryness and separated and purified by flash chromatography (0.1% aqueous formic acid solution:methanol=50%) to give the target compound (160 mg, yield: 24.3%). LC-MS (ESI) [M+H]⁺=403.1.

### Step 2: Preparation of N²,N²-bis(4-methoxybenzyl)-3-nitro N⁴-((5-(pyrrolidin-1-ylmethyl)thiophen-2 yl)methyl)quinoline-2,4-diamine

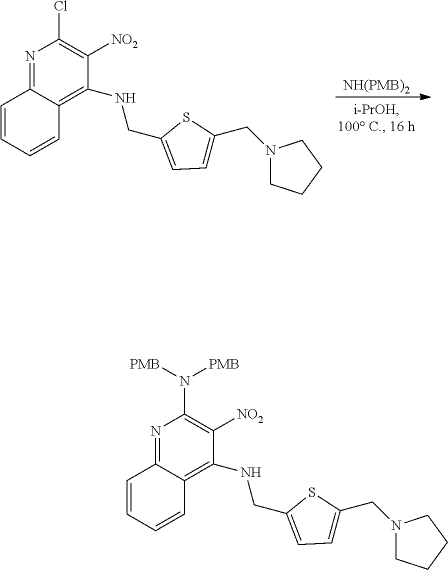

2-chloro-3-nitro-N-(5-(pyrrolidin-1-ylmethyl)thiophen-2-yl)methyl)quinoline-4-amine (400 mg, 0.99 mmol, 1.0 eq) was dissolved in isopropanol (3 mL), and bis(4-methoxybenzyl)amine (510.96 mg, 1.99 mmol, 2.0 eq) was added. The mixture was stirred at 100° C. for 16 h. After the reaction was completed, as detected by LC-MS, the reaction mixture was concentrated to dryness and separated and purified by flash chromatography (0.1% aqueous formic acid solution:methanol=80%) to give the target compound (300 mg, yield: 48.4%). LC-MS (ESI) [M+H]⁺=624.2.

### Step 3: Preparation of N²,N²-bis(4-methoxybenzyl) N⁴-((5-(pyrrolidin-1 ylmethyl)thiophen-2-yl)methyl)quinoline-2,3,4-triamine

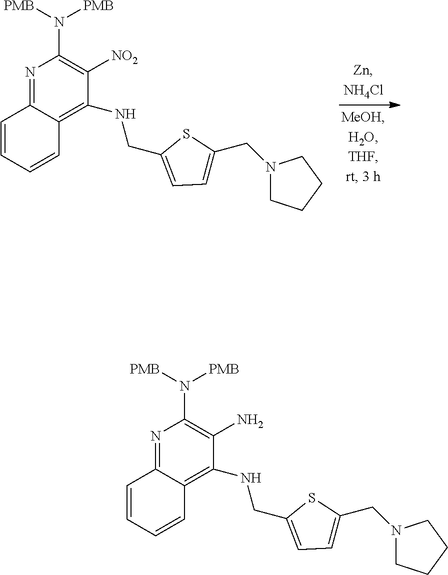

N²,N²-bis(4-methoxybenzyl)-3-nitro-N⁴-((5-(pyrrolidin-1 ylmethyl)thiophen-2-yl)methyl)quinoline-2,4-diamine (400 mg, 0.64 mmol, 1.0 eq) was dissolved in methanol (4 mL), water (2 mL), and tetrahydrofuran (4 mL). Ammonium chloride (171.17 mg, 3.20 mmol, 5 eq) and zinc powder (209.22 mg, 3.20 mmol, 5 eq) were added. The mixture was stirred at 25° C. for 3 h. After the reaction was completed, as detected by LC-MS, the reaction mixture was subjected to suction filtration and concentrated to dryness to give the target compound (260 mg, yield: 68.8%). LC-MS (ESI) [M+H]⁺=594.3.

### Step 4: Preparation of 4-{bis[(4-methoxyphenyl)methyl]amino}-1-({5-[(pyrrolidin-1-yl)methyl]thiophen-2-yl}methyl)-1,3-dihydro-2H-imidazo[4,5-c]quinoline-2-thione

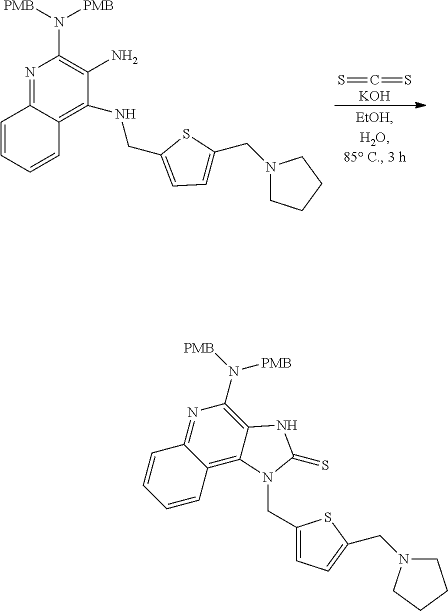

N²,N²-bis(4-methoxybenzyl)-N⁴-((5-(pyrrolidin-1 ylmethyl)thiophen-2 yl)methyl)quinoline-2,3,4-triamine (140 mg, 0.24 mmol, 1 eq) was dissolved in ethanol (5 mL) and water (0.5 mL). Potassium hydroxide (66.15 mg, 5 mmol, 5.0 eq) and carbon disulfide (0.09 mL, 1.18 mmol, 5 eq) were added. The mixture was stirred at 85° C. for 3 h. After the reaction was completed, as detected by LC-MS, the reaction mixture was concentrated by rotary evaporation, extracted with ethyl acetate (20 mL×2), and washed with saturated brine (30 mL×3). The organic phases were combined and concentrated by rotary evaporation to give the target product (120 mg, yield: 78.6%). LC-MS (ESI) [M+H]⁺=636.1.

### Step 5: N,N-bis[(4-methoxyphenyl)methyl]-2-(methylthio)-1-({5-[(pyrrolidin-1-yl)methyl]thiophen-2-yl}methyl)-1H-imidazo[4,5-c]quinoline-4-amine

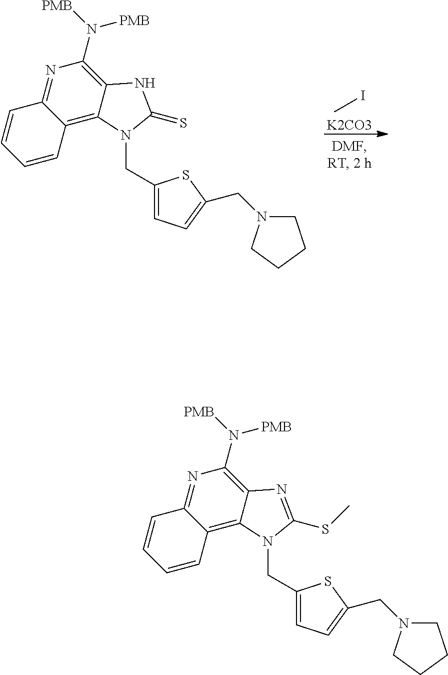

4-{bis[(4-methoxyphenyl)methyl]amino}-1-({5-[(pyrrolidin-1-yl)methyl]thiophen-2-yl}methyl)-1,3-dihydro-2H-imidazo[4,5-c]quinoline-2-thione (120.8 mg, 0.19 mmol, 1 eq) was dissolved in N,N-dimethylformamide (2 mL). Iodomethane (21.57 mg, 0.15 mmol, 0.8 eq) and potassium carbonate (39.39 mg, 0.28 mmol, 1.5 eq) were added sequentially. The mixture was stirred at 25° C. for 2 h. After the reaction was completed, as detected by LC-MS, the reaction mixture was extracted with ethyl acetate (2 mL×2), washed with saturated brine (2 mL×3), dried over anhydrous sodium sulfate, and concentrated by rotary evaporation to give the target product (100.0 mg, yield: 81%). LC-MS (ESI) [M+H]⁺=650.2.

### Step 6: Preparation of 2-(methylthio)-1-({5-[(pyrrolidin-1-yl)methyl]thiophen-2-yl}methyl)-1H-imidazo[4,5-c]quinoline-4-amine

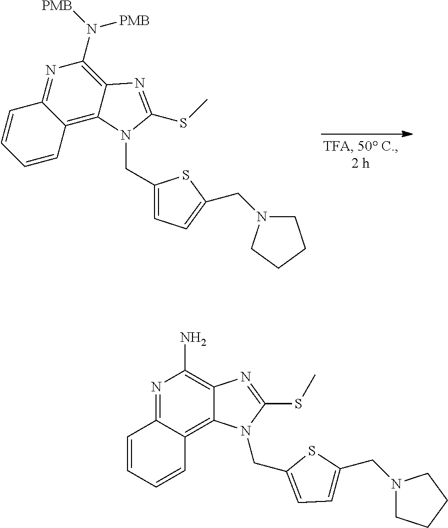

N,N-bis[(4-methoxyphenyl)methyl]-2-(methylthio)-1-({5-[(pyrrolidin-1-yl)methyl]thiophen-2-yl}methyl)-1H-imidazo[4,5-c]quinoline-4-amine (80 mg, 0.12 mmol, 1 eq) was dissolved in trifluoroacetic acid (5 mL). The reaction mixture was stirred at 50° C. for 2 h. After the reaction was completed, as detected by LC-MS, the reaction mixture was concentrated by rotary evaporation. The crude product was separated and purified by Prep-HPLC (C18, 0.01% aqueous ammonium bicarbonate monohydrate solution, MeCN) to give the target compound (15.34 mg, yield: 31.2%). LC-MS (ESI) [M+H]⁺=433.1; ¹H NMR (400 MHz, DMSO-d₆) δ 8.10 (d, J=8.0 Hz, 1H), 7.58 (d, J=8.2 Hz, 1H), 7.40 (t, J=7.4 Hz, 1H), 7.18 (t, J=7.3 Hz, 1H), 6.92 (d, J=3.3 Hz, 1H), 6.77 (d, J=3.3 Hz, 1H), 6.54 (s, 2H), 5.88 (s, 2H), 3.62 (s, 2H), 2.78 (s, 3H), 2.38 (s, 4H), 1.63 (s, 4H).

### Examples 217-251

The compounds of Examples 217/218/220 were prepared by referring to the preparation method of Example 216, and the compounds of the other examples were prepared by referring to the preparation method of Example 40, so that the compounds of Examples 217-251 were obtained.

| | | | LC-MS |
|---|---|---|---|
| | | | (ESI) |
| Example | Chemical structure | ¹H NMR | [M + H]⁺ |
| 217 | | ¹H NMR (400 MHz, MeOD) δ | 424.2 |
| | | 8.24 (d, J = 7.6 Hz, 1H), 7.77 | |
| | | (d, J = 7.5 Hz, 1H), 7.71 (t, J = | |
| | | 8.3 Hz, 1H), 7.52 (t, J = 7.7 | |
| | | Hz, 1H), 6.98 (s, 1H), 6.01 (s, | |
| | | 2H), 4.48 (s, 2H), 3.52-3.45 | |
| | | (m, 2H), 3.17-3.11 (m, 2H), | |
| | | 2.89 (s, 3H), 2.26 (s, 3H), 2.18- | |
| | | 2.08 (m, 2H), 2.01-1.90 (m, | |
| | | 2H). | |
| 218 | 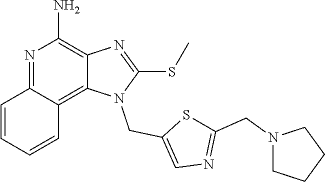 [SMILES: CSc1nc2c(N)nc3ccccc3c2n1Cc1cnc(CN2CCCC2)s1] | ¹H NMR (400 MHz, DMSO- d₆) δ 8.13 (d, J = 8.3 Hz, 1H), 7.72 (s, 1H), 7.59 (d, J = 7.8 Hz, 1H), 7.41 (t, J = 8.0 Hz, 1H), 7.21 (t, J = 7.7 Hz, 1H), 6.56 (s, 2H), 5.96 (s, 2H), 3.79 (s, 2H), 2.79 (s, 3H), 2.48 (s, 4H), 1.65 (s, 4H). | 411.2 |
| 219 | 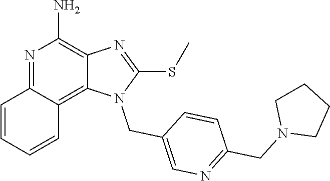 [SMILES: CSc1nc2c(N)nc3ccccc3c2n1Cc1ccc(CN2CCCC2)nc1] | ¹H NMR (400 MHz, MeOD) δ 8.60 (s, 1H), 8.04 (d, J = 8.1 Hz, 1H), 7.78 (d, J = 8.0 Hz, 1H), 7.73-7.62 (m, 2H), 7.52- 7.37 (m, 2H), 6.00 (s, 2H), 4.55 (s, 2H), 3.64 (s, 2H), 3.18 (s, 2H), 2.89 (s, 3H), 2.10 (d, J = 33.0 Hz, 4H). | 405.2 |
| 220 | 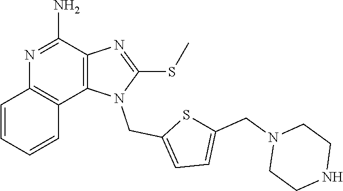 [SMILES: CSc1nc2c(N)nc3ccccc3c2n1Cc1ccc(CN2CCNCC2)s1] | ¹H NMR (400 MHz, MeOD) δ 8.07 (d, J = 8.3 Hz, 1H), 7.65 (d, J = 8.4 Hz, 1H), 7.47-7.42 (m, 1H), 7.24-7.19 (m, 1H), 6.89-6.79 (m, 2H), 5.90 (s, 2H), 3.60 (s, 2H), 2.81 (s, 3H), 2.80-2.75 (m, 4H), 2.39 (s, 4H). | 425.1 |
| 221 | 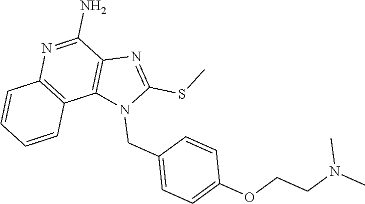 [SMILES: CSc1nc2c(N)nc3ccccc3c2n1Cc1ccc(OCCN(C)C)cc1] | ¹H NMR (400 MHz, DMSO- d₆) δ 7.85 (d, J = 7.7 Hz, 1H), 7.57 (d, J = 7.9 Hz, 1H), 7.36 (t, J = 7.3 Hz, 1H), 7.08 (t, J = 7.1 Hz, 1H), 7.03 (d, J = 8.7 Hz, 2H), 6.89 (d, J = 8.7 Hz, 2H), 6.55 (s, 2H), 5.72 (s, 2H), 3.96 (t, J = 5.8 Hz, 2H), 2.77 (s, 3H), 2.57 (t, J = 5.7 Hz, 2H), 2.17 (s, 6H). | 408.3 |
| 222 | 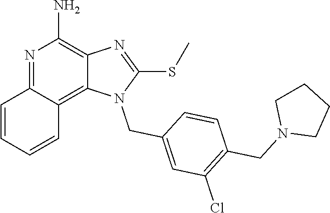 [SMILES: CSc1nc2c(N)nc3ccccc3c2n1Cc1ccc(CN2CCCC2)c(Cl)c1] | ¹H NMR (400 MHz, MeOD) δ 7.84 (d, J = 8.1 Hz, 1H), 7.68 (d, J = 8.4 Hz, 1H), 7.49-7.41 (m, 2H), 7.22 (s, 1H), 7.16 (t, J = 7.5 Hz, 1H), 7.04 (d, J = 8.0 Hz, 1H), 5.84 (s, 2H), 3.79 (s, 2H), 2.84 (s, 3H), 2.63 (s, 4H), 1.81 (s, 4H). | 438.1 |
| 223 | 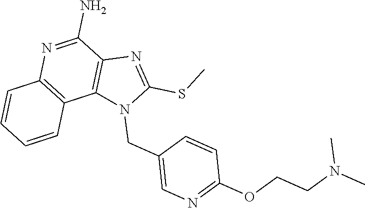 [SMILES: CSc1nc2c(N)nc3ccccc3c2n1Cc1ccc(OCCN(C)C)nc1] | ¹H NMR (400 MHz, MeOD) δ 7.98-7.90 (m, 2H), 7.66 (d, J = 8.4 Hz, 1H), 7.48-7.40 (m, 2H), 7.18 (t, J = 7.6 Hz, 1H), 6.76 (d, J = 8.6 Hz, 1H), 5.78 (s, 2H), 4.36 (t, J = 5.6 Hz, 2H), 2.81 (s, 3H), 2.71 (t, J = 5.5 Hz, 2H), 2.28 (s, 6H). | 409.2 |
| 224 | 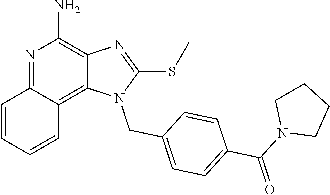 [SMILES: CSc1nc2c(N)nc3ccccc3c2n1Cc1ccc(C(=O)N2CCCC2)cc1] | ¹H NMR (400 MHz, DMSO- d₆) δ 14.30 (s, 1H), 8.91 (s, 2H), 7.99 (d, J = 8.3 Hz, 1H), 7.82 (d, J = 8.3 Hz, 1H), 7.65 (t, J = 7.7 Hz, 1H), 7.49 (d, J = 8.2 Hz, 2H), 7.40 (t, J = 7.7 Hz, 1H), 7.22 (d, J = 8.1 Hz, 2H), 5.92 (s, 2H), 3.42 (t, J = 6.7 Hz, 2H), 3.30 (t, J = 6.4 Hz, 2H), 2.84 (s, 3H), 1.87- 1.75 (m, 4H). | 418.2 |
| 225 | 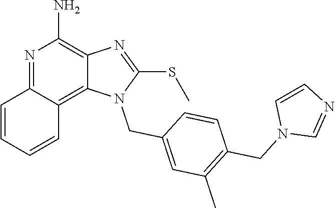 [SMILES: CSc1nc2c(N)nc3ccccc3c2n1Cc1ccc(Cn2ccnc2)c(C)c1] | ¹H NMR (400 MHz, MeOD) δ 8.94 (s, 1H), 7.98 (d, J = 7.9 Hz, 1H), 7.76 (d, J = 7.8 Hz, 1H), 7.68-7.62 (m, 1H), 7.59- 7.55 (m, 1H), 7.52 (d, J = 1.6 Hz, 1H), 7.43-7.36 (m, 1H), 7.22-7.13 (m, 2H), 7.03 (d, J = 7.8 Hz, 1H), 5.87 (s, 2H), 5.47 (s, 2H), 2.88 (s, 3H), 2.29 (s, 3H). | 415.9 |
| 226 | 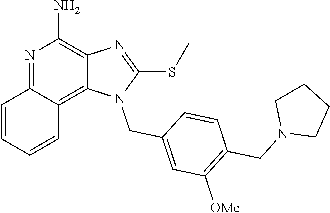 [SMILES: COc1cc(Cn2c(SC)nc3c(N)nc4ccccc4c32)ccc1CN1CCCC1] | ¹H NMR (400 MHz, MeOD) δ 7.88 (d, J = 8.3 Hz, 1H), 7.67 (d, J = 8.0 Hz, 1H), 7.43 (t, J = 7.7 Hz, 1H), 7.23 (d, J = 7.7 Hz, 1H), 7.13 (t, J = 7.7 Hz, 1H), 6.88 (s, 1H), 6.60 (d, J = 7.8 Hz, 1H), 5.83 (s, 2H), 3.75 (s, 3H), 3.69 (s, 2H), 2.83 (s, 3H), 2.69-2.53 (m, 4H), 1.87- 1.70 (m, 4H). | 434.2 |
| 227 | 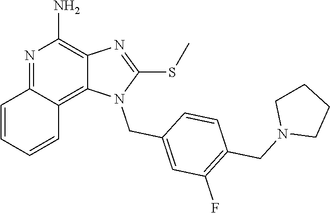 [SMILES: CSc1nc2c(N)nc3ccccc3c2n1Cc1ccc(CN2CCCC2)c(F)c1] | ¹H NMR (400 MHz, DMSO- d₆) δ 7.80 (d, J = 8.4 Hz, 1H), 7.57 (d, J = 8.0 Hz, 1H), 7.35 (dd, J = 13.1, 7.4 Hz, 2H), 7.08 (t, J = 7.5 Hz, 1H), 6.96 (d, J = 10.9 Hz, 1H), 6.83 (d, J = 8.5 Hz, 1H), 6.59 (s, 2H), 5.80 (s, 2H), 3.54 (s, 2H), 2.77 (s, 3H), 2.39 (s, 4H), 1.64 (s, 4H). | 422.1 |
| 228 | 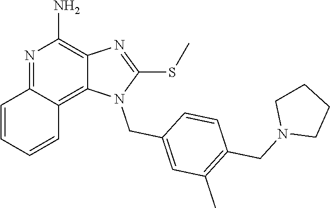 [SMILES: CSc1nc2c(N)nc3ccccc3c2n1Cc1ccc(CN2CCCC2)c(C)c1] | ¹H NMR (400 MHz, MeOD) δ 7.97 (d, J = 8.0 Hz, 1H), 7.75 (d, J = 7.9 Hz, 1H), 7.65 (t, J = 7.4 Hz, 1H), 7.48 (d, J = 7.9 Hz, 1H), 7.39 (t, J = 7.3 Hz, 1H), 7.18 (s, 1H), 7.07 (d, J = 7.4 Hz, 1H), 5.88 (s, 2H), 4.41 (s, 2H), 3.51 (s, 2H), 3.20 (d, J = 11.2 Hz, 2H), 2.88 (s, 3H), 2.42 (s, 3H), 2.18 (d, J = 7.0 Hz, 2H), 2.04-1.95 (m, 2H). | 418.3 |
| 229 | 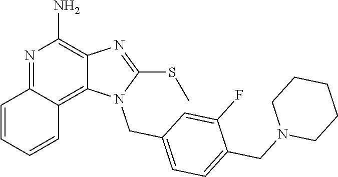 [SMILES: CSc1nc2c(N)nc3ccccc3c2n1Cc1ccc(CN2CCCCC2)c(F)c1] | ¹H NMR (400 MHz, DMSO- d₆) δ 7.80 (d, J = 8.1 Hz, 1H), 7.57 (d, J = 8.2 Hz, 1H), 7.34 (dt, J = 13.8, 7.5 Hz, 2H), 7.08 (t, J = 7.6 Hz, 1H), 6.96 (d, J = 10.9 Hz, 1H), 6.83 (d, J = 7.6 Hz, 1H), 6.59 (s, 2H), 5.80 (s, 2H), 3.39 (s, 2H), 2.77 (s, 3H), 2.27 (s, 4H), 1.47-1.39 (m, 4H), 1.33 (d, J = 4.7 Hz, 2H). | 436.2 |
| 230 | 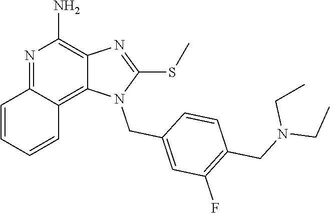 [SMILES: CCN(CC)Cc1ccc(Cn2c(SC)nc3c(N)nc4ccccc4c32)cc1F] | ¹H NMR (400 MHz, DMSO- d₆) δ 14.05 (s, 1H), 10.46 (s, 1H), 8.88 (s, 2H), 7.95 (d, J = 8.3 Hz, 1H), 7.83 (d, J = 8.4 Hz, 1H), 7.76 (t, J = 7.9 Hz, 1H), 7.66 (t, J = 7.7 Hz, 1H), 7.38 (dd, J = 18.8, 11.2 Hz, 1H), 7.26-7.21 (m, 1H), 7.09- 7.02 (m, 1H), 5.93 (s, 2H), 4.28 (d, J = 5.0 Hz, 2H), 3.04 (s, 4H), 2.84 (s, 3H), 1.23 (t, J = 7.2 Hz, 6H). | 424.2 |
| 231 | 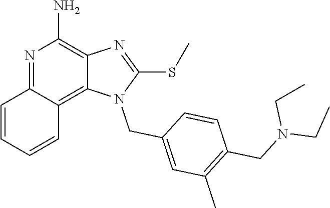 [SMILES: CCN(CC)Cc1ccc(Cn2c(SC)nc3c(N)nc4ccccc4c32)cc1C] | ¹H NMR (400 MHz, DMSO- d₆) δ 7.83 (s, 1H), 7.56 (s, 1H), 7.35 (s, 1H), 7.19 (s, 1H), 7.06 (s, 1H), 6.93 (s, 1H), 6.81 (s, 1H), 6.56 (s, 2H), 5.73 (s, 2H), 3.40 (s, 2H), 2.77 (s, 3H), 2.40 (s, 4H), 2.23 (s, 3H), 0.92 (s, 6H). | 420.0 |
| 232 | 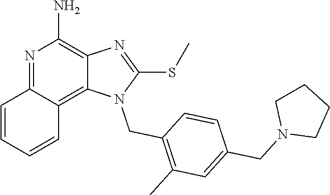 [SMILES: CSc1nc2c(N)nc3ccccc3c2n1Cc1ccc(CN2CCCC2)cc1C] | ¹H NMR (400 MHz, MeOD) δ 7.76 (d, J = 7.9 Hz, 1H), 7.67 (dd, J = 17.0, 8.2 Hz, 2H), 7.55 (s, 1H), 7.34 (t, J = 7.3 Hz, 1H), 7.20 (d, J = 7.1 Hz, 1H), 6.55 (d, J = 8.0 Hz, 1H), 5.85 (s, 2H), 4.31 (s, 2H), 3.50-3.40 (m, 2H), 3.14 (d, J = 8.8 Hz, 2H), 2.86 (s, 3H), 2.62 (s, 3H), 2.14 (s, 2H), 1.99 (s, 2H). | 418.2 |
| 233 | 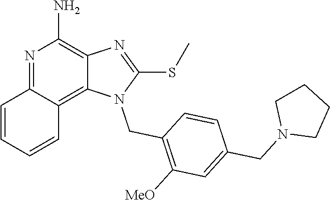 [SMILES: COc1cc(CN2CCCC2)ccc1Cn1c(SC)nc2c(N)nc3ccccc3c21] | ¹H NMR (400 MHz, MeOD) δ 7.65 (dd, J = 16.2, 7.5 Hz, 2H), 7.38 (dd, J = 11.9, 4.8 Hz, 1H), 7.16 (s, 1H), 7.06 (t, J = 7.0 Hz, 1H), 6.74 (d, J = 7.8 Hz, 1H), 6.42 (d, J = 7.8 Hz, 1H), 5.73 (s, 2H), 4.03 (s, 3H), 3.67 (s, 2H), 2.79 (s, 3H), 2.60 (s, 4H), 1.81 (s, 4H). | 434.3 |
| 234 | 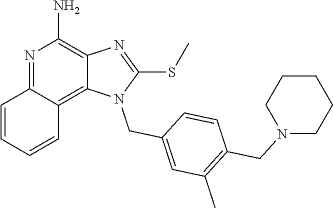 [SMILES: CSc1nc2c(N)nc3ccccc3c2n1Cc1ccc(CN2CCCCC2)c(C)c1] | ¹H NMR (400 MHz, MeOD) δ 7.96 (d, J = 8.1 Hz, 1H), 7.75 (d, J = 8.4 Hz, 1H), 7.64 (t, J = 7.8 Hz, 1H), 7.50 (d, J = 8.1 Hz, 1H), 7.38 (t, J = 7.8 Hz, 1H), 7.18 (s, 1H), 7.07 (d, J = 7.9 Hz, 1H), 5.88 (s, 2H), 4.30 (s, 2H), 3.43 (d, J = 11.6 Hz, 2H), 3.03 (t, J = 11.9 Hz, 2H), 2.87 (s, 3H), 2.41 (s, 3H), 1.81 (dq, J = 26.5, 12.6 Hz, 5H), 1.52 (t, J = 12.0 Hz, 1H). | 432.1 |
| 235 | 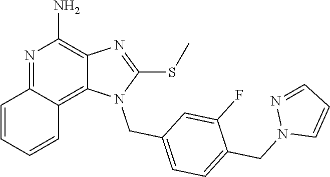 [SMILES: CSc1nc2c(N)nc3ccccc3c2n1Cc1ccc(Cn2cccn2)c(F)c1] | ¹H NMR (400 MHz, MeOD) δ 7.96 (d, J = 7.7 Hz, 1H), 7.74 (d, J = 7.6 Hz, 1H), 7.66 (m, 2H), 7.50 (d, J = 1.9 Hz, 1H), 7.44-7.38 (m, 1H), 7.09 (t, J = 7.8 Hz, 1H), 6.96 (m, 2H), 6.31 (t, J = 2.2 Hz, 1H), 5.87 (s, 2H), 5.38 (s, 2H), 2.87 (s, 3H). | 419.2 |
| 236 | 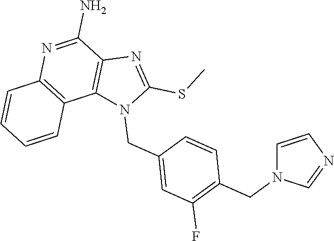 [SMILES: CSc1nc2c(N)nc3ccccc3c2n1Cc1ccc(Cn2ccnc2)c(F)c1] | ¹H NMR (400 MHz, DMSO- d₆) δ 14.03 (s, 1H), 9.24 (s, 1H), 7.96 (d, J = 8.2 Hz, 1H), 7.82 (d, J = 7.7 Hz, 1H), 7.68 (ddd, J = 18.9, 14.6, 4.5 Hz, 3H), 7.38 (dd, J = 13.9, 7.5 Hz, 2H), 7.21 (d, J = 11.1 Hz, 1H), 6.98 (d, J = 6.8 Hz, 1H), 5.90 (s, 2H), 5.48 (s, 2H), 2.82 (s, 3H). | 419.2 |
| 237 | 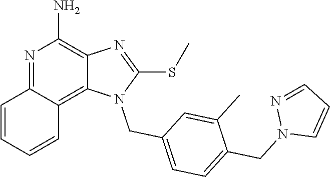 [SMILES: CSc1nc2c(N)nc3ccccc3c2n1Cc1ccc(Cn2cccn2)c(C)c1] | ¹H NMR (400 MHz, MeOD) δ 7.98 (d, J = 7.7 Hz, 1H), 7.73 (d, J = 7.6 Hz, 1H), 7.67-7.60 (m, 1H), 7.58 (d, J = 2.2 Hz, 1H), 7.52 (d, J = 1.6 Hz, 1H), 7.43-7.35 (m, 1H), 7.04 (s, 1H), 6.88 (dd, J = 25.7, 8.0 Hz, 2H), 6.32 (t, J = 2.2 Hz, 1H), 5.82 (s, 2H), 5.35 (s, 2H), 2.87 (s, 3H), 2.23 (s, 3H). | 415.2 |
| 238 | 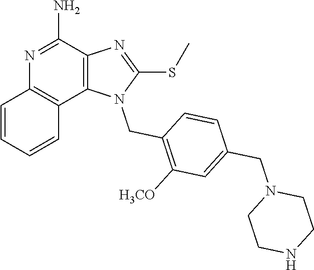 [SMILES: COc1cc(CN2CCNCC2)ccc1Cn1c(SC)nc2c(N)nc3ccccc3c21] | ¹H NMR (400 MHz, MeOD) δ 7.68-7.60 (m, 2H), 7.41- 7.35 (m, 1H), 7.14-7.03 (m, 2H), 6.71 (d, J = 7.8 Hz, 1H), 6.40 (d, J = 7.7 Hz, 1H), 5.70 (s, 2H), 4.02 (s, 3H), 3.47 (s, 2H), 2.82 (t, J = 4.9 Hz, 4H), 2.79 (s, 3H), 2.41 (s, 4H). | 449.1 |
| 239 | 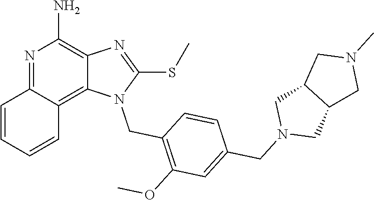 [SMILES: COc1cc(CN2C[C@H]3CN(C)C[C@H]3C2)ccc1Cn1c(SC)nc2c(N)nc3ccccc3c21] | ¹H NMR (400 MHz, CDCl₃) δ 7.97 (d, J = 8.3 Hz, 1H), 7.64- 7.51 (m, 2H), 7.30 (d, J = 8.2 Hz, 2H), 6.81 (d, J = 8.0 Hz, 1H), 6.55 (d, J = 7.7 Hz, 1H), 5.65 (s, 2H), 4.21 (s, 2H), 3.99 (s, 3H), 3.71-3.24 (m, 8H), 2.91 (s, 3H), 2.82 (s, 3H), 1.25- 1.24 (m, 2H). | 489.2 |
| 240 | 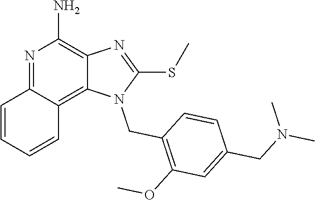 [SMILES: COc1cc(CN(C)C)ccc1Cn1c(SC)nc2c(N)nc3ccccc3c21] | ¹H NMR (400 MHz, MeOD) δ 7.63 (dd, J = 7.1, 6.0 Hz, 2H), 7.40-7.33 (m, 1H), 7.10 (d, J = 1.0 Hz, 1H), 7.08-7.00 (m, 1H), 6.68 (d, J = 7.7 Hz, 1H), 6.40 (d, J = 7.7 Hz, 1H), 5.69 (s, 2H), 4.02 (s, 3H), 3.41 (s, 2H), 2.78 (s, 3H), 2.20 (s, 6H). | 408.1 |
| 241 | 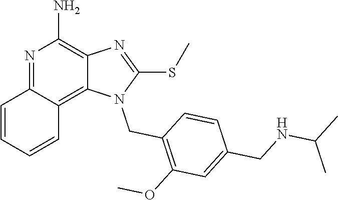 [SMILES: COc1cc(CNC(C)C)ccc1Cn1c(SC)nc2c(N)nc3ccccc3c21] | ¹H NMR (400 MHz, DMSO- d₆) δ 14.21 (s, 1H), 9.35 (s, 2H), 7.81 (t, J = 8.1 Hz, 2H), 7.68-7.62 (m, 2H), 7.41-7.37 (m, 1H), 7.02-6.96 (m, 1H), 6.61-6.56 (m, 1H), 5.73 (s, 2H), 4.10-4.04 (m, 2H), 3.99 (s, 3H), 3.27-3.19 (m, 1H), 2.81 (s, 3H), 1.29 (d, J = 6.5 Hz, 6H). | 422.1 |
| 242 | 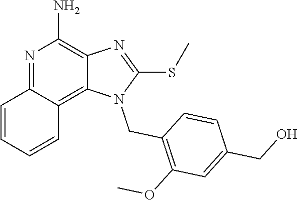 [SMILES: COc1cc(CO)ccc1Cn1c(SC)nc2c(N)nc3ccccc3c21] | ¹H NMR (400 MHz, MeOD) δ 7.67-7.60 (m, 2H), 7.41-7.34 (m, 1H), 7.13 (s, 1H), 7.08- 7.02 (m, 1H), 6.73 (d, J = 7.8 Hz, 1H), 6.40 (d, J = 7.8 Hz, 1H), 5.68 (s, 2H), 4.55 (s, 2H), 4.02 (s, 3H), 2.78 (s, 3H). | 318.4 |
| 243 | 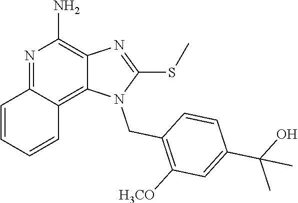 [SMILES: COc1cc(C(C)(C)O)ccc1Cn1c(SC)nc2c(N)nc3ccccc3c21] | ¹H NMR (400 MHz, MeOD) δ 7.69 (d, J = 8.0 Hz, 1H), 7.63 (d, J = 8.4 Hz, 1H), 7.39 (t, J = 7.6 Hz, 1H), 7.27 (s, 1H), 7.07 (t, J = 7.4 Hz, 1H), 6.83 (d, J = 7.9 Hz, 1H), 6.39 (d, J = 8.0 Hz, 1H), 5.70 (s, 2H), 4.03 (s, 3H), 2.79 (s, 3H), 1.48 (s, 6H). | 409.0 |
| 244 | 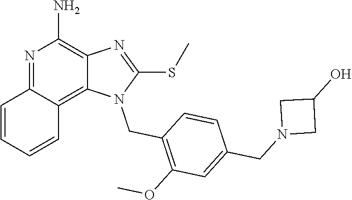 [SMILES: COc1cc(CN2CC(O)C2)ccc1Cn1c(SC)nc2c(N)nc3ccccc3c21] | ¹H NMR (400 MHz, MeOD) δ 7.83 (d, J = 8.3 Hz, 1H), 7.76 (d, J = 8.4 Hz, 1H), 7.64 (t, J = 7.8 Hz, 1H), 7.37 (t, J = 7.4 Hz, 1H), 7.33-7.28 (m, 1H), 6.95-6.89 (m, 1H), 6.68 (d, J = 7.7 Hz, 1H), 5.79 (s, 2H), 4.74-4.54 (m, 1H), 4.37 (d, J = 8.5 Hz, 2H), 4.34-4.25 (m, 2H), 4.05 (s, 3H), 3.98-3.88 (m, 2H), 2.86 (s, 3H). | 436.0 |
| 245 | 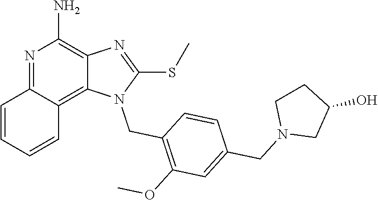 [SMILES: COc1cc(CN2CC[C@H](O)C2)ccc1Cn1c(SC)nc2c(N)nc3ccccc3c21] | ¹H NMR (400 MHz, MeOD) δ 7.69-7.59 (m, 2H), 7.41- 7.34 (m, 1H), 7.15 (s, 1H), 7.08-7.02 (m, 1H), 6.72 (d, J = 7.7 Hz, 1H), 6.39 (d, J = 7.8 Hz, 1H), 5.69 (s, 2H), 4.31 (tt, J = 6.6, 3.3 Hz, 1H), 4.02 (s, 3H), 3.58 (dd, J = 30.0, 12.8 Hz, 2H), 2.79 (s, 3H), 2.76- 2.65 (m, 2H), 2.53-2.41 (m, 2H), 2.15-2.04 (m, 1H), 1.69- 1.67 (m, 1H). | 450.5 |
| 246 | 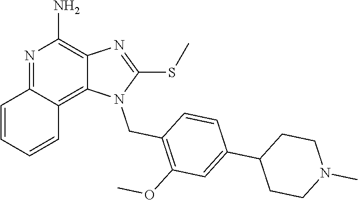 [SMILES: COc1cc(C2CCN(C)CC2)ccc1Cn1c(SC)nc2c(N)nc3ccccc3c21] | ¹H NMR (400 MHz, MeOD) δ 7.69 (d, J = 7.8 Hz, 1H), 7.64 (d, J = 8.3 Hz, 1H), 7.39 (t, J = 7.7 Hz, 1H), 7.08 (t, J = 7.6 Hz, 1H), 6.98 (s, 1H), 6.64 (d, J = 7.9 Hz, 1H), 6.38 (d, J = 7.9 Hz, 1H), 5.70 (s, 2H), 4.00 (s, 3H), 2.97 (d, J = 11.5 Hz, 2H), 2.78 (s, 3H), 2.52 (dd, J = 13.8, 9.9 Hz, 1H), 2.31 (s, 3H), 2.13 (dd, J = 11.8, 9.5 Hz, 2H), 1.86-1.69 (m, 4H). | 448.1 |
| 247 | 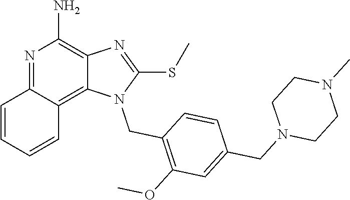 [SMILES: COc1cc(CN2CCN(C)CC2)ccc1Cn1c(SC)nc2c(N)nc3ccccc3c21] | ¹H NMR (400 MHz, MeOD) δ 7.69 (d, J = 7.5 Hz, 1H), 7.64 (d, J = 8.1 Hz, 1H), 7.39 (t, J = 7.6 Hz, 1H), 7.13 (s, 1H), 7.07 (t, J = 7.3 Hz, 1H), 6.72 (d, J = 7.6 Hz, 1H), 6.41 (d, J = 7.9 Hz, 1H), 5.73 (s, 2H), 4.02 (s, 3H), 3.49 (s, 2H), 2.79 (s, 3H), 2.47 (br s, 8H), 2.26 (s, 3H). | 463.2 |
| 248 | 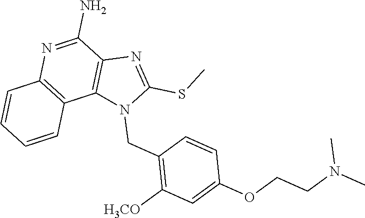 [SMILES: COc1cc(OCCN(C)C)ccc1Cn1c(SC)nc2c(N)nc3ccccc3c21] | ¹H NMR (400 MHz, MeOD) δ 7.74 (d, J = 8.4 Hz, 1H), 7.65 (d, J = 8.1 Hz, 1H), 7.43 (t, J = 7.4 Hz, 1H), 7.13 (t, J = 7.3 Hz, 1H), 6.72 (d, J = 2.1 Hz, 1H), 6.43-6.34 (m, 2H), 5.66 (s, 2H), 4.11 (t, J = 5.3 Hz, 2H), 3.97 (s, 3H), 2.96 (t, J = 5.3 Hz, 2H), 2.80 (s, 3H), 2.49 (s, 6H). | 438.2 |
| 249 | 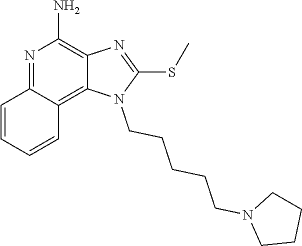 [SMILES: CSc1nc2c(N)nc3ccccc3c2n1CCCCCN1CCCC1] | ¹H NMR (400 MHz, DMSO- d₆) δ 8.01 (d, J = 8.0 Hz, 1H), 7.61 (d, J = 8.6 Hz, 1H), 7.43 (s, 1H), 7.25 (s, 1H), 6.48 (s, 2H), 4.47 (s, 2H), 2.77 (s, 3H), 2.34 (s, 6H), 1.84 (s, 2H), 1.63 (s, 4H), 1.45 (s, 4H). | 370.2 |
| 250 | 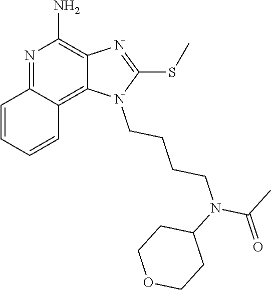 [SMILES: CSc1nc2c(N)nc3ccccc3c2n1CCCCN(C(C)=O)C1CCOCC1] | ¹H NMR (400 MHz, MeOD) δ 8.08 (t, J = 7.9 Hz, 1H), 7.69 (d, J = 8.5 Hz, 1H), 7.49 (t, J = 7.7 Hz, 1H), 7.34 (t, J = 7.2 Hz, 1H), 4.63-4.53 (m, 2H), 3.97-3.89 (m, 2H), 3.48- 3.34 (m, 3H), 3.28-3.23 (m, 2H), 2.86-2.78 (m, 3H), 2.07 (d, J = 23.3 Hz, 3H), 2.01- 1.92 (m, 2H), 1.82-1.65 (m, 4H), 1.60-1.44 (m, 2H). | 428.1 |
| 251 | 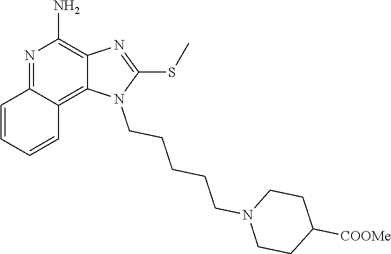 [SMILES: COC(=O)C1CCN(CCCCCn2c(SC)nc3c(N)nc4ccccc4c32)CC1] | ¹H NMR (400 MHz, MeOD) δ 8.06 (d, J = 8.1 Hz, 1H), 7.69 (d, J = 8.4 Hz, 1H), 7.49 (t, J = 7.3 Hz, 1H), 7.34 (t, J = 7.4 Hz, 1H), 4.55 (t, J = 7.4 Hz, 2H), 3.66 (s, 3H), 2.86 (d, J = 11.7 Hz, 2H), 2.81 (s, 3H), 2.34 (dd, J = 15.1, 7.6 Hz, 3H), 2.04 (dd, J = 20.2, 8.7 Hz, 2H), 1.96 (dd, J = 14.8, 7.3 Hz, 2H), 1.88 (d, J = 13.2 Hz, 2H), 1.76- 1.62 (m, 2H), 1.61-1.51 (m, 2H), 1.48 (dd, J = 14.5, 7.5 Hz, 2H). | 442.2 |

### Example 252: Preparation of 4-amino-2-butoxy-S-(4-(4-((methylamino)methyl)phenoxy)benzyl)-7,8-dihydropteridin-6(5H)-one

### Step 1: Preparation of tert-butyl (4-hydroxybenzyl)carbamate

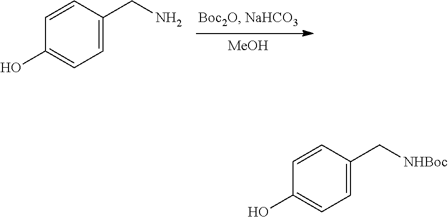 [SMILES: "MeOH" ; NCc1ccc(O)cc1 ; CC(C)(C)OC(=O)NCc1ccc(O)cc1]

4-(aminomethyl)phenol (25.0 g, 203 mmol, 1.0 eq) was dissolved in methanol (300 mL). Sodium bicarbonate (35.8 g, 426 mmol, 2.1 eq) and di-tert-butyl dicarbonate (48.7 g, 223 mmol, 1.1 eq) were added. The reaction mixture was stirred at 70° C. for 16 h. After the reaction was completed, as detected by TLC (PE:EA=3:1, 254 nm), the reaction mixture was filtered and concentrated to give a crude product, which was separated and purified by flash chromatography (silica gel, PE:EA=100:0 to 70:30) to give the target compound (33.0 g, yield: 72.8%). ¹H NMR (400 MHz, CDCl₃): δ 7.09-7.07 (m, 2H), 6.79-6.75 (m, 2H), 4.93 (brs, 1H), 4.20 (d, J=4.8 Hz, 2H), 1.46 (s, 9H).

### Step 2: Preparation of tert-butyl (4-(4-cyanophenoxy)benzyl)carbamate

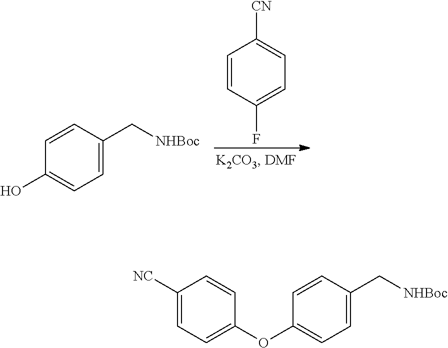 [SMILES: CC(C)(C)OC(=O)NCc1ccc(O)cc1 ; N#Cc1ccc(F)cc1 ; CC(C)(C)OC(=O)NCc1ccc(Oc2ccc(C#N)cc2)cc1]

Tert-butyl (4-hydroxybenzyl)carbamate (33.0 g, 147 mmol, 1.0 eq) was dissolved in N,N-dimethylformamide (500 mL). p-fluorobenzonitrile (17.9 g, 148 mmol, 1.0 eq) and potassium carbonate (24.5 g, 177 mmol, 1.2 eq) were added. The mixture was stirred at 120° C. for 16 h. After the reaction was completed, as detected by TLC (PE:EA=3:1, 254 nm), the reaction mixture was added with water (500 mL) for dilution and extracted with ethyl acetate (300 mL×3). The organic phases were combined, washed with saturated brine, dried over anhydrous sodium sulfate, filtered and concentrated to give a crude product, which was separated and purified by flash chromatography (silica gel, PE:EA=100:0 to 70:30) to give the target compound (29.0 g, yield: 60.5%). ¹H NMR (400 MHz, CDCl₃): δ 7.59 (d, J=11.6 Hz, 2H), 7.32 (d, J=8.4 Hz, 2H), 7.03-6.98 (m, 4H), 4.33-4.32 (m, 2H), 1.47 (s, 9H).

### Step 3: Preparation of tert-butyl (4-(4-cyanophenoxy)benzyl)(methyl)carbamate

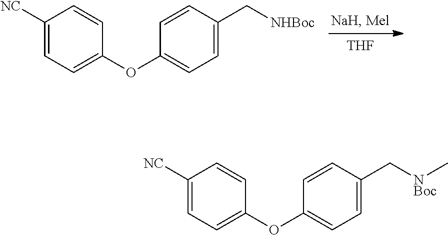 [SMILES: CC(C)(C)OC(=O)NCc1ccc(Oc2ccc(C#N)cc2)cc1 ; CN(Cc1ccc(Oc2ccc(C#N)cc2)cc1)C(=O)OC(C)(C)C ; "THF"]

Tert-butyl (4-(4-cyanophenoxy)benzyl)carbamate (28.0 g, 86.3 mmol, 1.0 eq) was dissolved in tetrahydrofuran (300 mL), and then sodium hydride (5.18 g, 129 mmol, purity: 60%, 1.5 eq) was added at 0° C. After the reaction mixture was stirred at 0° C. for 30 min under nitrogen atmosphere, iodomethane (18.4 g, 8.06 mL, 129 mmol, 1.5 eq) was added dropwise to the reaction mixture, which was stirred at 25° C. for 2 h. After the reaction was completed, as detected by TLC (PE:EA=3:1, 254 nm), the reaction system was added with water (500 mL) for dilution and extracted with ethyl acetate (200 mL×3). The organic phases were combined, washed with saturated brine, dried over anhydrous sodium sulfate, filtered and concentrated to give a crude product, which was separated and purified by flash chromatography (silica gel, PE:EA=100:0 to 66:34) to give the target compound (28.0 g, yield: 95.9%). ¹H NMR (400 MHz, CDCl₃): δ 7.51 (d, J=8.8 Hz, 2H), 7.19 (d, J=8.0 Hz, 2H), 6.96-6.91 (m, 4H), 4.35 (brs, 2H), 2.77 (s, 3H), 1.41 (s, 9H).

### Step 4: Preparation of tert-butyl (4-(4-(aminomethyl)phenoxy)benzyl)(methyl)carbamate

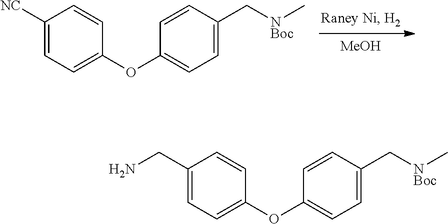 [SMILES: "MeOH" ; CN(Cc1ccc(Oc2ccc(C#N)cc2)cc1)C(=O)OC(C)(C)C ; CN(Cc1ccc(Oc2ccc(CN)cc2)cc1)C(=O)OC(C)(C)C]

Tert-butyl (4-(4-cyanophenoxy)benzyl)(methyl)carbamate (28.0 g, 82.7 mmol, 1.0 eq) was dissolved in methanol (300 mL). Raney nickel (3 g) was added under nitrogen atmosphere. The reaction mixture was purged with nitrogen 3 times and then purged with hydrogen 3 times. The reaction mixture was stirred at 25° C. for 16 h under hydrogen atmosphere (15 Psi). After the reaction was completed, as detected by TLC (DCM:MeOH=10:1, 254 nm), the reaction mixture was filtered through celite and concentrated to give a crude product, which was separated and purified by flash chromatography (silica gel, DCM:MeOH=100:0 to 94:6) to give the target compound (19.0 g, yield: 67.1%). ¹H NMR (400 MHz, CDCl₃): δ 7.20 (d, J=8.0 Hz, 2H), 7.10 (d, J=8.4 Hz, 2H), 6.91-6.86 (m, 4H), 4.31 (brs, 2H), 3.77 (brs, 2H), 2.74 (brs, 3H), 1.41 (s, 9H).

### Step 5: Preparation of ethyl (4-(4-(((tert-butoxycarbonyl)(methyl)amino)methyl)phenoxy)benzyl)glycinate

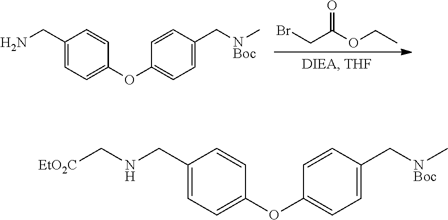

Tert-butyl (4-(4-(aminomethyl)phenoxy)benzyl)(methyl)carbamate (8.60 g, 55.3 mmol, 1.0 eq) was dissolved in tetrahydrofuran (100 mL), and then N,N-diisopropylethylamine (7.14 g, 1.41 mmol, 2.15 eq) was added. Ethyl bromoacetate (3.36 g, 20.1 mmol, 0.8 eq) was slowly added dropwise to the reaction mixture at 0° C. The reaction mixture was stirred at 25° C. for 16 h. After the starting material was consumed completely, as detected by TLC (DCM:MeOH=10:1, 254 nm) and LC-MS, the reaction mixture was poured into ice water (150 mL) and extracted with ethyl acetate (150 mL×2). The organic phases were combined, washed with saturated brine, dried over anhydrous sodium sulfate, filtered and concentrated to give a crude product, which was separated and purified by flash chromatography (silica gel, PE:EA=100:0 to 67:33) to give the target compound (8.60 g, yield: 71.9%). LC-MS (ESI) [M+H]⁺=429.17; ¹H NMR (400 MHz, CDCl₃): δ 7.30 (d, J=8.4 Hz, 2H), 7.17 (d, J=8.0 Hz, 2H), 6.97-6.94 (m, 4H), 4.39 (brs, 2H), 4.20 (q, J=8.0 Hz, 2H), 3.78 (s, 2H), 3.41 (s, 2H), 2.82 (brs, 3H), 1.48 (s, 9H), 1.28 (t, J=8.0 Hz, 3H).

### Step 6: Preparation of ethyl N-(6-amino-2-(methylthio)-5-nitropyrimidin-4-yl)-N-(4-(4-(((tert-butoxycarbonyl)(methyl)amino)methyl)phenoxy)benzyl)glycinate

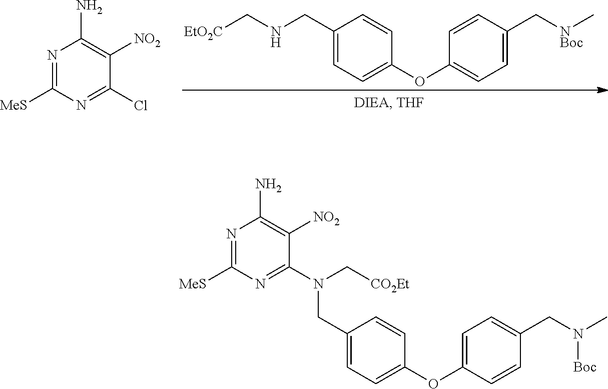 [SMILES: CSc1nc(N)c([N+](=O)[O-])c(Cl)n1 ; CCOC(=O)CNCc1ccc(Oc2ccc(CN(C)C(=O)OC(C)(C)C)cc2)cc1 ; CCOC(=O)CN(Cc1ccc(Oc2ccc(CN(C)C(=O)OC(C)(C)C)cc2)cc1)c1nc(SC)nc(N)c1[N+](=O)[O-]]

6-chloro-2-(methylthio)-5-nitropyrimidin-4-amine (2.90 g, 13.1 mmol, 1.0 eq) was dissolved in tetrahydrofuran (30 mL), and then ethyl (4-(4-(((tert-butoxycarbonyl)(methyl)amino)methyl)phenoxy)benzyl)glycinate (6.20 g, 14.5 mmol, 1.1 eq) and N,N-diisopropylethylamine (5.10 g, 39.4 mmol, 3.0 eq) were added. The reaction mixture was stirred at 25° C. for 3 h. After the starting material was consumed completely, as detected by TLC (PE:EA=3:1, 254 nm), the reaction mixture was filtered. The filtrate was washed with saturated brine, dried over anhydrous sodium sulfate, filtered and concentrated to give a crude product, which was separated and purified by flash chromatography (silica gel, PE:EA=100:0 to 67:33) to give the target compound (6.40 g, yield: 85.5%). ¹H NMR (400 MHz, CDCl₃): δ 7.27 (d, J=7.6 Hz, 2H), 7.19 (d, J=8.0 Hz, 2H), 6.96-6.94 (m, 4H), 4.69 (s, 2H), 4.39 (brs, 2H), 4.23-4.17 (q, J=5.2 Hz, 2H), 4.07 (s, 2H), 2.82 (brs, 3H), 2.41 (s, 3H), 1.48 (s, 9H), 1.27 (t, J=5.2 Hz, 3H).

### Step 7: Preparation of ethyl N-(6-amino-2-(methylsulfonyl)-5-nitropyrimidin-4-yl)-N-(4-(4-(((tert-butoxycarbonyl)(methyl)amino)methyl)phenoxy)benzyl)glycinate

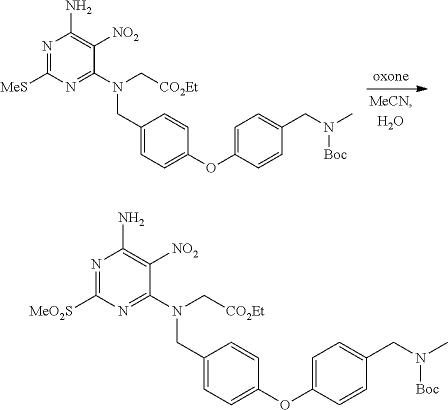 [SMILES: CCOC(=O)CN(Cc1ccc(Oc2ccc(CN(C)C(=O)OC(C)(C)C)cc2)cc1)c1nc(SC)nc(N)c1[N+](=O)[O-] ; CCOC(=O)CN(Cc1ccc(Oc2ccc(CN(C)C(=O)OC(C)(C)C)cc2)cc1)c1nc(S(C)(=O)=O)nc(N)c1[N+](=O)[O-]]

Ethyl N-(6-amino-2-(methylthio)-5-nitropyrimidin-4-yl)-N-(4-(4-(((tert-butoxycarbonyl)(methyl)amino)methyl)phenoxy)benzyl)glycinate (1.00 g, 1.95 mmol, 1.0 eq) was dissolved in acetonitrile (12 mL), and then an aqueous solution (12 mL) of potassium peroxymonosulfonate (3.00 g, 4.88 mmol, 2.5 eq) was added. The reaction mixture was stirred at 25° C. for 16 h. After the starting material was consumed completely, as detected by LC-MS, the reaction mixture was filtered. The filtrate was extracted with ethyl acetate (15 mL×2). The organic phases were combined, washed with saturated brine, dried over anhydrous sodium sulfate, and filtered. n-butanol (8 mL) was added to the filtrate, which was then concentrated to give a crude product of the target compound (800 mg, 63.6%). The crude product was used directly in the next step without purification. LC-MS (ESI) [M−56+H]⁺=588.8; ¹H NMR (400 MHz, CDCl₃): δ 7.24 (d, J=8.0 Hz, 2H), 7.20 (d, J=8.0 Hz, 2H), 6.95 (d, J=8.4 Hz, 4H), 4.68 (brs, 2H), 4.40 (brs, 2H), 4.15-4.09 (m, 2H), 2.93 (s, 3H), 2.04 (s, 2H), 1.78 (brs, 3H), 1.48 (s, 9H), 1.27 (t, J=5.2 Hz, 3H).

### Step 8: Preparation of ethyl N-(6-amino-2-butoxy-5-nitropyrimidin-4-yl)-N-(4-(4-(((tert-butoxycarbonyl)(methyl)amino)methyl)phenoxy)benzyl)glycine

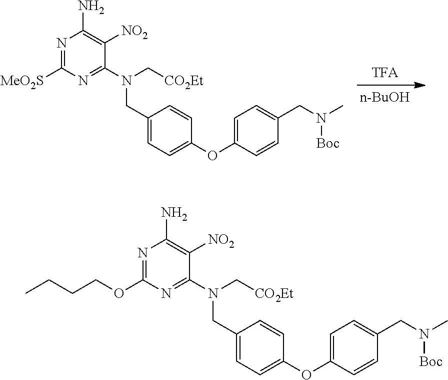

Ethyl N-(6-amino-2-(methyl sulfonyl)-5-nitropyrimidin-4-yl)-N-(4-(4-(((tert-butoxycarbonyl)(methyl)amino)methyl)phenoxy)benzyl)glycinate (800 mg, 1.24 mmol, 1.0 eq) was dissolved in n-butanol (8 mL), and then trifluoroacetic acid (425 mg, 3.72 mmol, 3 eq) was added. The reaction mixture was stirred at 100° C. for 4 h. After the starting material was consumed completely, as detected by LC-MS, the reaction mixture was adjusted to pH=8 with a saturated sodium bicarbonate solution, extracted with ethyl acetate (10 mL×2), washed with saturated brine, dried over anhydrous sodium sulfate, filtered and concentrated to give a crude product, which was separated and purified by flash chromatography (silica gel, PE:EA=100:0 to 67:33) to give the target compound (260 mg, yield: 32.8%). LC-MS (ESI) [M+H]⁺=640.3; ¹H NMR (400 MHz, CDCl₃): δ 7.27 (d, J=9.2 Hz, 2H), 7.19 (d, J=8.0 Hz, 2H), 6.96-6.94 (m, 4H), 4.69 (s, 2H), 4.39 (brs, 2H), 4.21-4.20 m, 2H), 4.15-4.09 (m, 2H), 4.05 (s, 2H), 2.82 (brs, 3H), 1.71-1.59 (m, 2H), 1.48 (s, 9H), 1.44-1.40 (m, 2H), 1.27 (t, J=3.6 Hz, 3H), 0.93 (t, J=7.6 Hz, 3H).

### Step 9: Preparation of tert-butyl (4-(4-((4-amino-2-butoxy-6-oxo-6,7-dihydropteridin-8(5H)-yl)methyl)phenoxy)benzyl)(methyl)carbamate

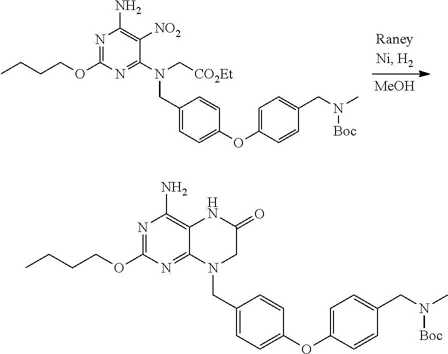

Ethyl N-(6-amino-2-butoxy-5-nitropyrimidin-4-yl)-N-(4-(4-(((tert-butoxycarbonyl)(methyl)amino)methyl)phenoxy)benzyl)glycinate (260 mg, 407 μcool, 1.0 eq) was dissolved in methanol (5 mL), and then Raney nickel (50.0 mg) was added under nitrogen atmosphere. The reaction mixture was purged with nitrogen 3 times and purged with hydrogen 3 times. The reaction mixture was stirred at 25° C. for 2 h under hydrogen atmosphere (15 Psi). After the starting material was consumed completely, as detected by LC-MS, the reaction mixture was filtered through celite and concentrated to give a crude product of the target compound (170 mg, yield: 74.2%), which was used directly in the next step without purification. LC-MS (ESI) [M+H]+=564.3.

### Step 10: Preparation of 4-amino-2-butoxy-8-(4-(4-((methylamino)methyl)phenoxy)benzyl)-7,8-dihydropteridin-6(5H)-one

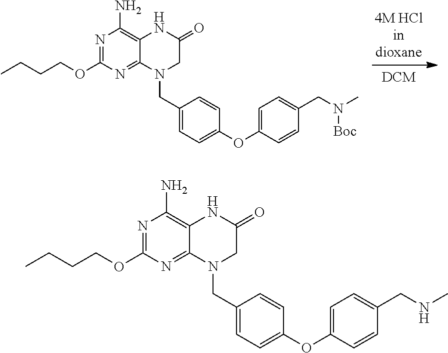

Tert-butyl (4-(4-((4-amino-2-butoxy-6-oxo-6,7-dihydropteridin-8(511)-yl)methyl)phenoxy)benzyl)(methyl)carbamate (170 mg, 266 μcool, 1.0 eq) was dissolved in dichloromethane (3 mL) and a solution of hydrochloric acid in dioxane (1 mL, 4M). The mixture was stirred at 25° C. for 3 h. After the starting material was consumed completely, as detected by LC-MS, the reaction mixture was concentrated to give a crude product, which was separated and purified by Prep-HPLC (C18, 0.01% aqueous formic acid solution, MeCN) to give the target compound (11.3 mg, yield: 9.18%). LC-MS (ESI) [M+H]⁺=463.2; ¹H NMR (400 MHz, DMSO-d₆): δ 9.81 (s, 1H), 8.78 (s, 1H), 7.47 (d, J=7.6 Hz, 2H), 7.37 (d, J=8.0 Hz, 2H), 7.06-7.00 (m, 4H), 4.69 (s, 2H), 4.17-4.09 (m, 4H), 3.90 (s, 2H), 2.55 (s, 3H), 1.62-1.59 (m, 2H), 1.37-1.32 (m, 2H), 0.90-0.86 (t, J=6.8 Hz, 3H).

### Comparative Example 1: Preparation of 2-methoxy-1-((4-((pyrrolidin-1-yl)methyl)phenyl)methyl)-1H-imidazo[4,5-c]quinolin-4-amine trifluoroacetate

### Step 1: Preparation of 2-methyl sulfonyl-N,N-bis((4-methoxyphenyl)methyl)-1-((4-((pyrrolidin-1-yl)methyl)phenyl)methyl)-1H-imidazo[4,5-c]quinoline-4-amine

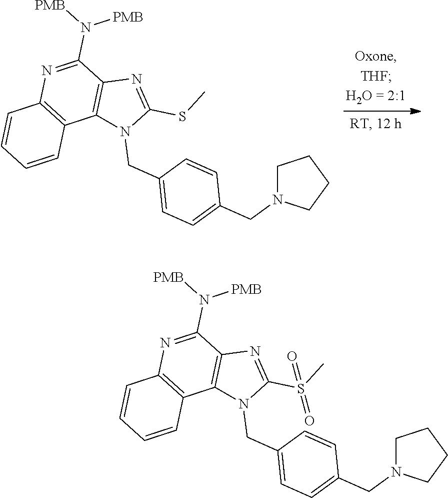

The starting material N,N-bis((4-methoxyphenyl)methyl)-2-(methylthio)-1-((4-((pyrrolidin-1-yl)methyl)phenyl)methyl)-1H-imidazo[4,5-c]quinolin-4-amine (10 g, 15.53 mmol, 1 eq) was dissolved in tetrahydrofuran (100 mL). An aqueous solution (140 mL) of potassium peroxymonosulfonate (38.19 g, 62.13 mmol, 4 eq) was added. The reaction mixture was stirred at room temperature for 12 h. After the reaction was completed, as detected by LC-MS, the reaction mixture was filtered to remove a large amount of potassium peroxomonosulfonate, and concentrated by rotary evaporation to remove tetrahydrofuran. Water (100 mL) was added to the reaction mixture, which was then extracted with ethyl acetate (100 mL×3). The organic phase was concentrated to give a crude product, which was purified by a chromatography column (dichloromethane:methanol=10:1) to give the target product (7 g, 10.36 mmol, purity: 82%). LC-MS (ESI) [M+H]⁺=676.1.

### Step 2: Preparation of 2-methoxy-N,N-bis((4-methoxyphenyl)methyl)-1-((4-((pyrrolidin-1-yl)methyl)phenyl)methyl)-1H-imidazo[4,5-c]quinoline-4-amine

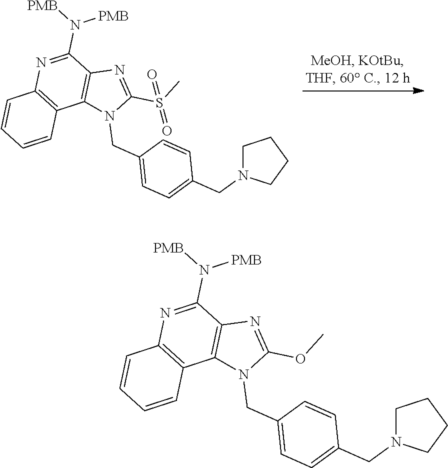

2-methyl sulfonyl-N,N-bis((4-methoxyphenyl)methyl)-1-((4-((pyrrolidin-1-yl)methyl)phenyl)methyl)-1H-imidazo[4,5-c]quinoline-4-amine (300 mg, 0.44 mmol, 1 eq) was dissolved in methanol (6 mL) and tetrahydrofuran (6 mL). Potassium tert-butoxide (199 mg, 1.78 mmol, 4 eq) was added. The mixture was reacted at 60° C. for 12 h. After the reaction was completed, as detected by LC-MS, the reaction mixture was concentrated by rotary evaporation to remove the tetrahydrofuran and methanol, added with water (20 mL) for dilution, and extracted with ethyl acetate (20 mL×3). The organic phases were combined, washed with saturated brine, dried over anhydrous sodium sulfate, and concentrated by rotary evaporation to give a crude product (200 mg, crude product). LC-MS (ESI) [M+H]⁺=628.4.

### Step 3: Preparation of 2-methoxy-1-((4-((pyrrolidin-1-yl)methyl)phenyl)methyl)-1H-imidazo[4,5-c]quinoline-4-amine trifluoroacetate

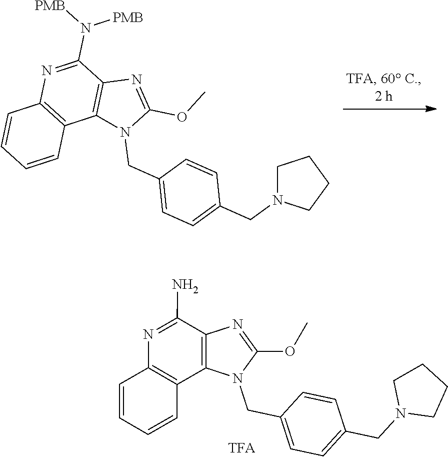

2-methoxy-N,N-bis((4-methoxyphenyl)methyl)-1-((4-((pyrrolidin-1-yl)methyl)phenyl)methyl)-1H-imidazo[4,5-c]quinoline-4-amine (150 mg, 0.24 mmol, 1 eq) was dissolved in trifluoroacetic acid (3 mL). The mixture was stirred at 60° C. for 2 h. After the reaction was completed, as detected by LC-MS, the reaction mixture was concentrated by rotary evaporation to remove the trifluoroacetic acid. The crude product was purified by prep-HPLC (aqueous trifluoroacetic acid solution/MeCN) to give the target compound (11.16 mg, purity: 99%, yield: 12%). LC-MS (ESI) [M+H]⁺=388.3; ¹H NMR (400 MHz, DMSO-d₆): δ 13.67 (s, 1H), 9.82 (s, 1H), 8.90 (s, 2H), 7.90 (d, J=8.5 Hz, 1H), 7.79 (d, J=8.2 Hz, 1H), 7.61 (d, J=7.6 Hz, 1H), 7.48 (d, J=7.1 Hz, 2H), 7.37 (d, J=7.4 Hz, 1H), 7.29 (d, J=7.4 Hz, 2H), 5.77 (s, 2H), 4.30 (s, 2H), 4.26 (s, 3H), 3.05 (s, 4H), 2.00 (s, 2H), 1.82 (s, 2H).

### Test Example 1: Assay on Agonistic Activity for Human-Derived Receptor TLR7

### 1. Experimental Method

HEK-Blue™ hTLR7 cells (InvivoGen, hkb-htlr7) were cultured in a DMEM complete medium (Gibco, 12100) containing 10% FBS (Invitrogen, 10099141), 1% P/S (Invitrogen, 15140122), 10 μg/mL Blasticidin (InvivoGen, ant-bl-1), 100 μg/mL Zeocin (InvivoGen, ant-zn-1), and 100 μg/mL Normocin (InvivoGen, ant-nr-1). The cells were cultured in an incubator at 37° C. with 5% CO₂. When HEK-Blue™ hTLR7 cells reached a growth density of 70%-80%, cell passage was needed.

The agonistic activity for the human-derived receptor TLR7 by HEK-Blue™ hTLR7 cells was assayed by HEK-Blue™ Detection Medium (InvivoGen, hd-det2). First, a bag of HEK-Blue™ Detection Medium powder was dissolved in 50 mL of ultra-pure sterile water for complete dissolution, and then the dissolved medium was filtered into a new 50 mL centrifuge tube through a 0.22 μm filter. Finally, antibiotics were added into the filtered detection medium until the final concentration was 1% for P/S and 100 μg/mL for Normocin. HEK-Blue™ Detection Medium was pre-heated to 37° C. in advance on the day of the assay on the activity for the TLR7 receptor, and the temperature was always maintained at 37° C.

The compound powder was dissolved in 100% DMSO (Sigma, D2650-100ML) to obtain a 10 mM stock solution, and the compound was completely dissolved by shaking with an oscillator. The stock solution of the compound was subjected to a 5-fold dilution to 2 mM with 100% DMSO. This was followed by a 3-fold serial dilution for a total of 8 concentration gradients. Then, each of the concentration gradients was subjected to a 20-fold dilution to 100 μM, 33.3 μM, 11.1 μM, 3.7 μM, 1.23 μM, 0.141 μM, 0.114 μM, and 0.05 μM using HEK-Blue™ Detection Medium. 20 μL of each of the dilutions was added to a 96-well cell culture plate (Corning, 3599).

On the day of the assay on the agonistic activity for the human-detived receptor TLR7, HEK-Blue™ hTLR7 cells has a growth density of 70%-80%, and the cell medium was removed. 1 mL of cell dissociation buffer (Gibco, 13151-014) was added to each 100 mm cell culture dish, and the cells were incubated in an incubator at 37° C. for 5 min. The cells were pipetted using a DMEM cell medium to obtain a single cell suspension, which was then centrifuged at 1000 rpm for 10 min to collect the cell pellet. The cell pellet was resuspended with HEK-Blue™ Detection Medium pre heated to 37° C. The cells were counted and diluted to 220,000 cells/mL. Finally, 180 μL of cell suspension (40,000 cells/well) was added to each well of the 96-well cell culture plate to which the compound had been added, and the cell suspension was mixed with the compound using a pipettor. Final concentrations of the compound were 10 μM, 3.3 μM, 1.1 μM, 0.37 μM, 0.12 μM, 0.04 μM, 0.01 μM, and 0.005 μM. After incubation in an incubator at 37° C. for 16 h, the absorbance was read at 655 nm using an M5e microplate reader (MD, USA).

The EC₅₀ of the compound was calculated using the software GraphPad Prism (GraphPad Software, USA). The background reading of the detection culture was subtracted from the absorbance readings at 655 nm of all wells of the 96-well plate, the ratio of each well to the negative control well (the well without the compound) was calculated, and then the percentage of the ratio of each well to the positive control well (the well treated with 10 μM positive compound), i.e., the percentage of activity, was calculated. Finally, the sigmoidal dose-response (variable slope) curve between the percentage of activity and the concentration of the compound was calculated using the software GraphPad Prism to calculate the EC₅₀ of the compound.

### 2. Experimental Results

The EC₅₀ results for the compounds of the present disclosure for human-detived TLR7 are shown in the table below, wherein B represents that 50 nm≤EC50<100 nm, and A represents that EC50<50 nm.

| Example | EC50 | Example | EC50 |
|---|---|---|---|
| compound | (nM) | compound | (nM) |
| Example 40 | A | Example 42 | A |
| Example 43 | A | Example 44 | A |
| Example 45 | A | Example 46 | A |
| Example 47 | A | Example 48 | A |
| Example 49 | A | Example 50 | A |
| Example 66 | B | Example 76 | B |
| Example 78 | A | Example 113 | B |
| Example 155 | A | Example 156 | A |
| Example 157 | A | Example 159 | A |
| Example 161 | B | Example 163 | A |
| Example 165 | B | Example 166 | A |
| Example 177 | B | Example 178 | B |
| Example 179 | A | Example 181 | A |
| Example 188 | B | Example 189 | B |
| Example 190 | B | Example 191 | A |
| Example 196 | B | Example 199 | A |
| Example 206 | B | Example 207 | B |
| Example 209 | B | Example 210 | B |
| Example 211 | B | Example 212 | A |
| Example 213 | B | Example 214 | B |
| Example 215 | A | Example 220 | B |
| Example 223 | B | Example 233 | A |
| Example 238 | B | Example 239 | B |
| Example 240 | A | Example 241 | B |
| Example 244 | B | Example 245 | B |
| Example 246 | B | Example 247 | B |
| Example 248 | B | Example 252 | A |

Conclusion: the compounds of the present disclosure have a significant activation effect on human-detived TLR7.

### Test Example 2: Selective Investigation of Agonistic Activity for Human-Detived Receptor TLR7/8

### 1. Experimental Method

The assay on the agonistic activity for human-detived receptor TLR7 was performed as that in Test Example 1;
the method for assaying the agonistic activity for the human-detived receptor TLR8 was as follows: HEK-Blue™ hTLR8 cells (InvivoGen, hkb-htlr8) were cultured in a DMEM complete medium (Gibco, 11965-092) containing 10% FBS (Invitrogen, 10099-141c), 1% P/S (Invitrogen, 15140-122), 10 μg/mL Blasticidin (InvivoGen, R210-01), 100 μg/mL Zeocin (InvivoGen, ant-zn), and 100 μg/mL Normocin (InvivoGen, Ant-nr-2). The cells were cultured in an incubator at 37° C. with 5% CO₂. When HEK-Blue™ hTLR8 cells reached a growth density of 70%-80%, cell passage was needed.

The agonistic activity for the human-detived receptor TLR8 by HEK-Blue™ hTLR8 cells was assayed by HEK-Blue™ Detection Medium (InvivoGen, hb-det3). First, a bag of HEK-Blue™ Detection Medium powder was completely dissolved, and then filtered through a 0.22 μm filter. Antibiotics were then added until the final concentration was 1% P/S and 100 μg/mL Normocin. HEK-Blue™ Detection Medium was pre-heated to 37° C. in advance on the day of the assay on the activity for the TLR8 receptor, and the temperature was always maintained at 37° C.

The compound powder was dissolved in 100% DMSO (Sigma, D8418-1L) to obtain a 10 mM stock solution, and the compound was completely dissolved by shaking with an oscillator for later use.

The cell culture medium was removed from HEK-Blue™ hTLR8 cells with a growth density of 70-80%. Then, the cells were washed 2-3 times with PBS (Gibco, 10010-031). The cells were digested and then pipetted with a DMEM cell culture medium to a single cell suspension, which was then centrifuged at 1000 rpm for 10 min to collect the cell pellet. The cell pellet was resuspended with HEK-Blue™ Detection Medium pre-heated to 37° C. The cells were counted, diluted to a plating density, and transferred to a 384-well cell culture plate (Corning, 3764). The cell suspension was mixed with the compound using Echo (LABCYTE, 550) to make the final concentrations of 10 μM, 3.3 μM, 1.1 μM, 0.37 μM, 0.12 μM, 0.04 μM, 0.01 μM, and 0.005 μM for the compound, and the content of 0.33% for DMSO. After incubation in an incubator at 37° C. for 16 h, the absorbance was read at 620 nm using a microplate reader (PerkinElmer, VICTOR Nivo).

The EC₅₀ of the compound was calculated using the GraphPad Prism 8 software (GraphPad Software, USA). The reading of the negative control well (the well without the compound) was subtracted from the absorbance reading at 620 nm of the compound, and was divided by the ratio of the difference between the positive control (1 μM positive compound) and the negative control well, so that the percentage of the ratio of each well to the ratio of the positive control well (the well treated with 10 μM positive compound), i.e., the percentage of activity, was calculated. Finally, the sigmoidal dose-response (variable slope) curve between the percentage of activity and the concentration of the compound was calculated using the software GraphPad Prism to calculate the EC₅₀ of the compound.

### 2. Experimental Results:

The EC₅₀ results for the compounds of the present disclosure for human-detived TLR7/8 are shown in the table below.

| Example | Compound structure | hTLR7 EC₅₀ (nM) | hTLR8 EC₅₀ (nM) |
|---|---|---|---|
| Example 40 | 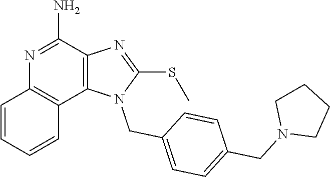 [SMILES: CSc1nc2c(N)nc3ccccc3c2n1Cc1ccc(CN2CCCC2)cc1] | 55 | >10000.0 |
| Example 199 | 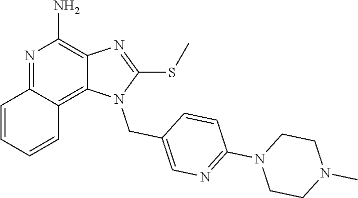 [SMILES: CSc1nc2c(N)nc3ccccc3c2n1Cc1ccc(N2CCN(C)CC2)nc1] | 43.6 | >10000.0 |
| Example 240 | 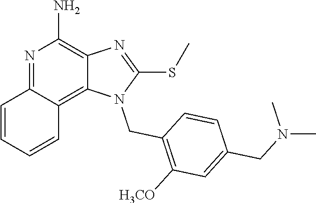 [SMILES: COc1cc(CN(C)C)ccc1Cn1c(SC)nc2c(N)nc3ccccc3c21] | 28.4 | >10000.0 |
| Comparative Example 1 | | >10000.0 | >10000.0 |

Conclusion: the compounds of the present disclosure have significant activation selectivity for human-detived TLR7.

1. A compound represented by formula (I), and a stereoisomer, an optical isomer, a pharmaceutically acceptable salt, a prodrug, and a solvate thereof, 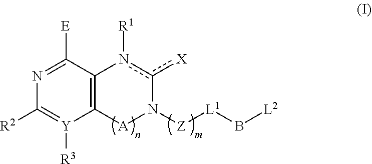
wherein,
E is selected from hydrogen, amino, and halogen;
R¹ is selected from hydrogen, hydroxyl, amino, C₁₋₆ alkyl, C₃₋₆ cycloalkyl, 4- to 6-membered heterocyclyl, aryl, and 5- to 6-membered heteroaryl, or R¹ is absent;
the compound fragment 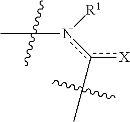
is selected from 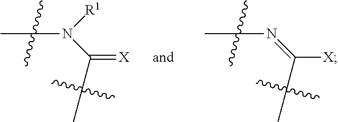
when the compound fragment is selected from 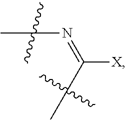
R¹ is absent;
when 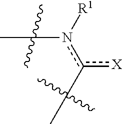
is selected from 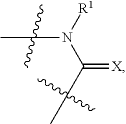
X is absent, or X is selected from O, S, C(R⁸)(R⁹), and N(R⁸); wherein, R⁸ or R⁹, at each occurrence, is independently selected from hydrogen, halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl;
when 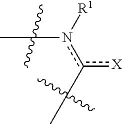
is selected from 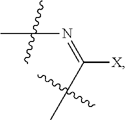
X is selected from hydrogen, acylamino, formyl, acetyl, carboxyl, cyano, —O(R²⁰), —S(O)_{f}(R²⁰), optionally substituted C₁₋₆ alkyl, optionally substituted —OC₁₋₆ alkyl, optionally substituted —SC₁₋₆ alkyl, optionally substituted —OC₃₋₆ cycloalkyl, optionally substituted —SC₃₋₆ cycloalkyl, optionally substituted —COOC₁₋₆ alkyl, optionally substituted —SC₁₋₆ alkyl-COOC₁₋₆ alkyl, optionally substituted —OC₁₋₆ alkyl-COOC₁₋₆ alkyl, and optionally substituted —SC₁₋₆ alkyl-OC₁₋₆ alkyl, the “optionally substituted” means being unsubstituted or being substituted with one or more substituents selected from hydroxyl, carboxyl, halogen, cyano, amino, acylamino, C₁₋₆ alkyl, C₂₋₆ alkenyl, C₃₋₆ cycloalkyl, methoxy, and methylthio; wherein, R²⁰, at each occurrence, is independently selected from hydrogen, halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl; f is selected from 0, 1, and 2;
R² is selected from hydrogen, halogen, hydroxyl, sulfhydryl, amino, cyano, R⁴, —O(R⁴), —S(R⁴), —N(R⁴)(R⁵), 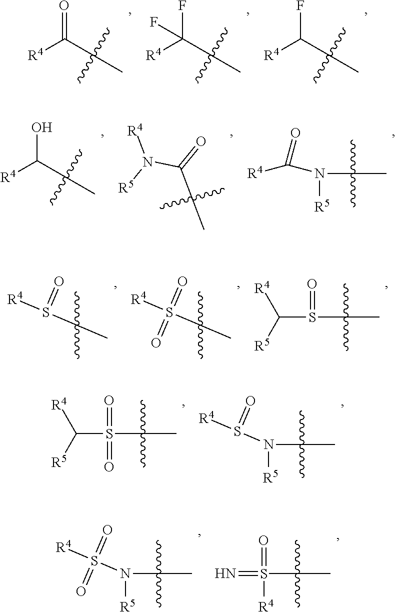
—PO(R⁴), —N(R⁵)PO(R⁴), and —PON(R⁵)(R⁴); R⁴ or R⁵, at each occurrence, is independently selected from C₁₋₆ alkyl, C₁₋₆ haloalkyl, C₂₋₆ alkenyl, C₂₋₆ alkynyl, C₃₋₆ cycloalkyl, 4- to 6-membered heterocyclyl, aryl, and 5- to 6-membered heteroaryl;
A, at each occurrence, is independently selected from —CO— and —C(R⁶)(R⁷)—; wherein, R⁶ or R⁷, at each occurrence, is independently selected from hydrogen, halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl; n is selected from 0, 1, and 2;
Y is selected from C and N;
when Y is selected from N, R³ is absent;
when Y is selected from C, R³ is selected from hydrogen, halogen, hydroxyl, sulfhydryl, amino, cyano, C₁₋₆ alkyl, C₃₋₆ cycloalkyl, 4- to 6-membered heterocyclyl, aryl, and 5- to 6-membered heteroaryl; alternatively, R² and R³, together with the atom connected thereto, form an H ring, the H ring being an optionally substituted 4- to 10-membered ring selected from C₄₋₈ cycloalkene, 4- to 6-membered heterocyclyl ring, phenyl ring, and 5- to 6-membered heteroaryl ring; the “optionally substituted” refers to being unsubstituted or being substituted with one or more groups selected from halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, 5- to 6-membered heteroaryl, —NH(C₁₋₆ alkyl), —N(C₁₋₆ alkyl)(C₁₋₆ alkyl), —OC₁₋₆ alkyl, —SC₁₋₆ alkyl, —SOC₁₋₆ alkyl, and —SO(NH)(C₁₋₆ alkyl);
Z, at each occurrence, is independently selected from —O—, —S—, —C(R¹⁰)(R¹¹)—, —CO—, —CS—, —CO₂—, —CON(R¹⁰)—, —SON(R¹⁰)—, —SO₂N(R¹⁰)—, —N(R¹⁰)—, —SO—, —SO₂—, and —P(O)(R¹⁰)—; wherein, R¹⁰ or R¹¹, at each occurrence, is independently selected from hydrogen, halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl; m is selected from 0, 1, 2, and 3;
B is selected from -(chemical bond), —O—, —S—, —N(R¹²)—, —CO—, —SO—, —SO₂—, —(CH₂)ₚN(R¹²)—, —N(R¹²)(CH₂)ₚ—, —S(O)N(R¹²)—, —S(O)₂N(R¹²)—, —N(R¹²)SO—, —N(R¹²)S(O)₂—, —C(O)N(R¹²)—, —N(R¹²)C(O)—, —C(R¹²)(R¹³)—, and —C(R¹²)(R¹³)—C(R¹²)(R¹³)—; wherein, p=0, 1, 2, or 3, and R¹² or R¹³, at each occurrence, is independently selected from hydrogen, deuterium, halogen, hydroxyl, sulfhydryl, amino, cyano, optionally substituted C₁₋₆ alkyl, optionally substituted C₂₋₆ alkenyl, optionally substituted C₂₋₆ alkynyl, optionally substituted C₃₋₆ cycloalkyl, optionally substituted 4- to 6-membered heterocyclyl, optionally substituted phenyl, and optionally substituted 5- to 6-membered heteroaryl; the “optionally substituted” refers to being unsubstituted or being substituted with one or more groups selected from halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl;
L¹ is selected from optionally substituted C₁₋₁₂ alkyl, optionally substituted C₂₋₁₂ alkenyl, optionally substituted C₂₋₁₂ alkynyl, optionally substituted C₆₋₁₀ aryl, optionally substituted 5- to 10-membered heteroaryl, optionally substituted C₃₋₈ cycloalkyl, and optionally substituted 4- to 10-membered heterocyclyl; the “optionally substituted” refers to being unsubstituted or being substituted with one or more R¹⁴; wherein, R¹⁴, at each occurrence, is independently selected from hydrogen, halogen, hydroxyl, sulfhydryl, amino, nitro, cyano, —R¹⁵, —OR¹⁵, —SR¹⁵, SO(R¹⁵), —SO₂(R¹⁵), —COR¹⁵, —COOR¹⁵, —N(R¹⁵)(R¹⁶), —CONHR¹⁵, —CON(R¹⁵)(R¹⁶), —SONH(R¹⁵), —SON(R¹⁵)(R¹⁶), SO₂NH(R¹⁵), and —SO₂N(R¹⁵)(R¹⁶); wherein, R¹⁵ or R¹⁶, at each occurrence, is independently selected from C₁₋₆ alkyl, C₂₋₆ alkenyl, C₂₋₆ alkynyl, C₃₋₆ cycloalkyl, 4- to 6-membered heterocyclyl, aryl, and 5- to 6-membered heteroaryl, which may be optionally substituted with one or more of hydrogen, halogen, hydroxyl, sulfhydryl, amino, cyano, C₁₋₆ alkyl, C₃₋₆ cycloalkyl, 4- to 6-membered heterocyclyl, aryl, and 5- to 6-membered heteroaryl;
L² is absent, or L² is selected from optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted 4- to 10-membered heterocyclyl, optionally substituted C₆₋₁₀ aryl, optionally substituted 5- to 10-membered heteroaryl, optionally substituted —NHC₁₋₆ alkyl, and optionally substituted —N(C₁₋₆ alkyl)₂; the “optionally substituted” refers to being unsubstituted or being substituted with one or more R¹⁷, wherein, R¹⁷, at each occurrence, is independently selected from hydrogen, halogen, hydroxyl, sulfhydryl, nitro, amino, cyano, oxo, —R¹⁸, —OR¹⁸, —SR¹⁸, —SO(R¹⁸), —SO₂(R¹⁸), —COOR¹⁸, —COR¹⁸, —NH(R¹⁸), —N(R¹⁸)(R¹⁹), —CONHR¹⁹, —CON(R¹⁸)(R¹⁹), —SONH(R¹⁸), —SON(R¹⁸)(R¹⁹), SO₂NH(R¹⁸), —SO₂N(R¹⁸)(R¹⁹), —C₀₋₃ alkyl-N(R¹⁸)(R¹⁹), —COC₁₋₃ alkyl-O(R¹⁸), —COC₁₋₃ alkyl-NH₂, and —C₁₋₃ alkyl-N(R¹⁸)(R¹⁹); wherein, the oxo means that two H at the same substitution site are substituted with the same O to form a divalent substituent ═O, and R¹⁸ or R¹⁹, at each occurrence, is independently selected from hydrogen, C₁₋₆ alkyl, C₁₋₆ haloalkyl, C₂₋₆ alkenyl, C₂₋₆ alkynyl, C₃₋₆ cycloalkyl, 4- to 6-membered heterocyclyl, aryl, and 5- to 6-membered heteroaryl;
unless otherwise stated, the heteroatoms in the heteroaryl and heterocyclyl described above are independently selected from O, N, and S, and the number of the heteroatoms is 1, 2, 3, or 4.

2-9. (canceled)

10. The compound, and the stereoisomer, the optical isomer, the pharmaceutically acceptable salt, the prodrug, and the solvate thereof according to claim 1, wherein, 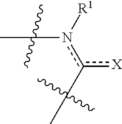
is selected from 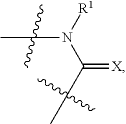
and X is selected from O, S, CH₂, CHF, CHOH, CF₂, NH, and NCH₃, preferably O, S, and CH₂, more preferably O.

11. The compound, and the stereoisomer, the optical isomer, the pharmaceutically acceptable salt, the prodrug, and the solvate thereof according to claim 1, wherein, 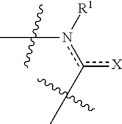
is selected from 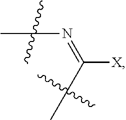
and X is selected from O(R²⁰) and S(O)_{f}(R²⁰);
wherein, R²⁰, at each occurrence, is independently selected from hydrogen, halogen, hydroxyl, sulfhydryl, amino, cyano, C₁₋₄ alkyl, C₁₋₃ alkenyl, C₁₋₃ alkynyl, C₃₋₆ cycloalkyl, 4- to 6-membered heterocyclyl, aryl, and 5- to 6-membered heteroaryl, and preferably, R²⁰, at each occurrence, is independently selected from hydrogen, halogen, hydroxyl, methyl, ethyl, n-propyl, n-butyl, isopropyl, cyclopropyl, cyclobutyl, and cyclopentyl; f is selected from 0 and 1.

12-13. (canceled)

14. The compound, and the stereoisomer, the optical isomer, the pharmaceutically acceptable salt, the prodrug, and the solvate thereof according to claim 1,
wherein, Y is selected from C, and R³ is selected from hydrogen, halogen, hydroxyl, sulfhydryl, amino, cyano, C₁₋₆ alkyl, C₃₋₆ cycloalkyl, 4- to 6-membered heterocyclyl, aryl, and 5- to 6-membered heteroaryl, preferably hydrogen, halogen, hydroxyl, sulfhydryl, amino, cyano, methyl, ethyl, n-propyl, isopropyl, cyclopropyl, C₅₋₆ cycloalkyl, 5- to 6-membered heterocycloalkyl, C₆ aryl, and 5- to 6-membered heteroaryl.

15. The compound, and the stereoisomer, the optical isomer, the pharmaceutically acceptable salt, the prodrug, and the solvate thereof according to claim 1,
wherein, when Y is selected from C, R² and R³ may be connected to form an H ring, the H ring being an optionally substituted 4- to 10-membered ring selected from C₄₋₈ cycloalkene, 4- to 6-membered heterocyclyl ring, phenyl ring, and 5- to 6-membered heteroaryl ring; the “optionally substituted” refers to being unsubstituted or being substituted with one or more groups independently selected from halogen, hydroxyl, sulfhydryl, amino, cyano, C₁₋₆ alkyl, C₁₋₆ alkenyl, C₁₋₆ alkynyl, C₃₋₆ cycloalkyl, 4- to 6-membered heterocyclyl, aryl, and 5- to 6-membered heteroaryl; preferably, the “optionally substituted” refers to being unsubstituted or being substituted with one or more groups independently selected from halogen, hydroxyl, sulfhydryl, amino, cyano, methyl, ethyl, n-propyl, isopropyl, cyclopropyl, n-butyl, isobutyl, cyclobutyl, ethenyl, ethynyl, 5- to 6-membered heterocyclyl, C₆ aryl, and 5- to 6-membered heteroaryl.

16. The compound, and the stereoisomer, the optical isomer, the pharmaceutically acceptable salt, the prodrug, and the solvate thereof according to claim 1,
wherein, Z, at each occurrence, is independently selected from —O—, —S—, —C(R¹⁰)(R¹¹)—, —CO—, —CS—, —CO₂—, —CON(R¹⁰)—, —SON(R¹⁰)—, —SO₂N(R¹⁰)—, —N(R¹⁰)—, —SO—, and —SO₂—, preferably —C(R¹⁰)(R¹¹)—, —CO—, —CON(R¹⁰)—, —N(R¹⁰)—, and —SO₂—, more preferably —C(R¹⁰)(R¹¹)—, —CO—, —N(R¹⁰)—, and —SO₂—; wherein, R¹⁰ or R¹¹, at each occurrence, is independently selected from hydrogen, halogen, hydroxyl, sulfhydryl, amino, cyano, C₁₋₆ alkyl, C₁₋₆ alkenyl, C₁₋₆ alkynyl, C₃₋₆ cycloalkyl, 4- to 6-membered heterocyclyl, aryl, and 5- to 6-membered heteroaryl, preferably hydrogen, halogen, hydroxyl, sulfhydryl, amino, cyano, C₁₋₄ alkyl, C₂₋₄ alkenyl, C₂₋₄ alkynyl, C₃₋₆ cycloalkyl, 4- to 6-membered heterocyclyl, C₆ aryl, and 5- to 6-membered heteroaryl, more preferably hydrogen, halogen, hydroxyl, sulfhydryl, amino, cyano, methyl, ethyl, n-propyl, isopropyl, and cyclopropyl.

17. (canceled)

18. The compound, and the stereoisomer, the optical isomer, the pharmaceutically acceptable salt, the prodrug, and the solvate thereof according to claim 1,
wherein, B is selected from -(chemical bond), —O—, —S—, —N(R¹²)—, —CO—, —SO—, —SO₂—, —(CH₂)ₚN(R¹²)—, —N(R¹²)(CH₂)ₚ—, —S(O)N(R¹²)—, —S(O)₂N(R¹²)—, —N(R¹²)SO—, —N(R¹²)S(O)₂—, —C(O)N(R¹²)—, —N(R¹²)C(O)—, and —C(R¹²)(R¹³)—; wherein, p=0, 1, 2, or 3; R¹² or R¹³, at each occurrence, is independently selected from hydrogen, halogen, hydroxyl, sulfhydryl, amino, cyano, C₁₋₆ alkyl, C₂₋₆ alkenyl, C₂₋₆ alkynyl, C₃₋₆ cycloalkyl, 4- to 6-membered heterocyclyl, C₆ aryl, and 5- to 6-membered heteroaryl;
preferably, B is selected from -(chemical bond), —O—, —S—, —N(R¹²)—, —CO—, —SO—, —SO₂—, —(CH₂)ₚN(R¹²)—, —N(R¹²)(CH₂)ₚ—, —S(O)N(R¹²)—, —S(O)₂N(R¹²)—, —N(R¹²)SO—, —N(R¹²)S(O)₂—, —C(O)N(R¹²)—, —N(R¹²)C(O)—, and —C(R¹²)(R¹³)—, wherein, p=0, 1, 2, or 3; R¹² or R¹³, at each occurrence, is independently selected from hydrogen, halogen, hydroxyl, C₁₋₆ alkyl, C₃₋₆ cycloalkyl, and 4- to 6-membered heterocyclyl.

19-26. (canceled)

27. A compound represented by formula (IV), and a stereoisomer, an optical isomer, a pharmaceutically acceptable salt, a prodrug, and a solvate thereof: 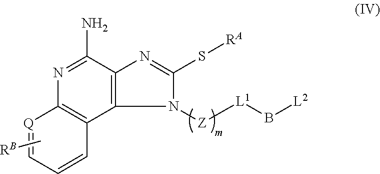
wherein, R^{A} is selected from optionally substituted C₁₋₆ alkyl, optionally substituted C₃₋₆ cycloalkyl, and optionally substituted —CH₂COOC₁₋₃ alkyl, the “optionally substituted” means being unsubstituted or being substituted with one or more substituents selected from hydroxyl, amino, nitro, carboxyl, cyano, acylamino, halogen, C₁₋₆ alkyl, C₃₋₆ cycloalkyl, C₂₋₆ alkenyl, C₂₋₆ alkynyl, and C₁₋₆ alkoxy;
R^{B} is selected from hydrogen, C₁₋₆ alkyl, hydroxyl, amino, carboxyl, cyano, nitro, halogen, C₁₋₆ alkoxy, and C₁₋₆ hydroxyalkyl;
Q is selected from C and N;
Z, at each occurrence, is independently selected from —O—, —S—, —C(R¹⁰)(R¹¹)—, —CO—, and —CS—;
wherein, R¹⁰ or R¹¹, at each occurrence, is independently selected from hydrogen, halogen, hydroxyl, sulfhydryl, amino, cyano, C₁₋₆ alkyl, C₂₋₆ alkenyl, and C₂₋₆ alkynyl; m is selected from 0, 1, and 2;
B is selected from -(chemical bond), —O—, —S—, —N(R¹²)—, —CO—, and —C(R¹²)(R¹³)—; wherein, R¹² or R¹³, at each occurrence, is independently selected from hydrogen, deuterium, halogen, hydroxyl, sulfhydryl, amino, cyano, and C₁₋₆ alkyl;
L¹ is selected from optionally substituted C₁₋₁₂ alkyl, optionally substituted C₂₋₁₂ alkenyl, optionally substituted C₂₋₁₂ alkynyl, optionally substituted C₆₋₁₀ aryl, optionally substituted 5- to 10-membered heteroaryl, and optionally substituted 4- to 10-membered heterocyclyl; the “optionally substituted” refers to being unsubstituted or being substituted with one or more R¹⁴;
wherein, R¹⁴, at each occurrence, is independently selected from halogen, hydroxyl, sulfhydryl, amino, nitro, cyano, C₁₋₆ alkyl, C₁₋₆ alkoxy, and C₃₋₆ cycloalkyl;
L² is absent, or L² is selected from optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted 4- to 10-membered heterocyclyl, optionally substituted C₆₋₁₀ aryl, optionally substituted 5- to 10-membered heteroaryl, optionally substituted —NHC₃₋₆ cycloalkyl, optionally substituted —NHC₁₋₆ alkyl, and optionally substituted —N(C₁₋₆ alkyl)₂, and preferably, L² is selected from absence or L² is selected from optionally substituted C₁₋₁₂ alkyl, optionally substituted C₂₋₁₂ alkenyl, optionally substituted C₂₋₁₂ alkynyl, optionally substituted C₃₋₁₂ cycloalkyl, optionally substituted 4- to 10-membered heterocyclyl, optionally substituted C₆₋₁₀ aryl, optionally substituted 5- to 10-membered heteroaryl, optionally substituted —NHC₁₋₆ alkyl, and optionally substituted —N(C₁₋₆ alkyl)₂; the “optionally substituted” refers to being unsubstituted or being substituted with one or more R¹⁷; wherein, R¹⁷, at each occurrence, is independently selected from hydrogen, halogen, hydroxyl, sulfhydryl, nitro, amino, cyano, oxo, —R¹⁸, —OR¹⁸, —SR¹⁸, —SO(R¹⁸), SO₂(R¹⁸), —COOR¹⁸, —COR¹⁸, —NH(R¹⁸), —N(R¹⁸)(R¹⁹), —CONHR¹⁹, —CON(R¹⁸)(R¹⁹), —SONH(R¹⁸), —SON(R¹⁸)(R¹⁹), SO₂NH(R¹⁸), —SO₂N(R¹⁸)(R¹⁹), —C₀₋₃ alkyl-N(R¹⁸)(R¹⁹), —COC₁₋₃ alkyl-O(R¹⁸), and —C₁₋₃ alkyl-N(R¹⁸)(R¹⁹); wherein, the oxo means that two H at the same substitution site are substituted with the same O to form a divalent substituent ═O, and R¹⁸ or R¹⁹, at each occurrence, is independently selected from hydrogen, C₁₋₆ alkyl, C₁₋₆ haloalkyl, C₂₋₆ alkenyl, C₂₋₆ alkynyl, C₃₋₆ cycloalkyl, 4- to 6-membered heterocyclyl, aryl, and 5- to 6-membered heteroaryl;
the heteroatoms in the heterocyclyl and the heteroaryl are selected from N, O, and S, and the number of the heteroatoms is 1, 2, or 3.

28. The compound, and the stereoisomer, the optical isomer, the pharmaceutically acceptable salt, the prodrug, and the solvate thereof according to claim 27,
wherein, R^{A} is selected from optionally substituted C₁₋₆ alkyl, optionally substituted C₃₋₆ cycloalkyl, and optionally substituted —CH₂COOC₁₋₃ alkyl, the “optionally substituted” means being unsubstituted or being substituted with one or more substituents selected from hydroxyl, amino, carboxyl, cyano, acylamino, halogen, methoxy, C₁₋₆ alkyl, C₃₋₆ cycloalkyl, and C₂₋₆ alkenyl;
preferably, R^{A} is selected from methyl, ethyl, n-propyl, isopropyl, n-butyl, isobutyl, tert-butyl, n-pentyl, 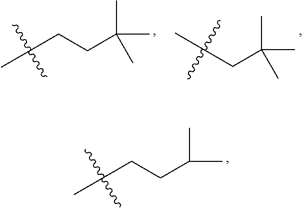
cyclopropyl, cyclobutyl, cyclopentyl, —CH₂COOCH₃, and —CH₂COOCH₂CH₃, the R^{A} being optionally substituted with one or more substituents selected from hydroxyl, amino, carboxyl, cyano, acylamino, halogen, methoxy, methyl, ethyl, ethenyl, cyclopropyl, cyclobutyl, and cyclopentyl;
more preferably, R^{A} is selected from methyl, ethyl, n-propyl, isopropyl, n-butyl, isobutyl, tert-butyl, n-pentyl, 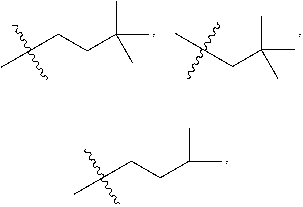
cyclopropyl, cyclobutyl, cyclopentyl, —CH₂COOCH₃, —CH₂COOCH₂CH₃, monofluoromethyl, difluoromethyl, trifluoromethyl, monofluoroethyl, difluoroethyl, trifluoroethyl, monofluoro-n-propyl, difluoro-n-propyl, trifluoro-n-propyl, 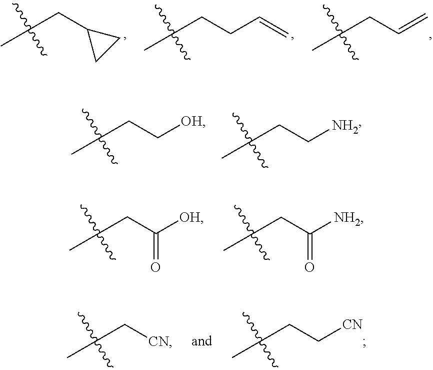
further preferably, R^{A} is selected from methyl, ethyl, n-propyl, n-butyl 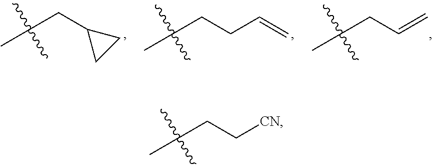
—CH₂CH₂F, —CH₂OCH₃, —CH₂CH(F)₂, and —CH₂CH₂C(F)₃;
still further preferably, R^{A} is selected from methyl.

29. The compound, and the stereoisomer, the optical isomer, the pharmaceutically acceptable salt, the prodrug, and the solvate thereof according to claim 27,
wherein, R^{B} is selected from hydrogen, C₁₋₆ alkyl, halogen, and C₁₋₆ alkoxy;
preferably, R^{B} is selected from hydrogen, methyl, ethyl, F, Cl, Br, methoxy, and ethoxy;
more preferably, R^{B} is selected from hydrogen.

30. The compound, and the stereoisomer, the optical isomer, the pharmaceutically acceptable salt, the prodrug, and the solvate thereof according to claim 27,
wherein, Q is selected from C.

31. The compound, and the stereoisomer, the optical isomer, the pharmaceutically acceptable salt, the prodrug, and the solvate thereof according to claim 27,
wherein, Z, at each occurrence, is independently selected from —O—, —S—, and —C(R¹⁰)(R¹¹)—;
wherein, R¹⁰ or R¹¹, at each occurrence, is independently selected from hydrogen, halogen, hydroxyl, sulfhydryl, amino, cyano, methyl, and ethyl; m is selected from 0, 1, and 2;
preferably, Z, at each occurrence, is independently selected from —C(R¹⁰)(R¹¹)—; wherein, R¹⁰ or R¹¹, at each occurrence, is independently selected from hydrogen, halogen, hydroxyl, sulfhydryl, amino, cyano, methyl, and ethyl; m is selected from 1 and 2;
more preferably, Z, at each occurrence, is independently selected from —C(R¹⁰)(R¹¹)—; wherein, R¹⁰ or R¹¹, at each occurrence, is independently selected from hydrogen and methyl; m is selected from 1 and 2;
further preferably, Z, at each occurrence, is independently selected from —CH₂— and —CH(CH₃)—; m is selected from 1;
still further preferably, Z, at each occurrence, is independently selected from —CH₂—; m is selected from 1.

32. The compound, and the stereoisomer, the optical isomer, the pharmaceutically acceptable salt, the prodrug, and the solvate thereof according to claim 27,
wherein, B is selected from -(chemical bond), —O—, —S—, —CO—, and —C(R¹²)(R¹³)—; wherein, R¹² or R¹³, at each occurrence, is independently selected from hydrogen, deuterium, halogen, hydroxyl, sulfhydryl, amino, cyano, and C₁₋₆ alkyl;
preferably, B is selected from -(chemical bond), —O—, and —C(R¹²)(R¹³)—; wherein, R¹² or R¹³, at each occurrence, is independently selected from hydrogen, methyl, and ethyl;
more preferably, B is selected from -(chemical bond), —O—, and —CH₂—;
further preferably, B is selected from —CH₂—.

33. The compound, and the stereoisomer, the optical isomer, the pharmaceutically acceptable salt, the prodrug, and the solvate thereof according to claim 27,
wherein, L¹ is selected from optionally substituted C₁₋₆ alkyl, optionally substituted C₆₋₁₀ aryl, optionally substituted 5- to 10-membered heteroaryl, and optionally substituted 4- to 10-membered heterocyclyl; the “optionally substituted” refers to being unsubstituted or being substituted with one or more R¹⁴; wherein, R¹⁴, at each occurrence, is independently selected from halogen, hydroxyl, sulfhydryl, amino, nitro, cyano, C₁₋₃ alkyl, and C₁₋₃ alkoxy;
preferably, L¹ is selected from optionally substituted C₁₋₆ alkyl, optionally substituted C₆₋₁₀ aryl, optionally substituted 5- to 10-membered heteroaryl, and optionally substituted 5- to 10-membered heterocyclyl; the “optionally substituted” refers to being unsubstituted or being substituted with one or more R¹⁴; wherein, R¹⁴, at each occurrence, is independently selected from halogen, C₁₋₃ alkyl, and C₁₋₃ alkoxy;
more preferably, L¹ is selected from optionally substituted C₁₋₆ alkyl, optionally substituted phenyl, optionally substituted 5-membered monocyclic heteroaryl, optionally substituted 6-membered monocyclic heteroaryl, optionally substituted 6-membered and 5-membered fused heteroaryl, optionally substituted 5-membered and 6-membered fused heteroaryl, optionally substituted 5-membered monocyclic heterocyclyl, optionally substituted 6-membered monocyclic heterocyclyl, optionally substituted 6-membered and 5-membered fused heterocyclyl, and optionally substituted 5-membered and 6-membered fused heterocyclyl; the “optionally substituted” refers to being unsubstituted or being substituted with one or more R¹⁴; wherein, R¹⁴ at each occurrence, is independently selected from halogen, methyl, ethyl, methoxy, and ethoxy;
further preferably, L¹ is selected from optionally substituted C₁₋₆ alkyl, optionally substituted phenyl, optionally substituted pyridinyl, and optionally substituted thienyl; the “optionally substituted” refers to being unsubstituted or being substituted with one or more R¹⁴; wherein, R¹⁴ at each occurrence, is independently selected from F, Cl, Br, methyl, and methoxy;
still further preferably, L¹ is selected from phenyl, pyridinyl, and thienyl, and is optionally substituted with F, Cl, methyl, and methoxy;
yet still further preferably, L¹ is selected from phenyl.

34. The compound, and the stereoisomer, the optical isomer, the pharmaceutically acceptable salt, the prodrug, and the solvate thereof according to claim 27,
wherein, L² is absent, or L² is selected from optionally substituted C₁₋₆ alkyl, optionally substituted C₃₋₁₀ cycloalkyl, optionally substituted 4- to 10-membered heterocyclyl, optionally substituted C₆₋₁₀ aryl, and optionally substituted 5- to 10-membered heteroaryl; the “optionally substituted” refers to being unsubstituted or being substituted with one or more R¹⁷; wherein, R¹⁷, at each occurrence, is independently selected from hydrogen, halogen, hydroxyl, sulfhydryl, nitro, amino, cyano, oxo, —R¹⁸, —OR¹⁸, —COR¹⁸, —NH(R¹⁸), —N(R¹⁸)(R¹⁹), and —C₀₋₃ alkyl-N(R¹⁸)(R¹⁹); wherein, the oxo means that two H at the same substitution site are substituted with the same O to form a divalent substituent ═O, and R¹⁸ or R¹⁹, at each occurrence, is independently selected from hydrogen, C₁₋₆ alkyl, and C₃₋₆ cycloalkyl;
preferably, L² is selected from optionally substituted C₁₋₆ alkyl, optionally substituted cyclopropyl, optionally substituted cyclobutyl, optionally substituted cyclopentyl, optionally substituted cyclohexyl, optionally substituted 4-membered monocyclic heterocycloalkyl, optionally substituted 5-membered monocyclic heterocycloalkyl, optionally substituted 6-membered monocyclic heterocycloalkyl, optionally substituted 4-membered and 4-membered fused heterocycloalkyl, optionally substituted 4-membered and 5-membered fused heterocycloalkyl, optionally substituted 5-membered and 4-membered fused heterocycloalkyl, optionally substituted 5-membered and 5-membered fused heterocycloalkyl, optionally substituted 5-membered and 6-membered fused heterocycloalkyl, optionally substituted 6-membered and 5-membered fused heterocycloalkyl, optionally substituted 5-membered monocyclic heteroaryl, and optionally substituted 6-membered monocyclic heteroaryl; the “optionally substituted” refers to being unsubstituted or being substituted with one or more R¹⁷;
wherein, R¹⁷, at each occurrence, is independently selected from hydrogen, halogen, hydroxyl, amino, —R¹⁸, —OR¹⁸, —N(R¹⁸)(R¹⁹), and —C₀₋₃ alkyl-N(R¹⁸)(R¹⁹); wherein, R¹⁸ or R¹⁹, at each occurrence, is independently selected from hydrogen, C₁₋₆ alkyl, and C₃₋₆ cycloalkyl;
more preferably, L² is selected from optionally substituted C₁₋₆ alkyl, optionally substituted 4-membered monocyclic heterocycloalkyl, optionally substituted 5-membered monocyclic heterocycloalkyl, optionally substituted 6-membered monocyclic heterocycloalkyl, optionally substituted 5-membered and 5-membered fused heterocycloalkyl, optionally substituted 5-membered monocyclic heteroaryl, and optionally substituted 6-membered monocyclic heteroaryl; the “optionally substituted” refers to being unsubstituted or being substituted with one or more R¹⁷; wherein, R¹⁸, at each occurrence, is independently selected from hydrogen, F, Cl, Br, hydroxyl, amino, methyl, ethyl, methylamino, ethylamino, n-propylamino, isopropylamino, dimethylamino, diethylamino, cyclopropylamino, cyclobutylamino, cyclopentylamino, cyclobutylaminomethyl, and cyclobutylaminoethyl;
further preferably, L² is selected from optionally substituted methyl, optionally substituted ethyl, optionally substituted propyl, optionally substituted pyrrolidinyl, optionally substituted azetidinyl, optionally substituted pyrazolyl, optionally substituted piperazinyl, optionally substituted piperidyl, and optionally substituted 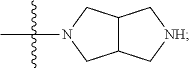 [SMILES: *N1CC2CNCC2C1]
the “optionally substituted” refers to being unsubstituted or being substituted with one or more R¹⁷; wherein, R¹⁷, at each occurrence, is independently selected from hydrogen, F, Cl, Br, hydroxyl, amino, methyl, ethyl, methylamino, ethylamino, n-propylamino, isopropylamino, dimethylamino, cyclopropylamino, cyclobutylamino, cyclopentylamino, cyclobutylaminomethyl, and cyclobutylaminoethyl;
still further preferably, L² is selected from

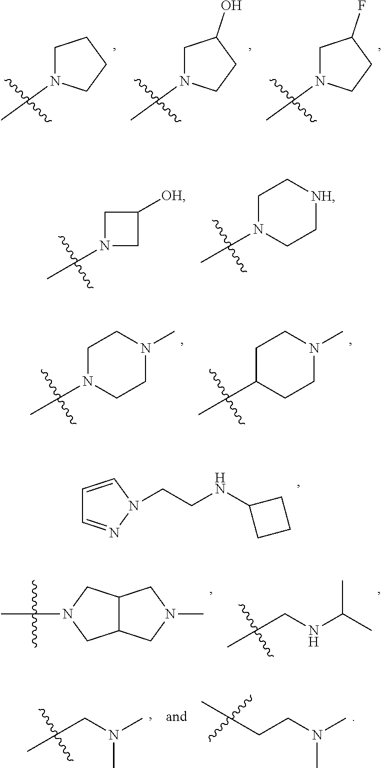

35-51. (canceled)

52. The following compounds, and stereoisomers, optical isomers, pharmaceutically acceptable salts, prodrugs, and solvates thereof:

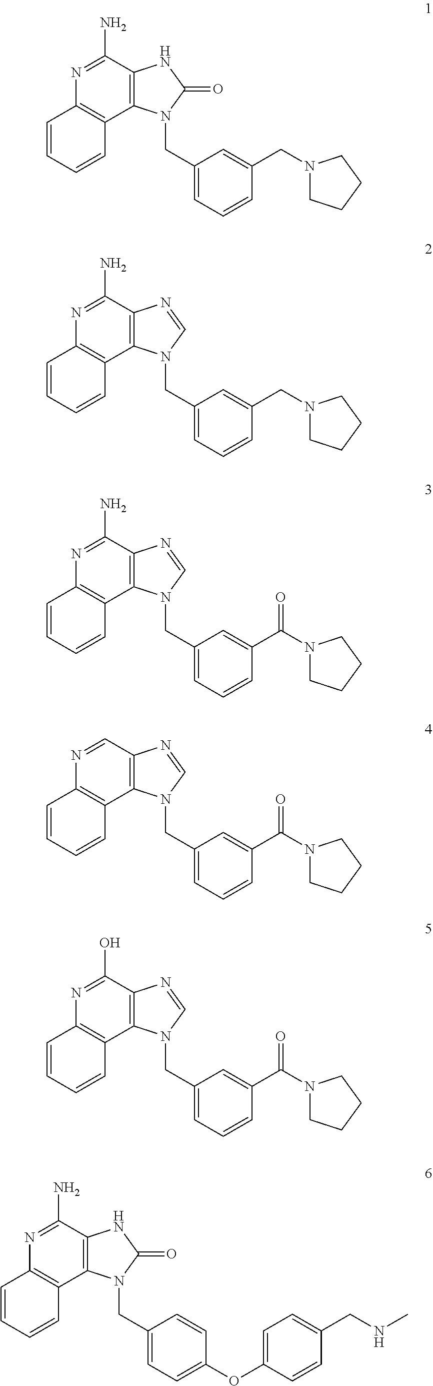 [SMILES: Nc1nc2ccccc2c2c1[nH]c(=O)n2Cc1cccc(CN2CCCC2)c1 ; Nc1nc2ccccc2c2c1ncn2Cc1cccc(CN2CCCC2)c1 ; Nc1nc2ccccc2c2c1ncn2Cc1cccc(C(=O)N2CCCC2)c1 ; O=C(c1cccc(Cn2cnc3cnc4ccccc4c32)c1)N1CCCC1 ; O=C(c1cccc(Cn2cnc3c(O)nc4ccccc4c32)c1)N1CCCC1 ; CNCc1ccc(Oc2ccc(Cn3c(=O)[nH]c4c(N)nc5ccccc5c43)cc2)cc1]

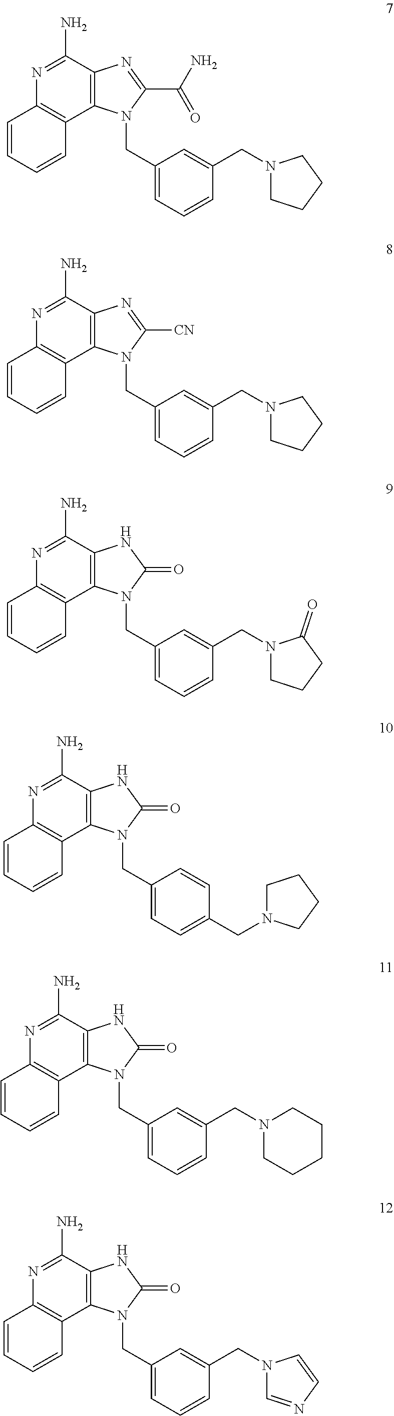 [SMILES: NC(=O)c1nc2c(N)nc3ccccc3c2n1Cc1cccc(CN2CCCC2)c1 ; N#Cc1nc2c(N)nc3ccccc3c2n1Cc1cccc(CN2CCCC2)c1 ; Nc1nc2ccccc2c2c1[nH]c(=O)n2Cc1cccc(CN2CCCC2=O)c1 ; Nc1nc2ccccc2c2c1[nH]c(=O)n2Cc1ccc(CN2CCCC2)cc1 ; Nc1nc2ccccc2c2c1[nH]c(=O)n2Cc1cccc(CN2CCCCC2)c1 ; Nc1nc2ccccc2c2c1[nH]c(=O)n2Cc1cccc(Cn2ccnc2)c1]

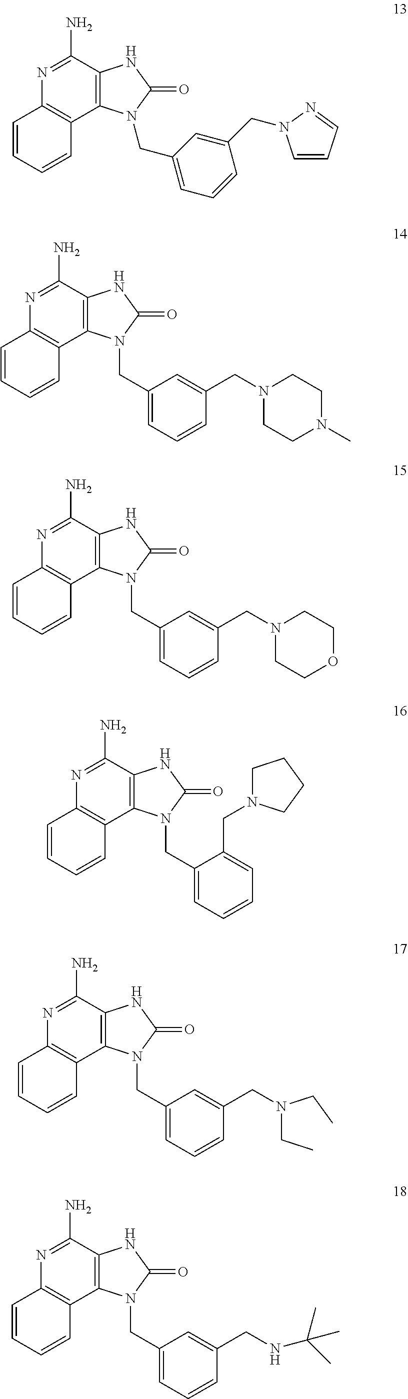 [SMILES: Nc1nc2ccccc2c2c1[nH]c(=O)n2Cc1cccc(Cn2cccn2)c1 ; CN1CCN(Cc2cccc(Cn3c(=O)[nH]c4c(N)nc5ccccc5c43)c2)CC1 ; Nc1nc2ccccc2c2c1[nH]c(=O)n2Cc1cccc(CN2CCOCC2)c1 ; Nc1nc2ccccc2c2c1[nH]c(=O)n2Cc1ccccc1CN1CCCC1 ; CCN(CC)Cc1cccc(Cn2c(=O)[nH]c3c(N)nc4ccccc4c32)c1 ; CC(C)(C)NCc1cccc(Cn2c(=O)[nH]c3c(N)nc4ccccc4c32)c1]

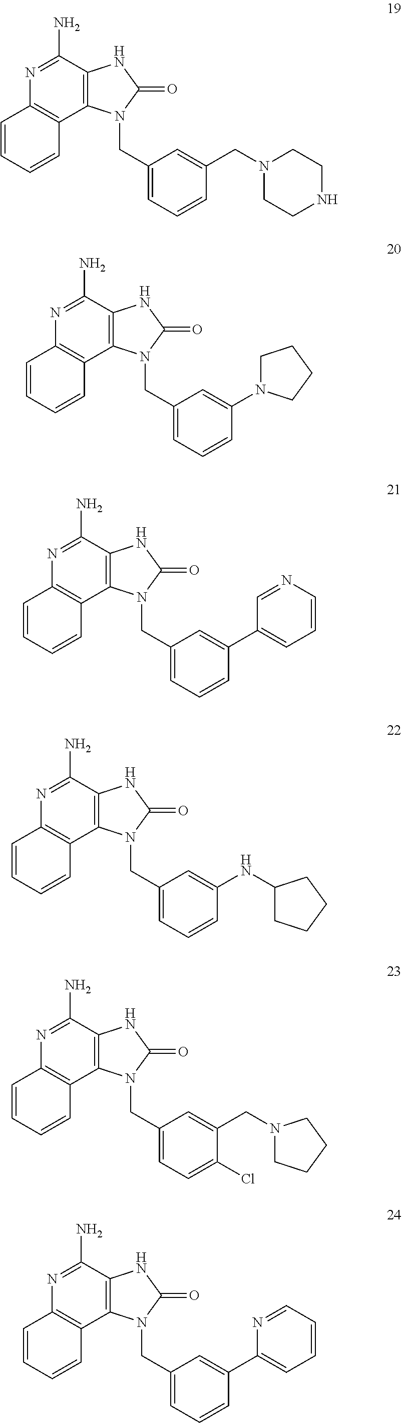 [SMILES: Nc1nc2ccccc2c2c1[nH]c(=O)n2Cc1cccc(CN2CCNCC2)c1 ; Nc1nc2ccccc2c2c1[nH]c(=O)n2Cc1cccc(N2CCCC2)c1 ; Nc1nc2ccccc2c2c1[nH]c(=O)n2Cc1cccc(-c2cccnc2)c1 ; Nc1nc2ccccc2c2c1[nH]c(=O)n2Cc1cccc(NC2CCCC2)c1 ; Nc1nc2ccccc2c2c1[nH]c(=O)n2Cc1ccc(Cl)c(CN2CCCC2)c1 ; Nc1nc2ccccc2c2c1[nH]c(=O)n2Cc1cccc(-c2ccccn2)c1]

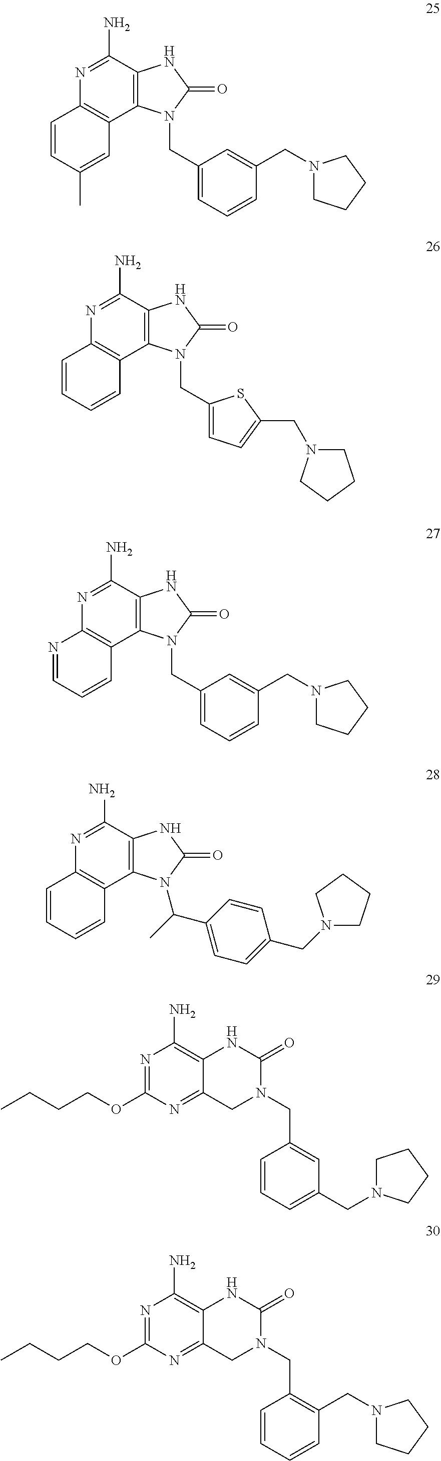 [SMILES: Cc1ccc2nc(N)c3[nH]c(=O)n(Cc4cccc(CN5CCCC5)c4)c3c2c1 ; Nc1nc2ccccc2c2c1[nH]c(=O)n2Cc1ccc(CN2CCCC2)s1 ; Nc1nc2ncccc2c2c1[nH]c(=O)n2Cc1cccc(CN2CCCC2)c1 ; CC(c1ccc(CN2CCCC2)cc1)n1c(=O)[nH]c2c(N)nc3ccccc3c21 ; CCCCOc1nc(N)c2c(n1)CN(Cc1cccc(CN3CCCC3)c1)C(=O)N2 ; CCCCOc1nc(N)c2c(n1)CN(Cc1ccccc1CN1CCCC1)C(=O)N2]

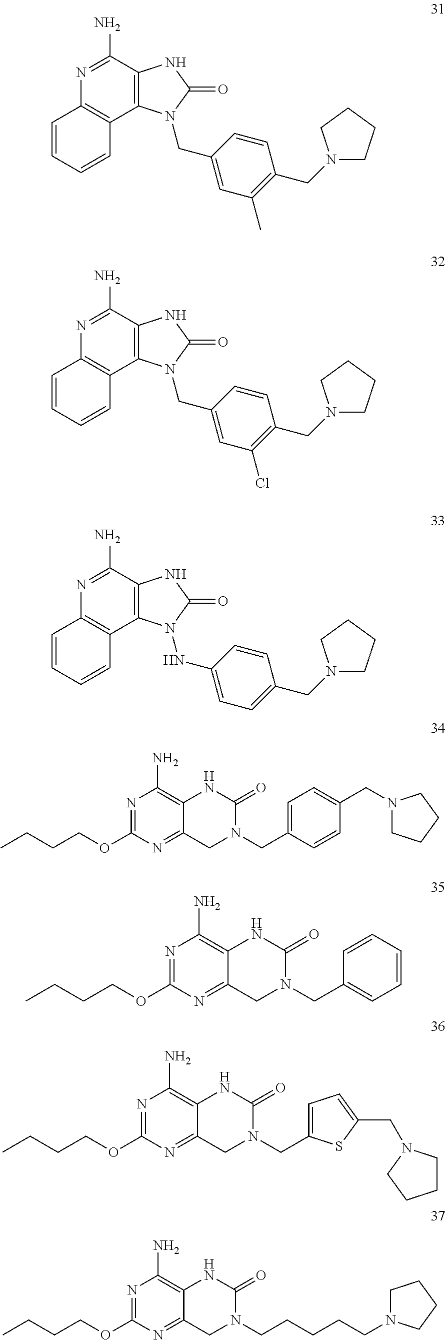

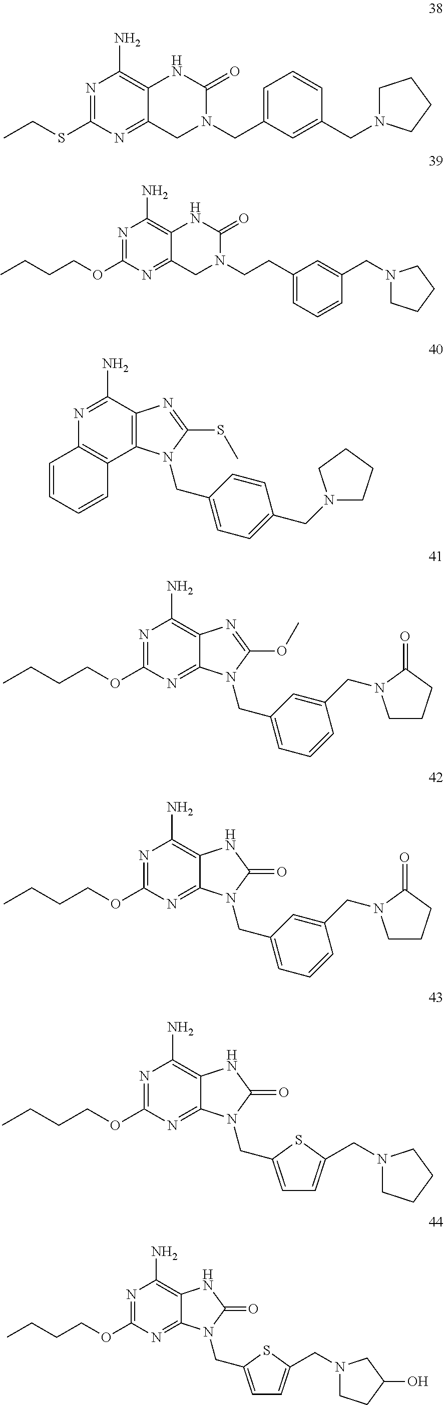

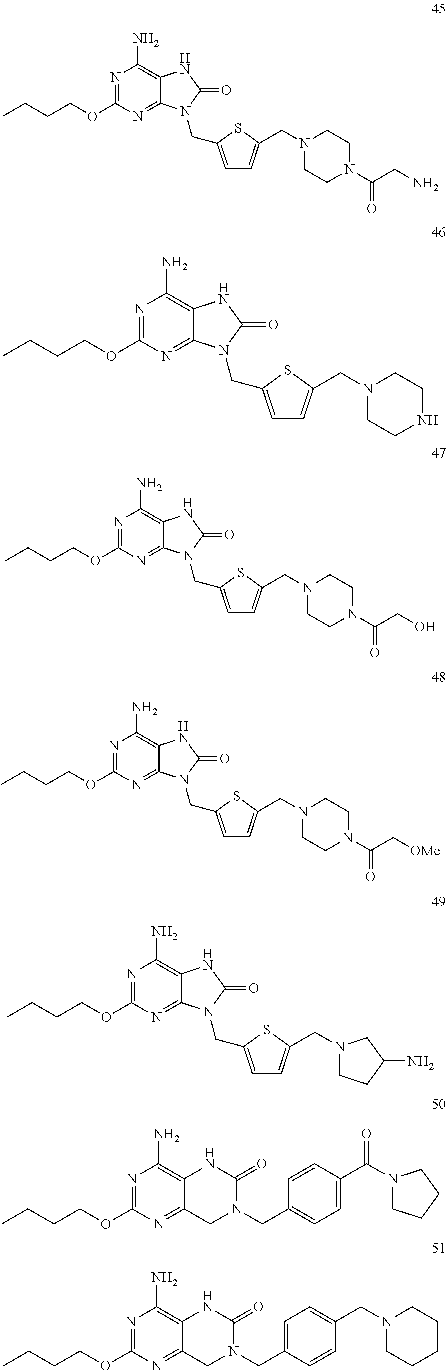

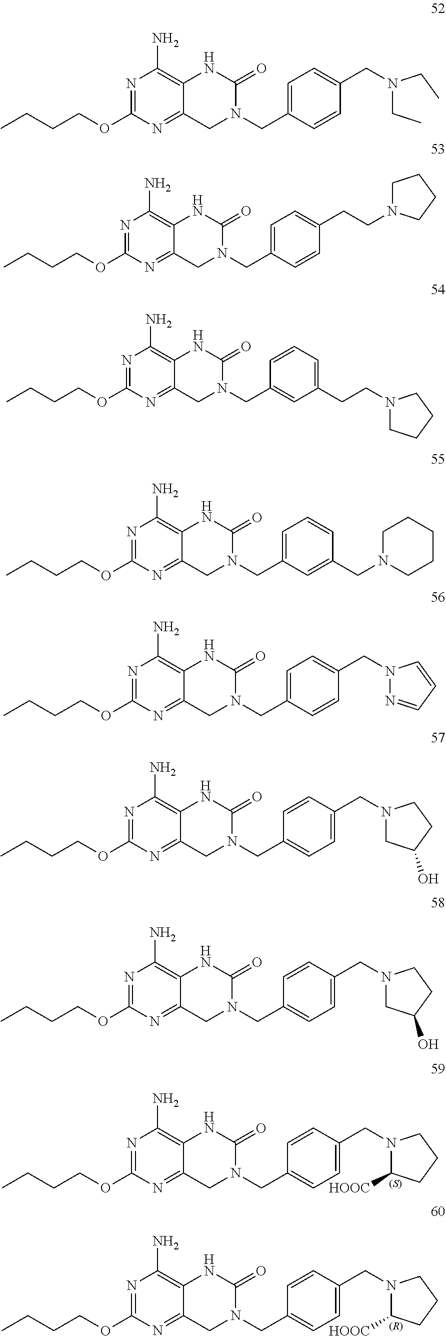

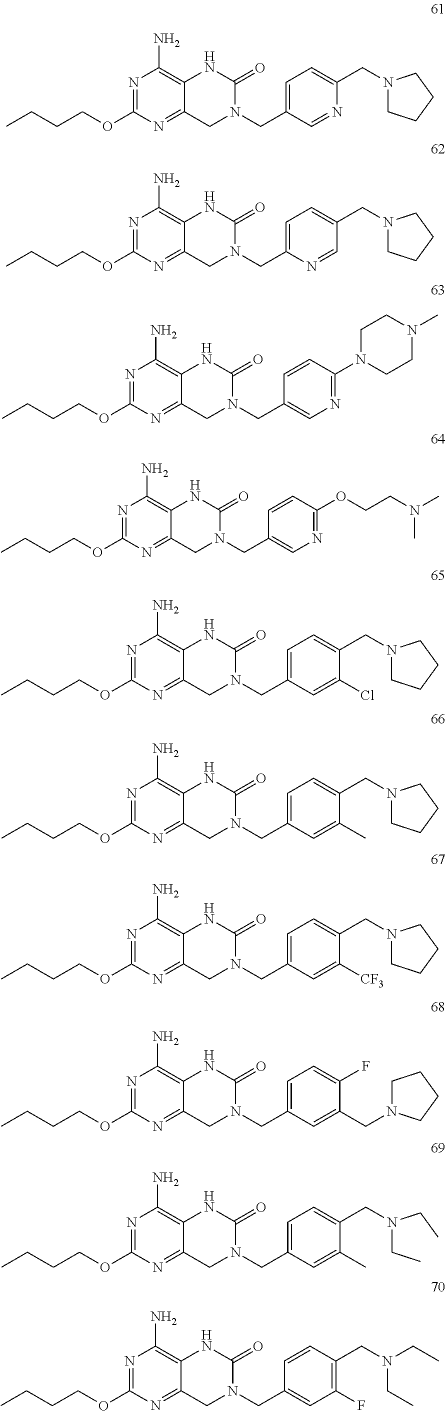

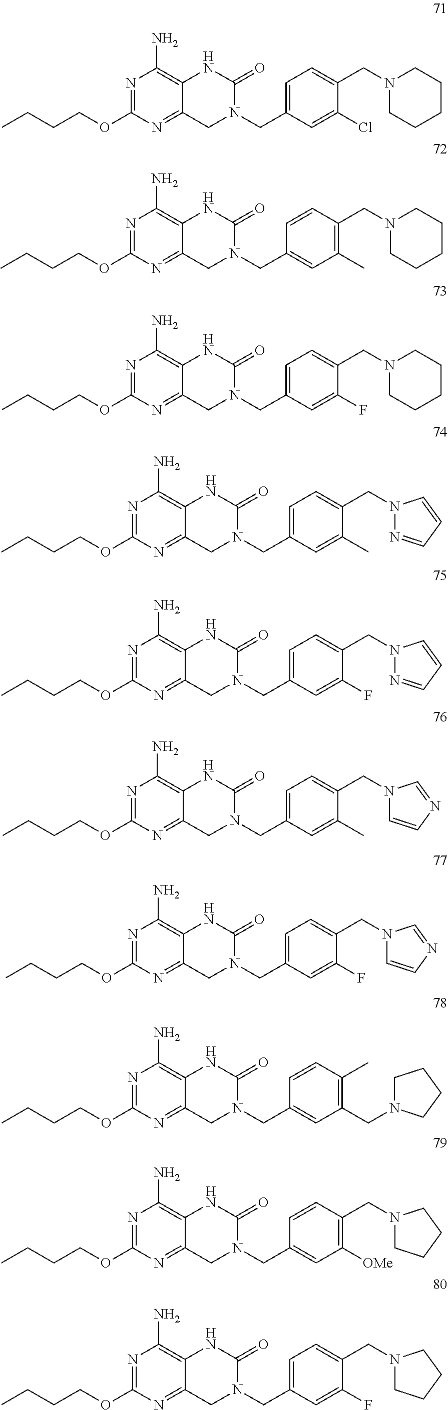

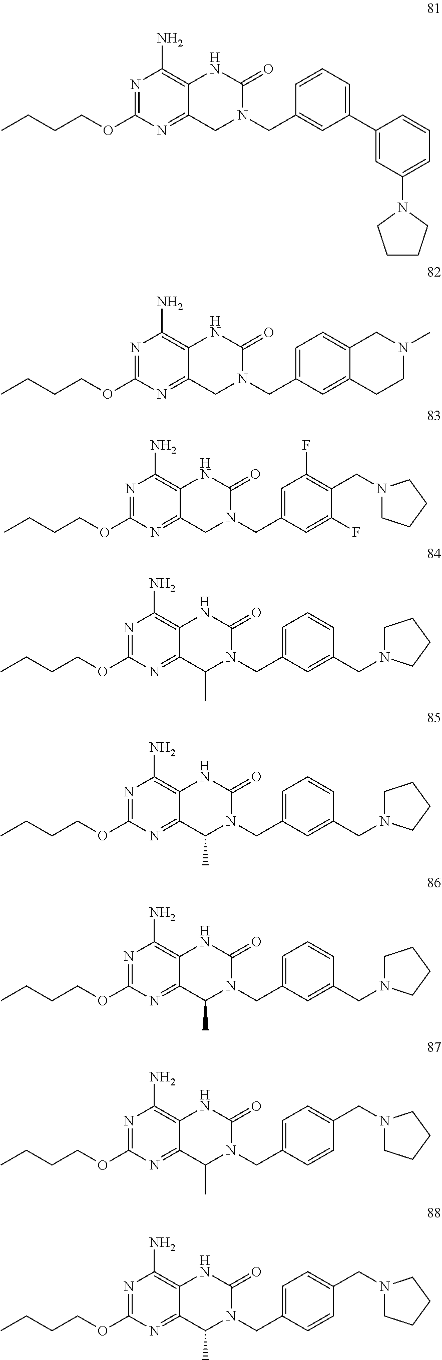

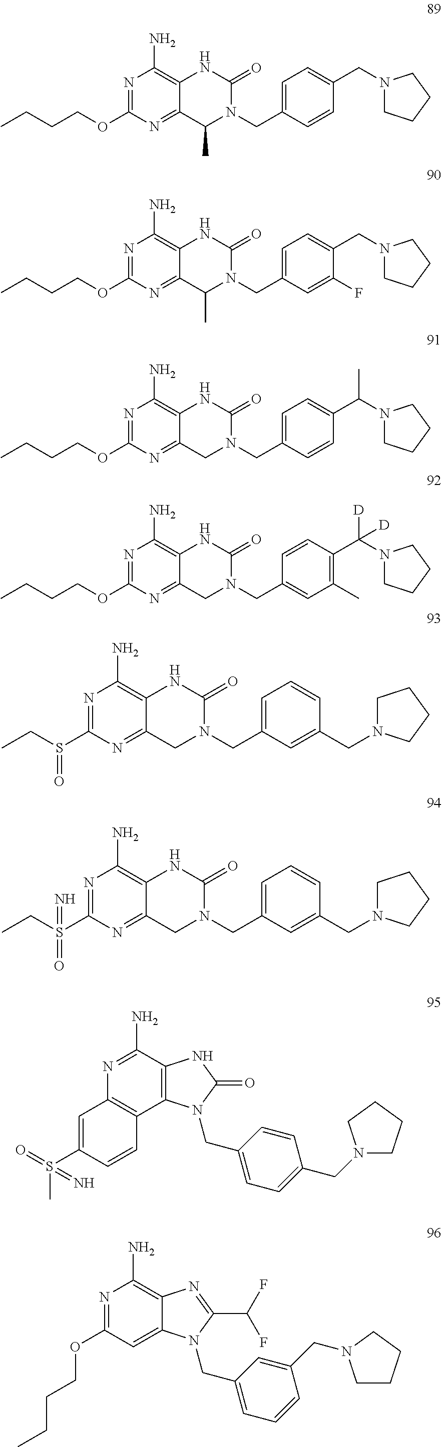

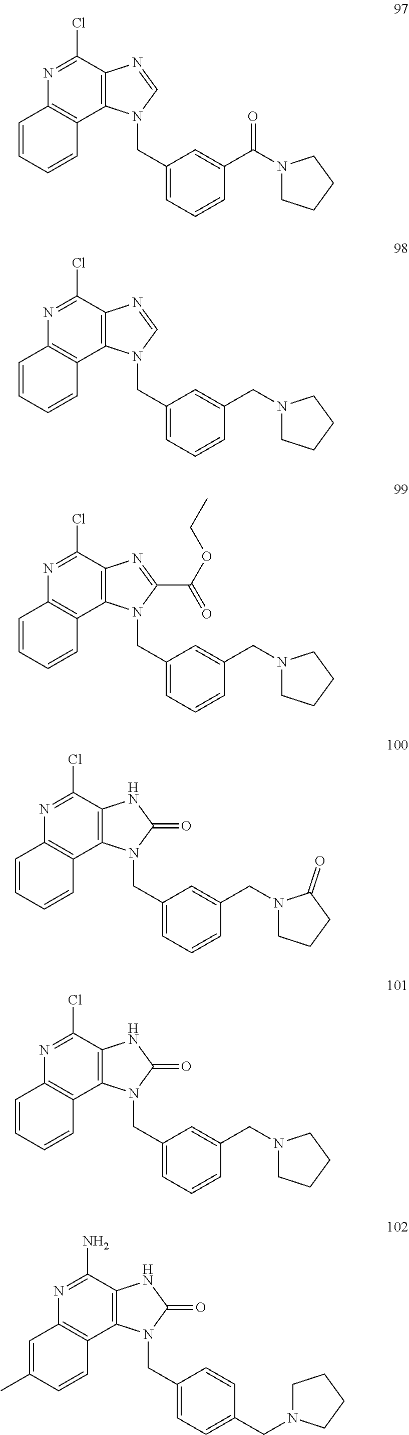

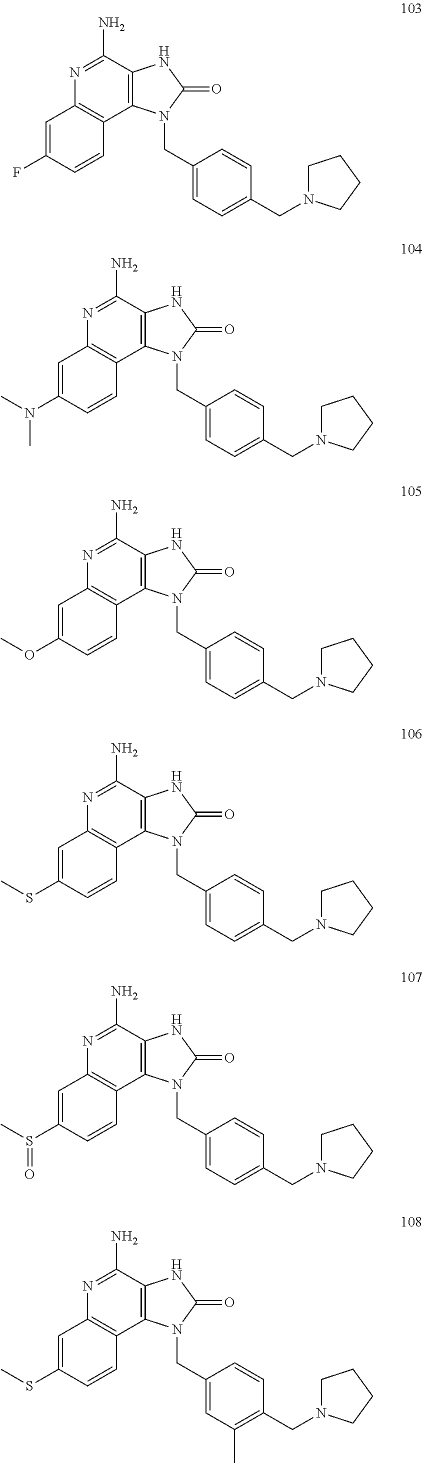 [SMILES: Nc1nc2cc(F)ccc2c2c1[nH]c(=O)n2Cc1ccc(CN2CCCC2)cc1 ; CN(C)c1ccc2c(c1)nc(N)c1[nH]c(=O)n(Cc3ccc(CN4CCCC4)cc3)c12 ; COc1ccc2c(c1)nc(N)c1[nH]c(=O)n(Cc3ccc(CN4CCCC4)cc3)c12 ; CSc1ccc2c(c1)nc(N)c1[nH]c(=O)n(Cc3ccc(CN4CCCC4)cc3)c12 ; CS(=O)c1ccc2c(c1)nc(N)c1[nH]c(=O)n(Cc3ccc(CN4CCCC4)cc3)c12 ; CSc1ccc2c(c1)nc(N)c1[nH]c(=O)n(Cc3ccc(CN4CCCC4)c(C)c3)c12]

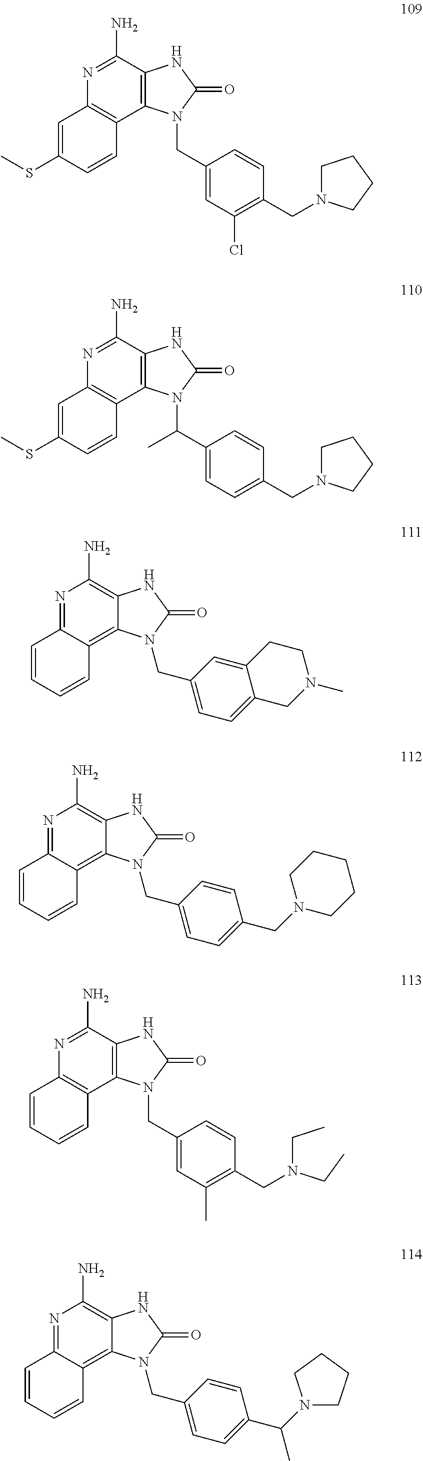 [SMILES: CSc1ccc2c(c1)nc(N)c1[nH]c(=O)n(Cc3ccc(CN4CCCC4)c(Cl)c3)c12 ; CSc1ccc2c(c1)nc(N)c1[nH]c(=O)n(C(C)c3ccc(CN4CCCC4)cc3)c12 ; CN1CCc2cc(Cn3c(=O)[nH]c4c(N)nc5ccccc5c43)ccc2C1 ; Nc1nc2ccccc2c2c1[nH]c(=O)n2Cc1ccc(CN2CCCCC2)cc1 ; CCN(CC)Cc1ccc(Cn2c(=O)[nH]c3c(N)nc4ccccc4c32)cc1C ; CC(c1ccc(Cn2c(=O)[nH]c3c(N)nc4ccccc4c32)cc1)N1CCCC1]

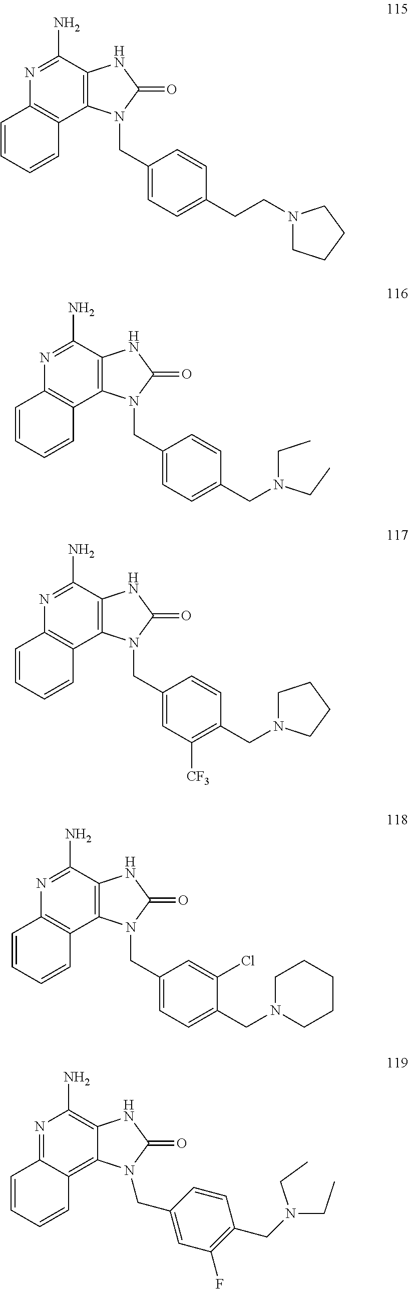 [SMILES: Nc1nc2ccccc2c2c1[nH]c(=O)n2Cc1ccc(CCN2CCCC2)cc1 ; CCN(CC)Cc1ccc(Cn2c(=O)[nH]c3c(N)nc4ccccc4c32)cc1 ; Nc1nc2ccccc2c2c1[nH]c(=O)n2Cc1ccc(CN2CCCC2)c(C(F)(F)F)c1 ; Nc1nc2ccccc2c2c1[nH]c(=O)n2Cc1ccc(CN2CCCCC2)c(Cl)c1 ; CCN(CC)Cc1ccc(Cn2c(=O)[nH]c3c(N)nc4ccccc4c32)cc1F]

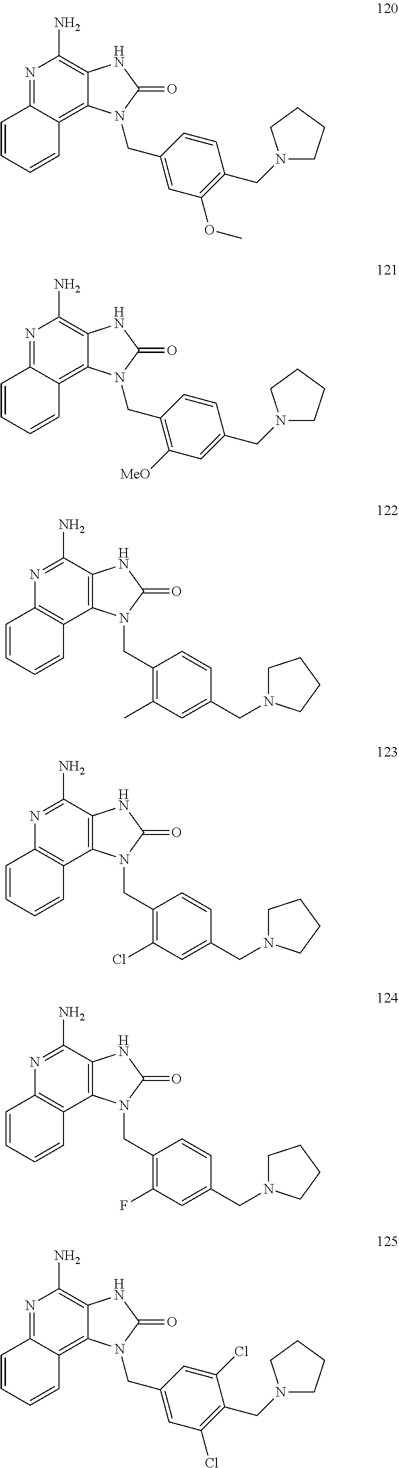 [SMILES: COc1cc(Cn2c(=O)[nH]c3c(N)nc4ccccc4c32)ccc1CN1CCCC1 ; COc1cc(CN2CCCC2)ccc1Cn1c(=O)[nH]c2c(N)nc3ccccc3c21 ; Cc1cc(CN2CCCC2)ccc1Cn1c(=O)[nH]c2c(N)nc3ccccc3c21 ; Nc1nc2ccccc2c2c1[nH]c(=O)n2Cc1ccc(CN2CCCC2)cc1Cl ; Nc1nc2ccccc2c2c1[nH]c(=O)n2Cc1ccc(CN2CCCC2)cc1F ; Nc1nc2ccccc2c2c1[nH]c(=O)n2Cc1cc(Cl)c(CN2CCCC2)c(Cl)c1]

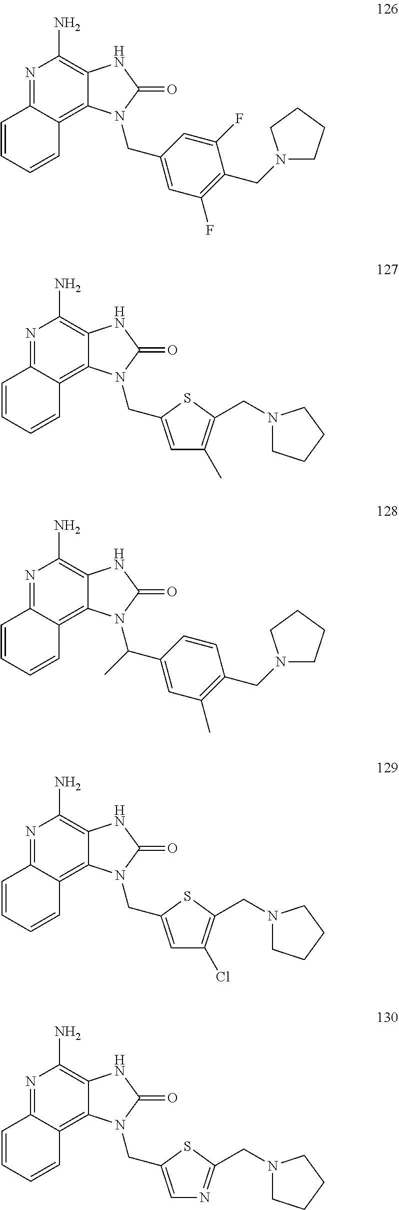 [SMILES: Nc1nc2ccccc2c2c1[nH]c(=O)n2Cc1cc(F)c(CN2CCCC2)c(F)c1 ; Cc1cc(Cn2c(=O)[nH]c3c(N)nc4ccccc4c32)sc1CN1CCCC1 ; Cc1cc(C(C)n2c(=O)[nH]c3c(N)nc4ccccc4c32)ccc1CN1CCCC1 ; Nc1nc2ccccc2c2c1[nH]c(=O)n2Cc1cc(Cl)c(CN2CCCC2)s1 ; Nc1nc2ccccc2c2c1[nH]c(=O)n2Cc1cnc(CN2CCCC2)s1]

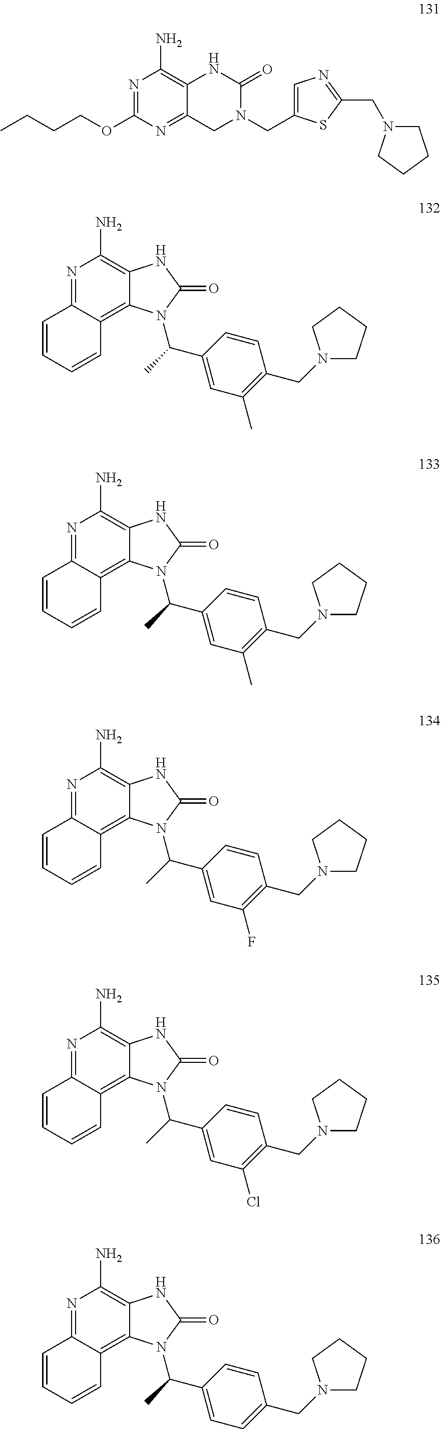 [SMILES: CCCCOc1nc(N)c2c(n1)CN(Cc1cnc(CN3CCCC3)s1)C(=O)N2 ; Cc1cc([C@H](C)n2c(=O)[nH]c3c(N)nc4ccccc4c32)ccc1CN1CCCC1 ; Cc1cc([C@@H](C)n2c(=O)[nH]c3c(N)nc4ccccc4c32)ccc1CN1CCCC1 ; CC(c1ccc(CN2CCCC2)c(F)c1)n1c(=O)[nH]c2c(N)nc3ccccc3c21 ; CC(c1ccc(CN2CCCC2)c(Cl)c1)n1c(=O)[nH]c2c(N)nc3ccccc3c21 ; C[C@H](c1ccc(CN2CCCC2)cc1)n1c(=O)[nH]c2c(N)nc3ccccc3c21]

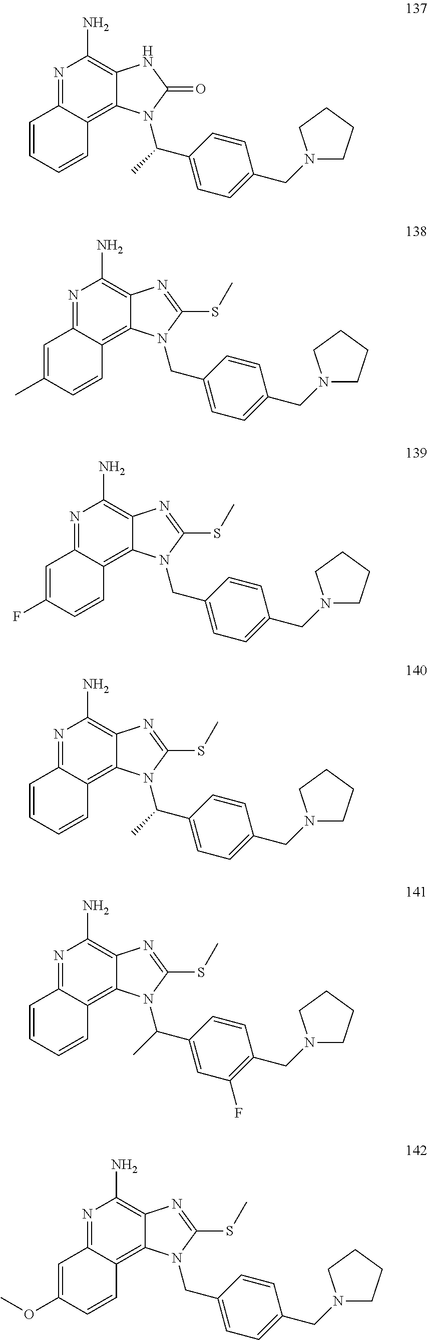 [SMILES: C[C@@H](c1ccc(CN2CCCC2)cc1)n1c(=O)[nH]c2c(N)nc3ccccc3c21 ; CSc1nc2c(N)nc3cc(C)ccc3c2n1Cc1ccc(CN2CCCC2)cc1 ; CSc1nc2c(N)nc3cc(F)ccc3c2n1Cc1ccc(CN2CCCC2)cc1 ; CSc1nc2c(N)nc3ccccc3c2n1[C@@H](C)c1ccc(CN2CCCC2)cc1 ; CSc1nc2c(N)nc3ccccc3c2n1C(C)c1ccc(CN2CCCC2)c(F)c1 ; COc1ccc2c(c1)nc(N)c1nc(SC)n(Cc3ccc(CN4CCCC4)cc3)c12]

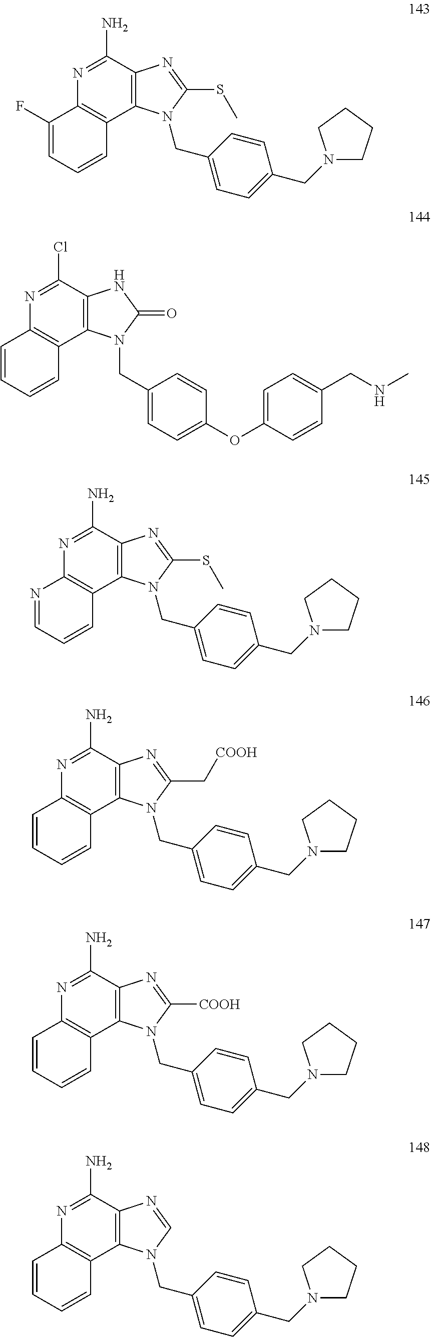 [SMILES: CSc1nc2c(N)nc3c(F)cccc3c2n1Cc1ccc(CN2CCCC2)cc1 ; CNCc1ccc(Oc2ccc(Cn3c(=O)[nH]c4c(Cl)nc5ccccc5c43)cc2)cc1 ; CSc1nc2c(N)nc3ncccc3c2n1Cc1ccc(CN2CCCC2)cc1 ; Nc1nc2ccccc2c2c1nc(CC(=O)O)n2Cc1ccc(CN2CCCC2)cc1 ; Nc1nc2ccccc2c2c1nc(C(=O)O)n2Cc1ccc(CN2CCCC2)cc1 ; Nc1nc2ccccc2c2c1ncn2Cc1ccc(CN2CCCC2)cc1]

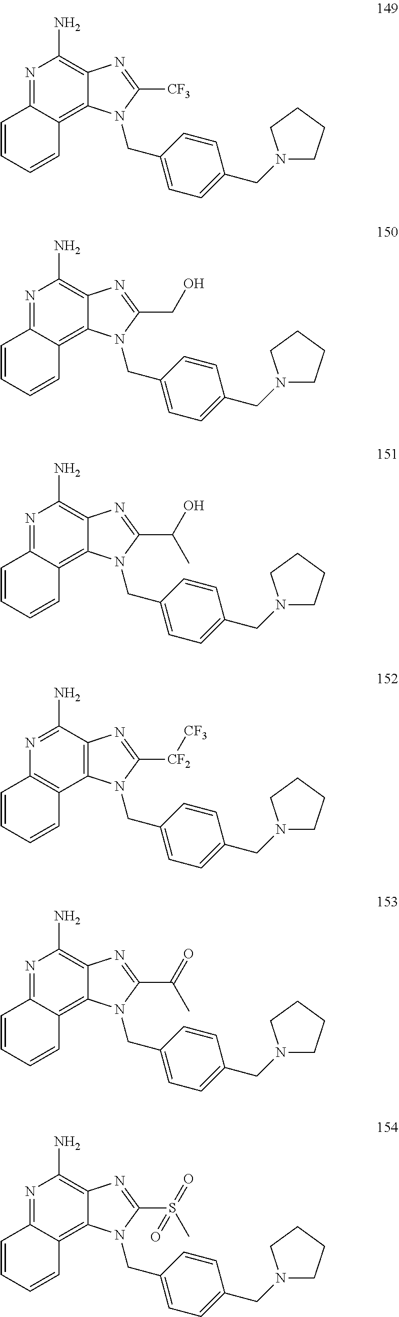 [SMILES: Nc1nc2ccccc2c2c1nc(C(F)(F)F)n2Cc1ccc(CN2CCCC2)cc1 ; Nc1nc2ccccc2c2c1nc(CO)n2Cc1ccc(CN2CCCC2)cc1 ; CC(O)c1nc2c(N)nc3ccccc3c2n1Cc1ccc(CN2CCCC2)cc1 ; Nc1nc2ccccc2c2c1nc(C(F)(F)C(F)(F)F)n2Cc1ccc(CN2CCCC2)cc1 ; CC(=O)c1nc2c(N)nc3ccccc3c2n1Cc1ccc(CN2CCCC2)cc1 ; CS(=O)(=O)c1nc2c(N)nc3ccccc3c2n1Cc1ccc(CN2CCCC2)cc1]

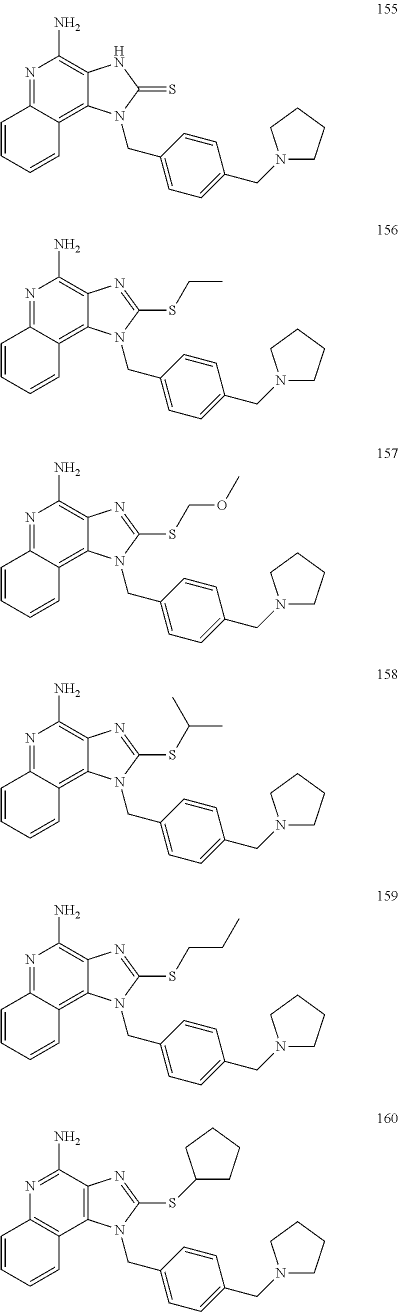

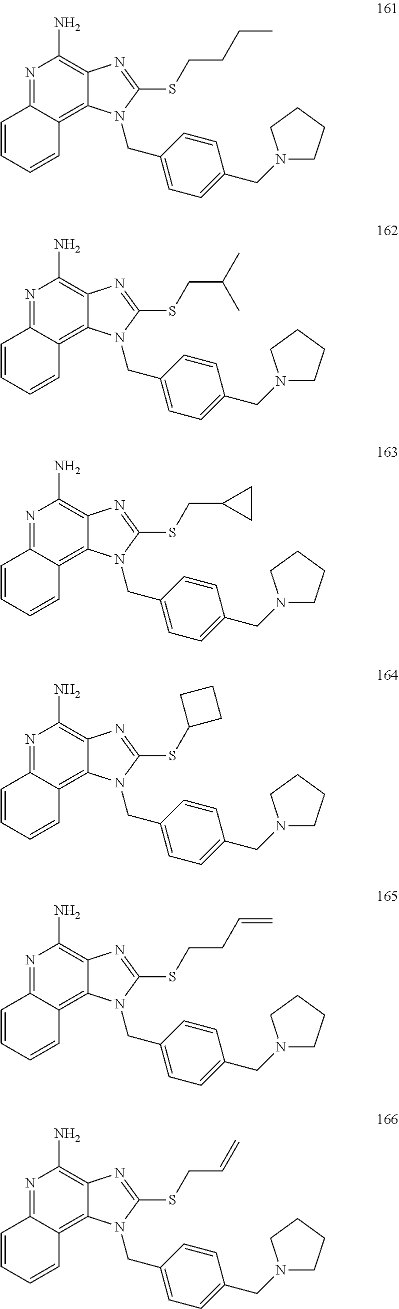

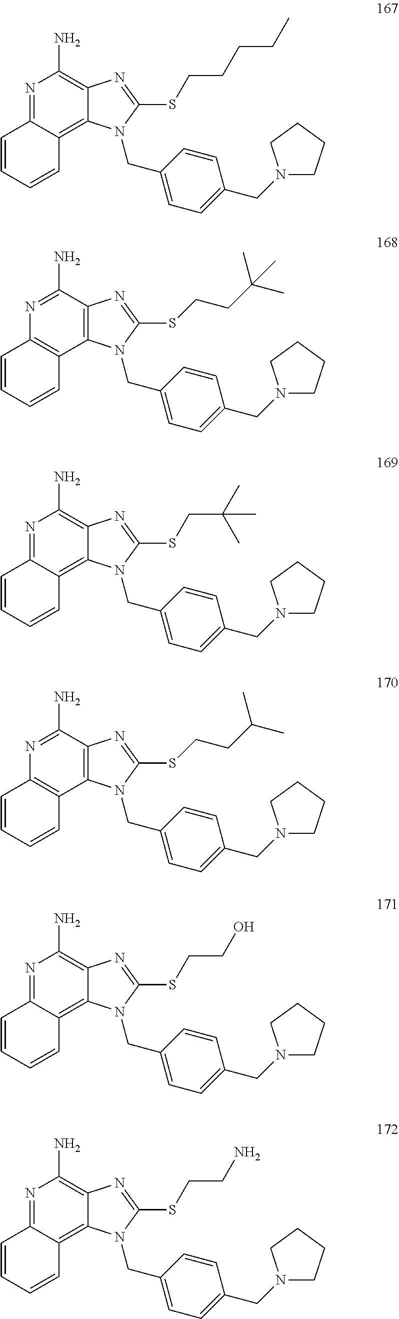 [SMILES: CCCCCSc1nc2c(N)nc3ccccc3c2n1Cc1ccc(CN2CCCC2)cc1 ; CC(C)(C)CCSc1nc2c(N)nc3ccccc3c2n1Cc1ccc(CN2CCCC2)cc1 ; CC(C)(C)CSc1nc2c(N)nc3ccccc3c2n1Cc1ccc(CN2CCCC2)cc1 ; CC(C)CCSc1nc2c(N)nc3ccccc3c2n1Cc1ccc(CN2CCCC2)cc1 ; Nc1nc2ccccc2c2c1nc(SCCO)n2Cc1ccc(CN2CCCC2)cc1 ; NCCSc1nc2c(N)nc3ccccc3c2n1Cc1ccc(CN2CCCC2)cc1]

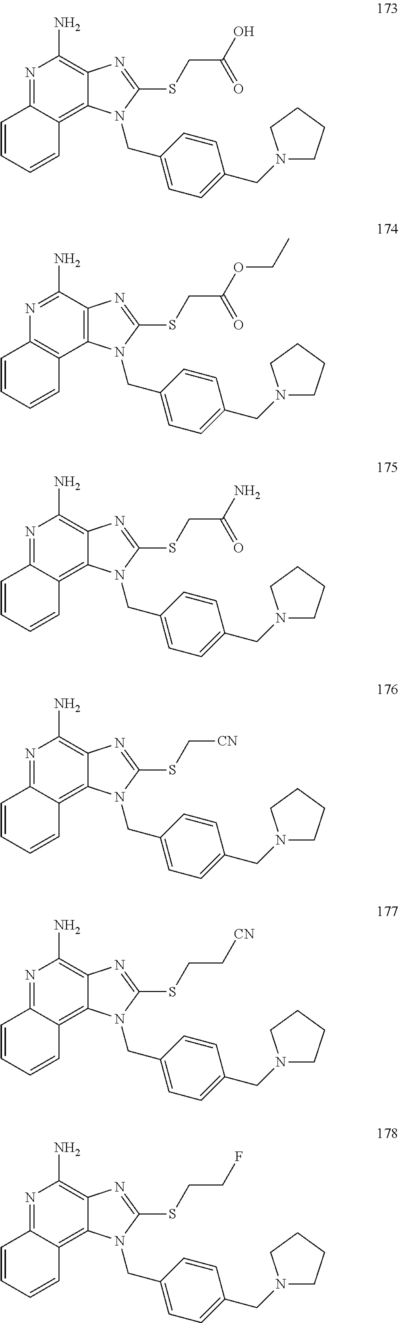 [SMILES: Nc1nc2ccccc2c2c1nc(SCC(=O)O)n2Cc1ccc(CN2CCCC2)cc1 ; CCOC(=O)CSc1nc2c(N)nc3ccccc3c2n1Cc1ccc(CN2CCCC2)cc1 ; NC(=O)CSc1nc2c(N)nc3ccccc3c2n1Cc1ccc(CN2CCCC2)cc1 ; N#CCSc1nc2c(N)nc3ccccc3c2n1Cc1ccc(CN2CCCC2)cc1 ; N#CCCSc1nc2c(N)nc3ccccc3c2n1Cc1ccc(CN2CCCC2)cc1 ; Nc1nc2ccccc2c2c1nc(SCCF)n2Cc1ccc(CN2CCCC2)cc1]

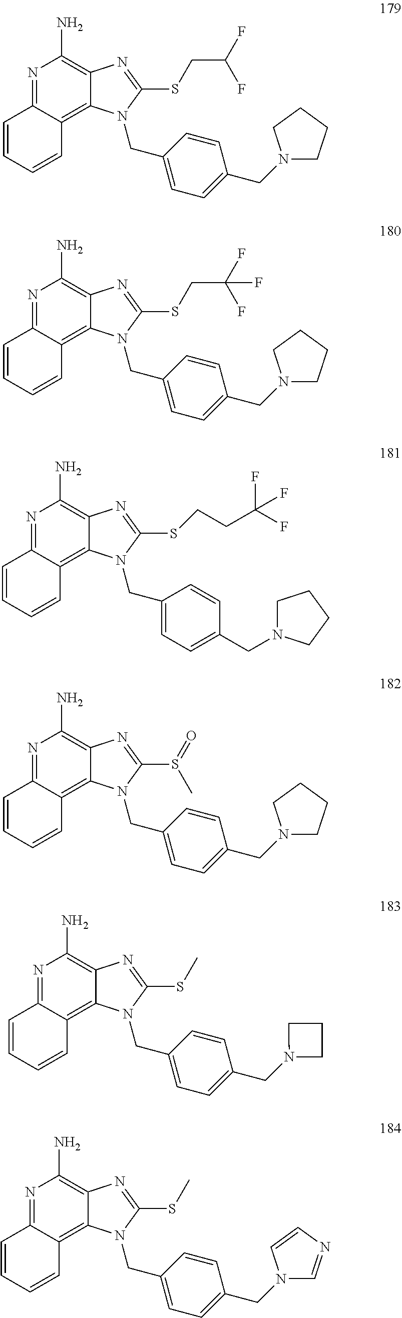 [SMILES: Nc1nc2ccccc2c2c1nc(SCC(F)F)n2Cc1ccc(CN2CCCC2)cc1 ; Nc1nc2ccccc2c2c1nc(SCC(F)(F)F)n2Cc1ccc(CN2CCCC2)cc1 ; Nc1nc2ccccc2c2c1nc(SCCC(F)(F)F)n2Cc1ccc(CN2CCCC2)cc1 ; CS(=O)c1nc2c(N)nc3ccccc3c2n1Cc1ccc(CN2CCCC2)cc1 ; CSc1nc2c(N)nc3ccccc3c2n1Cc1ccc(CN2CCC2)cc1 ; CSc1nc2c(N)nc3ccccc3c2n1Cc1ccc(Cn2ccnc2)cc1]

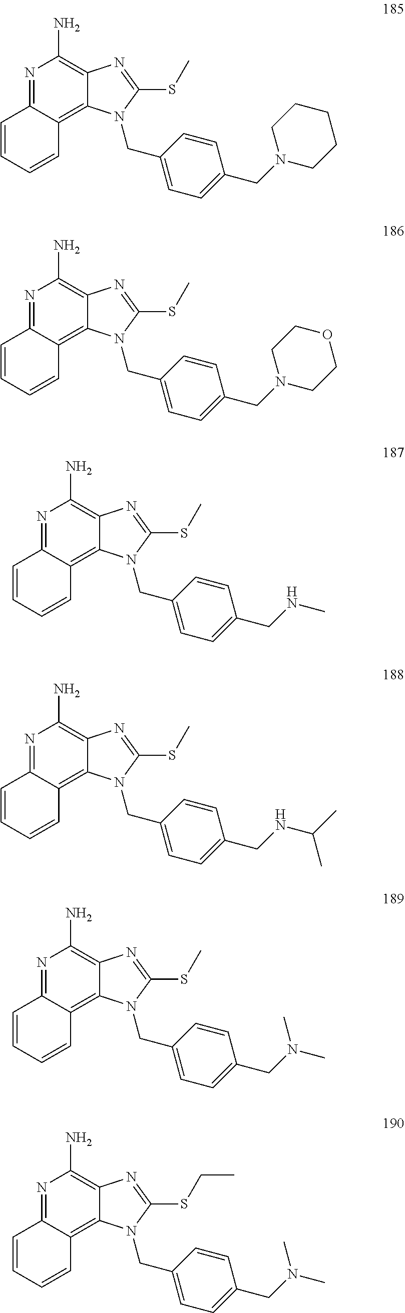 [SMILES: CSc1nc2c(N)nc3ccccc3c2n1Cc1ccc(CN2CCCCC2)cc1 ; CSc1nc2c(N)nc3ccccc3c2n1Cc1ccc(CN2CCOCC2)cc1 ; CNCc1ccc(Cn2c(SC)nc3c(N)nc4ccccc4c32)cc1 ; CSc1nc2c(N)nc3ccccc3c2n1Cc1ccc(CNC(C)C)cc1 ; CSc1nc2c(N)nc3ccccc3c2n1Cc1ccc(CN(C)C)cc1 ; CCSc1nc2c(N)nc3ccccc3c2n1Cc1ccc(CN(C)C)cc1]

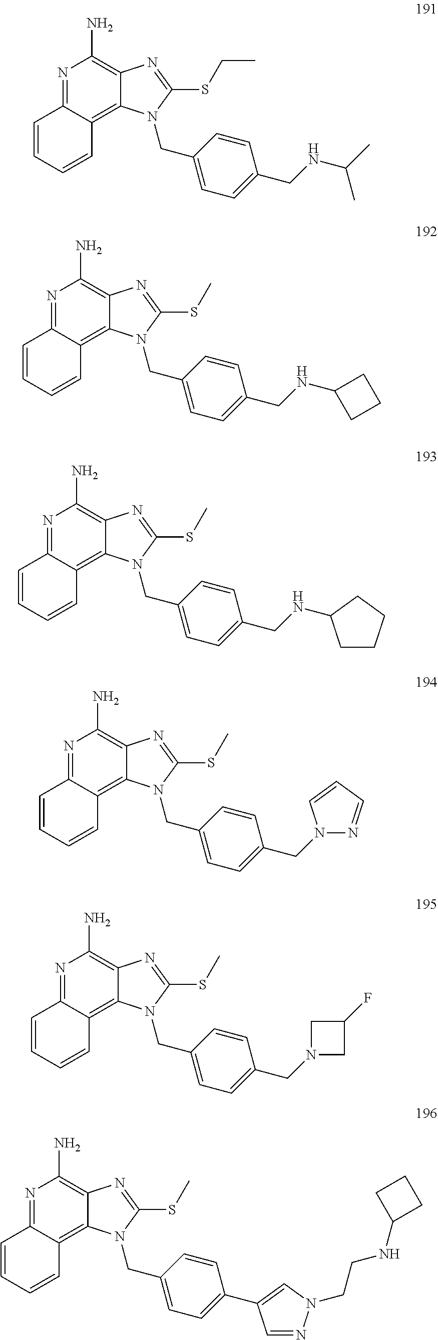 [SMILES: CCSc1nc2c(N)nc3ccccc3c2n1Cc1ccc(CNC(C)C)cc1 ; CSc1nc2c(N)nc3ccccc3c2n1Cc1ccc(CNC2CCC2)cc1 ; CSc1nc2c(N)nc3ccccc3c2n1Cc1ccc(CNC2CCCC2)cc1 ; CSc1nc2c(N)nc3ccccc3c2n1Cc1ccc(Cn2cccn2)cc1 ; CSc1nc2c(N)nc3ccccc3c2n1Cc1ccc(CN2CC(F)C2)cc1 ; CSc1nc2c(N)nc3ccccc3c2n1Cc1ccc(-c2cnn(CCNC3CCC3)c2)cc1]

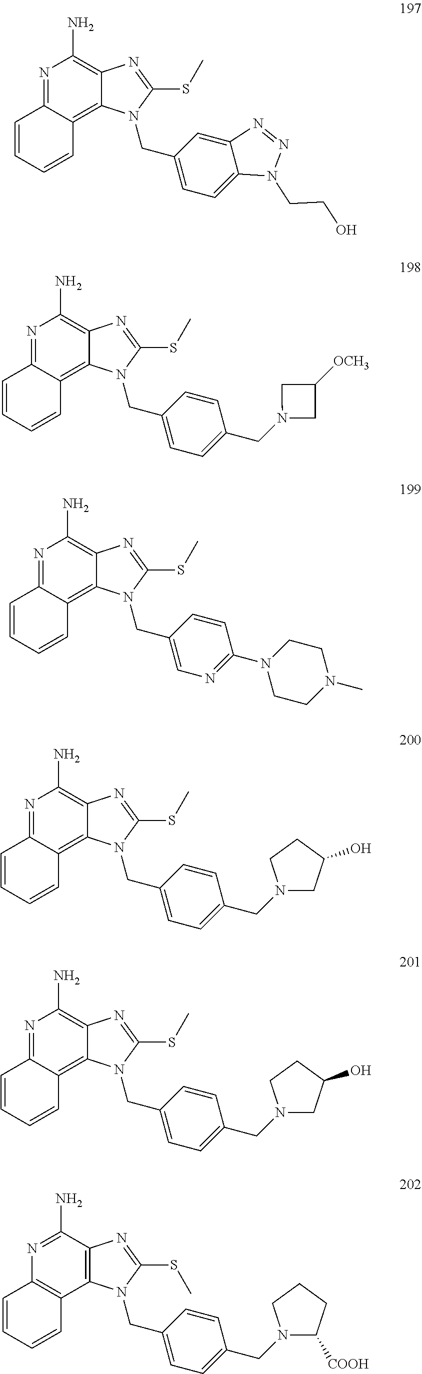 [SMILES: CSc1nc2c(N)nc3ccccc3c2n1Cc1ccc2c(c1)nnn2CCO ; COC1CN(Cc2ccc(Cn3c(SC)nc4c(N)nc5ccccc5c43)cc2)C1 ; CSc1nc2c(N)nc3ccccc3c2n1Cc1ccc(N2CCN(C)CC2)nc1 ; CSc1nc2c(N)nc3ccccc3c2n1Cc1ccc(CN2CC[C@H](O)C2)cc1 ; CSc1nc2c(N)nc3ccccc3c2n1Cc1ccc(CN2CC[C@@H](O)C2)cc1 ; CSc1nc2c(N)nc3ccccc3c2n1Cc1ccc(CN2CCC[C@@H]2C(=O)O)cc1]

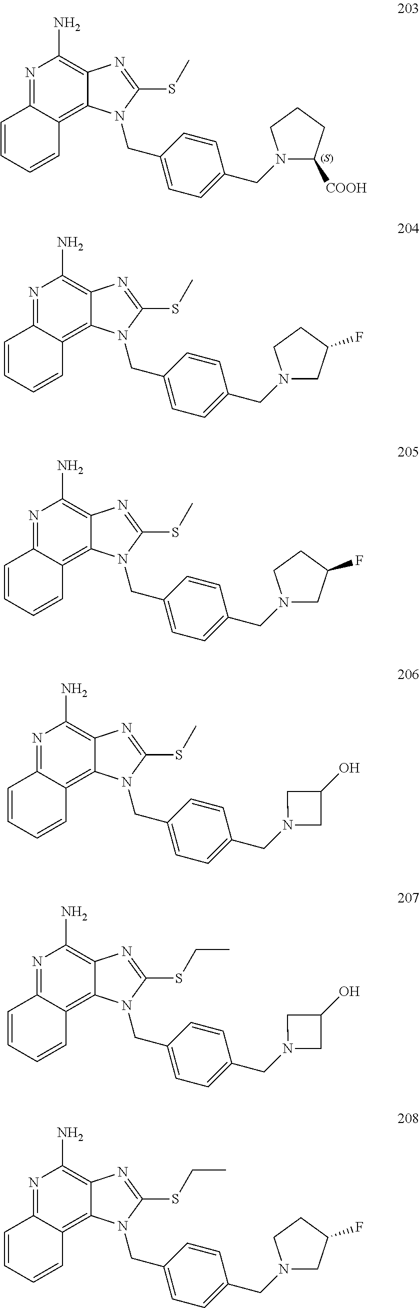

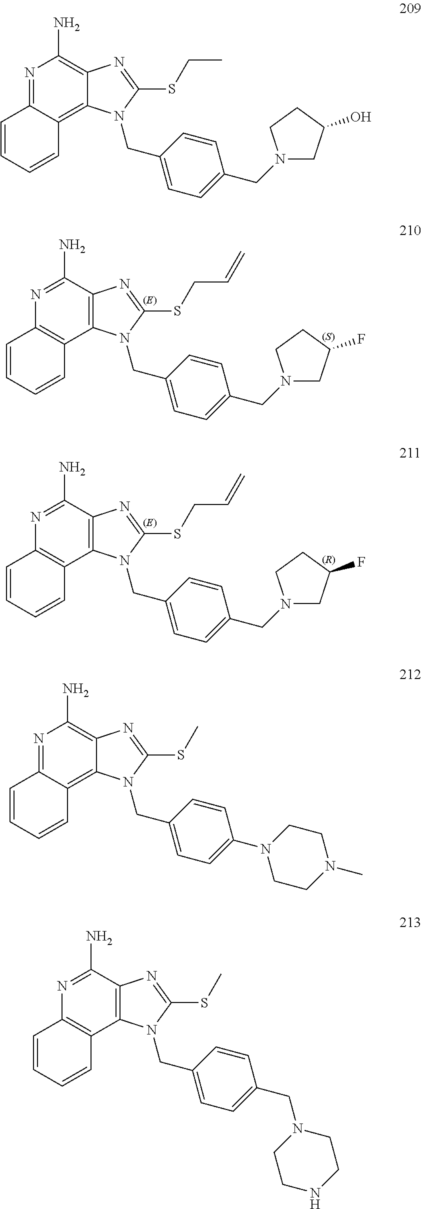

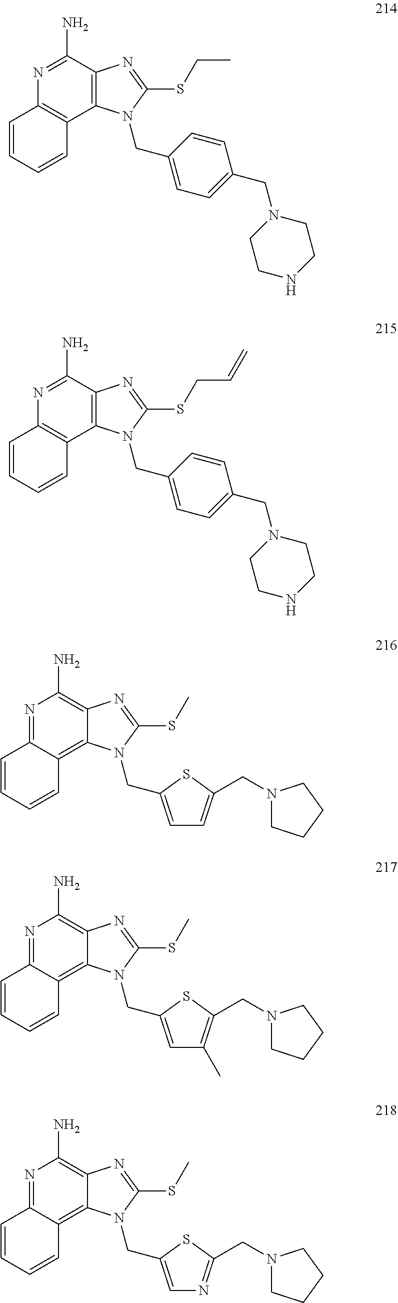 [SMILES: CCSc1nc2c(N)nc3ccccc3c2n1Cc1ccc(CN2CCNCC2)cc1 ; C=CCSc1nc2c(N)nc3ccccc3c2n1Cc1ccc(CN2CCNCC2)cc1 ; CSc1nc2c(N)nc3ccccc3c2n1Cc1ccc(CN2CCCC2)s1 ; CSc1nc2c(N)nc3ccccc3c2n1Cc1cc(C)c(CN2CCCC2)s1 ; CSc1nc2c(N)nc3ccccc3c2n1Cc1cnc(CN2CCCC2)s1]

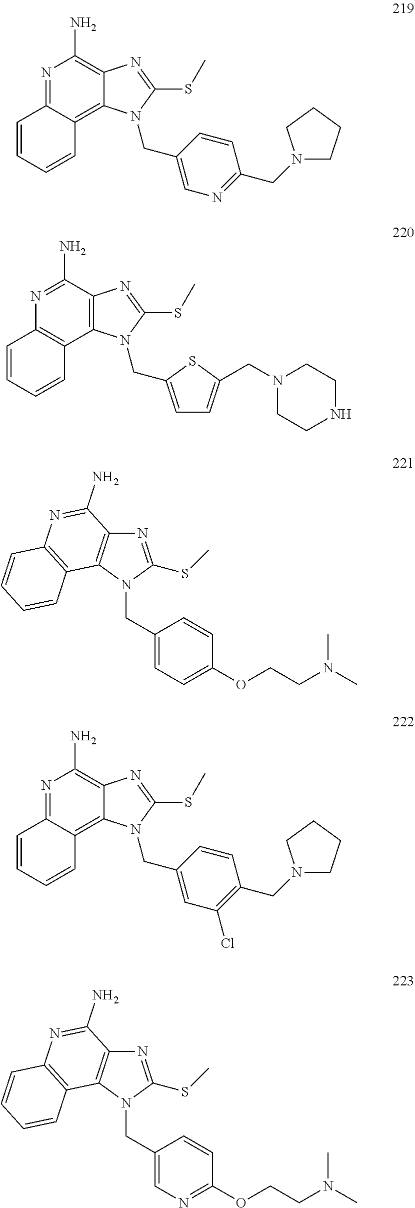 [SMILES: CSc1nc2c(N)nc3ccccc3c2n1Cc1ccc(CN2CCCC2)nc1 ; CSc1nc2c(N)nc3ccccc3c2n1Cc1ccc(CN2CCNCC2)s1 ; CSc1nc2c(N)nc3ccccc3c2n1Cc1ccc(OCCN(C)C)cc1 ; CSc1nc2c(N)nc3ccccc3c2n1Cc1ccc(CN2CCCC2)c(Cl)c1 ; CSc1nc2c(N)nc3ccccc3c2n1Cc1ccc(OCCN(C)C)nc1]

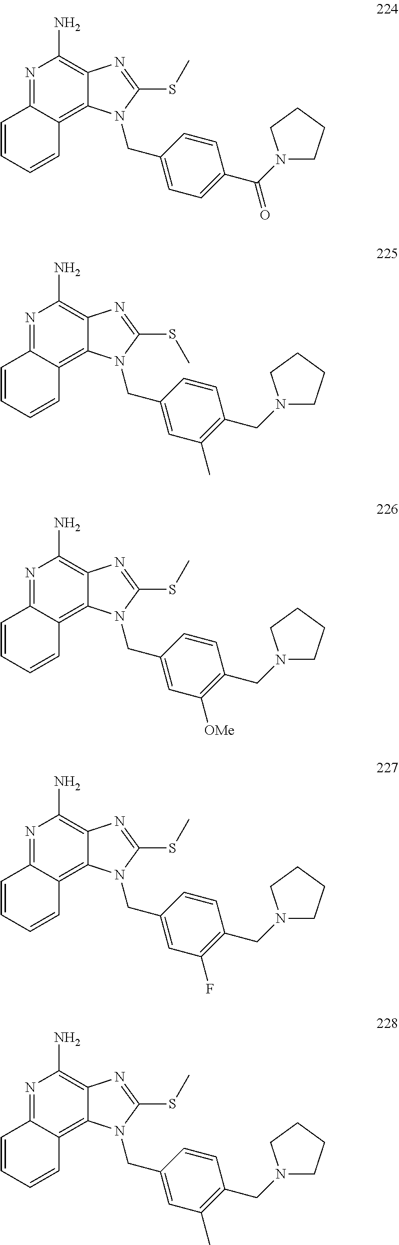 [SMILES: CSc1nc2c(N)nc3ccccc3c2n1Cc1ccc(C(=O)N2CCCC2)cc1 ; CSc1nc2c(N)nc3ccccc3c2n1Cc1ccc(CN2CCCC2)c(C)c1 ; COc1cc(Cn2c(SC)nc3c(N)nc4ccccc4c32)ccc1CN1CCCC1 ; CSc1nc2c(N)nc3ccccc3c2n1Cc1ccc(CN2CCCC2)c(F)c1 ; CSc1nc2c(N)nc3ccccc3c2n1Cc1ccc(CN2CCCC2)c(C)c1]

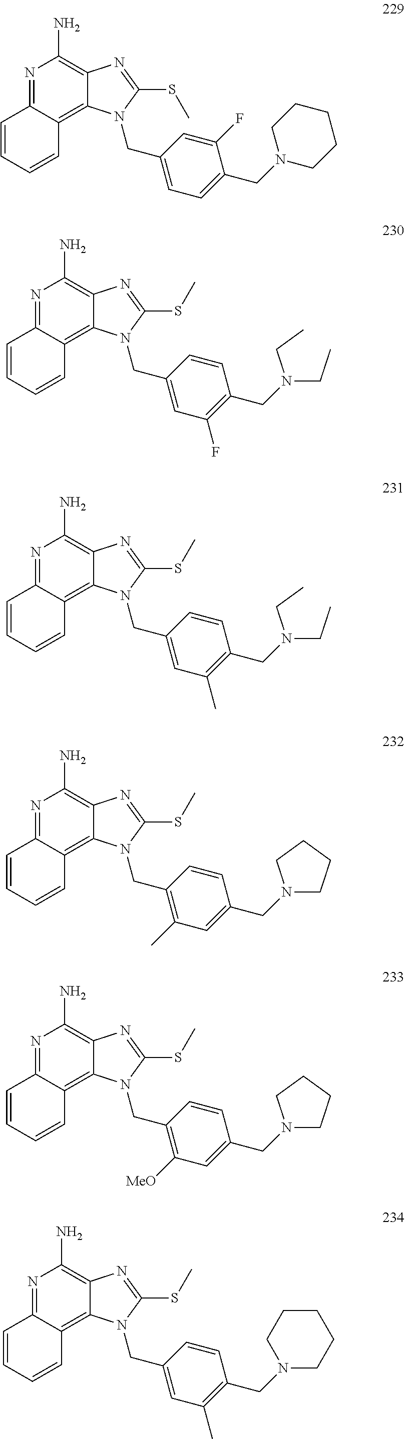 [SMILES: CSc1nc2c(N)nc3ccccc3c2n1Cc1ccc(CN2CCCCC2)c(F)c1 ; CCN(CC)Cc1ccc(Cn2c(SC)nc3c(N)nc4ccccc4c32)cc1F ; CCN(CC)Cc1ccc(Cn2c(SC)nc3c(N)nc4ccccc4c32)cc1C ; CSc1nc2c(N)nc3ccccc3c2n1Cc1ccc(CN2CCCC2)cc1C ; COc1cc(CN2CCCC2)ccc1Cn1c(SC)nc2c(N)nc3ccccc3c21 ; CSc1nc2c(N)nc3ccccc3c2n1Cc1ccc(CN2CCCCC2)c(C)c1]

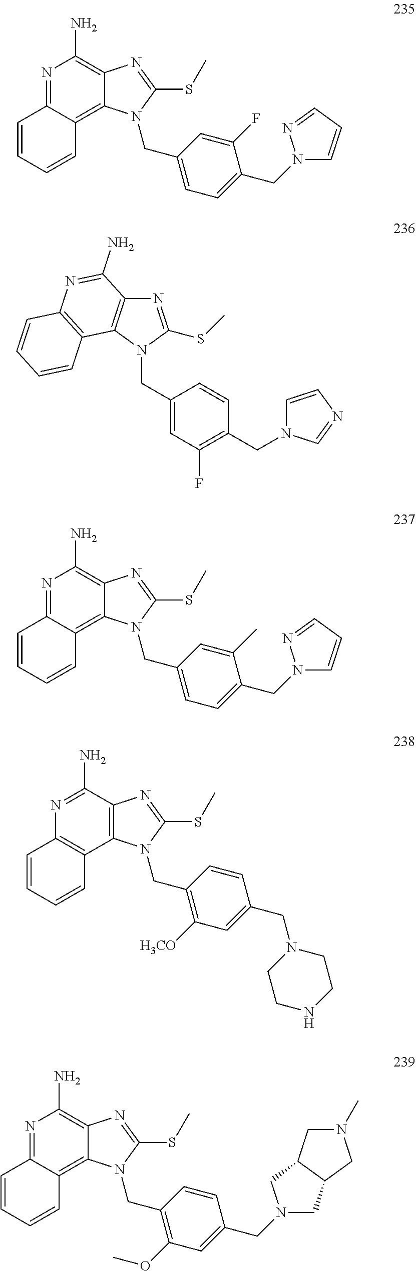 [SMILES: CSc1nc2c(N)nc3ccccc3c2n1Cc1ccc(Cn2cccn2)c(F)c1 ; CSc1nc2c(N)nc3ccccc3c2n1Cc1ccc(Cn2ccnc2)c(F)c1 ; CSc1nc2c(N)nc3ccccc3c2n1Cc1ccc(Cn2cccn2)c(C)c1 ; COc1cc(CN2CCNCC2)ccc1Cn1c(SC)nc2c(N)nc3ccccc3c21 ; COc1cc(CN2C[C@H]3CN(C)C[C@H]3C2)ccc1Cn1c(SC)nc2c(N)nc3ccccc3c21]

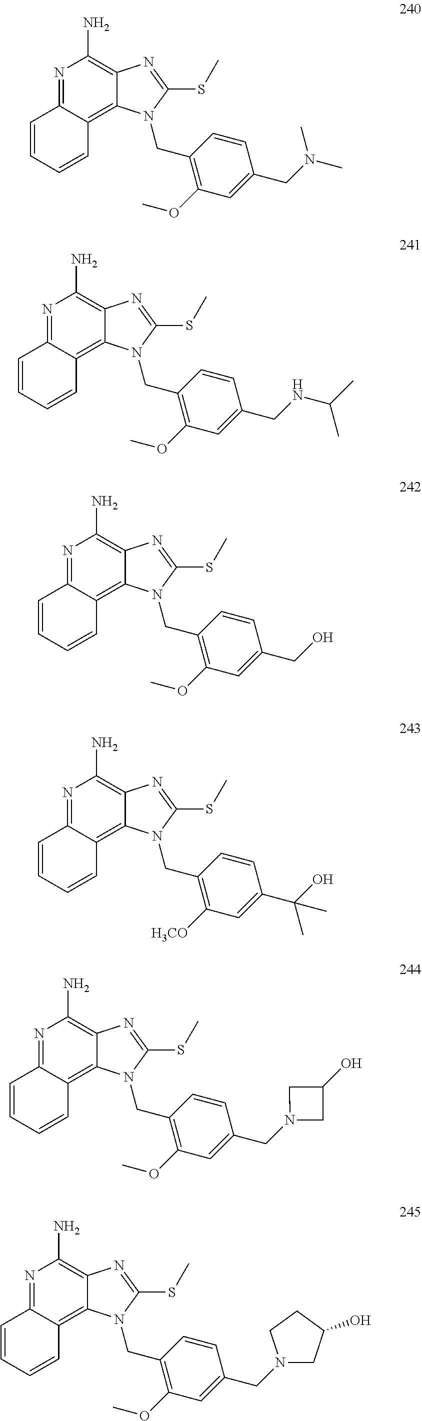 [SMILES: COc1cc(CN(C)C)ccc1Cn1c(SC)nc2c(N)nc3ccccc3c21 ; COc1cc(CNC(C)C)ccc1Cn1c(SC)nc2c(N)nc3ccccc3c21 ; COc1cc(CO)ccc1Cn1c(SC)nc2c(N)nc3ccccc3c21 ; COc1cc(C(C)(C)O)ccc1Cn1c(SC)nc2c(N)nc3ccccc3c21 ; COc1cc(CN2CC(O)C2)ccc1Cn1c(SC)nc2c(N)nc3ccccc3c21 ; COc1cc(CN2CC[C@H](O)C2)ccc1Cn1c(SC)nc2c(N)nc3ccccc3c21]

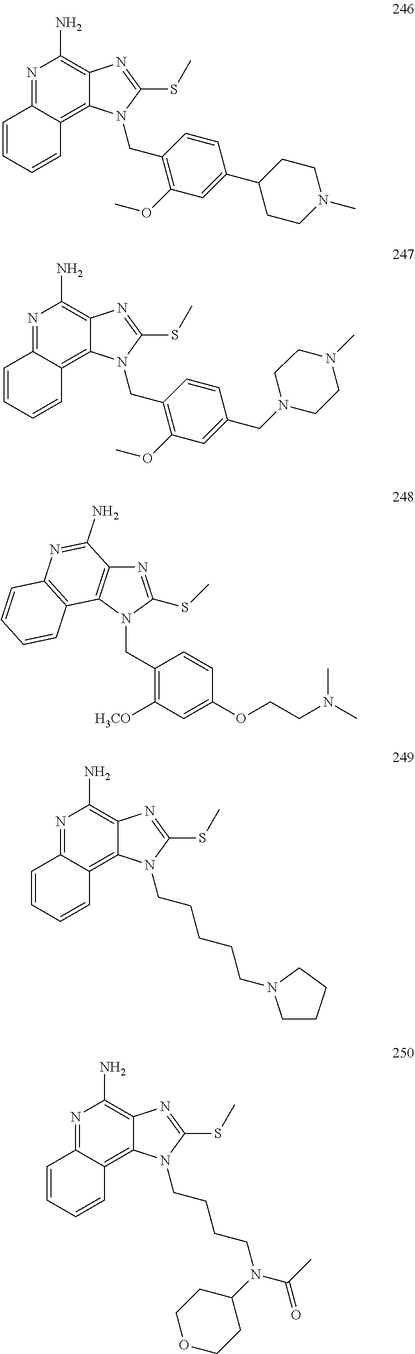 [SMILES: COc1cc(C2CCN(C)CC2)ccc1Cn1c(SC)nc2c(N)nc3ccccc3c21 ; COc1cc(CN2CCN(C)CC2)ccc1Cn1c(SC)nc2c(N)nc3ccccc3c21 ; COc1cc(OCCN(C)C)ccc1Cn1c(SC)nc2c(N)nc3ccccc3c21 ; CSc1nc2c(N)nc3ccccc3c2n1CCCCCN1CCCC1 ; CSc1nc2c(N)nc3ccccc3c2n1CCCCN(C(C)=O)C1CCOCC1]

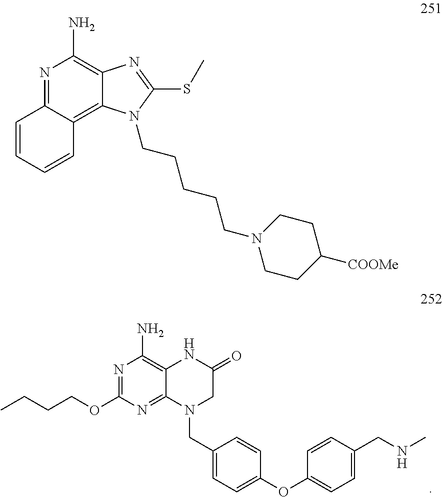 [SMILES: COC(=O)C1CCN(CCCCCn2c(SC)nc3c(N)nc4ccccc4c32)CC1 ; CCCCOc1nc(N)c2c(n1)N(Cc1ccc(Oc3ccc(CNC)cc3)cc1)CC(=O)N2]

53. A pharmaceutical composition comprising the compound, and the stereoisomer, the optical isomer, the pharmaceutically acceptable salt, the prodrug, and the solvate thereof according to claim 1.

54. Use of the compound, and the stereoisomer, the optical isomer, the pharmaceutically acceptable salt, the prodrug, and the solvate thereof according to claim 1, for manufacturing a medicament for the prevention and/or treatment of diseases at least partially mediated by a TLR7 agonist, preferably for manufacturing a medicament for the prevention and/or treatment of diseases mediated by a TLR7 agonist, more preferably for manufacturing a medicament for the treatment of diseases mediated by a TLR7 agonist, and further preferably for manufacturing a medicament for the treatment of cancers or virus-infected diseases.

55. The use according to claim 54, wherein, the virus is selected from Dengue virus, yellow fever virus, West Nile virus, Japanese encephalitis virus, tick-borne encephalitis virus, Kunjin virus, Murray Valley encephalitis virus, Saint Louis encephalitis virus, omsk haemorrhagic fever virus, bovine viral diarrhea virus, Zika virus, HIV, HBV, HCV, HPV, RSV, SARS, and influenza virus.
